(19) United States
(12) Patent Application Publication
(10) Pub. No.: US 2012/0329784 A1
(43) Pub. Date: Dec. 27, 2012

(54) COMPOUNDS AND METHODS

(76) Inventors: Lara S. Kallander, King of Prussia, PA (US); Brian Griffin Lawhorn, King of Prussia, PA (US); Joanne Philp, King of Prussia, PA (US); Yongdong Zhao, King of Prussia, PA (US)

(21) Appl. No.: 13/520,861

(22) PCT Filed: Jan. 11, 2011
(86) PCT No.: PCT/US11/20798

§ 371 Date: Jul. 6, 2012

Related U.S. Application Data
(60) Provisional application No. 61/294,637, filed on Jan. 13, 2010.

(51) Int. Cl. A61K 31/506; C07D 413/12; A61K 31/5377; A61P 9/04; A61K 31/551; C07D 417/12; A61K 31/541; C07D 239/48; C07D 403/12

(52) U.S. Cl. 514/218; 544/327; 514/256; 544/122; 514/235.8; 544/295; 514/252.14; 544/296; 540/575; 544/58.4; 514/227.8

(57) ABSTRACT

Disclosed are compounds having the formula:

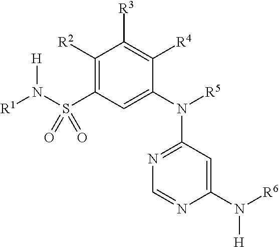

wherein R¹, R², R³, R⁴, R⁵, and R⁶ are as defined herein, and methods of making and using the same.

### FIELD OF THE INVENTION

The present invention relates to compounds that inhibit TNNI3K and methods of making and using the same. Specifically, the present invention relates to 4,6-diaminopyrimidines as TNNI3K inhibitors.

### BACKGROUND OF THE INVENTION

Cardiac troponin I-interacting kinase (TNNI3K), also known as CARK (for cardiac ankyrin repeat kinase), is a protein kinase that exhibits highly selective expression for cardiac tissues and has been shown to interact with components of the sarcomere, including troponin I (Zhao, Y. et al., J. Mol. Med., 2003, 81, 297-304; Feng, Y. et al., Gen. Physiol. Biophys., 2007, 26, 104-109; Wang, H. et al., J. Cell. Mol. Med., 2008, 12, 304-315). Although substrates for TNNI3K have not been identified to date, recent reports suggest that this protein does play a role in the development of pressure-induced cardiomyocyte hypertrophy and contractile dysfunction (Wheeler, F. C. et al., Mamm. Genome, 2005, 16, 414-423; Wang, X. et al. “TNNI3K, a cardiac-specific kinase, promotes cardiac hypertrophy in vivo”, Poster presentation at the 2006 Scientific Sessions of the American Heart Association, Chicago, Ill., Wheeler, F. C. et al., PLos Genet, 2009, 5(9), e1000647; and Pu, W. T., PLos Genet, 2009, 5(9), e1000643). Inhibition of the kinase activity of TNNI3K may disrupt these signaling pathways, and enable the mitigation and/or reversal of cardiac hypertrophy seen in patients with progressively worsening heart failure.

In response to mechanical, neurohormonal, and genetic stimuli, the heart will undergo hypertrophy, or muscle growth and remodeling, in order to maintain sufficient cardiac output to meet tissue oxygen demands. While these structural changes are initially seen as compensatory, sustained dysregulation of hypertrophic signaling can lead to heart failure, the pathophysiological state in which the heart can no longer adequately function as a pump (Mudd, J. O. and Kass, D. A., Nature, 2008, 451, 919-928). Prevention or reversal of pathological cardiac hypertrophy has the potential to delay or prevent the development of congestive heart failure (McKinsey, T. A. and Kass, D. A., Nat. Rev. Drug Discov., 2007, 6, 617-635; Kaye, D. M. and Krum, H., Nat. Rev. Drug Discov., 2007, 6, 127-139).

Heart failure is responsible for a reduced quality of life and premature death in a significant proportion of sufferers, and is characterized by impaired cardiac function either due to reduced pump function (systolic dysfunction) or reduced filling (diastolic dysfunction). Congestive heart failure (CHF) is characterized by impaired left ventricular function, increased peripheral and pulmonary vascular resistance and reduced exercise tolerance and dyspnea. The prevalence of heart failure is anticipated to increase with ageing populations, prompting a need for new and improved methods of treating heart failure.

### SUMMARY OF THE INVENTION

The invention is directed to novel diaminopyrimidines. Specifically, the invention is directed to compounds according to Formula I:

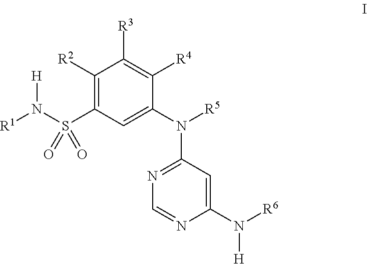

wherein:

R¹ is (C₁-C₄)alkyl;

R² is hydrogen or halogen;

R³ is hydrogen, halogen, (C₁-C₄)alkyl, (C₁-C₄)haloalkyl, (C₃-C₆)cycloalkyl, aryl, hydroxyl, hydroxy(C₁-C₄)alkyl-, (C₁-C₄)alkoxy, (C₁-C₄)alkoxy(C₁-C₄)alkyl-, (C₁-C₄)haloalkoxy, (C₃-C₆)cycloalkyloxy, (C₁-C₄)alkylthio-, amino, (C₁-C₄)alkylamino, or ((C₁-C₄)alkyl)((C₁-C₄)alkyl)amino;

R⁴ is hydrogen, halogen, (C₁-C₈)alkyl, (C₁-C₈)haloalkyl, (C₃-C₈)cycloalkyl, hydroxyl, hydroxy(C₁-C₈)alkyl-, (C₁-C₈)alkoxy, (C₁-C₄)alkoxy(C₁-C₈)alkyl-, (C₁-C₈)haloalkoxy, (C₃-C₈)cycloalkyloxy, (C₁-C₈)alkylthio-, (C₁-C₈)haloalkylthio-, —SO₂(C₁-C₄)alkyl, amino, —NHR⁷, or —NR⁷R⁸;

R⁵ is hydrogen;

or R⁴ and R⁵ taken together with atoms through which they are connected form a 5 or 6 membered ring, optionally containing one or two additional heteroatoms selected from N, O and S, which ring may be unsubstituted or substituted with one to three substituents independently selected from (C₁-C₄)alkyl, (C₁-C₄)haloalkyl, hydroxy(C₁-C₄)alkyl-, oxo, hydroxyl, (C₁-C₄)alkoxy, (C₁-C₄)haloalkoxy, and (C₁-C₄)alkylthio-;

R⁶ is (C₁-C₈)alkyl, (C₂-C₈)alkenyl, (C₂-C₈)alkynyl, (C₃-C₈)cycloalkyl, aryl, or heteroaryl, wherein any aryl or heteroaryl group is optionally substituted one to three times, independently, by halogen, (C₁-C₆)alkyl, (C₂-C₆)alkenyl, (C₂-C₆)alkynyl, (C₃-C₆)cycloalkyl, (C₁-C₄)haloalkyl, cyano, —CO(C₁-C₄)alkyl, —CO₂H, —CO₂R⁷, —CONH₂, —CONHR⁷, —CONR⁷R⁸, HO₂C(C₁-C₂)alkyl-, R⁷O₂C(C₁-C₂)alkyl-, —SR⁷, —SO₂(C₁-C₄)alkyl, —SO₂NH₂, —SO₂NHR⁷, —SO₂NR⁷R⁸, nitro, amino, —NHR⁷, —NR⁷R⁸, amino(C₁-C₂)alkyl-, R⁷HN(C₁-C₂)alkyl-, R⁷R⁸N(C₁-C₂)alkyl-, —NHCO(C₁-C₄)alkyl, —NHSO₂(C₁-C₄)alkyl, oxo, hydroxyl, —OR⁷, hydroxy(C₁-C₂)alkyl-, R⁷O(C₁-C₂)alkyl-, cyano(C₁-C₂)alkyl-, aryl, heteroaryl, or heteroaryl(C₁-C₂)alkyl-, wherein any said aryl or heteroaryl is optionally substituted one to three times, independently, by halogen, (C₁-C₆)alkyl, (C₃-C₆)cycloalkyl, (C₁-C₄)haloalkyl, cyano, —CO(C₁-C₄)alkyl, —CO₂H, —CO₂R⁷, —CONH₂, —CONHR⁷, —CONR⁷R⁸, —SR⁷, —SO₂(C₁-C₄)alkyl, —SO₂NH₂, —SO₂NHR⁷, —SO₂NR⁷R⁸, nitro, amino, —NHR⁷, —NR⁷R⁸, —NHCO(C₁-C₄)alkyl, —NHSO₂(C₁-C₄)alkyl, oxo, hydroxyl, —OR⁷, hydroxy(C₁-C₂)alkyl-, or R⁷O(C₁-C₂)alkyl-;

R⁷ is (C₁-C₄)alkyl, aryl, heterocycloalkyl, or heterocycloalkyl(C₁-C₂)alkyl, wherein said (C₁-C₄)alkyl is optionally substituted one to three times, independently, by halogen, hydroxyl, (C₁-C₄)alkoxy, amino, (C₁-C₄)alkylamino, ((C₁-C₄)alkyl)((C₁-C₄)alkyl)amino, —CO₂H, —CO₂(C₁-C₄)alkyl, —CONH₂, —CONH(C₁-C₄)alkyl, or —CON((C₁-C₄)alkyl)(C₁-C₄)alkyl); and wherein any heterocycloalkyl is optionally substituted by (C₁-C₄)alkyl; and

R⁸ is (C₁-C₄)alkyl;

or R⁷ and R⁸ taken together with the nitrogen to which they are attached represent a 5-7 membered heterocyclic ring, optionally containing an additional heteroatom selected from oxygen, nitrogen, and sulfur, wherein said ring is optionally substituted one or two times, independently, by halogen, (C₁-C₄)alkyl, (C₁-C₄)haloalkyl, amino, (C₁-C₄)alkylamino, ((C₁-C₄)alkyl)((C₁-C₄)alkyl)amino, hydroxyl, oxo, (C₁-C₄)alkoxy, or (C₁-C₄)alkoxy(C₁-C₄)alkyl;

or a salt thereof.

The compounds of the invention are inhibitors of TNNI3K and can be useful for the treatment of cardiac diseases and disorders, particularly heart failure. Accordingly, the invention is further directed to pharmaceutical compositions comprising a compound of the invention. The invention is still further directed to methods of inhibiting TNNI3K and treatment of conditions associated therewith using a compound of the invention or a pharmaceutical composition comprising a compound of the invention.

### DETAILED DESCRIPTION OF THE INVENTION

As used herein, the term “alkyl” represents a saturated, straight or branched hydrocarbon moiety, which may be unsubstituted or substituted by one or more of the substituents defined herein. Exemplary alkyls include, but are not limited to methyl, ethyl, n-propyl, isopropyl, n-butyl, isobutyl, s-butyl, t-butyl, pentyl, and hexyl. The term “C₁-C₄” refers to an alkyl containing from 1 to 4 carbon atoms.

When the term “alkyl” is used in combination with other substituent groups, such as “haloalkyl”, “hydroxyalkyl”, or “alkoxyalkyl”, the term “alkyl” is intended to encompass a divalent straight or branched-chain hydrocarbon radical.

As used herein, the term “alkenyl” refers to straight or branched hydrocarbon chains containing the specified number of carbon atoms and at least 1 and up to 3 carbon-carbon double bonds. Examples include ethenyl and propenyl.

As used herein, the term “alkynyl” refers to straight or branched hydrocarbon chains containing the specified number of carbon atoms and at least 1 and up to 3 carbon-carbon triple bonds. Examples include ethynyl and propynyl.

As used herein, the term “cycloalkyl” refers to a non-aromatic, saturated, cyclic hydrocarbon ring. The term “(C₃-C₈)cycloalkyl” refers to a non-aromatic cyclic hydrocarbon ring having from three to eight ring carbon atoms. Exemplary “(C₃-C₈)cycloalkyl” groups useful in the present invention include cyclopropyl, cyclobutyl, cyclopentyl, cyclohexyl, cycloheptyl, and cyclooctyl.

“Alkoxy” refers to a group containing an alkyl radical attached through an oxygen linking atom. The term “(C₁-C₄)alkoxy” refers to a straight- or branched-chain hydrocarbon radical having at least 1 and up to 4 carbon atoms attached through an oxygen linking atom. Exemplary “(C₁-C₄)alkoxy” groups useful in the present invention include, but are not limited to, methoxy, ethoxy, n-propoxy, isopropoxy, n-butoxy, s-butoxy, and t-butoxy.

“Alkylthio-” refers to a group containing an alkyl radical attached through a sulfur linking atom. The term “(C₁-C₄)alkylthio-” refers to a straight- or branched-chain hydrocarbon radical having at least 1 and up to 4 carbon atoms attached through a sulfur linking atom. Exemplary “(C₁-C₄)alkylthio-” groups useful in the present invention include, but are not limited to, methylthio-, ethylthio-, n-propylthio-, isopropylthio-, n-butylthio-, s-butylthio-, and t-butylthio-.

“Cycloalkyloxy” refers to a group containing a saturated carbocyclic ring attached through an oxygen linking atom. Examples of “cycloalkyloxy” moieties include, but are not limited to, cyclopropyloxy, cyclobutyloxy, cyclopentyloxy, cyclohexyloxy, and the like.

“Aryl” represents a group or moiety comprising an aromatic, monovalent monocyclic or bicyclic hydrocarbon radical containing from 6 to 10 carbon ring atoms, which may be unsubstituted or substituted by one or more of the substituents defined herein, and to which may be fused to one or more cycloalkyl rings, which may be unsubstituted or substituted by one or more substituents defined herein.

Generally, in the compounds of this invention, aryl is phenyl.

Heterocyclic groups may be heteroaryl or heterocycloalkyl groups.

“Heterocycloalkyl” represents a group or moiety comprising a non-aromatic, monovalent monocyclic or bicyclic radical, which is saturated or partially unsaturated, containing 3 to 10 ring atoms, which includes 1 to 3 heteroatoms selected from nitrogen, oxygen and sulfur, and which may be unsubstituted or substituted by one or more of the substituents defined herein. Illustrative examples of heterocycloalkyls include, but are not limited to, azetidinyl, pyrrolidinyl, pyrazolidinyl, pyrazolinyl, imidazolidinyl, imidazolinyl, oxazolinyl, thiazolinyl, tetrahydrofuranyl, dihydrofuranyl, 1,3-dioxolanyl, piperidinyl, piperazinyl, morpholinyl, thiomorpholinyl, tetrahydropyranyl, dihydropyranyl, 1,3-dioxanyl, 1,4-dioxanyl, 1,3-oxathiolanyl, 1,3-oxathianyl, 1,3-dithianyl, hexahydro-1H-1,4-diazepinyl, azabicylo[3.2.1]octyl, azabicylo[3.3.1]nonyl, azabicylo[4.3.0]nonyl, oxabicylo[2.2.1]heptyl and 1,5,9-triazacyclododecyl.

Generally, in the compounds of this invention, heterocycloalkyl groups are 5-7 membered heterocycloalkyl groups, such as pyrrolidinyl, pyrazolidinyl, pyrazolinyl, imidazolidinyl, imidazolinyl, oxazolinyl, thiazolinyl, tetrahydrofuranyl, dihydrofuranyl, 1,3-dioxolanyl, piperidinyl, piperazinyl, morpholinyl, thiomorpholinyl, tetrahydropyranyl, dihydropyranyl, and hexahydro-1H-1,4-diazepinyl.

“Heteroaryl” represents a group or moiety comprising an aromatic monovalent monocyclic or bicyclic radical, containing 5 to 10 ring atoms, including 1 to 4 heteroatoms selected from nitrogen, oxygen and sulfur, which may be unsubstituted or substituted by one or more of the substituents defined herein. This term also encompasses bicyclic heterocyclic-aryl compounds containing an aryl ring moiety fused to a heterocycloalkyl ring moiety, containing 5 to 10 ring atoms, including 1 to 4 heteroatoms selected from nitrogen, oxygen and sulfur, which may be unsubstituted or substituted by one or more of the substituents defined herein. Illustrative examples of heteroaryls include, but are not limited to, furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, triazinyl, benzofuranyl, isobenzofuryl, 2,3-dihydrobenzofuryl, 1,3-benzodioxolyl, dihydrobenzodioxinyl, benzothienyl, indolizinyl, indolyl, isoindolyl, dihydroindolyl, dihydroisoindolyl, chromenyl, benzimidazolyl, dihydrobenzimidazolyl, benzoxazolyl, dihydrobenzoxazolyl, benzothiazolyl, dihydrobenzothiazolyl, benzoisothiazolyl, dihydrobenzoisothiazolyl, indazolyl, imidazopyridinyl, pyrazolopyridinyl, benzotriazolyl, triazolopyridinyl, purinyl, quinolinyl, dihydroquinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, and pteridinyl.

Generally, the heteroaryl groups present in the compounds of this invention are 5-membered and/or 6-membered monocyclic heteroaryl groups. Selected 5-membered heteroaryl groups contain one nitrogen, oxygen or sulfur ring heteroatom, and optionally contain 1, 2, or 3 additional nitrogen ring atoms. Selected 6-membered heteroaryl groups contain 1, 2, or 3 nitrogen ring heteroatoms. Selected 5- or 6-membered heteroaryl groups include furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, and triazinyl.

“Oxo” represents a double-bonded oxygen moiety; for example, if attached directly to a carbon atom forms a carbonyl moiety (C═O).

The terms “halogen” and “halo” represent chloro, fluoro, bromo, or iodo substituents. “Hydroxy” or “hydroxyl” is intended to mean the radical —OH.

As used herein, the term “compound(s) of the invention” means a compound of Formula I (as defined above) in any form, i.e., any salt or non-salt form (e.g., as a free acid or base form, or as a pharmaceutically acceptable salt thereof) and any physical form thereof (e.g., including non-solid forms (e.g., liquid or semi-solid forms), and solid forms (e.g., amorphous or crystalline forms, specific polymorphic forms, solvates, including hydrates (e.g., mono-, di- and hemi-hydrates)), and mixtures of various forms.

As used herein, the term “optionally substituted” means that the groups may be either unsubstituted or substituted with one or more of the specified substituents.

The alternative definitions for the various groups and substituent groups of Formula I provided throughout the specification are intended to particularly describe each compound species disclosed herein, individually, as well as groups of one or more compound species. The scope of this invention includes any combination of these group and substituent group definitions.

Suitably, R¹ is (C₁-C₄)alkyl. In a specific embodiment of this invention, R¹ is methyl.

Suitably, R² is hydrogen or halogen. In a specific embodiment of this invention, R² is hydrogen or fluorine. In a further specific embodiment of this invention, R² is hydrogen.

Suitably, R³ is hydrogen, halogen, (C₁-C₄)alkyl, (C₁-C₄)haloalkyl, (C₃-C₆)cycloalkyl, aryl, hydroxyl, hydroxy(C₁-C₄)alkyl-, (C₁-C₄)alkoxy, (C₁-C₄)alkoxy(C₁-C₄)alkyl-, (C₁-C₄)haloalkoxy, (C₃-C₆)cycloalkyloxy, (C₁-C₄)alkylthio-, amino, (C₁-C₄)alkylamino, or ((C₁-C₄)alkyl)((C₁-C₄)alkyl)amino. In another embodiment of this invention, R³ is hydrogen, halogen, (C₁-C₄)alkyl, (C₁-C₄)haloalkyl, phenyl, (C₁-C₄)alkoxy, (C₁-C₄)alkylthio-, or ((C₁-C₄)alkyl)((C₁-C₄)alkyl)amino. In a specific embodiment of this invention, R³ is hydrogen, chlorine, or dimethylamino. In a further specific embodiment of this invention, R³ is hydrogen. In yet a further specific embodiment of this invention, R² and R³ are each hydrogen.

Suitably, R⁴ is hydrogen, halogen, (C₁-C₈)alkyl, (C₁-C₈)haloalkyl, (C₃-C₈)cycloalkyl, hydroxyl, hydroxy(C₁-C₈)alkyl-, (C₁-C₈)alkoxy, (C₁-C₄)alkoxy(C₁-C₈)alkyl-, (C₁-C₈)haloalkoxy, (C₃-C₈)cycloalkyloxy, (C₁-C₈)alkylthio-, (C₁-C₈)haloalkylthio-, —SO₂(C₁-C₄)alkyl, amino, —NHR⁷, or —NR⁷R⁸. In another embodiment of this invention, R⁴ is hydrogen, halogen, (C₁-C₈)alkyl, (C₁-C₈)haloalkyl, (C₃-C₈)cycloalkyl, hydroxyl, hydroxy(C₁-C₈)alkyl-, (C₁-C₈)alkoxy, (C₁-C₄)alkoxy(C₁-C₈)alkyl-, (C₁-C₈)haloalkoxy, (C₃-C₈)cycloalkyloxy, (C₁-C₈)alkylthio-, (C₁-C₈)haloalkylthio-, —SO₂(C₁-C₄)alkyl, amino, (C₁-C₄)alkylamino, (C₁-C₄)haloalkylamino, ((C₁-C₄)alkyl)((C₁-C₄)alkyl)amino, ((C₁-C₄)alkyl)((C₁-C₄)haloalkyl)amino, ((C₁-C₄)haloalkyl)((C₁-C₄)haloalkyl)amino, pyrrolidinyl, imidazolidinyl, pyrazolidinyl, piperidinyl, piperazinyl, morpholinyl, or thiomorpholinyl, wherein said pyrrolidinyl, imidazolidinyl, pyrazolidinyl, piperidinyl, piperazinyl, morpholinyl, or thiomorpholinyl is optionally substituted one or two times, independently, by halogen, (C₁-C₄)alkyl, (C₁-C₄)haloalkyl, amino, (C₁-C₄)alkylamino, ((C₁-C₄)alkyl)((C₁-C₄)alkyl)amino, hydroxyl, oxo, (C₁-C₄)alkoxy, or (C₁-C₄)alkoxy(C₁-C₄)alkyl. In a further embodiment of this invention, R⁴ is hydrogen, halogen, (C₁-C₈)alkyl, (C₁-C₈)haloalkyl, (C₃-C₈)cycloalkyl, hydroxyl, hydroxy(C₁-C₈)alkyl-, (C₁-C₈)alkoxy, (C₁-C₄)alkoxy(C₁-C₈)alkyl-, (C₁-C₈)haloalkoxy, (C₃-C₈)cycloalkyloxy, (C₁-C₈)alkylthio-, —SO₂(C₁-C₄)alkyl, amino, (C₁-C₄)alkylamino, ((C₁-C₄)alkyl)((C₁-C₄)alkyl)amino, pyrrolidinyl, imidazolidinyl, pyrazolidinyl, piperidinyl, piperazinyl, morpholinyl, or thiomorpholinyl. In specific embodiments of this invention, R⁴ is hydrogen, fluorine, chlorine, hydroxyl, methoxy, ethoxy, n-propyloxy, isopropyloxy, isobutyloxy, 3-methyl-2-butyloxy, 3-pentyloxy, trifluoromethoxy, 2,2,2-trifluoroethoxy, 1,1,1-trifluoro-2-propyloxy, 3,3,3-trifluoro-1-propyloxy, 1,1,1-trifluoro-2-methyl-2-propyloxy, 1,1,1,3,3,3-hexafluoro-2-methyl-2-propyloxy, cyclopentyloxy, cyclohexyloxy, methylthio-, ethylthio-, isobutylthio-, 2,2,2-trifluoroethylthio-, methylsulfone, ethylsulfone, isopropylsulfone, isobutylsulfone, tert-butylsulfone, amino, dimethylamino, ethylmethylamino, diethylamino, methyl-2,2,2-trifluoroethylamino, 2-methylpyrrolidin-1-yl, (R)-2-trifluoromethylpyrrolidin-1-yl, 2,5-dimethylpyrrolidin-1-yl, 3,3-difluoropyrrolidin-1-yl, 3,3-difluoropiperidin-1-yl, or morpholin-4-yl.

In a further embodiment of the invention, R⁴ and R⁵ taken together with atoms through which they are connected form a 5 or 6 membered ring, optionally containing one or two additional heteroatoms selected from N, O and S, which ring may be unsubstituted or substituted with one to three substituents independently selected from (C₁-C₄)alkyl, (C₁-C₄)haloalkyl, hydroxy(C₁-C₄)alkyl-, oxo, hydroxyl, (C₁-C₄)alkoxy, (C₁-C₄)haloalkoxy, and (C₁-C₄)alkylthio-. In yet a further embodiment of the invention, R⁴ and R⁵ taken together with atoms through which they are connected form a partially saturated 5 or 6 membered ring, optionally containing one or two additional heteroatoms selected from N, O and S, which ring may be unsubstituted or substituted with one to three substituents independently selected from (C₁-C₄)alkyl, (C₁-C₄)haloalkyl, hydroxy(C₁-C₄)alkyl-, (C₁-C₄)alkoxy, (C₁-C₄)haloalkoxy, and (C₁-C₄)alkylthio-. In a specific embodiment of this invention, R⁴ and R⁵ taken together represent —CH₂CH₂—, —C(CH₃)₂CH₂—, —CH═CH—, —NH(C═O)—, or —N═CH—. In a further specific embodiment of this invention, R⁴ and R⁵ taken together represent —CH₂CH₂—.

Suitably, R⁶ is (C₁-C₈)alkyl, (C₂-C₈)alkenyl, (C₂-C₈)alkynyl, (C₃-C₈)cycloalkyl, aryl, or heteroaryl, wherein any aryl or heteroaryl group is optionally substituted one to three times, independently, by halogen, (C₁-C₆)alkyl, (C₂-C₆)alkenyl, (C₂-C₆)alkynyl, (C₃-C₆)cycloalkyl, (C₁-C₄)haloalkyl, cyano, —CO(C₁-C₄)alkyl, —CO₂H, —CO₂R⁷, —CONH₂, —CONHR⁷, —CONR⁷R⁸, HO₂C(C₁-C₂)alkyl-, R⁷O₂C(C₁-C₂)alkyl-, —SO₂(C₁-C₄)alkyl, —SO₂NH₂, —SO₂NHR⁷, —SO₂NR⁷R⁸, nitro, amino, —NHR⁷, —NR⁷R⁸, amino(C₁-C₂)alkyl-, R⁷HN(C₁-C₂)alkyl-, R⁷R⁸N(C₁-C₂)alkyl-, —NHCO(C₁-C₄)alkyl, —NHSO₂(C₁-C₄)alkyl, oxo, hydroxyl, —OR⁷, hydroxy(C₁-C₂)alkyl-, R⁷O(C₁-C₂)alkyl-, cyano(C₁-C₂)alkyl-, aryl, heteroaryl, or heteroaryl(C₁-C₂)alkyl-, wherein any said aryl or heteroaryl is optionally substituted one to three times, independently, by halogen, (C₁-C₆)alkyl, (C₃-C₆)cycloalkyl, (C₁-C₄)haloalkyl, cyano, —CO(C₁-C₄)alkyl, —CO₂H, —CO₂R⁷, —CONH₂, —CONHR⁷, —CONR⁷R⁸, —SR⁷, —SO₂(C₁-C₄)alkyl, —SO₂NH₂, —SO₂NHR⁷, —SO₂NR⁷R⁸, nitro, amino, —NHR⁷, —NR⁷R⁸, —NHCO(C₁-C₄)alkyl, —NHSO₂(C₁-C₄)alkyl, oxo, hydroxyl, —OR⁷, hydroxy(C₁-C₂)alkyl-, or R⁷O(C₁-C₂)alkyl-.

In another embodiment of this invention, R⁶ is (C₁-C₆)alkyl, phenyl, dihydroindenyl, tetrahydronaphthalenyl, oxazolyl, thiazolyl, thiadiazolyl, pyridinyl, pyrimidinyl, indolyl, indazolyl, dihydroindolyl, dihydroisoindolyl, chromenyl, dihydrobenzimidazolyl, dihydrobenzoxazolyl, benzothiazolyl, dihydrobenzoisothiazolyl, quinolinyl, isoquinolinyl, dihydroquinolinyl, tetrahydroquinolinyl, tetrahydroisoquinolinyl, benzodioxolyl, or dihydrobenzodioxinyl, wherein said phenyl, dihydroindenyl, tetrahydronaphthalenyl, oxazolyl, thiazolyl, thiadiazolyl, pyridinyl, pyrimidinyl, indolyl, indazolyl, dihydroindolyl, dihydroisoindolyl, chromenyl, dihydrobenzimidazolyl, dihydrobenzoxazolyl, benzothiazolyl, dihydrobenzoisothiazolyl, quinolinyl, isoquinolinyl, dihydroquinolinyl, tetrahydroquinolinyl, tetrahydroisoquinolinyl, benzodioxolyl, or dihydrobenzodioxinyl group is optionally substituted one to three times, independently, by halogen, (C₁-C₆)alkyl, (C₂-C₆)alkenyl, (C₂-C₆)alkynyl, (C₃-C₆)cycloalkyl, (C₁-C₄)haloalkyl, cyano, —CO(C₁-C₄)alkyl, —CO₂H, —CO₂R⁷, —CONH₂, —CONHR⁷, —CONR⁷R⁸, HO₂C(C₁-C₂)alkyl-, R⁷O₂C(C₁-C₂)alkyl-, cyano(C₁-C₂)alkyl-, —SO₂(C₁-C₄)alkyl, —SO₂NH₂, —SO₂NHR⁷, —SO₂NR⁷R⁸, nitro, amino, —NHR⁷, —NR⁷R⁸, amino(C₁-C₂)alkyl-, R⁷R⁸N(C₁-C₂)alkyl-, R⁷R⁸N(C₁-C₂)alkyl-, triazolyl(C₁-C₂)alkyl-, —NHCO(C₁-C₄)alkyl, —NHSO₂(C₁-C₄)alkyl, oxo, hydroxyl, —OR⁷, hydroxy(C₁-C₂)alkyl-, R⁷O(C₁-C₂)alkyl-, phenyl, thienyl, pyrazolyl, imidazolyl, oxazolyl, thiazolyl, or pyridinyl, wherein said phenyl, thienyl, pyrazolyl, imidazolyl, oxazolyl, thiazolyl, or pyridinyl is optionally substituted one or two times, independently, by halogen, (C₁-C₆)alkyl, (C₃-C₆)cycloalkyl, (C₁-C₄)haloalkyl, cyano, —CO(C₁-C₄)alkyl, —CO₂H, —CO₂R⁷, —CONH₂, —CONHR⁷, —CONR⁷R⁸, —SO₂(C₁-C₄)alkyl, —SO₂NH₂, —SO₂NHR⁷, —SO₂NR⁷R⁸, nitro, amino, —NHR⁷, —NR⁷R⁸, —NHCO(C₁-C₄)alkyl, —NHSO₂(C₁-C₄)alkyl, oxo, hydroxyl, hydroxy(C₁-C₂)alkyl-, or R⁷O(C₁-C₂)alkyl-.

In yet another embodiment of this invention, R⁶ is (C₁-C₆)alkyl, phenyl, oxazolyl, thiazolyl, thiadiazolyl, pyridinyl, indolyl, indazolyl, dihydroindolyl, dihydrobenzimidazolyl, dihydrobenzoxazolyl, benzothiazolyl, dihydrobenzoisothiazolyl, quinolinyl, isoquinolinyl, tetrahydroquinolinyl, tetrahydroisoquinolinyl, or dihydrobenzodioxinyl, wherein said phenyl, oxazolyl, thiazolyl, thiadiazolyl, pyridinyl, indolyl, indazolyl, dihydroindolyl, dihydrobenzimidazolyl, dihydrobenzoxazolyl, benzothiazolyl, dihydrobenzoisothiazolyl, quinolinyl, isoquinolinyl, tetrahydroquinolinyl, tetrahydroisoquinolinyl, or dihydrobenzodioxinyl group is optionally substituted one or two times, independently, by halogen, (C₁-C₆)alkyl, (C₃-C₆)cycloalkyl, (C₁-C₄)haloalkyl, cyano, —CO(C₁-C₄)alkyl, —CO₂H, —CO₂R⁷, —CONH₂, —CONHR⁷, —CONR⁷R⁸, HO₂C(C₁-C₂)alkyl-, R⁷O₂C(C₁-C₂)alkyl-, —SO₂(C₁-C₄)alkyl, —SO₂NH₂, —SO₂NHR⁷, —SO₂NR⁷R⁸, nitro, amino, —NHR⁷, —NR⁷R⁸, amino(C₁-C₂)alkyl-, R⁷R⁸N(C₁-C₂)alkyl-, R⁷R⁸N(C₁-C₂)alkyl-, —NHCO(C₁-C₄)alkyl, —NHSO₂(C₁-C₄)alkyl, oxo, hydroxyl, —OR⁷, hydroxy(C₁-C₂)alkyl-, R⁷O(C₁-C₂)alkyl-, phenyl, thienyl, pyrazolyl, imidazolyl, or pyridinyl, wherein said phenyl, thienyl, pyrazolyl, imidazolyl, or pyridinyl is optionally substituted one or two times, independently, by halogen, (C₁-C₆)alkyl, (C₃-C₆)cycloalkyl, (C₁-C₄)haloalkyl, cyano, —CO(C₁-C₄)alkyl, —CO₂H, —CO₂R⁷, —CONH₂, —CONHR⁷, —CONR⁷R⁸, —SO₂(C₁-C₄)alkyl, —SO₂NH₂, —SO₂NHR⁷, —SO₂NR⁷R⁸, nitro, amino, —NHR⁷, —NR⁷R⁸, —NHCO(C₁-C₄)alkyl, —NHSO₂(C₁-C₄)alkyl, oxo, hydroxyl, —OR⁷, hydroxy(C₁-C₂)alkyl-, or R⁷O(C₁-C₂)alkyl-.

In a further embodiment of this invention, R⁶ is phenyl optionally substituted one to three times, independently, by halogen, (C₁-C₆)alkyl, (C₂-C₆)alkenyl, (C₂-C₆)alkynyl, (C₃-C₆)cycloalkyl, (C₁-C₄)haloalkyl, cyano, —CO(C₁-C₄)alkyl, —CO₂H, —CO₂R⁷, —CONH₂, —CONHR⁷, —CONR⁷R⁶, HO₂C(C₁-C₂)alkyl-, R⁷O₂C(C₁-C₂)alkyl-, cyano(C₁-C₂)alkyl-, —SO₂(C₁-C₄)alkyl, —SO₂NH₂, —SO₂NHR⁷, —SO₂NR⁷R⁸, nitro, amino, —NHR⁷, —NR⁷R⁸, amino(C₁-C₂)alkyl-, R⁷R⁸N(C₁-C₂)alkyl-, R⁷R⁸N(C₁-C₂)alkyl-, triazolyl(C₁-C₂)alkyl-, —NHCO(C₁-C₄)alkyl, —NHSO₂(C₁-C₄)alkyl, oxo, hydroxyl, —OR⁷, hydroxy(C₁-C₂)alkyl-, R⁷O(C₁-C₂)alkyl-, phenyl, thienyl, pyrazolyl, imidazolyl, oxazolyl, thiazolyl, or pyridinyl, wherein said phenyl, thienyl, pyrazolyl, imidazolyl, oxazolyl, thiazolyl, or pyridinyl is optionally substituted one or two times, independently, by halogen, (C₁-C₆)alkyl, (C₃-C₆)cycloalkyl, (C₁-C₄)haloalkyl, cyano, —CO(C₁-C₄)alkyl, —CO₂H, —CO₂R⁷, —CONH₂, —CONHR⁷, —CONR⁷R⁸, —SO₂(C₁-C₄)alkyl, —SO₂NH₂, —SO₂NHR⁷, —SO₂NR⁷R⁸, nitro, amino, —NHR⁷, —NR⁷R⁸, —NHCO(C₁-C₄)alkyl, —NHSO₂(C₁-C₄)alkyl, oxo, hydroxyl, —OR⁷, hydroxy(C₁-C₂)alkyl-, or R⁷O(C₁-C₂)alkyl-.

In yet a further embodiment of this invention, R⁶ is phenyl optionally substituted one or two times, independently, by halogen, (C₁-C₆)alkyl, (C₃-C₆)cycloalkyl, (C₁-C₄)haloalkyl, cyano, —CO(C₁-C₄)alkyl, —CO₂H, —CO₂R⁷, —CONH₂, —CONHR⁷, —CONR⁷R⁸, HO₂C(C₁-C₂)alkyl-, R⁷O₂C(C₁-C₂)alkyl-, —SO₂(C₁-C₄)alkyl, —SO₂NH₂, —SO₂NHR⁷, —SO₂NR⁷R⁸, nitro, amino, —NHR⁷, —NR⁷R⁸, amino(C₁-C₂)alkyl-, R⁷R⁸N(C₁-C₂)alkyl-, R⁷R⁸N(C₁-C₂)alkyl-, —NHCO(C₁-C₄)alkyl, —NHSO₂(C₁-C₄)alkyl, oxo, hydroxyl, —OR⁷, hydroxy(C₁-C₂)alkyl-, R⁷O(C₁-C₂)alkyl-, phenyl, thienyl, pyrazolyl, imidazolyl, or pyridinyl, wherein said phenyl, thienyl, pyrazolyl, imidazolyl, or pyridinyl is optionally substituted one or two times, independently, by halogen, (C₁-C₆)alkyl, (C₃-C₆)cycloalkyl, (C₁-C₄)haloalkyl, cyano, —CO(C₁-C₄)alkyl, —CO₂H, —CO₂R⁷, —CONH₂, —CONHR⁷, —CONR⁷R⁸, —SO₂(C₁-C₄)alkyl, —SO₂NH₂, —SO₂NHR⁷, —SO₂NR⁷R⁸, nitro, amino, —NHR⁷, —NR⁷R⁸, —NHCO(C₁-C₄)alkyl, —NHSO₂(C₁-C₄)alkyl, oxo, hydroxyl, —OR⁷, hydroxy(C₁-C₂)alkyl-, or R⁷O(C₁-C₂)alkyl-.

In still a further embodiment of this invention, R⁶ is pyridinyl optionally substituted one or two times, independently, by halogen, (C₁-C₆)alkyl, (C₃-C₆)cycloalkyl, (C₁-C₄)haloalkyl, cyano, —CO(C₁-C₄)alkyl, —CO₂H, —CO₂R⁷, —CONH₂, —CONHR⁷, —CONR⁷R⁸, HO₂C(C₁-C₂)alkyl-, R⁷O₂C(C₁-C₂)alkyl-, —SO₂(C₁-C₄)alkyl, —SO₂NH₂, —SO₂NHR⁷, —SO₂NR⁷R⁸, nitro, amino, —NHR⁷, —NR⁷R⁸, amino(C₁-C₂)alkyl-, R⁷R⁸N(C₁-C₂)alkyl-, R⁷R⁸N(C₁-C₂)alkyl-, —NHCO(C₁-C₄)alkyl, —NHSO₂(C₁-C₄)alkyl, oxo, hydroxyl, —OR⁷, hydroxy(C₁-C₂)alkyl-, or R⁷O(C₁-C₂)alkyl-. In still a further embodiment of this invention, R⁶ is pyridinyl optionally substituted one or two times, independently, by halogen, (C₁-C₄)alkyl, (C₁-C₄)haloalkyl, or cyano.

In a specific embodiment of this invention, R⁶ is methyl, ethyl, oxazol-2-yl, oxazol-5-yl, 4-methyl-oxazol-2-yl, thiazol-2-yl, 4-trifluoromethyl-thiazol-2-yl, 4-isopropyl-thiazol-2-yl, 5-methyl-thiazol-2-yl, 4-carboxymethyl-thiazol-2-yl, 4-(methoxycarbonyl)methyl-thiazol-2-yl, 5-carboxy-thiazol-2-yl, 1,3,4-thiadiazol-2-yl, pyridin-2-yl, 3-fluoro-pyridin-2-yl, 5-fluoro-pyridin-2-yl, 5-chloro-pyridin-2-yl, 5-isopropyl-pyridin-2-yl, 5-trifluoromethyl-pyridin-2-yl, 5-cyano-pyridin-2-yl, 5-chloro-3-fluoro-pyridin-2-yl, 3,5-dichloro-pyridin-2-yl, 4,5-dichloro-pyridin-2-yl, 5-chloro-4-methyl-pyridin-2-yl, 5-chloro-6-methyl-pyridin-2-yl, 5-bromo-6-methyl-pyridin-2-yl, 6-bromo-4-methyl-pyridin-2-yl, pyridin-3-yl, 5-methyl-pyridin-3-yl, 6-trifluoromethyl-pyridin-3-yl, 5-methylsulfonamide-pyridin-3-yl, pyridin-4-yl, pyrimidin-4-yl, 2,3-dihydro-1H-inden-5-yl, 5-oxo-5,6,7,8-tetrahydronaphthalen-2-yl, 1H-indol-5-yl, 1H-indol-6-yl, 1-acetyl-2,3-dihydro-1H-indol-6-yl, 2-methyl-1,3-dioxo-2,3-dihydro-1H-isoindol-5-yl, 1H-indazol-5-yl, 1H-indazol-6-yl, 3-methyl-1H-indazol-6-yl, 2-oxo-2,3-dihydro-1H-indol-5-yl, 2-oxo-2,3-dihydro-1H-indol-6-yl, 2-methyl-4-oxo-4H-chromen-7-yl, 4-methyl-2-oxo-2H-chromen-7-yl, 2-oxo-2,3-dihydro-1H-benzimidazol-5-yl, 2-oxo-2,3-dihydro-1,3-benzoxazol-6-yl, 2-methyl-1,3-benzothiazol-5-yl, 1,3-benzothiazol-5-yl, 1,3-benzothiazol-6-yl, 1,1-dioxido-2,3-dihydro-1,2-benzisothiazol-6-yl, quinolin-2-yl, quinolin-6-yl, isoquinolin-3-yl, 4-methyl-2-oxo-1,2-dihydroquinolin-7-yl, 2-methyl-1,2,3,4-tetrahydroisoquinolin-7-yl, 2-oxo-1,2,3,4-tetrahydroquinolin-7-yl, 1,3-benzodioxol-5-yl, 2,3-dihydro-1,4-benzodioxin-6-yl, phenyl, 2-fluorophenyl, 3-fluorophenyl, 4-fluorophenyl, 3-chlorophenyl, 4-chlorophenyl, 3-bromophenyl, 4-bromophenyl, 3,4-difluorophenyl, 3,4-dichlorophenyl, 3,5-dichlorophenyl, 3-fluoro-4-chlorophenyl, 3-bromo-4-chlorophenyl, 3-bromo-5-chlorophenyl, 3,4,5-trifluorophenyl, 3-methylphenyl, 4-methylphenyl, 3-isopropylphenyl, 4-isopropylphenyl, 4-sec-butylphenyl, 3-tert-butylphenyl, 4-tert-butylphenyl, 3,4-dimethylphenyl, 3,5-dimethylphenyl, 3-fluoro-4-methylphenyl, 4-fluoro-3-methylphenyl, 4-chloro-3-methylphenyl, 3-bromo-5-methylphenyl, 3-ethynylphenyl, 3-trifluoromethylphenyl, 4-trifluoromethylphenyl, 3-fluoro-4-trifluoromethylphenyl, 4-chloro-3-trifluoromethylphenyl, 4-methyl-3-trifluoromethylphenyl, 4-cyclopropylphenyl, 4-(2,2,2-trifluoroethyl)phenyl, 4-(thien-2-yl)phenyl, 4-(1H-pyrazol-1-yl)phenyl, 4-(3,5-dimethyl-1H-pyrazol-1-yl)phenyl, 4-(2-methyl-1H-imidazol-1-yl)phenyl, 4-(oxazol-5-yl)phenyl, 3-(2-methyl-thiazol-4-yl)phenyl, 3-biphenylyl, 3′-aminocarbonyl-3-biphenylyl, 4′-aminocarbonyl-3-biphenylyl, 3′-dimethylamino-3-biphenylyl, 4′-dimethylamino-3-biphenylyl, 4′-morpholin-4-yl-3-biphenylyl, 3′-acetylamino-3-biphenylyl, 4′-acetylamino-3-biphenylyl, 3′-[(methylsulfonyl)amino]-3-biphenylyl, 4′-[(methylsulfonyl)amino]-3-biphenylyl, 3′-[(methylamino)sulfonyl]-3-biphenylyl, 4′-[(methylamino)sulfonyl]-3-biphenylyl, 5-methyl-3-biphenylyl, 4-chloro-3′-morpholin-4-yl-3-biphenylyl, 4-chloro-3′-aminocarbonyl-3-biphenylyl, 3-(4-methoxy-pyridin-3-yl)phenyl, 3-(5-methoxy-pyridin-3-yl)phenyl, 3-(6-methoxy-pyridin-3-yl)phenyl, 3-(6-oxo-pyridin-3-yl)phenyl, 3-(6-dimethylamino-pyridin-3-yl)phenyl, 5-methyl-3-(pyridin-3-yl)phenyl, 4-chloro-3-(pyridin-3-yl)phenyl, 4-(cyanomethyl)phenyl, 3-(1-pyrrolidinylmethyl)phenyl, 3-[(4-methyl-1-piperazinyl)methyl]phenyl, 4-(1H-1,2,4-triazol-1-ylmethyl)phenyl, 4-(4H-1,2,4-triazol-4-ylmethyl)phenyl, 3-acetylphenyl, 4-acetylphenyl, 4-carboxyphenyl, 4-[(methoxy)carbonyl]phenyl, 4-[(isopropoxy)carbonyl]phenyl, 3-aminocarbonylphenyl, 4-aminocarbonylphenyl, 4-(methylamino)carbonylphenyl, 4-(dimethylaminoethylamino)carbonylphenyl, 4-(hydroxyethylamino)carbonylphenyl, 4-(methoxyethylamino)carbonylphenyl, 4-(methoxypropylamino)carbonylphenyl, 4-(carboxymethylamino)carbonylphenyl, 4-[(1-methyl-piperidin-4-yl)amino]carbonylphenyl, 3-(phenylamino)carbonylphenyl, 4-(phenylamino)carbonylphenyl, 4-(dimethylamino)carbonylphenyl, 4-(diethylamino)carbonylphenyl, 4-[N-methyl-N—(N′,N′-dimethylaminoethyl)amino]carbonylphenyl, 4-(pyrrolidin-1-yl)carbonylphenyl, 4-[(3S)-3-(dimethylamino)pyrrolidin-1-yl]carbonylphenyl, 4-[(3R)-3-(dimethylamino)pyrrolidin-1-yl]carbonylphenyl, 4-(4,4-difluoropiperidin-1-yl)carbonylphenyl, 4-(morpholin-4-yl)carbonylphenyl, 4-(thiomorpholin-4-yl)carbonylphenyl, 4-(piperazin-1-yl)carbonylphenyl, 4-(4-methyl-piperazin-1-yl)carbonylphenyl, 4-(4-methoxyethyl-piperazin-1-yl)carbonylphenyl, 4-(4-methyl-hexahydro-1H-1,4-diazepin-1-yl)carbonylphenyl, 4-cyanophenyl, 3-chloro-4-cyanophenyl, 3-nitrophenyl, 3-dimethylaminophenyl, 4-dimethylaminophenyl, 3-(pyrrolidin-1-yl)phenyl, 4-(piperidin-1-yl)phenyl, 4-(piperazin-1-yl)phenyl, 3-(morpholin-4-yl)phenyl, 4-(morpholin-4-yl)phenyl, 3-(4-methyl-piperazin-1-yl)phenyl, 3-(acetylamino)phenyl, 4-(acetylamino)phenyl, 3-(propionylamino)phenyl, 4-(2-oxo-pyrrolidin-1-yl)phenyl, 3-[(methylsulfonyl)amino]phenyl, 3-hydroxyphenyl, 3-methoxyphenyl, 4-methoxyphenyl, 4-difluoromethoxyphenyl, 4-trifluoromethoxyphenyl, 3-ethoxyphenyl, 3-(2,2,2-trifluoroethoxy)phenyl, 4-isopropoxyphenyl, 3-(carboxymethyloxy)phenyl, 3-[(isopropoxycarbonyl)methyloxy]phenyl, 3-[(dimethylaminocarbonyl)methyloxy]phenyl, 4-(methoxyethyloxy)phenyl, 4-(dimethylaminoethyloxy)phenyl, 4-(diethylaminoethyloxy)phenyl, 4-[(morpholin-4-yl)ethyloxy]phenyl, 3-fluoro-4-methoxyphenyl, 3-chloro-4-hydroxyphenyl, 3-chloro-4-methoxyphenyl, 4-chloro-3-methoxyphenyl, 3-methoxy-5-trifluoromethylphenyl, 4-methoxy-3-trifluoromethylphenyl, 3,4-dimethoxyphenyl, 3,5-dimethoxyphenyl, 3,5-dichloro-4-hydroxyphenyl, 2,3,4-trimethoxyphenyl, 3,4,5-trimethoxyphenyl, 4-(methylthio)phenyl, 4-(trifluoromethylthio)phenyl, 3-methylsulfonylphenyl, 4-methylsulfonylphenyl, 3-aminosulfonylphenyl, 3-(methylamino)sulfonylphenyl, 4-(methylamino)sulfonylphenyl, 3-(ethylamino)sulfonylphenyl, 3-(isopropylamino)sulfonylphenyl, 3-(dimethylamino)sulfonylphenyl, or 3-(morpholin-4-yl)sulfonylphenyl.

Suitably, R⁷ is (C₁-C₄)alkyl, aryl, heterocycloalkyl, or heterocycloalkyl(C₁-C₂)alkyl, wherein said (C₁-C₄)alkyl is optionally substituted one to three times, independently, by halogen, hydroxyl, (C₁-C₄)alkoxy, amino, (C₁-C₄)alkylamino, ((C₁-C₄)alkyl)((C₁-C₄)alkyl)amino, —CO₂H, —CO₂(C₁-C₄)alkyl, —CONH₂, —CONH(C₁-C₄)alkyl, or —CON((C₁-C₄)alkyl)((C₁-C₄)alkyl); and wherein any heterocycloalkyl is optionally substituted by (C₁-C₄)alkyl. In another embodiment of this invention, R⁷ is (C₁-C₄)alkyl, phenyl, pyrrolidinyl, piperidinyl, morpholinyl, thiomorpholinyl, piperazinyl, or pyrrolidinyl(C₁-C₂)alkyl, piperidinyl(C₁-C₂)alkyl, morpholinyl(C₁-C₂)alkyl, thiomorpholinyl(C₁-C₂)alkyl, or piperazinyl(C₁-C₂)alkyl, wherein said (C₁-C₄)alkyl is optionally substituted one to three times, independently, by halogen, hydroxyl, (C₁-C₄)alkoxy, amino, (C₁-C₄)alkylamino, ((C₁-C₄)alkyl)((C₁-C₄)alkyl)amino, —CO₂H, —CO₂(C₁-C₄)alkyl, —CONH₂, —CONH(C₁-C₄)alkyl, or —CON((C₁-C₄)alkyl)((C₁-C₄)alkyl); and wherein any pyrrolidinyl, piperidinyl, morpholinyl, thiomorpholinyl, or piperazinyl is optionally substituted by (C₁-C₄)alkyl. In a specific embodiment of this invention, R⁷ is methyl, difluoromethyl, trifluoromethyl, ethyl, 2,2,2-trifluoroethyl, isopropyl, dimethylaminoethyl, diethylaminoethyl, hydroxyethyl, methoxyethyl, methoxypropyl, carboxymethyl, (isopropoxycarbonyl)methyl, (dimethylaminocarbonyl)methyl, phenyl, 1-methyl-piperidin-4-yl, or (morpholin-4-yl)ethyl.

Suitably, R⁸ is (C₁-C₄)alkyl. In a specific embodiment of this invention, R⁸ is methyl or ethyl.

In another embodiment of this invention, R⁷ and R⁸ taken together with the nitrogen to which they are attached represent a 5-7 membered heterocyclic ring, optionally containing an additional heteroatom selected from oxygen, nitrogen, and sulfur, wherein said ring is optionally substituted one or two times, independently, by halogen, (C₁-C₄)alkyl, (C₁-C₄)haloalkyl, amino, (C₁-C₄)alkylamino, ((C₁-C₄)alkyl)((C₁-C₄)alkyl)amino, hydroxyl, oxo, (C₁-C₄)alkoxy, or (C₁-C₄)alkoxy(C₁-C₄)alkyl. In yet another embodiment of this invention, R⁷ and R⁸ taken together with the nitrogen to which they are attached represent pyrrolidinyl, piperidinyl, morpholinyl, thiomorpholinyl, piperazinyl, or hexahydro-1H-1,4-diazepinyl, each optionally substituted one or two times, independently, by halogen, (C₁-C₄)alkyl, (C₁-C₄)haloalkyl, amino, (C₁-C₄)alkylamino, ((C₁-C₄)alkyl)((C₁-C₄)alkyl)amino, hydroxyl, oxo, (C₁-C₄)alkoxy, or (C₁-C₄)alkoxy(C₁-C₄)alkyl. In a specific embodiment of this invention, R⁷ and R⁸ taken together with the nitrogen to which they are attached represent pyrrolidinyl, 2-methylpyrrolidinyl, 2-trifluoromethylpyrrolidinyl, 3-(dimethylamino)pyrrolidinyl, 2-oxo-pyrrolidinyl, 2,5-dimethylpyrrolidinyl, 3,3-difluoropyrrolidinyl, piperidinyl, 3,3-difluoropiperidinyl, 4,4-difluoropiperidinyl, morpholinyl, thiomorpholinyl, piperazinyl, 4-methylpiperazinyl, 4-methoxyethylpiperazinyl, or 4-methyl-hexahydro-1H-1,4-diazepinyl.

One particular embodiment of the invention is a compound of Formula I or a salt thereof wherein:

R¹ is (C₁-C₄)alkyl;

R² is hydrogen;

R³ is hydrogen, halogen, (C₁-C₄)alkyl, (C₁-C₄)haloalkyl, (C₃-C₆)cycloalkyl, aryl, hydroxyl, hydroxy(C₁-C₄)alkyl-, (C₁-C₄)alkoxy, (C₁-C₄)alkoxy(C₁-C₄)alkyl-, (C₁-C₄)haloalkoxy, (C₃-C₆)cycloalkyloxy, (C₁-C₄)alkylthio-, amino, (C₁-C₄)alkylamino, or ((C₁-C₄)alkyl)((C₁-C₄)alkyl)amino;

R⁴ is hydrogen, halogen, (C₁-C₈)alkyl, (C₁-C₈)haloalkyl, (C₃-C₈)cycloalkyl, hydroxyl, hydroxy(C₁-C₈)alkyl-, (C₁-C₈)alkoxy, (C₁-C₄)alkoxy(C₁-C₈)alkyl-, (C₁-C₈)haloalkoxy, (C₃-C₈)cycloalkyloxy, (C₁-C₈)alkylthio-, —SO₂(C₁-C₄)alkyl, or —NR⁷R⁸;

R⁵ is hydrogen;

or R⁴ and R⁵ taken together with atoms through which they are connected form a partially saturated 5 or 6 membered ring, optionally containing one or two additional heteroatoms selected from N, O and S, which ring may be unsubstituted or substituted with one to three substituents independently selected from (C₁-C₄)alkyl, (C₁-C₄)haloalkyl, hydroxy(C₁-C₄)alkyl-, (C₁-C₄)alkoxy, (C₁-C₄)haloalkoxy, and (C₁-C₄)alkylthio-;

R⁶ is (C₁-C₈)alkyl, (C₂-C₈)alkenyl, (C₂-C₈)alkynyl, (C₃-C₈)cycloalkyl, aryl, or heteroaryl, wherein any aryl or heteroaryl group is optionally substituted one to three times, independently, by halogen, (C₁-C₆)alkyl, (C₃-C₆)cycloalkyl, (C₁-C₄)haloalkyl, cyano, —CO(C₁-C₄)alkyl, —CO₂H, —CO₂R⁷, —CONH₂, —CONHR⁷, —CONR⁷R⁸, HO₂C(C₁-C₂)alkyl-, R⁷O₂C(C₁-C₂)alkyl-, —SR⁷, —SO₂(C₁-C₄)alkyl, —SO₂NH₂, —SO₂NHR⁷, —SO₂NR⁷R⁸, nitro, amino, —NHR⁷, —NR⁷R⁸, amino(C₁-C₂)alkyl-, R⁷HN(C₁-C₂)alkyl-, R⁷R⁸N(C₁-C₂)alkyl-, —NHCO(C₁-C₄)alkyl, —NHSO₂(C₁-C₄)alkyl, oxo, hydroxyl, —OR⁷, hydroxy(C₁-C₂)alkyl-, R⁷O(C₁-C₂)alkyl-, aryl, or heteroaryl, wherein said aryl or heteroaryl is optionally substituted one to three times, independently, by halogen, (C₁-C₆)alkyl, (C₃-C₆)cycloalkyl, (C₁-C₄)haloalkyl, cyano, —CO(C₁-C₄)alkyl, —CO₂H, —CO₂R⁷, —CONH₂, —CONHR⁷, —CONR⁷R⁸, —SR⁷, —SO₂(C₁-C₄)alkyl, —SO₂NH₂, —SO₂NHR⁷, —SO₂NR⁷R⁸, nitro, amino, —NHR⁷, —NR⁷R⁸, —NHCO(C₁-C₄)alkyl, —NHSO₂(C₁-C₄)alkyl, oxo, hydroxyl, —OR⁷, hydroxy(C₁-C₂)alkyl-, or R⁷O(C₁-C₂)alkyl-;

R⁷ is (C₁-C₄)alkyl, aryl, heterocycloalkyl, or heterocycloalkyl(C₁-C₂)alkyl, wherein said (C₁-C₄)alkyl is optionally substituted one to three times, independently, by halogen, hydroxyl, (C₁-C₄)alkoxy, amino, (C₁-C₄)alkylamino, ((C₁-C₄)alkyl)((C₁-C₄)alkyl)amino, —CO₂H, —CO₂(C₁-C₄)alkyl, —CONH₂, —CONH(C₁-C₄)alkyl, or —CON((C₁-C₄)alkyl)((C₁-C₄)alkyl); and wherein any heterocycloalkyl is optionally substituted by (C₁-C₄)alkyl; and

R⁸ is (C₁-C₄)alkyl;

or R⁷ and R⁸ taken together with the nitrogen to which they are attached represent a 5-7 membered heterocyclic ring, optionally containing an additional heteroatom selected from oxygen, nitrogen, and sulfur, wherein said ring is optionally substituted one or two times, independently, by halogen, (C₁-C₄)alkyl, (C₁-C₄)haloalkyl, amino, (C₁-C₄)alkylamino, ((C₁-C₄)alkyl)((C₁-C₄)alkyl)amino, hydroxyl, oxo, (C₁-C₄)alkoxy, or (C₁-C₄)alkoxy(C₁-C₄)alkyl.

Another particular embodiment of the invention is a compound of Formula I or a salt thereof wherein:

R¹ is methyl;

R² is hydrogen or fluorine;

R³ is hydrogen, halogen, (C₁-C₄)alkyl, (C₁-C₄)haloalkyl, phenyl, (C₁-C₄)alkoxy, (C₁-C₄)alkylthio-, or ((C₁-C₄)alkyl)((C₁-C₄)alkyl)amino; R⁴ is hydrogen, halogen, (C₁-C₈)alkyl, (C₁-C₈)haloalkyl, (C₃-C₈)cycloalkyl, hydroxyl, hydroxy(C₁-C₈)alkyl-, (C₁-C₈)alkoxy, (C₁-C₄)alkoxy(C₁-C₈)alkyl-, (C₁-C₈)haloalkoxy, (C₃-C₈)cycloalkyloxy, (C₁-C₈)alkylthio-, (C₁-C₈)haloalkylthio-, —SO₂(C₁-C₄)alkyl, amino, (C₁-C₄)alkylamino, (C₁-C₄)haloalkylamino, ((C₁-C₄)alkyl)((C₁-C₄)alkyl)amino, ((C₁-C₄)alkyl)((C₁-C₄)haloalkyl)amino, ((C₁-C₄)haloalkyl)((C₁-C₄)haloalkyl)amino, pyrrolidinyl, imidazolidinyl, pyrazolidinyl, piperidinyl, piperazinyl, morpholinyl, or thiomorpholinyl, wherein said pyrrolidinyl, imidazolidinyl, pyrazolidinyl, piperidinyl, piperazinyl, morpholinyl, or thiomorpholinyl is optionally substituted one or two times, independently, by halogen, (C₁-C₄)alkyl, (C₁-C₄)haloalkyl, amino, (C₁-C₄)alkylamino, ((C₁-C₄)alkyl)((C₁-C₄)alkyl)amino, hydroxyl, oxo, (C₁-C₄)alkoxy, or (C₁-C₄)alkoxy(C₁-C₄)alkyl;

R⁵ is hydrogen;

R⁶ is phenyl optionally substituted one to three times, independently, by halogen, (C₁-C₆)alkyl, (C₂-C₆)alkenyl, (C₂-C₆)alkynyl, (C₃-C₆)cycloalkyl, (C₁-C₄)haloalkyl, cyano, —CO(C₁-C₄)alkyl, —CO₂H, —CO₂R⁷, —CONH₂, —CONHR⁷, —CONR⁷R⁸, HO₂C(C₁-C₂)alkyl-, R⁷O₂C(C₁-C₂)alkyl-, cyano(C₁-C₂)alkyl-, —SR⁷, —SO₂(C₁-C₄)alkyl, —SO₂NH₂, —SO₂NHR⁷, —SO₂NR⁷R⁸, nitro, amino, —NHR⁷, —NR⁷R⁸, amino(C₁-C₂)alkyl-, R⁷HN(C₁-C₂)alkyl-, R⁷R⁸N(C₁-C₂)alkyl-, triazolyl(C₁-C₂)alkyl-, —NHCO(C₁-C₄)alkyl, —NHSO₂(C₁-C₄)alkyl, oxo, hydroxyl, —OR⁷, hydroxy(C₁-C₂)alkyl-, R⁷O(C₁-C₂)alkyl-, phenyl, thienyl, pyrazolyl, imidazolyl, oxazolyl, thiazolyl, or pyridinyl, wherein said phenyl, thienyl, pyrazolyl, imidazolyl, oxazolyl, thiazolyl, or pyridinyl is optionally substituted one or two times, independently, by halogen, (C₁-C₆)alkyl, (C₃-C₆)cycloalkyl, (C₁-C₄)haloalkyl, cyano, —CO(C₁-C₄)alkyl, —CO₂H, —CO₂R⁷, —CONH₂, —CONHR⁷, —CONR⁷R⁸, —SR⁷, —SO₂(C₁-C₄)alkyl, —SO₂NH₂, —SO₂NHR⁷, —SO₂NR⁷R⁸, nitro, amino, —NHR⁷, —NR⁷R⁸, —NHCO(C₁-C₄)alkyl, —NHSO₂(C₁-C₄)alkyl, oxo, hydroxyl, —OR⁷, hydroxy(C₁-C₂)alkyl-, or R⁷O(C₁-C₂)alkyl-;

R⁷ is (C₁-C₄)alkyl, phenyl, pyrrolidinyl, piperidinyl, morpholinyl, thiomorpholinyl, piperazinyl, or pyrrolidinyl(C₁-C₂)alkyl, piperidinyl(C₁-C₂)alkyl, morpholinyl(C₁-C₂)alkyl, thiomorpholinyl(C₁-C₂)alkyl, or piperazinyl(C₁-C₂)alkyl, wherein said (C₁-C₄)alkyl is optionally substituted one to three times, independently, by halogen, hydroxyl, (C₁-C₄)alkoxy, amino, (C₁-C₄)alkylamino, ((C₁-C₄)alkyl)((C₁-C₄)alkyl)amino, —CO₂H, —CO₂(C₁-C₄)alkyl, —CONH₂, —CONH(C₁-C₄)alkyl, or —CON((C₁-C₄)alkyl)((C₁-C₄)alkyl); and wherein any pyrrolidinyl, piperidinyl, morpholinyl, thiomorpholinyl, or piperazinyl is optionally substituted by (C₁-C₄)alkyl; and

R⁸ is methyl or ethyl;

or R⁷ and R⁸ taken together with the nitrogen to which they are attached represent pyrrolidinyl, piperidinyl, morpholinyl, thiomorpholinyl, piperazinyl, or hexahydro-1H-1,4-diazepinyl, each optionally substituted one or two times, independently, by halogen, (C₁-C₄)alkyl, (C₁-C₄)haloalkyl, amino, (C₁-C₄)alkylamino, ((C₁-C₄)alkyl)((C₁-C₄)alkyl)amino, hydroxyl, oxo, (C₁-C₄)alkoxy, or (C₁-C₄)alkoxy(C₁-C₄)alkyl.

Another particular embodiment of the invention is a compound of Formula I or a salt thereof wherein:

R¹ is methyl;

R² is hydrogen or fluorine;

R³ is hydrogen, halogen, (C₁-C₄)alkyl, (C₁-C₄)haloalkyl, phenyl, (C₁-C₄)alkoxy, (C₁-C₄)alkylthio-, or ((C₁-C₄)alkyl)((C₁-C₄)alkyl)amino;

R⁴ is hydrogen, halogen, (C₁-C₈)alkyl, (C₁-C₈)haloalkyl, (C₃-C₈)cycloalkyl, hydroxyl, hydroxy(C₁-C₈)alkyl-, (C₁-C₈)alkoxy, (C₁-C₄)alkoxy(C₁-C₈)alkyl-, (C₁-C₈)haloalkoxy, (C₃-C₈)cycloalkyloxy, (C₁-C₈)alkylthio-, (C₁-C₈)haloalkylthio-, —SO₂(C₁-C₄)alkyl, amino, (C₁-C₄)alkylamino, (C₁-C₄)haloalkylamino, ((C₁-C₄)alkyl)((C₁-C₄)alkyl)amino, ((C₁-C₄)alkyl)((C₁-C₄)haloalkyl)amino, ((C₁-C₄)haloalkyl)((C₁-C₄)haloalkyl)amino, pyrrolidinyl, imidazolidinyl, pyrazolidinyl, piperidinyl, piperazinyl, morpholinyl, or thiomorpholinyl, wherein said pyrrolidinyl, imidazolidinyl, pyrazolidinyl, piperidinyl, piperazinyl, morpholinyl, or thiomorpholinyl is optionally substituted one or two times, independently, by halogen, (C₁-C₄)alkyl, (C₁-C₄)haloalkyl, amino, (C₁-C₄)alkylamino, ((C₁-C₄)alkyl)((C₁-C₄)alkyl)amino, hydroxyl, oxo, (C₁-C₄)alkoxy, or (C₁-C₄)alkoxy(C₁-C₄)alkyl;

R⁵ is hydrogen;

R⁶ is pyridinyl optionally substituted one or two times, independently, by halogen, (C₁-C₆)alkyl, (C₃-C₆)cycloalkyl, (C₁-C₄)haloalkyl, cyano, —CO(C₁-C₄)alkyl, —CO₂H, —CO₂R⁷, —CONH₂, —CONHR⁷, —CONR⁷R⁸, HO₂C(C₁-C₂)alkyl-, R⁷O₂C(C₁-C₂)alkyl-, —SR⁷, —SO₂(C₁-C₄)alkyl, —SO₂NH₂, —SO₂NHR⁷, —SO₂NR⁷R⁸, nitro, amino, —NHR⁷, —NR⁷R⁸, amino(C₁-C₂)alkyl-, R⁷HN(C₁-C₂)alkyl-, R⁷R⁸N(C₁-C₂)alkyl-, —NHCO(C₁-C₄)alkyl, —NHSO₂(C₁-C₄)alkyl, oxo, hydroxyl, —OR⁷, hydroxy(C₁-C₂)alkyl-, or R⁷O(C₁-C₂)alkyl-;

R⁷ is (C₁-C₄)alkyl, phenyl, pyrrolidinyl, piperidinyl, morpholinyl, thiomorpholinyl, piperazinyl, or pyrrolidinyl(C₁-C₂)alkyl, piperidinyl(C₁-C₂)alkyl, morpholinyl(C₁-C₂)alkyl, thiomorpholinyl(C₁-C₂)alkyl, or piperazinyl(C₁-C₂)alkyl, wherein said (C₁-C₄)alkyl is optionally substituted one to three times, independently, by halogen, hydroxyl, (C₁-C₄)alkoxy, amino, (C₁-C₄)alkylamino, ((C₁-C₄)alkyl)((C₁-C₄)alkyl)amino, —CO₂H, —CO₂(C₁-C₄)alkyl, —CONH₂, —CONH(C₁-C₄)alkyl, or —CON((C₁-C₄)alkyl)((C₁-C₄)alkyl); and wherein any pyrrolidinyl, piperidinyl, morpholinyl, thiomorpholinyl, or piperazinyl is optionally substituted by (C₁-C₄)alkyl; and

R⁸ is methyl or ethyl;

or R⁷ and R⁸ taken together with the nitrogen to which they are attached represent pyrrolidinyl, piperidinyl, morpholinyl, thiomorpholinyl, piperazinyl, or hexahydro-1H-1,4-diazepinyl, each optionally substituted one or two times, independently, by halogen, (C₁-C₄)alkyl, (C₁-C₄)haloalkyl, amino, (C₁-C₄)alkylamino, ((C₁-C₄)alkyl)((C₁-C₄)alkyl)amino, hydroxyl, oxo, (C₁-C₄)alkoxy, or (C₁-C₄)alkoxy(C₁-C₄)alkyl.

Specific compounds of this invention include:
N-methyl-3-({6-[(3-methylphenyl)amino]-4-pyrimidinyl}amino)benzenesulfonamide;
3-({6-[(3-chlorophenyl)amino]-4-pyrimidinyl}amino)-N-methylbenzenesulfonamide;
N-methyl-3-{[6-(methylamino)-4-pyrimidinyl]amino}benzenesulfonamide;
3-{[6-(ethylamino)-4-pyrimidinyl]amino}-N-methylbenzenesulfonamide;
3,3′-(4,6-pyrimidinediyldiimino)bis(N-methylbenzenesulfonamide);
3-({6-[(4-chlorophenyl)amino]-4-pyrimidinyl}amino)-5-(dimethylamino)-N-methylbenzenesulfonamide;
3-chloro-5-({6-[(4-chlorophenyl)amino]-4-pyrimidinyl}amino)-N-methylbenzenesulfonamide;
3-({6-[(4-chlorophenyl)amino]-4-pyrimidinyl}amino)-N-methyl-4-(propyloxy)benzenesulfonamide;
3-({6-[(4-chlorophenyl)amino]-4-pyrimidinyl}amino)-4-(ethyloxy)-N-methylbenzenesulfonamide;
3-({6-[(4-chlorophenyl)amino]-4-pyrimidinyl}amino)-N-methyl-4-[(2-methylpropyl)oxy]benzenesulfonamide;
3-({6-[(4-chlorophenyl)amino]-4-pyrimidinyl}amino)-4-[(1,2-dimethylpropyl)oxy]-N-methylbenzenesulfonamide;
4-chloro-3-({6-[(4-chlorophenyl)amino]-4-pyrimidinyl}amino)-N-methylbenzenesulfonamide;
3-({6-[(4-chlorophenyl)amino]-4-pyrimidinyl}amino)-N-methyl-4-[(2,2,2-trifluoroethyl)oxy]-benzenesulfonamide;
3-({6-[(4-chlorophenyl)amino]-4-pyrimidinyl}amino)-4-(cyclohexyloxy)-N-methylbenzenesulfonamide;
3-({6-[(4-chlorophenyl)amino]-4-pyrimidinyl}amino)-4-[(1-ethylpropyl)oxy]-N-methylbenzenesulfonamide;
3-({6-[(4-chlorophenyl)amino]-4-pyrimidinyl}amino)-N-methyl-4-[(3,3,3-trifluoropropyl)oxy]-benzenesulfonamide;
3-({6-[(4-chlorophenyl)amino]-4-pyrimidinyl}amino)-4-(cyclopentyloxy)-N-methylbenzenesulfonamide;
5-(6-(4-chlorophenylamino)pyrimidin-4-ylamino)-2-fluoro-4-methoxy-N-methylbenzenesulfonamide;
3-({6-[(4-chlorophenyl)amino]-4-pyrimidinyl}amino)-N-methyl-4-[methyl(2,2,2-trifluoroethyl)amino]benzenesulfonamide;
1-[6-(4-chloro-phenylamino)-pyrimidin-4-yl]-3,3-dimethyl-2,3-dihydro-1H-indole-6-sulfonic acid methylamide;
3-({6-[(4-chlorophenyl)amino]-4-pyrimidinyl}amino)-N-methyl-4-[(2,2,2-trifluoro-1-methylethyl)oxy]benzenesulfonamide;
5-(6-(4-chlorophenylamino)pyrimidin-4-ylamino)-2-fluoro-N-methyl-4-(2,2,2-trifluoroethoxy)benzenesulfonamide;
4-amino-3-({6-[(4-chlorophenyl)amino]-4-pyrimidinyl}amino)-N-methylbenzenesulfonamide;
5-[6-(4-chloro-phenylamino)-pyrimidin-4-ylamino]-4-dimethylamino-2-fluoro-N-methyl-benzenesulfonamide;
3-({6-[(4-chlorophenyl)amino]-4-pyrimidinyl}amino)-4-(3,3-difluoro-1-piperidinyl)-N-methylbenzenesulfonamide;
3-({6-[(4-chlorophenyl)amino]-4-pyrimidinyl}amino)-N-methyl-4-{[2,2,2-trifluoro-1-(trifluoromethyl)ethyl]oxy}benzenesulfonamide;
4-(dimethylamino)-3-({6-[(3-fluorophenyl)amino]-4-pyrimidinyl}amino)-N-methylbenzenesulfonamide;
3-({6-[(3-fluorophenyl)amino]-4-pyrimidinyl}amino)-N-methyl-4-(4-morpholinyl)benzenesulfonamide;
1-{6-[(3-fluorophenyl)amino]-4-pyrimidinyl}-N-methyl-2,3-dihydro-1H-indole-6-sulfonamide;
3-({6-[(3-fluorophenyl)amino]-4-pyrimidinyl}amino)-N-methyl-4-(methyloxy)benzenesulfonamide;
N-methyl-3-[(6-{[4-(1-methylethyl)phenyl]amino}-4-pyrimidinyl)amino]-4-(methylthio)benzenesulfonamide;
3-[(6-{[3-chloro-4-(methyloxy)phenyl]amino}-4-pyrimidinyl)amino]-N-methyl-4-[(2,2,2-trifluoroethyl)oxy]benzenesulfonamide;
3-[(6-{[3-chloro-4-(methyloxy)phenyl]amino}-4-pyrimidinyl)amino]-N-methyl-4-(methyloxy)benzenesulfonamide;
N-methyl-4-(methyloxy)-3-({6-[(4-{[2-(methyloxy)ethyl]oxy}phenyl)amino]-4-pyrimidinyl}amino)benzenesulfonamide;
N-methyl-3-({6-[(4-{[2-(methyloxy)ethyl]oxy}phenyl)amino]-4-pyrimidinyl}amino)-4-[(2,2,2-trifluoroethyl)oxy]benzenesulfonamide;
N-methyl-4-(methyloxy)-3-[(6-{[4-(2,2,2-trifluoroethyl)phenyl]amino}-4-pyrimidinyl)amino]benzenesulfonamide;
N-methyl-4-[(2,2,2-trifluoroethyl)oxy]-3-[(6-{[4-(2,2,2-trifluoroethyl)phenyl]amino}-4-pyrimidinyl)amino]benzenesulfonamide;
N-methyl-3-[(6-{[4-(2,2,2-trifluoroethyl)phenyl]amino}-4-pyrimidinyl)amino]-4-[(2,2,2-trifluoroethyl)thio]benzenesulfonamide;
4-[(6-{[5-[(methylamino)sulfonyl]-2-(methylthio)phenyl]amino}-4-pyrimidinyl)amino]-N-[2-(methyloxy)ethyl]benzamide;
N-methyl-4-(methyloxy)-3-[(6-{[4-(1H-pyrazol-1-yl)phenyl]amino}-4-pyrimidinyl)amino]benzenesulfonamide;
N-methyl-3-[(6-{[4-(1H-pyrazol-1-yl)phenyl]amino}-4-pyrimidinyl)amino]-4-[(2,2,2-trifluoroethyl)oxy]benzenesulfonamide;
N-methyl-4-[(2,2,2-trifluoroethyl)oxy]-3-{[6-({4-[(2,2,2-trifluoroethyl)oxy]phenyl}amino)-4-pyrimidinyl]amino}benzenesulfonamide;
N-methyl-4-[(2,2,2-trifluoroethyl)oxy]-3-[(6-{[4-(trifluoromethyl)phenyl]amino}-4-pyrimidinyl)amino]benzenesulfonamide;
3-({6-[(3,4-difluorophenyl)amino]-4-pyrimidinyl}amino)-4-fluoro-N-methylbenzenesulfonamide;
3-({4-[(3,4-difluorophenyl)amino]-4-pyrimidinyl}amino)-N-methyl-4-[(2,2,2-trifluoro-1-methylethyl)oxy]benzenesulfonamide;
1-{6-[(3,4-difluorophenyl)amino]-4-pyrimidinyl}-N,3,3-trimethyl-2,3-dihydro-1H-indole-6-sulfonamide;
3-[6-(6-bromo-4-methyl-pyridin-2-ylamino)-pyrimidin-4-ylamino]-N-methyl-4-(2,2,2-trifluoro-ethoxy)-benzenesulfonamide;
3-({6-[(3,5-dichloro-2-pyridinyl)amino]-4-pyrimidinyl}amino)-N-methyl-4-[(2,2,2-trifluoroethyl)oxy]benzenesulfonamide;
3-{[6-(3-biphenylylamino)-4-pyrimidinyl]amino}-N-methylbenzenesulfonamide;
N-methyl-3-({6-[(4-methylphenyl)amino]-4-pyrimidinyl}amino)benzenesulfonamide;
3-{[6-({3-[(methylamino)sulfonyl]phenyl}amino)-4-pyrimidinyl]amino}benzamide;
3-({4-[(3-acetylphenyl)amino]-4-pyrimidinyl}amino)-N-methylbenzenesulfonamide;
N-methyl-3-[(6-{[3-(methyloxy)phenyl]amino}-4-pyrimidinyl)amino]benzenesulfonamide;
N-(3-{[6-({3-[(methylamino)sulfonyl]phenyl}amino)-4-pyrimidinyl]amino}phenyl)acetamide;
N-methyl-3-{[6-(phenylamino)-4-pyrimidinyl]amino}benzenesulfonamide;
4-{[6-({3-[(methylamino)sulfonyl]phenyl}amino)-4-pyrimidinyl]amino}benzamide;
3-({6-[(4-chlorophenyl)amino]-4-pyrimidinyl}amino)-N-methylbenzenesulfonamide;
N-methyl-3-[(6-{[3-(trifluoromethyl)phenyl]amino}-4-pyrimidinyl)amino]benzenesulfonamide;
N-methyl-3-({6-[(2-methyl-1,2,3,4-tetrahydro-7-isoquinolinyl)amino]-4-pyrimidinyl}amino)benzenesulfonamide;
3-({4-[(2-fluorophenyl)amino]-4-pyrimidinyl}amino)-N-methylbenzenesulfonamide;
N-methyl-3-[(6-{[3-(4-morpholinylsulfonyl)phenyl]amino}-4-pyrimidinyl)amino]benzenesulfonamide;
3-{[6-({3-[(ethylamino)sulfonyl]phenyl}amino)-4-pyrimidinyl]amino}-N-methylbenzenesulfonamide;
N-methyl-3-[(6-{[3-(methylsulfonyl)phenyl]amino}-4-pyrimidinyl)amino]benzenesulfonamide;
3-[6-(1H-indazol-6-ylamino)-pyrimidin-4-ylamino]-N-methyl-benzenesulfonamide;
3-{[6-({3-[(methylamino)sulfonyl]phenyl}amino)-4-pyrimidinyl]amino}-N-phenylbenzamide;
3-{[6-({3-[(dimethylamino)sulfonyl]phenyl}amino)-4-pyrimidinyl]amino}-N-methylbenzenesulfonamide;
3-[(6-{[3-(aminosulfonyl)phenyl]amino}-4-pyrimidinyl)amino]-N-methylbenzenesulfonamide;
3-{[6-({3-[(methylamino)sulfonyl]phenyl}amino)-4-pyrimidinyl]amino}-N-(1-methylethyl)benzenesulfonamide;
3-({6-[(4-acetylphenyl)amino]-4-pyrimidinyl}amino)-N-methylbenzenesulfonamide;
N-methyl-3-[(6-{[4-(methylsulfonyl)phenyl]amino}-4-pyrimidinyl)amino]benzenesulfonamide;
N-(4-{[6-({3-[(methylamino)sulfonyl]phenyl}amino)-4-pyrimidinyl]amino}phenyl)acetamide;
N-(3-{[6-({3-[(methylamino)sulfonyl]phenyl}amino)-4-pyrimidinyl]amino}phenyl)propanamide;
4-{[6-({3-[(methylamino)sulfonyl]phenyl}amino)-4-pyrimidinyl]amino}-N-phenylbenzamide;
3-({6-[(1,1-dioxido-2,3-dihydro-1,2-benzisothiazol-6-yl)amino]-4-pyrimidinyl}amino)-N-methylbenzenesulfonamide;
N-methyl-3-({6-[(2-oxo-2,3-dihydro-1H-indol-6-yl)amino]-4-pyrimidinyl}amino)benzenesulfonamide;
N-methyl-3-[6-(2-methyl-benzothiazol-5-ylamino)-pyrimidin-4-ylamino]-benzenesulfonamide;
N-methyl-3-({6-[(3-nitrophenyl)amino]-4-pyrimidinyl}amino)benzenesulfonamide;
N-methyl-3-[(6-{[4-(4-morpholinylcarbonyl)phenyl]amino}-4-pyrimidinyl)amino]benzenesulfonamide;
N-methyl-4-{[6-({3-[(methylamino)sulfonyl]phenyl}amino)-4-pyrimidinyl]amino}benzamide;
3-[6-(2,3-dihydro-benzo[1,4]dioxin-6-ylamino)-pyrimidin-4-ylamino]-N-methyl-benzenesulfonamide;
N-methyl-3-[(6-{[4-(methyloxy)phenyl]amino}-4-pyrimidinyl)amino]benzenesulfonamide;
N-methyl-3-[(6-{[4-(4-morpholinyl)phenyl]amino}-4-pyrimidinyl)amino]benzenesulfonamide;
3-[(6-{[4-(1,1-dimethylethyl)phenyl]amino}-4-pyrimidinyl)amino]-N-methylbenzenesulfonamide;
N-methyl-3-[(6-{[3-(4-morpholinyl)phenyl]amino}-4-pyrimidinyl)amino]benzenesulfonamide;
3-({6-[(3-bromo-5-methylphenyl)amino]-4-pyrimidinyl}amino)-N-methylbenzenesulfonamide;
3-[(6-{[4-(dimethylamino)phenyl]amino}-4-pyrimidinyl)amino]-N-methylbenzenesulfonamide;
3-[(6-{[3-(dimethylamino)phenyl]amino}-4-pyrimidinyl)amino]-N-methylbenzenesulfonamide;
methyl 4-{[6-({3-[(methylamino)sulfonyl]phenyl}amino)-4-pyrimidinyl]amino}benzoate;
1-methylethyl 4-{[6-({3-[(methylamino)sulfonyl]phenyl}amino)-4-pyrimidinyl]amino}benzoate;
3-({6-[(4-chloro-3-methylphenyl)amino]-4-pyrimidinyl}amino)-N-methylbenzenesulfonamide;
3-({6-[(4-fluoro-3-methylphenyl)amino]-4-pyrimidinyl}amino)-N-methylbenzenesulfonamide;
3-{[6-(1H-indol-6-ylamino)-4-pyrimidinyl]amino}-N-methylbenzenesulfonamide;
N-methyl-3-{[6-({3-[(methylsulfonyl)amino]phenyl}amino)-4-pyrimidinyl]amino}benzenesulfonamide;
N-methyl-3-({6-[(3-methyl-1H-indazol-6-yl)amino]-4-pyrimidinyl}amino)benzenesulfonamide;
3-({6-[(4-{[2-(diethylamino)ethyl]oxy}phenyl)amino]-4-pyrimidinyl}amino)-N-methylbenzenesulfonamide;
1-methylethyl[(3-{[6-({3-[(methylamino)sulfonyl]phenyl}amino)-4-pyrimidinyl]amino}phenyl)oxy]acetate;
3-[6-(benzothiazol-6-ylamino)-pyrimidin-4-ylamino]-N-methyl-benzenesulfonamide;
3-[6-(1H-indol-5-ylamino)-pyrimidin-4-ylamino]-N-methyl-benzenesulfonamide;
3-{[6-(1,3-benzothiazol-5-ylamino)-4-pyrimidinyl]amino}-N-methylbenzenesulfonamide;
3-({6-[(3-fluoro-4-methylphenyl)amino]-4-pyrimidinyl}amino)-N-methylbenzenesulfonamide;
3-({6-[(3-fluorophenyl)amino]-4-pyrimidinyl}amino)-N-methylbenzenesulfonamide;
3-[(6-{[3-fluoro-4-(trifluoromethyl)phenyl]amino}-4-pyrimidinyl)amino]-N-methylbenzenesulfonamide;
N-methyl-3-[(6-{[4-(methyloxy)-3-(trifluoromethyl)phenyl]amino}-4-pyrimidinyl)amino]benzenesulfonamide;
3-({6-[(4-chloro-3-fluorophenyl)amino]-4-pyrimidinyl}amino)-N-methylbenzenesulfonamide;
3-[(6-{[3-fluoro-4-(methyloxy)phenyl]amino}-4-pyrimidinyl)amino]-N-methylbenzenesulfonamide;
N-methyl-3-[(6-{[4-methyl-3-(trifluoromethyl)phenyl]amino}-4-pyrimidinyl)amino]benzenesulfonamide;
3-[(6-{[4-chloro-3-(trifluoromethyl)phenyl]amino}-4-pyrimidinyl)amino]-N-methylbenzenesulfonamide;
N-methyl-3-[(6-{[4-(2,2,2-trifluoroethyl)phenyl]amino}-4-pyrimidinyl)amino]benzenesulfonamide;
N-methyl-4-(methylthio)-3-({6-[(2-oxo-1,2,3,4-tetrahydro-7-quinolinyl)amino]-4-pyrimidinyl}amino)benzenesulfonamide;
4-[(6-{[5-[(methylamino)sulfonyl]-2-(methylthio)phenyl]amino}-4-pyrimidinyl)amino]benzoic acid;
3-({6-[(4-chlorophenyl)amino]-4-pyrimidinyl}amino)-4-(diethylamino)-N-methylbenzenesulfonamide;
3-({6-[(4-chlorophenyl)amino]-4-pyrimidinyl}amino)-4-(2,5-dimethyl-1-pyrrolidinyl)-N-methylbenzenesulfonamide;
3-({6-[(4-chlorophenyl)amino]-4-pyrimidinyl}amino)-N-methyl-4-(2-methyl-1-pyrrolidinyl)benzenesulfonamide;
3-({6-[(4-chlorophenyl)amino]-4-pyrimidinyl}amino)-N,4-dimethylbenzenesulfonamide;
3-(6-(4-chlorophenylamino)pyrimidin-4-ylamino)-4-(isobutylthio)-N-methylbenzenesulfonamide;
4-(isobutylthio)-N-methyl-3-(6-(4-(trifluoromethyl)phenylamino)pyrimidin-4-ylamino)benzenesulfonamide;
4-(isobutylthio)-3-(6-(4-isopropylphenylamino)pyrimidin-4-ylamino)-N-methylbenzenesulfonamide;
3-{[6-({4-[(difluoromethyl)oxy]phenyl}amino)-4-pyrimidinyl]amino}-N-methyl-4-[(2,2,2-trifluoroethyl)oxy]benzenesulfonamide;
N-methyl-4-[(2,2,2-trifluoroethyl)oxy]-3-{[6-({4-[(trifluoromethyl)oxy]phenyl}amino)-4-pyrimidinyl]amino}benzenesulfonamide;
3-({6-[(3,4-difluorophenyl)amino]-4-pyrimidinyl}amino)-N-methyl-4-[(2,2,2-trifluoroethyl)oxy]benzenesulfonamide;
3-({6-[(4-cyanophenyl)amino]-4-pyrimidinyl}amino)-N-methyl-4-[(2,2,2-trifluoroethyl)oxy]benzenesulfonamide;
3-(6-(4-chlorophenylamino)pyrimidin-4-ylamino)-4-(ethylthio)-N-methylbenzenesulfonamide;
4-(ethylthio)-N-methyl-3-(6-(4-(trifluoromethyl)phenylamino)pyrimidin-4-ylamino)benzenesulfonamide;
4-(ethylthio)-3-(6-(4-isopropylphenylamino)pyrimidin-4-ylamino)-N-methylbenzenesulfonamide;
3-(6-(4-chlorophenylamino)pyrimidin-4-ylamino)-N-methyl-4-(2,2,2-trifluoroethylthio)benzenesulfonamide;
N-methyl-4-(2,2,2-trifluoroethylthio)-3-(6-(4-(trifluoromethyl)phenylamino)pyrimidin-4-ylamino)benzenesulfonamide;
3-(6-(4-isopropylphenylamino)pyrimidin-4-ylamino)-N-methyl-4-(2,2,2-trifluoroethylthio)benzenesulfonamide;
4-fluoro-N-methyl-3-{[6-({4-[(trifluoromethyl)oxy]phenyl}amino)-4-pyrimidinyl]amino}benzenesulfonamide;
3-{[6-({4-[(difluoromethyl)oxy]phenyl}amino)-4-pyrimidinyl]amino}-4-fluoro-N-methylbenzenesulfonamide;
4-chloro-N-methyl-3-[(6-{[4-(trifluoromethyl)phenyl]amino}-4-pyrimidinyl)amino]benzenesulfonamide;
3-({6-[(4-cyanophenyl)amino]-4-pyrimidinyl}amino)-N-methyl-4-(methylsulfonyl)benzenesulfonamide;
3-({6-[(3,4-difluorophenyl)amino]-4-pyrimidinyl}amino)-N-methyl-4-(methylsulfonyl)benzenesulfonamide;
3-(6-(1H-indazol-5-ylamino)pyrimidin-4-ylamino)-N-methyl-4-(methylsulfonyl)benzenesulfonamide;
3-(6-(4-(cyanomethyl)phenylamino)pyrimidin-4-ylamino)-N-methyl-4-(methylsulfonyl)benzenesulfonamide;
4-(tert-butylsulfonyl)-3-(6-(4-chlorophenylamino)pyrimidin-4-ylamino)-N-methylbenzenesulfonamide;
3-({6-[(4-chlorophenyl)amino]-4-pyrimidinyl}amino)-N-methyl-4-[(2,2,2-trifluoro-1,1-dimethylethyl)oxy]benzenesulfonamide;
3-({6-[(3-bromophenyl)amino]-4-pyrimidinyl}amino)-N-methylbenzenesulfonamide;
3-({6-[(3-bromo-4-chlorophenyl)amino]-4-pyrimidinyl}amino)-N-methylbenzenesulfonamide;
3-[6-(3,4-dimethoxy-phenylamino)-pyrimidin-4-ylamino]-N-methyl-4-methylsulfanyl-benzenesulfonamide;
N-methyl-4-methylsulfanyl-3-[6-(3,4,5-trimethoxy-phenylamino)-pyrimidin-4-ylamino]-benzenesulfonamide;
3-[6-(3,5-dimethoxy-phenylamino)-pyrimidin-4-ylamino]-N-methyl-4-methylsulfanyl-benzenesulfonamide;
3-[6-(4-cyano-phenylamino)-pyrimidin-4-ylamino]-N-methyl-4-methylsulfanyl-benzenesulfonamide;
3-[6-(benzo[1,3]dioxol-5-ylamino)-pyrimidin-4-ylamino]-N-methyl-4-methylsulfanyl-benzenesulfonamide;
3-[6-(benzothiazol-6-ylamino)-pyrimidin-4-ylamino]-N-methyl-4-methylsulfanyl-benzenesulfonamide;
N-methyl-3-[6-(2-methyl-benzothiazol-5-ylamino)-pyrimidin-4-ylamino]-4-methylsulfanyl-benzenesulfonamide;
3-[6-(3-chloro-4-hydroxy-phenylamino)-pyrimidin-4-ylamino]-N-methyl-4-methylsulfanyl-benzenesulfonamide;
3-[6-(3,4-difluoro-phenylamino)-pyrimidin-4-ylamino]-N-methyl-4-methylsulfanyl-benzenesulfonamide;
N-methyl-4-methylsulfanyl-3-[6-(4-morpholin-4-yl-phenylamino)-pyrimidin-4-ylamino]-benzenesulfonamide;
3-[6-(2,3-dihydro-benzo[1,4]dioxin-6-ylamino)-pyrimidin-4-ylamino]-N-methyl-4-methylsulfanyl-benzenesulfonamide;
N-methyl-4-methylsulfanyl-3-[6-(4-piperidin-1-yl-phenylamino)-pyrimidin-4-ylamino]-benzenesulfonamide;
3-[6-(3-ethynyl-phenylamino)-pyrimidin-4-ylamino]-N-methyl-4-methylsulfanyl-benzenesulfonamide;
3-[6-(3,5-dichloro-4-hydroxy-phenylamino)-pyrimidin-4-ylamino]-N-methyl-4-methylsulfanyl-benzenesulfonamide;
N-methyl-4-methylsulfanyl-3-{6-[3-(2-methyl-thiazol-4-yl)-phenylamino]-pyrimidin-4-ylamino}-benzenesulfonamide;
3-(6-(3-methoxy-5-(trifluoromethyl)phenylamino)pyrimidin-4-ylamino)-N-methyl-4-(methylthio)benzenesulfonamide;
3-[6-(1H-indol-5-ylamino)-pyrimidin-4-ylamino]-N-methyl-4-methylsulfanyl-benzenesulfonamide;
N-methyl-4-methylsulfanyl-3-[6-(quinolin-6-ylamino)-pyrimidin-4-ylamino]-benzenesulfonamide;
3-[6-(3-chloro-4-cyano-phenylamino)-pyrimidin-4-ylamino]-N-methyl-4-methylsulfanyl-benzenesulfonamide;
N-methyl-4-methylsulfanyl-3-[6-(4-[1,2,4]-triazol-4-ylmethyl-phenylamino)-pyrimidin-4-ylamino]-benzenesulfonamide;
3-[6-(1H-indazol-5-ylamino)-pyrimidin-4-ylamino]-N-methyl-4-methylsulfanyl-benzenesulfonamide;
3-[6-(1H-indol-6-ylamino)-pyrimidin-4-ylamino]-N-methyl-4-methylsulfanyl-benzenesulfonamide;
N-methyl-4-(methylthio)-3-(6-(4-(piperazin-1-yl)phenylamino)pyrimidin-4-ylamino)benzenesulfonamide;
N-methyl-3-(6-(4-methyl-2-oxo-1,2-dihydroquinolin-7-ylamino)pyrimidin-4-ylamino)-4-(methylthio)benzenesulfonamide;
3-(6-(1-acetylindolin-6-ylamino)pyrimidin-4-ylamino)-N-methyl-4-(methylthio)benzenesulfonamide;
N-methyl-3-[6-(2-methyl-4-oxo-4H-chromen-7-ylamino)-pyrimidin-4-ylamino]-4-methylsulfanyl-benzenesulfonamide;
3-[6-(4-cyanomethyl-phenylamino)-pyrimidin-4-ylamino]-N-methyl-4-methylsulfanyl-benzenesulfonamide;
N-methyl-4-methylsulfanyl-3-[6-(5-oxo-5,6,7,8-tetrahydro-naphthalen-2-ylamino)-pyrimidin-4-ylamino]-benzenesulfonamide;
N-methyl-4-methylsulfanyl-3-[6-(3,4,5-trifluoro-phenylamino)-pyrimidin-4-ylamino]-benzenesulfonamide;
N-methyl-3-[6-(4-methyl-2-oxo-2H-chromen-7-ylamino)-pyrimidin-4-ylamino]-4-methylsulfanyl-benzenesulfonamide;
3-[6-(indan-5-ylamino)-pyrimidin-4-ylamino]-N-methyl-4-methylsulfanyl-benzenesulfonamide;
3-[6-(1H-indazol-6-ylamino)-pyrimidin-4-ylamino]-N-methyl-4-methylsulfanyl-benzenesulfonamide;
N-methyl-3-(6-(2-methyl-1,3-dioxoisoindolin-5-ylamino)pyrimidin-4-ylamino)-4-(methylthio)benzenesulfonamide;
3-[6-(3,5-dimethoxy-phenylamino)-pyrimidin-4-ylamino]-N-methyl-benzenesulfonamide;
N-methyl-3-[6-(3,4,5-trimethoxy-phenylamino)-pyrimidin-4-ylamino]-benzenesulfonamide;
3-[6-(3-ethynyl-phenylamino)-pyrimidin-4-ylamino]-N-methyl-benzenesulfonamide;
3-[6-(benzo[1,3]dioxol-5-ylamino)-pyrimidin-4-ylamino]-N-methyl-benzenesulfonamide;
3-[6-(3-chloro-4-hydroxy-phenylamino)-pyrimidin-4-ylamino]-N-methyl-benzenesulfonamide;
3-[6-(3,4-difluoro-phenylamino)-pyrimidin-4-ylamino]-N-methyl-benzenesulfonamide;
N-methyl-3-[6-(4-piperidin-1-yl-phenylamino)-pyrimidin-4-ylamino]-benzenesulfonamide;
3-[6-(4-cyano-phenylamino)-pyrimidin-4-ylamino]-N-methyl-benzenesulfonamide;
N-methyl-3-[6-(2-methyl-4-oxo-4H-chromen-7-ylamino)-pyrimidin-4-ylamino]-benzenesulfonamide;
3-[6-(3,5-dichloro-4-hydroxy-phenylamino)-pyrimidin-4-ylamino]-N-methyl-benzenesulfonamide;
N-methyl-3-{6-[3-(2-methyl-thiazol-4-yl)-phenylamino]-pyrimidin-4-ylamino}-benzenesulfonamide;
3-[6-(1H-indazol-5-ylamino)-pyrimidin-4-ylamino]-N-methyl-benzenesulfonamide;
N-methyl-3-[6-(5-oxo-5,6,7,8-tetrahydro-naphthalen-2-ylamino)-pyrimidin-4-ylamino]-benzenesulfonamide;
3-[6-(4-cyanomethyl-phenylamino)-pyrimidin-4-ylamino]-N-methyl-benzenesulfonamide;
N-methyl-3-[6-(4-methyl-2-oxo-2H-chromen-7-ylamino)-pyrimidin-4-ylamino]-benzenesulfonamide;
3-[6-(1-acetyl-2,3-dihydro-1H-indol-6-ylamino)-pyrimidin-4-ylamino]-N-methyl-benzenesulfonamide;
3-[6-(3-methoxy-5-trifluoromethyl-phenylamino)-pyrimidin-4-ylamino]-N-methyl-benzenesulfonamide;
N-methyl-3-[6-(4-methyl-2-oxo-1,2-dihydro-quinolin-7-ylamino)-pyrimidin-4-ylamino]-benzenesulfonamide;
N-methyl-3-[6-(3,4,5-trifluoro-phenylamino)-pyrimidin-4-ylamino]-benzenesulfonamide;
3-[6-(indan-5-ylamino)-pyrimidin-4-ylamino]-N-methyl-benzenesulfonamide
3-[6-(4-chloro-phenylamino)-pyrimidin-4-ylamino]-N-methyl-4-(propane-2-sulfonyl)-benzenesulfonamide;
3-(6-(3-bromo-5-methylphenylamino)pyrimidin-4-ylamino)-N-methyl-4-(methylsulfonyl)benzenesulfonamide;
3-(6-(1H-indol-6-ylamino)pyrimidin-4-ylamino)-N-methyl-4-(methylsulfonyl)benzenesulfonamide;
3-(6-(3-ethynylphenylamino)pyrimidin-4-ylamino)-N-methyl-4-(methylsulfonyl)benzenesulfonamide;
3-[6-(indan-5-ylamino)-pyrimidin-4-ylamino]-4-methanesulfonyl-N-methyl-benzenesulfonamide;
3-[6-(benzothiazol-6-ylamino)-pyrimidin-4-ylamino]-4-methanesulfonyl-N-methyl-benzenesulfonamide;
4-methanesulfonyl-N-methyl-3-[6-(5-oxo-5,6,7,8-tetrahydro-naphthalen-2-ylamino)-pyrimidin-4-ylamino]-benzenesulfonamide;
N-methyl-3-(6-(2-methylbenzo[d]thiazol-5-ylamino)pyrimidin-4-ylamino)-4-(methylsulfonyl)benzenesulfonamide;
N-methyl-4-(methylsulfonyl)-3-[(6-{[4-(1H-1,2,4-triazol-1-ylmethyl)phenyl]amino}-4-pyrimidinyl)amino]benzenesulfonamide;
3-[6-(1H-indol-5-ylamino)-pyrimidin-4-ylamino]-4-methanesulfonyl-N-methyl-benzenesulfonamide;
4-methanesulfonyl-N-methyl-3-[6-(2-methyl-4-oxo-4H-chromen-7-ylamino)-pyrimidin-4-ylamino]-benzenesulfonamide;
5-({6-[(4-chlorophenyl)amino]-4-pyrimidinyl}amino)-2-fluoro-N-methylbenzenesulfonamide;
5-(6-(4-chlorophenylamino)pyrimidin-4-ylamino)-2-fluoro-N-methyl-4-(1,1,1-trifluoropropan-2-yloxy)benzenesulfonamide;
1-{6-[(4-chlorophenyl)amino]-4-pyrimidinyl}-N-methyl-2,3-dihydro-1H-indole-6-sulfonamide;
3-[(6-{[3,4-bis(methyloxy)phenyl]amino]-4-pyrimidinyl}amino)-N-methylbenzenesulfonamide;
3-({6-[(3,4-dichlorophenyl)amino]-4-pyrimidinyl}amino)-N-methylbenzenesulfonamide;
3-({6-[(3,4-dimethylphenyl)amino]-4-pyrimidinyl}amino)-N-methylbenzenesulfonamide;
N-methyl-3-[(6-{[3-(1-methylethyl)phenyl]amino}-4-pyrimidinyl)amino]benzenesulfonamide;
3-[(6-{[3-(1,1-dimethylethyl)phenyl]amino}-4-pyrimidinyl)amino]-N-methylbenzenesulfonamide;
3-[(6-{[3-(ethyloxy)phenyl]amino}-4-pyrimidinyl)amino]-N-methylbenzenesulfonamide;
3-({6-[(4-fluorophenyl)amino]-4-pyrimidinyl}amino)-N-methylbenzenesulfonamide;
N-methyl-3-[(6-{[3-(1-pyrrolidinyl)phenyl]amino}-4-pyrimidinyl)amino]benzenesulfonamide;
N-methyl-3-[(6-{[3-(4-methyl-1-piperazinyl)phenyl]amino}-4-pyrimidinyl)amino]benzenesulfonamide;
3-({6-[(3,5-dichlorophenyl)amino]-4-pyrimidinyl}amino)-N-methylbenzenesulfonamide;
N-methyl-3-({6-[(2-oxo-2,3-dihydro-1H-indol-5-yl)amino]-4-pyrimidinyl}amino)benzenesulfonamide;
N-methyl-3-({6-[(2-oxo-2,3-dihydro-1,3-benzoxazol-6-yl)amino]-4-pyrimidinyl}amino)benzenesulfonamide;
N-methyl-3-({6-[(2-oxo-2,3-dihydro-1H-benzimidazol-5-yl)amino]-4-pyrimidinyl}amino)benzenesulfonamide;
N-methyl-3-({6-[(2-oxo-1,2,3,4-tetrahydro-7-quinolinyl)amino]-4-pyrimidinyl}amino)benzenesulfonamide;
3-({6-[(3-bromo-5-chlorophenyl)amino]-4-pyrimidinyl}amino)-N-methylbenzenesulfonamide;
3-({6-[(3,5-dimethylphenyl)amino]-4-pyrimidinyl}amino)-N-methylbenzenesulfonamide;
N-methyl-3-{[6-({4-[(methylamino)sulfonyl]phenyl}amino)-4-pyrimidinyl]amino}benzenesulfonamide;
N-methyl-3-[(6-{[3-(1-pyrrolidinylmethyl)phenyl]amino}-4-pyrimidinyl)amino]benzenesulfonamide;
N-methyl-3-({6-[(4-{[2-(4-morpholinyl)ethyl]oxy}phenyl)amino]-4-pyrimidinyl}amino)benzenesulfonamide;
3-({6-[(4-{[2-(dimethylamino)ethyl]oxy}phenyl)amino]-4-pyrimidinyl}amino)-N-methylbenzenesulfonamide;
N-methyl-3-{[6-({3-[(4-methyl-1-piperazinyl)methyl]phenyl}amino)-4-pyrimidinyl]amino}benzenesulfonamide;
N-methyl-3-[(6-{[4-(trifluoromethyl)phenyl]amino}-4-pyrimidinyl)amino]benzenesulfonamide;
N-methyl-3-[(6-{[4-(1-methylethyl)phenyl]amino}-4-pyrimidinyl)amino]benzenesulfonamide;
N-methyl-3-{[6-({4-[(1-methylethyl)oxy]phenyl}amino)-4-pyrimidinyl]amino}benzenesulfonamide;
3-{[6-({4-[(difluoromethyl)oxy]phenyl}amino)-4-pyrimidinyl]amino}-N-methylbenzenesulfonamide;
N-methyl-3-[(6-{[4-(2-oxo-1-pyrrolidinyl)phenyl]amino}-4-pyrimidinyl)amino]benzenesulfonamide;
3-[(6-{[3-chloro-4-(methyloxy)phenyl]amino}-4-pyrimidinyl)amino]-N-methylbenzenesulfonamide;
3-({6-[(4-cyclopropylphenyl)amino]-4-pyrimidinyl}amino)-N-methylbenzenesulfonamide;
N-methyl-3-[(6-{[4-(1H-pyrazol-1-yl)phenyl]amino}-4-pyrimidinyl)amino]benzenesulfonamide;
3-[(6-{[4-(3,5-dimethyl-1H-pyrazol-1-yl)phenyl]amino}-4-pyrimidinyl)amino]-N-methylbenzenesulfonamide;
3-[(6-{[4-chloro-3-(methyloxy)phenyl]amino}-4-pyrimidinyl)amino]-N-methylbenzenesulfonamide;
N-methyl-3-[(6-{[4-(2-thienyl)phenyl]amino}-4-pyrimidinyl)amino]benzenesulfonamide;
N-methyl-3-[(6-{[4-(2-methyl-1H-imidazol-1-yl)phenyl]amino}-4-pyrimidinyl)amino]benzenesulfonamide;
N-methyl-3-[(6-{[4-(1-methylpropyl)phenyl]amino}-4-pyrimidinyl)amino]benzenesulfonamide;
N-methyl-3-{[6-(6-quinolinylamino)-4-pyrimidinyl]amino}benzenesulfonamide;
N-methyl-3-{[6-({4-[(trifluoromethyl)thio]phenyl}amino)-4-pyrimidinyl]amino}benzenesulfonamide;
3-({6-[(4-bromophenyl)amino]-4-pyrimidinyl}amino)-N-methylbenzenesulfonamide;
N-methyl-3-[(6-{[4-(methylthio)phenyl]amino}-4-pyrimidinyl)amino]benzenesulfonamide;
N-methyl-3-{[6-({4-[(trifluoromethyl)oxy]phenyl}amino)-4-pyrimidinyl]amino}benzenesulfonamide;
3-({6-[(4-chlorophenyl)amino]-4-pyrimidinyl}amino)-4-(dimethylamino)-N-methylbenzenesulfonamide;
4-(dimethylamino)-N-methyl-3-({6-[(3-methylphenyl)amino]-4-pyrimidinyl}amino)benzenesulfonamide;
N-methyl-1-(6-{[4-(trifluoromethyl)phenyl]amino}-4-pyrimidinyl)-2,3-dihydro-1H-indole-6-sulfonamide;
1-{6-[(4-chlorophenyl)amino]-4-pyrimidinyl}-N-methyl-1H-benzimidazole-6-sulfonamide;
3-({6-[(5-bromo-6-methyl-2-pyridinyl)amino]-4-pyrimidinyl}amino)-N-methyl-4-[(2,2,2-trifluoroethyl)oxy]benzenesulfonamide;
3-({6-[(4-chlorophenyl)amino]-4-pyrimidinyl}amino)-N-methyl-4-[(1-methylethyl)oxy]benzenesulfonamide;
3-({6-[(4-chlorophenyl)amino]-4-pyrimidinyl}amino)-N-methyl-4-(4-morpholinyl)benzenesulfonamide;
3-({6-[(4-chlorophenyl)amino]-4-pyrimidinyl}amino)-N-methyl-4-(methyloxy)benzenesulfonamide;
3-({6-[(4-chlorophenyl)amino]-4-pyrimidinyl}amino)-4-[ethyl(methyl)amino]-N-methylbenzenesulfonamide;
3-({6-[(4-chlorophenyl)amino]-4-pyrimidinyl}amino)-4-hydroxy-N-methylbenzenesulfonamide;
3-({6-[(4-chlorophenyl)amino]-4-pyrimidinyl}amino)-4-fluoro-N-methylbenzenesulfonamide;
3-({6-[(4-chlorophenyl)amino]-4-pyrimidinyl}amino)-N-methyl-4-(methylthio)benzenesulfonamide;
3-({6-[(4-chlorophenyl)amino]-4-pyrimidinyl}amino)-N-methyl-4-[(trifluoromethyl)oxy]benzenesulfonamide;
3-({6-[(4-chlorophenyl)amino]-4-pyrimidinyl}amino)-N-methyl-4-[(2R)-2-(trifluoromethyl)-1-pyrrolidinyl]benzenesulfonamide;
3-({6-[(4-chlorophenyl)amino]-4-pyrimidinyl}amino)-4-(3,3-difluoro-1-pyrrolidinyl)-N-methylbenzenesulfonamide;
N-methyl-3-[(6-{[4-(1,3-oxazol-5-yl)phenyl]amino}-4-pyrimidinyl)amino]benzenesulfonamide;
N-methyl-3-({6-[(3-methylphenyl)amino]-4-pyrimidinyl}amino)-4-(4-morpholinyl)benzenesulfonamide;
N-methyl-4-(methyloxy)-3-[(6-{[4-(trifluoromethyl)phenyl]amino}-4-pyrimidinyl)amino]benzenesulfonamide;
N-methyl-4-(methylthio)-3-[(6-{[4-(trifluoromethyl)phenyl]amino}-4-pyrimidinyl)amino]benzenesulfonamide;
3-({6-[(3-bromo-5-methylphenyl)amino]-4-pyrimidinyl}amino)-N-methyl-4-(methyloxy)benzenesulfonamide;
1-{6-[(3-bromo-5-methylphenyl)amino]-4-pyrimidinyl}-N-methyl-2,3-dihydro-1H-indole-6-sulfonamide;
N-methyl-3-{[6-({4-[(2,2,2-trifluoroethyl)oxy]phenyl}amino)-4-pyrimidinyl]amino}-4-[(2,2,2-trifluoroethyl)thio]benzenesulfonamide;
3-({6-[(3,4-difluorophenyl)amino]-4-pyrimidinyl}amino)-N-methyl-4-[(trifluoromethyl)oxy]benzenesulfonamide;
N-methyl-3-{[6-(4-pyridinylamino)-4-pyrimidinyl]amino}benzenesulfonamide;
N-methyl-3-{[6-(3-pyridinylamino)-4-pyrimidinyl]amino}benzenesulfonamide;
N-methyl-3-({6-[(5-methyl-3-pyridinyl)amino]-4-pyrimidinyl}amino)benzenesulfonamide;
N-methyl-3-{[6-(2-pyridinylamino)-4-pyrimidinyl]amino}benzenesulfonamide;
N-methyl-5-{[6-({3-[(methylamino)sulfonyl]phenyl}amino)-4-pyrimidinyl]amino}-3-pyridinesulfonamide;
3-({6-[(5-chloro-2-pyridinyl)amino]-4-pyrimidinyl}amino)-N-methylbenzenesulfonamide;
N-methyl-3-{[6-(1,3-thiazol-2-ylamino)-4-pyrimidinyl]amino}benzenesulfonamide;
N-methyl-3-[(6-{[5-(trifluoromethyl)-2-pyridinyl]amino}-4-pyrimidinyl)amino]benzenesulfonamide;
N-methyl-3-({6-[(5-methyl-1,3-thiazol-2-yl)amino]-4-pyrimidinyl}amino)benzenesulfonamide;
N-methyl-3-{[6-(1,3,4-thiadiazol-2-ylamino)-4-pyrimidinyl]amino}benzenesulfonamide;
3-{[6-(3-isoquinolinylamino)-4-pyrimidinyl]amino}-N-methylbenzenesulfonamide;
N-methyl-3-{[6-(2-quinolinylamino)-4-pyrimidinyl]amino}benzenesulfonamide;
N-methyl-3-{[6-(1,3-oxazol-2-ylamino)-4-pyrimidinyl]amino}benzenesulfonamide;
N-methyl-3-[(6-{[4-(trifluoromethyl)-1,3-thiazol-2-yl]amino}-4-pyrimidinyl)amino]benzenesulfonamide;
methyl (2-{[6-({3-[(methylamino)sulfonyl]phenyl}amino)-4-pyrimidinyl]amino}-1,3-thiazol-4-yl)acetate;
N-methyl-3-[(6-{[4-(1-methylethyl)-1,3-thiazol-2-yl]amino}-4-pyrimidinyl)amino]benzenesulfonamide;
N-methyl-3-({6-[(4-methyl-1,3-oxazol-2-yl)amino]-4-pyrimidinyl}amino)benzenesulfonamide;
N-methyl-4-(methyloxy)-3-{[6-(2-pyridinylamino)-4-pyrimidinyl]amino}benzenesulfonamide;
3-({6-[(5-chloro-2-pyridinyl)amino]-4-pyrimidinyl}amino)-N-methyl-4-(methyloxy)benzenesulfonamide;
3-({6-[(5-chloro-2-pyridinyl)amino]-4-pyrimidinyl}amino)-N-methyl-4-[(2,2,2-trifluoroethyl)oxy]benzenesulfonamide;
N-methyl-3-{[6-(2-pyridinylamino)-4-pyrimidinyl]amino}-4-[(2,2,2-trifluoroethyl)oxy]benzenesulfonamide;
3-({6-[(5-chloro-2-pyridinyl)amino]-4-pyrimidinyl}amino)-N-methyl-4-(methylthio)benzenesulfonamide;
1-{6-[(5-chloro-2-pyridinyl)amino]-4-pyrimidinyl}-N-methyl-2,3-dihydro-1H-indole-6-sulfonamide;
N-methyl-4-[(2,2,2-trifluoroethyl)oxy]-3-[(6-{[5-(trifluoromethyl)-2-pyridinyl]amino}-4-pyrimidinyl)amino]benzenesulfonamide;
N-methyl-3-{[6-(4-pyridinylamino)-4-pyrimidinyl]amino}-4-[(2,2,2-trifluoroethyl)oxy]benzenesulfonamide;
3-({6-[(3-fluoro-2-pyridinyl)amino]-4-pyrimidinyl}amino)-N-methyl-4-[(2,2,2-trifluoroethyl)oxy]benzenesulfonamide;
3-({6-[(5-cyano-2-pyridinyl)amino]-4-pyrimidinyl}amino)-N-methyl-4-[(2,2,2-trifluoroethyl)oxy]benzenesulfonamide;
N-methyl-3-{[6-(4-pyrimidinylamino)-4-pyrimidinyl]amino}-4-[(2,2,2-trifluoroethyl)oxy]benzenesulfonamide;
3-({6-[(5-chloro-3-fluoro-2-pyridinyl)amino]-4-pyrimidinyl}amino)-N-methyl-4-[(2,2,2-trifluoroethyl)oxy]benzenesulfonamide;
N-methyl-4-[(2,2,2-trifluoroethyl)oxy]-3-[(6-{[6-(trifluoromethyl)-3-pyridinyl]amino}-4-pyrimidinyl)amino]benzenesulfonamide;
3-({6-[(5-chloro-4-methyl-2-pyridinyl)amino]-4-pyrimidinyl}amino)-N-methyl-4-[(2,2,2-trifluoroethyl)oxy]benzenesulfonamide;
3-({6-[(4,5-dichloro-2-pyridinyl)amino]-4-pyrimidinyl}amino)-N-methyl-4-[(2,2,2-trifluoroethyl)oxy]benzenesulfonamide;
3-({6-[(5-chloro-6-methyl-2-pyridinyl)amino]-4-pyrimidinyl}amino)-N-methyl-4-[(2,2,2-trifluoroethyl)oxy]benzenesulfonamide;
3-(6-(5-isopropylpyridin-2-ylamino)pyrimidin-4-ylamino)-N-methyl-4-(2,2,2-trifluoroethoxy)benzenesulfonamide;
3-({6-[(5-chloro-2-pyridinyl)amino]-4-pyrimidinyl}amino)-4-fluoro-N-methylbenzenesulfonamide;
4-fluoro-N-methyl-3-[(6-{[5-(trifluoromethyl)-2-pyridinyl]amino}-4-pyrimidinyl)amino]benzenesulfonamide;
4-chloro-3-({6-[(5-chloro-2-pyridinyl)amino]-4-pyrimidinyl}amino)-N-methylbenzenesulfonamide;
3-({6-[(5-chloro-2-pyridinyl)amino]-4-pyrimidinyl}amino)-N-methyl-4-(methylsulfonyl)benzenesulfonamide;
N-methyl-4-(methylsulfonyl)-3-[(6-{[5-(trifluoromethyl)-2-pyridinyl]amino}-4-pyrimidinyl)amino]benzenesulfonamide;
N-methyl-4-(methylsulfonyl)-3-{[6-(6-quinolinylamino)-4-pyrimidinyl]amino}benzenesulfonamide;
3-({6-[(5-chloro-2-pyridinyl)amino]-4-pyrimidinyl}amino)-N-methyl-4-[(2,2,2-trifluoro-1-methylethyl)oxy]benzenesulfonamide;
N-methyl-4-[(2,2,2-trifluoro-1-methylethyl)oxy]-3-[(6-{[5-(trifluoromethyl)-2-pyridinyl]amino}-4-pyrimidinyl)amino]benzenesulfonamide;
4-(tert-butylsulfonyl)-N-methyl-3-(6-(5-(trifluoromethyl)pyridin-2-ylamino)pyrimidin-4-ylamino)benzenesulfonamide;
4-(tert-butylsulfonyl)-3-(6-(5-chloropyridin-2-ylamino)pyrimidin-4-ylamino)-N-methylbenzenesulfonamide;
N-methyl-4-(propane-2-sulfonyl)-3-[6-(5-trifluoromethyl-pyridin-2-ylamino)-pyrimidin-4-ylamino]-benzenesulfonamide;
3-[6-(5-chloro-pyridin-2-ylamino)-pyrimidin-4-ylamino]-N-methyl-4-(propane-2-sulfonyl)-benzenesulfonamide;
3-({6-[(5-chloro-2-pyridinyl)amino]-4-pyrimidinyl}amino)-N-methyl-4-[(trifluoromethyl)oxy]benzenesulfonamide;
1-[6-(5-chloro-pyridin-2-ylamino)-pyrimidin-4-yl]-3,3-dimethyl-2,3-dihydro-1H-indole-6-sulfonic acid methylamide;
5-(6-(5-chloropyridin-2-ylamino)pyrimidin-4-ylamino)-2-fluoro-N-methyl-4-(1,1,1-trifluoropropan-2-yloxy)benzenesulfonamide;
5-[6-(5-chloro-pyridin-2-ylamino)-pyrimidin-4-ylamino]-2-fluoro-4-methanesulfonyl-N-methyl-benzenesulfonamide;
5-({6-[(5-chloro-2-pyridinyl)amino]-4-pyrimidinyl}amino)-2-fluoro-N-methyl-4-[(2,2,2-trifluoroethyl)oxy]benzenesulfonamide;
2-fluoro-N-methyl-4-[(2,2,2-trifluoroethyl)oxy]-5-[(6-{[5-(trifluoromethyl)-2-pyridinyl]amino}-4-pyrimidinyl)amino]benzenesulfonamide;
3-({6-[(5-fluoro-2-pyridinyl)amino]-4-pyrimidinyl}amino)-N-methyl-4-[(2,2,2-trifluoroethyl)oxy]benzenesulfonamide;
3-({6-[(5-chloro-2-pyridinyl)amino]-4-pyrimidinyl}amino)-4-(ethylsulfonyl)-N-methylbenzenesulfonamide;
4-(ethylsulfonyl)-N-methyl-3-[(6-{[5-(trifluoromethyl)-2-pyridinyl]amino}-4-pyrimidinyl)amino]benzenesulfonamide;
3-({6-[(5-cyano-2-pyridinyl)amino]-4-pyrimidinyl}amino)-N-methyl-4-(methylsulfonyl)benzenesulfonamide;
3-({6-[(5-cyano-2-pyridinyl)amino]-4-pyrimidinyl}amino)-N-methyl-4-[(2,2,2-trifluoro-1-methylethyl)oxy]benzenesulfonamide;
2-{[6-({3-[(methylamino)sulfonyl]phenyl}amino)-4-pyrimidinyl]amino}-1,3-thiazole-5-carboxylic acid;
(2-{[6-({3-[(methylamino)sulfonyl]phenyl}amino)-4-pyrimidinyl]amino}-1,3-thiazol-4-yl)acetic acid;
1-{6-[(4-chlorophenyl)amino]-4-pyrimidinyl}-N-methyl-1H-indole-6-sulfonamide;
3-{6-[(4-chlorophenyl)amino]-4-pyrimidinyl}-N-methyl-2-oxo-2,3-dihydro-1H-benzimidazole-5-sulfonamide;
3-{[6-({3-[6-(dimethylamino)-3-pyridinyl]phenyl}amino)-4-pyrimidinyl]amino}-N-methylbenzenesulfonamide;
N-methyl-3-({6-[(5-methyl-3-biphenylyl)amino]-4-pyrimidinyl}amino)benzenesulfonamide;
N-methyl-3-[(6-{[3-methyl-5-(3-pyridinyl)phenyl]amino}-4-pyrimidinyl)amino]benzenesulfonamide;
3-[(6-{[3′-(dimethylamino)-3-biphenylyl]amino}-4-pyrimidinyl)amino]-N-methylbenzenesulfonamide;
N-methyl-3-[(6-{[4′-(4-morpholinyl)-3-biphenyl]amino}-4-pyrimidinyl)amino]-benzenesulfonamide;
N-methyl-3-{[6-({3-[6-(methyloxy)-3-pyridinyl]phenyl}amino)-4-pyrimidinyl]amino}-benzenesulfonamide;
3′-{[6-({3-[(methylamino)sulfonyl]phenyl}amino)-4-pyrimidinyl]amino}-4-biphenylcarboxamide;
N-methyl-3-{[6-({3-[5-(methyloxy)-3-pyridinyl]phenyl}amino)-4-pyrimidinyl]amino}-benzenesulfonamide;
3′-{[6-({3-[(methylamino)sulfonyl]phenyl}amino)-4-pyrimidinyl]amino}-3-biphenylcarboxamide;
N-methyl-3-{[6-({3′-[(methylsulfonyl)amino]-3-biphenylyl]amino)-4-pyrimidinyl}amino}benzenesulfonamide;
3-[(6-{[4′-(dimethylamino)-3-biphenylyl]amino}-4-pyrimidinyl)amino]-N-methylbenzenesulfonamide;
N-methyl-3-{[6-({3-[4-(methyloxy)-3-pyridinyl]phenyl}amino)-4-pyrimidinyl]amino}-benzenesulfonamide;
N-(3′-{[6-({3-[(methylamino)sulfonyl]phenyl}amino)-4-pyrimidinyl]amino}-4-biphenylyl)acetamide;
N-methyl-3-{[6-({4′-[(methylsulfonyl)amino]-3-biphenylyl]amino)-4-pyrimidinyl}amino}-benzenesulfonamide;
N-(3′-{[6-({3-[(methylamino)sulfonyl]phenyl}amino)-4-pyrimidinyl]amino}-3-biphenylyl)acetamide;
N-methyl-3′-{[6-({3-[(methylamino)sulfonyl]phenyl}amino)-4-pyrimidinyl]amino}-4-biphenylsulfonamide;
N-methyl-3′-{[6-({3-[(methylamino)sulfonyl]phenyl}amino)-4-pyrimidinyl]amino}-3-biphenylsulfonamide;
3-[(6-{[4-chloro-3-(3-pyridinyl)phenyl]amino}-4-pyrimidinyl)amino]-N-methylbenzenesulfonamide;
2′-chloro-5′-{[6-({3-[(methylamino)sulfonyl]phenyl}amino)-4-pyrimidinyl]amino}-3-biphenylcarboxamide;
3-[(6-{[6-chloro-3′-(4-morpholinyl)-3-biphenylyl]amino}-4-pyrimidinyl)amino]-N-methylbenzenesulfonamide;
4-{[6-({3-[(methylamino)sulfonyl]phenyl}amino)-4-pyrimidinyl]amino}benzoic acid;
[(3-{[6-({3-[(methylamino)sulfonyl]phenyl}amino)-4-pyrimidinyl]amino}phenyl)oxy]acetic acid;
N,N-dimethyl-4-{[6-({3-[(methylamino)sulfonyl]phenyl}amino)-4-pyrimidinyl]amino}benzamide;
N,N-dimethyl-2-[(3-{[6-({3-[(methylamino)sulfonyl]phenyl}amino)-4-pyrimidinyl]amino}phenyl)oxy]acetamide;
N-(2-hydroxyethyl)-4-{[6-({3-[(methylamino)sulfonyl]phenyl}amino)-4-pyrimidinyl]amino}benzamide;
N-methyl-3-{[6-({4-[(4-methyl-1-piperazinyl)carbonyl]phenyl}amino)-4-pyrimidinyl]amino}benzenesulfonamide;
4-{[6-({3-[(methylamino)sulfonyl]phenyl}amino)-4-pyrimidinyl]amino}-N-(1-methyl-4-piperidinyl)benzamide;
N-methyl-3-[(6-{[4-(1-piperazinylcarbonyl)phenyl]amino}-4-pyrimidinyl)amino]benzenesulfonamide;
N-methyl-3-[(6-{[4-({4-[2-(methyloxy)ethyl]-1-piperazinyl]carbonyl)phenyl]amino}-4-pyrimidinyl)amino}benzenesulfonamide;
4-{[6-({3-[(methylamino)sulfonyl]phenyl}amino)-4-pyrimidinyl]amino}-N-[2-(methyloxy)ethyl]benzamide;
4-{[6-({3-[(methylamino)sulfonyl]phenyl}amino)-4-pyrimidinyl]amino}-N-[3-(methyloxy)propyl]benzamide;
N-[2-(dimethylamino)ethyl]-4-{[6-({3-[(methylamino)sulfonyl]phenyl}amino)-4-pyrimidinyl]amino}benzamide;
N,N-diethyl-4-{[6-({3-[(methylamino)sulfonyl]phenyl}amino)-4-pyrimidinyl]amino}benzamide;
N-methyl-3-[(6-{[4-(1-pyrrolidinylcarbonyl)phenyl]amino}-pyrimidinyl)amino]benzenesulfonamide;
3-({6-[(4-{[(3S)-3-(dimethylamino)-1-pyrrolidinyl]carbonyl}phenyl)amino]-4-pyrimidinyl}amino)-N-methylbenzenesulfonamide;
N-methyl-3-{[6-({4-[(4-methylhexahydro-1H-1,4-diazepin-1-yl)carbonyl]phenyl}amino)-4-pyrimidinyl]amino}benzenesulfonamide;
N-methyl-3-[(6-{[4-(4-thiomorpholinylcarbonyl)phenyl]amino}-4-pyrimidinyl)amino]benzenesulfonamide;
3-{[6-({4-[(4,4-difluoro-1-piperidinyl)carbonyl]phenyl}amino)-4-pyrimidinyl]amino}-N-methylbenzenesulfonamide;
3-({6-[(4-{[(3R)-3-(dimethylamino)-1-pyrrolidinyl]carbonyl}phenyl)amino]-4-pyrimidinyl}amino)-N-methylbenzenesulfonamide;
N-[2-(dimethylamino)ethyl]-N-methyl-4-{[6-({3-[(methylamino)sulfonyl]phenyl}amino)-4-pyrimidinyl]amino}benzamide;
N-[2-(dimethylamino)ethyl]-N-methyl-4-[(6-{[5-[(methylamino)sulfonyl]-2-(methylthio)phenyl]amino}-4-pyrimidinyl)amino]benzamide;
N-[(4-{[6-({3-[(methylamino)sulfonyl]phenyl}amino)-4-pyrimidinyl]amino}phenyl)carbonyl]glycine;
N-methyl-3-[(6-{[3-(6-oxo-1,6-dihydro-3-pyridinyl)phenyl]amino}-4-pyrimidinyl)amino]benzenesulfonamide;
3-({6-[(3-hydroxyphenyl)amino]-4-pyrimidinyl}amino)-N-methylbenzenesulfonamide;
N-methyl-4-(methylsulfonyl)-3-[(6-{[4-(trifluoromethyl)phenyl]amino}-4-pyrimidinyl)amino]benzenesulfonamide;
3-({6-[(4-chlorophenyl)amino]-4-pyrimidinyl}amino)-N-methyl-4-(methylsulfonyl)benzenesulfonamide;
3-(6-(4-chlorophenylamino)pyrimidin-4-ylamino)-4-(isobutylsulfonyl)-N-methylbenzenesulfonamide;
3-(6-(4-chlorophenylamino)pyrimidin-4-ylamino)-4-(ethylsulfonyl)-N-methylbenzenesulfonamide;
3-({6-[(4-chlorophenyl)amino]-4-pyrimidinyl}amino)-N-methyl-4-[(2,2,2-trifluoro-1-methylethyl)oxy]benzenesulfonamide;
3-({6-[(4-chlorophenyl)amino]-4-pyrimidinyl}amino)-N-methyl-4-[(2,2,2-trifluoro-1-methylethyl)oxy]benzenesulfonamide;
3-({6-[(5-chloro-2-pyridinyl)amino]-4-pyrimidinyl}amino)-N-methyl-4-[(2,2,2-trifluoro-1-methylethyl)oxy]benzenesulfonamide; and
3-({6-[(5-chloro-2-pyridinyl)amino]-4-pyrimidinyl}amino)-N-methyl-4-[(2,2,2-trifluoro-1-methylethyl)oxy]benzenesulfonamide;

Representative compounds of this invention include the compounds of Examples 1-380.

The compounds according to Formula I may contain one or more asymmetric centers (also referred to as a chiral center) and may, therefore, exist as individual enantiomers, diastereomers, or other stereoisomeric forms, or as mixtures thereof. Chiral centers, such as chiral carbon atoms, may also be present in a substituent such as an alkyl group. Where the stereochemistry of a chiral center present in Formula I, or in any chemical structure illustrated herein, is not specified the structure is intended to encompass all individual stereoisomers and all mixtures thereof. Thus, compounds according to Formula I containing one or more chiral center may be used as racemic mixtures, enantiomerically enriched mixtures, or as enantiomerically pure individual stereoisomers.

Individual stereoisomers of a compound according to Formula I which contain one or more asymmetric centers may be resolved by methods known to those skilled in the art. For example, such resolution may be carried out (1) by formation of diastereoisomeric salts, complexes or other derivatives; (2) by selective reaction with a stereoisomer-specific reagent, for example by enzymatic oxidation or reduction; or (3) by gas-liquid or liquid chromatography in a chiral environment, for example, on a chiral support such as silica with a bound chiral ligand or in the presence of a chiral solvent. The skilled artisan will appreciate that where the desired stereoisomer is converted into another chemical entity by one of the separation procedures described above, a further step is required to liberate the desired form. Alternatively, specific stereoisomers may be synthesized by asymmetric synthesis using optically active reagents, substrates, catalysts or solvents, or by converting one enantiomer to the other by asymmetric transformation.

When a disclosed compound or its salt is named or depicted by structure, it is to be understood that the compound or salt, including solvates (particularly, hydrates) thereof, may exist in crystalline forms, non-crystalline forms or a mixture thereof. The compound or salt, or solvates (particularly, hydrates) thereof, may also exhibit polymorphism (i.e. the capacity to occur in different crystalline forms). These different crystalline forms are typically known as “polymorphs.” It is to be understood that when named or depicted by structure, the disclosed compound, or solvates (particularly, hydrates) thereof, also include all polymorphs thereof. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. One of ordinary skill in the art will appreciate that different polymorphs may be produced, for example, by changing or adjusting the conditions used in crystallizing/recrystallizing the compound.

For solvates of the compounds of the invention, or salts thereof, that are in crystalline form, the skilled artisan will appreciate that pharmaceutically-acceptable solvates may be formed wherein solvent molecules are incorporated into the crystalline lattice during crystallization. Solvates may involve nonaqueous solvents such as ethanol, isopropanol, DMSO, acetic acid, ethanolamine, and ethyl acetate, or they may involve water as the solvent that is incorporated into the crystalline lattice. Solvates wherein water is the solvent that is incorporated into the crystalline lattice are typically referred to as “hydrates.” Hydrates include stoichiometric hydrates as well as compositions containing variable amounts of water. The invention includes all such solvates.

Because of their potential use in medicine, the salts of the compounds of Formula I are preferably pharmaceutically acceptable. The compounds of this invention are bases, wherein a desired salt form may be prepared by any suitable method known in the art, including treatment of the free base with an inorganic acid, such as hydrochloric acid, hydrobromic acid, sulfuric acid, nitric acid, phosphoric acid, and the like, or with an organic acid, such as acetic acid, trifluoroacetic acid, maleic acid, succinic acid, mandelic acid, fumaric acid, malonic acid, pyruvic acid, oxalic acid, glycolic acid, salicylic acid, pyranosidyl acid, such as glucuronic acid or galacturonic acid, alpha-hydroxy acid, such as citric acid or tartaric acid, amino acid, such as aspartic acid or glutamic acid, aromatic acid, such as benzoic acid or cinnamic acid, sulfonic acid, such as p-toluenesulfonic acid, methanesulfonic acid, ethanesulfonic acid or the like. Examples of pharmaceutically acceptable salts include sulfates, pyrosulfates, bisulfates, sulfites, bisulfites, phosphates, chlorides, bromides, iodides, acetates, propionates, decanoates, caprylates, acrylates, formates, isobutyrates, caproates, heptanoates, propiolates, oxalates, malonates succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, phenylacetates, phenylpropionates, phenylbutrates, citrates, lactates, γ-hydroxybutyrates, glycolates, tartrates mandelates, and sulfonates, such as xylenesulfonates, methanesulfonates, propanesulfonates, naphthalene-1-sulfonates and naphthalene-2-sulfonates.

Salts of the disclosed compounds containing a carboxylic acid or other acidic functional group can be prepared by reacting with a suitable base. Such a pharmaceutically acceptable salt may be made with a base which affords a pharmaceutically acceptable cation, which includes alkali metal salts (especially sodium and potassium), alkaline earth metal salts (especially calcium and magnesium), aluminum salts and ammonium salts, as well as salts made from physiologically acceptable organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acid such as lysine and arginine.

If an inventive basic compound is isolated as a salt, the corresponding free base form of that compound may be prepared by any suitable method known to the art, including treatment of the salt with an inorganic or organic base, suitably an inorganic or organic base having a higher pKₐ than the free base form of the compound. Similarly, if a disclosed compound containing a carboxylic acid or other acidic functional group is isolated as a salt, the corresponding free acid form of that compound may be prepared by any suitable method known to the art, including treatment of the salt with an inorganic or organic acid, suitably an inorganic or organic acid having a lower pKₐ than the free acid form of the compound.

### General Methods of Preparation

The compounds of Formula I may be obtained by using synthetic procedures illustrated in the Schemes below or by drawing on the knowledge of a skilled organic chemist. The synthesis provided in these Schemes are applicable for producing compounds of the invention having a variety of different R¹, R², R³, R⁴, R⁵, R⁶, R⁷ and R⁸ groups employing appropriate precursors, which are suitably protected if needed, to achieve compatibility with the reactions outlined herein. Subsequent deprotection, where needed, affords compounds of the nature generally disclosed. While the Schemes are shown with compounds only of Formula I, they are illustrative of processes that may be used to make the compounds of the invention.

Compound names were generated using the software naming program ACD/Name Pro V6.02 available from Advanced Chemistry Development, Inc., 110 Yonge Street, 14^{th} Floor, Toronto, Ontario, Canada, M5C 1T4 (http://www.acdlabs.com/).

As shown in Scheme 1, the compounds of Formula I can be prepared under a variety of conditions by sequential reaction of an R⁶-amine and an aryl amine (e.g., Ar—NH—R⁵) with an activated pyrimidine. The order of the synthetic steps may be varied to arrive at the targeted compound. Additional synthetic manipulation of the functionality present in the amine moieties, as shown in Schemes 2-6, allows for further analog generation.

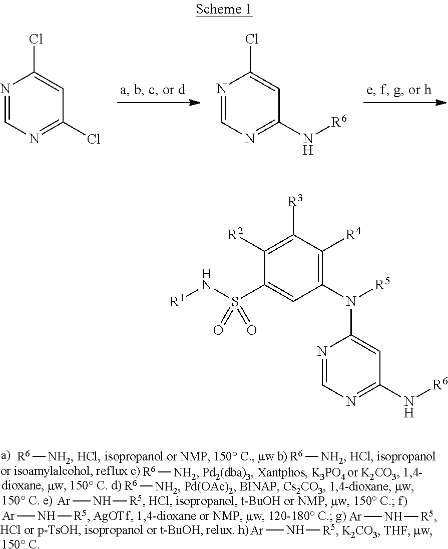

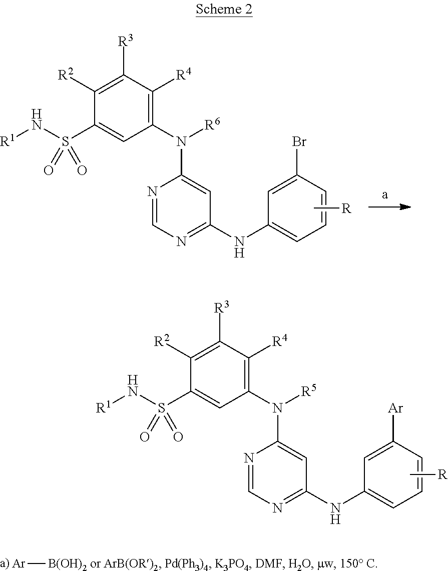

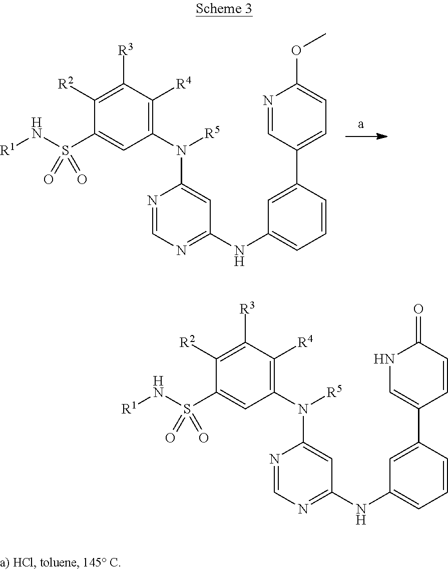

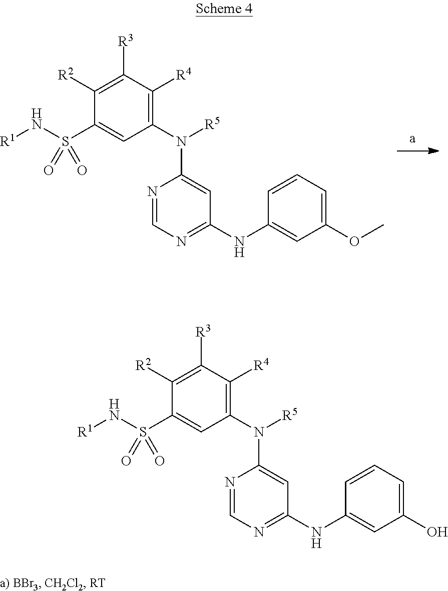

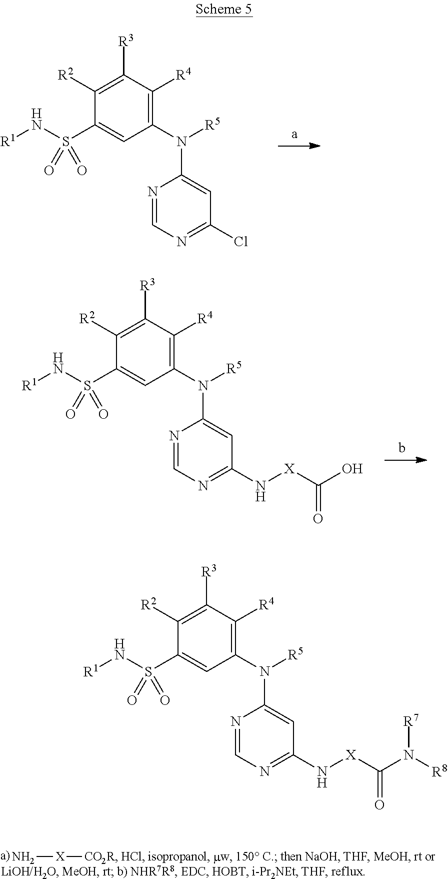

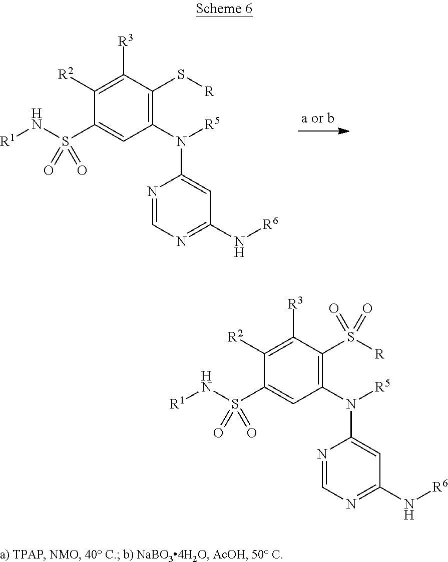

The invention also includes various deuterated forms of the compounds of Formula I. Each available hydrogen atom attached to a carbon atom may be independently replaced with a deuterium atom. A person of ordinary skill in the art will know how to synthesize deuterated forms of the compounds of Formula I. For example, deuterated alkyl group amines may be prepared by conventional techniques (see for example: methyl-d₃-amine available from Aldrich Chemical Co., Milwaukee, Wis., Cat. No. 489, 689-2). Employing such compounds according to Schemes 1-3 will allow for the preparation of compounds of Formula I in which various hydrogen atoms are replaced with a deuterium atom.

### Methods of Use

The present invention is directed to a method of inhibiting TNNI3K which comprises contacting the kinase with a compound of Formula I or a salt thereof, particularly a pharmaceutically acceptable salt thereof. This invention is also directed to a method of treatment of a TNNI3K-mediated disease or disorder comprising administering an effective amount of the compound of Formula I or a salt thereof, particularly a pharmaceutically acceptable salt thereof, to a patient, specifically a human, in need thereof. As used herein, “patient” refers to a human or other mammal. Specifically, this invention is directed to a method of inhibiting TNNI3K activity, comprising contacting the kinase with an effective amount of a compound of Formula I or a pharmaceutically acceptable salt thereof. For example, TNNI3K activity may be inhibited in mammalian cardiac tissue by administering to a patient in need thereof, an effective amount a compound of Formula I or a pharmaceutically acceptable salt thereof.

The compounds of this invention may be particularly useful for treatment of TNNI3K-mediated diseases or disorders, specifically by inhibition of TNNI3K activity, where such diseases or disorders are selected from heart failure, particularly congestive heart failure; cardiac hypertrophy; and heart failure or congestive heart failure resulting from cardiac hypertrophy. The compounds of this invention may also be useful for the treatment of heart failure or congestive heart failure resulting from myocardial ischemia or myocardial infarction.

A therapeutically “effective amount” is intended to mean that amount of a compound that, when administered to a patient in need of such treatment, is sufficient to effect treatment, as defined herein. Thus, e.g., a therapeutically effective amount of a compound of Formula I, or a pharmaceutically acceptable salt thereof, is a quantity of an inventive agent that, when administered to a human in need thereof, is sufficient to modulate or inhibit the activity of TNNI3K such that a disease condition which is mediated by that activity is reduced, alleviated or prevented. The amount of a given compound that will correspond to such an amount will vary depending upon factors such as the particular compound (e.g., the potency (pXC₅₀), efficacy (EC₅₀), and the biological half-life of the particular compound), disease condition and its severity, the identity (e.g., age, size and weight) of the patient in need of treatment, but can nevertheless be routinely determined by one skilled in the art. Likewise, the duration of treatment and the time period of administration (time period between dosages and the timing of the dosages, e.g., before/with/after meals) of the compound will vary according to the identity of the mammal in need of treatment (e.g., weight), the particular compound and its properties (e.g., pharmaceutical characteristics), disease or condition and its severity and the specific composition and method being used, but can nevertheless be determined by one of skill in the art.

“Treating” or “treatment” is intended to mean at least the mitigation of a disease condition in a patient, where the disease condition is caused or mediated by TNNI3K. The methods of treatment for mitigation of a disease condition include the use of the compounds in this invention in any conventionally acceptable manner, for example for prevention, retardation, prophylaxis, therapy or cure of a disease. The compounds of Formula I of this invention may be useful for the treatment of heart failure, particularly congestive heart failure. The compounds of Formula I of this invention may be useful for the treatment of cardiac hypertrophy, and heart failure or congestive heart failure resulting from cardiac hypertrophy, myocardial ischemia or myocardial infarction.

The compounds of the invention may be administered by any suitable route of administration, including both systemic administration and topical administration. Systemic administration includes oral administration, parenteral administration, transdermal administration, rectal administration, and administration by inhalation. Parenteral administration refers to routes of administration other than enteral, transdermal, or by inhalation, and is typically by injection or infusion. Parenteral administration includes intravenous, intramuscular, and subcutaneous injection or infusion. Inhalation refers to administration into the patient's lungs whether inhaled through the mouth or through the nasal passages. Topical administration includes application to the skin.

The compounds of the invention may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for a compound of the invention depend on the pharmacokinetic properties of that compound, such as absorption, distribution, and half-life, which can be determined by the skilled artisan. In addition, suitable dosing regimens, including the duration such regimens are administered, for a compound of the invention depend on the condition being treated, the severity of the condition being treated, the age and physical condition of the patient being treated, the medical history of the patient to be treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change.

Treatment of TNNI3K-mediated disease conditions may be achieved using the compounds of this invention as a monotherapy, or in dual or multiple combination therapy, such as in combination with other cardiovascular agents, for example, in combination with one or more of the following agents: a beta-blocker, an ACE inhibitor, an angiotensin receptor blocker (ARB), a calcium channel blocker, a diuretic, a renin inhibitor, a centrally acting antihypertensive, a dual ACE/NEP inhibitor, an aldosterone synthase inhibitor, and an aldosterone-receptor antagonist, which are administered in effective amounts as is known in the art.

Examples of suitable beta blockers include timolol (such as BLOCARDEN™) carteolol (such as CARTROL™), carvedilol (such as COREG™), nadolol (such as CORGARD™), propanolol (such as INNOPRAN XL™), betaxolol (such as KERLONE™) penbutolol (such as LEVATOL™), metoprolol (such as LOPRESSOR™ and TOPROL-XL™), atenolol (such as TENORMIN™), pindolol (such as VISKEN™), bisoprolol, bucindolol, esmolol, acebutolol, labetalol, nebivolol, celiprolol, sotalol, and oxprenolol. Examples of suitable ACE inhibitors include alacepril, benazepril, benazaprilat, captopril, ceronapril, cilazapril, delapril, enalapril, enalaprilat, fosinopril, lisinopril, moexipiril, moveltopril, perindopril, quinapril, quinaprilat, ramipril, ramiprilat, spirapril, temocapril, trandolapril, and zofenopril. Preferred ACE inhibitors are benazepril, enalpril, lisinopril, and ramipril. Examples of suitable angiotensin receptor blockers include candesartan, eprosartan, irbesartan, losartan, olmesartan, tasosartan, telmisartan, and valsartan. Examples of suitable calcium channel blockers include dihydropyridines (DHPs) and non-DHPs. Suitable DHPs include amlodipine, felodipine, ryosidine, isradipine, lacidipine, nicardipine, nifedipine, nigulpidine, niludipine, nimodiphine, nisoldipine, nitrendipine, and nivaldipine, and their pharmaceutically acceptable salts. Suitable non-DHPs are flunarizine, prenylamine, diltiazem, fendiline, gallopamil, mibefradil, anipamil, tiapamil, and verampimil, and their pharmaceutically acceptable salts. A suitable diuretic is a thiazide derivative selected from amiloride, chlorothiazide, hydrochlorothiazide, methylchlorothiazide, and chlorothalidon. A suitable renin inhibitor is aliskiren. Examples of suitable centrally acting antiphypertensives include clonidine, guanabenz, guanfacine and methyldopa. Examples of suitable dual ACE/NEP inhibitors include omapatrilat, fasidotril, and fasidotrilat. Examples of suitable aldosterone synthase inhibitors include anastrozole, fadrozole, and exemestane. Examples of suitable aldosterone-receptor antagonists include spironolactone and eplerenone.

The invention further includes the use of compounds of the invention as an active therapeutic substance, in particular in the treatment of diseases mediated by TNNI3K. Specifically, the invention includes the use of compounds of the invention in the treatment of heart failure, particularly congestive heart failure; cardiac hypertrophy; heart failure or congestive heart failure resulting from cardiac hypertrophy; and heart failure or congestive heart failure resulting from myocardial ischemia or myocardial infarction.

In another aspect, the invention includes the use of compounds of the invention in the manufacture of a medicament for use in the treatment of the above disorders.

### Compositions

The compounds of the invention will normally, but not necessarily, be formulated into a pharmaceutical composition prior to administration to a patient. Accordingly, in another aspect the invention is directed to pharmaceutical compositions comprising a compound of the invention and a pharmaceutically-acceptable excipient.

The pharmaceutical compositions of the invention may be prepared and packaged in bulk form wherein an effective amount of a compound of the invention can be extracted and then given to the patient such as with powders, syrups, and solutions for injection. Alternatively, the pharmaceutical compositions of the invention may be prepared and packaged in unit dosage form. For oral application, for example, one or more tablets or capsules may be administered. A dose of the pharmaceutical composition contains at least a therapeutically effective amount of a compound of this invention (i.e., a compound of Formula I or a salt, particularly a pharmaceutically acceptable salt, thereof). When prepared in unit dosage form, the pharmaceutical compositions may contain from 1 mg to 1000 mg of a compound of this invention.

The pharmaceutical compositions of the invention typically contain one compound of the invention. However, in certain embodiments, the pharmaceutical compositions of the invention contain more than one compound of the invention. In addition, the pharmaceutical compositions of the invention may optionally further comprise one or more additional pharmaceutically active compounds.

As used herein, “pharmaceutically-acceptable excipient” means a material, composition or vehicle involved in giving form or consistency to the composition. Each excipient must be compatible with the other ingredients of the pharmaceutical composition when commingled such that interactions which would substantially reduce the efficacy of the compound of the invention when administered to a patient and interactions which would result in pharmaceutical compositions that are not pharmaceutically-acceptable are avoided. In addition, each excipient must of course be of sufficiently high purity to render it pharmaceutically-acceptable.

The compounds of the invention and the pharmaceutically-acceptable excipient or excipients will typically be formulated into a dosage form adapted for administration to the patient by the desired route of administration. Conventional dosage forms include those adapted for (1) oral administration such as tablets, capsules, caplets, pills, troches, powders, syrups, elixirs, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as aerosols and solutions; and (6) topical administration such as creams, ointments, lotions, solutions, pastes, sprays, foams, and gels.

Suitable pharmaceutically-acceptable excipients will vary depending upon the particular dosage form chosen. In addition, suitable pharmaceutically-acceptable excipients may be chosen for a particular function that they may serve in the composition. For example, certain pharmaceutically-acceptable excipients may be chosen for their ability to facilitate the production of uniform dosage forms. Certain pharmaceutically-acceptable excipients may be chosen for their ability to facilitate the production of stable dosage forms. Certain pharmaceutically-acceptable excipients may be chosen for their ability to facilitate the carrying or transporting the compound or compounds of the invention once administered to the patient from one organ, or portion of the body, to another organ, or portion of the body. Certain pharmaceutically-acceptable excipients may be chosen for their ability to enhance patient compliance.

Suitable pharmaceutically-acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anti-caking agents, humectants, chelating agents, plasticizers, viscosity increasing agents, antioxidants, preservatives, stabilizers, surfactants, and buffering agents. The skilled artisan will appreciate that certain pharmaceutically-acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically-acceptable excipients in appropriate amounts for use in the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

In one aspect, the invention is directed to a solid oral dosage form such as a tablet or capsule comprising an effective amount of a compound of the invention and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesium stearate, calcium stearate, and talc.

### EXAMPLES

The following examples illustrate the invention. These examples are not intended to limit the scope of the present invention, but rather to provide guidance to the skilled artisan to prepare and use the compounds, compositions, and methods of the present invention. While particular embodiments of the present invention are described, the skilled artisan will appreciate that various changes and modifications can be made without departing from the spirit and scope of the invention.

In the following experimental descriptions, the following abbreviations may be used:

| Abbreviation | Meaning |
|---|---|
| AcOH | acetic acid |
| AgOTf | silver trifluoromethanesulfonate |
| aq. | aqueous |
| BINAP | (R)-(+)-(1,1′-binaphthalene-2,2′- |
| | diyl)bis(diphenylphosphine) |
| brine | saturated aqueous sodium chloride |
| CHO | formaldehyde |
| CH₂Cl₂ | methylene chloride |
| CH₃CN | acetonitrile |
| CH₃NH₂ | methylamine |
| CH₃NH₂•HCl | methylamine hydrochloride |
| CH₃SNa | sodium methyl mercaptide |
| CuCl | copper(I) chloride |
| DDQ | 2,3-dichloro-5,6-dicyanobenzoquinone |
| DMF | N,N-dimethylformamide |
| DMSO | dimethylsulfoxide |
| dppf | 1,1′-bis(diphenylphosphino)ferrocene |
| EDC | 1-[3-(dimethylamino)propyl]-3-ethylcarbodiimide |
| | hydrochloride |
| Et₃N | triethylamine |
| Et₂O | diethyl ether |
| EtOAc | ethyl acetate |
| h | hour(s) |
| HCl | hydrochloric acid |
| HCO₂H | formic acid |
| HOBt | 1-hydroxybenzotriazole |
| H₂SO₄•SO₃ | fuming sulfuric acid |
| i-Pr₂NEt | N,N-diisopropylethylamine |
| KOAc | potassium acetate |
| K₃PO₄ | potassium phosphate tribasic |
| LCMS | liquid chromatography-mass spectroscopy |
| LiOH | lithium hydroxide |
| MeOH | methanol |
| MgSO₄ | magnesium sulfate |
| min | minute(s) |
| MS | mass spectrum |
| μw | microwave |
| NaH | sodium hydride |
| NaHCO₃ | sodium bicarbonate |
| NaOH | sodium hydroxide |
| Na₂SO₄ | sodium sulfate |
| NH₄Cl | ammonium chloride |
| HCO₂•NH₄ | ammonium formate |
| NH₄OH | ammonium hydroxide |
| NMO | 4-methylmorpholine N-oxide |
| NMP | N-methyl-2-pyrrolidone |
| Pd/C | palladium on carbon |
| Pd₂(dba)₃ | tris(dibenzylideneacetone)dipalladium(0) |
| Pd(dppf)Cl₂ | [1,1′-bis(diphenylphosphino)ferrocene] |
| | dichloropalladium(II) |
| Pd(Ph₃)₄ | tetrakis(triphenylphosphine)palladium(0) |
| Ph | phenyl |
| POCl₃ | phosphoryl chloride |
| rt | room temperature |
| satd. | saturated |
| SCX | strong cation exchange |
| TBAB | tetrabutyl ammonium bromide |
| TFA | trifluoroacetic acid |
| THF | tetrahydrofuran |
| TPAP | tetrapropylammonium perruthenate |
| t_{R} | retention time |

### Preparation 1

### N-methyl-3-(4,4,5,5-tetramethyl-1,3,2-dioxaborolan-2-yl)benzenesulfonamide

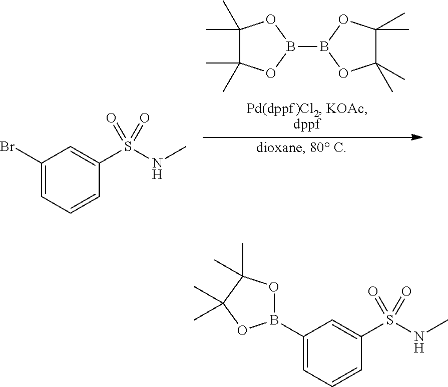

A mixture of 3-bromo-N-methylbenzenesulfonamide (2.3 g, 9.0 mmol), bis(pinacolato)diboron (2.5 g, 10.0 mmol), Pd(dppf)Cl₂ (0.725 g, 0.9 mmol), KOAc (2.6 g, 27 mmol), and dppf (0.700 g, 1.26 mmol) in 1,4-dioxane was heated to 80° C. and stirred overnight under nitrogen. In the morning, the reaction mixture was filtered and concentrated in vacuo. The crude product was then purified via flash column chromatography (4:1 petroleum ether/EtOAc) to give N-methyl-3-(4,4,5,5-tetramethyl-1,3,2-dioxaborolan-2-yl)benzenesulfonamide as a white solid (1.7 g, 65%).

### Preparation 2

### 3-amino-4-fluoro-N-methylbenzenesulfonamide

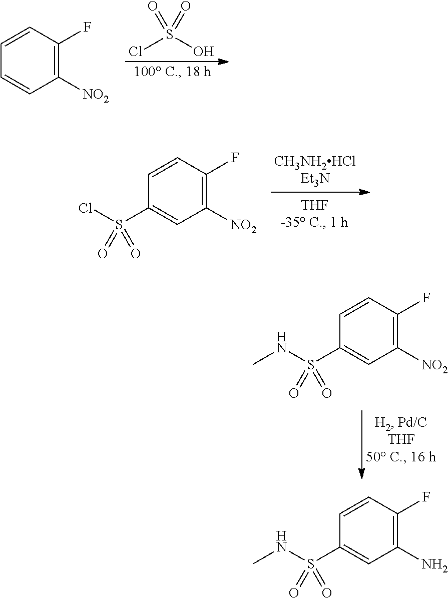

### Step 1. 4-fluoro-3-nitrobenzenesulfonyl chloride

1-Fluoro-2-nitrobenzene (50.0 g, 0.354 mol) was added to chlorosulfonic acid (91 g, 0.778 mol) at 65° C. The resulting mixture was then heated to 100° C. for 18 h. The mixture was cooled to rt, poured over ice and extracted with CH₂Cl₂. The combined organic layers were then washed with NaHCO₃, then brine, dried over MgSO₄, filtered and concentrated in vacuo to afford 4-fluoro-3-nitrobenzenesulfonyl chloride (55.3 g, 65%) as a brown oil.

### Step 2. 4-fluoro-N-methyl-3-nitrobenzenesulfonamide

To a solution of 4-fluoro-3-nitrobenzenesulfonyl chloride (43 g, 179.5 mmol) in THF (500 mL), was added Et₃N (150 mL, 1.08 mol). The mixture was cooled to −35° C. and CH₃NH₂.HCl (14.5 g, 215.4 mmol) in water was added dropwise. After 1 h, the mixture was warmed to rt and diluted with 1:1 water/EtOAc. The organic layer was separated and washed with satd. aq. NaHCO₃, then brine, dried over MgSO₄, filtered and concentrated in vacuo. The crude residue was purified via flash column chromatography (20% EtOAc/petroleum ether) to give 4-fluoro-N-methyl-3-nitrobenzenesulfonamide (38 g, 90%) as a yellow solid.

### Step 3. 3-amino-4-fluoro-N-methylbenzenesulfonamide

To a mixture of 4-fluoro-N-methyl-3-nitrobenzenesulfonamide (1.6 g, 6.83 mmol) in THF (50 mL) under nitrogen, Pd/C (0.600 g) was added. The flask was then evacuated and recharged with hydrogen. The resulting mixture was allowed to stir under a hydrogen atmosphere overnight at 50° C. The mixture was then filtered and concentrated to afford 3-amino-4-fluoro-N-methylbenzenesulfonamide (1.25 g, 89%) as an off-white solid. ¹H NMR (400 MHz, DMSO-d₆) δ 7.26 (q, J=4.85 Hz, 1H), 7.13-7.22 (m, 2H), 6.90 (ddd, J=2.38, 4.27, 8.41 Hz, 1H), 5.63 (s, 2H), 2.40 (d, J=5.02 Hz, 3H); MS (m/z) 205.1 (M+H)⁺.

### Preparation 3

### 3-amino-N-methyl-4-[(1-methylethyl)oxy]benzenesulfonamide

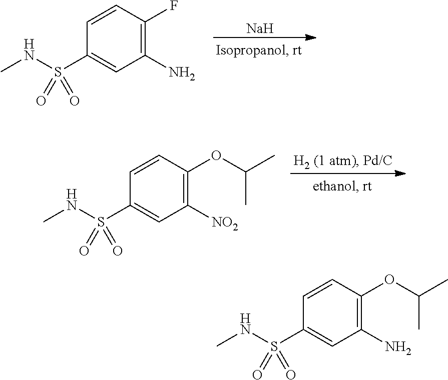

### Step 1. N-methyl-4-[(1-methylethyl)oxy]-3-nitrobenzenesulfonamide

NaH (0.440 g, 11 mmol) was added to 20 mL of isopropanol and the resulting mixture stirred at rt. After 30 min, 4-fluoro-N-methyl-3-nitrobenzenesulfonamide (2.34 g, 10 mmol) was added. The reaction mixture was then stirred at rt overnight. The mixture was poured into EtOAc and water. The organic phase was separated, dried over Na₂SO₄, and concentrated in vacuo to give the crude product. Purification via flash column chromatography (1:1 petroleum ether/EtOAc) afforded N-methyl-4-[(1-methylethyl)oxy]-3-nitrobenzenesulfonamide (1.6 g, 58%) as a yellow solid. MS (m/z) 274.7 (M+H)⁺.

### Step 2. 3-amino-N-methyl-4-[(1-methylethyl)oxy]benzenesulfonamide

To a mixture of N-methyl-4-[(1-methylethyl)oxy]-3-nitrobenzenesulfonamide (1.6 g, 5.8 mmol) in ethanol (20 mL) under nitrogen, Pd/C (0.160 g) was added. The flask was then evacuated and recharged with hydrogen three times. The resulting mixture was allowed to stir under a hydrogen atmosphere overnight at rt. The mixture was then filtered and concentrated to afford 3-amino-N-methyl-4-[(1-methylethyl)oxy]benzenesulfonamide (1.1 g, 77%) as a white solid. ¹H NMR (400 MHz, DMSO-d₆) δ 7.01-7.10 (m, 2H), 6.87-6.98 (m, 2H), 5.08 (br. s., 2H), 4.63 (dt, J=5.93, 11.98 Hz, 1H), 2.34-2.41 (m, 3H), 1.29 (d, J=6.02 Hz, 6H); MS (m/z) 244.7 (M+H)⁺.

The following anilines were prepared from 4-fluoro-N-methyl-3-nitrobenzenesulfonamide using the procedures analogous to those described in Preparation 3:

| | Conditions for | MS | |
|---|---|---|---|
| Aniline Product | Step 1 | (m/z) | ¹H NMR |
| 3-amino-N-methyl-4- | sodium | 217.0 (M + H)⁺ | ¹H NMR (400 MHz, DMSO- |
| (methyloxy)benzenesulfonamide | methoxide, | | d₆) δ ppm 7.09 (q, J = 4.85 Hz, |
| | MeOH | | 1H), 7.03 (s, 1H), |
| | | | 6.94 (s, 2H), 5.18 (s, 2H), 3.83 (s, |
| | | | 3H), 2.36 (d, J = 5.02 Hz, |
| | | | 3H) |
| 3-amino-4-(ethyloxy)-N- | sodium | 231.0 (M + H)⁺ | ¹H NMR (400 MHz, DMSO- |
| methylbenzenesulfonamide | ethoxide, | | d₆) δ ppm 7.06 (q, J = 5.07 Hz, |
| | ethanol | | 1 H), 7.01 (s, 1 H), |
| | | | 6.89 (s, 2 H), 5.12 (s, 2 H), |
| | | | 4.05 (q, J = 6.91 Hz, 2 H), 2.34 (d, |
| | | | J = 5.07 Hz, 3 H), 1.34 (t, |
| | | | J = 6.95 Hz, 3 H) |
| 3-amino-N-methyl-4- | NaH, 1- | 245.1 (M + H)⁺ | ¹H NMR (400 MHz, CHCl₃-d) |
| (propyloxy)benzenesulfonamide | propanol | | δ ppm 7.23 (dd, J = 8.38, 2.21 Hz, |
| | | | 1 H), 7.16 (d, J = 2.21 Hz, |
| | | | 1 H), 6.83 (d, J = 8.38 Hz, 1 |
| | | | H), 4.17 (m, 1 H), 4.03 (t, |
| | | | J = 6.51 Hz, 4 H), 2.64 (d, |
| | | | J = 5.51 Hz, 3 H), |
| | | | 1.83-1.91 (m, 2 H), 1.08 (t, J = 7.39 Hz, |
| | | | 3 H) |
| 3-amino-N-methyl-4-[(2- | NaH, 2-methyl- | 259.0 (M + H)⁺ | ¹H NMR (400 MHz, DMSO- |
| methylpropyl)oxy]benzenesulfonamide | 1-propanol | | d₆) δ ppm 7.06 (q, J = 5.15 Hz, |
| | | | 1 H), 7.01 (d, J = 1.54 Hz, |
| | | | 1 H), 6.85-6.92 (m, 2 H), |
| | | | 5.11 (s, 2 H), 3.77 (d, J = 6.39 Hz, |
| | | | 2 H), 2.34 (d, J = 5.07 Hz, |
| | | | 3 H), 2.00-2.08 (m, 1 H), |
| | | | 0.99 (d, J = 6.62 Hz, 6 H) |
| 3-amino-4-[(1,2- | NaH, 3-methyl- | 273.1 (M + H)⁺ | ¹H NMR (400 MHz, CHCl₃-d) |
| dimethylpropyl)oxy]-N- | 2-butanol | | δ ppm 7.22 (dd, J = 8.36, 2.20 Hz, |
| methylbenzenesulfonamide | | | 1 H), 7.17 (d, J = 2.35 Hz, |
| | | | 1 H), 6.82 (d, J = 8.51 Hz, 1 |
| | | | H), 4.27 (m, 2 H), 4.01 (br. |
| | | | s., 2 H), 2.65 (d, J = 5.58 Hz, |
| | | | 3 H), 2.00 (m, 1 H), 1.29 (d, |
| | | | J = 6.16 Hz, 3 H), 1.00 (d, |
| | | | J = 6.75 Hz, 3 H), 1.03 (d, |
| | | | J = 6.75 Hz, 3 H) |
| 3-amino-4-[(1-ethylpropyl)oxy]-N- | NaH, 3- | 273.1 (M + H)⁺ | ¹H NMR (400 MHz, DMSO- |
| methylbenzenesulfonamide | pentanol | | d₆) δ ppm 7.05 (q, J = 5.07 Hz, |
| | | | 1 H), 7.01 (d, J = 2.21 Hz, |
| | | | 1 H), 6.90 (s, 1 H), 6.89 (d, |
| | | | J = 1.98 Hz, 1 H), 5.07 (s, 2 |
| | | | H), 4.26 (m, 1 H), 2.35 (d, |
| | | | J = 5.07 Hz, 3 H), |
| | | | 1.58-1.66 (m, 4 H), 0.88 (t, J = 7.39 Hz, |
| | | | 6 H) |
| 3-amino-N-methyl-4-[(2,2,2- | NaH, 2,2,2- | 285.0 (M + H)⁺ | ¹H NMR (400 MHz, DMSO- |
| trifluoroethyl)oxy]benzenesulfonamide | trifluoroethanol | | d₆) δ ppm 7.16 (q, J = 4.85 Hz, |
| | | | 1 H), 7.03-7.10 (m, 2 |
| | | | H), 6.91 (dd, J = 8.38, 2.21 Hz, |
| | | | 1 H), 5.23 (s, 2 H), |
| | | | 4.79 (q, J = 8.82 Hz, 2 H), 2.35 (d, |
| | | | J = 5.07 Hz, 3 H) |
| 3-amino-N-methyl-4-[(3,3,3- | NaH, 3,3,3- | 299.0 (M + H)⁺ | ¹H NMR (400 MHz, DMSO- |
| trifluoropropyl)oxy]benzenesulfonamide | trifluoro-1- | | d₆) δ ppm 7.08 (m, 1 H), |
| | propanol | | 7.01 (d, J = 2.21 Hz, 1 H), |
| | | | 6.93-6.98 (m, 1 H), |
| | | | 6.90 (m, 2 H), 5.10 (s, 2 H), |
| | | | 4.21 (t, J = 5.95 Hz, 2 H), |
| | | | 2.77-2.84 (m, 2 H), 2.33 (d, |
| | | | J = 4.63 Hz, 3 H) |
| 3-amino-4-(cyclopentyloxy)-N- | NaH, | 271.1 (M + H)⁺ | ¹H NMR (400 MHz, DMSO- |
| methylbenzenesulfonamide | cyclopentanol | | d₆) δ ppm 7.04 (q, J = 4.85 Hz, |
| | | | 1 H), 7.00 (d, J = 1.76 Hz, |
| | | | 1 H), 6.86-6.90 (m, 2 H), |
| | | | 5.07 (br. s., 2 H), 4.83 (m, 1 |
| | | | H), 2.34 (d, J = 5.07 Hz, 3 H), |
| | | | 1.89 (m, 2 H), |
| | | | 1.69-1.77 (m, 4 H), 1.55-1.62 (m, 2 |
| | | | H) |
| 3-amino-4-(cyclohexyloxy)-N- | NaH, | 285.1 (M + H)⁺ | ¹H NMR (400 MHz, DMSO- |
| methylbenzenesulfonamide | cyclohexanol | | d₆) δ ppm 7.52 (s, 1 H), 7.38 |
| | | | m, 2 H), 7.23 (d, J = 8.82 Hz, |
| | | | 1 H), 4.51 (br. s., 1 H), |
| | | | 2.37 (s, 3 H), 1.89 (m, 2 H), |
| | | | 1.73 (m, 2 H), 1.51 (m, 3 H), |
| | | | 1.37 (m, 3 H) |
| 3-amino-N-methyl-4-[(2,2,2- | NaH, | 298.9 (M + H)⁺ | ¹H NMR (400 MHz, DMSO- |
| trifluoro-1-methylethyl)oxy]benzenesulfonamide | 1,1,1-trifluoro- | | d₆) δ ppm 7.14-7.22 (m, 2 |
| | 2-propanol | | H), 7.11 (d, J = 2.26 Hz, 1 H), |
| | | | 6.92 (dd, J = 8.41, 2.38 Hz, 1 |
| | | | H), 5.19-5.30 (m, 3 H), |
| | | | 2.39 (d, J = 5.02 Hz, 3 H), |
| | | | 1.45 (d, J = 6.27 Hz, 3 H) |

The following anilines were prepared from 1,1-dimethylethyl[(4-fluoro-3-nitrophenyl)sulfonyl]methylcarbamate using the procedures analogous to those described in Preparation 3:

| | Conditions for | | |
|---|---|---|---|
| Aniline Product | Step 1 | MS (m/z) | Comment |
| 1,1-dimethylethyl ({3-amino-4- | NaH, 1,1,1- | 312.8 (M + H)⁺ | Isolated as a mixture of |
| [(2,2,2-trifluoro-1,1- | trifluoro-2- | deprotected | protected and |
| dimethylethyl)oxy]phenyl}sulfonyl)methylcarbamate | methyl-2- | 356.9 (M − tBu)⁺ | deprotected material. |
| | propanol | | |
| 1,1-dimethylethyl [(3-amino-4- | NaH, | 397.0 (M − tBu)⁺ | |
| {[2,2,2-trifluoro-1- | 1,1,1,3,3,3- | | |
| (trifluoromethyl)ethyl]oxy}phenyl)sulfonyl]methylcarbamate | hexafluoro-2- | | |
| | propanol | | |

### Preparation 4

### 3-amino-N-methyl-4-(4-morpholinyl)benzenesulfonamide

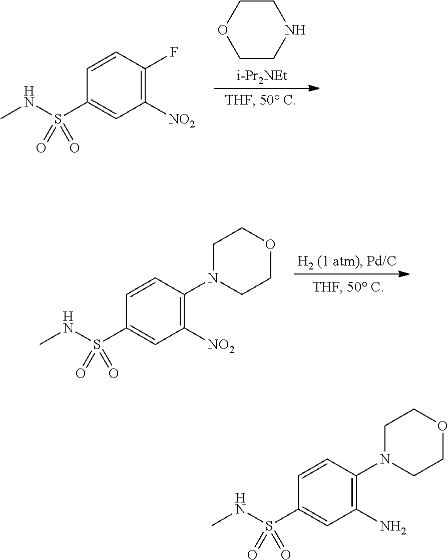

### Step 1. N-methyl-4-(4-morpholinyl)-3-nitrobenzenesulfonamide

To a solution of 4-fluoro-N-methyl-3-nitrobenzenesulfonamide (2.00 g, 8.54 mmol) and morpholine (0.744 g, 8.54 mmol) in THF (100 mL), was added i-Pr₂NEt (2.21 g, 17.08 mmol). The resulting solution was stirred at 50° C. overnight. In the morning, the reaction mixture was cooled to rt and concentrated to dryness in vacuo. The residue was dissolved in EtOAc and washed with water and brine, dried over MgSO₄, filtered and concentrated in vacuo to obtain N-methyl-4-(4-morpholinyl)-3-nitrobenzenesulfonamide (2.5 g, 97%) as a red oil. MS (m/z) 302.0 (M+H)⁺.

### Step 2. 3-amino-N-methyl-4-(4-morpholinyl)benzenesulfonamide

To a mixture of N-methyl-4-(4-morpholinyl)-3-nitrobenzenesulfonamide (2.5 g, 8.30 mmol) in THF (100 mL) under nitrogen, Pd/C (0.8 g) was added. The flask was then evacuated and recharged with hydrogen three times. The resulting mixture was allowed to stir under a hydrogen atmosphere at 50° C. overnight. The mixture was then filtered and concentrated to afford 3-amino-N-methyl-4-(4-morpholinyl)benzenesulfonamide (1.98 g, 88%). ¹H NMR (400 MHz, DMSO-d₆) δ 7.07-7.17 (m, 2H), 7.01 (d, J=8.28 Hz, 1H), 6.94 (dd, J=1.88, 8.16 Hz, 1H), 5.20 (s, 2H), 3.72-3.81 (m, 4H), 2.80-2.89 (m, 4H), 2.38 (d, J=4.77 Hz, 3H); MS (m/z) 272.2 (M+H)⁺.

The following anilines were prepared from 4-fluoro-N-methyl-3-nitrobenzenesulfonamide and the indicated amine using the procedures analogous to those described in Preparation 4:

| | Conditions for | MS | |
|---|---|---|---|
| Aniline Product | Step 1 | (m/z) | ¹H NMR |
| 3-amino-4-(dimethylamino)-N- | DIPEA, | 230.2 (M + H)⁺ | ¹H NMR (400 MHz, DMSO- |
| methylbenzene-sulfonamide | dimethylamine | | d₆) δ 7.03-7.10 (m, 2H), |
| | | | 7.00 (d, J = 8.28 Hz, 1H), |
| | | | 6.93 (dd, J = 2.13, 8.16 Hz, |
| | | | 1H), 5.13 (s, 2H), 2.62 (s, |
| | | | 6H), 2.38 (d, J = 5.02 Hz, |
| | | | 3H) |
| 3-amino-4-[ethyl(methyl)amino]- | DIPEA, | 244.1 (M + H)⁺ | ¹H NMR (400 MHz, DMSO- |
| N-methylbenzene-sulfonamide | ethyl(methyl)amine | | d₆) δ 7.06-7.13 (m, 2H), |
| | | | 7.02 (d, J = 8.28 Hz, 1H), |
| | | | 6.93 (dd, J = 1.76, 8.03 Hz, |
| | | | 1H), 5.11 (s, 2H), 2.89 (q, J = 7.03 Hz, |
| | | | 2H), 2.60 (s, 3H), |
| | | | 2.39 (d, J = 5.02 Hz, 3H), |
| | | | 1.03 (t, J = 7.03 Hz, 3H) |
| 3-amino-4-(diethylamino)-N- | DIPEA, | 258.0 (M + H)⁺ | ¹H NMR (400 MHz, DMSO- |
| methylbenzenesulfonamide | diethylamine | | d₆) δ ppm 0.93 (t, J = 7.03 Hz, |
| | | | 6 H) 2.40 (d, J = 5.02 Hz, 3 |
| | | | H) 2.95 (q, J = 7.03 Hz, 4 H) |
| | | | 5.15 (s, 2 H) 6.92 (dd, |
| | | | J = 8.03, 2.01 Hz, 1 H) |
| | | | 7.01-7.17 (m, 3 H) |
| 3-amino-N-methyl-4-(2-methyl-1- | No base, | 270.1 (M + H)⁺ | ¹H NMR (400 MHz, DMSO- |
| pyrrolidinyl)benzenesulfonamide | 2-methylpyrrolidine | | d₆) δ ppm 0.91 (d, J = 6.02 Hz, |
| | | | 3 H) 1.43-1.54 (m, 1 H) |
| | | | 1.68-1.81 (m, 1 H) |
| | | | 1.84-1.95 (m, 1 H) |
| | | | 2.09-2.18 (m, |
| | | | 1 H) 2.38 (d, J = 4.77 Hz, 3 |
| | | | H) 2.52-2.58 (m, 1 H) |
| | | | 3.56-3.70 (m, 2 H) 5.04 (s, 2 H) |
| | | | 6.89-6.98 (m, 2 H) |
| | | | 7.04-7.12 (m, 2 H) |
| 3-amino-4-(2,5-dimethyl-1- | No base, | 284.0 (M + H)⁺ | ¹H NMR (400 MHz, DMSO- |
| pyrrolidinyl)-N- | 2,5- | | d₆) δ ppm 0.88 (d, J = 6.02 Hz, |
| methylbenzenesulfonamide | dimethylpyrrolidine | | 6 H) 1.43-1.56 (m, 2 H) |
| | | | 1.95-2.06 (m, 2 H) 2.41 (s, |
| | | | 3 H) 3.09 (d, J = 5.52 Hz, 2 |
| | | | H) 5.38 (s, 2 H) 6.92 (dd, |
| | | | J = 8.16, 2.13 Hz, 1 H) |
| | | | 7.09 (d, J = 2.26 Hz, 1 H) 7.19 (s, |
| | | | 1 H) 7.29 (d, J = 8.28 Hz, 1 |
| | | | H) |
| 3-amino-N-methyl-4-[2- | Et₃N, 2- | 324.0 (M + H)⁺ | ¹H NMR (400 MHz, DMSO- |
| (trifluoromethyl)-1- | (trifluoromethyl)pyrrolidine | | d₆) δ ppm 1.86-2.04 (m, 3 |
| pyrrolidinyl]benzenesulfonamide | | | H) 2.27-2.38 (m, 1 H) |
| | | | 2.65-2.75 (m, 1 H) |
| | | | 3.49-3.58 (m, 1 H) 4.47 (br. s., 1 H) |
| | | | 5.20 (s, 2 H) 6.91 (dd, |
| | | | J = 8.28, 2.26 Hz, 1 H) |
| | | | 7.10 (d, J = 2.26 Hz, 1 H) 7.16 (br. |
| | | | s., 1 H) 7.31 (d, J = 8.28 Hz, |
| | | | 1 H) |
| 3-amino-4-(3,3-difluoro-1- | Et₃N, 3,3- | 306.0 (M + H)⁺ | ¹H NMR (400 MHz, DMSO- |
| piperidinyl)-N- | difluoropiperidine | | d₆) δ ppm 1.80-1.89 (m, 2 |
| methylbenzenesulfonamide | | | H) 1.98-2.10 (m, 2 H) |
| | | | 2.39 (s, 3 H) 2.85-2.92 (m, 2 H) |
| | | | 3.14 (t, J = 11.29 Hz, 2 H) |
| | | | 5.11 (s, 2 H) 6.96 (dd, |
| | | | J = 8.28, 2.26 Hz, 1 H) |
| | | | 7.06 (d, J = 8.28 Hz, 1 H) 7.13 (d, |
| | | | J = 2.26 Hz, 1 H) 7.18 (s, 1 H) |
| 3,4-diamino-N- | Ammonia (7M in | 202.0 (M + H)⁺ | ¹H NMR (400 MHz, DMSO- |
| methylbenzenesulfonamide | MeOH) | | d₆) δ ppm 2.33 (s, 3 H) |
| | | | 4.84 (s, 2 H) 5.22 (s, 2 H) 6.56 (d, |
| | | | J = 8.03 Hz, 1 H) |
| | | | 6.77-6.86 (m, 2 H) 6.90 (d, J = 1.76 Hz, |
| | | | 1 H) |

### Preparation 5

### 3-amino-N-methyl-4-(methylthio)benzenesulfonamide

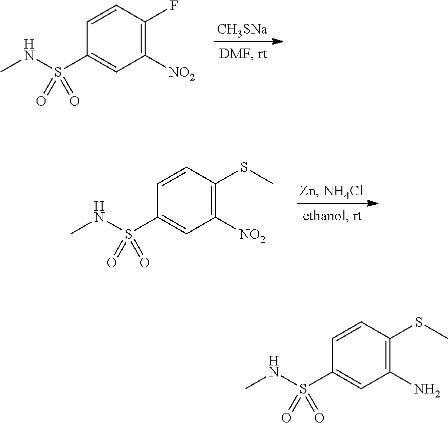

### Step 1. N-methyl-4-(methylthio)-3-nitrobenzenesulfonamide

To a solution of 4-fluoro-N-methyl-3-nitrobenzenesulfonamide (15 g, 64.01 mmol) in THF (150 mL), was added 20% CH₃SNa (22.4 g, 64.01 mmol) dropwise. The resulting mixture was then stirred overnight. In the morning, the mixture was poured into EtOAc and water, the organic phase separated, dried over Na₂SO₄, filtered and concentrated. The crude material was then purified via flash column chromatography (1:1 EtOAc/petroleum ether) to afford N-methyl-4-(methylthio)-3-nitrobenzenesulfonamide (3.29 g, 19%) as a yellow solid. MS (m/z) 262.7 (M+H)⁺.

### Step 2. 3-amino-N-methyl-4-(methylthio)benzenesulfonamide

To a solution of N-methyl-4-(methylthio)-3-nitrobenzenesulfonamide (1.0 g, 3.81 mmol) in 10 mL of ethanol and 10 mL of NH₄Cl, zinc dust (2.5 g, 3.81 mmol) was added. The reaction mixture was stirred overnight at rt. The mixture was then filtered and diluted with EtOAc and water. The organic phase was separated, washed with water and brine, dried over MgSO₄, filtered and concentrated to afford 3-amino-N-methyl-4-(methylthio)benzenesulfonamide (0.500 g, 56%) as a white solid. ¹H NMR (400 MHz, DMSO-d₆) δ 7.06 (d, J=8.03 Hz, 1H), 6.86 (s, 1H), 6.67-6.76 (m, 1H), 5.28 (br. s., 2H), 2.17 (s, 3H), 2.21 (s, 3H); MS (m/z) 232.7 (M+H)⁺.

### Preparation 6

### 3-amino-4-(ethylthio)-N-methylbenzenesulfonamide

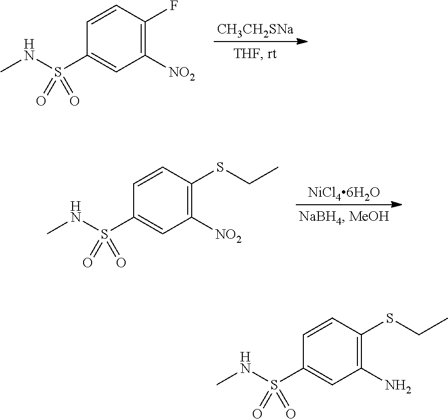

### Step 1: 4-(ethylthio)-N-methyl-3-nitrobenzenesulfonamide

Sodium ethyl thiolate (1.08 g, 12.8 mmol) was added to a mixture of 4-fluoro-N-methyl-3-nitrobenzenesulfonamide (2 g, 8.6 mmol) in THF (20 mL) and the mixture stirred at rt for 5 h. Water was added to the reaction and extracted with EtOAc. The organic phases were combined, dried (Na₂SO₄) and concentrated to give 4-(ethylthio)-N-methyl-3-nitrobenzenesulfonamide (2.0 g, 85%) as a yellow solid. MS (m/z) 276.9 (M+H)⁺.

### Step 2: 3-amino-4-(ethylthio)-N-methylbenzenesulfonamide

Sodium borohydride (1.1 g, 29 mmol) was added to a mixture of 4-(ethylthio)-N-methyl-3-nitrobenzenesulfonamide (2.0 g, 7.3 mmol) and nickel (II) chloride hexahydrate (3.4 g, 14.5 mmol) in MeOH (20 mL) and the mixture stirred for 5 min at 0° C. The MeOH was then removed and the residual solid suspended in CH₂Cl₂, filtered and the filtrate concentrated to give 3-amino-4-(ethylthio)-N-methylbenzenesulfonamide (1.5 g, 84%) as a yellow solid. ¹H NMR (400 MHz, DMSO-d₅) δ ppm 1.16 (t, J=7.28 Hz, 3H) 2.38 (d, J=4.85 Hz, 3H) 2.85 (q, J=7.28 Hz, 2H) 5.60 (br. s, 2H) 6.87 (dd, J=7.94, 1.98 Hz, 1H) 7.08 (d, J=1.98 Hz, 1H) 7.26 (q, J=5.07 Hz, 1H) 7.33 (d, J=8.16 Hz, 1H); MS (m/z) 246.9 (M+H)⁺.

The following anilines were prepared from 4-fluoro-N-methyl-3-nitrobenzenesulfonamide and the indicated thiol using the procedures described in Preparation 6:

| Aniline Product | Thiol | MS (m/z) | ¹H NMR |
|---|---|---|---|
| 3-amino-N-methyl-4-[(1- | i-PrSH | 261.0 (M + H)⁺ | ¹H NMR (400 MHz, DMSO-d₆) |
| methylethyl)thio]benzenesulfonamide | | | δ ppm 1.17 (d, J = 6.62 Hz, 6 H) |
| | | | 2.39 (d, J = 5.07 Hz, 3 H) |
| | | | 3.28-3.36 (m, 1 H) 5.69 (s, 2 H) |
| | | | 6.84 (dd, J = 7.94, 1.98 Hz, 1 H) |
| | | | 7.10 (d, J = 2.20 Hz, 1 H) 7.26 (q, |
| | | | J = 5.07 Hz, 1 H) 7.36 (d, J = 7.94 Hz, |
| | | | 1 H) |
| 3-amino-N-methyl-4-[(2- | i-PrCH₂SH | 275.1 (M + H)⁺ | ¹H NMR (400 MHz, DMSO-d₆) δ |
| methylpropyl)thio]benzenesulfonamide | | | ppm 0.94 (d, J = 6.62 Hz, 6 H) |
| | | | 1.62-1.74 (m, 1 H) 2.36 (d, |
| | | | J = 5.29 Hz, 3 H) 2.71 (d, J = 6.62 Hz, |
| | | | 2 H) 5.58 (s, 2 H) 6.85 (dd, |
| | | | J = 8.16, 1.98 Hz, 1 H) 7.06 (d, |
| | | | J = 1.76 Hz, 1 H) 7.23 (q, J = 4.85 Hz, |
| | | | 1 H) 7.32 (d, J = 8.38 Hz, 1 |
| | | | H) |
| 3-amino-4-[(1,1- | t-BuSH | 274.9 (M + H)⁺ | ¹H NMR (400 MHz, DMSO-d₆) δ |
| dimethylethyl)thio]-N- | | Major ion is | ppm 1.23 (s, 9 H) 2.38 (d, |
| methylbenzenesulfonamide | | 218.9 (M − tBu)⁺ | J = 4.85 Hz, 3 H) 5.87 (s, 2 H) |
| | | | 6.81 (dd, 1 H) 7.12 (d, J = 1.98 Hz, |
| | | | 1 H) 7.31 (q, J = 4.78 Hz, 1 |
| | | | H) 7.36 (d, J = 7.94 Hz, 1 H) |
| 3-amino-N-methyl-4-[(2,2,2- | CF₃CH₂SH | 300.7 (M + H)⁺ | ¹H NMR (400 MHz, DMSO-d₆) δ |
| trifluoroethyl)thio]benzenesulfonamide | | | ppm 2.39 (d, J = 5.07 Hz, 3 H) |
| | | | 3.72 (q, J = 10.36 Hz, 2 H) |
| | | | 5.87 (s, 2 H) 6.85 (dd, J = 8.05, 2.09 Hz, |
| | | | 1 H) 7.14 (d, J = 1.98 Hz, 1 |
| | | | H) 7.33 (q, 1 H) 7.48 (d, J = 7.94 Hz, |
| | | | 1 H) |

### Preparation 7

### 3-amino-4-hydroxy-N-methylbenzenesulfonamide

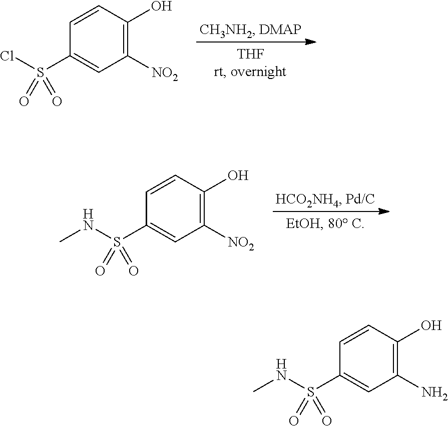

### Step 1. 4-hydroxy-N-methyl-3-nitrobenzenesulfonamide

A suspension of 4-hydroxy-3-nitrobenzenesulfonyl chloride (0.749 g, 3.15 mmol) and DMAP (0.077 g, 0.630 mmol) in THF (7.880 mL) was treated with CH₃NH₂ (2 M in THF, 6.30 mL, 12.61 mmol). The resulting mixture was then stirred at rt overnight. The mixture was then filtered and the filtrate partitioned between CH₂Cl₂ and satd. aq. NaHCO₃. The layers were separated by hydrophobic frit. The aq. layer was then extracted at pH 7, pH 5 (twice), and pH 2. The pH 5 and pH 2 extracts were then combined and concentrated to afford 4-hydroxy-N-methyl-3-nitrobenzenesulfonamide (0.311 g, 42%) as a pale yellow solid. ¹H NMR (400 MHz, DMSO-d₆) δ 12.09 (br. s., 1H), 8.22 (d, J=2.52 Hz, 1H), 7.88 (dd, J=2.27, 8.81 Hz, 1H), 7.53 (q, J=4.95 Hz, 1H), 7.31 (d, J=8.81 Hz, 1H), 2.42 (d, J=5.04 Hz, 3H); MS (m/z) 232.8 (M+H)⁺.

### Step 2. 3-amino-4-hydroxy-N-methylbenzenesulfonamide

A solution of 4-hydroxy-N-methyl-3-nitrobenzenesulfonamide (0.280 g, 1.206 mmol) in ethanol (0.269 mL) was added to a mixture of HCO₂.NH₄ (0.380 g, 6.03 mmol) and Pd/C (0.128 g, 0.121 mmol) in ethanol (0.269 mL) and the reaction heated to 80° C. Once the reaction mixture reached 80° C., it was allowed to cool to rt and stand overnight. The mixture was then filtered through Celite® and concentrated to give 3-amino-4-hydroxy-N-methylbenzenesulfonamide (0.177 g, 73%) as a brown oil. ¹H NMR (400 MHz, DMSO-d₆) δ 9.88 (br. s., 1H), 7.00 (d, J=2.01 Hz, 2H), 6.80-6.87 (m, 1H), 6.75 (d, J=8.28 Hz, 1H), 4.97 (br. s., 2H), 2.35 (d, J=4.77 Hz, 3H); MS (m/z) 202.9 (M+H)⁺.

### Preparation 8

### 3-amino-4-chloro-N-methylbenzenesulfonamide

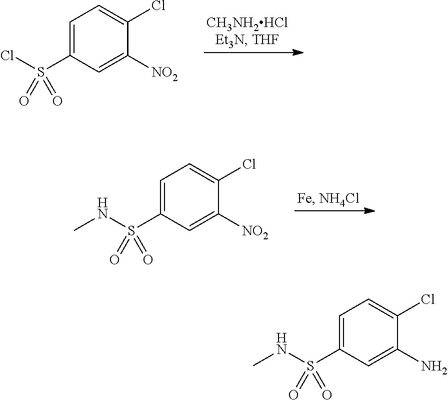

### Step 1. 4-chloro-N-methyl-3-nitrobenzenesulfonamide

A solution of 4-chloro-3-nitrobenzenesulfonyl chloride (10 g, 39.1 mmol) in THF (100 mL) was cooled to −40° C. before being treated with a solution of CH₃NH₂.HCl (2.64 g, 39.1 mmol) in 10 mL of water followed by TEA (5.44 mL, 39.1 mmol). The reaction mixture was stirred and allowed to warm to rt over 1 h before being partitioned between 350 mL EtOAc and 30 mL brine. The organic layer was washed twice with brine, dried over MgSO₄ and subjected to flash chromatography (330 g silica gel, 0-40% EtOAc/hexane) to afford 4-chloro-N-methyl-3-nitrobenzenesulfonamide (6.38 g, 65%) as a light yellow solid. MS (m/z) 251.0 (M+H)⁺.

### Step 2. 3-amino-4-chloro-N-methylbenzenesulfonamide

A solution of 4-chloro-N-methyl-3-nitrobenzenesulfonamide (6.35 g, 25.3 mmol) in EtOH (150 mL) and water (50.0 mL) was treated with iron (14.15 g, 253 mmol) and NH₄Cl (13.55 g, 253 mmol) and heated at 90° C. for 4 h before being cooled and filtered through Celite®. The filter cake was washed with EtOAc and the combined filtrate was filtered again to remove precipitated NH₄Cl before being concentrated. The resulting crude material was partitioned between 350 mL EtOAc and 50 mL saturated aq. NaHCO₃. The organic layer was washed with brine, dried over MgSO₄, concentrated and subjected to flash column chromatography (330 g silica gel, 0-15% EtOAc/CH₂Cl₂) to afford 3-amino-4-chloro-N-methylbenzenesulfonamide (5.604 g, 100%) as a light yellow crystalline solid. ¹H NMR (400 MHz, MeOD) δ ppm 7.39 (d, J=8.28 Hz, 1H), 7.27 (d, J=2.26 Hz, 1H), 7.03 (dd, J=8.28, 2.26 Hz, 1H), 2.54 (s, 3H). MS 221.0 (M+H)⁺.

The following aniline was prepared using the stated sulfonyl chloride and procedures analogous to those described in Preparation 7 and 8:

| | Sulfonyl chloride | Conditions | |
|---|---|---|---|
| | and base in | for | MS |
| Aniline Product | Step 1 | Step 2 | (m/z) |
| 3-amino-N,4- | 4-methyl-3- | HCO₂•NH₄, | 201.0 |
| dimethyl- | nitrobenzenesulfonyl | Pd/C | (M + H)⁺ |
| benzenesulfonamide | chloride, Et₃N | | |

### Preparation 9

### 3-amino-N-methyl-4-[methyl(2,2,2-trifluoroethyl)amino]benzenesulfonamide

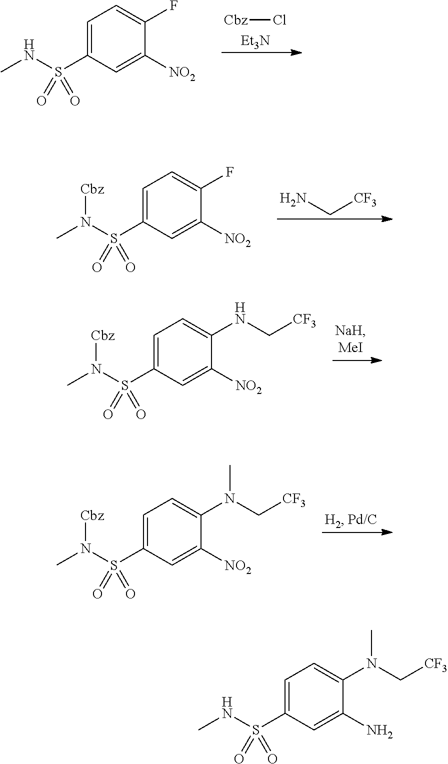

### Step 1. phenylmethyl[(4-fluoro-3-nitrophenyl)sulfonyl]methylcarbamate

A solution of 4-fluoro-N-methyl-3-nitrobenzenesulfonamide (2 g, 8.54 mmol) in THF (20 mL) was treated with Et₃N (2.380 mL, 17.08 mmol) followed by dropwise addition of benzyl chloroformate (3.75 mL, 11.10 mmol). The mixture was stirred at 25° C. for 5 h before being concentrated. The residue was treated with water and extracted with CH₂Cl₂. The organic extracts were washed (brine), dried (Na₂SO₄), concentrated, and subjected to flash chromatography (25-50% EtOAc-hexanes) to give a yellow solid, which was suspended in EtOAc-hexanes, collected by filtration, and washed with hexanes to give phenylmethyl[(4-fluoro-3-nitrophenyl)sulfonyl]methylcarbamate (1 g, 32%) as a white solid. MS (m/z) 391.0 (M+Na)⁺.

### Step 2. phenylmethyl methyl({3-nitro-4-[(2,2,2-trifluoroethyl)amino]phenyl}sulfonyl) carbamate

A solution of phenylmethyl[(4-fluoro-3-nitrophenyl)sulfonyl]methylcarbamate (1 g, 2.71 mmol) in THF (10 mL) at 25° C. was treated with 2,2,2-trifluoroethylamine (0.592 g, 5.97 mmol) and stirred for 20 h before being concentrated to give a yellow oil, which was dissolved in EtOAc/hexanes. A yellow precipitate formed, which was collected by filtration and washed with hexanes to give phenylmethyl methyl({3-nitro-4-[(2,2,2-trifluoroethyl)amino]phenyl}sulfonyl)carbamate (1.07 g, 88%) as a yellow solid. MS (m/z) 448.1 (M+H)⁺.

### Step 3. phenylmethyl methyl({4-[methyl(2,2,2-trifluoroethyl)amino]-3-nitrophenyl}sulfonyl) carbamate

A solution of phenylmethyl methyl({3-nitro-4-[(2,2,2-trifluoroethyl)amino]phenyl}sulfonyl)carbamate (1 g, 2.24 mmol) in DMF (1 mL) at 25° C. was treated with NaH (0.179 g, 4.47 mmol) and stirred for 2 min before being treated with iodomethane (0.42 mL, 6.71 mmol). After 1 h, the mixture was diluted with water and extracted with EtOAc. The organic extract was washed (brine), dried (Na₂SO₄), concentrated, and subjected to flash chromatography (10-35% EtOAc-hexanes) to give phenylmethyl methyl({4-[methyl(2,2,2-trifluoroethyl)amino]-3-nitrophenyl}sulfonyl)carbamate (539 mg, 52%) as a yellow oil. MS (m/z) 462.1 (M+H)⁺.

### Step 4. 3-amino-N-methyl-4-[methyl(2,2,2-trifluoroethyl)amino]benzenesulfonamide

A solution of phenylmethyl methyl({4-[methyl(2,2,2-trifluoroethyl)amino]-3-nitrophenyl}sulfonyl)carbamate (539 mg, 1.17 mmol) in MeOH (10 mL) at 25° C. was treated with 10% Pd/C (124 mg, 0.117 mmol) and stirred under an atmosphere of hydrogen (balloon) overnight before being filtered through Celite®. The filtrate was again filtered through a 0.45 micron syringe filter and concentrated to give 3-amino-N-methyl-4-[methyl(2,2,2-trifluoroethyl)amino]benzenesulfonamide (320 mg, 92%) as a brown oil. ¹H NMR (400 MHz, DMSO-d₆) δ ppm 7.14-7.20 (m, 2H), 7.12 (d, J=2.26 Hz, 1H), 6.95 (dd, J=8.28, 2.26 Hz, 1H), 5.23 (s, 2H), 3.82 (q, J=9.87 Hz, 2H), 2.83 (s, 3H), 2.39 (d, J=5.02 Hz, 3H). MS (m/z) 298.0 (M+H)⁺.

### Preparation 10

### 5-amino-2-fluoro-N-methylbenzenesulfonamide

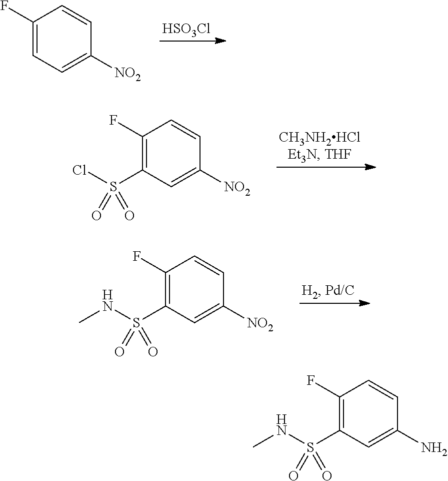

### Step 1. 2-fluoro-5-nitrobenzenesulfonyl chloride

A mixture of 1-fluoro-4-nitrobenzene (3.0 g, 21.3 mmol) in chlorosulfonic acid (5.5 mL, 84 mmol) was stirred at 90-100° C. for 8 h before being cooled to rt and slowly poured into ice water and extracted with EtOAc. The organic extract was washed with saturated aq. NaHCO₃ and water, dried (Na₂SO₄), and concentrated to give 2-fluoro-5-nitrobenzenesulfonyl chloride (3.2 g, 63%) as a colorless oil, which was used directly in the next step.

### Step 2. 2-fluoro-N-methyl-5-nitrobenzenesulfonamide

A solution of 2-fluoro-5-nitrobenzenesulfonyl chloride (3.2 g, 12.6 mmol) in THF (30 mL) at −45° C. was treated with methylamine hydrochloride (1.0 g, 15.1 mmol) and Et₃N (2.1 mL, 15.1 mmol) and stirred for 30 min. The mixture was then treated with 6M aq. HCl to adjust the pH to 3 and warmed to rt before being diluted with water and extracted with EtOAc. The organic extract was dried (Na₂SO₄), concentrated, and subjected to flash chromatography (5-20% EtOAc-petroleum ether) to give 2-fluoro-N-methyl-5-nitrobenzenesulfonamide as a yellow solid (3.0 g, 93%). MS (m/z) 235.1 (M+H)⁺.

### Step 3. 5-amino-2-fluoro-N-methylbenzenesulfonamide

A solution of 2-fluoro-N-methyl-5-nitrobenzenesulfonamide (3.0 g, 12.8 mmol) in MeOH (40 mL) was treated with 10% Pd/C (300 mg, 0.28 mmol) and stirred under hydrogen (40 psi) for 8 h before being filtered through Celite® and concentrated to give 5-amino-2-fluoro-N-methylbenzenesulfonamide (2.5 g, 96%) as an off-white solid. ¹H NMR (400 MHz, DMSO-d₆) δ ppm 7.40-7.49 (m, 1H), 7.01-7.09 (m, 1H), 6.94 (dd, J=5.95, 2.87 Hz, 1H), 6.71-6.77 (m, 1H), 5.49 (br. s., 2H), 2.45 (d, J=4.85 Hz, 3H). MS (m/z) 205.1 (M+H)⁺.

The following anilines were prepared from the indicated nitrobenzenes using procedures analogous to those described in Preparation 10:

| | Nitrobenzene | MS | |
|---|---|---|---|
| Aniline Product | in Step 1 | (m/z) | ¹H NMR |
| 3-amino-N-methyl-4- | 1-nitro-2- | 271.0 (M + H)⁺ | ¹H NMR (400 MHz, DMSO-d₆) δ |
| [(trifluoromethyl)oxy]benzenesulfonamide | [(trifluoromethyl)oxy]benzene | | ppm 7.39 (q, J = 4.77 Hz, 1 H), |
| | | | 7.31 (dd, J = 8.53, 1.51 Hz, 1 H), |
| | | | 7.24 (d, J = 2.26 Hz, 1 H), |
| | | | 6.92 (dd, J = 8.41, 2.38 Hz, 1 H), |
| | | | 5.92 (s, 2 H), 2.43 (d, J = 4.77 Hz, 3 H) |
| 5-amino-2-fluoro-N-methyl-4- | 4-fluoro-2- | 235.1 (M + H)⁺ | ¹H NMR (400 MHz, DMSO-d₆) δ |
| (methyloxy)benzenesulfonamide | (methyloxy)-1- | | ppm 7.31 (br. s., 1 H), 6.96 (d, |
| | nitrobenzene | | J = 7.28 Hz, 1 H), 6.90 (d, |
| | | | J = 11.91 Hz, 1 H), 4.97 (s, 2 H), |
| | | | 3.82 (s, 3 H), 2.40 (d, J = 3.75 Hz, |
| | | | 3 H) |

### Preparation 11

### 5-amino-4-(dimethylamino)-2-fluoro-N-methylbenzenesulfonamide

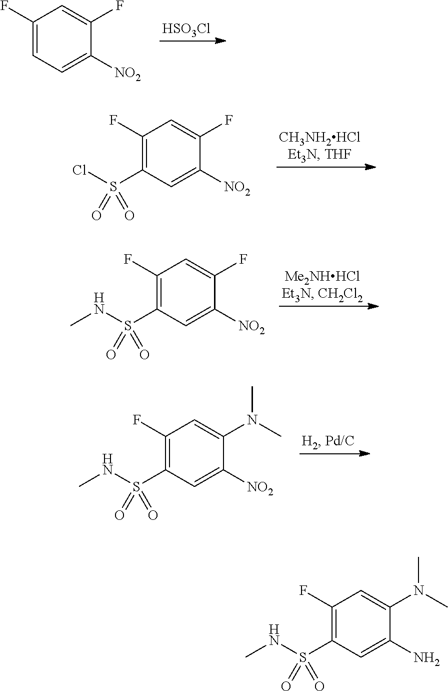

### Step 1. 2,4-difluoro-5-nitrobenzenesulfonyl chloride

A mixture of 2,4-difluoro-1-nitrobenzene (20 g, 126 mmol) in chlorosulfonic acid (44 g, 378 mmol) was stirred at 100° C. for 48 h before being poured into ice-water and extracted with EtOAc. The organic extract was dried (Na₂SO₄) and concentrated, and the residue was triturated with 10% EtOAc-petroleum ether to give 2,4-difluoro-5-nitrobenzenesulfonyl chloride as a brown oil (21 g, 81%) which was used directly in the next step.

### Step 2. 2,4-difluoro-N-methyl-5-nitrobenzenesulfonamide

A solution of 2,4-difluoro-5-nitrobenzenesulfonyl chloride (21 g, 81 mmol) in THF (400 mL) at −60° C. was treated with methylamine hydrochloride (6.6 g, 97 mmol) and then treated dropwise with Et₃N (22.6 mL, 162 mmol). After stirring for 6 h at −60 to −40° C. the mixture was adjusted to pH 3 with the addition of 15% aq. HCl, diluted with water, and extracted with EtOAc. The organic extracts were dried (Na₂SO₄), concentrated, and subjected to flash chromatography (17% EtOAc-petroleum ether) to give 2,4-difluoro-N-methyl-5-nitrobenzenesulfonamide (8 g, 38%) as a brown solid.

¹H NMR (400 MHz, CDCl₃) δ ppm 8.66-8.74 (m, 1H), 7.20-7.25 (m, 1H), 4.81-4.91 (m, 1H), 2.78-2.81 (m, 3H).

### Step 3. 4-(dimethylamino)-2-fluoro-N-methyl-5-nitrobenzenesulfonamide

A solution of 2,4-difluoro-N-methyl-5-nitrobenzenesulfonamide (8.0 g, 31.6 mmol) in CH₂Cl₂ (200 mL) at −20° C. was treated with dimethylamine hydrochloride (2.56 g, 31.6 mmol). The resulting mixture was treated dropwise with Et₃N and stirred for 1 h before being treated with 15% aq. HCl to adjust the pH, diluted with water, and extracted with EtOAc. The organic extract was dried (Na₂SO₄), concentrated, and subjected to flash chromatography (20-50% EtOAc-petroleum ether) to give 4-(dimethylamino)-2-fluoro-N-methyl-5-nitrobenzenesulfonamide (4.0 g, 46%) as a yellow solid. MS (m/z) 278.1 (M+H)⁺.

### Step 4. 5-amino-4-(dimethylamino)-2-fluoro-N-methylbenzenesulfonamide

A solution of 4-(dimethylamino)-2-fluoro-N-methyl-5-nitrobenzenesulfonamide (4.0 g, 14.3 mmol) in MeOH (100 mL) was treated with 10% Pd/C (400 mg) and stirred under H₂ (50 psi) for 16 h before being filtered, concentrated, and subjected to flash chromatography (33-50% EtOAc-petroleum ether) to give 5-amino-4-(dimethylamino)-2-fluoro-N-methylbenzenesulfonamide as a white solid (2.5 g, 71%). ¹H NMR (400 MHz, CDCl₃) δ ppm 7.13 (d, J=7.28 Hz, 1H), 6.75 (d, J=11.69 Hz, 1H), 4.58 (q, J=4.85 Hz, 1H), 3.87 (br. s., 2H), 2.66 (d, J=5.51 Hz, 3H). MS (m/z) 248.1 (M+H)⁺.

### Preparation 12

### 5-amino-2-fluoro-N-methyl-4-(methylthio)benzenesulfonamide

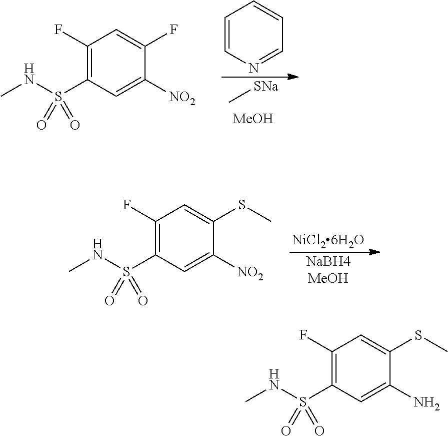

### Step 1: 2-fluoro-N-methyl-4-(methylthio)-5-nitrobenzenesulfonamide

A mixture of 2,4-difluoro-N-methyl-5-nitrobenzenesulfonamide (2 g, 7.9 mmol) and pyridine (1.25 g, 15.9 mmol) in MeOH (1 mL) was cooled to 0° C. Sodium methanethiolate (21%, 2.92 g, 8.6 mmol) was then added slowly and the mixture stirred at 0° C. for 30 min. The reaction was then diluted by the addition of CH₂Cl₂. The organic was separated and washed with brine, dried (Na₂SO₄) and then concentrated. The crude was combined with another batch of material and recrystallised from CH₂Cl₂/petroleum ether to give 5-amino-2-fluoro-N-methyl-4-(methylthio)benzenesulfonamide as a yellow solid. MS (m/z) 281.0 (M+H)⁺.

### Step 2: 5-amino-2-fluoro-N-methyl-4-(methylthio)benzenesulfonamide

To a solution of 2-fluoro-N-methyl-4-(methylthio)-5-nitrobenzenesulfonamide (3 g, 10.7 mmol) in MeOH at 0° C. was added nickel (II) chloride hexahydrate (5.04 g, 21.4 mmol) and sodium borohydride (1.62 g, 42.8 mmol). After 5 min the MeOH was removed, water added to the residue and the solution extracted with CH₂Cl₂. The CH₂Cl₂ was then dried (Na₂SO₄) and concentrated. The residue was combined with that from another batch and purified via flash chromatography (silica gel, 5:1 petroleum ether:EtOAc) to give 5-amino-2-fluoro-N-methyl-4-(methylthio)benzenesulfonamide (50% over two batches) as a white solid. MS (m/z) 251.1 (M+H)⁺.

### Preparation 13

### 5-amino-2-fluoro-N-methyl-4-[(2,2,2-trifluoroethyl)oxy]benzenesulfonamide

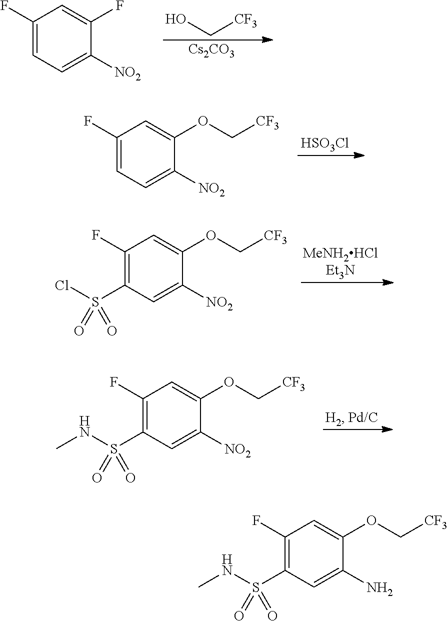

### Step 1. 4-fluoro-1-nitro-2-[(2,2,2-trifluoroethyl)oxy]benzene

A mixture of 2,4-difluoro-1-nitrobenzene (10 g, 62.9 mmol) and 2,2,2-trifluoroethanol (6.29 g, 62.9 mmol) in THF (100 mL) at 25° C. was treated with Cs₂CO₃ (20.5 g, 62.9 mmol) and stirred for 8 h before being diluted with the addition of water and extracted with EtOAc. The organic extract was dried (Na₂SO₄), concentrated, and subjected to flash chromatography (3% EtOAc-petroleum ether) to give 4-fluoro-1-nitro-2-[(2,2,2-trifluoroethyl)oxy]benzene (10 g, 67%) as a yellow solid. MS (m/z) 240.0 (M+H)⁺.

### Step 2. 2-fluoro-5-nitro-4-[(2,2,2-trifluoroethyl)oxy]benzenesulfonyl chloride

A mixture of 4-fluoro-1-nitro-2-[(2,2,2-trifluoroethyl)oxy]benzene (10 g, 41.8 mmol) in chlorosulfonic acid (82 mL, 125.5 mmol) was stirred at 50° C. for 8 h before being poured into ice and extracted with EtOAc. The organic extracts were dried (Na₂SO₄) and concentrated to give 2-fluoro-5-nitro-4-[(2,2,2-trifluoroethyl)oxy]benzenesulfonyl chloride (15 g, crude) as a brown oil, which was used directly in the next step.

### Step 3. 2-fluoro-N-methyl-5-nitro-4-[(2,2,2-trifluoroethyl)oxy]benzenesulfonamide

A mixture of 2-fluoro-5-nitro-4-[(2,2,2-trifluoroethyl)oxy]benzenesulfonyl chloride (15 g, crude) in THF (150 mL) at −45° C. was treated with methylamine hydrochloride (5.96 g, 89 mmol) and then treated dropwise with Et₃N (12.4 mL, 89 mmol). After stirring for 1 h at −45° C., the mixture was adjusted to pH 3 by the addition of aq. 3M HCl, warmed to rt, diluted with water, and extracted with EtOAc. The organic extract was dried (Na₂SO₄), concentrated, and subjected to flash chromatography (9-17% EtOAc-petroleum ether) to give 2-fluoro-N-methyl-5-nitro-4-[(2,2,2-trifluoroethyl)oxy]benzenesulfonamide (10 g, 72% for two steps) as a yellow solid. MS (m/z) 333.0 (M+H)⁺.

### Step 4. 5-amino-2-fluoro-N-methyl-4-[(2,2,2-trifluoroethyl)oxy]benzenesulfonamide

A mixture of 2-fluoro-N-methyl-5-nitro-4-[(2,2,2-trifluoroethyl)oxy]benzene-sulfonamide (10 g, 30.1 mmol) in MeOH (150 mL) was treated with 10% Pd/C (1 g) and stirred under H₂ (45 psi) at 45° C. for 10 h before being filtered. The filtrate was concentrated to give 5-amino-2-fluoro-N-methyl-4-[(2,2,2-trifluoroethyl)oxy]benzene-sulfonamide (8 g, 88%) as a white solid. ¹H NMR (400 MHz, DMSO-d₆) δ ppm 7.40 (q, J=5.07 Hz, 1H), 7.10 (d, J=11.69 Hz, 1H), 7.05 (d, J=7.28 Hz, 1H), 5.04 (s, 2H), 4.83 (q, J=8.82 Hz, 2H), 2.42 (d, J=4.41 Hz, 3H). MS (m/z) 303.0 (M+H)⁺.

The following aniline was prepared from 2,4-difluoro-1-nitrobenzene and the indicated alcohol using procedures analogous to those described in Preparation 13:

| Aniline Product | Alcohol in Step 1 | MS (m/z) |
|---|---|---|
| 5-amino-2-fluoro-N-methyl-4- | 1,1,1-trifluoro-2- | 317.0 (M + H)⁺ |
| [(2,2,2-trifluoro-1-methylethyl)oxy- | propanol | |
| ]benzenesulfonamide | | |

### Preparation 14

### 5-amino-N-methyl-3-pyridinesulfonamide

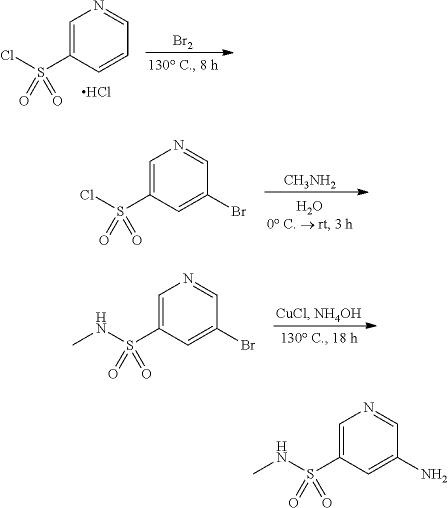

### Step 1. 5-bromo-3-pyridinesulfonyl chloride

A mixture of 3-pyridinesulfonyl chloride hydrochloride (8.9 g, 44 mmol) and bromine (14 g, 88 mmol) was heated to 130° C. for 8 h. The mixture was cooled and used directly in the next step.

### Step 2. 5-bromo-N-methyl-3-pyridinesulfonamide

To CH₃NH₂ (50 mL of a 23-30 weight percent in H₂O) at 0° C., was added 5-bromo-3-pyridinesulfonyl chloride (44 mmol). The mixture was then warmed to rt and stirred for 3 h. The mixture was then extracted with EtOAc and concentrated in vacuo. The crude material was combined with that from an additional experiment (10 mmol scale) run under identical conditions and washed with 10:1 hot petroleum ether/EtOAc to afford 5-bromo-N-methyl-3-pyridinesulfonamide (2.4 g, 18% combined yield over two steps).

### Step 3. 5-amino-N-methyl-3-pyridinesulfonamide

A mixture of 5-bromo-N-methyl-3-pyridinesulfonamide (2.4 g, 9.6 mmol), CuCl (0.100 g, 1.01 mmol), and NH₄OH (5 mL) was heated to 130° C. for 18 h in a sealed tube. The reaction mixture was then treated with sodium sulfide and extracted with EtOAc. The combined organic extracts were then concentrated in vacuo and the crude material washed with 20:5:3 hot petroleum ether/EtOAc/MeOH to afford 5-amino-N-methyl-3-pyridinesulfonamide (1.1 g, 61%) as a brown solid. ¹H NMR (400 MHz, DMSO-d₆) δ 8.11 (d, J=2.51 Hz, 1H), 8.04 (d, J=1.76 Hz, 1H), 7.47 (br. s., 1H), 7.24 (t, J=2.13 Hz, 1H), 5.83 (br. s., 2H), 2.44 (s, 3H); MS (m/z) 188.1 (M+H)⁺.

### Preparation 15

### 3-chloro-N-methyl-5-nitrobenzenesulfonamide

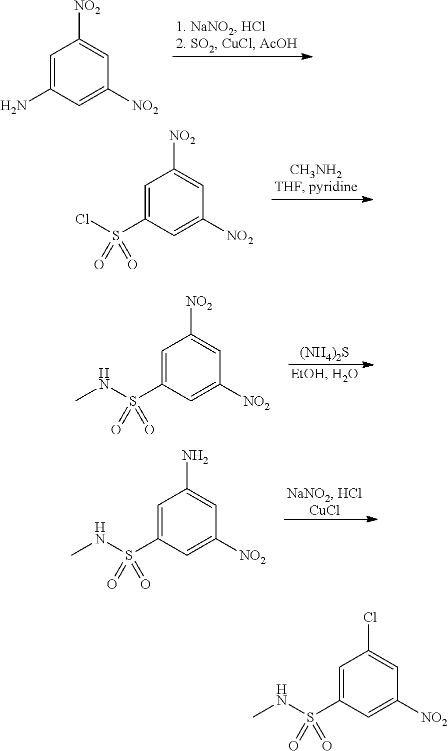

### Step 1. 3,5-dinitrobenzenesulfonyl chloride

(3,5-dinitrophenyl)amine (5 g, 27.3 mmol) was added in one portion to a well stirred solution of concentrated HCl (conc.) (20 mL) and 20 mL water and the mixture was cooled to −10° C. before a solution of NaNO₂ (2.072 g, 30.0 mmol) in water (5 mL) was added dropwise at such a rate that the temperature did not exceed −5° C. The mixture was stirred for 45 min at −10° C. after the addition. While the diazotization reaction proceeded, a separate well-stirred solution of AcOH (6.67 mL) and 30 mL water was saturated with SO₂ by bubbling the gas into the solution until all gas introduced emerged to the surface. CuCl (0.676 g, 6.83 mmol) was added to the solution and the introduction of SO₂ was continued until the yellow-green suspension became blue-green. The SO₂/CuCl mixture was then cooled to 10° C. before being treated with the diazotization reaction mixture in portions over a 20 min period. The foaming that occurred upon addition was disrupted with a few drops of Et₂O. After the addition was complete, the dark red mixture was poured into ice-water (100 mL) and stirred until the ice melted before being filtered. The collected solid was dried in air to afford 3,5-dinitrobenzenesulfonyl chloride (6.01 g, 83%) as a red solid that was used directly in the next step.

### Step 2. N-methyl-3,5-dinitrobenzenesulfonamide

A light brown solution of 3,5-dinitrobenzenesulfonyl chloride (7.28 g, 27.3 mmol) in THF (200 mL) was treated with pyridine (100 mL) to give a dark brown solution, which was cooled to −10° C. before methyl amine (in THF) (13.65 mL, 27.3 mmol) was added slowly by syringe. The resulting solution was stirred at rt for 48 h before being concentrated. The crude residue was partitioned between 600 mL EtOAc and 150 mL 1 N HCl. The organic layer was washed twice with 100 mL 1 N HCl, brine (50 mL), dried over MgSO₄, concentrated, and subjected to flash column chromatography (330 g silica gel, 0-10% EtOAc/CH₂Cl₂) to afford N-methyl-3,5-dinitrobenzenesulfonamide (1.98 g, 28%). ¹H NMR (400 MHz, MeOD) δ ppm 9.20 (s, 1H), 8.96 (d, J=2.01 Hz, 2H), 2.65 (s, 3 H).

### Step 3. 3-amino-N-methyl-5-nitrobenzenesulfonamide

A light red solution of N-methyl-3,5-dinitrobenzenesulfonamide (1.98 g, 7.58 mmol) in ethanol (120 mL) was treated with a solution of ammonium sulfide (2.58 g, 37.9 mmol) in water (15 mL). The resulting dark red solution was heated at 80° C. before being filtered, concentrated, and extracted three times with EtOAc (100 mL). The organic layer was dried over MgSO₄, concentrated, and purified by SCX ion exchange column (20 g×2, washed with MeOH and eluted with 3 M ammonia in MeOH). The appropriate fractions were concentrated to afford a dark brown solid. The aqueous phase contained significant amount of target product, thus, it was concentrated and the residue was re-distributed in 200 mL EtOAc and then concentrated. The resulting brown oil was combined with the above solid and purified by flash column chromatography (120 g silica column, 0-10% MeOH (w/0.1% aq. NH₄OH)/CH₂Cl₂) to afford 3-amino-N-methyl-5-nitrobenzenesulfonamide (0.698 g, 39.8%) as a yellow-brown solid. ¹H NMR (400 MHz, MeOD) δ ppm 7.77 (m, 1H), 7.62-7.69 (m, 1H), 7.40 (m, 1H), 2.58 (s, 3H). MS (m/z) 232.0 (M+H)⁺.

### Step 4. 3-chloro-N-methyl-5-nitrobenzenesulfonamide

3-amino-N-methyl-5-nitrobenzenesulfonamide (0.698 g, 3.02 mmol) was added in one portion into a solution of HCl (conc.) (10 mL, 329 mmol) and 10 mL water and the mixture was cooled to −10° C. before a solution of sodium nitrite (0.208 g, 3.02 mmol) in 5 mL water was added dropwise. The resulting mixture was stirred at −10° C. for 30 min before being added slowly into a mixture of CuCl (0.075 g, 0.755 mmol) in 20 mL of concentrated HCl at 4° C. The reaction mixture was stirred at 0° C. for 15 min before being poured into 150 mL water, filtered, washed with water and dried in air to afford 3-chloro-N-methyl-5-nitrobenzenesulfonamide (0.510 g, 67.4%) as a light brown solid. ¹H NMR (400 MHz, MeOD) δ ppm 8.55 (m, 2H), 8.23 (m, 1H), 2.62 (s, 3H). MS (m/z) 251.0 (M+H)⁺.

### Preparation 16

### 3-amino-5-chloro-N-methylbenzenesulfonamide

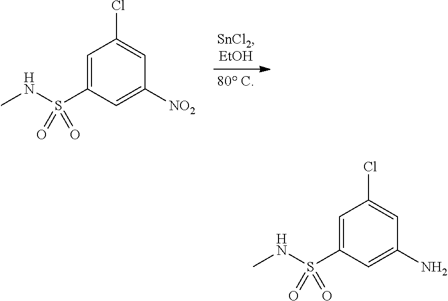

A solution of 3-chloro-N-methyl-5-nitrobenzenesulfonamide (104 mg, 0.415 mmol) in ethanol (10 mL) was treated with tin(II) chloride (315 mg, 1.660 mmol) and heated at 84° C. for 3 h before being concentrated and subjected to flash column chromatography (40 g silica column, 0-100% EtOAc/Hexane) to afford 3-amino-5-chloro-N-methylbenzenesulfonamide (63 mg, 68.8%) as a white solid. ¹H NMR (400 MHz, MeOD) δ ppm 7.00 (d, J=1.76 Hz, 1H), 6.98 (t, J=1.63 Hz, 1H), 6.86 (t, J=1.88 Hz, 1H), 2.55 (s, 3 H). MS (m/z) 221.0 (M+H)⁺.

### Preparation 17

### 3-amino-5-(dimethylamino)-N-methylbenzenesulfonamide

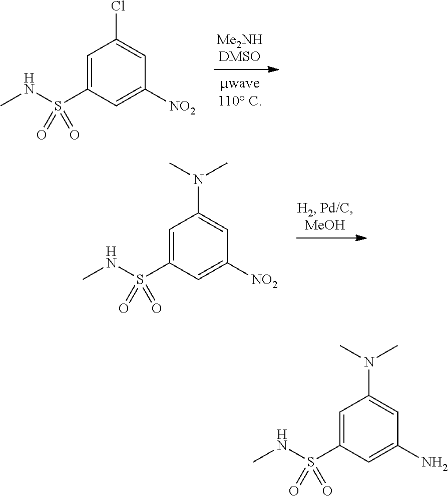

### Step 1. 3-(dimethylamino)-N-methyl-5-nitrobenzenesulfonamide

A mixture of 3-chloro-N-methyl-5-nitrobenzenesulfonamide (150 mg, 0.598 mmol) and dimethylamine (2 M in water) (1.496 mL, 2.99 mmol) in DMSO (4 mL) was heated under microwave irradiation at 110° C. for 30 min before being subjected to reverse phase HPLC (Sunfire 30×100 C-18 column, 10-50% CH₃CN/water (w/0.1% TFA) over 14 min) to afford 69 mg of a light yellow solid. HNMR analysis demonstrated that this solid was 3:1 mixture of starting material and product. Thus, the solid was dissolved in 6 mL DMSO, treated with a solution of dimethylamine (1.5 mL, 2 M aq. solution) and heated at 110° C. for 20 h before being partitioned between 120 mL EtOAc and 20 mL brine. The organic layer was dried over MgSO₄, concentrated, and subjected to flash column chromatography (40 g silica column, 0-40% EtOAc/hexane) to afford 3-(dimethylamino)-N-methyl-5-nitrobenzenesulfonamide (42 mg, 27.1%) as a yellow solid. ¹H NMR (400 MHz, MeOD) δ ppm 7.84 (d, J=1.51 Hz, 1H), 7.70 (d, J=2.01 Hz, 1H), 7.42 (d, J=1.25 Hz, 1H), 3.14 (s, 6H), 2.58 (s, 3H). MS (m/z) 260.0 (M+H)⁺.

### Step 2. 3-amino-5-(dimethylamino)-N-methylbenzenesulfonamide

A solution of 3-(dimethylamino)-N-methyl-5-nitrobenzenesulfonamide (42 mg, 0.162 mmol) in MeOH (15 mL) was purged with nitrogen before being treated with Pd/C (1.724 mg, 0.016 mmol) and then placed under a hydrogen balloon. The mixture was stirred at rt for 4 h before being filtered and concentrated to afford 3-amino-5-(dimethylamino)-N-methylbenzenesulfonamide (38 mg, 0.166 mmol, 102%) as a light brown oil, which was used immediately in the subsequent reaction. MS (m/z) 230.1 (M+H)⁺.

### Preparation 18

### N-methyl-2,3-dihydro-1H-indole-6-sulfonamide

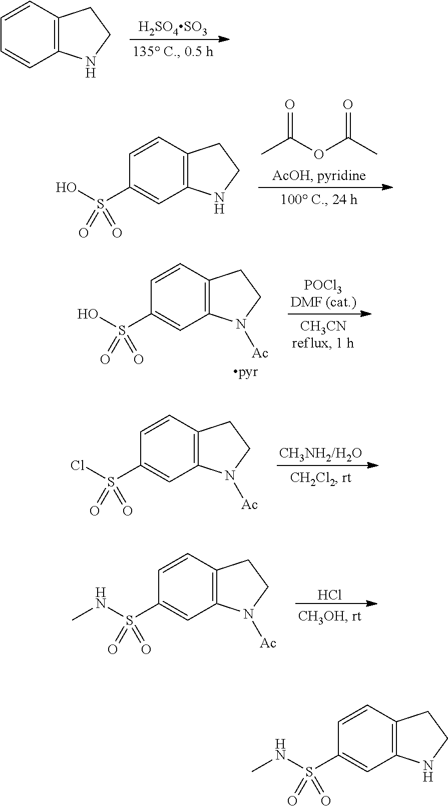

### Step 1. 2,3-dihydro-1H-indole-6-sulfonic acid

H₂SO₄.SO₃ (20%, 21 mL, 0.42 mmol) was cooled to 0° C. Indoline (5.0 g, 0.042 mmol) was added dropwise such that the temperature of the reaction mixture did not rise above 35° C. When the addition was complete the mixture was heated to 135° C. for 0.5 h. After cooling, the solution was poured into an ice bath at which time the product crystallized. The mixture was then filtered and washed with water and acetone to give 2,3-dihydro-1H-indole-6-sulfonic acid (6.9 g, 82%) as a white solid.

### Step 2. 1-acetyl-2,3-dihydro-1H-indole-6-sulfonic acid

To a slurry of 2,3-dihydro-1H-indole-6-sulfonic acid (6.9 g, 34.6 mmol) in AcOH (40 mL), was added acetic anhydride (3.5 g, 34.6 mmol) and pyridine (15 mL). The mixture was then heated to 100° C. for 24 h before it was cooled and concentrated to afford 1-acetyl-2,3-dihydro-1H-indole-6-sulfonic acid (8.8 g, 84%) as a brown oil that was used in the next step without further purification.

### Step 3. 1-acetyl-2,3-dihydro-1H-indole-6-sulfonyl chloride

To a mixture of POCl₃ (12.6 g, 153.33 mmol) and one drop of DMF in CH₃CN (100 mL), was added 1-acetyl-2,3-dihydro-1H-indole-6-sulfonic acid (8.8 g, 27.5 mmol). The mixture was heated to reflux for 1 h and then concentrated to give a pale yellow oil. The oil was then poured into ice and filtered to give 1-acetyl-2,3-dihydro-1H-indole-6-sulfonyl chloride (7.0 g) as a brown solid that was used in the next step without further purification.

### Step 4. 1-acetyl-N-methyl-2,3-dihydro-1H-indole-6-sulfonamide

To a solution of 1-acetyl-2,3-dihydro-1H-indole-6-sulfonyl chloride (7.0 g, 27.0 mmol) in 100 mL of CH₂Cl₂, 30% aq. methyl amine was added dropwise at a rate such that the internal temperature of the reaction did not rise above 22° C. The mixture was then stirred for 2 h. The solution was washed with water, then brine, dried over Na₂SO₄, filtered and concentrated in vacuo. The residue was purified via flash column chromatography (1:1 petroleum ether/EtOAc) to give 1-acetyl-N-methyl-2,3-dihydro-1H-indole-6-sulfonamide (5.0 g, 74%) as a brown solid. MS (m/z) 255.3 (M+H)⁺.

### Step 5. N-methyl-2,3-dihydro-1H-indole-6-sulfonamide

A slurry of 1-acetyl-N-methyl-2,3-dihydro-1H-indole-6-sulfonamide (5.0 g, 19.7 mmol) was purged with HCl gas for 30 min. The solution was then stirred at rt for 2 h before the solution was concentrated in vacuo. The resulting solid was dissolved in satd. aq. NaHCO₃ and EtOAc. The layers were separated and the organic layer washed with water, then brine, dried over Na₂SO₄, filtered and concentrated in vacuo. The crude material was then purified via flash column chromatography (silica gel, 1:1 EtOAc/petroleum ether) to afford N-methyl-2,3-dihydro-1H-indole-6-sulfonamide (1.49 g, 32%) as a yellow solid. ¹H NMR (400 MHz, DMSO-d₆) δ 7.13-7.23 (m, 2H), 6.90 (dd, J=1.51, 7.53 Hz, 1H), 6.77-6.83 (m, 1H), 5.96 (s, 1H), 3.44-3.54 (m, 2H), 2.97 (t, J=8.66 Hz, 2H), 2.37 (d, J=5.02 Hz, 3H); MS (m/z) 255.3 (M+H)⁺.

### Preparation 19

### N,3,3-trimethyl-2,3-dihydro-1H-indole-6-sulfonamide

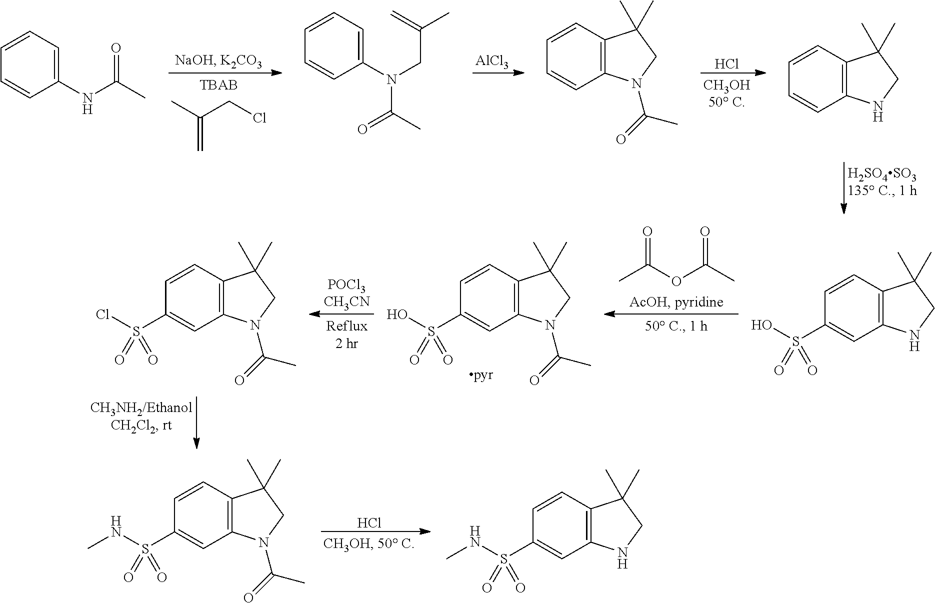

### Step 1. N-(2-methyl-2-propen-1-yl)-N-phenylacetamide

N-phenylacetamide (25.0 g, 185.2 mmol), potassium carbonate (28.1 g, 203.7 mmol), NaOH (8.1 g, 203.7 mmol), TBAB (1.2 g, 3.7 mmol) and toluene (500 mL) were mixed and heated to 75° C. with vigorous stirring. The reaction was stirred for 16 h at 75° C. The mixture was then cooled to rt, water was added and the mixture stirred until all the solids had dissolved. The aqueous layer was separated and the toluene layer washed with 5N HCl and water. The solvent was then removed under reduced pressure to give N-(2-methyl-2-propen-1-yl)-N-phenylacetamide (30 g, 85%) as an oil. MS (m/z) 255.3 (M+H)⁺.

### Step 2. 1-acetyl-3,3-dimethyl-2,3-dihydro-1H-indole

N-(2-methyl-2-propen-1-yl)-N-phenylacetamide (25.0 g, 131 mmol) was added slowly to a stirred suspension of aluminium trichloride (38.0 g, 289 mmol) in chlorobenzene (25 mL) at 115° C. under nitrogen. The temperature was maintained at 115-120° C. for the duration of the addition. The reaction was then stirred for 1 h at 115-120° C. then cooled to rt. Toluene was added and the mixture stirred to give a solution. Water was then slowly added at such a rate to maintain the internal temperature to below 45° C. with cooling applied. The organic layer was separated and washed with 6N HCl and then concentrated to give 1-acetyl-3,3-dimethyl-2,3-dihydro-1H-indole (22.0 g, 88%) as a brown solid. ¹H NMR (400 MHz, CHLOROFORM-d) δ ppm 1.34 (s, 6H) 2.21 (s, 3H) 3.76 (s, 2H) 7.01-7.06 (m, 1H) 7.11 (s, 1H) 7.16-7.22 (m, 1H) 8.17 (d, J=8.16 Hz, 1H)

### Step 3. 3,3-dimethyl-2,3-dihydro-1H-indole

To a solution of 1-acetyl-3,3-dimethyl-2,3-dihydro-1H-indole (22.0 g, 115.8 mmol) in MeOH (100 mL) was added 4M HCl in MeOH (100 mL) and the mixture stirred at 50° C. for 16 h. The solvent was then removed under reduced pressure. Water was added to the residue, the pH was adjusted to pH 8 and the aqueous layer was extracted with EtOAc. The organic layer was then dried (Na₂SO₄), filtered and then concentrated to give 3,3-dimethyl-2,3-dihydro-1H-indole (16.0 g, 94%). ¹H NMR (400 MHz, CHLOROFORM-d) δ ppm 1.30 (s, 6H) 3.30 (s, 2H) 6.62-6.66 (m, 1H) 6.71-6.76 (m, 1H) 7.02 (s, 2H)

### Step 4. 3,3-dimethyl-2,3-dihydro-1H-indole-6-sulfonic acid

A mixture of 3,3-dimethyl-2,3-dihydro-1H-indole (16.0 g, 109 mmol) in fuming sulphuric acid (60 mL) was stirred at rt for 45 min. The reaction was then heated to 135° C. for 1 h. After cooling the solution was poured into ice water, cooled to −50° C. and allowed to stand for 2 h. The resultant precipitate was collected by filtration to give 3,3-dimethyl-2,3-dihydro-1H-indole-6-sulfonic acid (7 g, 28%). MS (m/z) 228.0 (M+H)⁺. ¹H NMR (400 MHz, DMSO-d₆) δ ppm 1.31 (s, 6H) 3.52 (s, 2H) 7.40 (d, J=7.94 Hz, 1H) 7.58 (s, 1H) 7.64 (dd, J=7.83, 1.43 Hz, 1H)

### Step 5. 1-acetyl-3,3-dimethyl-2,3-dihydro-1H-indole-6-sulfonic acid

To a suspension of 3,3-dimethyl-2,3-dihydro-1H-indole-6-sulfonic acid (7.0 g, 30.8 mmol) in AcOH (70 mL) was added acetic anhydride (6.3 g, 61.6 mmol) and pyridine (4.9 g, 61.6 mmol). The mixture was stirred at 80° C. for 1 h. The reaction was concentrated and the residue washed with 10:1 petroleum ether:EtOAc to give 1-acetyl-3,3-dimethyl-2,3-dihydro-1H-indole-6-sulfonic acid (9.0 g, 84%) as a brown solid. ¹H NMR (400 MHz, DMSO-d₆) δ ppm 1.24 (s, 6H) 3.81 (s, 2H) 7.12 (d, J=7.72 Hz, 1H) 7.27 (d, J=6.84 Hz, 1 H) 8.00 (t, J=6.84 Hz, 2H) 8.27 (s, 1H) 8.52 (t, J=7.83 Hz, 1H) 8.88 (d, J=5.07 Hz, 2H)

### Step 6. 1-acetyl-3,3-dimethyl-2,3-dihydro-1H-indole-6-sulfonyl chloride

To a solution of 1-acetyl-3,3-dimethyl-2,3-dihydro-1H-indole-6-sulfonic acid (9.0 g, 25 mmol) in CH₃CN (100 mL) was added POCl₃ (11.5 g, 75 mmol) and the mixture refluxed for 2 h. The mixture was concentrated and EtOAc and water were added. The layers were separated and the aqueous layer was extracted several times with EtOAc. The combined organics were then dried (Na₂SO₄), filtered and the solvent removed under reduced pressure to give 1-acetyl-3,3-dimethyl-2,3-dihydro-1H-indole-6-sulfonyl chloride (5.1 g, 64%) which was used directly in the next step. MS (m/z) 288.1 (M+H)⁺.

### Step 7. 1-acetyl-N,3,3-trimethyl-2,3-dihydro-1H-indole-6-sulfonamide

A solution of 1-acetyl-3,3-dimethyl-2,3-dihydro-1H-indole-6-sulfonyl chloride (5.1 g, 17.8 mmol) in anhydrous dichloromethane (150 mL) was added to a solution of methylamine in ethanol (50 mL, 30%). The mixture was stirred at rt for 30 min. Water was then added to the mixture and the two layers were separated. The aqueous layer was extracted twice with additional dichloromethane. The combined organics were then dried (Na₂SO₄), filtered and the solvent removed under reduced pressure to give 1-acetyl-N,3,3-trimethyl-2,3-dihydro-1H-indole-6-sulfonamide (4.5 g, 89%) as a brown solid. MS (m/z) 283.0 (M+H)⁺.

### Step 8. N,3,3-trimethyl-2,3-dihydro-1H-indole-6-sulfonamide

To a solution of 1-acetyl-N,3,3-trimethyl-2,3-dihydro-1H-indole-6-sulfonamide (4.5 g, 15.9 mmol) in MeOH (45 mL) was added 4M HCl in MeOH solution (45 mL) and the mixture stirred for 15 h at 50° C. The mixture was then concentrated. The residue was diluted with EtOAc and the pH adjusted to pH 8. The two layers were separated and the aqueous layer was extracted twice with additional EtOAc. The combined organics were then dried (Na₂SO₄), filtered and the solvent removed under reduced pressure. The residue was then purified via flash column chromatography (silica gel, 5:1 to 2:petroleum ether:EtOAc) to to give N,3,3-trimethyl-2,3-dihydro-1H-indole-6-sulfonamide (3.5 g, 76%) as a white solid. MS (m/z) 241.1 (M+H)⁺. ¹H NMR (400 MHz, DMSO-d₆) δ ppm 1.21 (s, 6 H) 2.36 (d, J=5.07 Hz, 3H) 3.22 (d, J=1.54 Hz, 2H) 5.93 (s, 1H) 6.80 (d, J=1.76 Hz, 1H) 6.93 (dd, J=7.61, 1.65 Hz, 1H) 7.12 (d, J=7.72 Hz, 1H) 7.16 (d, J=5.07 Hz, 1H)

### Preparation 20

### N-methyl-1H-indole-6-sulfonamide

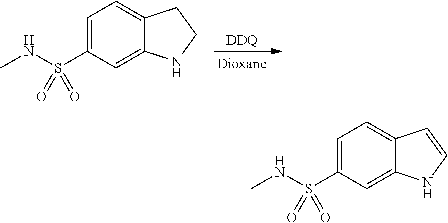

A mixture of N-methyl-2,3-dihydro-1H-indole-6-sulfonamide (500 mg, 2.356 mmol) in 1,4-dioxane (5.889 mL) was treated with DDQ (802 mg, 3.53 mmol) and the reaction stirred for 1 h. The reaction was filtered and the filtrate loaded onto a SCX column (10 g, washed with MeOH followed by 2M ammonia in MeOH). The product eluted in the MeOH wash, and concentration of the appropriate fractions yielded N-methyl-1H-indole-6-sulfonamide (230 mg, crude) as a brown oil which was used as is as an intermediate.

### Preparation 21

### 2-methyl-1,2,3,4-tetrahydro-7-isoquinolinamine

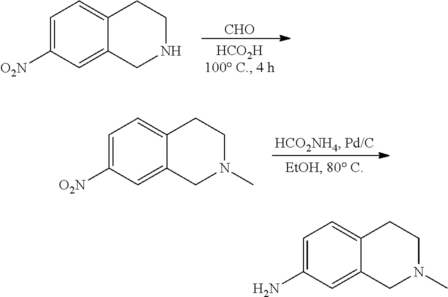

### Step 1. 2-methyl-7-nitro-1,2,3,4-tetrahydroisoquinoline

To a mixture of formaldehyde (26 mL, 944 mmol) and HCO₂H (15 mL), was added 7-nitro-1,2,3,4-tetrahydroisoquinoline (6.32 g, 29.4 mmol). The mixture was heated at 100° C. for 4 h. The reaction was then cooled to rt, poured into ice, and basified to pH 11 with aq. ammonia. The gummy residue which precipitated was extracted with CH₂Cl₂ (2×150 mL). The combined organic extracts were dried over MgSO₄, filtered, and concentrated in vacuo. The compound was loaded onto florisil and purified via flash column chromatography (ISCO, 120 g silica, 0-5% HCl/CH₂Cl₂) to give 2-methyl-7-nitro-1,2,3,4-tetrahydroisoquinoline (5 g, 84%) as an orange solid. ¹H NMR (400 MHz, DMSO-d₆) δ 7.95-8.00 (m, 2H), 7.39 (d, J=8.81 Hz, 1H), 3.58 (s, 2H), 2.93 (t, J=5.79 Hz, 2H), 2.62 (t, J=5.92 Hz, 2H), 2.36 (s, 3H); MS (m/z) 193.1 (M+H)⁺.

### Step 2. 2-methyl-1,2,3,4-tetrahydro-7-isoquinolinamine

To a mixture of 2-methyl-7-nitro-1,2,3,4-tetrahydroisoquinoline (5 g, 26.0 mmol), in ethanol (87 mL), were added 10% Pd/C (2.77 g, 2.60 mmol) and HCO₂.NH₄ (8.20 g, 130 mmol). The resulting mixture was then heated to 80° C. for 3 h. The reaction mixture was then cooled to rt, filtered through Celite®, and concentrated in vacuo to afford 2-methyl-1,2,3,4-tetrahydro-7-isoquinolinamine (3.2 g, 72%) as a tan solid. ¹H NMR (400 MHz, methanol-d₄) δ 6.88 (d, J=8.06 Hz, 1H), 6.58 (dd, J=2.39, 8.18 Hz, 1H), 6.46 (d, J=2.01 Hz, 1H), 3.51 (s, 2H), 2.82 (t, J=5.92 Hz, 2H), 2.70 (t, J=6.04 Hz, 2H), 2.43 (s, 3H); MS (m/z) 163.1 (M+H)⁺.

### Preparation 22

### 6-chloro-N-(3-methylphenyl)-4-pyrimidinamine

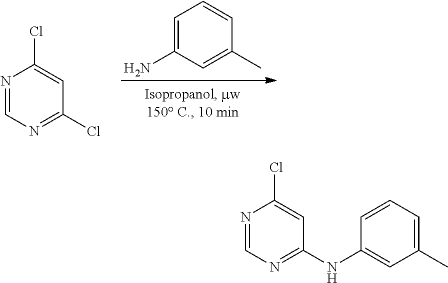

A mixture of dichloropyrimidine (0.556 g, 3.73 mmol) and 3-methyl aniline (0.200 g, 1.866 mmol) in isopropanol (1.678 mL) was heated in a microwave reactor at 150° C. for 10 min. The reaction was concentrated and the residue dissolved in CH₂Cl₂ and purified by silica solid phase extraction (5 g column, washed with CH₂Cl₂ and Et₂O). Concentration of the ethereal fractions yielded 6-chloro-N-(3-methylphenyl)-4-pyrimidinamine (0.264 g, 61%) as a cream solid. ¹H NMR (400 MHz, DMSO-d₆) δ 9.81 (s, 1H), 8.48 (s, 1H), 7.38-7.46 (m, 2H), 7.25 (t, J=7.65 Hz, 1H), 6.92 (d, J=7.28 Hz, 1H), 6.79 (s, 1H), 2.31 (s, 3H); MS (m/z) 220.0 (M+H)⁺.

The following pyrimidinamines were prepared from 4,6-dichloropyrimidine and the aniline indicated using a procedure analogous to that described in Preparation 22:

| Pyrimidinamine | Aniline | MS (m/z) |
|---|---|---|
| 6-chloro-N-(3-chlorophenyl)-4-pyrimidinamine | 3-chloroaniline | 242.0 (M + H)⁺ |
| 6-chloro-N-(4-{[2-(methyloxy)ethyl]oxy}phenyl)- | 4-{[2-(methyloxy)ethyl]oxy}aniline | 280.0 (M + H)⁺ |
| 4-pyrimidinamine | | |
| 6-chloro-N-(3,4-difluorophenyl)-4- | 3,4-difluoroaniline | 241.9 (M + H)⁺ |
| pyrimidinamine | | |
| 3-[(6-chloro-4-pyrimidinyl)amino]-N-methyl-4- | 3-amino-N-methyl-4-(2-methyl-1- | 382.0 (M + H)⁺ |
| (2-methyl-1-pyrrolidinyl)benzenesulfonamide | pyrrolidinyl)benzenesulfonamide | |
| 1-(6-chloro-4-pyrimidinyl)-N-methyl-2,3- | N-methyl-2,3-dihydro-1H-indole- | 325.0 (M + H)⁺ |
| dihydro-1H-indole-6-sulfonamide | 6-sulfonamide | |
| 5-[(6-chloro-4-pyrimidinyl)amino]-2-fluoro-N- | 5-amino-2-fluoro-N-methyl-4- | 415.0 (M + H)⁺ |
| methyl-4-[(2,2,2- | [(2,2,2- | |
| trifluoroethyl)oxy]benzenesulfonamide | trifluoroethyl)oxy]benzenesulfonamide | |
| 1-(6-chloro-4-pyrimidinyl)-N,3,3-trimethyl-2,3- | N,3,3-trimethyl-2,3-dihydro-1H- | 352.9 (M + H)⁺ |
| dihydro-1H-indole-6-sulfonamide | indole-6-sulfonamide | |
| 3-[(6-chloro-4-pyrimidinyl)amino]-N-methyl-4- | 1,1-dimethylethyl [(3-amino-4- | 424.9 (M + H)⁺ |
| [(2,2,2-trifluoro-1,1- | {[2,2,2-trifluoro-1- | |
| dimethylethyl)oxy]benzenesulfonamide | (trifluoromethyl)ethyl]oxy}phenyl)sulfonyl]methylcarbamate | |

### Preparation 23

### 6-chloro-N-(4-chlorophenyl)-4-pyrimidinamine hydrochloride

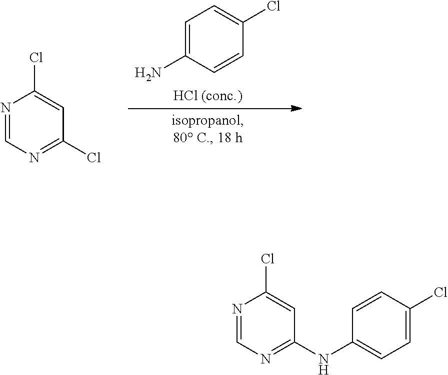

A mixture of 4,6 dichloropyrimidine (0.584 g, 3.92 mmol), 4-chloroaniline (0.250 g, 1.960 mmol) and a few drops of concentrated HCl in isopropanol (4.899 mL) was heated at 80° C. for 18 h. The reaction turned from a clear yellow solution to one containing a white precipitate. This precipitate was collected by filtration to give 6-chloro-N-(4-chlorophenyl)-4-pyrimidinamine hydrochloride (0.443 g, 82%). ¹H NMR (400 MHz, DMSO-d₆) δ 10.33 (s, 1H), 8.50 (s, 1H), 7.69-7.78 (m, J=8.78 Hz, 2H), 7.36-7.43 (m, 2H), 6.93 (s, 1H).

The following pyrimidinamines were prepared from 4,6-dichloropyrimidine and the aniline indicated using procedures analogous to that described in Preparation 23:

| Pyrimidinamine | Aniline | Note | MS (m/z) |
|---|---|---|---|
| 3-[(6-chloro-4- | 3-amino-N-methylbenzene | t-BuOH used as | 299.0 (M + H)⁺ |
| pyrimidinyl)amino]-N- | sulfonamide | solvent, p-TsOH | |
| methylbenzenesulfonamide | | can be | |
| | | substituted for | |
| | | HCl | |
| 6-chloro-N-[4- | 4-(trifluoromethyl)aniline | | 274.0 (M + H)⁺ |
| (trifluoromethyl)phenyl]-4- | | | |
| pyrimidinamine hydrochloride | | | |
| N-(3-bromo-5-methylphenyl)-6- | 3-bromo-5-methylaniline | | 299.9 (M + H)⁺ |
| chloro-4-pyrimidinamine | | | |
| 6-chloro-N-(3-fluorophenyl)-4- | 3-fluoroaniline | | 224.0 (M + H)⁺ |
| pyrimidinamine | | | |
| 6-chloro-N-[4-(1- | [4-(1- | | 248.1 (M + H)⁺ |
| methylethyl)phenyl]-4- | methylethyl)phenyl]amine | | |
| pyrimidinamine | | | |
| 6-chloro-N-[3-chloro-4- | 3-chloro-4- | | 270.1 (M + H)⁺ |
| (methyloxy)phenyl]-4- | (methyloxy)aniline | | |
| pyrimidinamine | | | |
| 6-chloro-N-[4-(2,2,2- | 4-(2,2,2- | | 288.0 (M + H)⁺ |
| trifluoroethyl)phenyl]-4- | trifluoroethyl)aniline | | |
| pyrimidinamine | | | |
| 6-chloro-N-[4-(2,2,2- | 4-[(2,2,2- | | 304.0 (M + H)⁺ |
| trifluoroethyl)phenyl]-4- | trifluoroethyl)oxy]aniline | | |
| pyrimidinamine | | | |
| 6-chloro-N-[4-(1H-pyrazol-1- | 4-(1H-pyrazol-1-yl)aniline | | 272.0 (M + H)⁺ |
| yl)phenyl]-4-pyrimidinamine | | | |
| 3-[(6-chloro-4- | 3-amino-N-methyl-4- | | 345.0 (M + H)⁺ |
| pyrimidinyl)amino]-N-methyl-4- | (methylthio)benzenesulfonamide | | |
| (methylthio)benzenesulfonamide | | | |
| 3-[(6-chloro-4- | 3-amino-N-methyl-4- | | 329.0 (M + H)⁺ |
| pyrimidinyl)amino]-N-methyl-4- | (methyloxy)benzenesulfonamide | | |
| (methyloxy)benzenesulfonamide | | | |
| 3-[(6-chloro-4- | 3-amino-N-methyl-4-[(2,2,2- | | 397.0 (M + H)⁺ |
| pyrimidinyl)amino]-N-methyl-4- | trifluoroethyl)oxy]benzenesulfonamide | | |
| [(2,2,2- | | | |
| trifluoroethyl)oxy]benzenesulfonamide | | | |
| 3-[(6-chloro-4- | 3-amino-4-(ethylthio)-N- | | 359.0 (M + H)⁺ |
| pyrimidinyl)amino]-4-(ethylthio)- | methylbenzenesulfonamide | | |
| N-methylbenzenesulfonamide | | | |
| 3-[(6-chloro-4- | 3-amino-N-methyl-4-[(2- | | 386.7 (M + H)⁺ |
| pyrimidinyl)amino]-N-methyl-4- | methylpropyl)thio]benzenesulfonamide | | |
| [(2- | | | |
| methylpropyl)thio]benzenesulfonamide | | | |
| 3-[(6-chloro-4- | 3-amino-4-[(1,1- | | 387.0 (M + H)⁺ |
| pyrimidinyl)amino]-4-[(1,1- | dimethylethyl)thio]-N- | | |
| dimethylethyl)thio]-N- | methylbenzenesulfonamide | | |
| methylbenzenesulfonamide | | | |
| 3-[(6-chloro-4- | 3-amino-N-methyl-4-[(1- | | 372.9 (M + H)⁺ |
| pyrimidinyl)amino]-N-methyl-4- | methylethyl)thio]benzenesulfonamide | | |
| [(1- | | | |
| methylethyl)thio]benzenesulfonamide | | | |
| 3-[(6-chloro-4- | 3-amino-N-methyl-4-[(2,2,2- | | 413.0 (M + H)⁺ |
| pyrimidinyl)amino]-N-methyl-4- | trifluoroethyl)thio]benzenesulfonamide | | |
| [(2,2,2- | | | |
| trifluoroethyl)thio]benzenesulfonamide | | | |
| 3-[(6-chloro-4- | 3-amino-4-fluoro-N- | | 317.0 (M + H)⁺ |
| pyrimidinyl)amino]-4-fluoro-N- | methylbenzenesulfonamide | | |
| methylbenzenesulfonamide | | | |
| 4-chloro-3-[(6-chloro-4- | 3-amino-4-chloro-N- | | 333.0 (M + H)⁺ |
| pyrimidinyl)amino]-N- | methylbenzenesulfonamide | | |
| methylbenzenesulfonamide | | | |
| 5-[(6-chloro-4- | 5-amino-2-fluoro-N-methyl- | | 428.9 (M + H)⁺ |
| pyrimidinyl)amino]-2-fluoro-N- | 4-[(2,2,2-trifluoro-1- | | |
| methyl-4-[(2,2,2-trifluoro-1- | methylethyl)oxy]benzenesulfonamide | | |
| methylethyl)oxy]benzenesulfonamide | | | |
| 5-[(6-chloro-4- | 5-amino-2-fluoro-N-methyl- | | 363.0 (M + H)⁺ |
| pyrimidinyl)amino]-2-fluoro-N- | 4- | | |
| methyl-4- | (methylthio)benzenesulfonamide | | |
| (methylthio)benzenesulfonamide | | | |

### Preparation 24

### 3-[(6-chloro-4-pyrimidinyl)amino]-4-(dimethylamino)-N-methylbenzenesulfonamide

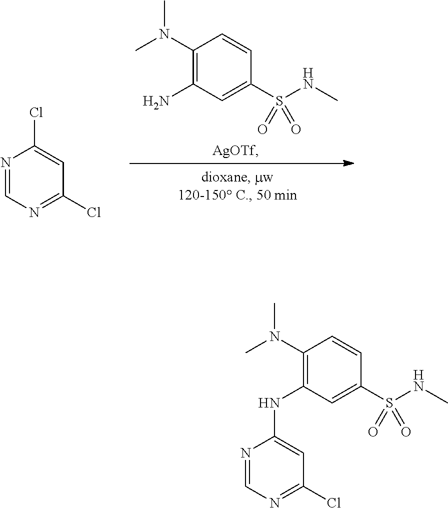

A mixture of 4,6-dichloropyrimidine (0.065 g, 0.436 mmol), 3-amino-4-(dimethylamino)-N-methylbenzenesulfonamide (0.100 g, 0.436 mmol) and AgOTf (0.112 g, 0.436 mmol) in 1,4-dioxane (1.744 mL) was heated in a microwave reactor at 120° C. for 50 min in 10 min intervals. The reaction was filtered through Celite® and the filtrate loaded onto a SCX column (5 g, washed with MeOH and eluted with 2 M ammonia in MeOH). Concentration of the ammonia/MeOH fractions yielded a brown oil which was subsequently loaded onto a silica solid phase extraction column (5 g, eluted with CH₂Cl₂, 50:50 CH₂Cl₂:Et₂O, then Et₂O). Concentration of the appropriate fractions yielded 3-[(6-chloro-4-pyrimidinyl)amino]-4-(dimethylamino)-N-methylbenzenesulfonamide (0.071 g, 48%) as a white solid. ¹H NMR (400 MHz, DMSO-d₆) δ 9.41 (s, 1H), 8.44 (s, 1H), 8.04 (s, 1H), 7.50 (dd, J=2.01, 8.53 Hz, 1H), 7.31 (q, J=4.94 Hz, 1H), 7.22 (d, J=8.53 Hz, 1H), 6.89 (br. s., 1H), 2.73 (s, 6H), 2.42 (d, J=5.02 Hz, 3H); MS (m/z) 341.9 (M+H)⁺.

The following intermediates were prepared from 4,6-dichloropyrimidine and the aniline indicated using procedures analogous to that described in Preparation 24:

| Pyrimidinamine | Aniline | MS (m/z) |
|---|---|---|
| 3-[(6-chloro-4-pyrimidinyl)- | 3-amino-4-(diethylamino)- | 370.1 |
| amino]-4-(diethylamino)-N- | N- | (M + H)⁺ |
| methylbenzenesulfonamide | methylbenzenesulfonamide | |
| 3-[(6-chloro-4-pyrimidinyl)- | 3-amino-4-(2,5-dimethyl-1- | 396.1 |
| amino]-4-(2,5-dimethyl-1- | pyrrolidinyl)-N- | (M + H)⁺ |
| pyrrolidinyl)-N- | methylbenzenesulfonamide | |
| methylbenzenesulfonamide | | |

### Preparation 25

### 4-amino-N-[2-(methyloxy)ethyl]benzamide

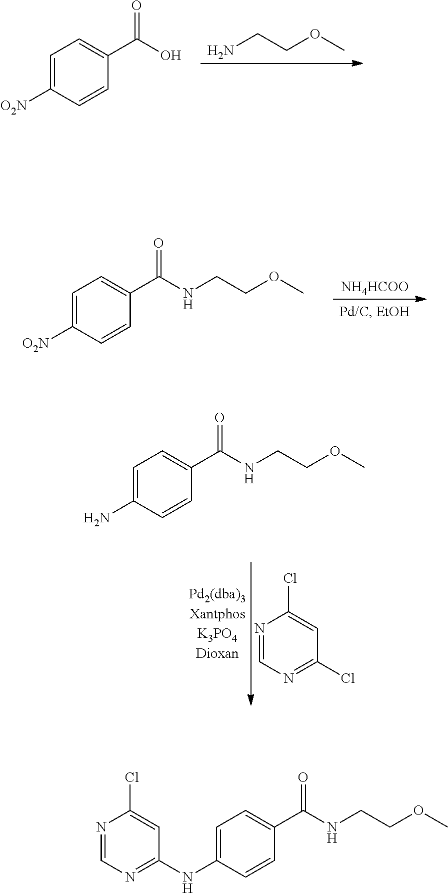

### Step 1: N-[2-(methyloxy)ethyl]-4-nitrobenzamide

A mixture of 4-nitrobenzoic acid (1 g, 5.98 mmol), 2-(methyloxy)ethanamine (618 μl, 7.17 mmol), HOBT (1.833 g, 11.97 mmol), DIPEA (2.090 mL, 11.97 mmol) and EDC (2.294 g, 11.97 mmol) in THF (27.200 mL) was heated to 90° C. for 1 hr. The reaction mixture was concentrated and the residue purified by silica SPE (20 g, eluted with CH₂Cl₂, Et₂O, MeOH). Concentration of the appropriate fractions yielded 1.76 g of a yellow solid which was then partitioned between water and EtOAc. The organic layer was separated and concentrated to give N-[2-(methyloxy)ethyl]-4-nitrobenzamide (1.51 g, crude) which was used as is in the next step.

### Step 2: 4-amino-N-[2-(methyloxy)ethyl]benzamide

A solution of N-[2-(methyloxy)ethyl]-4-nitrobenzamide (1.51 g, 6.73 mmol) in ethanol (33.7 mL) and treated with HCO₂.NH₄ (2.123 g, 33.7 mmol) and Pd/C (0.717 g, 0.673 mmol) then stirred at 40° C. for 2 h. The reaction mixture was filtered through Celite®, and the filtrate concentrated to give ˜1 g of a brown oil which was purified by silica SPE (20 g, eluted with Et₂O, 50:50 Et₂O:EtOAc; EtOAc) to give 4-amino-N-[2-(methyloxy)ethyl]benzamide (791 mg, crude) as a yellow oil which was used as is in the next step.

### Step 3: 4-[(6-chloro-4-pyrimidinyl)amino]-N-[2-(methyloxy)ethyl]benzamide

A mixture of 4-amino-N-[2-(methyloxy)ethyl]benzamide (791 mg, 4.07 mmol), K₃PO₄ (1.729 g, 8.15 mmol), 4,6-dichloropyrimidine (1213 mg, 8.14 mmol), Xantphos (94 mg, 0.163 mmol) and Pd₂(dba)₃ (74.6 mg, 0.081 mmol) in 1,4-dioxane (20.4 mL) was heated at 80° C. under reflux for 24 h. The reaction mixture was then concentrated to give a brown-orange oil, which was then partitioned between CH₂Cl₂/water and separated by hydrophobic frit. The organic layers were concentrated to give ˜1 g orange oil. The residue was then loaded onto a silica SPE (20 g, eluted with CH₂Cl₂, 25:75 Et₂O:CH₂Cl₂, 50:50 CH₂Cl₂:Et₂O, Et₂O and MeOH) to give 4-[(6-chloro-4-pyrimidinyl)amino]-N-[2-(methyloxy)ethyl]benzamide as an orange solid, (433 mg, 35%). MS (m/z) 307.0 (M+H)⁺.

### Preparation 26

### 1-(6-chloro-4-pyrimidinyl)-N-methyl-1H-benzimidazole-6-sulfonamide

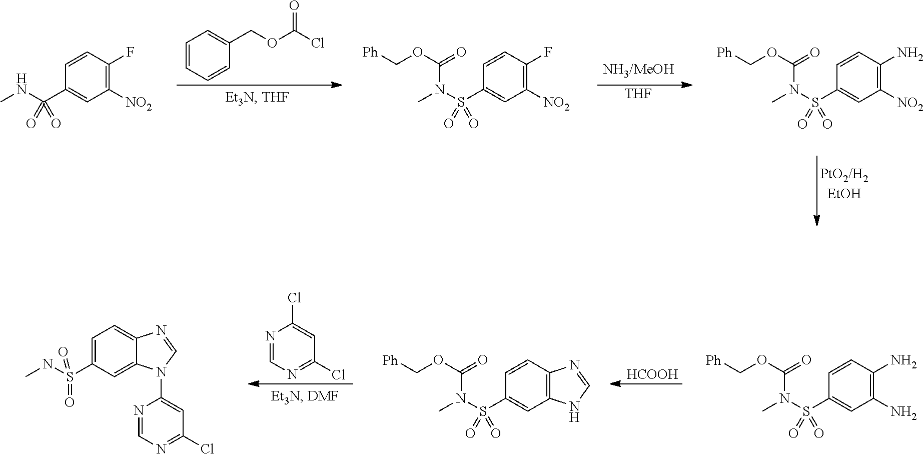

### Step 1: phenylmethyl[(4-fluoro-3-nitrophenyl)sulfonyl]methylcarbamate

A solution of 4-fluoro-N-methyl-3-nitrobenzenesulfonamide (3.0 g, 12.8 mmol) in THF (30 mL) was treated with Et₃N (1.3 g, 12.8 mmol) and then dropwise with phenylmethyl chloridocarbonate (3.27 g, 19.3 mmol) and the mixture stirred at rt for 3 h. The mixture was then concentrated and the residue partitioned between CH₂Cl₂ and water, The organic was then collected and concentrated to give phenylmethyl[(4-fluoro-3-nitrophenyl)sulfonyl]methylcarbamate (3 g, 64%) as a yellow solid. MS (m/z) 391.0 (M+Na)⁺.

### Step 2: phenylmethyl[(4-amino-3-nitrophenyl)sulfonyl]methylcarbamate

A solution of phenylmethyl[(4-fluoro-3-nitrophenyl)sulfonyl]methylcarbamate (3.0 g, 8.5 mmol) in THF (15 mL) was treated with ammonia/MeOH solution (7 M, 5.8 mL) and stirred at rt for 5 h. The reaction mixture was concentrated and the residue (2.8 g, yellow solid) taken on as is into the next step. MS (m/z) 388.1 (M+Na)⁺.

### Step 3: phenylmethyl[(3,4-diaminophenyl)sulfonyl]methylcarbamate

A suspension of phenylmethyl[(4-amino-3-nitrophenyl)sulfonyl]methylcarbamate (2.8 g, 7.7 mmol) and platinum oxide (174 mg, 0.77 mmol) in ethanol (40 mL) was stirred at rt under hydrogen balloon. The mixture was filtered through Celite® and concentrated to give phenylmethyl[(3,4-diaminophenyl)sulfonyl]methylcarbamate (2.7 g, 95%) as a brown oil. MS (m/z) 336.2 (M+H)⁺.

### Step 4: phenylmethyl (1H-benzimidazol-5-ylsulfonyl)methylcarbamate

A solution of phenylmethyl[(3,4-diaminophenyl)sulfonyl]methylcarbamate (2.5 g, 7.46 mmol) in formic acid (20 mL) was heated to 100° C. for 6 h. The reaction was then extracted with CH₂Cl₂. The aqueous layer was adjusted to pH 8 and extracted with CH₂Cl₂. The combined organics were then dried (Na₂SO₄), concentrated and combined with material from a 100 mg trial scale reaction to give phenylmethyl (1H-benzimidazol-5-ylsulfonyl)methylcarbamate (2.1 g, 81%) as a pink solid. MS (m/z) 346.0 (M+H)⁺

### Step 5: 1-(6-chloro-4-pyrimidinyl)-N-methyl-1H-benzimidazole-6-sulfonamide

A solution of phenylmethyl (1H-benzimidazol-5-ylsulfonyl)methylcarbamate (100 mg, 0.290 mmol) and 4,6-dichloropyrimidine (86 mg, 0.579 mmol) in DMF (1367 μl) was treated with Et₃N (81 μl, 0.579 mmol) and heated in the microwave at 150° C. for 90 min. The reaction was diluted by the addition of EtOAc (5 mL) and water (5 mL). The organic layer was separated and concentrated to give a brown oil which was then purified by silica SPE (5 g, eluted with CH₂Cl₂, 50:50 CH₂Cl₂:Et₂O, Et₂O, EtOAc then MeOH). Concentration of the appropriate fractions gave 1-(6-chloro-4-pyrimidinyl)-N-methyl-1H-benzimidazole-6-sulfonamide (40 mg, 1:1 mix of regiosomers) that was used as is in the next step. MS (m/z) 324.0 (M+H)⁺.

### Preparation 27

### 4-amino-N-[2-(methyloxy)ethyl]benzamide

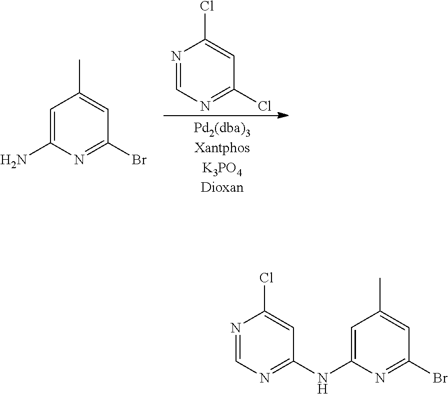

A mixture of 4,6-dichloropyrimidine (476 mg, 3.22 mmol, 6-bromo-4-methyl-2-pyridinamine (300 mg, 1.62 mmol, prepared according to procedures outlined in WO2005061496 and references therein), Pd₂(dba)₃ (28 mg, 0.032 mmol), Xantphos (36 mg, 0.064 mmol) and potassium carbonate (670 mg, 4.89 mmol) in 1,4-dioxane (5 mL) was heated in the microwave at 130° C. for 1 h. The reaction mixture was then poured onto water and the resultant solid collected by filtration and then purified via flash column chromatography (silica gel, 10:1 to 5:1 petroleum Et₂O:EtOAc) to afford 4-amino-N-[2-(methyloxy)ethyl]benzamide (160 mg, 33%) as a white solid, MS (m/z) 300.9 (M+H)⁺.

### Preparation 28

### 6-chloro-N-(3,5-dichloro-2-pyridinyl)-4-pyrimidinamine

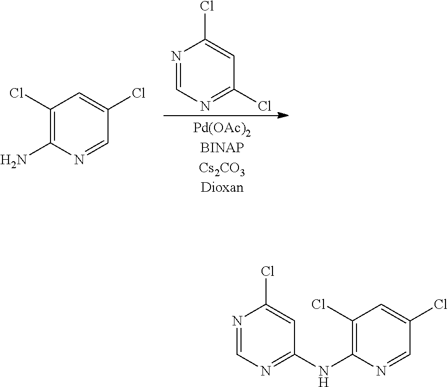

A mixture of 4,6-dichloropyrimidine (823 mg, 5.52 mmol), 3,5-dichloro-2-pyridinamine (450 mg, 2.76 mmol), Cs₂CO₃ (2698 mg, 8.28 mmol), BINAP (68.8 mg, 0.110 mmol) and PdOAc₂ (24.79 mg, 0.110 mmol) was dissolved in 1,4-dioxane (6902 μl) and heated in the microwave at 150° C. for 30 min. The reaction was then concentrated and the residue was then purified by silica SPE (20 g, eluted with 50-50 CH₂Cl₂:hexanes, CH₂Cl₂, 75-25 CH₂Cl₂:Et₂O). Concentration of the appropriate fractions yielded 6-chloro-N-(3,5-dichloro-2-pyridinyl)-4-pyrimidinamine (126 mg, crude) as a yellow solid and a second batch of 6-chloro-N-(3,5-dichloro-2-pyridinyl)-4-pyrimidinamine (310 mg, crude) both batches were used as is in the next step.

The following analog was prepared from the stated pyridinamine and 4,6-dichloropyridine in a procedure analogous to that of Preparation 28:

| Pyrimidinamine | Aniline | MS (m/z) |
|---|---|---|
| N-(5-bromo-6-methyl-2- | 5-bromo-6-methyl-2- | 299.9 (M + H)⁺ |
| pyridinyl)-6-chloro-4- | pyridinamine | |
| pyrimidinamine | | |

### Preparation 29

### 3-[(6-chloro-4-pyrimidinyl)amino]-N-methyl-4-(methylsulfonyl)benzenesulfonamide

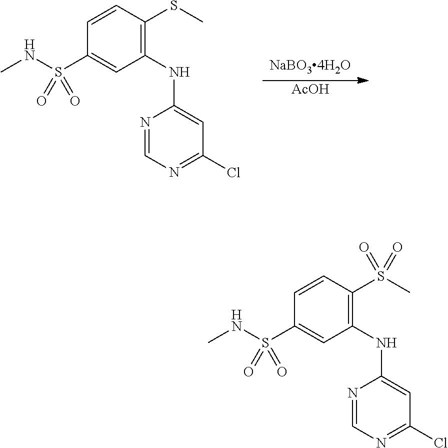

A mixture of 3-[(6-chloro-4-pyrimidinyl)amino]-N-methyl-4-(methylthio) benzenesulfonamide (5.0 g, 14.5 mmol) and sodium perborate tetrahydrate (7.76 g, 43.5 mmol) in AcOH (60 mL) was stirred at 50° C. The mixture was filtered and the filtrate concentrated. The residue was then purified via flash chromatography to give 3-[(6-chloro-4-pyrimidinyl)amino]-N-methyl-4-(methylsulfonyl)benzenesulfonamide (2.1 g, 38%) as a white solid, MS (m/z) 376.9 (M+H)⁺.

The following examples were prepared from the stated sulphide using a procedure analogous to that detailed in Preparation 29:

| Sulphone | Sulphide | MS (m/z) |
|---|---|---|
| 3-[(6-chloro-4-pyrimidinyl)amino]-4- | 3-[(6-chloro-4-pyrimidinyl)amino]-4- | 390.9 (M + H)⁺ |
| (ethylsulfonyl)-N- | (ethylthio)-N- | |
| methylbenzenesulfonamide | methylbenzenesulfonamide | |
| 3-[(6-chloro-4-pyrimidinyl)amino]-N- | 3-[(6-chloro-4-pyrimidinyl)amino]-N- | 404.9 (M + H)⁺ |
| methyl-4-[(1- | methyl-4-[(1- | |
| methylethyl)sulfonyl]benzenesulfonamide | methylethyl)thio]benzenesulfonamide | |
| 3-[(6-chloro-4-pyrimidinyl)amino]-4- | 3-[(6-chloro-4-pyrimidinyl)amino]-4- | 419.1 (M + H)⁺ |
| [(1,1-dimethylethyl)sulfonyl]-N- | [(1,1-dimethylethyl)thio]-N- | |
| methylbenzenesulfonamide | methylbenzenesulfonamide | |

### Example 1

### N-methyl-3-({6-[(3-methylphenyl)amino]-4-pyrimidinyl}amino)benzenesulfonamide trifluoroacetate

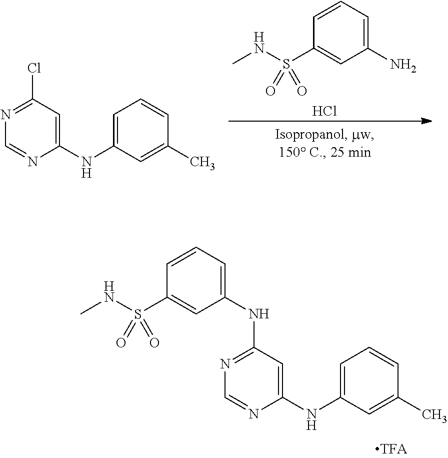

A mixture of 6-chloro-N-(3-methylphenyl)-4-pyrimidinamine (0.264 g, 1.202 mmol), 3-amino-N-methylbenzenesulfonamide (0.224 g, 1.202 mmol) and HCl (0.037 mL, 1.202 mmol) in isopropanol (3.005 mL) was heated in a microwave reactor at 150° C. for 5 min. The reaction mixture was heated for an additional 10 min at 150° C. Additional HCl (0.037 mL, 1.202 mmol) was added and the reaction heated for 10 min in the microwave reactor at 150° C. The reaction was then concentrated and the residue dissolved in CH₂Cl₂ (added a few drops of MeOH to aid solubility) and purified by silica solid phase extraction column (10 g, washed with CH₂Cl₂, Et₂O, EtOAc and acetone). Concentration of the appropriate fractions yielded the crude product. Reverse phase HPLC purification then gave N-methyl-3-({6-[(3-methylphenyl)amino]-4-pyrimidinyl}amino) benzenesulfonamide trifluoroacetate (0.089 g, 15%) as a cream colored solid.

The following compounds were prepared with procedures analogous to that described in Example 1 using the specified pyrimidine in either the free base or HCl salt form and 3-amino-N-methylbenzenesulfonamide:

| Ex. | Name | Structure | Pyrimidine |
|---|---|---|---|
| 2 | 3-({6-[(3-chlorophenyl)amino]- 4-pyrimidinyl}amino)-N- methylbenzenesulfonamide trifluoroacetate | 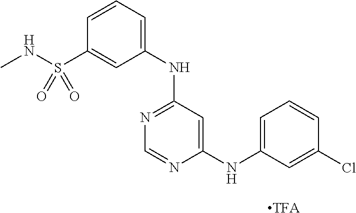 | 6-chloro-N-(3- chlorophenyl)-4- pyrimidineamine |
| 3 | N-methyl-3-{[6-(methylamino)- 4-pyrimidinyl]amino}benzene- sulfonamide hydrochloride | 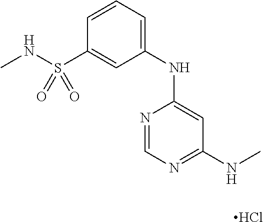 | 6-chloro-N-methyl-4- pyrimidinamine |
| 4 | 3-{[6-(ethylamino)-4- pyrimidinyl]amino}-N- methylbenzenesulfonamide hydrochloride | 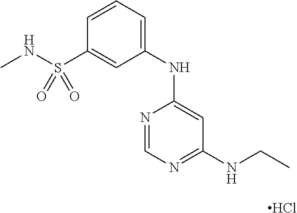 | 6-chloro-N-ethyl-4- pyrimidineamine |
| 5 | 3,3′-(4,6- pyrimidinediyldiimino)bis(N- methylbenzenesulfonamide) trifluoroacetate | 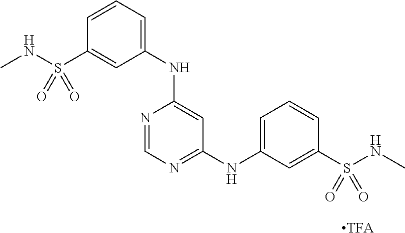 | 4,6-dichloropyrimidine |

The following compounds were prepared with procedures analogous to that described in Example 1 using 6-chloro-N-(4-chlorophenyl)-4-pyrimidinamine in either the free base or HCl salt form and the specified aniline using IPA or NMP as the solvent:

| Ex. | Name | Structure | Aniline |
|---|---|---|---|
| 6 | 3-({6-[(4-chlorophenyl)amino]-4- pyrimidinyl}amino)-5- (dimethylamino)-N- methylbenzenesulfonamide trifluoroacetate | 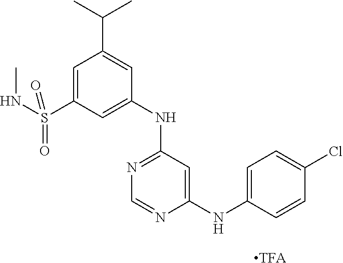 | 3-amino-5- (dimethylamino)-N- methylbenzenesulfona mide |
| 7 | 3-chloro-5-({6-[(4- chlorophenyl)amino]-4- pyrimidinyl}amino)-N- methylbenzenesulfonamide | 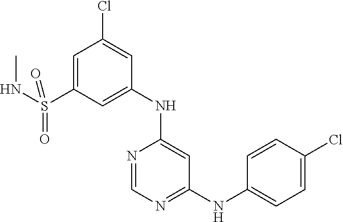 | 3-amino-5-chloro-N- methylbenzenesulfona mide |
| 8 | 3-({6-[(4-chlorophenyl)amino]-4- pyrimidinyl}amino)-N-methyl-4- (propyloxy)benzenesulfonamide trifluoroacetate | 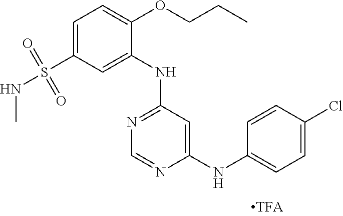 | 3-amino-N-methyl-4- (propyloxy)benzenesul fonamide |
| 9 | 3-({6-[(4-chlorophenyl)amino]-4- pyrimidinyl}amino)-4-(ethyloxy)-N- methylbenzenesulfonamide trifluoroacetate | 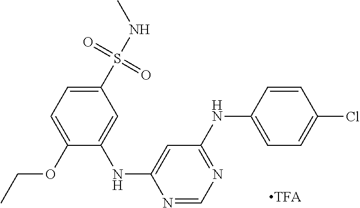 | 3-amino-4-(ethyloxy)- N- methylbenzenesulfona mide |
| 10 | 3-({6-[(4-chlorophenyl)amino]-4- pyrimidinyl}amino)-N-methyl-4-[(2- methylpropyl)oxy]benzenesulfon- amide trifluoroacetate | 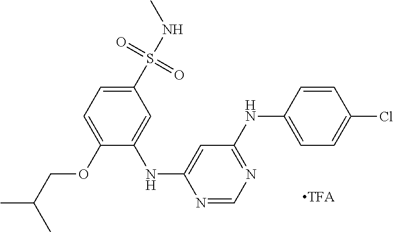 | 3-amino-N-methyl-4- [(2- methylpropyl)oxy]benz enesulfonamide |
| 11 | 3-({6-[(4-chlorophenyl)amino]-4- pyrimidinyl}amino)-4-[(1,2- dimethylpropyl)oxy]-N- methylbenzenesulfonamide trifluoroacetate | 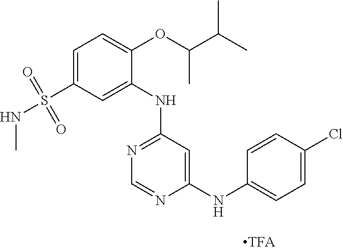 | 3-amino-4-[(1,2- dimethylpropyl)oxy]-N- methylbenzenesulfona mide |
| 12 | 4-chloro-3-({6-[(4- chlorophenyl)amino]-4- pyrimidinyl}amino)-N- methylbenzenesulfonamide trifluoroacetate | 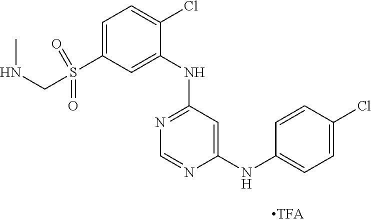 | 3-amino-4-chloro-N- methylbenzenesulfona mide |
| 13 | 3-({6-[(4-chlorophenyl)amino]-4- pyrimidinyl}amino)-N-methyl-4- [(2,2,2-trifluoroethyl)oxy]- benzenesulfonamide trifluoroacetate | 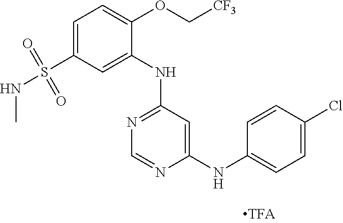 | 3-amino-N-methyl-4- [(2,2,2- trifluoroethyl)oxy]benz enesulfonamide |
| 14 | 3-({6-[(4-chlorophenyl)amino]-4- pyrimidinyl}amino)-4- (cyclohexyloxy)-N- methylbenzenesulfonamide trifluoroacetate | 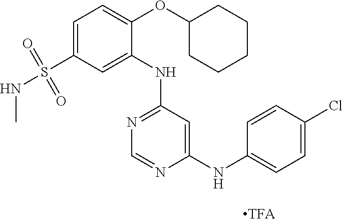 | 3-amino-4- (cyclohexyloxy)-N- methylbenzenesulfona mide |
| 15 | 3-({6-[(4-chlorophenyl)amino]-4- pyrimidinyl}amino)-4-[(1- ethylpropyl)oxy]-N- methylbenzenesulfonamide trifluoroacetate | 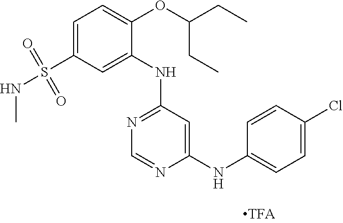 | 3-amino-4-[(1- ethylpropyl)oxy]-N- methylbenzenesulfona mide |
| 16 | 3-({6-[(4-chlorophenyl)amino]-4- pyrimidinyl}amino)-N-methyl-4- [(3,3,3-trifluoropropyl)oxy]- benzenesulfonamide trifluoroacetate | 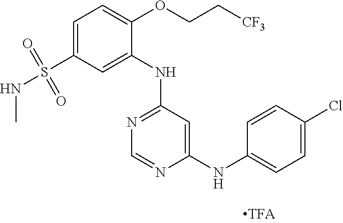 | 3-amino-N-methyl-4- [(3,3,3- trifluoropropyl)oxy]ben zenesulfonamide |
| 17 | 3-({6-[(4-chlorophenyl)amino]-4- pyrimidinyl}amino)-4- (cyclopentyloxy)-N- methylbenzenesulfonamide trifluoroacetate | 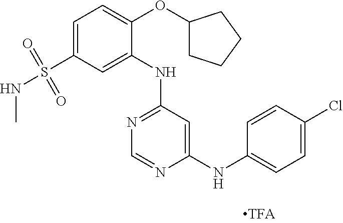 | 3-amino-4- (cyclopentyloxy)-N- methylbenzenesulfona mide |
| 18 | 5-(6-(4- chlorophenylamino)pyrimidin-4- ylamino)-2-fluoro-4-methoxy-N- methylbenzenesulfonamide trifluoroacetate | 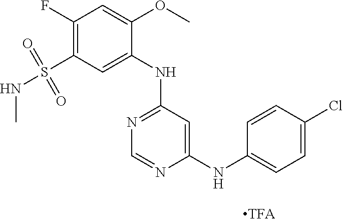 | 5-amino-2-fluoro-N- methyl-4- (methyloxy)benzenesul fonamide |
| 19 | 3-({6-[(4-chlorophenyl)amino]-4- pyrimidinyl}amino)-N-methyl-4- [methyl(2,2,2- trifluoroethyl)amino]benzenesulfona mide trifluoroacetate | 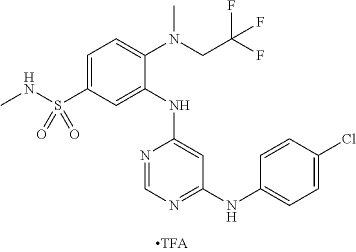 | 3-amino-N-methyl-4- [methyl(2,2,2- trifluoroethyl)amino]ben- zenesulfonamide |
| 20 | 1-{6-[(4-chlorophenyl)amino]-4- pyrimidinyl}-N,3,3-trimethyl-2,3- dihydro-1H-indole-6-sulfonamide trifluoroacetate | 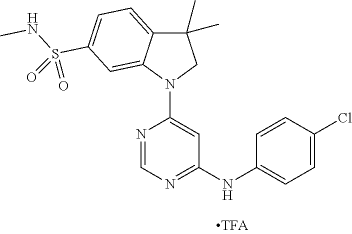 | N,3,3-trimethyl-2,3- dihydro-1H-indole-6- sulfonamide |
| 21 | 3-({6-[(4-chlorophenyl)amino]-4- pyrimidinyl}amino)-N-methyl-4- [(2,2,2-trifluoro-1- methylethyl)oxy]benzenesulfonamide trifluoroacetate | 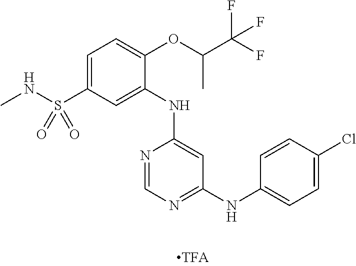 | 3-amino-N-methyl-4- [(2,2,2-trifluoro-1- methylethyl)oxy]benzene- sulfonamide |
| 22 | 5-(6-(4- chlorophenylamino)pyrimidin-4- ylamino)-2-fluoro-N-methyl-4-(2,2,2- trifluoroethoxy)benzenesulfonamide trifluoroacetate | 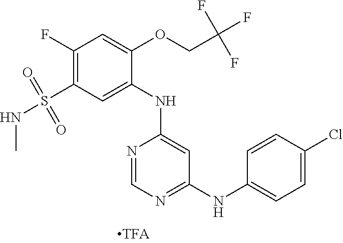 | 5-amino-2-fluoro-N- methyl-4-[(2,2,2- trifluoroethyl)oxy]benzene- sulfonamide |
| 23 | 4-amino-3-({6-[(4- chlorophenyl)amino]-4- pyrimidinyl}amino)-N- methylbenzenesulfonamide trifluoroacetate | 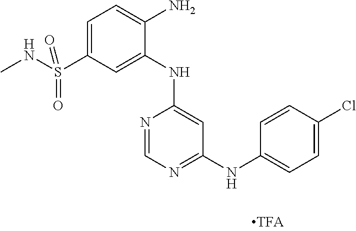 | 3,4-diamino-N- methylbenzenesulfona mide |
| 24 | 5-[6-(4-chloro-phenylamino)- pyrimidin-4-ylamino]-4- dimehtylamino-2-fluoro-N-methyl- benzenesulfonamide | 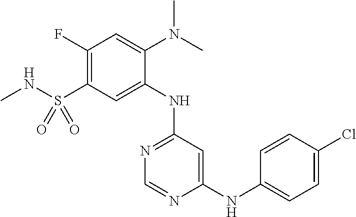 | 5-amino-4- (dimethylamino)-2- fluoro-N- methylbenzenesulfona mide |
| 25 | 3-({6-[(4-chlorophenyl)amino]-4- pyrimidinyl}amino)-4-(3,3-difluoro-1- piperidinyl)-N- methylbenzenesulfonamide trifluoroacetate | 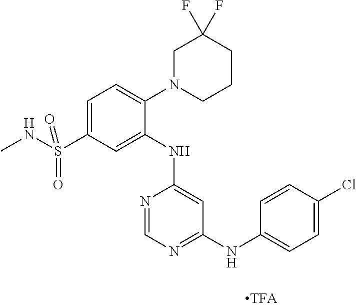 | 3-amino-4-(3,3- difluoro-1-piperidinyl)- N-methylbenzenesulfo Namide |
| 26 | 3-({6-[(4-chlorophenyl)amino]-4- pyrimidinyl}amino)-N-methyl-4- {[2,2,2-trifluoro-1- (trifluoromethyl)ethyl]oxy}benzene- sulfonamide trifluoroacetate | 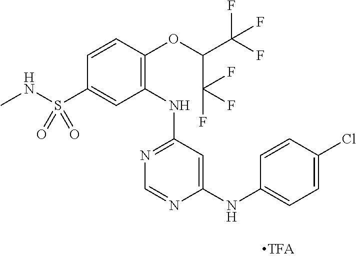 | 1,1-dimethylethyl [(3- amino-4-{[2,2,2- trifluoro-1- (trifluoromethyl)ethyl] oxy}phenyl)sulfonyl] methylcarbamate |

The following compounds were prepared with procedures analogous to that described in Example 1 using 6-chloro-N-(3-fluorophenyl)-4-pyrimidinamine in either the free base or HCl salt form and the specified aniline:

| Ex. | Name | Structure | Aniline |
|---|---|---|---|
| 27 | 4-(dimethylamino)-3-({6-[(3- fluorophenyl)amino]-4- pyrimidinyl}amino)-N- methylbenzenesulfonamide trifluoroacetate | 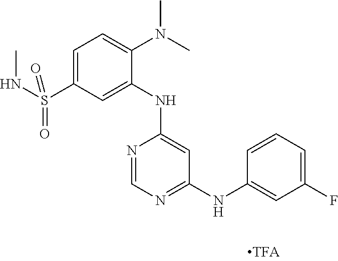 | 3-amino-4- (dimethylamino)-N- methylbenzenesulfona mide |
| 28 | 3-({6-[(3-fluorophenyl)amino]-4- pyrimidinyl}amino)-N-methyl-4- (4-morpholinyl)benzenesulfon- amide trifluoroacetate | 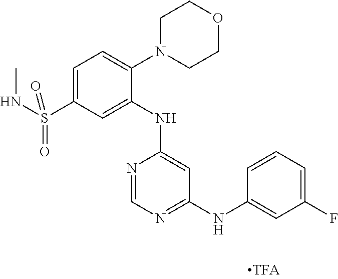 | 3-amino-N-methyl-4- (4- morpholinyl)benzenesul- fonamide |
| 29 | 1-{6-[(3-fluorophenyl)amino]-4- pyrimidinyl}-N-methyl-2,3- dihydro-1H-indole-6-sulfonamide trifluoroacetate | 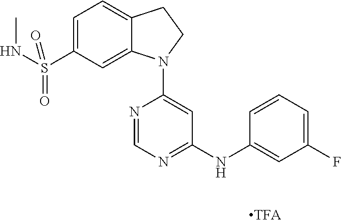 | N-methyl-2,3-dihydro- 1H-indole-6- sulfonamide |
| 30 | 3-({6-[(3-fluorophenyl)amino]-4- pyrimidinyl}amino)-N-methyl-4- (methyloxy)benzenesulfonamide trifluoroacetate | 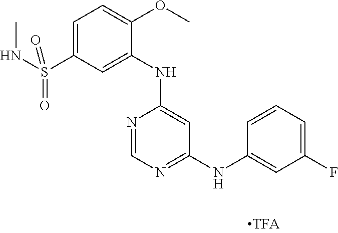 | 3-amino-N-methyl-4- (methyloxy)- benzenesulfonamide |

The following compound was prepared with procedures analogous to that described in Example 1 using 6-chloro-N-[4-(1-methylethyl)phenyl]-4-pyrimidinamine in either the free base or HCl salt form and the specified aniline:

| Ex. | Name | Structure | Aniline |
|---|---|---|---|
| 31 | N-methyl-3-[(6-{[4-(1- methylethyl)phenyl]amino}-4- pyrimidinyl)amino]-4- (methylthio)benzenesulfonamide hydrochloride | 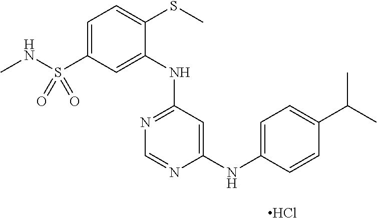 | 3-amino-N-methyl-4- (methylthio)benzene- sulfonamide |

The following compounds were prepared with procedures analogous to that described in Example 1 using 6-chloro-N-[3-chloro-4-(methyloxy)phenyl]-4-pyrimidinamine in either the free base or HCl salt form and the specified aniline:

| Ex. | Name | Structure | Aniline |
|---|---|---|---|
| 32 | 3-[(6-{[3-chloro-4- (methyloxy)phenyl]amino}-4- pyrimidinyl)amino]-N-methyl-4- [(2,2,2- trifluoroethyl)oxy]benzene- sulfonamide hydrochloride | 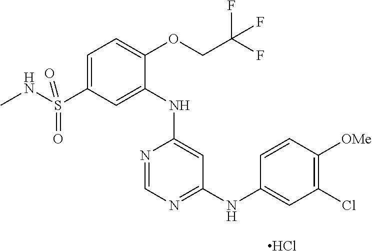 | 3-amino-N-methyl-4- [(2,2,2- trifluoroethyl)oxy] benzenesulfonamide |
| 33 | 3-[(6-{[3-chloro-4- (methyloxy)phenyl]amino}-4- pyrimidinyl)amino]-N-methyl-4- (methyloxy)benzenesulfonamide trifluoroacetate | 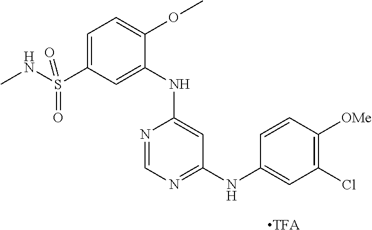 | 3-amino-N-methyl-4- (methyloxy)benzene- sulfonamide |

The following compounds were prepared with procedures analogous to that described in Example 1 using 6-chloro-N-(4-{[2-(methyloxy)ethyl]oxy}phenyl)-4-pyrimidinamine in either the free base or HCl salt form and the specified aniline:

| Ex. | Name | Structure | Aniline |
|---|---|---|---|
| 34 | N-methyl-4-(methyloxy)-3-({6-[(4-{[2- (methyloxy)ethyl]oxy}phenyl)amino]-4- pyrimidinyl}amino)benzenesulfonamide hydrochloride | 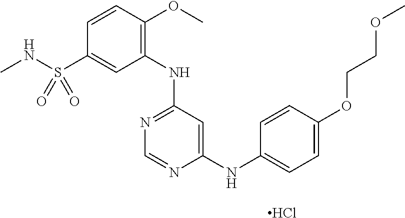 | 3-amino-N- methyl-4- (methyloxy) benzene- sulfonamide |
| 35 | N-methyl-3-({6-[(4-{[2- (methyloxy)ethyl]oxy}phenyl)amino]-4- pyrimidinyl}amino)-4-[(2,2,2- trifluoroethyl)oxy]benzenesulfonamide trifluoroacetate | 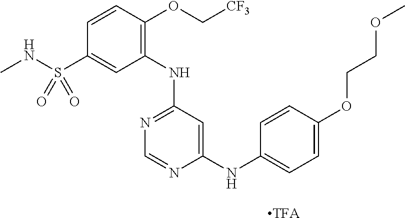 | 3-amino-N- methyl-4-[(2,2,2- trifluoroethyl)oxy] benzene- sulfonamide |

The following compounds were prepared with procedures analogous to that described in Example 1 using 6-chloro-N-[4-(2,2,2-trifluoroethyl)phenyl]-4-pyrimidinamine in either the free base or HCl salt form and the specified aniline:

| Ex. | Name | Structure | Aniline |
|---|---|---|---|
| 36 | N-methyl-4-(methyloxy)-3-[(6-{[4- (2,2,2- trifluoroethyl)phenyl]amino}-4- pyrimidinyl)amino]benzenesulfona mide trifluoroacetate | 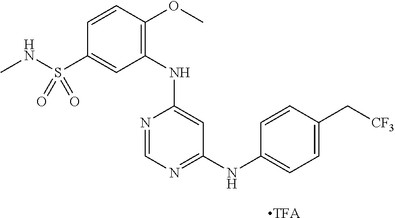 | 3-amino-N-methyl-4- (methyloxy)benzene sulfonamide |
| 37 | N-methyl-4-[(2,2,2- trifluoroethyl)oxy]-3-[(6-{[4-(2,2,2- trifluoroethyl)phenyl]amino}-4- pyrimidinyl)amino]benzenesulfona mide trifluoroacetate | 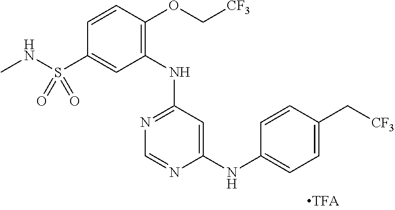 | 3-amino-N-methyl-4- [(2,2,2- trifluoroethyl)oxy]ben zenesulfonamide |
| 38 | N-methyl-3-[(6-{[4-(2,2,2- trifluoroethyl)phenyl]amino}-4- pyrimidinyl)amino]-4-[(2,2,2- trifluoroethyl)thio]benzenesulfona mide trifluoroacetate | 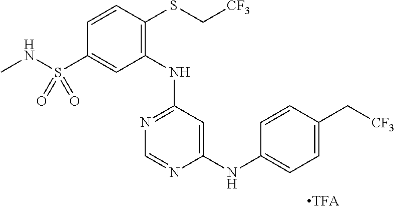 | 3-amino-N-methyl-4- [(2,2,2- trifluoroethyl)thio]ben zenesulfonamide |

The following compound was prepared with procedures analogous to that described in Example 1 using 4-[(6-chloro-4-pyrimidinyl)amino]-N-[2-(methyloxy)ethyl]benzamide in either the free base or HCl salt form and the specified aniline:

| Ex. | Name | Structure | Aniline |
|---|---|---|---|
| 39 | 4-[(6-{[5-[(methylamino)sulfonyl]- 2-(methylthio)phenyl]amino}-4- pyrimidinyl)amino]-N-[2- (methyloxy)ethyl]benzamide trifluoroacetate | 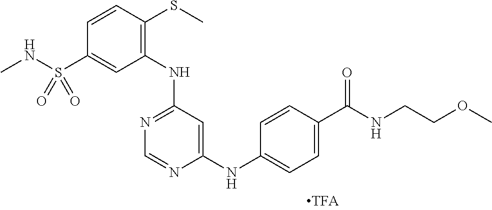 | 3-amino-N- methyl-4- (methylthio)ben zenesulfonamide |

The following compounds were prepared with procedures analogous to that described in Example 1 using 6-chloro-N-[4-(1H-pyrazol-1-yl)phenyl]-4-pyrimidinamine in either the free base or HCl salt form and the specified aniline:

| Ex. | Name | Structure | Aniline |
|---|---|---|---|
| 40 | N-methyl-4-(methyloxy)-3-[(6-{[4- (1H-pyrazol-1-yl)phenyl]amino}- 4- pyrimidinyl)amino]benzenesulfon amide trifluoroacetate | 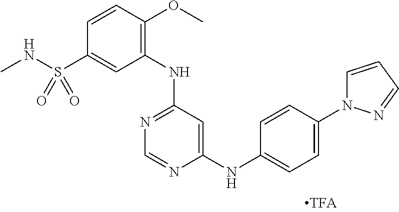 | 3-amino-N-methyl- 4- (methyloxy)benzene sulfonamide |
| 41 | N-methyl-3-[(6-{[4-(1H-pyrazol-1- yl)phenyl]amino}-4- pyrimidinyl)amino]-4-[(2,2,2- trifluoroethyl)oxy]benzenesulfon amide trifluoroacetate | 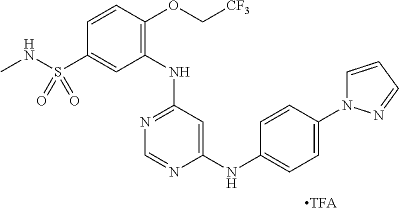 | 3-amino-N-methyl- 4-[(2,2,2- trifluoroethyl)oxy]ben- zenesulfonamide |

The following compound was prepared with procedures analogous to that described in Example 1 using 6-chloro-N-{4-[(2,2,2-trifluoroethyl)oxy]phenyl}-4-pyrimidinamine in either the free base or HCl salt form and the specified aniline:

| Ex. | Name | Structure | Aniline |
|---|---|---|---|
| 42 | N-methyl-4-[(2,2,2- trifluoroethyl)oxy]-3-{[6-({4- [(2,2,2- trifluoroethyl)oxy]phenyl}amino)- 4-pyrimidinyl] amino}benzenesulfon amide trifluoroacetate | 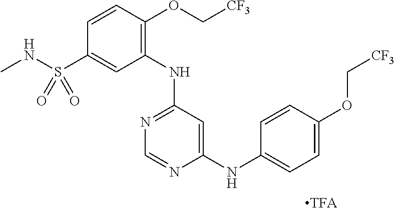 | 3-amino-N-methyl- 4-[(2,2,2- trifluoroethyl)oxy]ben- zenesulfonamide |

The following compounds were prepared with procedures analogous to that described in Example 1 using 6-chloro-N-[4-(trifluoromethyl)phenyl]-4-pyrimidinamine in either the free base or HCl salt form and the specified aniline in NMP as the solvent:

| Ex. | Name | Structure | Aniline |
|---|---|---|---|
| 43 | N-methyl-4-[(2,2,2- trifluoroethyl)oxy]-3-[(6-{[4- (trifluoromethyl)phenyl]amino}-4- pyrimidinyl)amino]benzenesulfon amide trifluoroacetate | 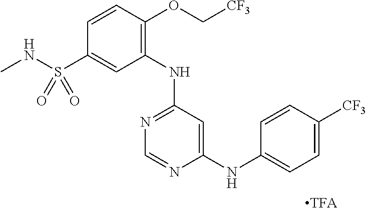 | 3-amino-N-methyl- 4-[(2,2,2- trifluoroethyl)oxy]ben- zenesulfonamide |

The following compounds were prepared with procedures analogous to that described in Example 1 using 6-chloro-N-(3,4-difluorophenyl)-4-pyrimidinamine in either the free base or HCl salt form and the specified aniline using IPA or NMP as the solvent:

| Ex. | Name | Structure | Aniline |
|---|---|---|---|
| 44 | 3-({6-[(3,4- difluorophenyl)amino]-4- pyrimidinyl}amino)-4-fluoro-N- methylbenzenesulfonamide trifluoroacetate | 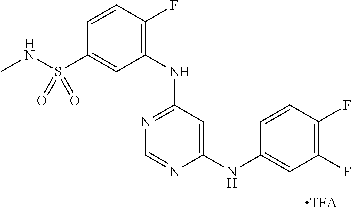 | 3-amino-4-fluoro-N- methylbenzenesulfon- amide |
| 45 | 3-({6-[(3,4- difluorophenyl)amino]-4- pyrimidinyl}amino)-N-methyl-4- [(2,2,2-trifluoro-1- methylethyl)oxy]benzenesulfona mide trifluoroacetate | 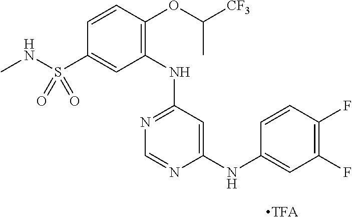 | 3-amino-N-methyl- 4-[(2,2,2-trifluoro-1- methylethyl)oxy]ben zenesulfonamide |
| 46 | 1-{6-[(3,4-difluorophenyl)amino]- 4-pyrimidinyl}-N,3,3-trimethyl- 2,3-dihydro-1H-indole-6- sulfonamide trifluoroacetate | 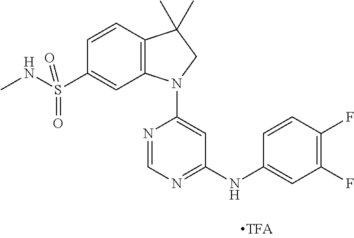 | N,3,3-trimethyl-2,3- dihydro-1H-indole- 6-sulfonamide |

The following compound was prepared with procedures analogous to that described in Example 1 using N-(6-bromo-4-methyl-2-pyridinyl)-6-chloro-4-pyrimidinamine in either the free base or HCl salt form and the specified aniline:

| Ex. | Name | Structure | Aniline |
|---|---|---|---|
| 47 | 3-[6-(6-bromo-4-methyl-pyridin-2- ylamino)-pyrimidin-4-ylamino]-N- methyl-4-(2,2,2-trifluoro-ethoxy)- benzenesulfonamide trifluoroacetate | 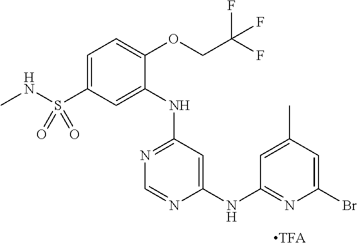 | 3-amino-N-methyl-4- [(2,2,2- trifluoroethyl)oxy]ben- zenesulfonamide |

The following compound was prepared with procedures analogous to that described in Example 1 using 6-chloro-N-(3,5-dichloro-2-pyridinyl)-4-pyrimidinamine in either the free base or HCl salt form and the specified aniline:

| Ex. | Name | Structure | Aniline |
|---|---|---|---|
| 48 | 3-({6-[(3,5-dichloro-2- pyridinyl)amino]-4- pyrimidinyl}amino)-N-methyl-4- [(2,2,2- trifluoroethyl)oxy]benzenesulfona mide trifluoroacetate | 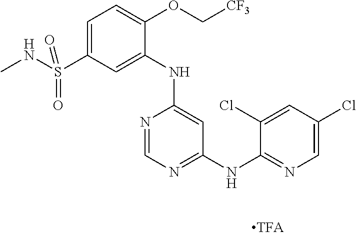 | 3-amino-N-methyl-4- [(2,2,2- trifluoroethyl)oxy]ben- zenesulfonamide |

### Example 49

### 3-{[6-(3-biphenylylamino)-4-pyrimidinyl]amino}-N-methylbenzenesulfonamide trifluoroacetate

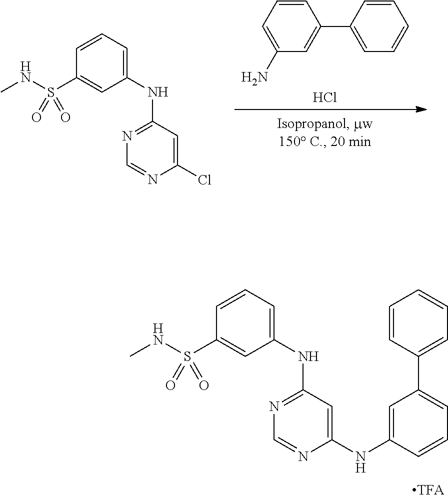

A mixture of 3-[(6-chloro-4-pyrimidinyl)amino]-N-methylbenzenesulfonamide (0.150 g, 0.447 mmol), 3-biphenylamine (0.151 g, 0.895 mmol) and conc. HCl (few drops) in isopropanol (1.119 mL) was heated in a microwave reactor at 150° C. for 20 min. The reaction mixture was concentrated and the residue partitioned between CH₂Cl₂ and water. The organic layer was collected via hydrophobic frit, a precipitate was noted and collected by filtration. This material was dissolved in MeOH/DMSO and purified by reverse phase HPLC (20-65% CH₃CN/H₂O with 0.1% TFA). Concentration of the appropriate fractions yielded 3-{[6-(3-biphenylylamino)-4-pyrimidinyl]amino}-N-methylbenzenesulfonamide trifluoroacetate (0.165 g, 64%) as a white solid.

The following compounds were prepared with procedures analogous to that described in Example 49 using 3-[(6-chloro-4-pyrimidinyl)amino]-N-methylbenzenesulfonamide as either the free base or HCl salt and the specified aniline:

| Ex. | Name | Structure | Aniline |
|---|---|---|---|
| 50 | N-methyl-3-({6-[(4- methylphenyl)amino]-4- pyrimidinyl}amino)benzene sulfonamide hydrochloride | 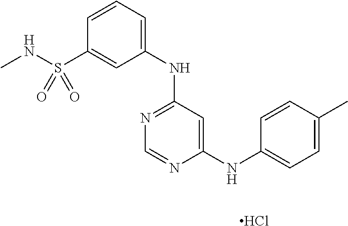 | 4-methylaniline |
| 51 | 3-{[6-({3- [(methylamino)sulfonyl]phenyl} amino)-4- pyrimidinyl]amino}benzamide | 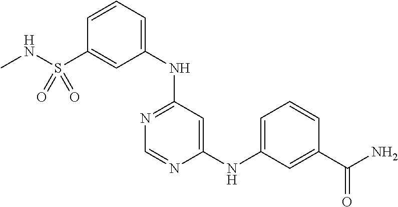 | 3-aminobenzamide |
| 52 | 3-({6-[(3-acetylphenyl)amino]-4- pyrimidinyl}amino)-N- methylbenzenesulfonamide trifluoroacetate | 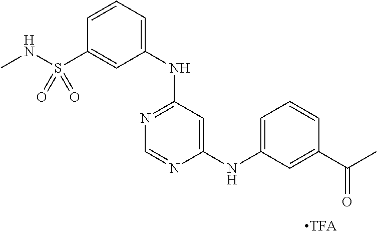 | 1-(3- aminophenyl)ethanone |
| 53 | N-methyl-3-[(6-{[3- (methyloxy)phenyl]amino}-4- pyrimidinyl)amino]benzene sulfonamide trifluoroacetate | 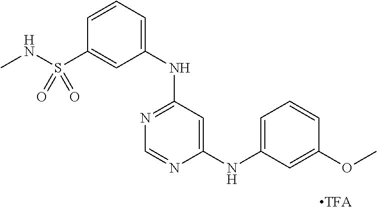 | 3-(methyloxy)aniline |
| 54 | N-(3-{[6-({3- [(methylamino)sulfonyl]phenyl} amino)-4- pyrimidinyl]amino}phenyl)acetamide trifluoroacetate | 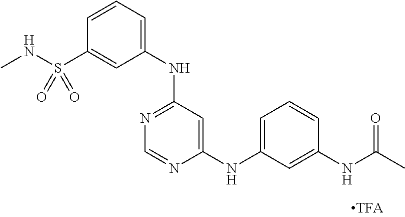 | N-(3- aminophenyl)acetamide |
| 55 | N-methyl-3-{[6-(phenylamino)-4- pyrimidinyl]amino}benzene sulfonamide trifluoroacetate | 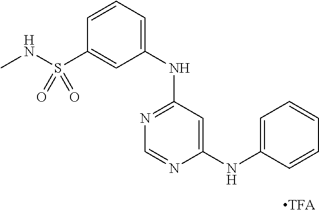 | aniline |
| 56 | 4-{[6-({3- [(methylamino)sulfonyl]phenyl} amino)-4- pyrimidinyl]amino}benzamide trifluoroacetate | 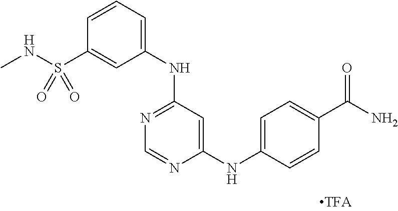 | 4-aminobenzamide |
| 57 | 3-({6-[(4-chlorophenyl)amino]-4- pyrimidinyl}amino)-N- methylbenzenesulfonamide trifluoroacetate | 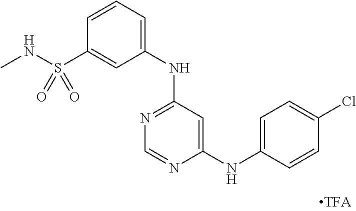 | 4-chloroaniline |
| 58 | N-methyl-3-[(6-{[3- (trifluoromethyl)phenyl]amino}-4- pyrimidinyl)amino]benzene sulfonamide trifluoroacetate | 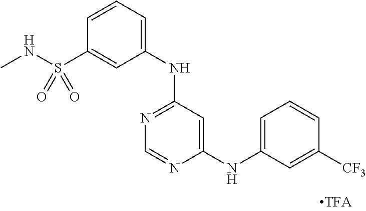 | 3-(trifluoromethyl)aniline |
| 59 | N-methyl-3-({6-[(2-methyl-1,2,3,4- tetrahydro-7-isoquinolinyl)amino]-4- pyrimidinyl}amino)benzene sulfonamide trifluoroaceate | 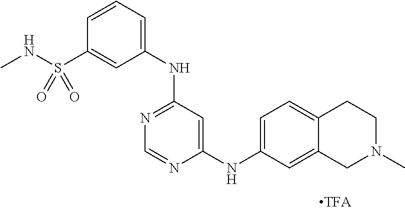 | 2-methyl-1,2,3,4- tetrahydro-7- isoquinolinamine |
| 60 | 3-({6-[(2-fluorophenyl)amino]-4- pyrimidinyl}amino)-N- methylbenzenesulfonamide trifluoroaceate | 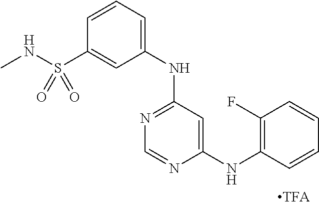 | 2-fluoroaniline |
| 61 | N-methyl-3-[(6-{[3-(4- morpholinylsulfonyl)phenyl]amino}- 4-pyrimidinyl)amino]benzene sulfonamide trifluoroaceate | 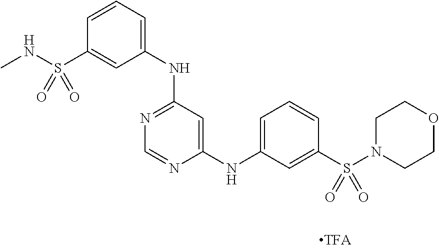 | 3-(4-morpholinylsulfonyl) aniline |
| 62 | 3-{[6-({3- [(ethylamino)sulfonyl]phenyl}amino)- 4-pyrimidinyl]amino}-N- methylbenzenesulfonamide trifluoroaceate | 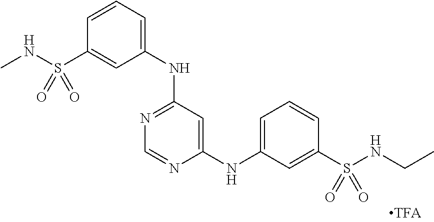 | 3-amino-N- ethylbenzene- sulfonamide |
| 63 | N-methyl-3-[(6-{[3- (methylsulfonyl)phenyl]amino}-4- pyrimidinyl)amino]benzene sulfonamide trifluoroaceate | 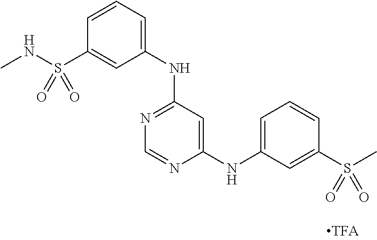 | 3-(methylsulfonyl)aniline |
| 64 | 3-{[6-(1H-indazol-6-ylamino)-4- pyrimidinyl]amino}-N- methylbenzenesulfonamide trifluoroaceate | 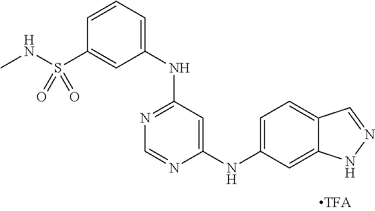 | 1H-indazol-6-amine |
| 65 | 3-{[6-({3- [(methylamino)sulfonyl]phenyl}amino)- 4-pyrimidinyl]amino}-N- phenylbenzamide trifluoroaceate | 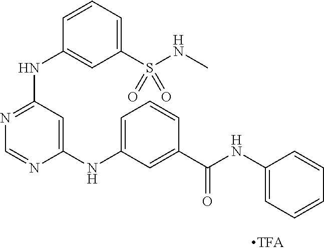 | 3-amino-N- phenylbenzamide |
| 66 | 3-{[6-({3- [(dimethylamino)sulfonyl]phenyl} amino)-4-pyrimidinyl]amino}-N- methylbenzenesulfonamide trifluoroacetate | 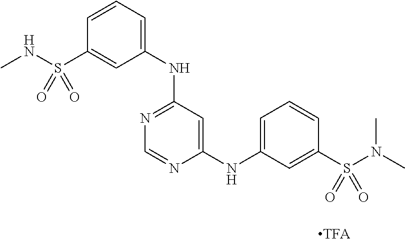 | 3-amino-N,N-dimethyl benzenesulfonamide |
| 67 | 3-[(6-{[3- (aminosulfonyl)phenyl]amino}-4- pyrimidinyl)amino]-N- methylbenzenesulfonamide trifluoroacetate | 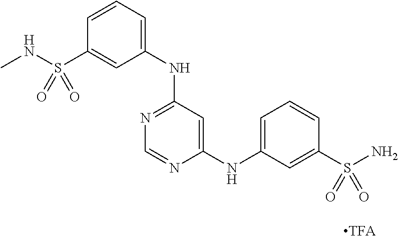 | 3-aminobenzene- sulfonamide |
| 68 | 3-{[6-({3- [(methylamino)sulfonyl]phenyl}amino)- 4-pyrimidinyl]amino}-N-(1- methylethyl)benzenesulfonamide trifluoroacetate | 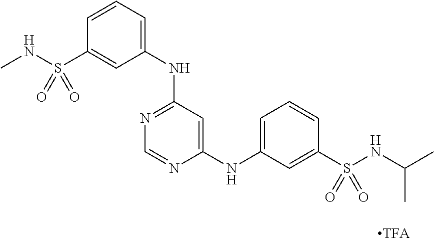 | 3-amino-N-(1- methylethyl)benzene- sulfonamide |
| 69 | 3-({6-[(4-acetylphenyl)amino]-4- pyrimidinyl}amino)-N- methylbenzenesulfonamide trifluoroacetate | 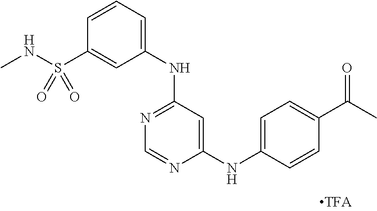 | 1-(4-aminophenyl)- ethanone |
| 70 | N-methyl-3-[(6-{[4- (methylsulfonyl)phenyl]amino}-4- pyrimidinyl)amino]benzene sulfonamide trifluoroacetate | 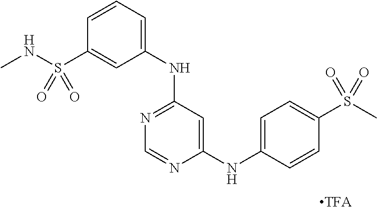 | 4-(methylsulfonyl)aniline |
| 71 | N-(4-{[6-({3- [(methylamino)sulfonyl]phenyl} amino)-4- pyrimidinyl]amino}phenyl)acetamide trifluoroacetate | 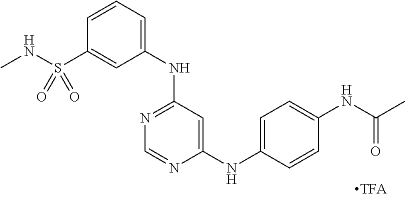 | N-(4-aminophenyl)- acetamide |
| 72 | N-(3-{[6-({3- [(methylamino)sulfonyl]phenyl} amino)-4-pyrimidinyl]amino} phenyl)propanamide trifluoroacetate | 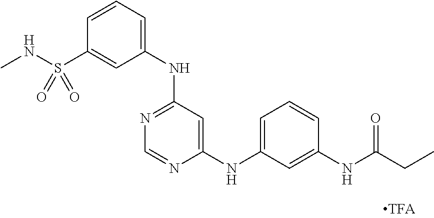 | N-(3-aminophenyl)- propanamide |
| 73 | 4-{[6-({3- [(methylamino)sulfonyl]phenyl} amino)-4-pyrimidinyl]amino}-N- phenylbenzamide trifluoroacetate | 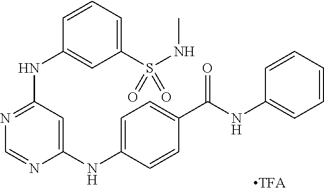 | 4-amino-N- phenylbenzamide |
| 74 | 3-({6-[(1,1-dioxido-2,3-dihydro-1,2- benzisothiazol-6-yl)amino]-4- pyrimidinyl}amino)-N- methylbenzenesulfonamide trifluoroacetate | 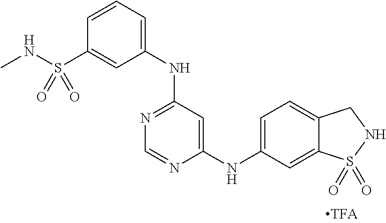 | 2,3-dihydro-1,2- benzisothiazol-6-amine 1,1-dioxide |
| 75 | N-methyl-3-({6-[(2-oxo-2,3-dihydro- 1H-indol-6-yl)amino]-4- pyrimidinyl}amino)benzene sulfonamide trifluoroacetate | 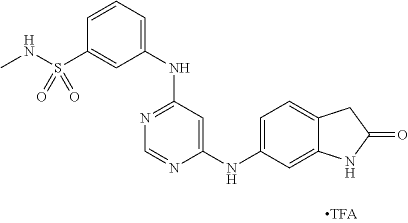 | 6-amino-1,3-dihydro-2H- indol-2-one |
| 76 | N-methyl-3-({6-[(2-methyl-1,3- benzothiazol-5-yl)amino]-4- pyrimidinyl}amino)benzene sulfonamide trifluoroacetate | 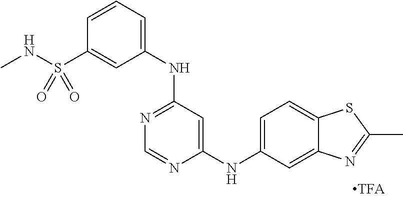 | 2-methyl-1,3- benzothiazol-5-amine |
| 77 | N-methyl-3-({6-[(3- nitrophenyl)amino]-4- pyrimidinyl}amino)benzene sulfonamide trifluoroacetate | 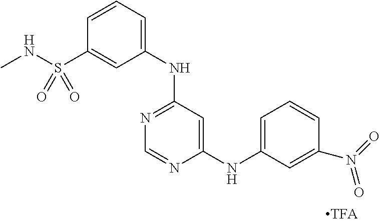 | 3-nitroaniline |
| 78 | N-methyl-3-[(6-{[4-(4- morpholinylcarbonyl)phenyl]amino}- 4-pyrimidinyl)amino]benzene sulfonamide | 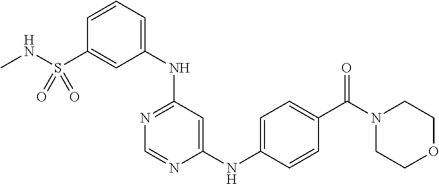 | 4-(4- morpholinylcarbonyl) aniline |
| 79 | N-methyl-4-{[6-({3- [(methylamino)sulfonyl]phenyl}amino)- 4-pyrimidinyl]amino}benzamide trifluoroacetate | 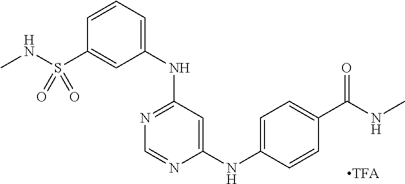 | 4-amino-N- methylbenzamide |
| 80 | 3-{[6-(2,3-dihydro-1,4-benzodioxin- 6-ylamino)-4-pyrimidinyl]amino}-N- methylbenzenesulfonamide trifluoroacetate | 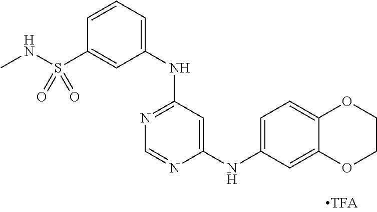 | 2,3-dihydro-1,4- benzodioxin-6-ylamine |
| 81 | N-methyl-3-[(6-{[4- (methyloxy)phenyl]amino}-4- pyrimidinyl)amino]benzene sulfonamide hydrochloride | 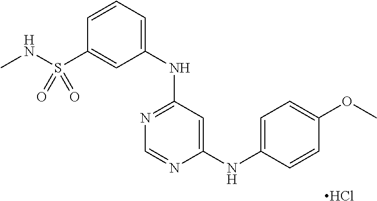 | 4-(methyloxy)aniline |
| 82 | N-methyl-3-[(6-{[4-(4- morpholinyl)phenyl]amino}-4- pyrimidinyl)amino]benzene sulfonamide hydrochloride | 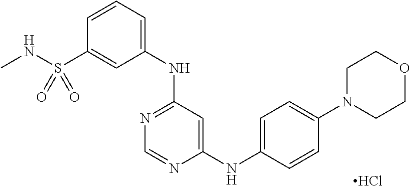 | 4-(4-morpholinyl)aniline |
| 83 | 3-[(6-{[4-(1,1- dimethylethyl)phenyl]amino}-4- pyrimidinyl)amino]-N- methylbenzenesulfonamide trifluoroacetate | 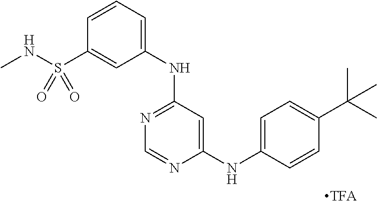 | 4-(1,1- dimethylethyl)aniline |
| 84 | N-methyl-3-[(6-{[3-(4- morpholinyl)phenyl]amino}-4- pyrimidinyl)amino]benzene sulfonamide | 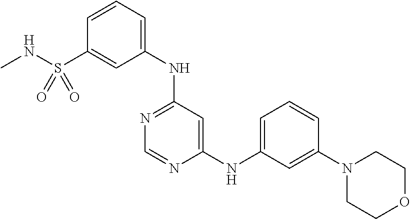 | 3-(4-morpholinyl)aniline |
| 85 | 3-({6-[(3-bromo-5- methylphenyl)amino]-4- pyrimidinyl}amino)-N- methylbenzenesulfonamide hydrochloride | 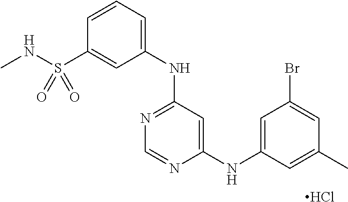 | 3-bromo-5-methylaniline |
| 86 | 3-[(6-{[4- (dimethylamino)phenyl]amino}-4- pyrimidinyl)amino]-N- methylbenzenesulfonamide | 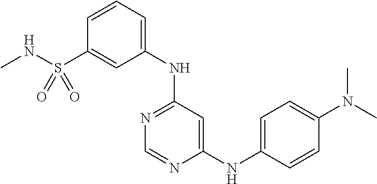 | (4-aminophenyl) dimethylamine |
| 87 | 3-[(6-{[3- (dimethylamino)phenyl]amino}-4- pyrimidinyl)amino]-N- methylbenzenesulfonamide trifluoroacetate | 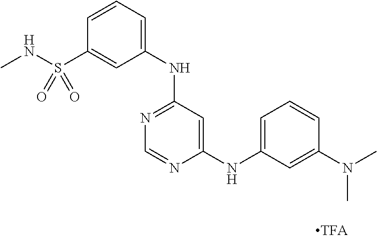 | (3-aminophenyl) dimethylamine |
| 88 | methyl 4-{[6-({3- [(methylamino)sulfonyl]phenyl}amino)- 4-pyrimidinyl]amino}benzoate | 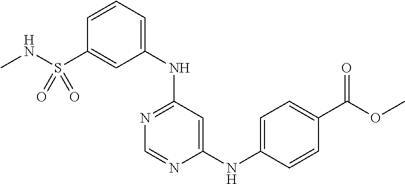 | methyl 4-aminobenzoate |
| 89 | 1-methylethyl 4-{[6-({3- [(methylamino)sulfonyl]phenyl}amino)- 4-pyrimidinyl]amino}benzoate trifluoroacetate | 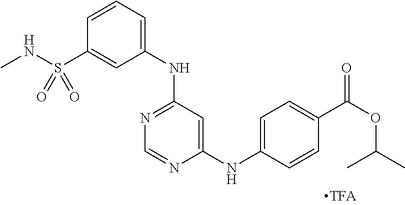 | 1-methylethyl 4- aminobenzoate |
| 90 | 3-({6-[(4-chloro-3- methylphenyl)amino]-4- pyrimidinyl}amino)-N- methylbenzenesulfonamide hydrochloride | 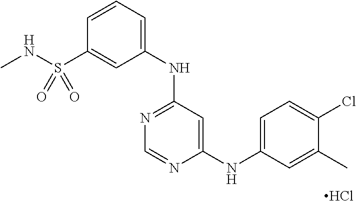 | 4-chloro-3-methylaniline |
| 91 | 3-({6-[(4-fluoro-3- methylphenyl)amino]-4- pyrimidinyl}amino)-N- methylbenzenesulfonamide hydrochloride | 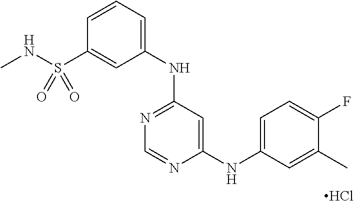 | 4-fluoro-3-methylaniline |
| 92 | 3-{[6-(1H-indol-6-ylamino)-4- pyrimidinyl]amino}-N- methylbenzenesulfonamide | 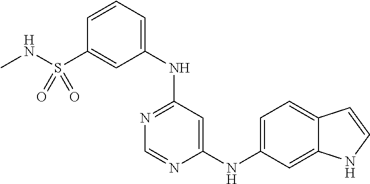 | 1H-indol-6-amine |
| 93 | N-methyl-3-{[6-({3- [(methylsulfonyl)amino]phenyl} amino)-4- pyrimidinyl]amino}benzene sulfonamide | 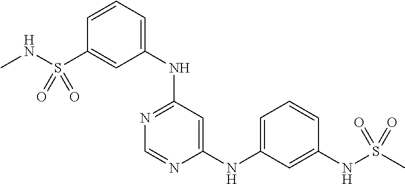 | N-(3-aminophenyl) methanesulfonamide |
| 94 | N-methyl-3-({6-[(3-methyl-1H- indazol-6-yl)amino]-4- pyrimidinyl}amino)benzene sulfonamide | 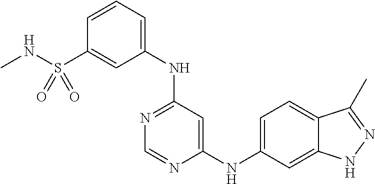 | 3-methyl-1H-indazol-6- amine |
| 95 | 3-({6-[(4-{[2- (diethylamino)ethyl]oxy}phenyl)ami no]-4-pyrimidinyl}amino)-N- methylbenzenesulfonamide | 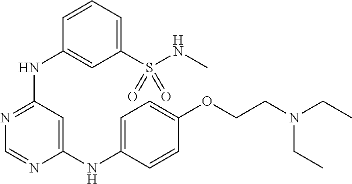 | 4-{[2-(diethylamino) ethyl]oxy}aniline |
| 96 | 1-methylethyl [(3-{[6-({3- [(methylamino)sulfonyl]phenyl}amino)- 4- pyrimidinyl]amino}phenyl)oxy]acetate trifluoroacetate | 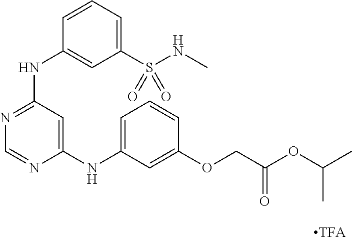 | 1-methylethyl [(3- aminophenyl)oxy]- acetate |
| 97 | 3-{[6-(1,3-benzothiazol-6-ylamino)- 4-pyrimidinyl]amino}-N- methylbenzenesulfonamide trifluoroacetate | 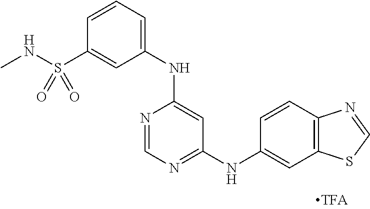 | 1,3-benzothiazol-6- amine |
| 98 | 3-{[6-(1H-indol-5-ylamino)-4- pyrimidinyl]amino}-N- methylbenzenesulfonamide trifluoroacetate | 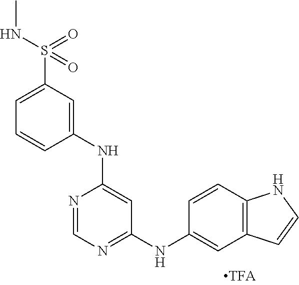 | 1H-indol-5-amine |
| 99 | 3-{[6-(1,3-benzothiazol-5-ylamino)- 4-pyrimidinyl]amino}-N- methylbenzenesulfonamide trifluoroacetate | 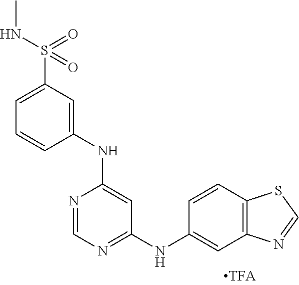 | 1,3-benzothiazol-5- amine |
| 100 | 3-({6-[(3-fluoro-4- methylphenyl)amino]-4- pyrimidinyl}amino)-N- methylbenzenesulfonamide trifluoroacetate | 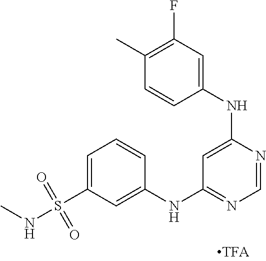 | 3-fluoro-4-methylaniline |
| 101 | 3-({6-[(3-fluorophenyl)amino]-4- pyrimidinyl}amino)-N- methylbenzenesulfonamide trifluoroacetate | 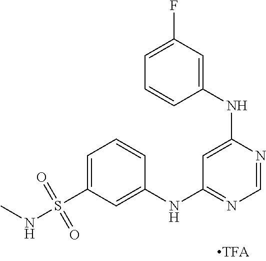 | 3-fluoroaniline |
| 102 | 3-[(6-{[3-fluoro-4- (trifluoromethyl)phenyl]amino}-4- pyrimidinyl)amino]-N- methylbenzenesulfonamide trifluoroacetamide | 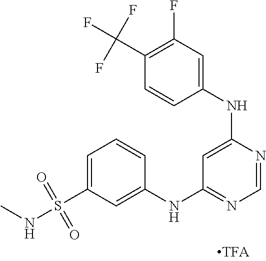 | 3-fluoro-4- (trifluoromethyl)aniline |
| 103 | N-methyl-3-[(6-{[4-(methyloxy)-3- (trifluoromethyl)phenyl]amino}-4- pyrimidinyl)amino]benzenesulfona mide trifluoroacetate | 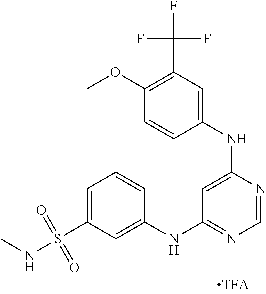 | 4-methoxy-3- (trifluoromethyl)aniline |
| 104 | 3-({6-[(4-chloro-3- fluorophenyl)amino]-4- pyrimidinyl}amino)-N- methylbenzenesulfonamide trifluoroacetate | 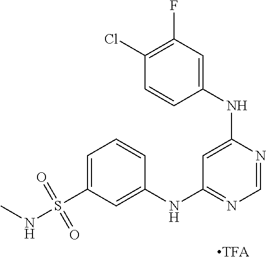 | 4-chloro-3-fluoroaniline |
| 105 | 3-[(6-{[3-fluoro-4- (methyloxy)phenyl]amino}-4- pyrimidinyl)amino]-N- methylbenzenesulfonamide trifluoroacetate | 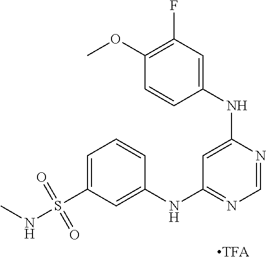 | 3-fluoro-4- methoxyaniline |
| 106 | N-methyl-3-[(6-{[4-methyl-3- (trifluoromethyl)phenyl]amino}-4- pyrimidinyl)amino]benzenesulfona mide trifluoroacetate | 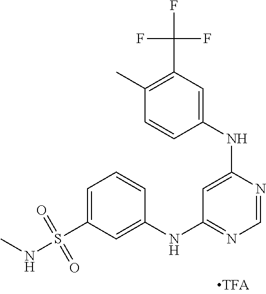 | 4-methyl-3- (trifluoromethyl)aniline |
| 107 | 3-[(6-{[4-chloro-3- (trifluoromethyl)phenyl]amino}-4- pyrimidinyl)amino]-N- methylbenzenesulfonamide trifluoroacetate | 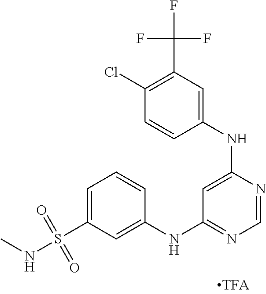 | 4-chloro-3- (trifluoromethyl)aniline |
| 108 | N-methyl-3-[(6-{[4-(2,2,2- trifluoroethyl)phenyl]amino}-4- pyrimidinyl)amino]benzenesulfona mide trifluoroacetate | 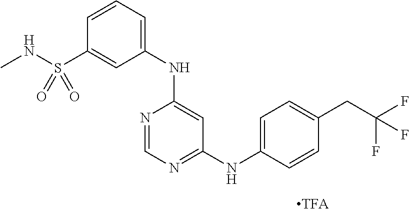 | 4-(2,2,2-trifluoroethyl)- phenylamine |

The following compounds were prepared with procedures analogous to that described in Example 49 using 3-[(6-chloro-4-pyrimidinyl)amino]-N-methyl-4-(methylthio)benzenesulfonamide as either the free base or HCl salt and the specified aniline:

| Ex. | Name | Structure | Aniline |
|---|---|---|---|
| 109 | N-methyl-4-(methylthio)-3-({6-[(2- oxo-1,2,3,4-tetrahydro-7- quinolinyl)amino]-4- pyrimidinyl}amino)benzenesulfona mide | 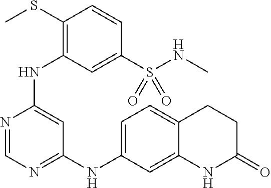 | 7-amino-3,4-dihydro- 2(1H)-quinolinone |
| 110 | 4-[(6-{[5-[(methylamino)sulfonyl]-2- (methylthio)phenyl]amino}-4- pyrimidinyl)amino]benzoic acid trilfuoroacetate | 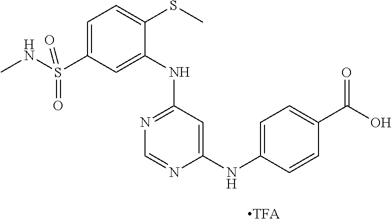 | 4-aminobenzoic acid |

The following compounds were prepared with procedures analogous to that described in Example 49 using 3-[(6-chloro-4-pyrimidinyl)amino]-4-(diethylamino)-N-methylbenzenesulfonamide as either the free base or HCl salt and the specified aniline:

| Ex. | Name | Structure | Aniline |
|---|---|---|---|
| 111 | 3-({6-[(4-chlorophenyl)amino]-4- pyrimidinyl}amino)-4- diethylamino)-N- methylbenzenesulfonamide trifluoroacetate | 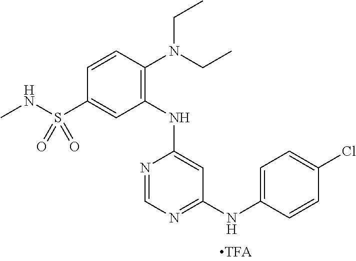 | 4-chloroaniline |

The following compounds were prepared with procedures analogous to that described in Example 49 using 3-[(6-chloro-4-pyrimidinyl)amino]-4-(2,5-dimethyl-1-pyrrolidinyl)-N-methylbenzenesulfonamide as either the free base or HCl salt and the specified aniline:

| Ex. | Name | Structure | Aniline |
|---|---|---|---|
| 112 | 3-({6-[(4-chlorophenyl)amino]-4- pyrimidinyl}amino)-(2,5-dimethyl- 1-pyrrolidinyl)-N- methylbenzenesulfonamide trifluoroacetate | 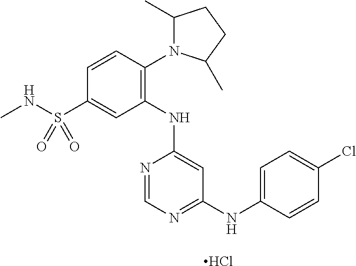 | 4-chloroaniline |

The following compounds were prepared with procedures analogous to that described in Example 49 using 3-[(6-chloro-4-pyrimidinyl)amino]-N-methyl-4-(2-methyl-1-pyrrolidinyl)benzenesulfonamide as either the free base or HCl salt and the specified aniline:

| Ex. | Name | Structure | Aniline |
|---|---|---|---|
| 113 | 3-({6-[(4-chlorophenyl)amino]-4- pyrimidinyl}amino)-N-methyl-4-(2- methyl-1- pyrrolidinyl)benzenesulfonamide trifluoroacetate | 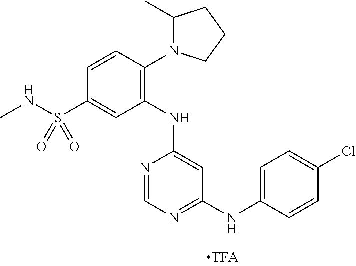 | 4-chloroaniline |

The following compounds were prepared with procedures analogous to that described in Example 49 using 3-[(6-chloro-4-pyrimidinyl)amino]-N,4-dimethylbenzenesulfonamide as either the free base or HCl salt and the specified aniline:

| Ex. | Name | Structure | Aniline |
|---|---|---|---|
| 114 | 3-({6-[(4-chlorophenyl)amino]-4- pyrimidinyl}amino)-N,4- dimethylbenzenesulfonamide trifluoroacetate | 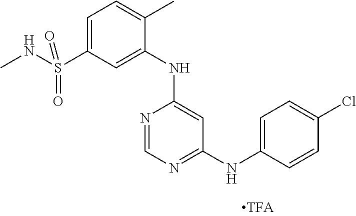 | 4-chloroaniline |

The following compounds were prepared with procedures analogous to that described in Example 49 using 3-[(6-chloro-4-pyrimidinyl)amino]-N-methyl-4-[(2-methylpropyl)thio]benzenesulfonamide as either the free base or HCl salt and the specified aniline:

| Ex. | Name | Structure | Aniline |
|---|---|---|---|
| 115 | 3-(6-(4- chlorophenylamino)pyrimidin-4- ylamino)-4-(isobutylthio)-N- methylbenzenesulfonamide trifluoroacetate | 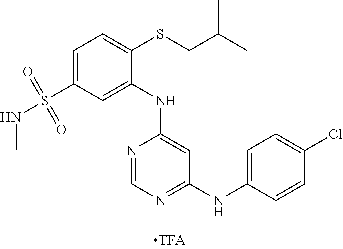 | 4-chloroaniline |
| 116 | 4-(isobutylthio)-N-methyl-3-(6-(4- (trifluoromethyl)phenylamino) pyrimidin-4- ylamino)benzenesulfonamide trifluoroacetate | 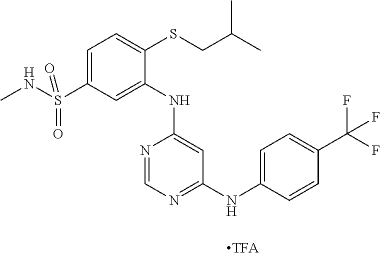 | 4-(trifluoromethyl)aniline |
| 117 | 4-(isobutylthio)-3-(6-(4- isopropylphenylamino)pyrimidin-4- ylamino)-N- methylbenzenesulfonamide trifluoroacetate | 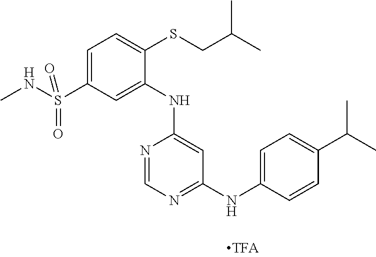 | 4-(1-methylethyl)aniline |

The following compounds were prepared with procedures analogous to that described in Example 49 using 3-[(6-chloro-4-pyrimidinyl)amino]-N-methyl-4-[(2,2,2-trifluoroethyl)oxy]benzenesulfonamide as either the free base or HCl salt and the specified aniline:

| Ex. | Name | Structure | Aniline |
|---|---|---|---|
| 118 | 3-{[6-({4- [(difluoromethyl)oxy]phenyl} amino)-4-pyrimidinyl]amino}- N-methyl- 4-[(2,2,2- trifluoroethyl)oxy] benzenesulfonamide trifluoroacetate | 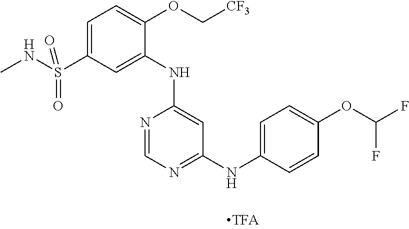 | 4- [(difluoromethyl)oxy] aniline |
| 119 | N-methyl-4-[(2,2,2- trifluoroethyl)oxy]-3-{[6-({4- [(trifluoromethyl)oxy] phenyl}amino)-4- pyrimidinyl] amino}benzenesulfonamide trifluoroacetate | 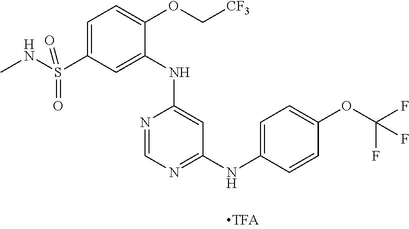 | 4- [(trifluoromethyl)oxy] aniline |
| 120 | 3-({6-[(3,4-difluorophenyl)amino]- 4-pyrimidinyl}amino)-N-methyl-4- [(2,2,2- trifluoroethyl)oxy] benzenesulfonamide hydrochloride | 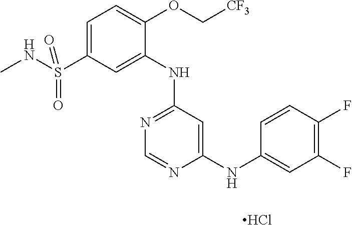 | 3,4-difluoroaniline |
| 121 | 3-({6-[(4-cyanophenyl)amino]-4- pyrimidinyl}amino)-N-methyl-4- [(2,2,2- trifluoroethyl)oxy] benzenesulfonamide trifluoroacetate | 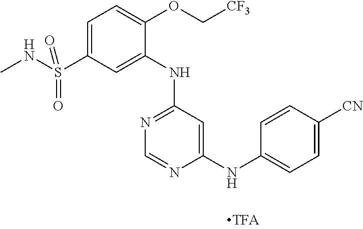 | 4-aminobenzonitrile |

The following compounds were prepared with procedures analogous to that described in Example 49 using 3-[(6-chloro-4-pyrimidinyl)amino]-4-(ethylthio)-N-methylbenzenesulfonamide as either the free base or HCl salt and the specified aniline:

| Ex. | Name | Structure | Aniline |
|---|---|---|---|
| 122 | 3-(6-(4- chlorophenylamino)pyrimidin-4- ylamino)-4-(ethylthio)-N- methylbenzenesulfonamide trifluoroacetate | 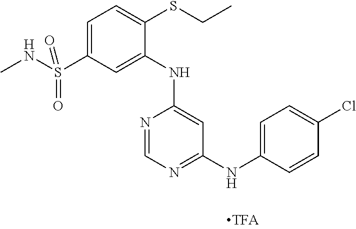 | 4-chloroaniline |
| 123 | 4-(ethylthio)-N-methyl-3-(6-(4- (trifluoromethyl)phenylamino) pyrimidin-4- ylamino)benzenesulfonamide trifluoroacetate | 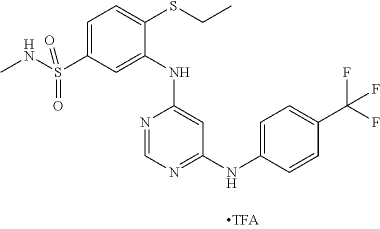 | 4-(trifluoromethyl)aniline |
| 124 | 4-(ethylthio)-3-(6-(4- isopropylphenylamino)pyrimidin-4- ylamino)-N- methylbenzenesulfonamide trifluoroacetate | 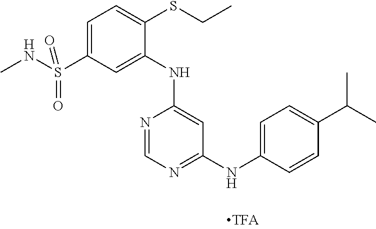 | 4-(1-methylethyl)aniline |

The following compounds were prepared with procedures analogous to that described in Example 49 using 3-[(6-chloro-4-pyrimidinyl)amino]-N-methyl-4-[(2,2,2-trifluoroethyl)thio]benzenesulfonamide as either the free base or HCl salt and the specified aniline:

| Ex. | Name | Structure | Aniline |
|---|---|---|---|
| 125 | 3-(6-(4- chlorophenylamino)pyrimidin-4- ylamino)-N-methyl-4-(2,2,2- trifluoroethylthio) benzenesulfonamide trifluoroacetate | 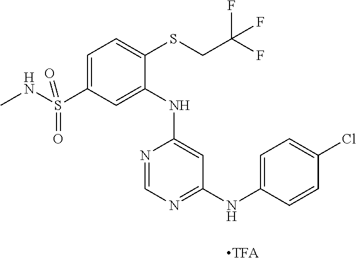 | 4-chloroaniline |
| 126 | N-methyl-4-(2,2,2- trifluoroethylthio)-3-(6-(4- (trifluoromethyl)phenylamino) pyrimidin-4- ylamino)benzenesulfonamide trifluoroacetate | 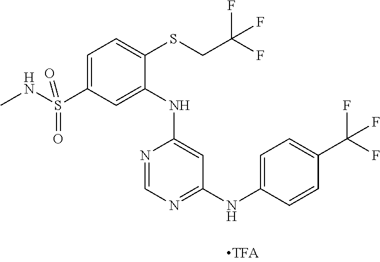 | 4-(trifluoromethyl)aniline |
| 127 | 3-(6-(4- isopropylphenylamino)pyrimidin-4- ylamino)-N-methyl-4-(2,2,2- trifluoroethylthio) benzenesulfonamide trifluoroacetate | 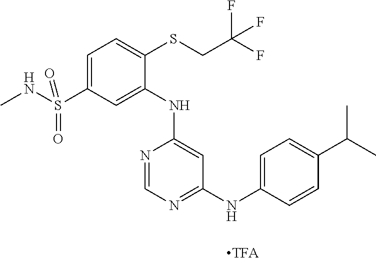 | 4-(1-methylethyl)aniline |

The following compounds were prepared with procedures analogous to that described in Example 49 using 3-[(6-chloro-4-pyrimidinyl)amino]-4-fluoro-N-methylbenzenesulfonamide as either the free base or HCl salt and the specified aniline:

| Ex. | Name | Structure | Aniline |
|---|---|---|---|
| 128 | 4-fluoro-N-methyl-3-{[6-({4- [(trifluoromethyl)oxy]phenyl} amino)-4- pyrimidinyl]amino} benzenesulfonamide trifluoroacetate | 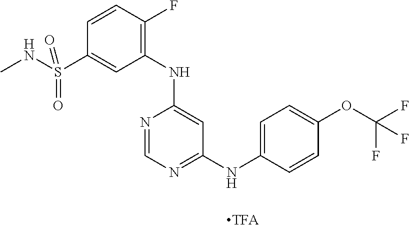 | 4-[(trifluoromethyl)oxy] aniline |
| 129 | 3-{[6-({4- [(difluoromethyl)oxy]phenyl}amino)- 4-pyrimidinyl]amino}-4-fluoro-N- methylbenzenesulfonamide trifluoroacetate | 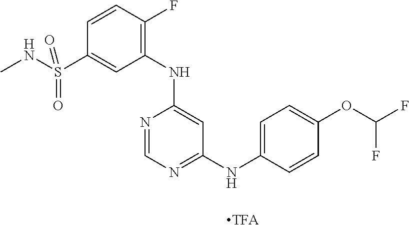 | 4-[(difluoromethyl)oxy] aniline |

The following compounds were prepared with procedures analogous to that described in Example 49 using 4-chloro-3-[(6-chloro-4-pyrimidinyl)amino]-N-methylbenzenesulfonamide as either the free base or HCl salt and the specified aniline:

| Ex. | Name | Structure | Aniline |
|---|---|---|---|
| 130 | 4-chloro-N-methyl-3-[(6-{[4- (trifluoromethyl)pheny]amino}-4- pyrimidinyl)amino] benzenesulfonamide trifluoroacetate | 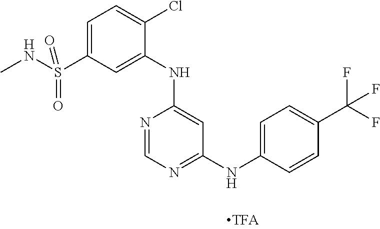 | 4-(trifluoromethyl)aniline |

The following compounds were prepared with procedures analogous to that described in Example 49 using 3-[(6-chloro-4-pyrimidinyl)amino]-N-methyl-4-(methylsulfonyl)benzenesulfonamide as either the free base or HCl salt and the specified aniline:

| Ex. | Name | Structure | Aniline |
|---|---|---|---|
| 131 | 3-({6-[(4-cyanophenyl)amino]-4- pyrimidinyl}amino)-N-methyl-4- (methylsulfonyl) benzenesulfonamide trifluoroacetate | 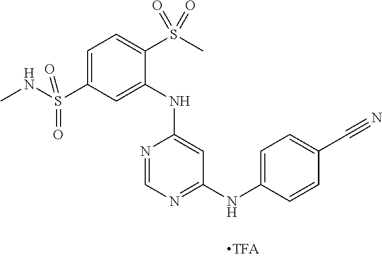 | 4-aminobenzonitrile |
| 132 | 3-({6-[(3,4-difluorophenyl)amino]-4- pyrimidinyl}amino)-N-methyl-4- (methylsulfonyl) benzenesulfonamide trifluoroacetate | 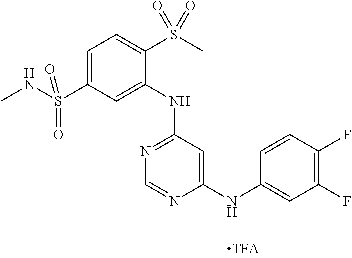 | 3,4-difluoroaniline |
| 133 | 3-(6-(1H-indazol-5- ylamino)pyrimidin-4-ylamino)-N- methyl-4- (methylsulfonyl) benzenesulfonamide | 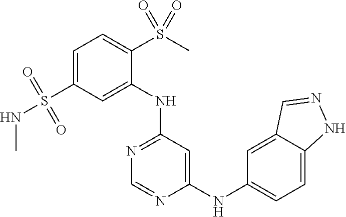 | 1H-indazol-5-amine |
| 134 | 3-(6-(4- (cyanomethyl)phenylamino) pyrimidin-4-ylamino)-N-methyl-4- (methylsulfonyl) benzenesulfonamide | 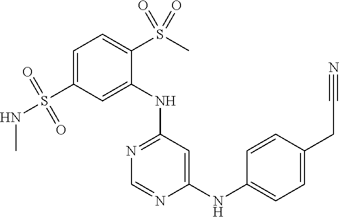 | (4- aminophenyl)acetonitrile |

The following compounds were prepared with procedures analogous to that described in Example 49 using 3-[(6-chloro-4-pyrimidinyl)amino]-4-[(1,1-dimethylethyl)sulfonyl]-N-methylbenzenesulfonamide as either the free base or HCl salt and the specified aniline:

| Ex. | Name | Structure | Aniline |
|---|---|---|---|
| 135 | 4-(tert-butylsulfonyl)-3-(6-(4- chlorophenylamino)pyrimidin-4- ylamino)-N- methylbenzenesulfonamide trifluoroacetate | 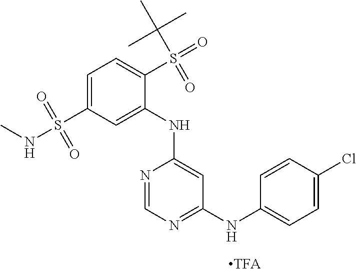 | 4-chloroaniline |

The following compounds were prepared with procedures analogous to that described in Example 49 using 3-[(6-chloro-4-pyrimidinyl)amino]-N-methyl-4-[(2,2,2-trifluoro-1,1-dimethylethyl)oxy]benzenesulfonamide the stated pyrimidine as either the free base or HCl salt and the specified aniline:

| Ex. | Name | Structure | Aniline |
|---|---|---|---|
| 136 | 3-({6-[(4-chlorophenyl)amino]-4- pyrimidinyl}amino)-N-methyl-4- [(2,2,2-trifluoro-1,1- dimethylethyl)oxy] benzenesulfonamide | 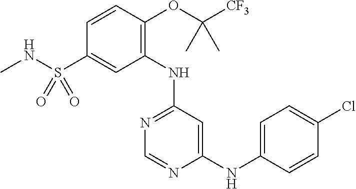 | 4-chloroaniline |

### Example 137

### 3-({6-[(3-bromophenyl)amino]-4-pyrimidinyl}amino)-N-methylbenzenesulfonamide

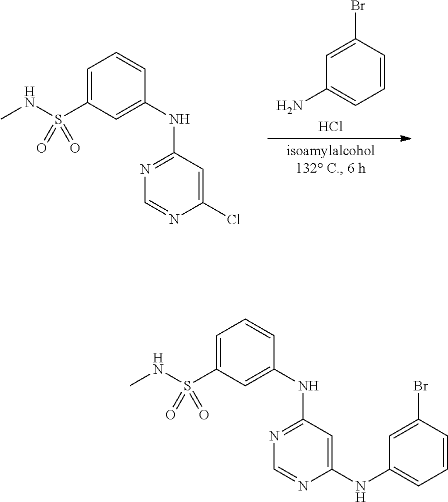

To a solution of 3-({6-[(3-bromophenyl)amino]-4-pyrimidinyl}amino)-N-methylbenzenesulfonamide (15 g, 50 mmol) and 3-bromoaniline (7.8 g, 43 mmol) in isoamylalcohol (10 mL), HCl (3 mL of a 2 M solution, 6 mmol) was added. The resulting mixture was then heated to reflux for 6 h. The mixture was cooled and quenched with NH₄OH and water and stirred for 30 min by which time a precipitate had formed. The precipitate was filtered, washed with hexanes, and dried to give 3-({6-[(3-bromophenyl)amino]-4-pyrimidinyl}amino)-N-methylbenzenesulfonamide (17.5 g, 93%) as a yellow solid.

The following compound was prepared with a procedure analogous to that described in Example 137 using the specified pyrimidine and the appropriate aniline:

| Ex. | Name | Structure | Pyrimidine |
|---|---|---|---|
| 138 | 3-({6-[(3-bromo-4- chlorophenyl)amino]-4- pyrimidinyl}amino)-N- methylbenzenesulfonamide | 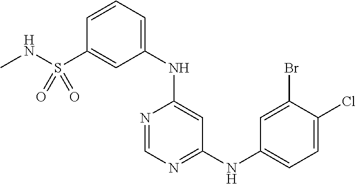 | 3-[(6-chloro-4- pyrimidinyl)amino]-N- methylbenzene sulfonamide |

### Example 139

### 3-[(6-{[3,4-bis(methyloxy)phenyl]amino}-4-pyrimidinyl)amino]-N-methyl-4-(methylthio)benzenesulfonamide trifluoroacetate

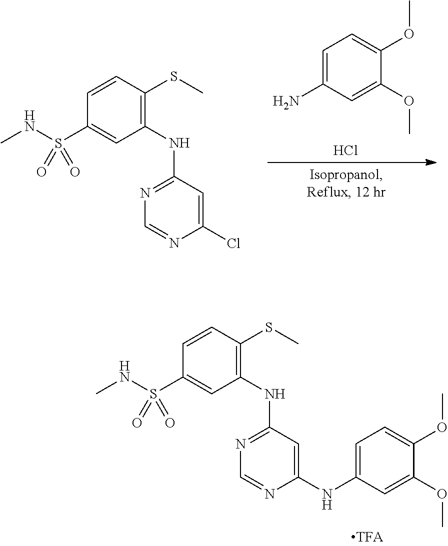

A mixture of 3-[(6-chloro-4-pyrimidinyl)amino]-N-methyl-4-(methylthio) benzenesulfonamide (140 mg, 0.406 mmol) and 3,4-bis(methyloxy)aniline (61 mg, 0.406 mol) in isopropanol (10 mL) and a few drops of conc. HCl were heated at reflux for 12 h. The mixture was then concentrated and purified by preparative HPLC to give 3-[(6-{[3,4-bis(methyloxy)phenyl]amino}-4-pyrimidinyl)amino]-N-methyl-4-(methylthio) benzenesulfonamide trifluoroacetate (38 mg, 46%) as a white solid.

The following compounds were prepared with procedures analogous to that described in Example 139 using 3-[(6-chloro-4-pyrimidinyl)amino]-N-methyl-4-(methylthio) benzenesulfonamide as either the free base or HCl salt and the specified aniline:

| Ex. | Name | Structure | Aniline |
|---|---|---|---|
| 140 | N-methyl-4-methylsulfanyl-3-[6- (3,4,5-trimethoxy-phenylamino)- pyrimidin-4-ylamino]- benzenesulfonamide trifluoroacetate | 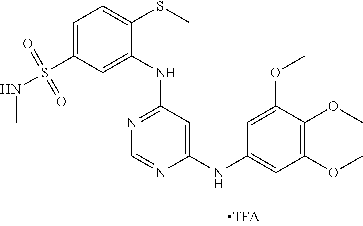 | 3,4,5- tris(methyloxy)aniline |
| 141 | 3-[6-(3,5-dimethoxy-phenylamino)- pyrimidin-4-ylamino]-N-methyl-4- methylsulfanyl- benzenesulfonamide trifluoroacetate | 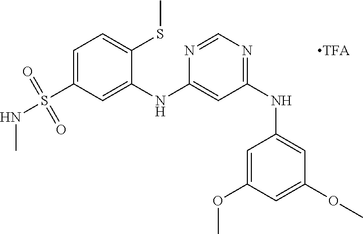 | 3,5- bis(methyloxy)aniline |
| 142 | 3-[6-(4-cyano-phenylamino)- pyrimidin-4-ylamino]-N-methyl-4- methylsulfanyl- benzenesulfonamide trifluoroacetate | 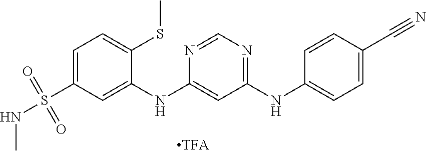 | 4-aminobenzonitrile |
| 143 | 3-[6-(benzo[1,3]dioxol-5-ylamino)- pyrimidin-4-ylamino]-N-methyl-4- methylsulfanyl- benzenesulfonamide trifluoroacetate | 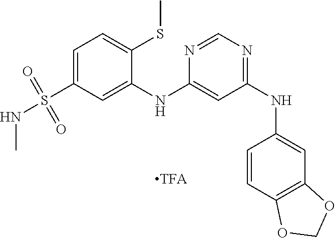 | 1,3-benzodioxol-5- ylamine |
| 144 | 3-[6-(benzothiazol-6-ylamino)- pyrimidin-4-ylamino]-N-methyl-4- methylsulfanyl- benzenesulfonamide trifluoroacetate | 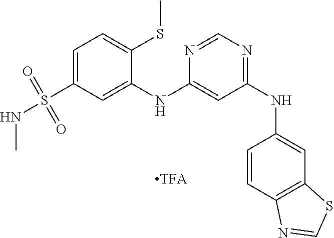 | 1,3-benzothiazol-6- amine |
| 145 | N-methyl-3-[6-(2-methyl- benzothiazol-5-ylamino)-pyrimidin- 4-ylamino]-4-methylsulfanyl- benzenesulfonamide trifluoroacetate | 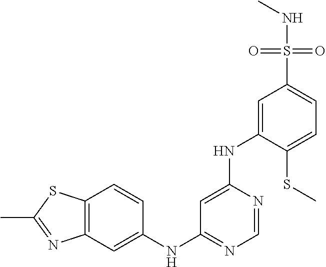 | 2-methyl-1,3- benzothiazol-5-amine |
| 146 | 3-[6-(3-chloro-4-hydroxy- phenylamino)-pyrimidin-4- ylamino]-N-methyl-4- methylsulfanyl- benzenesulfonamide trifluoroacetate | 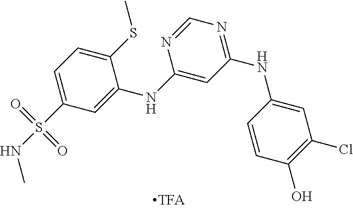 | 4-amino-2-chlorophenol |
| 147 | 3-[6-(3,4-difluoro-phenylamino)- pyrimidin-4-ylamino]-N-methyl-4- methylsulfanyl- benzenesulfonamide trifluoroacetate | 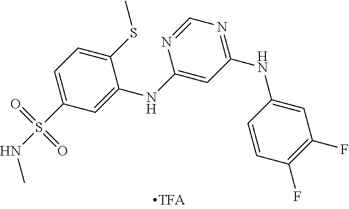 | 3,4-difluoroaniline |
| 148 | N-methyl-4-methylsulfanyl-3-[6-(4- morpholin-4-yl-phenylamino)- pyrimidin-4-ylamino]- benzenesulfonamide di- trifluoroacetate | 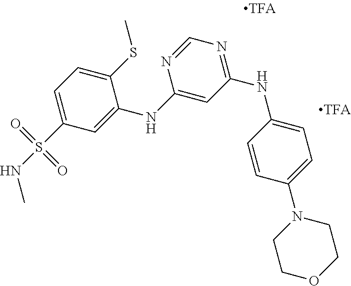 | 4-(4-morpholinyl)aniline |
| 149 | 3-[6-(2,3-dihydro-benzo[1,4]dioxin- 6-ylamino)-pyrimidin-4-ylamino]-N- methyl-4-methylsulfanyl- benzenesulfonamide trifluoroacetate | 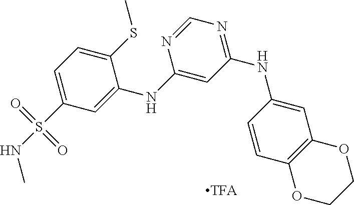 | 2,3-dihydro-1,4- benzodioxin-6-ylamine |
| 150 | N-methyl-4-methylsulfanyl-3-[6-(4- piperidin-1-yl-phenylamino)- pyrimidin-4-ylamino]- benzenesulfonamide trifluoroacetate | 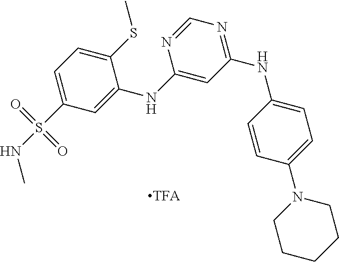 | 4-(1-piperidinyl)aniline |
| 151 | 3-[6-(3-ethynyl-phenylamino)- pyrimidin-4-ylamino]-N-methyl-4- methylsulfanyl- benzenesulfonamide trifluoroacetate | 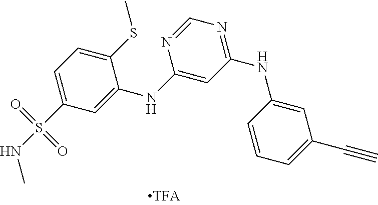 | 3-ethynylaniline |
| 152 | 3-[6-(3,5-dichloro-4-hydroxy- phenylamino)-pyrimidin-4- ylamino]-N-methyl-4- methylsulfanyl- benzenesulfonamide trifluoroacetate | 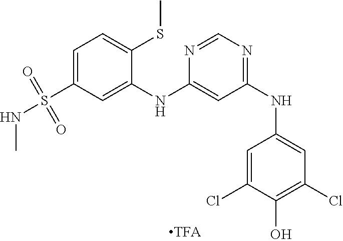 | 4-amino-2,6- dichlorophenol |
| 153 | N-methyl-4-methylsulfanyl-3-{6-[3- (2-methyl-thiazol-4-yl)- phenylamino]-pyrimidin-4- ylamino}-benzenesulfonamide trifluoroacetate | 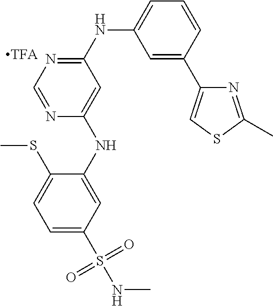 | 3-(2-methyl-1,3-thiazol- 4-yl)aniline |
| 154 | 3-(6-(3-methoxy-5- (trifluoromethyl)phenylamino) pyrimidin-4-ylamino)-N-methyl-4- (methylthio)benzenesulfonamide trifluoroacetate | 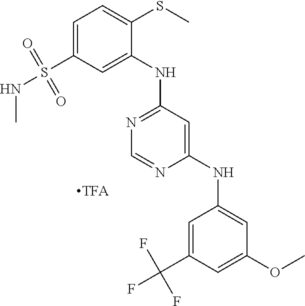 | 3-(methyloxy)-5- (trifluoromethyl)aniline |
| 155 | 3-[6-(1H-indol-5-ylamino)- pyrimidin-4-ylamino]-N-methyl-4- methylsulfanyl- benzenesulfonamide trifluoroacetate | 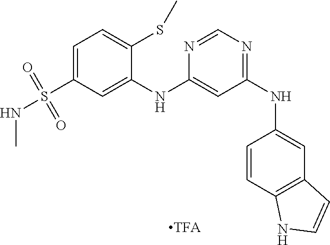 | 1H-indol-5-amine |
| 156 | N-methyl-4-methylsulfanyl-3-[6- (quinolin-6-ylamino)-pyrimidin-4- ylamino]-benzenesulfonamide trifluoroacetate | 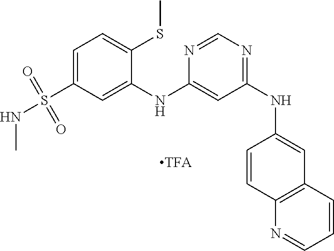 | 6-quinolinamine |
| 157 | 3-[6-(3-chloro-4-cyano- phenylamino)-pyrimidin-4- ylamino]-N-methyl-4- methylsulfanyl- benzenesulfonamide trifluoroacetate | 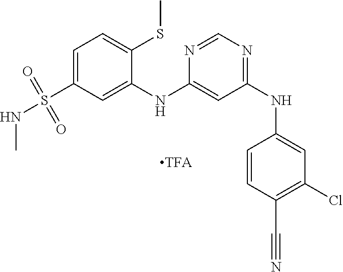 | 4-amino-2- chlorobenzonitrile |
| 158 | N-methyl-4-methylsulfanyl-3-[6-(4- [1,2,4]triazol-4-ylmethyl- phenylamino)-pyrimidin-4- ylamino]-benzenesulfonamide trifluoroacetate | 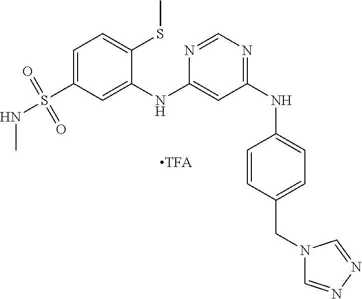 | 4-(4H-1,2,4-triazol-4- ylmethyl)aniline |
| 159 | 3-[6-(1H-indazol-5-ylamino)- pyrimidin-4-ylamino]-N-methyl-4- methylsulfanyl- benzenesulfonamide trifluoroacetate | 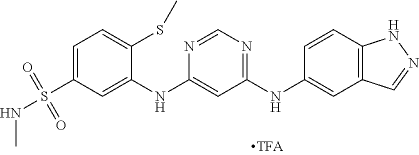 | 1H-indazol-5-amine |
| 160 | 3-[6-(1H-indol-6-ylamino)- pyrimidin-4-ylamino]-N-methyl-4- methylsulfanyl- benzenesulfonamide trifluoroacetate | 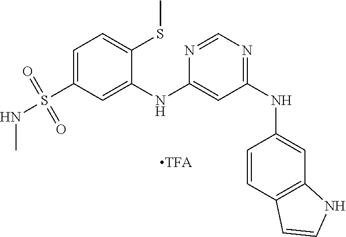 | 1H-indol-6-amine |
| 161 | N-methyl-4-(methylthio)-3-(6-(4- (piperazin-1- yl)phenylamino)pyrimidin-4- ylamino)benzenesulfonamide trifluoroacetate | 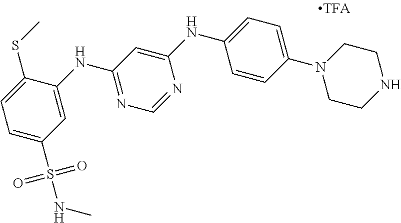 | 4-(1-piperazinyl)aniline |
| 162 | N-methyl-3-(6-(4-methyl-2-oxo-1,2- dihydroquinolin-7- ylamino)pyrimidin-4-ylamino)-4- (methylthio)benzenesulfonamide trifluoroacetate | 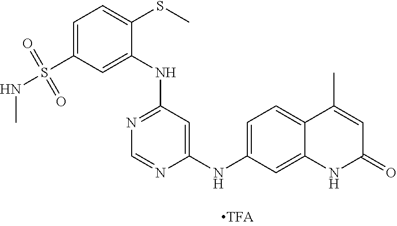 | 7-amino-4-methyl-2(1H)- quinolinone |
| 163 | 3-(6-(1-acetylindolin-6- ylamino)pyrimidin-4-ylamino)-N- methyl-4- (methylthio)benzenesulfonamide trifluoroacetate | 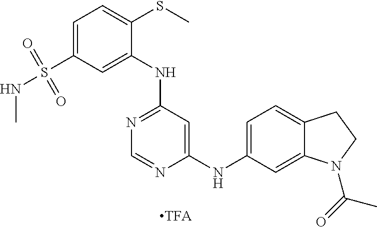 | 1-acetyl-2,3-dihydro-1H- indol-6-amine |
| 164 | N-methyl-3-[6-(2-methyl-4-oxo-4H- chromen-7-ylamino)-pyrimidin-4- ylamino]-4-methylsulfanyl- benzenesulfonamide trifluoroacetate | 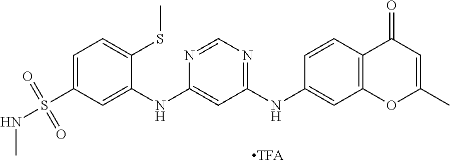 | 7-amino-2-methyl-4H- chromen-4-one |
| 165 | 3-[6-(4-cyanomethyl-phenylamino)- pyrimidin-4-ylamino]-N-methyl-4- methylsulfanyl- benzenesulfonamide trifluoroacetate | 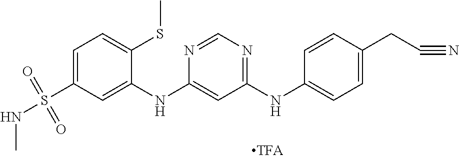 | (4- aminophenyl)acetonitrile |
| 166 | N-methyl-4-methylsulfanyl-3-[6-(5- oxo-5,6,7,8-tetrahydro-naphthalen- 2-ylamino)-pyrimidin-4-ylamino]- benzenesulfonamide trifluoroacetate | 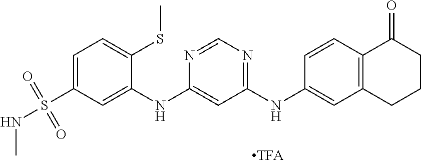 | 6-amino-3,4-dihydro- 1(2H)-naphthalenone |
| 167 | N-methyl-4-methylsulfanyl-3-[6- (3,4,5-trifluoro-phenylamino)- pyrimidin-4-ylamino]- benzenesulfonamide trifluoroacetate | 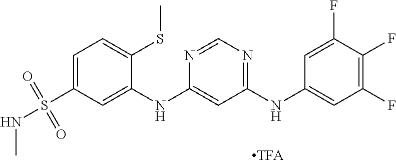 | 3,4,5-trifluoroaniline |
| 168 | N-methyl-3-[6-(4-methyl-2-oxo-2H- chromen-7-ylamino)-pyrimidin-4- ylamino]-4-methylsulfanyl- benzenesulfonamide trifluoroacetate | 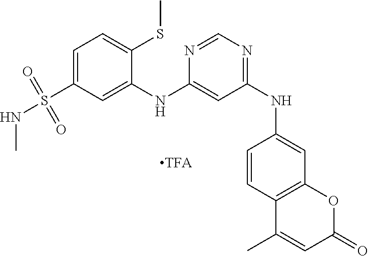 | 7-amino-4-methyl-2H- chromen-2-one |
| 169 | 3-[6-(indan-5-ylamino)-pyrimidin-4- ylamino]-N-methyl-4- methylsulfanyl- benzenesulfonamide trifluoroacetate | 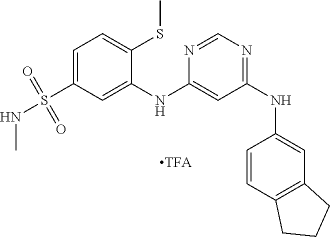 | 2,3-dihydro-1H-inden-5- ylamine |
| 170 | 3-[6-(1H-indazol-6-ylamino)- pyrimidin-4-ylamino]-N-methyl-4- methylsulfanyl- benzenesulfonamide trifluoroacetate | 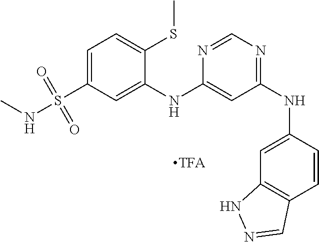 | 1H-indazol-6-amine |
| 171 | N-methyl-3-(6-(2-methyl-1,3- dioxoisoindolin-5- ylamino)pyrimidin-4-ylamino)-4- (methylthio)benzenesulfonamide trifluoroacetate | 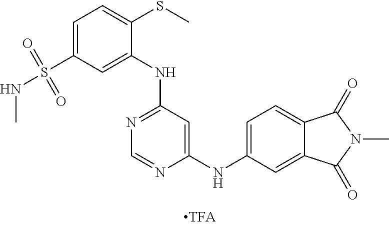 | 5-amino-2-methyl-1H- isoindole-1,3(2H)-dione |

The following compounds were prepared with procedures analogous to that described in Example 139 using 3-[(6-chloro-4-pyrimidinyl)amino]-N-methylbenzenesulfonamide as either the free base or HCl salt and the specified aniline:

| Ex. | Name | Structure | Aniline |
|---|---|---|---|
| 172 | 3-[6-(3,5-dimethoxy-phenylamino)- pyrimidin-4-ylamino]-N-methyl- benzenesulfonamide trifluoroacetate | 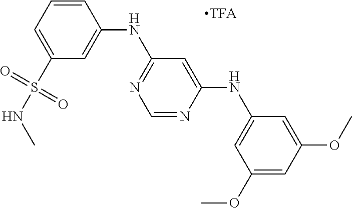 | 3,5- bis(methyloxy)aniline |
| 173 | N-methyl-3-[6-(3,4,5-trimethoxy- phenylamino)-pyrimidin-4- ylamino]-benzenesulfonamide trifluoroacetate | 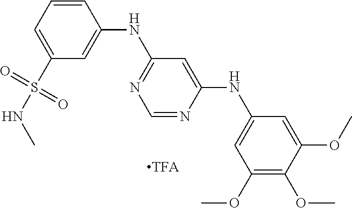 | 3,4,5- tris(methyloxy)aniline |
| 174 | 3-[6-(3-ethynyl-phenylamino)- pyrimidin-4-ylamino]-N-methyl- benzenesulfonamide trifluoroacetate | 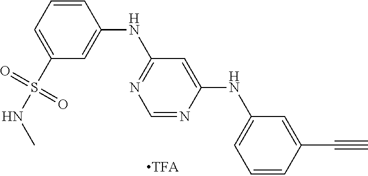 | 3-ethynylaniline |
| 175 | 3-[6-(benzo[1,3]dioxol-5-ylamino)- pyrimidin-4-ylamino]-N-methyl- benzenesulfonamide trifluoroacetate | 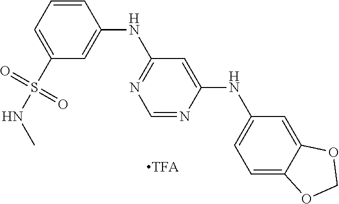 | 1,3-benzodioxol-5- ylamine |
| 176 | 3-[6-(3-chloro-4-hydroxy- phenylamino)-pyrimidin-4- ylamino]-N-methyl- benzenesulfonamide trifluoroacetate | 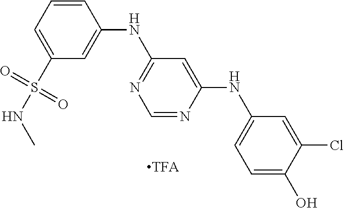 | 4-amino-2-chlorophenol |
| 177 | 3-[6-(3,4-difluoro-phenylamino)- pyrimidin-4-ylamino]-N-methyl- benzenesulfonamide trifluoroacetate | 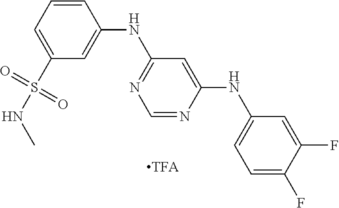 | 3,4-difluoroaniline |
| 178 | N-methyl-3-[6-(4-piperidin-1-yl- phenylamino)-pyrimidin-4- ylamino]-benzenesulfonamide di- trifluoroacetate | 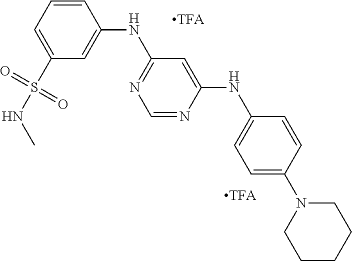 | 4-(1-piperidinyl)aniline |
| 179 | 3-[6-(4-cyano-phenylamino)- pyrimidin-4-ylamino]-N-methyl- benzenesulfonamide trifluoroacetate | 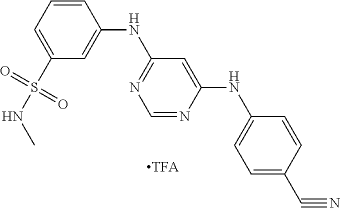 | 4-aminobenzonitrile |
| 180 | N-methyl-3-[6-(2-methyl-4-oxo-4H- chromen-7-ylamino)-pyrimidin-4- ylamino]-benzenesulfonamide trifluoroacetate | 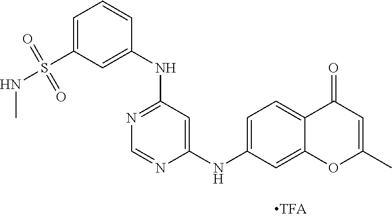 | 7-amino-2-methyl-4H- chromen-4-one |
| 181 | 3-[6-(3,5-dichloro-4-hydroxy- phenylamino)-pyrimidin-4- ylamino]-N-methyl- benzenesulfonamide trifluoroacetate | 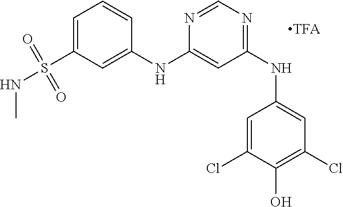 | 4-amino-2,6- dichlorophenol |
| 182 | N-methyl-3-{6-[3-(2-methyl-thiazol- 4-yl)-phenylamino]-pyrimidin-4- ylamino}-benzenesulfonamide trifluoroacetate | 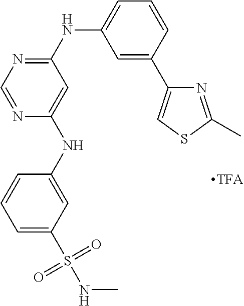 | 3-(2-methyl-1,3-thiazol- 4-yl)aniline |
| 183 | 3-[6-(1H-indazol-5-ylamino)- pyrimidin-4-ylamino]-N-methyl- 1H-indazol-5-amine benzenesulfonamide trifluoroacetate | 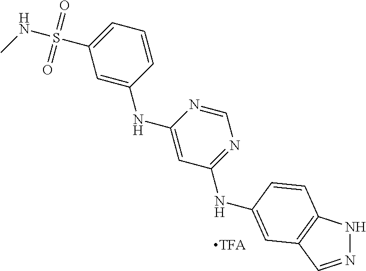 | 1H-indazol-5-amine |
| 184 | N-methyl-3-[6-(5-oxo-5,6,7,8- tetrahydro-naphthalen-2-ylamino)- pyrimidin-4-ylamino]- benzenesulfonamide trifluoroacetate | 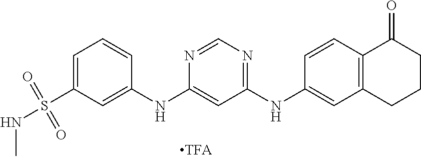 | 6-amino-3,4-dihydro- 1(2H)-naphthalenone |
| 185 | 3-[6-(4-cyanomethyl-phenylamino)- pyrimidin-4-ylamino]-N-methyl- benzenesulfonamide trifluoroacetate | 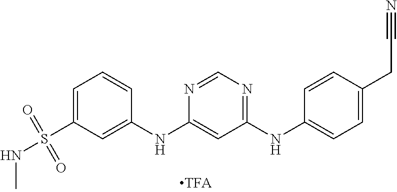 | (4- aminophenyl)acetonitrile |
| 186 | N-methyl-3-[6-(4-methyl-2-oxo-2H- chromen-7-ylamino)-pyrimidin-4- ylamino]-benzenesulfonamide trifluoroacetate | 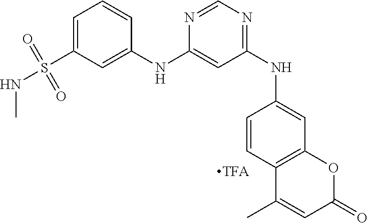 | 7-amino-4-methyl-2H- chromen-2-one |
| 187 | 3-[6-(1-acetyl-2,3-dihydro-1H- indol-6-ylamino)-pyrimidin-4- ylamino]-N-methyl- benzenesulfonamide trifluoroacetate | 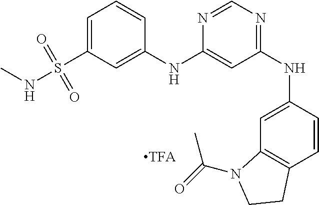 | 1-acetyl-2,3-dihydro-1H- indol-6-amine |
| 188 | 3-[6-(3-methoxy-5-trifluoromethyl- phenylamino)-pyrimidin-4- ylamino]-N-methyl- benzenesulfonamide trifluoroacetate | 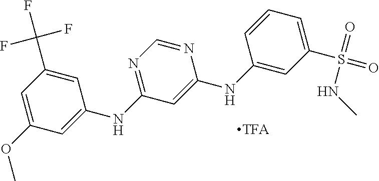 | 3-(methyloxy)-5- (trifluoromethyl)aniline |
| 189 | N-methyl-3-[6-(4-methyl-2-oxo-1,2- dihydro-quinolin-7-ylamino)- pyrimidin-4-ylamino]- benzenesulfonamide trifluoroacetate | 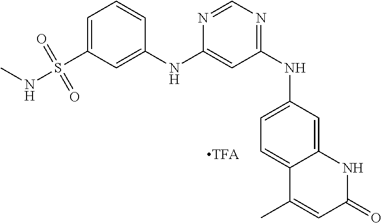 | 7-amino-4-methyl-2(1H)- quinolinone |
| 190 | N-methyl-3-[6-(3,4,5-trifluoro- phenylamino)-pyrimidin-4- ylamino]-benzenesulfonamide hydrochloride | 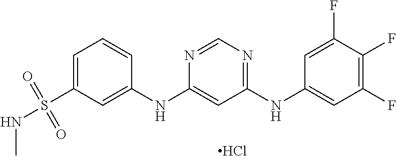 | 3,4,5-trifluoroaniline |
| 191 | 3-[6-(indan-5-ylamino)-pyrimidin-4- ylamino]-N-methyl- N-methyl- benzenesulfonamide trifluoroacetate | 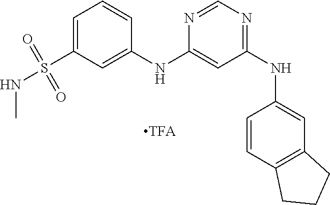 | 2,3-dihydro-1H-inden-5- ylamine |

The following compounds were prepared with procedures analogous to that described in Example 139 using 3-[(6-chloro-4-pyrimidinyl)amino]-N-methyl-4-[(1-methylethyl)sulfonyl]benzenesulfonamide as either the free base or HCl salt and the specified aniline:

| Ex. | Name | Structure | Aniline |
|---|---|---|---|
| 192 | 3-[6-(4-chloro-phenylamino)- pyrimidin-4-ylamino]-N-methyl-4- (propane-2-sulfonyl)- benzenesulfonamide | 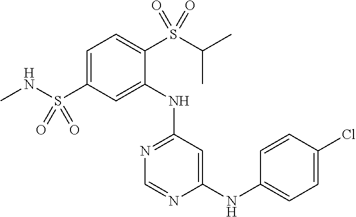 | 4-chloro-aniline |

The following compounds were prepared with procedures analogous to that described in Example 139 using 3-[(6-chloro-4-pyrimidinyl)amino]-N-methyl-4-(methylsulfonyl)benzenesulfonamide as either the free base or HCl salt and the specified aniline:

| Ex. | Name | Structure | Aniline |
|---|---|---|---|
| 193 | 3-(6-(3-bromo-5- methylphenylamino)pyrimidin- 4-ylamino)-N-methyl-4- (methylsulfonyl)benzenesulfon- amide | 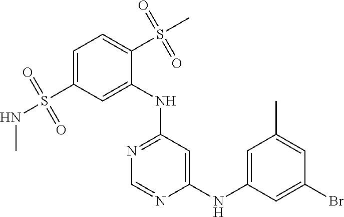 | 3-bromo-5-methylaniline |
| 194 | 3-(6-(1H-indol-6- ylamino)pyrimidin-4- ylamino)-N-methyl-4- (methylsulfonyl)benzenesulfon- amide | 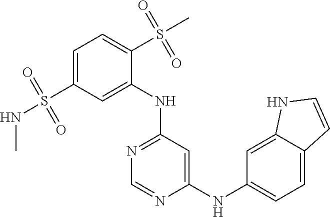 | 1H-indol-6-amine |
| 195 | 3-(6-(3- ethynylphenylamino)pyrimi- din-4-ylamino)-N-methyl- 4- (methylsulfonyl)benzenesul- fonamide | 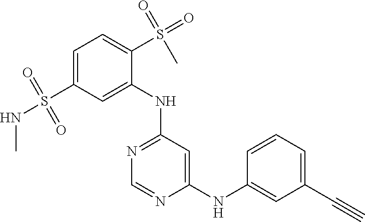 | 3-ethynylaniline |
| 196 | 3-[6-(indan-5-ylamino)- pyrimidin-4-ylamino]-4- methanesulfonyl-N-methyl- benzenesulfonamide | 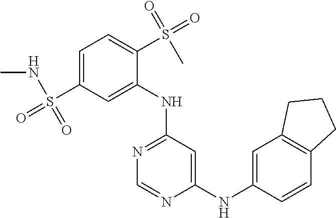 | 2,3-dihydro-1H-inden-5- ylamine |
| 197 | 3-[6-(benzothiazol-6- ylamino)-pyrimidin-4- ylamino]-4-methanesulfonyl- N-methyl- benzenesulfonamide | 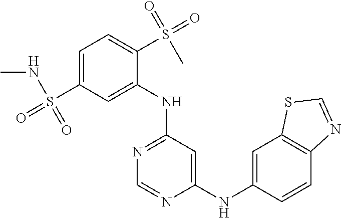 | 1,3-benzothiazol-6- amine |
| 198 | 4-methanesulfonyl-N-methyl- 3-[6-(5-oxo-5,6,7,8- tetrahydro-naphthalen-2- ylamino)-pyrimidin-4- ylamino]- benzensulfonamide | 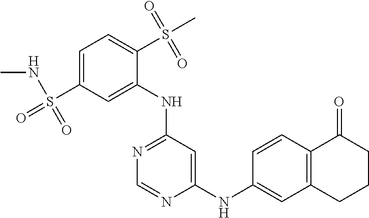 | 6-amino-3,4-dihydro- 1(2H)-naphthalenone |
| 199 | N-methyl-3-(6-(2- methylbenzo[d]thiazol-5- ylamino)pyrimidin-4- ylamino)-4- (methylsulfonyl)benzenesulfon- amide | 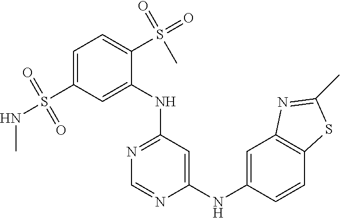 | 2-methyl-1,3- benzothiazol-5-amine |
| 200 | N-methyl-4-(methylsulfonyl)- 3-[(6-{[4-(1H-1,2,4-triazol-1- ylmethyl)phenyl]amino}-4- pyrimidinyl)amino]benzenesul- fonamide | 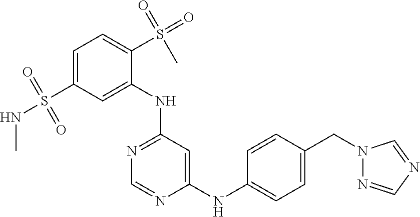 | 4-(1H-1,2,4-triazol-1- ylmethyl)aniline |
| 201 | 3-[6-(1H-indol-5-ylamino)- pyrimidin-4-ylamino]-4- methanesulfonyl-N-methyl- benzenesulfonamide | 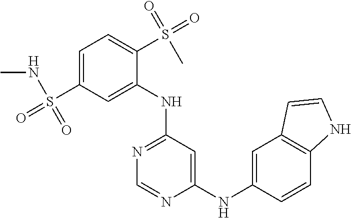 | 1H-indol-5-amine |
| 202 | 4-methanesulfonyl-N-methyl- 3-[6-(2-methyl-4-oxo-4H- chlromen-7-ylamino)- pyrimidin-4-ylamino]- benzenesulfonamide | 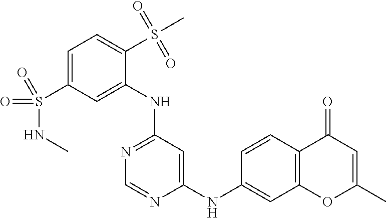 | 7-amino-2-methyl-4H- chromen-4-one |

The following compound was prepared with the procedure analogous to that described in Example 139 using 5-[(6-chloro-4-pyrimidinyl)amino]-2-fluoro-N-methylbenzenesulfonamide as either the free base or HCl salt and the specified aniline:

| Ex. | Name | Structure | Aniline |
|---|---|---|---|
| 203 | 5-({6-[(4- chlorophenyl)amino]-4- pyrimidinyl}amino)-2-fluoro- N- methylbenzenesulfonamide | 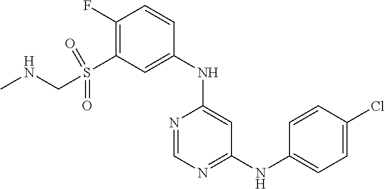 | 4-chloro-aniline |

The following compound was prepared with the procedure analogous to that described in Example 139 using 5-[(6-chloro-4-pyrimidinyl)amino]-2-fluoro-N-methyl-4-[(2,2,2-trifluoro-1-methylethyl)oxy]benzenesulfonamide as either the free base or HCl salt and the specified aniline:

| Ex. | Name | Structure | Aniline |
|---|---|---|---|
| 204 | 5-(6-(4- chlorophenylamino)pyrimidin- 4-ylamino)-2-fluoro-N- methyl-4-(1,1,1- trifluoropropan-2- yloxy)benzenesulfonamide | 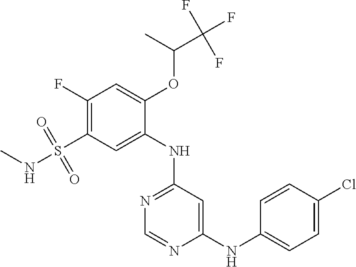 | 4-chloro-aniline |

### Example 205

### 1-{6-[(4-chlorophenyl)amino]-4-pyrimidinyl}-N-methyl-2,3-dihydro-1H-indole-6-sulfonamide hydrochloride

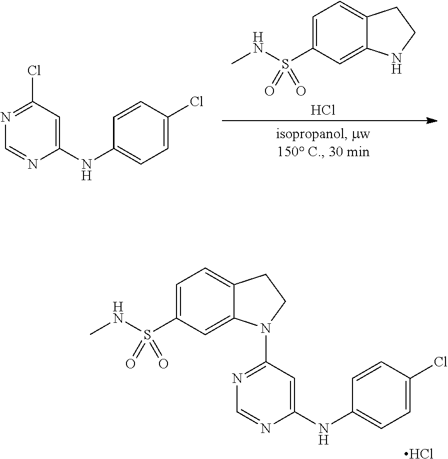

A mixture of 6-chloro-N-(4-chlorophenyl)-4-pyrimidinamine (0.250 g, 1.041 mmol), N-methyl-2,3-dihydro-1H-indole-6-sulfonamide (0.221 g, 1.041 mmol) and a few drops of HCl and isopropanol (2.083 mL) was heated in a microwave reactor at 150° C. for 30 min. The reaction was filtered, washed with Et₂O and the solid collected to afford 1-{6-[(4-chlorophenyl)amino]-4-pyrimidinyl}-N-methyl-2,3-dihydro-1H-indole-6-sulfonamide hydrochloride (0.360 g, 73%) as an off-white solid.

### Example 206

### 3-[(6-{[3,4-bis(methyloxy)phenyl]amino}-4-pyrimidinyl)amino]-N-methylbenzenesulfonamide trifluoroacetate

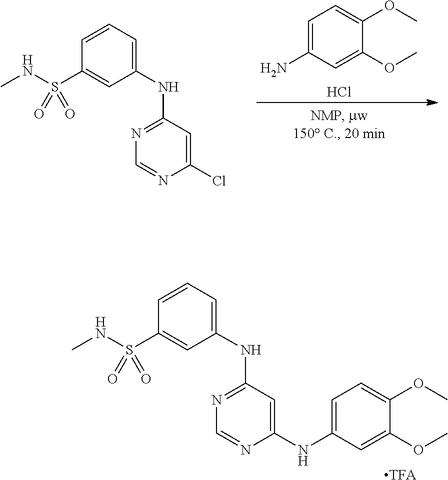

A mixture of 3-[(6-chloro-4-pyrimidinyl)amino]-N-methylbenzenesulfonamide (0.150 g, 0.502 mmol) and 3,4-bis(methyloxy)aniline (0.096 g, 0.648 mmol) in NMP (1.255 mL) was treated with a few drops of concentrated HCl and heated in a microwave reactor at 150° C. for 20 min. Additional aniline (0.038 g, 0.251 mmol) was added and the mixture heated 10 min at 150° C. Reactions were filtered and purified via reverse phase HPLC (Waters, Sunfire 30×100 mm column, 10-90% CH₃CN/Water with 0.1% TFA) to afford 3-[(6-{[3,4-bis(methyloxy)phenyl]amino}-4-pyrimidinyl)amino]-N-methyl benzenesulfonamide trifluoroacetate (0.184 g, 65%) as a brown solid.

The following compounds were prepared with procedures analogous to that described in Example 206 using the specified 3-[(6-chloro-4-pyrimidinyl)amino]-N-methylbenzene-sulfonamide as either the free base, TFA, or HCl salt and the appropriate aniline:

| Ex. | Name | Structure | Aniline |
|---|---|---|---|
| 207 | 3-({6-[(3,4- dichlorophenyl)amino]-4- pyrimidinyl}amino)-N- methylbenzenesulfonamide trifluoroacetate | 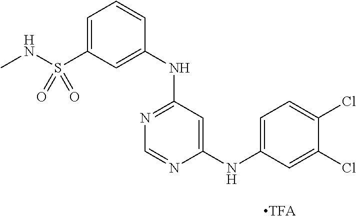 | 3,4-dichloroaniline |
| 208 | 3-({6-[(3,4- dimethylphenyl)amino]-4- pyrimidinyl}amino)-N- methylbenzenesulfonamide trifluoroacetate | 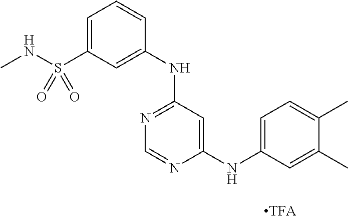 | 3,4-dimethylaniline |
| 209 | N-methyl-3-[(6-{[3-(1- methylethyl)phenyl]amino}-4- pyrimidinyl)amino] benzenesulfonamide | 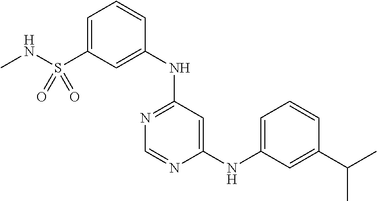 | 3-(1- methylethyl)aniline |
| 210 | 3-[(6-{[3-(1,1- dimethylethyl)phenyl]amino}-4- pyrimidinyl)amino]-N- methylbenzenesulofnamide trifluoroacetate | 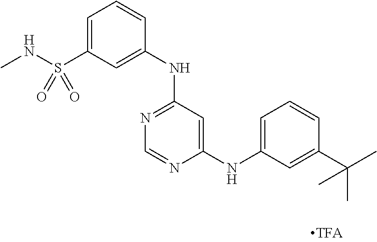 | 3-(1,1- dimethylethyl)aniline |
| 211 | 3-[(6-{[3- (ethyloxy)phenyl]amino}-4- pyrimidinyl)amino]-N- methylbenzenesulfonamide trifluoroacetate | 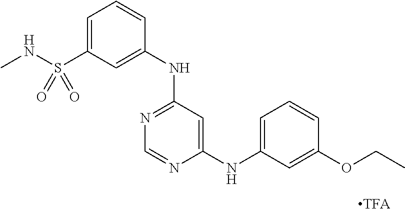 | 3-(ethyloxy)aniline |
| 212 | 3-({6-[(4-fluorophenyl)amino]-4- pyrimidinyl}amino)-N- methylbenzenesulfonamide trifluoroacetate | 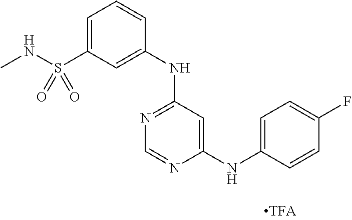 | 4-fluoroaniline |
| 213 | N-methyl-3-[(6-{[3-(1- pyrrolidinyl)phenyl]amino}-4- pyrimidinyl)amino]benzenesulfon amide trifluoroacetate | 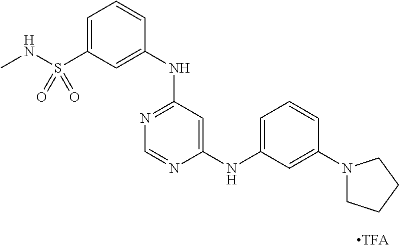 | 3-(1- pyrrolidinyl)aniline |
| 214 | N-methyl-3-[(6-{[3-(4-methyl-1- piperazinyl)phenyl]amino}-4- pyrimidinyl)amino]benzenesulfon amide trifluoroacetate | 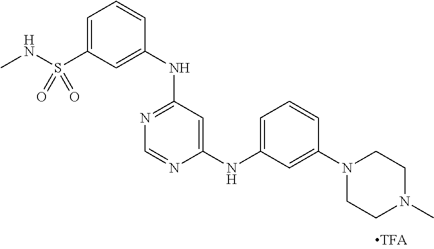 | 3-(4-methyl-1- piperazinyl)aniline |
| 215 | 3-({6-[(3,5- dichlorophenyl)amino]-4- pyrimidinyl}amino)-N- methylbenzenesulfonamide trifluoroacetate | 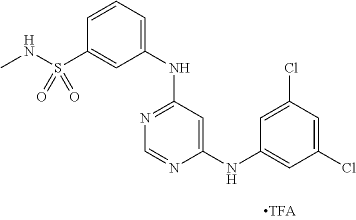 | 3,5-dichloroaniline |
| 216 | N-methyl-3-({6-[(2-oxo-2,3- dihydro-1H-indol-5-yl)amino]-4- pyrimidinyl}amino)benzenesulfon amide trifluoroacetate | 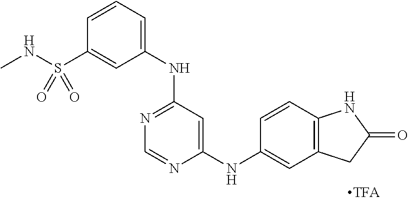 | 5-amino-1,3-dihydro- 2H-indol-2-one |
| 217 | N-methyl-3-({6-[(2-oxo-2,3- dihydro-1,3-benzoxazol-6- yl)amino]-4- pyrimidinyl}amino)benzenesulfon amide trifluoroacetate | 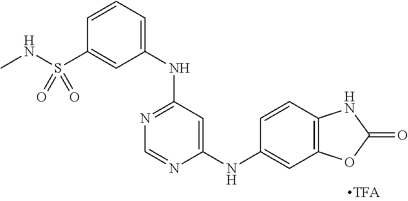 | 6-amino-1,3- benzoxazol-2(3H)-one |
| 218 | N-methyl-3-({6-[(2-oxo-2,3- dihydro-1H-benzimidazol-5- yl)amino]-4- pyrimidinyl}amino)benzenesulfon amide trifluoroacetate | 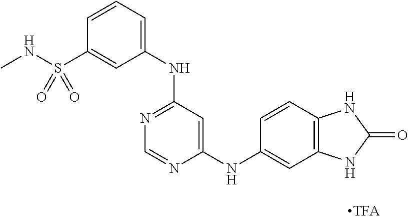 | 5-amino-1,3-dihydro- 2H-benzimidazol-2- one |
| 219 | N-methyl-3-({6-[(2-oxo-1,2,3,4- tetrahydro-7-quinolinyl)amino]-4- pyrimidinyl}amino)benzenesulfon amide trifluoroacetate | 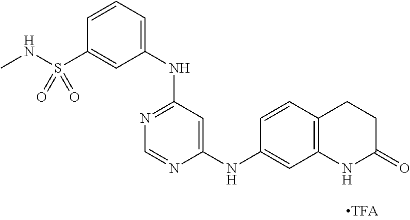 | 7-amino-3,4-dihydro- 2(1H)-quinolinone |
| 220 | 3-({6-[(3-bromo-5- chlorophenyl)amino]-4- pyrimidinyl}amino)-N- methylbenzenesulfonamide trifluoroacetate | 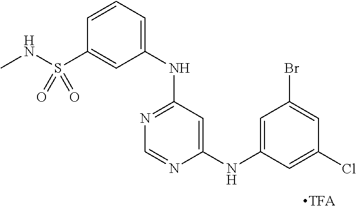 | 3-bromo-5- chloroaniline |
| 221 | 3-({6-[(3,5- dimethylphenyl)amino]-4- pyrimidinyl}amino)-N- methylbenzenesulfonamide trifluoroacetate | 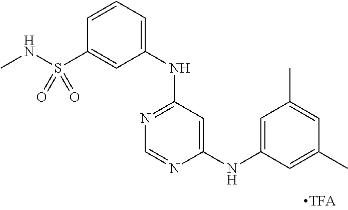 | 3,5-dimethylaniline |
| 222 | N-methyl-3-{[6-({4- [(methylamino)sulfonyl]phenyl} amino)-4- pyrimidinyl]amino}benzenesulfona mide trifluoroacetate | 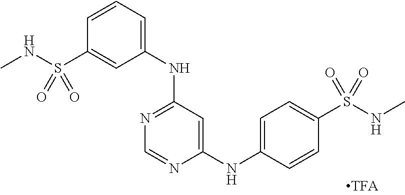 | 4-amino-N- methylbenzenesulfona mide |
| 223 | N-methyl-3-[(6-{[3-(1- pyrrolidinylmethyl)phenyl] amino}-4- pyrimidinyl)amino]benzenesulfon amide trifluoroacetate | 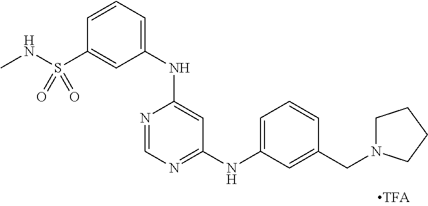 | 3-(1- pyrrolidinylmethyl)aniline |
| 224 | N-methyl-3-({6-[(4-{[2-(4- morpholinyl)ethyl]oxy}phenyl) amino]-4- pyrimidinyl}amino)benzenesulfon amide trifluoroacetate | 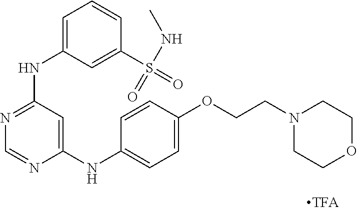 | 4-{[2-(4- morpholinyl)ethyl]oxy} aniline |
| 225 | 3-({6-[(4-{[2- (dimethylamino)ethyl]oxy}phenyl) amino]-4-pyrimidinyl}amino)-N- methylbenzenesulfonamide trifluoroacetate | 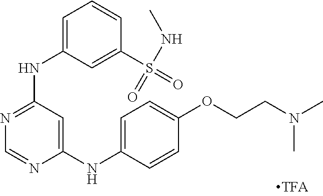 | 4-{[2- (dimethylamino)ethyl]oxy} aniline |
| 226 | N-methyl-3-{[6-({3-[(4-methyl-1- piperazinyl)methyl]phenyl} amino)-4- pyrimidinyl]amino}benzenesulfon amide trifluoroacetate | 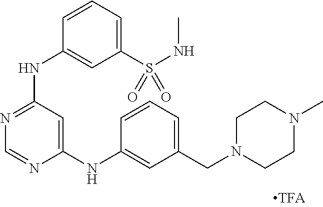 | 3-[(4-methyl-1- piperazinyl)methyl]aniline |
| 227 | N-methyl-3-[(6-{[4- (trifluoromethyl)phenyl]amino)-4- pyrimidinyl)amino]benzenesulfon amide trifluoroacetate | 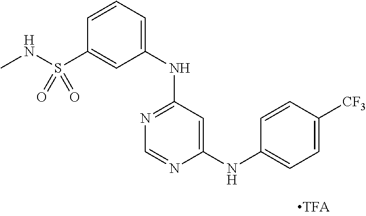 | 4- (trifluoromethyl)aniline |
| 228 | N-methyl-3-[(6-{[4-(1- methylethyl)phenyl]amino}-4- pyrimidinyl)amino]benzenesulfon amide trifluoroacetate | 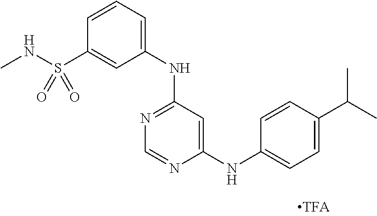 | 4-(1- methylethyl)aniline |
| 229 | N-methyl-3-{[6-({4-[(1- methylethyl)oxy]phenyl}amino)- 4- pyrimidinyl]amino}benzenesulfon amide trifluoroacetate | 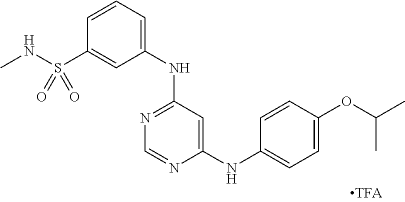 | 4-[(1- methylethyl)oxy]aniline |
| 230 | 3-{[6-({4- [(difluoromethyl)oxy]phenyl} amino)-4-pyrimidinyl]amino}-N- methylbenzenesulfonamide trifluoroacetate | 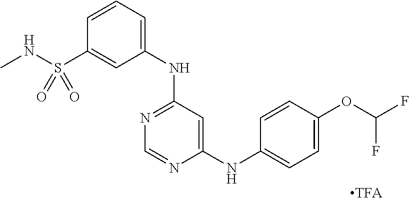 | 4- [(difluoromethyl)oxy]a niline |
| 231 | N-methyl-3-[(6-{[4-(2-oxo-1- pyrrolidinyl)phenyl]amino}-4- pyrimidinyl)amino]benzenesulfon amide trifluoroacetate | 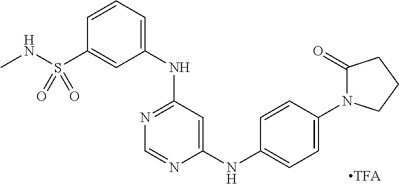 | 1-(4-aminophenyl)-2- pyrrolidinone |
| 232 | 3-[(6-{[3-chloro-4- (methyloxy)phenyl]amino}-4- pyrimidinyl)amino]-N- methylbenzenesulfonamide trifluoroacetate | 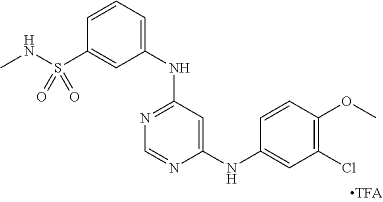 | 3-chloro-4- (methyloxy)aniline |
| 233 | 3-({6-[(4- cyclopropylphenyl)amino]-4- pyrimidinyl}amino)-N- methylbenzenesulfonamide trifluoroacetate | 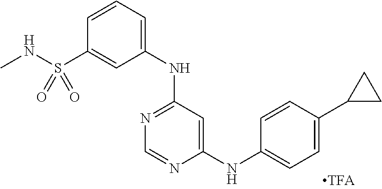 | 4- (cyclopropyloxy)aniline |
| 234 | N-methyl-3-[(6-{[4-(1H-pyrazol-1- yl)phenyl]amino}-4- pyrimidinyl)amino]benzenesulfon amide trifluoroacetate | 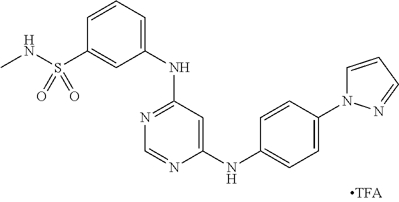 | 4-(1H-pyrazol-1- yl)aniline |
| 235 | 3-[(6-{[4-(3,5-dimethyl-1H- pyrazol-1-yl)phenyl]amino}-4- pyrimidinyl)amino]-N- methylbenzenesulfonamide trifluoroacetate | 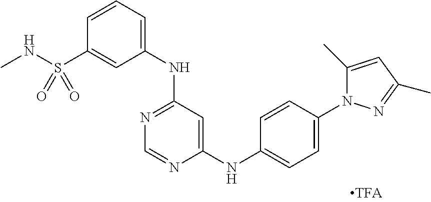 | 4-(3,5-dimethyl-1H- pyraozl-1-yl)aniline |
| 236 | 3-[(6-{[4-chloro-3- (methyloxy)phenyl]amino}-4- pyrimidinyl)amino]-N- methylbenzenesulfonamide trifluoroacetate | 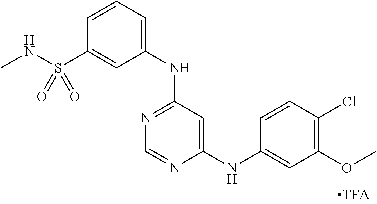 | 4-chloro-3- (methyloxy)aniline |
| 237 | N-methyl-3-[(6-{[4-(2- thienyl)phenyl]amino}-4- pyrimidinyl)amino]benzenesulfon amide trifluoroacetate | 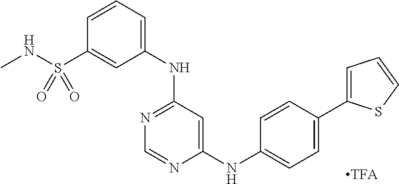 | 4-(2-thienyl)aniline |
| 238 | N-methyl-3-[(6-{[4-(1-methyl-1H- imidazol-1-yl)phenyl]amino}-4- pyrimidinyl)amino]benzenesulfon amide trifluoroacetate | 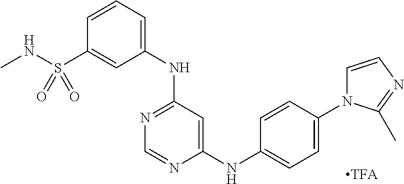 | 4-(2-methyl-1H- imidazol-1-yl)aniline |
| 239 | N-methyl-3-[(6-{[4-(1- methylpropyl)phenyl]amino}-4- pyrimidinyl)amino]benzenesulfon amide trifluoroacetate | 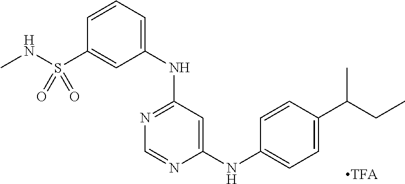 | 4-(1- methylpropyl)aniline |
| 240 | N-methyl-3-{[6-(6- quinolinylamino)-4- pyrimidinyl]amino}benzenesulfon amide | 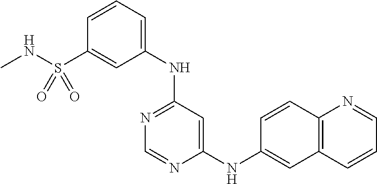 | 6-quinolinamine |
| 241 | N-methyl-3-{[6-({4- [(trifluoromethyl)thio]phenyl}amino)- 4- pyrimidinyl]amino}benzenesulfon amide trifluoroacetate | 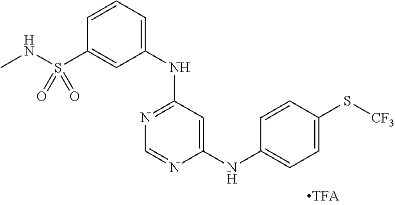 | 4- [(trifluoromethyl)thio]a niline |
| 242 | 3-({6-[(4-bromophenyl)amino]-4- pyrimidinyl}amino)-N- methylbenzenesulfonamide trifluoroacetate | 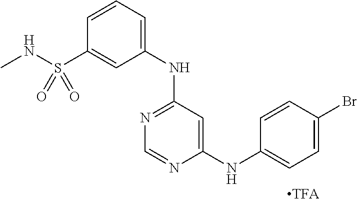 | 4-bromo-aniline |
| 243 | N-methyl-3-[(6-{[4- (methylthio)phenyl]amino}-4- pyrimidinyl)amino]benzenesulfon amide trifluoroacetate | 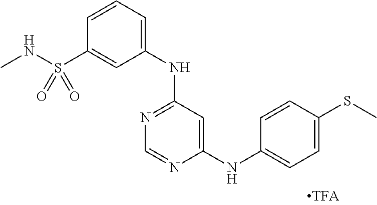 | 4-(methylthio)aniline |
| 244 | N-methyl-3-{[6-({4- [(trifluoromethyl)oxy]phenyl}amino)- 4- pyrimidinyl]amino}benzenesulfon amide trifluoroacetate | 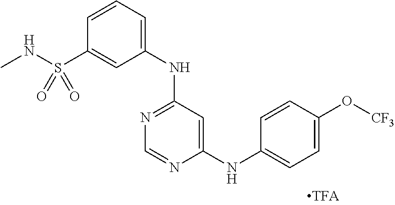 | 4- [(trifluoromethyl)oxy]a niline |

The following compounds were prepared with procedures analogous to that described in Example 206 using the 3-[(6-chloro-4-pyrimidinyl)amino]-4-(dimethylamino)-N-methylbenzene-sulfonamide as either the free base, TFA, or HCl salt and the specified aniline:

| Ex. | Name | Structure | Aniline |
|---|---|---|---|
| 245 | 3-({6-[(4-chlorophenyl)amino]- 4-pyrimidinyl}amino)-4- (dimethylamino)-N- methylbenzenesulfonamide trifluoroacetate | 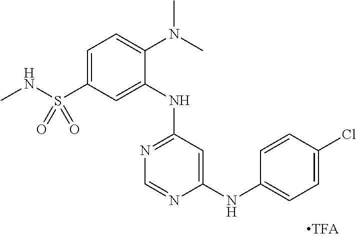 | 4-chloroaniline |
| 246 | 4-(dimethylamino)-N-methyl-3- ({6-[(3-methylphenyl)amino]-4- pyrimidinyl}amino)benzene- sulfonamide trifluoroacetate | 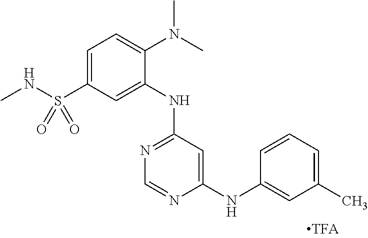 | 3-methylaniline |

The following compound was prepared with procedures analogous to that described in Example 206 using 1-(6-chloro-4-pyrimidinyl)-N-methyl-2,3-dihydro-1H-indole-6-sulfonamide as either the free base, TFA, or HCl salt and the specified aniline:

| Ex. | Name | Structure | Aniline |
|---|---|---|---|
| 247 | N-methyl-1-(6-{[4- (trifluoromethyl)phenyl]amino}- 4-pyrimidinyl)-2,3-dihydro-1H- indole-6-sulfonamide trifluoroacetate | 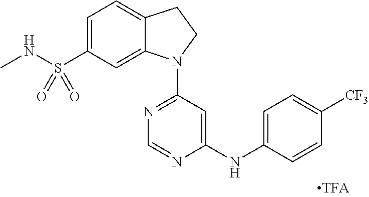 | 4-(trifluoromethyl)aniline |

The following compound was prepared with procedures analogous to that described in Example 206 using 1-(6-chloro-4-pyrimidinyl)-N-methyl-1H-benzimidazole-6-sulfonamide as either the free base, TFA, or HCl salt and the specified aniline:

| Ex. | Name | Structure | Aniline |
|---|---|---|---|
| 248 | 1-{6-[(4-chlorophenyl)amino]- 4-pyrimidinyl}-N-methyl-1H- benzimidazole-6-sulfonamide trifluoroacetate | 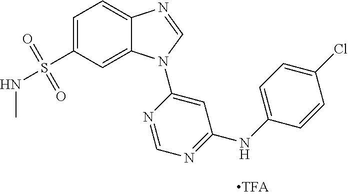 | 4-chloro-aniline |

The following compound was prepared with procedures analogous to that described in Example 206 using N-(5-bromo-6-methyl-2-pyridinyl)-6-chloro-4-pyrimidinamine as either the free base, TFA, or HCl salt and the specified aniline:

| Ex. | Name | Structure | Aniline |
|---|---|---|---|
| 249 | 3-({6-[(5-bromo-6-methyl-2- pyridinyl)amino]-4- pyrimidinyl}amino)-N-methyl-4- [(2,2,2- trifluoroethyl)oxy]benzene- sulfonamide trifluoroacetate | 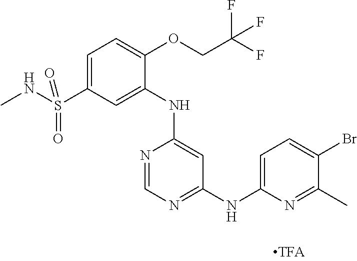 | 3-amino-N-methyl-4- [(2,2,2- trifluoroethyl)oxy]benzene- sulfonamide |

### Example 250

### 3-({6-[(4-chlorophenyl)amino]-4-pyrimidinyl}amino)-N-methyl-4-[(1-methylethyl)oxy]benzenesulfonamide trifluoroacetate

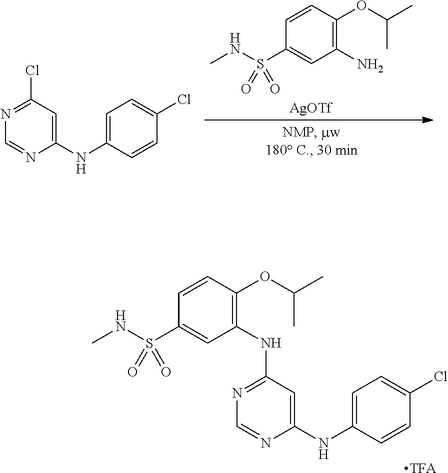

A mixture of 6-chloro-N-(4-chlorophenyl)-4-pyrimidinamine hydrochloride (0.176 g, 0.586 mmol), 3-amino-N-methyl-4-[(1-methylethyl)oxy]benzenesulfonamide (0.179 g, 0.733 mmol) and AgOTf (0.151 g, 0.586 mmol) in NMP (1.562 mL) was heated in a microwave reactor at 180° C. for 30 min. The reaction mixture was filtered and purified by mass directed autoprep (Waters, Sunfire prep C18 OBD, 30×150 mm, 30-70% CH₃CN/water with 0.1% TFA). Concentration of the appropriate fractions yielded 3-({6-[(4-chlorophenyl)amino]-4-pyrimidinyl}amino)-N-methyl-4-[(1-methylethyl)oxy]benzenesulfonamide trifluoroacetate (0.150 g, 43%) as a brown solid.

The following compounds were prepared with procedures analogous to that described in Example 250 using 6-chloro-N-(4-chlorophenyl)-4-pyrimidinamine as the free base, TFA, or HCl salt and the specified aniline:

| Ex. | Name | Structure | Aniline |
|---|---|---|---|
| 251 | 3-({6-[(4-chlorophenyl)amino]-4- pyrimidinyl}amino)-N-methyl-4-(4- morpholinyl)benzenesulfonamide trifluoroacetate | 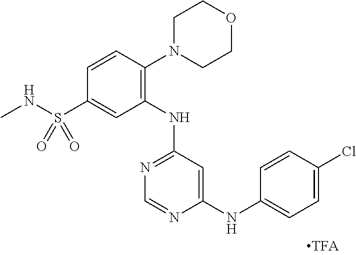 | 3-amino-N-methyl-4-(4- morpholinyl)benzene- sulfonamide |
| 252 | 3-({6-[(4-chlorophenyl)amino]-4- pyrimidinyl}amino)-N-methyl-4- (methyloxy)benzenesulfonamide trifluoroacetate | 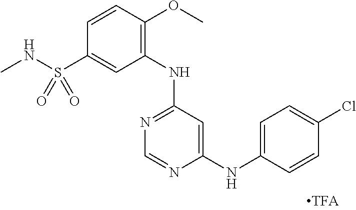 | 3-amino-N-methyl-4- (methyloxy)benzene- sulfonamide |
| 253 | 3-({6-[(4-chlorophenyl)amino]-4- pyrimidinyl}amino)-4- [ethyl(methyl)amino]-N- methylbenzenesulfonamide trifluoroacetate | 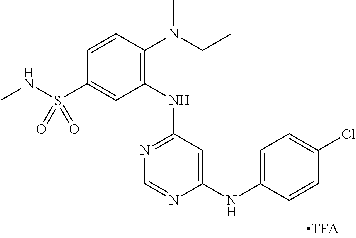 | 3-amino-4- [ethyl(methyl)amino]-N- methylbenzenesulfonamide |
| 254 | 3-({6-[(4-chlorophenyl)amino]-4- pyrimidinyl}amino)-4-hydroxy-N- methylbenzenesulfonamde trifluoroacetate | 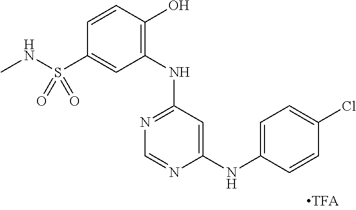 | 3-amino-4-hydroxy-N- methylbenzenesulfonamide |
| 255 | 3-({6-[(4-chlorophenyl)amino]-4- pyrimidinyl}amino)-4-fluoro-N- methylbenzenesulfonamide trifluoroacetate | 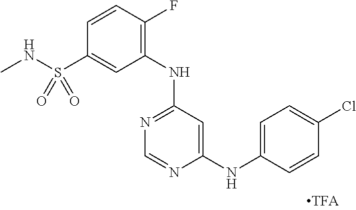 | 3-amino-4-fluoro-N- methylbenzenesulfonamide |
| 256 | 3-({6-[(4-chlorophenyl)amino]-4- pyrimidinyl}amino)-N-methyl-4- (methylthio)benzenesulfonamide trifluoroacetate | 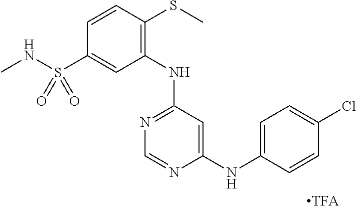 | 3-amino-N-methyl-4- (methylthio)benzene- sulfonamide |
| 257 | 3-({6-[(4-chlorophenyl)amino]-4- pyrimidinyl}amino)-N-methyl-4- [(trifluoromethyl)oxy]benzene- sulfonamide trifluoroacetate | 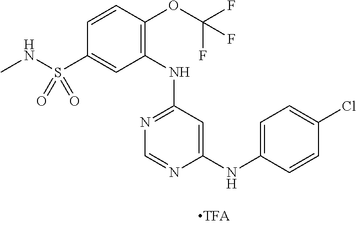 | 3-amino-N-methyl-4- [(trifluoromethyl)oxy] benzenesulfonamide |
| 258 | 3-({6-[(4-chlorophenyl)amino]-4- pyrimidinyl}amino)-N-methyl-4- [(2R)-2-(trifluoromethyl)-1- pyrrolidinyl]benzenesulfonamide trifluoroacetate | 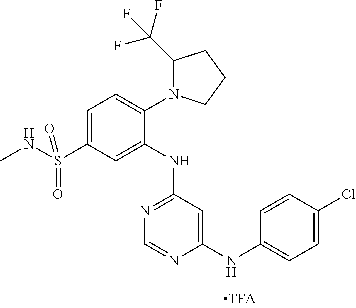 | 3-amino-N-methyl-4- [(2R)-2-(trifluoromethyl)- 1- pyrrolidinyl]benzene- sulfonamide |
| 259 | 3-({6-[(4-chlorophenyl)amino]-4- pyrimidinyl}amino)-4-(3,3-difluoro- 1-pyrrolidinyl)-N- methylbenzenesulfonamide trifluoroacetate | 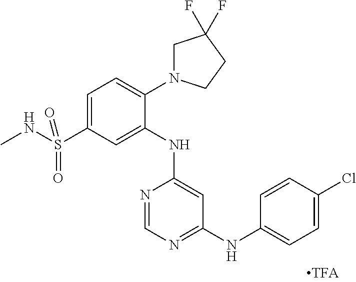 | 3-amino-4-(3,3-difluoro- 1-pyrrolidinyl)-N- methylbenzenesulfonamide |

The following compound was prepared with procedures analogous to that described in Example 250 using 3-[(6-chloro-4-pyrimidinyl)amino]-N-methylbenzene-sulfonamide as the free base, TFA, or HCl salt and the specified aniline:

| Ex. | Name | Structure | Aniline |
|---|---|---|---|
| 260 | N-methyl-3-[(6-{[4-(1,3-oxazol-5- yl)phenyl]amino}-4- pyrimidinyl)amino]benzene sulfonamide trifluoroacetate | 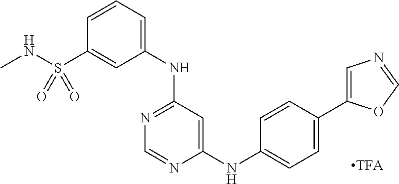 | 4-(1,3-oxazol-5-yl)aniline |

The following compounds were prepared with procedures analogous to that described in Example 250 using 6-chloro-N-(3-methylphenyl)-4-pyrimidinamine as the free base, TFA, or HCl salt and the specified aniline:

| Ex. | Name | Structure | Aniline |
|---|---|---|---|
| 261 | N-methyl-3-({6-[(3- methylphenyl)amino]-4- pyrimidinyl}amino)-4-(4- morpholinyl)benzenesulfonamide trifluoroacetate | 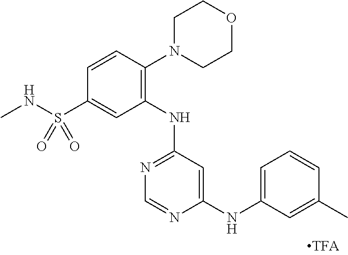 | 3-amino-N-methyl-4-(4- morpholinyl)benzene- sulfonamide |

The following compounds were prepared with procedures analogous to that described in Example 250 using 6-chloro-N-[4-(trifluoromethyl)phenyl]-4-pyrimidinamine as the free base, TFA, or HCl salt and the specified aniline:

| Ex. | Name | Structure | Aniline |
|---|---|---|---|
| 262 | N-methyl-4-(methyloxy)-3-[(6- {[4- (trifluoromethyl)phenyl]amino}- 4-pyrimidinyl)amino]benzene sulfonamide trifluoroacetate | 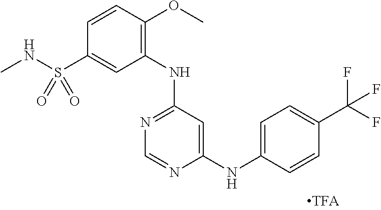 | 3-amino-N-methyl-4- (methyloxy)benzene- sulfonamide |
| 263 | N-methyl-4-(methylthio)-3-[(6- {[4- (trifluoromethyl)phenyl]amino}- 4-pyrimidinyl)amino]benzene sulfonamide trifluoroacetate | 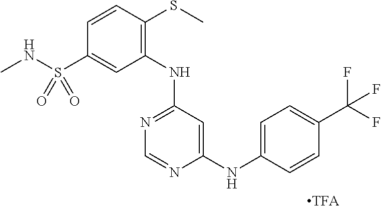 | 3-amino-N-methyl-4- (methylthio)benzene- sulfonamide |

The following compounds were prepared with procedures analogous to that described in Example 250 using N-(3-bromo-5-methylphenyl)-6-chloro-4-pyrimidinamine as the free base, TFA, or HCl salt and the specified aniline:

| Ex. | Name | Structure | Aniline |
|---|---|---|---|
| 264 | 3-({6-[(3-bromo-5- methylphenyl)amino]-4- pyrimidinyl}amino)-N-methyl-4- (methyloxy)benzenesulfonamide trifluoroacetate | 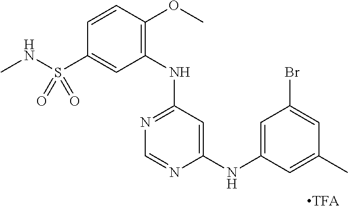 | 3-amino-N-methyl-4- (methyloxy)benzene- sulfonamide |
| 265 | 1-{6-[(3-bromo-5- methylphenyl)amino]-4- pyrimidinyl}-N-methyl-2,3- dihydro-1H-indole-6- sulfonamide trifluoroacetate | 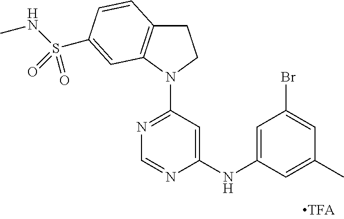 | N-methyl-2,3-dihydro- 1H-indole-6- sulfonamide |

The following compound was prepared with procedures analogous to that described in Example 250 using 6-chloro-N-{4-[(2,2,2-trifluoroethyl)oxy]phenyl}-4-pyrimidinamine as the free base, TFA, or HCl salt and the appropriate aniline:

| Ex | Name | Structure | Aniline |
|---|---|---|---|
| 266 | N-methyl-3-{[6-({4-[(2,2,2- trifluoroethyl)oxy]phenyl}ami no)-4-pyrimidinyl]amino}-4- [(2,2,2- trifluorethyl)thio]benzene- sulfonamide trifluoroacetate | 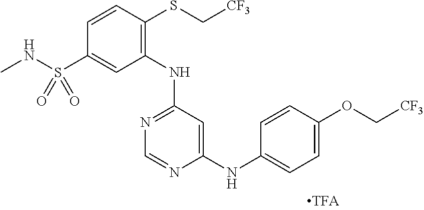 | 3-amino-N-methyl-4- [(2,2,2- trifluoroethyl)thio] benzenesulfonamide |

The following compound was prepared with procedures analogous to that described in Example 250 using 3-[(6-chloro-4-pyrimidinyl)amino]-N-methyl-4-[(trifluoromethyl)oxy]benzenesulfonamide as the free base, TFA, or HCl salt and the specified aniline:

| Ex | Name | Structure | Aniline |
|---|---|---|---|
| 267 | 3-({6-[(3,4- difluorophenyl)amino]-4- pyrimidinyl}amino)-N-methyl- 4- [(trifluoromethyl)oxy]benzene- sulfonamide trifluoroacetate | 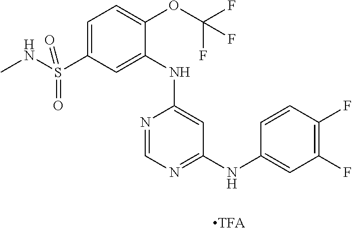 | 3,4-difluoro-aniline |

### Example 268

### N-methyl-3-{[6-(4-pyridinylamino)-4-pyrimidinyl]amino}benzenesulfonamide trifluoroacetate

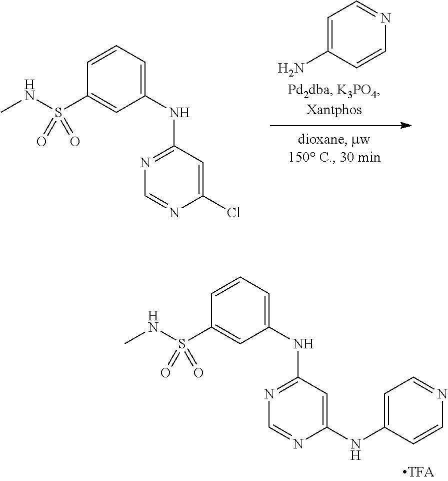

A mixture of 3-[(6-chloro-4-pyrimidinyl)amino]-N-methylbenzenesulfonamide (0.150 g, 0.502 mmol), 4-pyridinamine (0.059 g, 0.628 mmol), Pd₂(dba)₃ (0.009 g, 0.010 mmol), xantphos (11.62 mg, 0.020 mmol) and K₃PO₄ (0.213 g, 1.004 mmol) in 1,4-dioxane (1.255 mL) was heated in a microwave reactor at 150° C. for 30 min. The reaction mixture was loaded onto an ion exchange column (SCX, 5 g, washed with MeOH and eluted with 2 M ammonia in MeOH). Concentration of the ammonia/MeOH fractions yielded 0.243 g of a yellow oil, that was then dissolved in NMP, filtered, and purified by mass directed autoprep (Waters, Sunfire prep C18 OBD, 30×150 mm, 10-50% CH₃CN/water plus 0.1% TFA). Concentration of the appropriate fractions yielded N-methyl-3-{[6-(4-pyridinylamino)-4-pyrimidinyl]amino}benzenesulfonamide trifluoroacetate (0.053 g, 21%) as a white solid.

The following compounds were prepared with procedures analogous to that described in Example 268 using 3-[(6-chloro-4-pyrimidinyl)amino]-N-methylbenzene-sulfonamide in its free base, TFA, or HCl salt form and the specified amine:

| Ex. | Name | Structure | Amine |
|---|---|---|---|
| 269 | N-methyl-3-{[6-(3- pyridinylamino)-4- pyrimidinyl]amino} benzenesulfonamide | 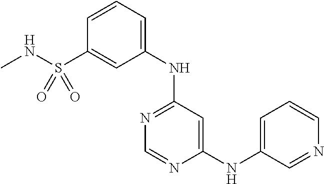 | 3-pyridinamine |
| 270 | N-methyl-3-({6-[(5-methyl-3- pyridinyl)amino]-4- pyrimidinyl}amino) benzenesulfonamide | 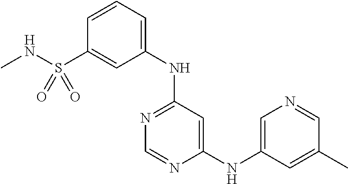 | 5-methyl-3-pyridinamine |
| 271 | N-methyl-3-{[6-(2- pyridinylamino)-4- pyrimidinyl]amino} benzenesulfonamide | 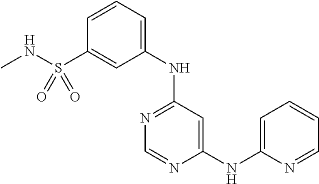 | 2-pyridiniamine |
| 272 | N-methyl-5-{[6-({3- [(methylamino)sulfonyl]phenyl} amino)-4-pyrimidinyl]amino}-3- pyridinesulfonamide | 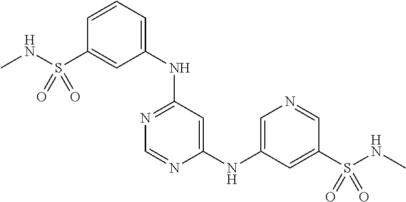 | 5-amino-N-methyl-3- pyridinesulfonamide |
| 273 | 3-({6-[(5-chloro-2- pyridinyl)amino]-4- pyrimidinyl}amino)-N- methylbenzenesulfonamide trifluoroacetate | 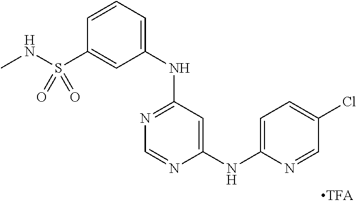 | 5-chloro-2-pyridinamine |
| 274 | N-methyl-3-{[6-(1,3-thiazol-2- ylamino)-4- pyrimidinyl]amino} benzenesulfonamide trifluoroacetate | 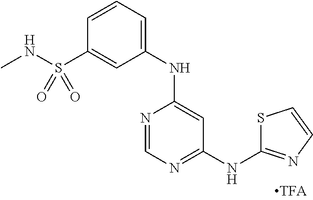 | 1,3-thiazol-2-amine |
| 275 | N-methyl-3-[(6-{[5- (trifluoromethyl)-2- pyridinyl]amino}-4- pyrimidinyl)amino] benzenesulfonamide trifluoroacetate | 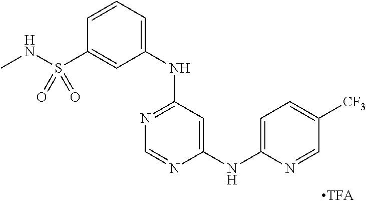 | 5-(trifluoromethyl)-2- pyridinamine |
| 276 | N-methyl-3-({6-[(5-methyl-1,3- thiazol-2-yl)amino]-4- pyrimidinyl}amino) benzenesulfonamide | 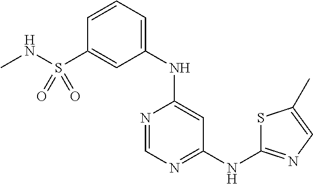 | 5-methyl-1,3-thiazol-2- amine |
| 277 | N-methyl-3-{[6-(1,3,4- thiadiazol-2-ylamino)-4- pyrimidinyl]amino} benzenesulfonamide | 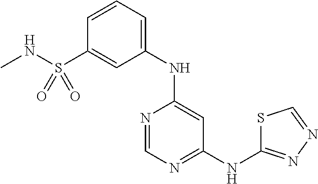 | 1,3,4-thiadiazol-2-amine |
| 278 | 3-{[6-(3-isoquinolinylamino)-4- pyrimidinyl]amino}-N- methylbenzenesulfonamide | 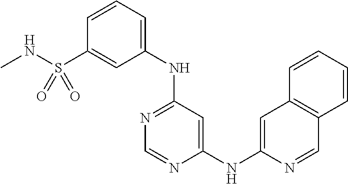 | 3-isoquinolinamine |
| 279 | N-methyl-3-{[6-(2- quinolinylamino)-4- pyrimidinyl]amino} benzenesulfonamide | 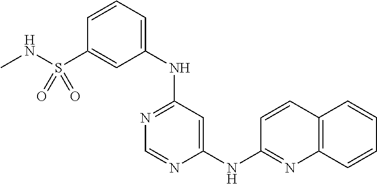 | 2-quinolinamine |
| 280 | N-methyl-3-{[6-(1,3-oxazol-2- ylamino)-4- pyrimidinyl]amino} benzenesulfonamide trifluoroacetate | 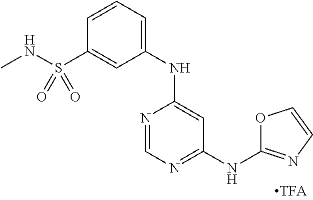 | 1,3-oxazol-2-amine |
| 281 | N-methyl-3-[(6-{[4- (trifluoromethyl)-1,3-thiazol-2- yl]amino}-4- pyrimidinyl)amino] benzenesulfonamide | 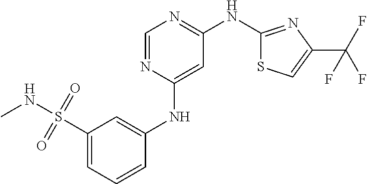 | 4-(trifluoromethyl)-1,3- thiazol-2-amine |
| 282 | methyl (2-{[6-({3- [(methylamino)sulfonyl]phenyl} amino)-4-pyrimidinyl]amino}- 1,3-thiazol-4-yl)acetate trifluoroacetate | 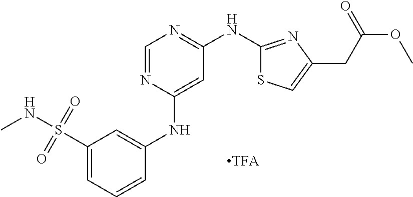 | methyl (2-amino-1,3- thiazol-4-yl)acetate |
| 283 | N-methyl-3-[(6-{[4-(1- methylethyl)-1,3-thiazol-2- yl]amino}-4- pyrimidinyl)amino] benzenesulfonamide trifluoroacetate | 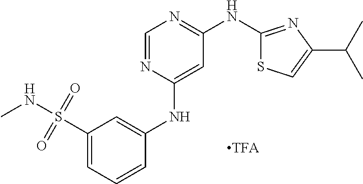 | 4-(1-methylethyl)-1,3- thiazol-2-amine |
| 284 | N-methyl-3-({6-[(4-methyl-1,3- oxazol-2-yl)amino]-4- pyrimidinyl}amino) benzenesulfonamide | 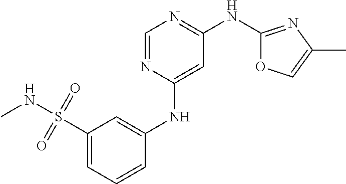 | 4-methyl-1,3-oxazol-2- amine |

The following compounds were prepared with procedures analogous to that described in Example 268 using 3-[(6-chloro-4-pyrimidinyl)amino]-N-methyl-4-(methyloxy)benzenesulfonamide in its free base, TFA, or HCl salt form and the specified amine using either K₃PO₄ or K₂CO₃ as the base:

| Ex. | Name | Structure | Amine |
|---|---|---|---|
| 285 | N-methyl-4-(methyloxy)-3-{[6- (2-pyridinylamino)-4- pyrimidinyl]amino} benzenesulfonamide trifluoroacetate | 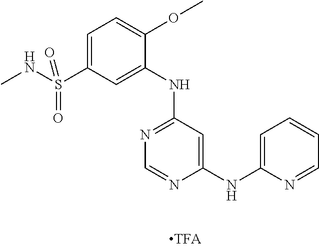 | 2-pyridinamine |
| 286 | 3-({6-[(5-chloro-2- pyridinyl)amino]-4- pyrimidinyl}amino)-N- methyl-4-(methyloxy) benzenesulfonamide trifluoroacetate | 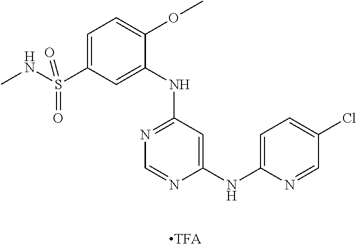 | 5-chloro-2-pyridinamine |

The following compounds were prepared with procedures analogous to that described in Example 268 using 3-[(6-chloro-4-pyrimidinyl)amino]-N-methyl-4-[(2,2,2-trifluoroethyl)oxy]benzenesulfonamide as either the free base or HCl salt and the specified amine:

| Ex. | Name | Structure | Amine |
|---|---|---|---|
| 287 | 3-({6-[(5-chloro-2- pyridinyl)amino]-4- pyrimidinyl}amino)-N- methyl-4-[(2,2,2- trifluoroethyl)oxy] benzenesulfonamide trifluoroacetate | 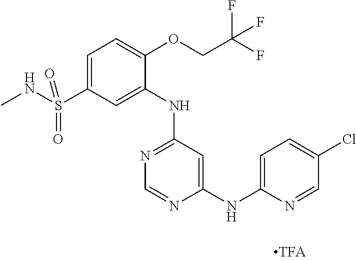 | 5-chloro-2-pyridinamine |
| 288 | N-methyl-3-{[6-(2- pyridinylamino)-4- pyrimidinyl]amino}-4-[(2,2,2- trifluoroethyl)oxy] benzenesulfonamide trifluoroacetate | 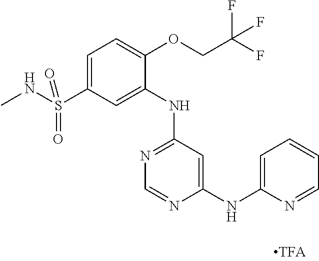 | 2-pyridinamine |

The following compounds were prepared with procedures analogous to that described in Example 268 using 3-[(6-chloro-4-pyrimidinyl)amino]-N-methyl-4-(methylthio)benzenesulfonamide as either the free base or HCl salt and the specified amine:

| Ex. | Name | Structure | Amine |
|---|---|---|---|
| 289 | 3-({6-[(5-chloro-2- pyridinyl)amino]-4- pyrimidinyl}amino)- N-methyl-4-(methylthio) benzenesulfonamide trifluoroacetate | 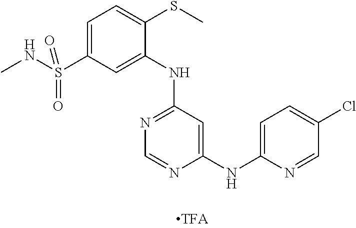 | 5-chloro-2-pyridinamine |

The following compounds were prepared with procedures analogous to that described in Example 268 using 1-(6-chloro-4-pyrimidinyl)-N-methyl-2,3-dihydro-1H-indole-6-sulfonamide as either the free base or HCl salt and the specified amine using K₂CO₃ as the base:

| Ex. | Name | Structure | Amine |
|---|---|---|---|
| 290 | 1-{6-[(5-chloro-2- pyridinyl)amino]-4- pyrimidinyl}-N-methyl-2,3- dihydro-1H-indole-6- sulfonamide trifluoroacetate | 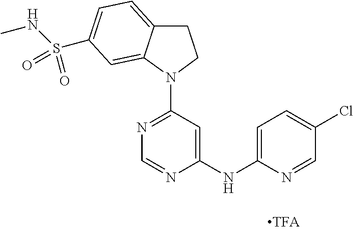 | 5-chloro-2-pyridinamine |

### Example 291

### N-methyl-4-[(2,2,2-trifluoroethyl)oxy]-3-[(6-{[5-(trifluoromethyl)-2-pyridinyl]amino}-4-pyrimidinyl)amino]benzenesulfonamide trifluoroacetate

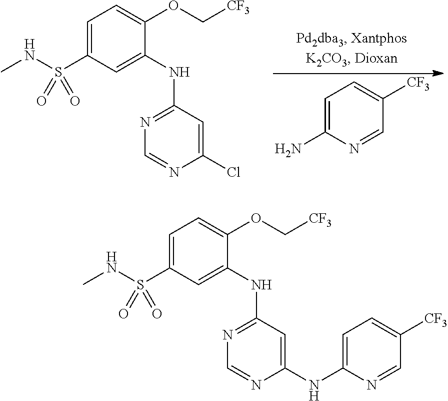

A mixture of 3-[(6-chloro-4-pyrimidinyl)amino]-N-methyl-4-[(2,2,2-trifluoroethyl)oxy]benzenesulfonamide (330 mg, 0.832 mmol), 5-(trifluoromethyl)-2-pyridinamine (539 mg, 3.33 mmol), Pd₂ dba₃ (15.23 mg, 0.017 mmol), Xantphos (19.25 mg, 0.033 mmol) and potassium carbonate (1149 mg, 8.32 mmol) in 1,4-dioxane (3327 μl) was heated in the microwave at 180° C. for a total of 90 min. The reaction was filtered and the filtrate loaded onto a SCX (10 g, washed with MeOH and eluted with 2M ammonia in MeOH). Concentration of the ammonia/MeOH fractions yielded a brown solid which was subsequently dissolved in DMSO/MeOH and purified by mass directed autoprep (Waters, Sunfire prep C18 OBD, 30×150 mm, 20-60% CH₃CN/water plus 0.1% TFA) to give N-methyl-4-[(2,2,2-trifluoroethyl)oxy]-3-[(6-{[5-(trifluoromethyl)-2-pyridinyl]amino}-4-pyrimidinyl)amino]benzenesulfonamide trifluoroacetate (33 mg, 5.9%) as a pale yellow solid.

The following compounds were prepared with procedures analogous to that described in Example 291 using 3-[(6-chloro-4-pyrimidinyl)amino]-N-methyl-4-[(2,2,2-trifluoroethyl)oxy]benzenesulfonamide as either the free base or HCl salt and the specified amine:

| Ex. | Name | Structure | Amine |
|---|---|---|---|
| 292 | N-methyl-3-{[6-(4- pyridinylamino)-4- pyrimidinyl]amino}-4-[(2,2,2- trifluoroethyl)oxy] benzenesulfonamide trifluoroacetate | 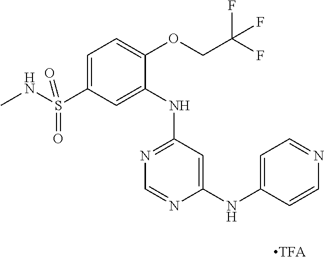 | 4-pyridinamine |
| 293 | 3-({6-[(3-fluoro-2- pyridinyl)amino]-4- pyrimidinyl}amino)-N-methyl- 4-[(2,2,2- trifluoroethyl)oxy] benzenesulfonamide trifluoroacetate | 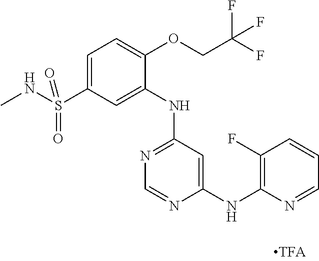 | 3-fluoro-2-pyridinamine |
| 294 | 3-({6-[(5-cyano-2- pyridinyl)amino]-4- pyrimidinyl}amino)-N-methyl- 4-[(2,2,2- trifluoroethyl)oxy] benzenesulfonamide trifluoroacetate | 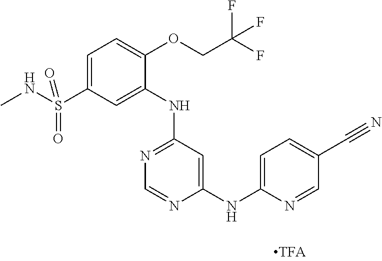 | 6-amino-3- pyridinecarbonitrile |
| 295 | N-methyl-3-{[6-(4- pyrimidinylamino)-4- pyrimidinyl]amino}-4-[(2,2,2- trifluoroethyl)oxy] benzenesulfonamide | 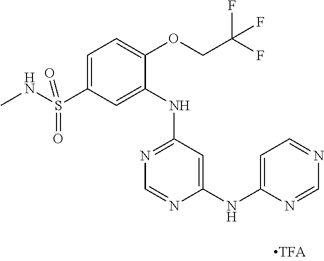 | 4-pyrimidinamine |
| 296 | 3-({6-[(5-chloro-3-fluoro-2- pyridinyl)amino]-4- pyrimidinyl}amino)-N-methyl- 4-[(2,2,2- trifluoroethyl)oxy] benzenesulfonamide | 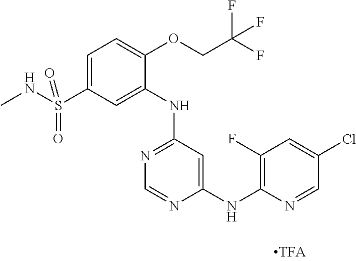 | 5-chloro-3-fluoro-2- pyridinamine |
| 297 | N-methyl-4-[(2,2,2- trrfluoroethyl)oxy]-3-[(6-{[6- (trifluoromethyl)-3- pyridinyl]amino}-4- pyrimidinyl)amino] benzenesulfonamide | 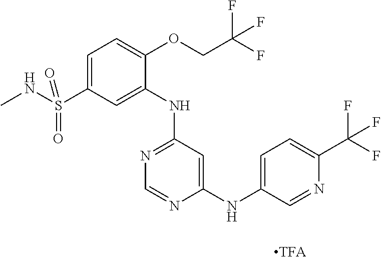 | 6-(trifluoromethyl)-3- pyridinamine |
| 298 | 3-({6-[(5-chloro-4-methyl-2- pyridinyl)amino]-4- pyrimidinyl}amino)-N-methyl- 4-[(2,2,2- trifluoroethyl)oxy] benzenesulfonamide trifluoroacetate | 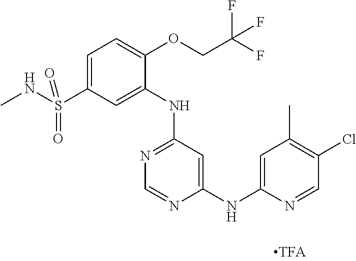 | 5-chloro-4-methyl-2- pyridinamine |
| 299 | 3-({6-[(4,5-dichloro-2- pyridinyl)amino]-4- pyrimidinyl}amino)-N-methyl- 4-[(2,2,2- trifluoroethyl)oxy] benzenesulfonamide trifluoroacetate | 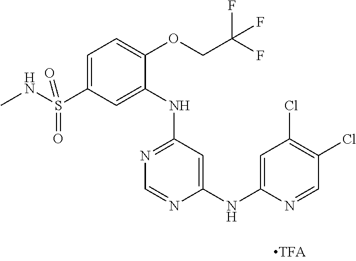 | 4,5-dichloro-2- pyridinamine |
| 300 | 3-({6-[(5-chloro-6-methyl-2- pyridinyl)amino]-4- pyrimidinyl}amino)-N-methyl- 4-[(2,2,2- trifluoroethyl)oxy] benzenesulfonamide trifluoroacetate | 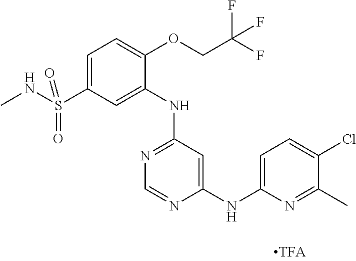 | 5-chloro-6-methyl-2- pyridinamine |
| 301 | 3-(6-(5-isopropylpyridin-2- ylamino)pyrimidin-4-ylamino)- N-methyl-4-(2,2,2- trifluoroethoxy) benzenesulfonamide trifluoroacetate | 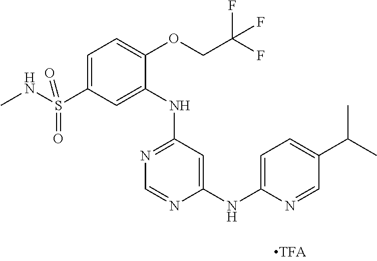 | 5-(1-methylethyl)-2- pyridinamine |

The following compounds were prepared with procedures analogous to that described in Example 291 using 3-[(6-chloro-4-pyrimidinyl)amino]-4-fluoro-N-methylbenzenesulfonamide in its free base, TFA, or HCl salt form and the specified amine:

| Ex. | Name | Structure | Amine |
|---|---|---|---|
| 302 | 3-({6-[(5-chloro-2- pyridinyl)amino]-4- pyrimidinyl}amino)-4-fluoro- N-methylbenzenesulfonamide trifluoroacetate | 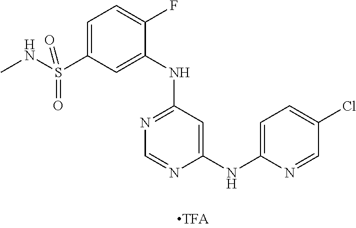 | 5-chloro-2-pyridinamine |
| 303 | 4-fluoro-N-methyl-3-[(6-{[5- (trifluoromethyl)-2- pyridinyl]amino}-4-pyrimidinyl) amino]benzenesulfonamide trifluoroacetate | 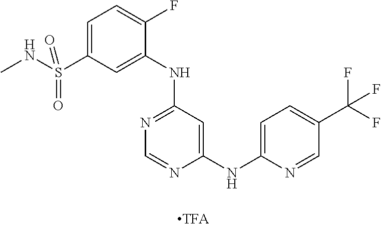 | 5-(trifluoromethyl)-2- pyridinamine |

The following compound was prepared with procedures analogous to that described in Example 291 using 4-chloro-3-[(6-chloro-4-pyrimidinyl)amino]-N-methylbenzenesulfonamide in its free base, TFA, or HCl salt form and the specified amine:

| Ex. | Name | Structure | Amine |
|---|---|---|---|
| 304 | 4-chloro-3-({6-[(5-chloro-2- pyridinyl)amino]-4- pyrimidinyl}amino)-N- methylbenzenesulfonamide trifluoroacetate | 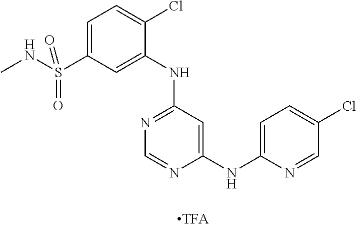 | 5-chloro-2-pyridinamine |

The following compounds were prepared with procedures analogous to that described in Example 291 using 3-[(6-chloro-4-pyrimidinyl)amino]-N-methyl-4-(methylsulfonyl)benzenesulfonamide in its free base, TFA, or HCl salt form and the specified amine:

| Ex. | Name | Structure | Amine |
|---|---|---|---|
| 305 | 3-({6-[(5-chloro-2- pyridinyl)amino]-4- pyrimidinyl}amino)- N-methyl-4- (methylsulfonyl) benzenesulfonamide | 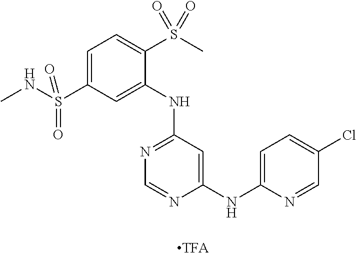 | 5-chloro-2-pyridinamine |
| 306 | N-methyl-4-(methylsulfonyl)- 3-[(6-{[5-(trifluoromethyl)-2- pyridinyl]amino}-4- pyrimidinyl)amino] benzenesulfonamide | 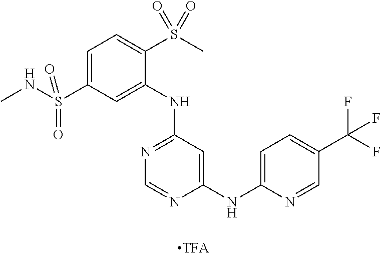 | 5-(trifluoromethyl)-2- pyridinamine |
| 307 | N-methyl-4-(methylsulfonyl)- 3-{[6-(6-quinolinylamino)-4- pyrimidinyl]amino} benzenesulfonamide | 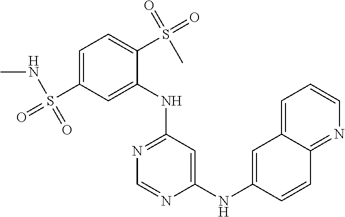 | 6-quinolinamine |

The following compounds were prepared with procedures analogous to that described in Example 291 using 3-[(6-chloro-4-pyrimidinyl)amino]-N-methyl-4-[(2,2,2-trifluoro-1-methylethyl)oxy]benzenesulfonamide as either the free base or HCl salt and the specified amine:

| Ex. | Name | Structure | Amine |
|---|---|---|---|
| 308 | 3-({6-[(5-chloro-2- pyridinyl)amino]-4- pyrimidinyl}amino)-N- methyl-4-[(2,2,2-trifluoro-1- methylethyl)oxy] benzenesulfonamide trifluoroacetate | 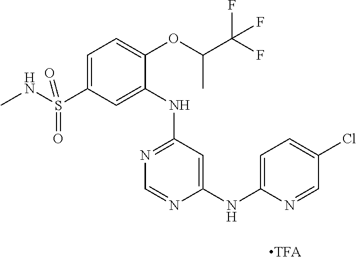 | 5-chloro-2-pyridinamine |
| 309 | N-methyl-4-[(2,2,2-trifluoro- 1-methylethyl)oxy]-3-[(6-{[5- (trifluoromethyl)-2- pyridinyl]amino}-4- pyrimidinyl)amino] benzenesulfonamide | 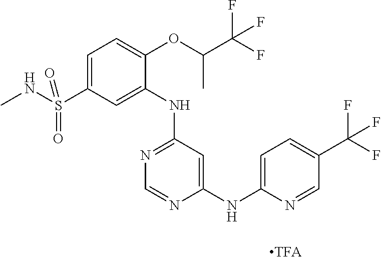 | 5-(trifluoromethyl)-2- pyridinamine |

The following compounds were prepared with procedures analogous to that described in Example 291 using 3-[(6-chloro-4-pyrimidinyl)amino]-4-[(1,1-dimethylethyl)sulfonyl]-N-methylbenzenesulfonamide as either the free base or HCl salt and the specified amine:

| Ex. | Name | Structure | Amine |
|---|---|---|---|
| 310 | 4-(tert-butylsulfonyl)-N- methyl-3-(6-(5- (trifluoromethyl)pyridin-2- ylamino)pyrimidin-4- ylamino)benzenesulfonamide trifluoroacetate | 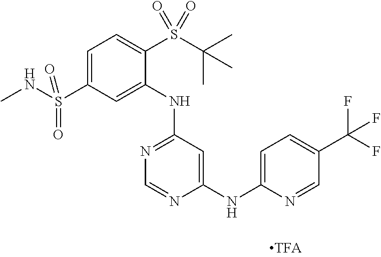 | 5-(trifluoromethyl)-2- pyridinamine |
| 311 | 4-(tert-butylsulfonyl)-3-(6-(5- chloropyridin-2- ylamino)pyrimidin-4- ylamino)-N- methylbenzenesulfonamide trifluoroacetate | 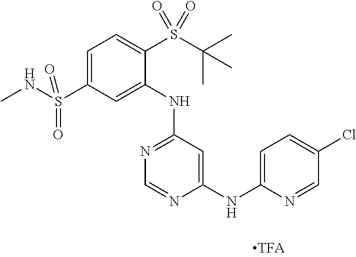 | 5-chloro-2-pyridinamine |

The following compounds were prepared with procedures analogous to that described in Example 291 using 3-[(6-chloro-4-pyrimidinyl)amino]-N-methyl-4-[(1-methylethyl)sulfonyl]benzenesulfonamide as either the free base or HCl salt and the specified amine:

| Ex. | Name | Structure | Amine |
|---|---|---|---|
| 312 | N-methyl-4-(propane-2- sulfonyl)-3-[6-(5- trifluoromethyl-pyridin-2- ylamino)-pyrimidin-4- ylamino]- benzenesulfonamide | 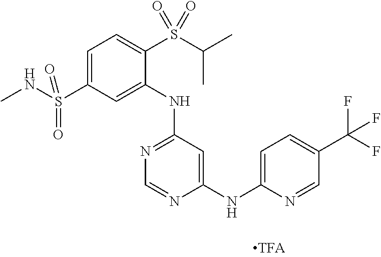 | 5-(trifluoromethyl)-2- pyridinamine |
| 313 | 3-[6-(5-chloro-pyridin-2- ylamino)-pyrimidin-4- ylamino]-N-methyl-4- (propane-2-sulfonyl)- benzenesulfonamide | 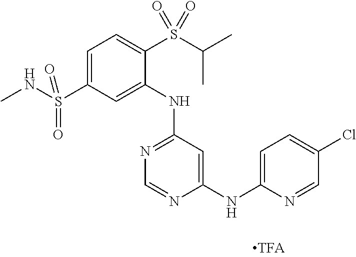 | 5-chloro-2-pyridinamine |

The following compounds were prepared with procedures analogous to that described in Example 291 using 3-[(6-chloro-4-pyrimidinyl)amino]-N-methyl-4-[(trifluoromethyl)oxy]benzenesulfonamide as either the free base or HCl salt and the specified amine:

| Ex. | Name | Structure | Amine |
|---|---|---|---|
| 314 | 3-({6-[(5-chloro-2- pyridinyl)amino]-4- pyrimidinyl}amino)-N- methyl-4- [(trifluoromethyl)oxy] benzenesulfonamide trifluoroacetate | 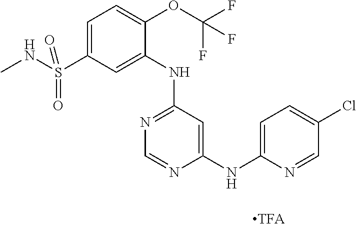 | 5-chloro-2-pyridinamine |

The following compounds were prepared with procedures analogous to that described in Example 291 using 1-(6-chloro-4-pyrimidinyl)-N,3,3-trimethyl-2,3-dihydro-1H-indole-6-sulfonamide as either the free base or HCl salt and the specified amine:

| Ex. | Name | Structure | Amine |
|---|---|---|---|
| 315 | 1-[6-(5-chloro-pyridin-2- ylamino)-pyrimidin-4-yl]-3,3- dimethyl-2,3-dihydro-1H- indole-6-sulfonic acid methylamide trifluoroacetate | 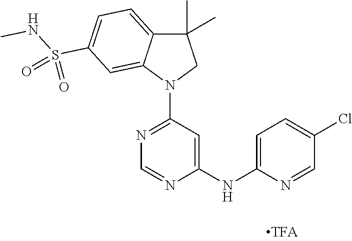 | 5-chloro-2-pyridinamine |

The following compounds were prepared with procedures analogous to that described in Example 291 using 5-[(6-chloro-4-pyrimidinyl)amino]-2-fluoro-N-methyl-4-[(2,2,2-trifluoro-1-methylethyl)oxy]benzenesulfonamide as either the free base or HCl salt and the specified amine:

| Ex. | Name | Structure | Amine |
|---|---|---|---|
| 316 | 5-(6-(5-chloropyridin-2- ylamino)pyrimidin-4-ylamino)- 2-fluoro-N-methyl-4-(1,1,1- trifluoropropan-2- yloxy)benzenesulfonamide trifluoroacetate | 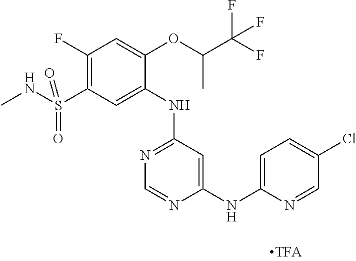 | 5-chloro-2-pyridinamine |

The following compounds were prepared with procedures analogous to that described in Example 291 using 5-[(6-chloro-4-pyrimidinyl)amino]-2-fluoro-N-methyl-4-(methylsulfonyl)benzenesulfonamide as either the free base or HCl salt and the specified amine:

| Ex. | Name | Structure | Amine |
|---|---|---|---|
| 317 | 5-[6-(5-chloro-pyridin-2- ylamino)-pyrimidin-4- ylamino]-2-fluoro-4- methanesulfonyl-N-methyl- benzenesulfonamide trifluoroacetate | 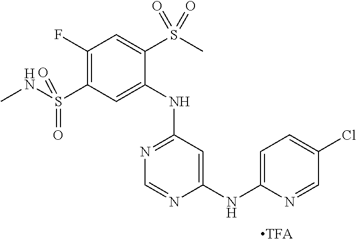 | 5-chloro-2-pyridinamine |

### Example 318

### 5-({6-[(5-chloro-2-pyridinyl)amino]-4-pyrimidinyl}amino)-2-fluoro-N-methyl-4-[(2,2,2-trifluoroethyl)oxy]benzenesulfonamide trifluoroacetate

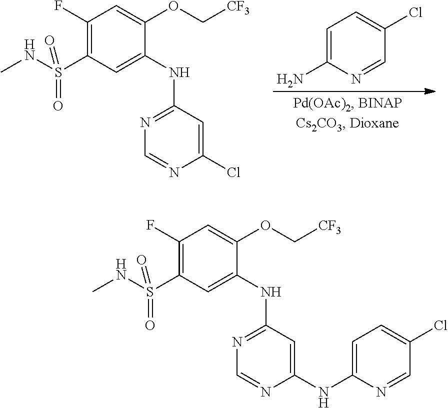

A mixture of 5-[(6-chloro-4-pyrimidinyl)amino]-2-fluoro-N-methyl-4-[(2,2,2-trifluoroethyl)oxy]benzenesulfonamide (550 mg, 1.326 mmol), 5-chloro-2-pyridinamine (682 mg, 5.30 mmol), Cs₂CO₃ (1296 mg, 3.98 mmol), Pd(OAc)₂ (5.95 mg, 0.027 mmol) and BINAP (16.51 mg, 0.027 mmol) in 1,4-dioxane (3315 μl) was heated in the microwave at 150° C. for 30 min. The reaction mixture was concentrated, dissolved in NMP, filtered and purified by MDAP (Waters, Sunfire 30×150 mm, 20-60% acetonitrile+0.1% TFA:water+0.1% TFA) to give 158 mg of a white solid, 90% pure by NMR. This solid was then purified by silica SPE (5 g, eluted with 50-50 CH₂Cl₂:Et₂O, 25-75 CH₂Cl₂:Et₂O, Et₂O, EtOAc then MeOH). Concentration of the appropriate fractions yielded 5-({6-[(5-chloro-2-pyridinyl)amino]-4-pyrimidinyl}amino)-2-fluoro-N-methyl-4-[(2,2,2-trifluoroethyl)oxy]benzenesulfonamide trifluoroacetate (51 mg, 5.8%) as a white solid.

The following compound was prepared with procedures analogous to that described in Example 318 using 5-[(6-chloro-4-pyrimidinyl)amino]-2-fluoro-N-methyl-4-[(2,2,2-trifluoroethyl)oxy]benzenesulfonamide as either the free base or HCl salt, and the specified amine:

| Ex. | Name | Structure | Amine |
|---|---|---|---|
| 319 | 2-fluoro-N-methyl-4-[(2,2,2- trifluoroethyl)oxy]-5-[(6-{[5- (trifluoromethyl)-2- pyridinyl]amino}-4- pyrimidinyl)amino]benzene- sulfonamide trifluoroacetate | 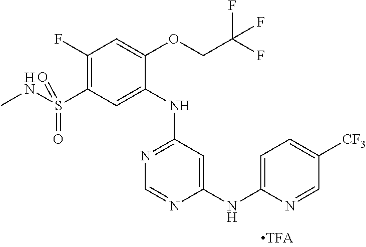 | 5-(trifluoromethyl)-2- pyridinamine |

The following compound was prepared with procedures analogous to that described in Example 318 using 3-[(6-chloro-4-pyrimidinyl)amino]-N-methyl-4-[(2,2,2-trifluoroethyl)oxy]benzenesulfonamide as either the free base or HCl salt, and the specified amine:

| Ex. | Name | Structure | Amine |
|---|---|---|---|
| 320 | 3-({6-[(5-fluoro-2- pyridinyl)amino]-4- pyrimidinyl}amino)-N- methyl-4-[(2,2,2- trifluoroethyl)oxy]benzene- sulfonamide trifluoroacetate | 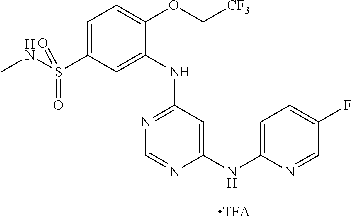 | 5-fluoro-2-pyridinamine |

The following compound was prepared with procedures analogous to that described in Example 318 using 3-[(6-chloro-4-pyrimidinyl)amino]-4-(ethylsulfonyl)-N-methylbenzenesulfonamide as either the free base or HCl salt, and the specified amine:

| Ex. | Name | Structure | Amine |
|---|---|---|---|
| 321 | 3-({6-[(5-chloro-2- pyridinyl)amino]-4- pyrimidinyl}amino)-4- (ethylsulfonyl)-N- methylbenzenesulfonamide trifluoroacetate | 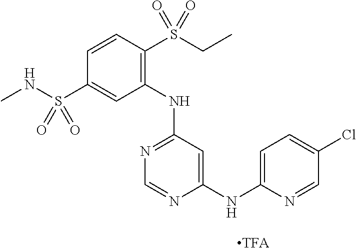 | 5-chloro-2-pyridinamine |
| 322 | 4-(ethylsulfonyl)-N-methyl-3- [(6-{[5-(trifluoromethyl)-2- pyridinyl]amino}-4- pyrimidinyl)amino]benzene- sulfonamide trifluoroacetate | 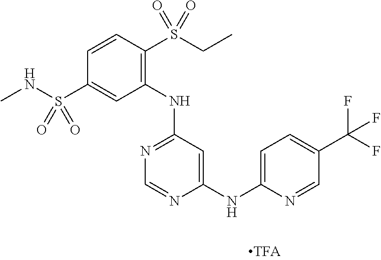 | 5-(trifluoromethyl)-2- pyridinamine |

The following compound was prepared with procedures analogous to that described in Example 318 using 3-[(6-chloro-4-pyrimidinyl)amino]-N-methyl-4-(methylsulfonyl)benzenesulfonamide as either the free base or HCl salt, and the specified amine:

| Ex. | Name | Structure | Amine |
|---|---|---|---|
| 323 | 3-({6-[(5-cyano-2- pyridinyl)amino]-4- pyrimidinyl}amino)-N- methyl-4- (methylsulfonyl)benzene- sulfonamide trifluoroacetate | 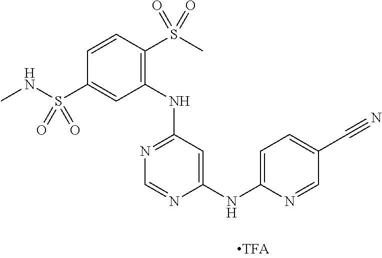 | 6-amino-3- pyridinecarbonitrile |

The following compound was prepared with procedures analogous to that described in Example 318 using 3-[(6-chloro-4-pyrimidinyl)amino]-N-methyl-4-[(2,2,2-trifluoro-1-methylethyl)oxy]benzenesulfonamide as either the free base or HCl salt, and the specified amine:

| Ex. | Name | Structure | Amine |
|---|---|---|---|
| 324 | 3-({6-[(5-cyano-2- pyridinyl)amino]-4- pyrimidinyl}amino)-N- methyl-4-[(2,2,2-trifluoro-1- methylethyl)oxy]benzene- sulfonamide trifluoroacetate | 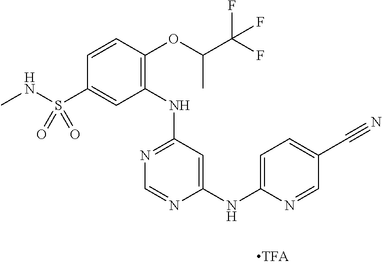 | 6-amino-3- pyridinecarbonitrile |

### Example 325

### 2-{[6-({3-[(methylamino)sulfonyl]phenyl}amino)-4-pyrimidinyl]amino}-1,3-thiazole-5-carboxylic acid

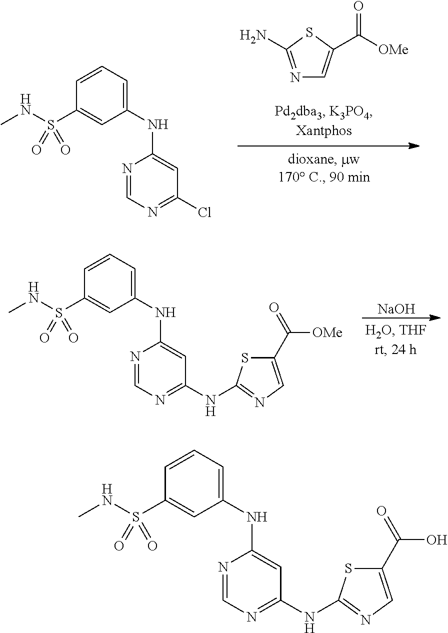

### Step 1. methyl 2-{[6-({3-[(methylamino)sulfonyl]phenyl}amino)-4-pyrimidinyl]amino}-1,3-thiazole-5-carboxylate

A mixture of 3-[(6-chloro-4-pyrimidinyl)amino]-N-methylbenzenesulfonamide (0.150 g, 0.502 mmol), K₃PO₄ (0.213 g, 1.004 mmol), xantphos (0.011 g, 0.020 mmol), Pd₂(dba)₃ (9.20 mg, 0.010 mmol), and methyl 2-amino-1,3-thiazole-5-carboxylate (0.079 g, 0.502 mmol) was heated in a microwave reactor at 170° C. for 90 min. The reaction crude mixture was purified via flash column chromatography (ISCO, 40 g silica column, 0-10% MeOH/CH₂Cl₂) to afford methyl 2-{[6-({3-[(methylamino)sulfonyl]phenyl}amino)-4-pyrimidinyl]amino}-1,3-thiazole-5-carboxylate (0.030 mg, 14%) as an oil. (m/z) 421.0 (M+H⁺)

### Step 2. 2-{[6-({3-[(methylamino)sulfonyl]phenyl}amino)-4-pyrimidinyl]amino}-1,3-thiazole-5-carboxylic acid

A solution of methyl 2-{[6-({3-[(methylamino)sulfonyl]phenyl}amino)-4-pyrimidinyl]amino}-1,3-thiazole-5-carboxylate (0.030 g, 0.071 mmol) in THF (6 mL) and water (2 mL) was treated with NaOH (1 mL, 2.0 mmol) at rt for 24 h. The solvent was removed in vacuo and the residue treated with HCl (1 mL, 2.0 mmol). Collection of the yellow precipitate by filtration followed by lyophilization afforded 2-{[6-({3-[(methylamino)sulfonyl]phenyl}amino)-4-pyrimidinyl]amino}-1,3-thiazole-5-carboxylic acid (0.019 g, 62%).

The following compound was prepared with a procedure analogous to that described in Example 325 using the indicated aniline:

| Ex. | Name | Structure | Aniline |
|---|---|---|---|
| 326 | (2-{[6-({3- [(methylamino)sulfonyl]phenyl} amino)-4-pyrimidinyl]amino}- 1,3-thiazol-4-yl)acetic acid | 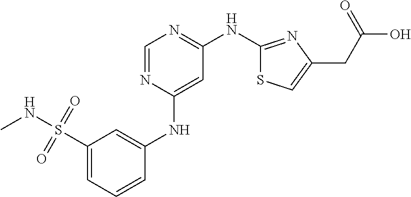 | methyl (2-amino-1,3- thiazol-4-yl)acetate |

### Example 327

### 1-{6-[(4-chlorophenyl)amino]-4-pyrimidinyl}-N-methyl-1H-indole-6-sulfonamide trifluoroacetate

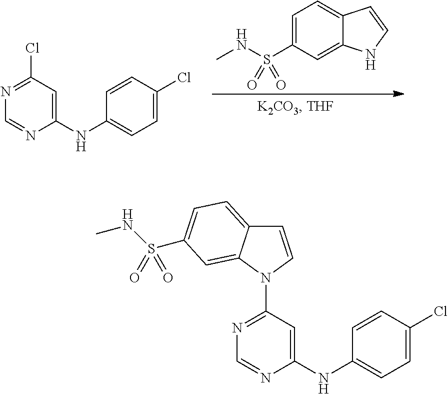

A mixture of N-methyl-1H-indole-6-sulfonamide (230 mg, 1.094 mmol), 6-chloro-N-(4-chlorophenyl)-4-pyrimidinamine (263 mg, 1.094 mmol) in THF was heated in the microwave for 60 min at 150° C. The reaction was filtered and the filtrate concentrated. The residue was dissolved in NMP and purified by mass directed autoprep (Waters, Sunfire prep C18 OBD, 30×150 mm, (40-90% CH₃CN+0.1% TFA/water+0.1% TFA) Concentration of the appropriate fractions yielded 1-{6-[(4-chlorophenyl)amino]-4-pyrimidinyl}-N-methyl-1H-indole-6-sulfonamide trifluoroacetate (63 mg, 5.7%) as a brown solid.

### Example 328

### 3-{6-[(4-chlorophenyl)amino]-4-pyrimidinyl}-N-methyl-2-oxo-2,3-dihydro-1H-benzimidazole-5-sulfonamide trifluoroacetate

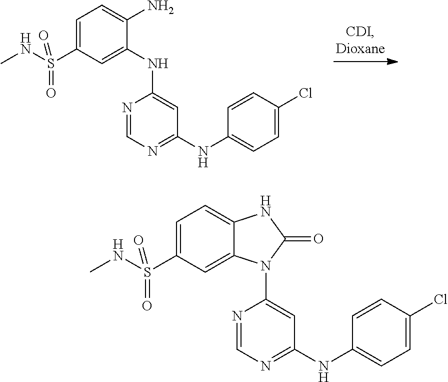

A mixture of 4-amino-3-({6-[(4-chlorophenyl)amino]-4-pyrimidinyl}amino)-N-methylbenzenesulfonamide (400 mg, 0.494 mmol) and carbonyl diimidazole (136 mg, 0.840 mmol) in 1,4-dioxane (1976 μl) was stirred at rt for 5 h then 12 h at 50° C. LCMS analysis of the reaction mixture showed incomplete reaction. The reaction was concentrated and the residue partitioned between CH₂Cl₂ and 2N HCl. The organic layers were concentrated and the residue was dissolved in 1,4-dioxane (2 mL), treated with carbonyl diimidazole (120 mg, 0.741 mmol) and heated in the microwave at 100° C. for a total of 25 min. The reaction mixture was concentrated, the residue was dissolved in NMP, filtered and purified by mass directed autoprep (Waters, Sunfire prep C18 OBD, 30×150 mm, (30-70% CH₃CN+0.1% TFA/water+0.1% TFA) Concentration of the appropriate fractions yielded 3-{6-[(4-chlorophenyl)amino]-4-pyrimidinyl}-N-methyl-2-oxo-2,3-dihydro-1H-benzimidazole-5-sulfonamide trifluoroacetate (12.2 mg, 4.1%) as a solid.

### Example 329

### 3-{[6-({3-[6-(dimethylamino)-3-pyridinyl]phenyl}amino)-4-pyrimidinyl]amino}-N-methylbenzenesulfonamide

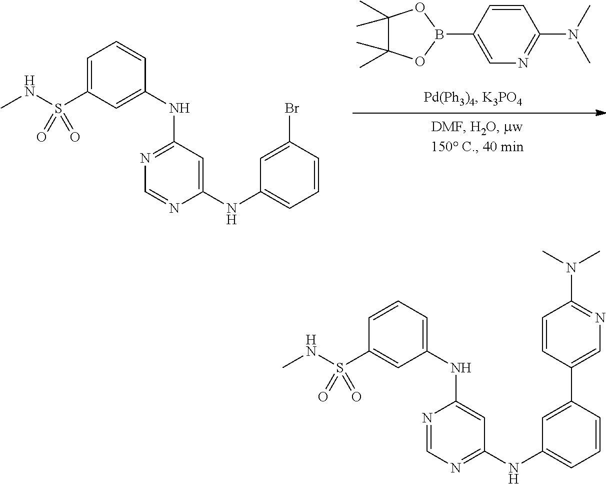

A mixture of 3-({6-[(3-bromophenyl)amino]-4-pyrimidinyl}amino)-N-methylbenzenesulfonamide (0.500 g, 1.15 mmol), N,N-dimethyl-5-(4,4,5,5-tetramethyl-1,3,2-dioxaborolan-2-yl)-2-pyridinamine (0.429, 1.732), K₃PO₄ (1.23 g, 4.6 mmol), and Pd(Ph₃)₄ (0.133 g, 0.115 mmol) was heated in DMF (6 mL) and water (0.6 mL) in a microwave reactor for 40 min at 150° C. The reaction mixture was then cooled, diluted with 10% MeOH/CH₂Cl₂ (50 mL), filtered, and concentrated. The crude material was then purified via flash column chromatography (40 g silica column, 20:1:0.1 CH₂Cl₂:MeOH:Et₃N) to give 3-{[6-({3-[6-(dimethylamino)-3-pyridinyl]phenyl}amino)-4-pyrimidinyl]amino}-N-methylbenzenesulfonamide (0.350 g) in 85% purity. This material was then purified via HPLC (Gilson, PRC-ODS 20×250 mm column, 55-70% CH₃CN/H₂O with 0.01% NH₄HCO₃) to afford 3-{[6-({3-[6-(dimethylamino)-3-pyridinyl]phenyl}amino)-4-pyrimidinyl]amino}-N-methylbenzenesulfonamide in >99% purity (0.150 g, 35%) as a white solid.

The following compounds were prepared with procedures analogous to that described in Example 329 using 3-({6-[(3-bromo-5-methylphenyl)amino]-4-pyrimidinyl}amino)-N-methylbenzenesulfonamide as the free base, TFA, or HCl salt and the specified boronic acid:

| Ex. | Name | Structure | Boronate |
|---|---|---|---|
| 330 | N-methyl-3-({6-[(5-methyl- 3-biphenylyl)amino]-4- pyrimidinyl}amino)benzene sulfonamide trifluoroacetate | 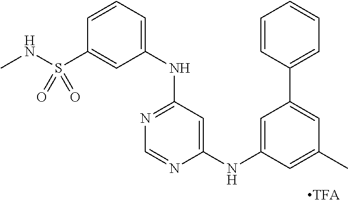 | Phenyl boronic acid |
| 331 | N-methyl-3-[(6-{[3-methyl-5- (3-pyridinyl)phenyl]amino}- 4-pyrimidinyl)amino]- benzenesulfonamide trifluoroacetate | 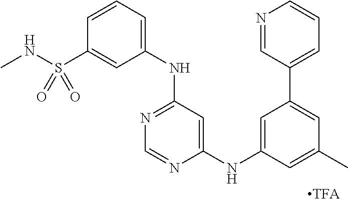 | 3-pyridinylboronic acid |

The following compounds were prepared with procedures analogous to that described in Example 329 using 3-({6-[(3-bromophenyl)amino]-4-pyrimidinyl}amino)-N-methylbenzenesulfonamide as the free base, TFA, or HCl salt and the specified boronate:

| Ex. | Name | Structure | Boronate |
|---|---|---|---|
| 332 | 3-[(6-{[3′-(dimethylamino)- 3-biphenylyl]amino}-4- pyrimidinyl)amino]-N- methylbenzenesulfonamide | 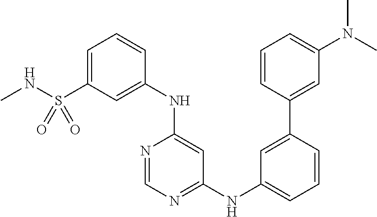 | [3- (dimethylamino)phenyl] boronic acid |
| 333 | N-methyl-3-[(6-{[4′-(4- morpholinyl)-3- biphenylyl]amino}-4- pyrimidinyl)amino]- benzenesulfonamide | 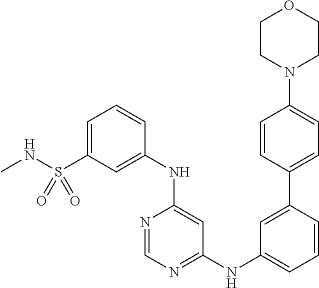 | [4-(4- morpholinyl)phenyl] boronic acid |
| 334 | N-methyl-3-{[6-({3-[6- (methyloxy)-3- pyridinyl]phenyl}amino)-4- pyrimidinyl]amino}- benzenesulfonamide | 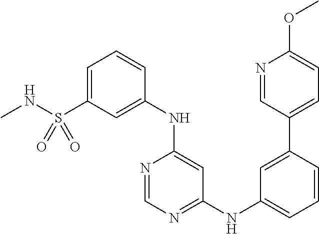 | [6-(methyloxy)-3- pyridinyl]boronic acid |
| 335 | 3′-{[6-({3- [(methylamino)sulfonyl] phenyl}amino)-4- pyrimidinyl]amino}-4- biphenylcarboxamide | 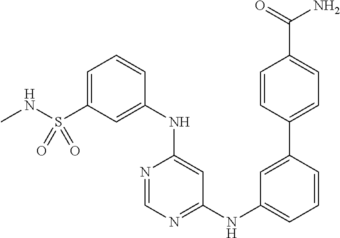 | [4- (aminocarbonyl)phenyl] boronic acid |
| 336 | N-methyl-3-{[6-({3-[5- (methyloxy)-3- pyridinyl]phenyl}amino)-4- pyrimidinyl]amino}- benzenesulfonamide | 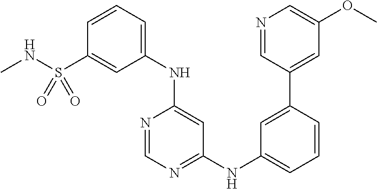 | [5-(methyloxy)-3- pyridinyl]boronic acid |
| 337 | 3′-{[6-({3- [(methylamino)sulfonyl] phenyl}amino)-4- pyrimidinyl]amino}-3- biphenylcarboxamide | 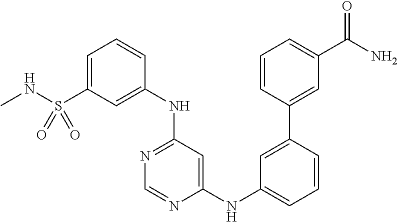 | [3-(aminocarbonyl)phenyl] boronic acid |
| 338 | N-methyl-3-{[6-({3′- [(methylsulfonyl)amino]-3- biphenylyl}amino)-4- pyrimidinyl]amino} benzenesulfonamide | 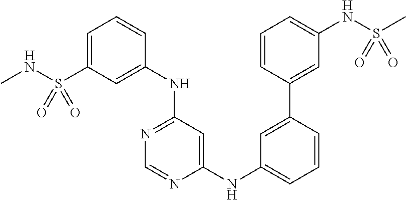 | {3- [(methylsulfonyl)amino]phenyl} boronic acid |
| 339 | 3-[(6-{[4′-(dimethylamino)- 3-biphenylyl]amino}-4- pyrimidinyl)amino]-N- methylbenzenesulfonamide | 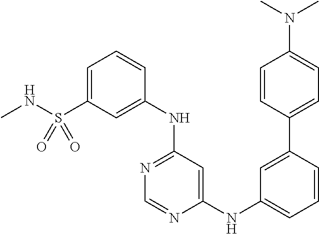 | [4- (dimethylamino)phenyl] boronic acid |
| 340 | N-methyl-3-{[6-({3-[4- (methyloxy)-3- pyridinyl]phenyl}amino)-4- pyrimidinyl]amino}- benzenesulfonamide | 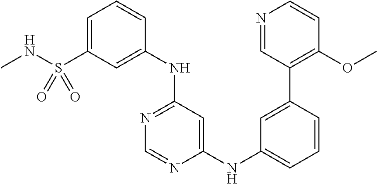 | [4-(methyloxy)-3- pyridinyl]boronic acid |
| 341 | N-(3′-{[6-({3- [(methylamino)sulfonyl] phenyl}amino)-4- pyrimidinyl]amino}-4- biphenylyl)acetamide | 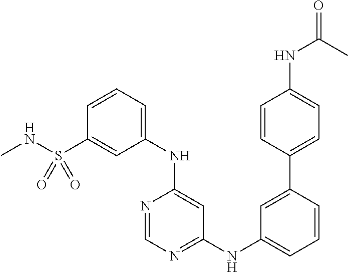 | [4-(acetylamino)phenyl] boronic acid |
| 342 | N-methyl-3-{[6-({4′- [(methylsulfonyl)amino]-3- biphenylyl}amino)-4- pyrimidinyl]amino}- benzenesulfonamide | 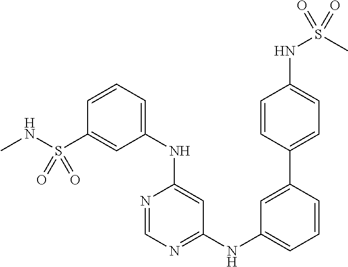 | {4- [(methylsulfonyl)amino]phenyl} boronic acid |
| 343 | N-(3′-{[6-({3- [(methylamino)sulfonyl] phenyl}amino)-4- pyrimidinyl]amino}-3- biphenylyl)acetamicle | 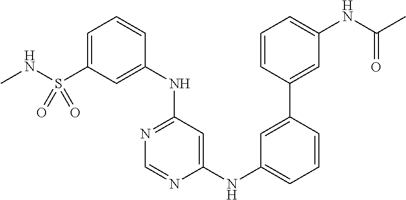 | [3-(acetylamino)phenyl] boronic acid |
| 344 | N-methyl-3′-{[6-({3- [(methylamino)sulfonyl] phenyl}amino)-4- pyrimidinyl]amino}-4- biphenylsulfonamide | 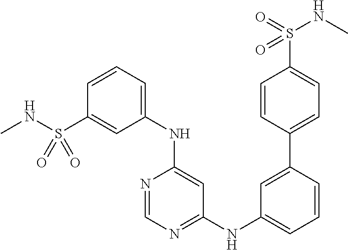 | N-methyl-4-(4,4,5,5- tetramethyl-1,3,2- dioxaborolan-2- yl)benzenesulfonamide |
| 345 | N-methyl-3′-{[6-({3- [(methylamino)sulfonyl] phenyl}amino)-4- pyrimidinyl]amino}-3- biphenylsulfonamide | 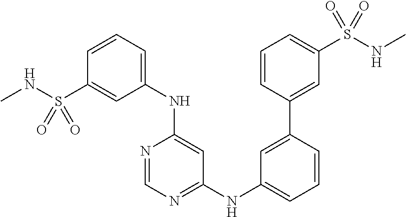 | N-methyl-3-(4,4,5,5- tetramethyl-1,3,2- dioxaborolan-2- yl)benzenesulfonamide |

The following compounds were prepared with procedures analogous to that described in Example 329 using 3-({6-[(3-bromo-4-chlorophenyl)amino]-4-pyrimidinyl}amino)-N-methylbenzenesulfonamide as the free base, TFA, or HCl salt and the specified boronate:

| Ex. | Name | Structure | Boronate |
|---|---|---|---|
| 346 | 3-[(6-{[4-chloro-3-(3- pyridinyl)phenyl]amino}-4- pyrimidinyl)amino]-N- methylbenzenesulfonamide | 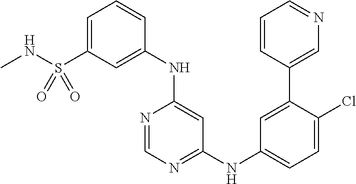 | 3-pyridinylboronic acid |
| 347 | 2′-chloro-5′-{[6-({3- [(methylamino)sulfonyl] phenyl}amino)-4- pyrimidinyl]amino}-3- biphenylcarboxamide | 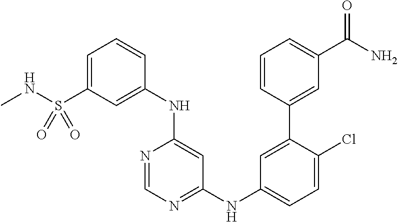 | [3- (aminocarbonyl)phenyl] boronic acid |
| 348 | 3-[(6-{[6-chloro-3′-(4- morpholinyl)-3- biphenylyl]amino}-4- pyrimidinyl)amino]-N- methylbenzenesulfonamide | 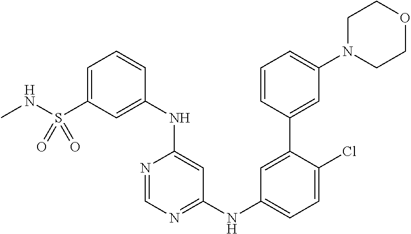 | 4-[3-(4,4,5,5-tetramethyl- 1,3,2-dioxaborolan-2- yl)phenyl]morpholine |

### Example 349

### 4-{[6-({3-[(methylamino)sulfonyl]phenyl}amino)-4-pyrimidinyl]amino}benzoic acid

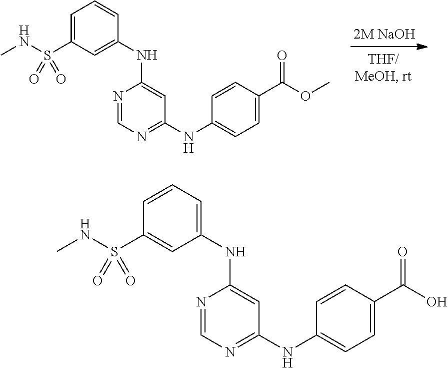

A suspension of methyl 4-{[6-({3-[(methylamino)sulfonyl]phenyl}amino)-4-pyrimidinyl]amino}benzoate (0.070 g, 0.169 mmol), in MeOH (0.212 mL) and THF (0.212 mL) was treated with 2 M NaOH (0.339 mL, 0.677 mmol). After about 15 min, a clear solution was observed. After 1 h additional 2 M NaOH (0.339 mL, 0.677 mmol) was added and the reaction was stirred at rt overnight.

The reaction was acidified to pH 4, the solvent removed in vacuo, and the residue partitioned between CH₂Cl₂ and water. The organic layer was collected via hydrophobic frit. A solid was noted at the interface which was collected by filtration and then dissolved in MeOH and combined with the CH₂Cl₂ extracts. Concentration then afforded 4-{[6-({3-[(methylamino)sulfonyl]phenyl}amino)-4-pyrimidinyl]amino}benzoic acid (0.044 g, 62%) as an off-white solid.

The following carboxylic acid was prepared with a procedure analogous to that described in Example 349 using the specified ester starting material:

| Ex. | Name | Structure | Ester |
|---|---|---|---|
| 350 | [(3-{[6-({3- [(methylamino)sulfonyl]phenyl} amino)-4- pyrimidinyl]amino}phenyl)oxy]- acetic acid | 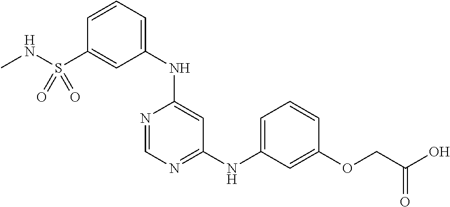 | 1-methylethyl [(3-{[6-({3- [(methylamino)sulfonyl] phenyl}amino)-4- pyrimidinyl]amino}phenyl) oxy]acetate |

### Example 351

### N,N-dimethyl-4-{[6-({3-[(methylamino)sulfonyl]phenyl}amino)-4-pyrimidinyl]amino}benzamide

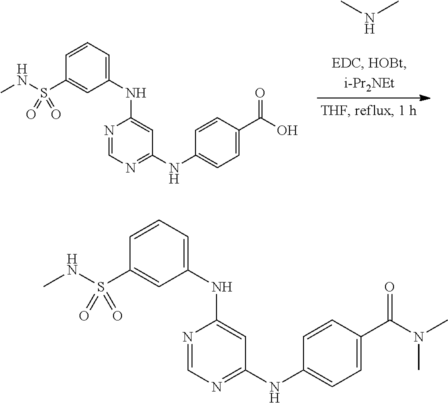

To a solution of 4-{[6-({3-[(methylamino)sulfonyl]phenyl}amino)-4-pyrimidinyl]amino}benzoic acid (0.200 g, 0.50 mmol), dimethylamine (0.027 g, 0.60 mmol), and i-Pr₂NEt (0.223 g, 1.72 mmol) in THF (15 mL), EDC (0.191 g, 1.0 mmol) and HOBT (0.135 g, 1.0 mmol) were added. The resulting mixture was heated to reflux for 1 h. The solvent was removed, the residue diluted with water and filtered to afford N,N-dimethyl-4-{[6-({3-[(methylamino)sulfonyl]phenyl}amino)-4-pyrimidinyl]amino}benzamide (0.140, 65%) as a white solid.

The following compounds were prepared with [(3-{[6-({3-[(methylamino)sulfonyl]phenyl}amino)-4-pyrimidinyl]amino}phenyl)oxy]acetic acid and the specified amine:

| Ex. | Name | Structure | Amine |
|---|---|---|---|
| 352 | N,N-dimethyl-2-[(3-{[6-({3- [(methylamino)sulfonyl]phenyl} amino)-4- pyrimidinyl]amino}phenyl)oxy] acetamide trifluoroacetate | 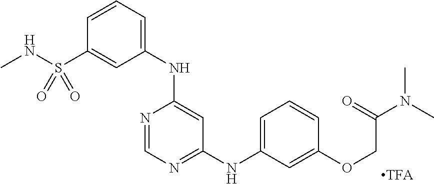 | dimethylamine |

The following compounds were prepared with procedures analogous to that described in Example 351 using 4-{[6-({3-[(methylamino)sulfonyl]phenyl}amino)-4-pyrimidinyl]amino}benzoic acid and the specified amine:

| Ex. | Name | Structure | Amine |
|---|---|---|---|
| 353 | N-(2-hydroxyethyl)-4-{[6-({3- [(methylamino)sulfonyl]phenyl} amino)-4- pyrimidinyl]amino}benzamide | 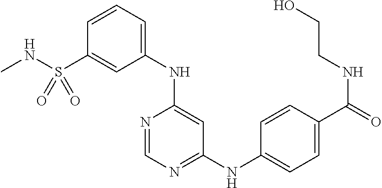 | 2-aminoethanol |
| 354 | N-methyl-3-{[6-({4-[(4-methyl- 1-piperazinyl)carbonyl]phenyl} amino)-4- pyrimidinyl]amino}benzene- sulfonamide | 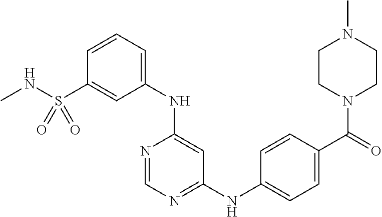 | 1-methylpiperazine |
| 355 | 4-{[6-({3- [(methylamino)sulfonyl]phenyl} amino)-4-pyrimidinyl]amino}-N- (1-methyl-4- piperidinyl)benzamide | 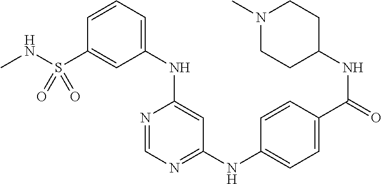 | 1-methyl-4-piperidinamine |
| 356 | N-methyl-3-[(6-{[4-(1- piperazinylcarbonyl)phenyl] amino}-4- pyrimidinyl)amino]benzene- sulfonamide | 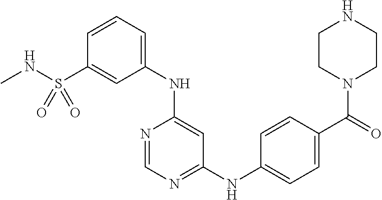 | piperazine |
| 357 | N-methyl-3-[(6-{[4-({4-[2- (methyloxy)ethyl]-1- piperazinyl}carbonyl)phenyl] amino}-4- pyrimidinyl)amino]benzene- sulfonamide | 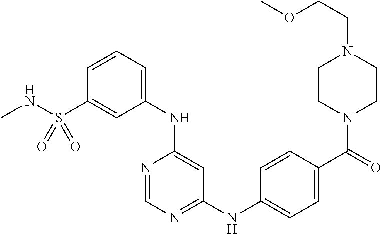 | 1-[2- (methyloxy)ethyl]piperazine |
| 358 | 4-{[6-({3- [(methylamino)sulfonyl]phenyl} amino)-4-pyrimidinyl]amino}-N- [2-(methyloxy)ethyl]benzamide | 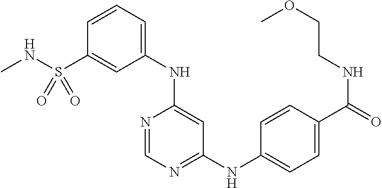 | 2-(methyloxy)ethanamine |
| 359 | 4-{[6-({3- [(methylamino)sulfonyl]phenyl} amino)-4-pyrimidinyl]amino}-N- [3- (methyloxy)propyl]benzamide | 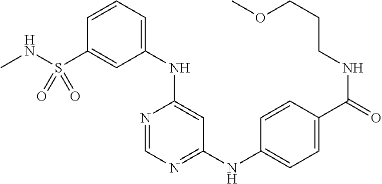 | 3-(methyloxy)-1-propanamine |
| 360 | N-[2-(dimethylamino)ethyl]-4- {[6-({3- [(methylamino)sulfonyl]phenyl} amino)-4- pyrimidinyl]amino}benzamide | 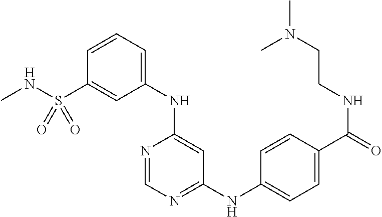 | N,N-dimethyl-1,2- ethanediamine |
| 361 | N,N-diethyl-4-{[6-({3- [(methylamino)sulfonyl]phenyl} amino)-4- pyrimidinyl]amino}benzamide | 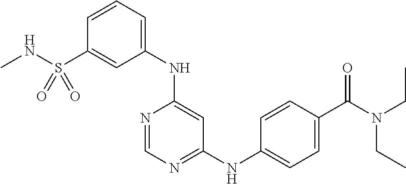 | diethylamine |
| 362 | N-methyl-3-[(6-{[4-(1- pyrrolidinylcarbonyl)phenyl] amino}-4- pyrimidinyl)amino]benzene- sulfonamide | 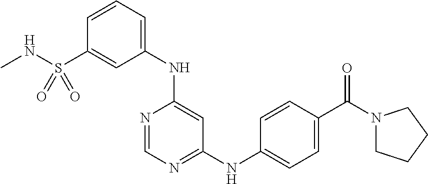 | pyrollidine |
| 363 | 3-({6-[(4-{[(3S)-3- (dimethylamino)-1- pyrrolidinyl]carbonyl}phenyl) amino]-4-pyrimidinyl}amino)-N- methylbenzenesulfonamide | 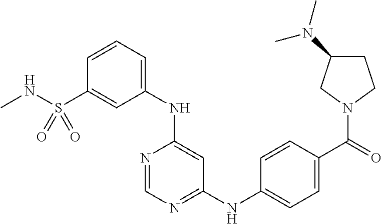 | (3S)-N,N-dimethyl-3- pyrrolidinamine |
| 364 | N-methyl-3-{[6-({4-[(4- methylhexahydro-1H-1,4- diazepin-1- yl)carbonyl]phenyl}amino)-4- pyrimidinyl]amino}benzene- sulfonamide | 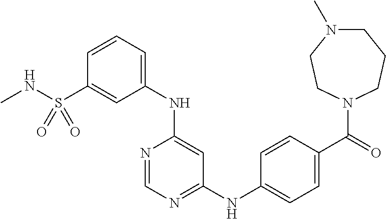 | 1-methylhexahydro-1H- 1,4-diazepine |
| 365 | N-methyl-3-[(6-{[4-(4- thiomorpholinylcarbonyl)phenyl] amino}-4- pyrimidinyl)amino]benzene- sulfonamide | 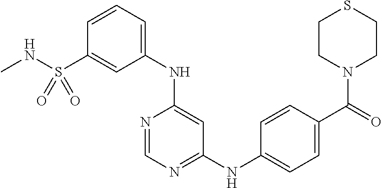 | thiomorpholine |
| 366 | 3-{[6-({4-[(4,4-difluoro-1- piperidinyl)carbonyl]phenyl} amino)-4-pyrimidinyl]amino}-N- methylbenzenesulfonamide | 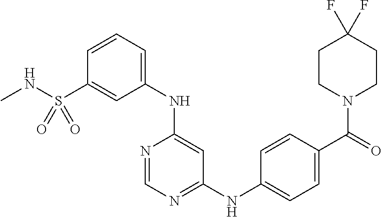 | 4,4-difluoropiperidine |
| 367 | 3-({6-[(4-{[(3R)-3- (dimethylamino)-1- pyrrolidinyl]carbonyl}phenyl) amino]-4-pyrimidinyl}amino)-N- methylbenzenesulfonamide | 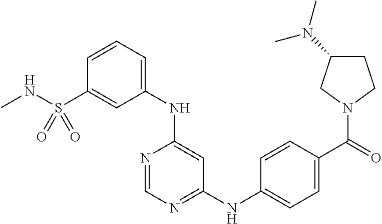 | (3R)-N,N-dimethyl-3- pyrrolidinamine |
| 368 | N-[2-(dimethylamino)ethyl]-N- methyl-4-{[6-({3- [(methylamino)sulfonyl]phenyl} amino)-4- pyrimidinyl]amino}benzamide | 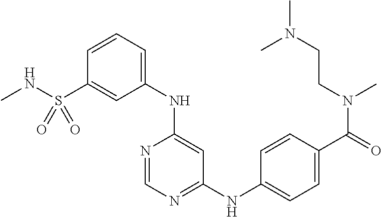 | [2- (dimethylamino)ethyl] methylamine |

The following compound was prepared with procedures analogous to that described in Example 351 using the 4-[(6-{[5-[(methylamino)sulfonyl]-2-(methylthio)phenyl]amino}-4-pyrimidinyl)amino]benzoic acid and the appropriate amine:

| Ex. | Name | Structure | Amine |
|---|---|---|---|
| 369 | N-[2-(dimethylamino)ethyl]-N- methyl-4-[(6-{[5- [(methylamino)sulfonyl]-2- (methylthio)phenyl]amino}-4- pyrimidinyl)amino]benzamide trifluoroacetate | 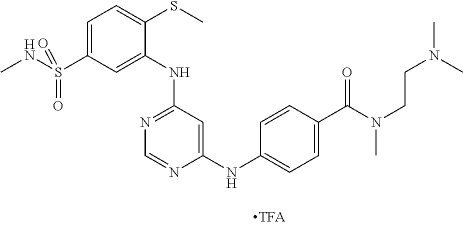 | [2- (dimethylamino)ethyl] methylamine |

### Example 370

### N-[(4-{[6-({3-[(methylamino)sulfonyl]phenyl}amino)-4-pyrimidinyl]amino}phenyl)carbonyl]glycine

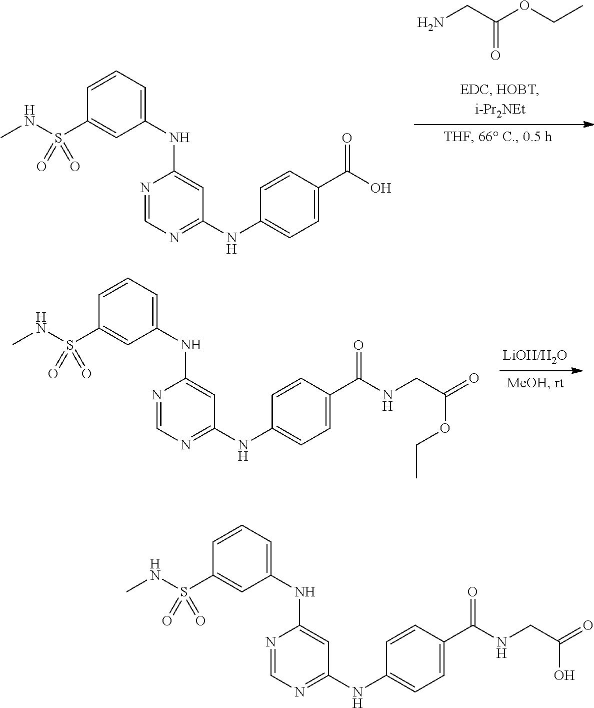

### Step 1. ethyl N-[(4-{[6-({3-[(methylamino)sulfonyl]phenyl}amino)-4-pyrimidinyl]amino}phenyl)carbonyl]glycinate

To a solution of 4-{[6-({3-[(methylamino)sulfonyl]phenyl}amino)-4-pyrimidinyl]amino}benzoic acid (0.200 g, 0.50 mmol), ethyl glycinate (0.099 g, 0.75 mmol), and i-Pr₂NEt (0.260 g, 2.00 mmol) in THF (50 mL), EDC (0.196 g, 1.0 mmol) and HOBT (0.135 g, 1.0 mmol) were added. The resulting mixture was heated to reflux for 0.5 h. The solvent was removed, the residue diluted with water and filtered to afford ethyl N-[(4-{[6-({3-[(methylamino)sulfonyl]phenyl}amino)-4-pyrimidinyl]amino}phenyl)carbonyl]glycinate (0.200 g, 83%) as a white solid.

### Step 2. N-[(4-{[6-({3-[(methylamino)sulfonyl]phenyl}amino)-4-pyrimidinyl]amino}phenyl) carbonyl]glycine

A mixture of N-[(4-{[6-({3-[(methylamino)sulfonyl]phenyl}amino)-4-pyrimidinyl]amino}phenyl)carbonyl]glycinate (0.200 g, 0.414 mmol) and LiOH (6 mL of a 1 M solution in water, 6.0 mmol) in MeOH (20 mL) was stirred at rt. When the ester had been consumed, the MeOH was removed in vacuo and the residue acidified to pH 5. A white solid then formed which was removed via filtration to afford N-[(4-{[6-({3-[(methylamino)sulfonyl]phenyl}amino)-4-pyrimidinyl]amino}phenyl)carbonyl]glycine (0.040 g, 21%).

### Example 371

### N-methyl-3-[(6-{[3-(6-oxo-1,6-dihydro-3-pyridinyl)phenyl]amino}-4-pyrimidinyl)amino]benzenesulfonamide

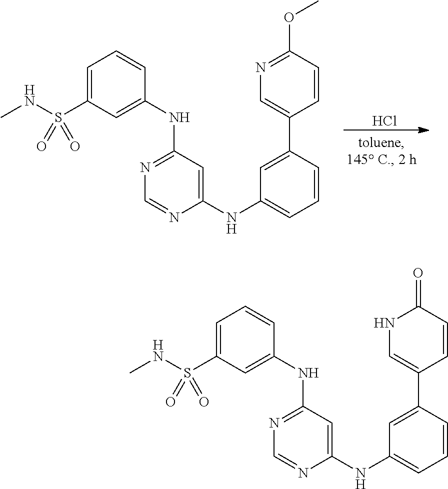

To a solution of N-methyl-3-{[6-({3-[6-(methyloxy)-3-pyridinyl]phenyl}amino)-4-pyrimidinyl]amino}benzenesulfonamide (0.200 g, 0.44 mmol) in toluene (4 mL), HCl (2 mL of a 35% solution) was added. The reaction mixture was then heated to 145° C. in a sealed tube for 2 h. The crude material was then purified via preparatory HPLC (250×19 mm column, 35-60% 0.01% NH₄HCO₃ in H₂O/CH₃CN) to afford N-methyl-3-[(6-{[3-(6-oxo-1,6-dihydro-3-pyridinyl)phenyl]amino}-4-pyrimidinyl)amino]benzenesulfonamide (0.128 g, 65%) as a yellow solid.

### Example 372

### 3-({6-[(3-hydroxyphenyl)amino]-4-pyrimidinyl}amino)-N-methylbenzenesulfonamide trifluoroacetate

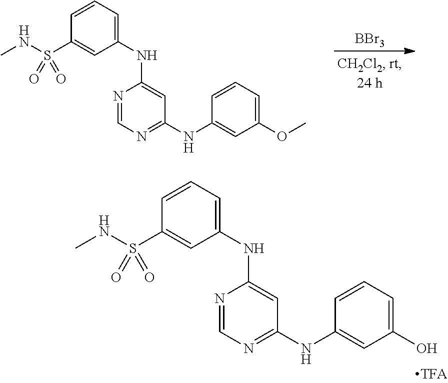

A solution of N-methyl-3-[(6-{[3-(methyloxy)phenyl]amino}-4-pyrimidinyl)amino]benzenesulfonamide (0.040 g, 0.104 mmol) in CH₂Cl₂ (15 mL) was treated with BBr₃ (0.059 mL, 0.623 mmol) at rt for 24 h. The reaction mixture was quenched slowly with a satd. NH₄Cl solution (1 mL) and then partitioned between 100 mL EtOAc and 20 mL of brine. The organic layer was separated, dried over MgSO₄, filtered and concentrated in vacuo. The crude material was then purified through reverse phase HPLC (Sunfire C-18 prep column, 30×50 mm column, 10-50% CH₃CN/water with 0.1% TFA over 14 min). The appropriate fractions were then concentrated and lyophilized to afford 3-({6-[(3-hydroxyphenyl)amino]-4-pyrimidinyl}amino)-N-methylbenzenesulfonamide trifluoroacetate (0.019 g, 36%) as a white solid.

### Example 373

### N-methyl-4-(methylsulfonyl)-3-[(6-{[4-(trifluoromethyl)phenyl]amino}-4-pyrimidinyl)amino]benzenesulfonamide

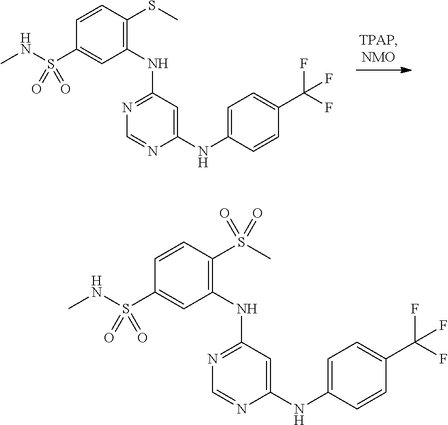

A mixture of N-methyl-4-(methylthio)-3-[(6-{[4-(trifluoromethyl)phenyl]amino}-4-pyrimidinyl)amino]benzenesulfonamide (100 mg, 0.213 mmol), NMO (74.9 mg, 0.639 mmol), TPAP (3.74 mg, 10.65 μmol) and 4 Å powdered molecular sieves (0.213 mmol) in CH₃CN (0.532 mL) was stirred at 40° C. for 3 h. An additional portion of TPAP (3.74 mg, 10.65 μmol) was added and the reaction was stirred at 40° C. for an additional 20 hrs before being cooled to rt and loaded onto a silica solid phase extraction column (2 g, washed with CH₂Cl₂, Et₂O, EtOAc, acetone). Concentration of the appropriate fractions yielded the crude product, which was further purified by ion exchange column (SCX, 2 g, washed with MeOH and eluted with 10% 2M ammonia in MeOH in CH₂Cl₂). Concentration of the appropriate fractions yielded a solid which was triturated with CH₂Cl₂ to afford N-methyl-4-(methylsulfonyl)-3-[(6-{[4-(trifluoromethyl)phenyl]amino}-4-pyrimidinyl)amino]benzenesulfonamide (5 mg, 3%) as a white solid.

### Example 374

### 3-({6-[(4-chlorophenyl)amino]-4-pyrimidinyl}amino)-N-methyl-4-(methylsulfonyl)benzenesulfonamide trifluoroacetate

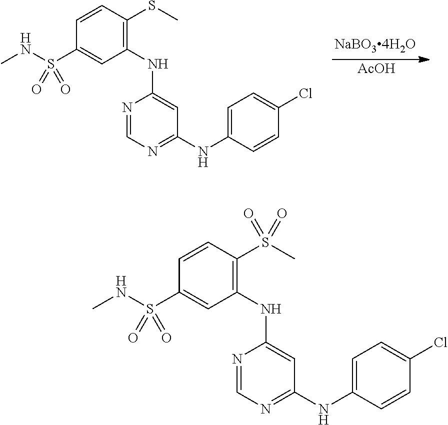

A mixture of 3-({6-[(4-chlorophenyl)amino]-4-pyrimidinyl}amino)-N-methyl-4-(methylthio)benzenesulfonamide (100 mg, 0.229 mmol) and sodium perborate tetrahydrate (141 mg, 0.918 mmol) in AcOH (0.184 mL) was heated at 50° C. overnight. The reaction was then diluted by the addition of water and extracted with CH₂Cl₂. The organic was collected by hydrophobic frit and concentrated to give a orange solid, 96 mg. This solid was then purified by mass directed autoprep (Waters, Sunfire prep C18 OBD, 30×150 mm, (30-70% CH₃CN+0.1% TFA/water+0.1% TFA). Concentration of the appropriate fractions yielded 3-({6-[(4-chlorophenyl)amino]-4-pyrimidinyl}amino)-N-methyl-4-(methylsulfonyl)benzenesulfonamide trifluoroacetate (52 mg, 32%) as a peach coloured solid.

The following examples were prepared with procedures analogous to that described in Example 374 using the specified sulphide:

| Ex. | Name | Structure | Sulphide |
|---|---|---|---|
| 375 | 3-(6-(4- chlorophenylamino)pyrimidin- 4-ylamino)-4-(isobutylsulfonyl)- N-methylbenzenesulfonamide trifluoroacetate | 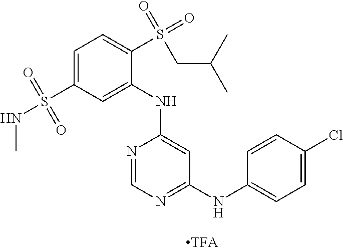 | 3-({6-[(4- chlorophenyl)amino]- 4-pyrimidinyl}amino)- N-methyl-4-[(2- methylpropyl)thio] benzenesulfonamide |
| 376 | 3-(6-(4- chlorophenylamino)pyrimidin- 4-ylamino)-4-(ethylsulfonyl)-N- methylbenzenesulfonamide trifluoroacetate | 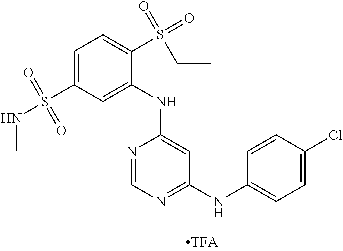 | 3-({6-[(4- chlorophenyl)amino]- 4-pyrimidinyl}amino)- 4-(ethylthio)-N- methylbenzene- sulfonamide |

### Examples 377 & 378

### 3-({6-[(4-chlorophenyl)amino]-4-pyrimidinyl}amino)-N-methyl-4-[(2,2,2-trifluoro-1-methylethyl)oxy]benzenesulfonamide (enantiomer 1)

### 3-({6-[(4-chlorophenyl)amino]-4-pyrimidinyl}amino)-N-methyl-4-[(2,2,2-trifluoro-1-methylethyl)oxy]benzenesulfonamide (enantiomer 2)

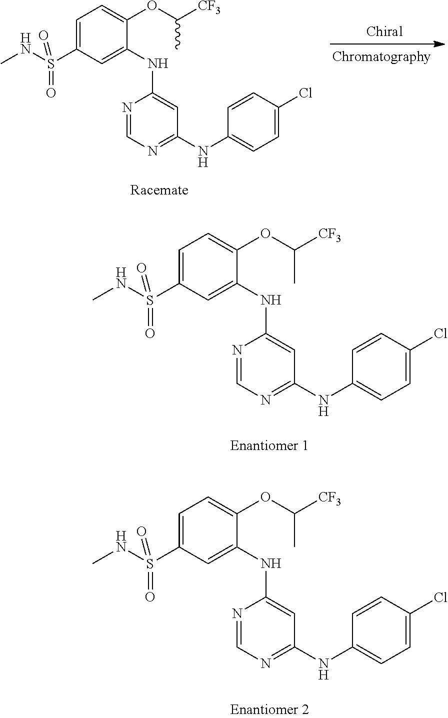

A racemic mixture of 3-({6-[(4-chlorophenyl)amino]-4-pyrimidinyl}amino)-N-methyl-4-[(2,2,2-trifluoro-1-methylethyl)oxy]benzenesulfonamide (475 mg) was subjected to chiral chromatography (Chiralpak AD-H, 60% IPA, 40% hexanes) to provide 3-({6-[(4-chlorophenyl)amino]-4-pyrimidinyl}amino)-N-methyl-4-[(2,2,2-trifluoro-1-methylethyl)oxy]benzenesulfonamide (unassigned enantiomer 1, 20.2 mg) and 3-({6-[(4-chlorophenyl)amino]-4-pyrimidinyl}amino)-N-methyl-4-[(2,2,2-trifluoro-1-methylethyl)oxy]benzenesulfonamide (unassigned enantiomer 2, 20.8 mg)

### Examples 379 & 380

### 3-({6-[(5-chloro-2-pyridinyl)amino]-4-pyrimidinyl}amino)-N-methyl-4-[(2,2,2-trifluoro-1-methylethyl)oxy]benzenesulfonamide (enantiomer 1)

### 3-({6-[(5-chloro-2-pyridinyl)amino]-4-pyrimidinyl}amino)-N-methyl-4-[(2,2,2-trifluoro-1-methylethyl)oxy]benzenesulfonamide (enantiomer 2)

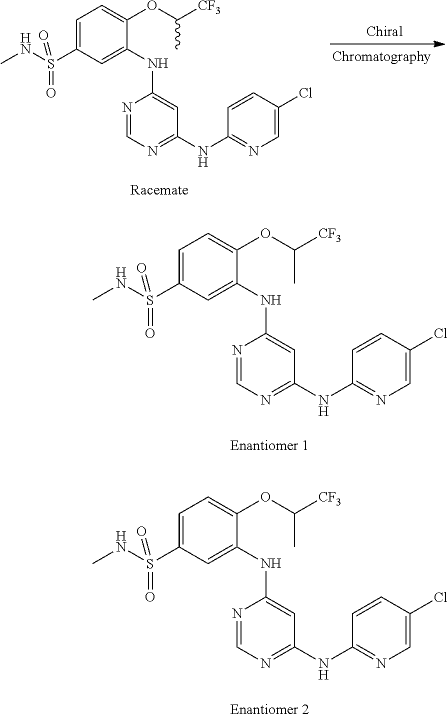

A racemic mixture of 3-({6-[(5-chloro-2-pyridinyl)amino]-4-pyrimidinyl}amino)-N-methyl-4-[(2,2,2-trifluoro-1-methylethyl)oxy]benzenesulfonamide (373 mg) was subjected to chiral chromatography (Chiralpak AD-H, 60% IPA, 40% hexanes with 0.1% DEA ad a modifier) to provide 3-({6-[(5-chloro-2-pyridinyl)amino]-4-pyrimidinyl}amino)-N-methyl-4-[(2,2,2-trifluoro-1-methylethyl)oxy]benzenesulfonamide (unassigned enantiomer 1, 80 mg) & 3-({6-[(5-chloro-2-pyridinyl)amino]-4-pyrimidinyl}amino)-N-methyl-4-[(2,2,2-trifluoro-1-methylethyl)oxy]benzenesulfonamide (unassigned enantiomer 2.39 mg, 85% ee).

### Spectroscopic Data for Examples 1-380:

| | | t_{R} | MS | |
|---|---|---|---|---|
| Ex. | Name | (min) | (m/z) | ¹H NMR |
| 1 | N-methyl-3-({6-[(3- | 1.93^{a} | 370.1 (M + H)⁺ | ¹H NMR (400 MHz, DMSO-d₆) δ ppm |
| | methylphenyl)amino]-4- | | | 9.75 (s, 1H), 9.43 (br. s., 1H), 8.37 (s, |
| | pyrimidinyl}amino)benzenesulfonamide | | | 1H), 8.02-8.11 (m, 1H), 7.87 (dd, J = 1.51, |
| | trifluoroacetate | | | 8.03 Hz, 1H), 7.54 (t, J = 7.91 Hz, |
| | | | | 1H), 7.46 (q, J = 4.85 Hz, 1H), |
| | | | | 7.38 (d, J = 7.78 Hz, 1H), |
| | | | | 7.29-7.35 (m, 2H), 7.20-7.27 (m, 1H), 6.90 (d, |
| | | | | J = 7.28 Hz, 1H), 6.18 (s, 1H), 2.44 (d, |
| | | | | J = 4.77 Hz, 3H), 2.31 (s, 3H) |
| 2 | 3-({6-[(3-chlorophenyl)amino]-4- | 2.17^{a} | 390.1 (M + H)⁺ | ¹H NMR (400 MHz, DMSO-d₆) δ ppm |
| | pyrimidinyl}amino)-N- | | | 9.68 (s, 1H), 9.54 (s, 1H), 8.41 (s, |
| | methylbenzenesulfonamide | | | 1H), 8.09 (t, J = 1.88 Hz, 1H), |
| | trifluoroacetate | | | 7.90-7.94 (m, 1H), 7.88 (t, J = 2.01 Hz, |
| | | | | 1H), 7.53 (t, J = 7.91 Hz, 1H), |
| | | | | 7.41-7.49 (m, 2H), 7.29-7.39 (m, 2H), |
| | | | | 7.03 (dd, J = 1.25, 8.03 Hz, 1H), |
| | | | | 6.21 (s, 1H), 2.45 (d, J = 4.77 Hz, 3H) |
| 3 | N-methyl-3-{[6-(methylamino)-4- | 1.28^{a} | 294.0 (M + H)⁺ | ¹H NMR (400 MHz, DMSO-d₆) δ ppm |
| | pyrimidinyl]amino}benzenesulfonamide | | | 10.61 (br. s., 1H), 8.82 (br. s., 1H), |
| | hydrochloride | | | 8.44 (s, 1H), 7.95 (br. s., 1H), |
| | | | | 7.75 (br. s., 1H), 7.55-7.67 (m, 2H), |
| | | | | 7.52 (d, J = 7.28 Hz, 1H), 6.08 (br. s., 1H), |
| | | | | 2.88 (br. s., 3H), 2.45 (d, J = 4.77 Hz, |
| | | | | 3H) |
| 4 | 3-{[6-(ethylamino)-4- | 1.54^{a} | 308.1 (M + H)⁺ | ¹H NMR (400 MHz, DMSO-d₆) δ ppm |
| | pyrimidinyl]amino}-N- | | | 9.30 (s, 1H), 8.15 (s, 1H), 8.09 (s, |
| | methylbenzenesulfonamide | | | 1H), 7.83-7.88 (m, 1H), 7.47 (t, J = 7.91 Hz, |
| | hydrochloride | | | 1H), 7.40 (q, J = 5.02 Hz, |
| | | | | 1H), 7.28 (d, J = 8.03 Hz, 1H), 6.97 (t, |
| | | | | J = 4.77 Hz, 1H), 5.76 (s, 1H), |
| | | | | 3.16-3.27 (m, 2H), 2.44 (d, J = 5.02 Hz, |
| | | | | 3H), 1.13 (t, J = 7.15 Hz, 3H) |
| 5 | 3,3′-(4,6- | 1.88^{a} | 449.1 (M + H)⁺ | ¹H NMR (400 MHz, DMSO-d₆) δ ppm |
| | pyrimidinediyldiimino)bis(N- | | | 9.75 (s, 2H), 8.41 (s, 1H), 8.08 (s, |
| | methylbenzenesulfonamide) | | | 2H), 7.92 (d, J = 7.78 Hz, 2H), 7.54 (t, |
| | trifluoroacetate | | | J = 7.91 Hz, 2H), 7.46 (q, J = 4.85 Hz, |
| | | | | 2H), 7.37 (d, J = 7.78 Hz, 2H), 6.24 (s, |
| | | | | 1H), 2.45 (d, J = 4.52 Hz, 6H) |
| 6 | 3-({6-[(4-chlorophenyl)amino]-4- | 6.57^{b} | 433.1 (M + H)⁺ | ¹H NMR (400 MHz, DMSO-d₆) δ ppm |
| | pyrimidinyl}amino)-5- | | | 9.51 (br. s., 2 H), 8.36 (s, 1 H), |
| | (dimethylamino)-N- | | | 7.61 (d, J = 8.78 Hz, 2 H), 7.32-7.39 (m, 4 |
| | methylbenzenesulfonamide | | | H), 7.16 (br. s., 1 H), 6.70-6.75 (m, 1 |
| | trifluoroacetate | | | H), 6.17 (s, 1 H), 2.97 (s, 6 H), |
| | | | | 2.43 (d, J = 5.02 Hz, 3 H) |
| 7 | 3-chloro-5-({6-[(4- | 7.22^{b} | 424.0 (M + H)⁺ | ¹H NMR (400 MHz, DMSO-d₆) δ ppm |
| | chlorophenyl)amino]-4- | | | 9.83 (s, 1 H), 9.51 (s, 1 H), 8.42 (s, 1 |
| | pyrimidinyl}amino)-N- | | | H), 8.22-8.29 (m, 1 H), |
| | methylbenzenesulfonamide | | | 7.92-7.99 (m, 1 H), 7.60-7.67 (m, 3 H), |
| | | | | 7.34-7.41 (m, 2 H), 7.30-7.34 (m, 1 H), |
| | | | | 6.20 (s, 1 H), 2.47 (d, J = 5.02 Hz, 3 H) |
| 8 | 3-({6-[(4-chlorophenyl)amino]-4- | 1.12^{d} | 448.2 (M + H)⁺ | ¹H NMR (400 MHz, DMSO-d₆) δ ppm |
| | pyrimidinyl}amino)-N-methyl-4- | | | 9.44 (br. s., 1 H), 8.75 (br. s., 1 H), |
| | (propyloxy)benzenesulfonamide | | | 8.28 (s, 1 H), 8.14 (d, J = 1.98 Hz, 1 H), |
| | trifluoroacetate | | | 7.58 (d, J = 8.82 Hz, 2 H), 7.48 (dd, |
| | | | | J = 8.60, 1.98 Hz, 1 H), 7.33 (d, J = 8.82 Hz, |
| | | | | 2 H), 7.30 (q, J = 5.07 Hz, 1 H), |
| | | | | 7.23 (d, J = 8.82 Hz, 1 H), 6.11 (s, 1 H), |
| | | | | 4.06 (t, J = 6.39 Hz, 2 H), 2.39 (d, |
| | | | | J = 5.07 Hz, 3 H), 1.72 (d, J = 7.06 Hz, 2 |
| | | | | H), 0.90 (t, J = 7.39 Hz, 3 H) |
| 9 | 3-({6-[(4-chlorophenyl)amino]-4- | 1.07^{d} | 434.2 (M + H)⁺ | ¹H NMR (400 MHz, DMSO-d₆) δ ppm |
| | pyrimidinyl}amino)-4-(ethyloxy)- | | | 9.34 (s, 1 H), 8.62 (br. s., 1 H), |
| | N-methylbenzenesulfonamide | | | 8.27 (s, 2 H), 7.54-7.62 (m, 2 H), |
| | trifluoroacetate | | | 7.43 (dd, J = 8.49, 2.09 Hz, 1 H), |
| | | | | 7.30-7.35 (m, 2 H), 7.28 (q, J = 5.07 Hz, 1 H), |
| | | | | 7.21 (d, J = 8.60 Hz, 1 H), 6.19 (s, 1H), |
| | | | | 4.18 (q, J = 6.98 Hz, 2 H), 2.39 (d, |
| | | | | J = 5.07 Hz, 3 H), 1.34 (t, J = 6.95 Hz, 3 |
| | | | | H) |
| 10 | 3-({6-[(4-chlorophenyl)amino]-4- | 1.16^{d} | 462.3 (M + H)⁺ | ¹H NMR (400 MHz, DMSO-d₆) δ ppm |
| | pyrimidinyl}amino)-N-methyl-4- | | | 9.29 (s, 1 H), 8.54 (br. s., 1 H), |
| | [(2- | | | 8.24 (s, 1 H), 8.10 (d, J = 2.43 Hz, 1 H), |
| | methylpropyl)oxy]benzenesulfonamide | | | 7.59 (d, J = 9.04 Hz, 2 H), 7.47 (dd, J = 8.49, |
| | trifluoroacetate | | | 2.32 Hz, 1 H), 7.30 (m, 3 H), 7.22 (d, |
| | | | | J = 8.60 Hz, 1 H), 6.05 (s, 1 H), 3.86 (d, |
| | | | | J = 6.39 Hz, 2 H), 2.39 (d, J = 5.07 Hz, 3 |
| | | | | H), 2.01 (m, 1H), 0.91 (d, J = 6.62 Hz, |
| | | | | 6 H) |
| 11 | 3-({6-[(4-chlorophenyl)amino]-4- | 1.18^{d} | 476.3 (M + H)⁺ | ¹H NMR (400 MHz, DMSO-d₆) δ ppm |
| | pyrimidinyl}amino)-4-[(1,2- | | | 9.49 (br. s., 1 H), 8.71 (br. s., 1 H), |
| | dimethylpropyl)oxy]-N- | | | 8.28 (s, 1 H), 8.04 (s, 1 H), 7.57 (d, |
| | methylbenzenesulfonamide | | | J = 8.82 Hz, 2 H), 7.50 (dd, J = 8.71, |
| | trifluoroacetate | | | 2.09 Hz, 1 H), 7.25-7.35 (m, 4 H), |
| | | | | 6.03 (s, 1 H), 4.42 (m, 1 H), 2.40 (m, |
| | | | | J = 4.85 Hz, 3 H), 1.85 (m, 1 H), |
| | | | | 1.17 (d, J = 6.17 Hz, 3 H), 0.85 (t, J = 6.73 Hz, |
| | | | | 6 H) |
| 12 | 4-chloro-3-({6-[(4- | 6.70^{b} | 424.0 (M + H)⁺ | ¹H NMR (400 MHz, DMSO-d₆) δ ppm |
| | chlorophenyl)amino]-4- | | | 9.52 (br. s., 1 H), 9.19 (br. s., 1 H), |
| | pyrimidinyl}amino)-N- | | | 8.30 (s, 1 H), 8.21 (d, J = 2.01 Hz, 1 H), |
| | methylbenzenesulfonamide | | | 7.76 (d, J = 8.28 Hz, 1 H), |
| | trifluoroacetate | | | 7.58-7.65 (m, 3 H), 7.48-7.55 (m, 1 H), |
| | | | | 7.35-7.42 (m, 2 H), 6.23 (s, 1 H), 2.46 (d, |
| | | | | J = 5.02 Hz, 3 H) |
| 13 | 3-({6-[(4-chlorophenyl)amino]-4- | 1.15^{d} | 488.0 (M + H)⁺ | ¹H NMR (400 MHz, DMSO-d₆) δ ppm |
| | pyrimidinyl}amino)-N-methyl-4- | | | 9.58 (br. s., 1 H), 9.07 (br. s., 1 H), |
| | [(2,2,2- | | | 8.29 (s, 1 H), 8.07 (d, J = 2.21 Hz, 1 H), |
| | trifluoroethyl)oxy]benzenesulfonamide | | | 7.52-7.59 (m, 3 H), 7.38-7.44 (m, 2 |
| | trifluoroacetate | | | H), 7.31-7.38 (m, 2 H), 6.11 (s, 1 H), |
| | | | | 4.89 (q, J = 8.82 Hz, 2 H), 2.41 (d, |
| | | | | J = 4.85 Hz, 3 H) |
| 14 | 3-({6-[(4-chlorophenyl)amino]-4- | 1.23^{d} | 488.2 (M + H)⁺ | ¹H NMR (400 MHz, DMSO-d₆) δ ppm |
| | pyrimidinyl}amino)-4- | | | 9.46 (br. s., 1 H), 8.65-8.72 (br. s, 1 |
| | (cyclohexyloxy)-N- | | | H), 8.28 (s, 1 H), 8.13 (d, J = 2.21 Hz, 1 |
| | methylbenzenesulfonamide | | | H), 7.54-7.61 (m, 2 H), 7.46 (dd, |
| | trifluoroacetate | | | J = 8.71, 2.32 Hz, 1 H), 7.26-7.35 (m, |
| | | | | 4 H), 6.11 (s, 1 H), 4.46-4.53 (m, 1 |
| | | | | H), 2.40 (d, J = 5.07 Hz, 3 H), 1.86 (m, |
| | | | | 2 H), 1.63 (m, 2 H), 1.47 (m, 3 H), |
| | | | | 1.31-1.38 (m, 2 H), 1.24 (m, 1 H) |
| 15 | 3-({6-[(4-chlorophenyl)amino]-4- | 1.19^{d} | 476.3 (M + H)⁺ | ¹H NMR (400 MHz, DMSO-d₆) δ ppm |
| | pyrimidinyl}amino)-4-[(1- | | | 9.47 (br. s., 1 H), 8.69 (br. s., 1 H), |
| | ethylpropyl)oxy]-N- | | | 8.27 (s, 1 H), 8.10 (br. s., 1 H), |
| | methylbenzenesulfonamide | | | 7.55 (d, J = 9.04 Hz, 2 H), 7.46 (m., 1 H), |
| | trifluoroacetate | | | 7.22-7.33 (m, 4 H), 6.08 (s, 1 H), |
| | | | | 4.36 (m, 1 H), 2.39 (d, J = 4.85 Hz, 3 |
| | | | | H), 1.56-1.63 (m, 4 H), 0.82 (t, |
| | | | | J = 7.39 Hz, 6 H) |
| 16 | 3-({6-[(4-chlorophenyl)amino]-4- | 1.13^{d} | 502.0 (M + H)⁺ | ¹H NMR (400 MHz, DMSO-d₆) δ ppm |
| | pyrimidinyl}amino)-N-methyl-4- | | | 9.37 (br. s., 1 H), 8.48 (br. s., 1 H), |
| | [(3,3,3- | | | 8.30-8.36 (m, 1 H), 8.27 (s, 1 H), |
| | trifluoropropyl)oxy]benzenesulfonamide | | | 7.58 (d, J = 8.82 Hz, 2 H), |
| | trifluoroacetate | | | 7.40-7.46 (m, 1 H), 7.25-7.33 (m, 4 H), 6.14 (s, |
| | | | | 1 H), 4.32 (t, J = 5.95 Hz, 2 H), |
| | | | | 2.82 (m, 2 H), 2.38 (d, J = 4.85 Hz, 3 H) |
| 17 | 3-({6-[(4-chlorophenyl)amino]-4- | 1.16^{d} | 474.2 (M + H)⁺ | ¹H NMR (400 MHz, DMSO-d₆) δ ppm |
| | pyrimidinyl}amino)-4- | | | 9.55 (br. s., 1 H), 8.81 (br. s., 1 H), |
| | (cyclopentyloxy)-N- | | | 8.30 (s, 1 H), 8.04-8.11 (m, 1 H), |
| | methylbenzenesulfonamide | | | 7.58 (d, J = 8.78 Hz, 2 H), 7.51 (dd, |
| | trifluoroacetate | | | J = 8.66, 1.88 Hz, 1 H), 7.30-7.37 (m, |
| | | | | 3 H), 7.23 (d, J = 8.78 Hz, 1 H), 6.07 (s, |
| | | | | 1 H), 4.91-4.98 (m, 1 H), 2.41 (d, |
| | | | | J = 4.77 Hz, 3 H), 1.91 (m, 2 H), |
| | | | | 1.75 (m, 2 H), 1.62 (m, 2 H), 1.54 (m, 2 H) |
| 18 | 5-(6-(4- | 1.04^{c} | 438.0 (M + H)⁺ | ¹H NMR (400 MHz, DMSO-d₆) δ ppm |
| | chlorophenylamino)pyrimidin-4- | | | 2.47 (d, 3H, obscured by solvent) |
| | ylamino)-2-fluoro-4-methoxy-N- | | | 3.89 (s, 3 H) 6.08 (s, 1 H) 7.26 (d, J = 11.91 Hz, |
| | methylbenzenesulfonamide | | | 1 H) 7.35 (d, J = 8.82 Hz, 2 H) |
| | trifluoroacetate | | | 7.53 (d, J = 8.82 Hz, 2 H) 7.59 (q, J = 4.85 Hz, |
| | | | | 1 H) 8.05 (d, J = 7.94 Hz, 1 H) |
| | | | | 8.28 (s, 1 H) 9.07 (br. s., 1 H) 9.61 (br. s., 1 |
| | | | | H) |
| 19 | 3-({6-[(4-chlorophenyl)amino]-4- | 1.75^{a} | 501.1 (M + H)⁺ | ¹H NMR (400 MHz, DMSO-d₆) δ ppm |
| | pyrimidinyl}amino)-N-methyl-4- | | | 2.42 (d, J = 5.02 Hz, 3 H) 2.99 (s, 3 H) |
| | [methyl(2,2,2- | | | 4.00 (q, J = 9.79, 2 H) 5.90 (s, 1 H) |
| | trifluoroethyl)amino]benzenesulfonamide | | | 7.33-7.42 (m, 4 H) 7.55 (dd, J = 8.53, |
| | trifluoroacetate | | | 2.26 Hz, 1 H) 7.59 (d, J = 8.78 Hz, 2 H) |
| | | | | 7.84 (d, J = 2.01 Hz, 1 H) 8.31 (s, 1 H) |
| | | | | 8.98 (br. s., 1 H) 9.53 (br. s., 1 H) |
| 20 | 1-{6-[(4-chlorophenyl)amino]-4- | 2.46^{a} | 444.1 (M + H)⁺ | ¹H NMR (400 MHz, DMSO-d₆) δ ppm |
| | pyrimidinyl}-N,3,3-trimethyl-2,3- | | | 1.38 (s, 6 H) 2.43 (d, J = 4.27 Hz, 3 H) |
| | dihydro-1H-indole-6- | | | 6.07 (br. s., 1 H) 7.33-7.75 (m, 8 H) |
| | sulfonamide trifluoroacetate | | | 8.46 (s, 1 H) 8.78 (br. s., 1 H) |
| | | | | 9.56 (br. s., 1 H) |
| 21 | 3-({6-[(4-chlorophenyl)amino]-4- | 2.31^{a} | 502.0 (M + H)⁺ | ¹H NMR (400 MHz, METHANOL-d₄) δ |
| | pyrimidinyl}amino)-N-methyl-4- | | | ppm 1.54 (d, J = 6.27 Hz, 3 H) 2.57 (s, |
| | [(2,2,2-trifluoro-1- | | | 3 H) 5.20 (dt, J = 12.49, 6.18 Hz, 1 H) |
| | methylethyl)oxy]benzenesulfonamide | | | 6.15 (s, 1 H) 7.35 (d, J = 9.03 Hz, 2 H) |
| | trifluoroacetate | | | 7.39 (d, J = 8.78 Hz, 1 H) 7.47 (d, |
| | | | | J = 9.03 Hz, 2 H) 7.65 (dd, J = 8.78, |
| | | | | 2.26 Hz, 1 H) 8.23 (d, J = 2.26 Hz, 1 H) |
| | | | | 8.26 (d, J = 0.75 Hz, 1 H) |
| 22 | 5-(6-(4- | 1.13^{c} | 506.1 (M + H)⁺ | ¹H NMR (400 MHz, DMSO-d₆) δ ppm |
| | chlorophenylamino)pyrimidin-4- | | | 2.47 (d, 3H, obscured by solvent) |
| | ylamino)-2-fluoro-N-methyl-4- | | | 4.91 (q, J = 8.82 Hz, 2 H) 6.02 (s, 1 H) |
| | (2,2,2- | | | 7.33 (d, 2 H) 7.44 (d, J = 11.69 Hz, 1 H) |
| | trifluoroethoxy)benzenesulfonamide | | | 7.57 (d, J = 9.04 Hz, 2 H) 7.69 (q, |
| | trifluoroacetate | | | J = 4.85 Hz, 1 H) 7.93 (d, J = 7.72 Hz, 1 |
| | | | | H) 8.21-8.26 (m, 1 H) 8.92 (br. s., 1 |
| | | | | H) 9.48 (br. s., 1 H) |
| 23 | 4-amino-3-({6-[(4- | 1.91^{a} | 405.0 (M + H)⁺ | ¹H NMR (400 MHz, DMSO-d₆) δ ppm |
| | chlorophenyl)amino]-4- | | | 2.37 (d, J = 4.02 Hz, 3 H) 5.70 (br. s., 1 |
| | pyrimidinyl}amino)-N- | | | H) 6.88 (d, J = 8.53 Hz, 1 H) |
| | methylbenzenesulfonamide | | | 7.07-7.15 (m, 1 H) 7.37-7.58 (m, 6 H) |
| | trifluoroacetate | | | 8.41 (s, 1 H) 9.38 (br. s., 1 H) |
| | | | | 10.02 (br. s., 1 H) |
| 24 | 5-[6-(4-chloro-phenylamino)- | 1.06^{c} | 451.1 (M + H)⁺ | ¹H NMR (400 MHz, DMSO-d₆) δ ppm |
| | pyrimidin-4-ylamino]-4- | | | 2.45 (d, J = 4.85 Hz, 3 H) 2.78 (s, 6 H) |
| | dimethylamino-2-fluoro-N- | | | 5.79 (s, 1 H) 6.91 (d, J = 13.23 Hz, 1 H) |
| | methyl-benzenesulfonamide | | | 7.29 (d, J = 8.82 Hz, 2 H) 7.47 (q, |
| | | | | J = 4.92 Hz, 1 H) 7.54-7.60 (m, 3 H) |
| | | | | 8.20 (s, 1 H) 8.69 (br. s., 1 H) |
| | | | | 9.31 (br. s., 1 H) |
| 25 | 3-({6-[(4-chlorophenyl)amino]-4- | 2.30^{a} | 509.1 (M + H)⁺ | ¹H NMR (400 MHz, DMSO-d₆) δ ppm |
| | pyrimidinyl}amino)-4-(3,3- | | | 1.67-1.75 (m, 2 H) 1.94-2.06 (m, 2 |
| | difluoro-1-piperidinyl)-N- | | | H) 2.43 (d, J = 5.02 Hz, 3 H) 3.03 (d, |
| | methylbenzenesulfonamide | | | J = 5.02 Hz, 2 H) 3.27 (t, J = 11.54 Hz, 2 |
| | trifluoroacetate | | | H) 5.99 (s, 1 H) 7.34 (d, J = 8.53 Hz, 1 |
| | | | | H) 7.38 (d, J = 8.78 Hz, 2 H) 7.43 (q, |
| | | | | J = 4.94 Hz, 1 H) 7.56 (d, J = 8.53 Hz, 1 |
| | | | | H) 7.59 (d, J = 8.78 Hz, 2 H) 7.94 (br. |
| | | | | s., 1 H) 8.34 (s, 1 H) 8.93 (br. s., 1 H) |
| | | | | 9.68 (br. s., 1 H) |
| 26 | 3-({6-[(4-chlorophenyl)amino]-4- | 1.88^{a} | 556.1 (M + H)⁺ | ¹H NMR (400 MHz, DMSO-d₆) δ ppm |
| | pyrimidinyl}amino)-N-methyl-4- | | | 2.46 (d, J = 5.02 Hz, 3 H) 6.12 (s, 1 H) |
| | {[2,2,2-trifluoro-1- | | | 6.61-6.73 (m, 1 H) 7.36 (d, J = 8.78 Hz, |
| | (trifluoromethyl)ethyl]oxy}benzenesulfonamide | | | 2 H) 7.51 (d, J = 5.02 Hz, 1 H) |
| | trifluoroacetate | | | 7.58-7.65 (m, 4 H) 8.12 (s, 1 H) 8.28 (s, 1 |
| | | | | H) 9.04 (br. s., 1 H) 9.51 (br. s., 1 H) |
| 27 | 4-(dimethylamino)-3-({6-[(3- | 5.73^{b} | 417.1 (M + H)⁺ | ¹H NMR (500 MHz, DMSO-d₆) δ ppm |
| | fluorophenyl)amino]-4- | | | 9.67 (br. s., 1 H), 9.09 (br. s., 1 H), |
| | pyrimidinyl}amino)-N- | | | 8.33 (s, 1 H), 7.83 (s, 1 H), 7.57 (d, |
| | methylbenzenesulfonamide | | | J = 11.72 Hz, 1 H), 7.50 (dd, J = 8.55, |
| | trifluoroacetate | | | 1.95 Hz, 1 H), 7.33 (q, J = 7.89 Hz, 1 |
| | | | | H), 7.23-7.29 (m, 2 H), 7.19 (d, |
| | | | | J = 8.79 Hz, 1 H), 6.80-6.86 (m, 1 H), |
| | | | | 6.05 (s, 1 H), 2.77 (s, 6 H), 2.41 (d, |
| | | | | J = 4.64 Hz, 3 H) |
| 28 | 3-({6-[(3-fluorophenyl)amino]-4- | 5.57^{b} | 459.2 (M + H)⁺ | ¹H NMR (500 MHz, DMSO-d₆) δ ppm |
| | pyrimidinyl}amino)-N-methyl-4- | | | 9.62 (br. s., 1 H), 8.92 (br. s., 1 H), |
| | (4- | | | 8.34 (s, 1 H), 7.93 (s, 1 H), 7.65 (m, 1 |
| | morpholinyl)benzenesulfonamide | | | H), 7.53 (dd, J = 8.30, 1.71 Hz, 1 H), |
| | trifluoroacetate | | | 7.31-7.36 (m, 2 H), 7.26 (d, J = 8.55 Hz, |
| | | | | 2 H), 6.78-6.84 (m, 1 H), 6.09 (s, |
| | | | | 1 H), 3.64 (m, 4 H), 2.94-3.00 (m, 4 |
| | | | | H), 2.43 (d, J = 4.88 Hz, 3 H) |
| 29 | 1-{6-[(3-fluorophenyl)amino]-4- | 5.95^{b} | 400.1 (M + H)⁺ | ¹H NMR (500 MHz, DMSO-d₆) δ ppm |
| | pyrimidinyl}-N-methyl-2,3- | | | 9.61 (s, 1 H), 8.81 (s, 1 H), 8.48 (s, 1 |
| | dihydro-1H-indole-6- | | | H), 7.78 (d, J = 12.21 Hz, 1 H), |
| | sulfonamide trifluoroacetate | | | 7.36-7.42 (m, 2 H), 7.29-7.35 (m, 3 H), |
| | | | | 6.75-6.81 (m, 1 H), 6.08 (s, 1 H), |
| | | | | 4.05 (t, J = 8.67 Hz, 2 H), 3.28 (m, 2H), |
| | | | | 2.42 (d, J = 5.13 Hz, 3 H) |
| 30 | 3-({6-[(3-fluorophenyl)amino]-4- | 5.52^{b} | 404.1 (M + H)⁺ | ¹H NMR (500 MHz, DMSO-d₆) δ ppm |
| | pyrimidinyl}amino)-N-methyl-4- | | | 9.44 (br. s., 1 H), 8.80 (br. s., 1 H), |
| | (methyloxy)benzenesulfonamide | | | 8.37 (s, 1 H), 8.32 (s, 1 H), 7.61 (d, |
| | trifluoroacetate | | | J = 11.96 Hz, 1 H), 7.48 (dd, J = 8.67, |
| | | | | 2.08 Hz, 1 H), 7.23-7.31 (m, 4 H), |
| | | | | 6.78-6.81 (m, 1 H), 6.28 (s, 1 H), |
| | | | | 3.92 (s, 3 H), 2.42 (d, J = 4.88 Hz, 3 H) |
| 31 | N-methyl-3-[(6-{[4-(1- | 2.27^{a} | 444.1 (M + H)⁺ | ¹H NMR (400 MHz, DMSO-d₆) δ ppm |
| | methylethyl)phenyl]amino}-4- | | | 9.07 (br. s., 1 H), 8.72 (br. s., 1 H), |
| | pyrimidinyl)amino]-4- | | | 8.15 (s, 1 H), 7.67-7.74 (m, 1 H), |
| | (methylthio)benzenesulfonamide | | | 7.59-7.64 (m, 1 H), 7.44-7.51 (m, 2 |
| | hydrochloride | | | H), 7.40 (d, J = 8.28 Hz, 2 H), 7.16 (d, |
| | | | | J = 8.28 Hz, 2 H), 5.86 (s, 1 H), |
| | | | | 2.83 (m, 1 H), 2.49 (s, 3 H), 2.42 (d, J = 5.02 Hz, |
| | | | | 3 H), 1.18 (d, J = 6.78 Hz, 6 H) |
| 32 | 3-[(6-{[3-chloro-4- | 2.40^{a} | 518.0 (M + H)⁺ | ¹H NMR (400 MHz, DMSO-d₆) δ ppm |
| | (methyloxy)phenyl]amino}-4- | | | 2.49 (d, J = 5.02 Hz, 3 H) 3.88 (s, 3 H) |
| | pyrimidinyl)amino]-N-methyl-4- | | | 4.96 (q, J = 8.78 Hz, 2 H) |
| | [(2,2,2- | | | 6.13-6.16 (m, 1 H) 7.14-7.19 (m, 1 H) |
| | trifluoroethyl)oxy]benzenesulfonamide | | | 7.41-7.48 (m, 3 H) 7.53-7.58 (m, 1 H) |
| | hydrochloride | | | 7.79-7.82 (m, 1 H) 8.25-8.28 (m, 1 |
| | | | | H) 8.28-8.30 (m, 1 H) 8.69-8.72 (m, |
| | | | | 1 H) 9.16-9.18 (m, 1 H) |
| 33 | 3-[(6-{[3-chloro-4- | 2.21^{a} | 450.0 (M + H)⁺ | ¹H NMR (400 MHz, DMSO-d₆) δ ppm |
| | (methyloxy)phenyl]amino}-4- | | | 2.47 (d, J = 5.02 Hz, 3 H) 3.91 (s, 3 H) |
| | pyrimidinyl)amino]-N-methyl-4- | | | 3.98 (s, 3 H) 6.14 (s, 1 H) 7.22 (d, |
| | (methyloxy)benzenesulfonamide | | | J = 9.03 Hz, 1 H) 7.36 (d, J = 8.78 Hz, 1 |
| | trifluoroacetate | | | H) 7.41 (dd, J = 8.91, 2.64 Hz, 2 H) |
| | | | | 7.63 (dd, J = 8.66, 2.13 Hz, 1 H) |
| | | | | 7.70 (d, J = 2.51 Hz, 1 H) 8.20 (br. s., 1 H) |
| | | | | 8.40 (s, 1 H) 9.39 (br. s., 1 H) |
| | | | | 9.72 (br. s., 1 H) |
| 34 | N-methyl-4-(methyloxy)-3-({6- | 2.02^{a} | 460.1 (M + H)⁺ | ¹H NMR (400 MHz, DMSO-d₆) δ ppm |
| | [(4-{[2- | | | 2.40 (d, J = 4.77 Hz, 3 H) 3.32 (s, 3 H) |
| | (methyloxy)ethyl]oxy}phenyl)amino]- | | | 3.64-3.70 (m, 2 H) 3.91 (s, 3 H) |
| | 4- | | | 4.10 (dd, J = 5.27, 3.76 Hz, 2 H) 6.07 (br. s., |
| | pyrimidinyl}amino)benzenesulfonamide | | | 1 H) 7.00 (d, J = 8.78 Hz, 2 H) |
| | hydrochloride | | | 7.33 (dd, J = 8.78, 4.52 Hz, 3 H) 7.44 (q, |
| | | | | J = 4.60 Hz, 1 H) 7.65 (dd, J = 8.78, |
| | | | | 2.26 Hz, 1 H) 7.93 (br. s., 1 H) |
| | | | | 8.39 (s, 1 H) 9.83 (br. s., 1 H) 10.16 (br. s., |
| | | | | 1 H) |
| 35 | N-methyl-3-({6-[(4-{[2- | 2.22^{a} | 528.0 (M + H)⁺ | ¹H NMR (400 MHz, DMSO-d₆) δ ppm |
| | (methyloxy)ethyl]oxy}phenyl)amino]- | | | 2.42 (d, J = 5.02 Hz, 3 H) |
| | 4-pyrimidinyl}amino)-4- | | | 3.64-3.68 (m, 2 H) 4.08 (dd, J = 5.52, 3.76 Hz, 2 |
| | [(2,2,2- | | | H) 4.91 (d, J = 8.78 Hz, 2 H) |
| | trifluoroethyl)oxy]benzenesulfonamide | | | 5.98-6.02 (m, 1 H) 6.97 (d, J = 9.03 Hz, 1 H) |
| | trifluoroacetate | | | 7.32 (s, 1 H) 7.41-7.46 (m, 1 H) |
| | | | | 7.58-7.63 (m, 1 H) 7.99-8.02 (m, 1 H) |
| | | | | 8.28 (s, 1 H) 9.30 (br. s., 1 H) |
| | | | | 9.55 (br. S., 1 H) |
| 36 | N-methyl-4-(methyloxy)-3-[(6- | 2.30^{a} | 468.1 (M + H)⁺ | ¹H NMR (500 MHz, DMSO-d₆) δ ppm |
| | {[4-(2,2,2- | | | 2.41 (d, J = 4.88 Hz, 3 H) 3.58 (q, |
| | trifluoroethyl)phenyl]amino}-4- | | | J = 11.72 Hz, 2 H) 3.92 (s, 3 H) 6.26 (s, |
| | pyrimidinyl)amino]benzenesulfonamide | | | 1 H) 7.23-7.33 (m, 4 H) |
| | trifluoroacetate | | | 7.47-7.53 (m, 3 H) 8.30 (br. s., 2 H) 8.98 (br. s., |
| | | | | 1 H) 9.46 (br. s., 1 H) |
| 37 | N-methyl-4-[(2,2,2- | 2.42^{a} | 536.1 (M + H)⁺ | ¹H NMR (400 MHz, DMSO-d₆) δ ppm |
| | trifluoroethyl)oxy]-3-[(6-{[4- | | | 2.43 (d, J = 4.52 Hz, 3 H) 3.62 (q, |
| | (2,2,2- | | | J = 11.54 Hz, 2 H) 4.92 (q, J = 8.70 Hz, |
| | trifluoroethyl)phenyl]amino}-4- | | | 2 H) 6.16 (s, 1 H) 7.35 (d, J = 8.28 Hz, |
| | pyrimidinyl)amino]benzenesulfonamide | | | 2 H) 7.43-7.52 (m, 4 H) 7.64 (dd, |
| | trifluoroacetate | | | J = 8.78, 2.26 Hz, 1 H) 7.99 (d, J = 2.01 Hz, |
| | | | | 1 H) 8.37 (s, 1 H) 9.57 (br. s., 1 H) |
| | | | | 9.95 (br. s., 1 H) |
| 38 | N-methyl-3-[(6-{[4-(2,2,2- | 2.36^{a} | 552.1 (M + H)⁺ | ¹H NMR (400 MHz, DMSO-d₆) δ ppm |
| | trifluoroethyl)phenyl]amino}-4- | | | 2.44 (d, J = 5.02 Hz, 3 H) 3.57 (q, |
| | pyrimidinyl)amino]-4-[(2,2,2- | | | J = 11.71 Hz, 2 H) 4.10 (q, J = 10.37 Hz, |
| | trifluoroethyl)thio]benzenesulfonamide | | | 2 H) 5.99 (s, 1 H) 7.27 (d, J = 8.28 Hz, |
| | trifluoroacetate | | | 2 H) 7.50-7.60 (m, 4 H) 7.78 (d, |
| | | | | J = 2.01 Hz, 1 H) 7.82 (d, J = 8.53 Hz, 1 |
| | | | | H) 8.21 (s, 1 H) 9.04 (s, 1 H) 9.34 (s, |
| | | | | 1 H) |
| 39 | 4-[(6-{[5-[(methylamino)sulfonyl]- | 1.92^{a} | 503.1 (M + H)⁺ | ¹H NMR (400 MHz, DMSO-d₆) δ ppm |
| | 2-(methylthio)phenyl]amino}-4- | | | 2.44 (d, J = 5.02 Hz, 3 H) 2.47 (s, 3H, |
| | pyrimidinyl)amino]-N-[2- | | | obscured by solvent) 3.38-3.49 (m, 4 |
| | (methyloxy)ethyl]benzamide | | | H) 3.97 (s, 3 H) 5.91 (s, 1 H) 7.47 (q, |
| | trifluoroacetate | | | J = 4.85 Hz, 1 H) 7.54 (d, J = 8.28 Hz, 1 |
| | | | | H) 7.61-7.69 (m, 4 H) 7.80 (d, J = 8.78 Hz, |
| | | | | 2 H) 8.30 (s, 1 H) 8.34-8.38 (m, |
| | | | | 1 H) 9.16 (br. s., 1 H) 9.67 (br. s., 1 H) |
| 40 | N-methyl-4-(methyloxy)-3-[(6- | 2.04^{a} | 452.1 (M + H)⁺ | ¹H NMR (400 MHz, DMSO-d₆) δ ppm |
| | {[4-(1H-pyrazol-1- | | | 2.42 (d, J = 4.77 Hz, 3 H) 3.93 (s, 3 H) |
| | yl)phenyl]amino}-4- | | | 6.22 (s, 1 H) 6.51-6.57 (m, 1 H) |
| | pyrimidinyl)amino]benzenesulfonamide | | | 7.30 (d, J = 8.78 Hz, 1 H) 7.34 (q, J = 4.77 Hz, |
| | trifluoroacetate | | | 1 H) 7.56 (dd, J = 8.53, 2.26 Hz, 1 |
| | | | | H) 7.62 (d, J = 9.03 Hz, 2 H) 7.73 (d, |
| | | | | J = 1.51 Hz, 1 H) 7.82 (d, J = 9.03 Hz, 2 |
| | | | | H) 8.22 (s, 1 H) 8.37 (s, 1 H) 8.44 (d, |
| | | | | J = 2.51 Hz, 1 H) 9.24 (br. s., 1 H) |
| | | | | 9.75 (br. s., 1 H) |
| 41 | N-methyl-3-[(6-{[4-(1H-pyrazol- | 2.24^{a} | 520.0 (M + H)⁺ | ¹H NMR (400 MHz, DMSO-d₆) δ ppm |
| | 1-yl)phenyl]amino}-4- | | | 2.44 (d, J = 5.02 Hz, 3 H) 4.92 (q, |
| | pyrimidinyl)amino]-4-[(2,2,2- | | | J = 8.78 Hz, 2 H) 6.18 (s, 1 H) |
| | trifluoroethyl)oxy]benzenesulfonamide | | | 6.51-6.55 (m, 1 H) 7.37-7.43 (m, 2 H) |
| | trifluoroacetate | | | 7.50-7.54 (m, 1 H) 7.70 (d, J = 8.78 Hz, |
| | | | | 3 H) 7.75 (s, 2 H) 8.19-8.21 (m, |
| | | | | 1 H) 8.28 (s, 1 H) 8.39-8.42 (m, 1 H) |
| | | | | 8.74 (br. s., 1 H) 9.36 (br. s., 1 H) |
| 42 | N-methyl-4-[(2,2,2- | 1.77^{a} | 552.2 (M + H)⁺ | ¹H NMR (500 MHz, DMSO-d₆) δ ppm |
| | trifluoroethyl)oxy]-3-{[6-({4- | | | 2.42 (d, J = 4.88 Hz, 3 H) 4.72 (q, |
| | [(2,2,2- | | | J = 9.03 Hz, 2 H) 4.90 (q, J = 8.79 Hz, 2 |
| | trifluoroethyl)oxy]phenyl}amino)- | | | H) 6.05 (s, 1 H) 7.05 (d, J = 8.79 Hz, 2 |
| | 4- | | | H) 7.34-7.58 (m, 5 H) 8.10 (br. s., 1 |
| | pyrimidinyl]amino}benzenesulfonamide | | | H) 8.25 (s, 1 H) 8.99 (none, 1 H) |
| | trifluoroacetate | | | 9.29-9.40 (m, 1 H) |
| 43 | N-methyl-4-[(2,2,2- | 1.88^{a} | 522.1 (M + H)⁺ | ¹H NMR (400 MHz, DMSO-d₆) δ ppm |
| | trifluoroethyl)oxy]-3-[(6-{[4- | | | 2.44 (d, J = 5.02 Hz, 3 H) 4.92 (q, |
| | (trifluoromethyl)phenyl]amino}-4- | | | J = 8.78 Hz, 2 H) 6.21 (s, 1 H) |
| | pyrimidinyl)amino]benzenesulfonamide | | | 7.37-7.45 (m, 2 H) 7.55 (dd, J = 8.53, 2.26 Hz, |
| | trifluoroacetate | | | 1 H) 7.64 (d, J = 8.53 Hz, 2 H) |
| | | | | 7.83 (d, J = 8.53 Hz, 2 H) 8.15 (d, J = 2.01 Hz, |
| | | | | 1 H) 8.33 (s, 1 H) 8.87 (br. s., 1 H) |
| | | | | 9.64 (br. s., 1 H) |
| 44 | 3-({6-[(3,4- | 2.21^{a} | 409.9 (M + H)⁺ | ¹H NMR (400 MHz, DMSO-d₆) δ ppm |
| | difluorophenyl)amino]-4- | | | 2.45 (d, J = 4.52 Hz, 3 H) 6.26 (s, 1 H) |
| | pyrimidinyl}amino)-4-fluoro-N- | | | 7.22-7.30 (m, 1 H) 7.32-7.46 (m, 1 |
| | methylbenzenesulfonamide | | | H) 7.52 (d, J = 7.78 Hz, 3 H) |
| | trifluoroacetate | | | 7.76-7.86 (m, 1 H) 8.37 (s, 1 H) 8.42 (br. s., |
| | | | | 1 H) 9.52 (br. s., 1 H) 9.70 (br. s., 1 H) |
| 45 | 3-({6-[(3,4- | 2.35^{a} | 503.9 (M + H)⁺ | ¹H NMR (400 MHz, DMSO-d₆) δ ppm |
| | difluorophenyl)amino]-4- | | | 1.45 (d, J = 6.27 Hz, 3 H) 2.44 (d, |
| | pyrimidinyl}amino)-N-methyl-4- | | | J = 4.52 Hz, 3 H) 5.32-5.44 (m, 1 H) |
| | [(2,2,2-trifluoro-1- | | | 6.11 (s, 1 H) 7.23-7.28 (m, 1 H) |
| | methylethyl)oxy]benzenesulfonamide | | | 7.30-7.39 (m, 1 H) 7.41 (q, J = 4.85 Hz, 1 |
| | trifluoroacetate | | | H) 7.49 (m, J = 7.28 Hz, 2 H) |
| | | | | 7.83-7.92 (m, 1 H) 8.19 (d, J = 2.01 Hz, 1 H) |
| | | | | 8.28 (s, 1 H) 8.64 (s, 1 H) 9.39 (s, 1 |
| | | | | H) |
| 46 | 1-{6-[(3,4-difluorophenyl)amino]- | 2.52^{a} | 445.9 (M + H)⁺ | ¹H NMR (400 MHz, DMSO-d₆) δ ppm |
| | 4-pyrimidinyl}-N,3,3-trimethyl- | | | 1.39 (s, 6 H) 2.43 (d, J = 4.52 Hz, 3 H) |
| | 2,3-dihydro-1H-indole-6- | | | 3.80 (s, 2 H) 6.05 (s, 1 H) |
| | sulfonamide trifluoroacetate | | | 7.28-7.50 (m, 5 H) 7.90-7.99 (m, 1 H) 8.49 (s, |
| | | | | 1 H) 8.78 (d, J = 1.51 Hz, 1 H) 9.68 (s, |
| | | | | 1 H) |
| 47 | 3-[6-(6-bromo-4-methyl-pyridin- | 1.16^{c} | 548.8 (M + H)⁺ | ¹H NMR (400 MHz, DMSO-d₆) δ ppm |
| | 2-ylamino)-pyrimidin-4-ylamino]- | | | 2.24 (s, 3 H) 2.40 (d, J = 5.07 Hz, 3 H) |
| | N-methyl-4-(2,2,2-trifluoro- | | | 4.87 (q, J = 8.23 Hz, 2 H) 6.83 (s, 1 H) |
| | ethoxy)-benzenesulfonamide | | | 7.00 (s, 1 H) 7.40 (m, J = 8.60 Hz, 2 H) |
| | trifluoroacetate | | | 7.53 (m, J = 13.67 Hz, 2 H) 7.94 (d, |
| | | | | J = 1.98 Hz, 1 H) 8.28 (s, 1 H) 9.04 (br. |
| | | | | s., 1 H) 10.08 (br. s., 1 H) |
| 48 | 3-({6-[(3,5-dichloro-2- | 1.75^{a} | 523.1 (M + H)⁺ | ¹H NMR (400 MHz, DMSO-d₆) δ ppm |
| | pyridinyl)amino]-4- | | | 2.44 (d, J = 5.02 Hz, 3 H) 4.92 (q, |
| | pyrimidinyl}amino)-N-methyl-4- | | | J = 8.78 Hz, 2 H) 7.22 (s, 1 H) |
| | [(2,2,2- | | | 7.40-7.46 (m, 2 H) 7.60 (dd, J = 8.78, 2.26 Hz, |
| | trifluoroethyl)oxy]benzenesulfonamide | | | 1 H) 8.08 (d, J = 2.26 Hz, 1 H) |
| | trifluoroacetate | | | 8.28 (d, J = 2.26 Hz, 1 H) 8.35-8.39 (m, 2 |
| | | | | H) 9.12 (br. s., 1 H) 9.39 (br. s., 1 H) |
| 49 | 3-{[6-(3-biphenylylamino)-4- | 2.26^{a} | 432.1 (M + H)⁺ | ¹H NMR (400 MHz, DMSO-d6) δ ppm |
| | pyrimidinyl]amino}-N- | | | 9.84 (br. s., 1H), 9.67 (br. s., 1H), |
| | methylbenzenesulfonamide | | | 8.41 (s, 1H), 8.04 (s, 1H), 7.88 (d, J = 8.06 Hz, |
| | trifluoroacetate | | | 1H), 7.79 (s, 1H), 7.66 (d, J = 7.55 Hz, |
| | | | | 2H), 7.42-7.58 (m, 6H), |
| | | | | 7.33-7.42 (m, 3H), 6.24 (s, 1H), 2.44 (d, J = 4.78 Hz, |
| | | | | 3H) |
| 50 | N-methyl-3-({6-[(4- | 2.03^{a} | 370.1 (M + H)⁺ | ¹H NMR (400 MHz, DMSO-d6) δ ppm |
| | methylphenyl)amino]-4- | | | 9.49 (s, 1H), 9.13 (s, 1H), 8.30 (s, |
| | pyrimidinyl}amino)benzenesulfonamide | | | 1H), 8.07-8.14 (m, 1H), |
| | hydrochloride | | | 7.85-7.92 (m, 1H), 7.50 (t, J = 8.03 Hz, 1H), |
| | | | | 7.37-7.46 (m, 3H), 7.31 (d, J = 7.78 Hz, |
| | | | | 1H), 7.13 (d, J = 8.28 Hz, 2H), |
| | | | | 6.15 (s, 1H), 2.44 (d, J = 5.02 Hz, 3H), |
| | | | | 2.27 (s, 3H) |
| 51 | 3-{[6-({3- | 1.66^{a} | 399.1 (M + H)⁺ | ¹H NMR (400 MHz, DMSO-d6) δ ppm |
| | [(methylamino)sulfonyl]phenyl}amino)- | | | 9.58 (s, 1H), 9.39 (s, 1H), 8.36 (s, |
| | 4- | | | 1H), 8.11 (t, J = 1.88 Hz, 1H), |
| | pyrimidinyl]amino}benzamide | | | 7.98-8.01 (m, 1H), 7.89-7.96 (m, 2H), |
| | | | | 7.76-7.81 (m, 1H), 7.46-7.54 (m, |
| | | | | 2H), 7.43 (q, J = 5.02 Hz, 1H), |
| | | | | 7.31-7.41 (m, 3H), 6.21 (s, 1H), 2.45 (d, J = 5.02 Hz, |
| | | | | 3H) |
| 52 | 3-({6-[(3-acetylphenyl)amino]-4- | 1.90^{a} | 398.1 (M + H)⁺ | ¹H NMR (400 MHz, DMSO-d6) δ ppm |
| | pyrimidinyl}amino)-N- | | | 9.84 (br. s., 1H), 9.76 (br. s., 1H), |
| | methylbenzenesulfonamide | | | 8.42 (s, 1H), 8.06 (s, 1H), 8.10 (s, 1H), |
| | trifluoroacetate | | | 7.84-7.94 (m, 2H), 7.65 (d, J = 7.78 Hz, |
| | | | | 1H), 7.44-7.59 (m, 3H), 7.39 (d, |
| | | | | J = 7.53 Hz, 1H), 6.24 (s, 1H), 2.59 (s, |
| | | | | 3H), 2.45 (d, J = 3.26 Hz, 3H) |
| 53 | N-methyl-3-[(6-{[3- | 2.13^{a} | 386.1 (M + H)⁺ | ¹H NMR (400 MHz, DMSO-d6) δ ppm |
| | (methyloxy)phenyl]amino}-4- | | | 9.77 (s, 1H), 9.49 (br. s., 1H), 8.38 (s, |
| | pyrimidinyl)amino]benzenesulfonamide | | | 1H), 8.06 (s, 1H), 7.88 (d, J = 8.28 Hz, |
| | trifluoroacetate | | | 1H), 7.54 (t, J = 7.91 Hz, 1H), 7.46 (q, |
| | | | | J = 4.68 Hz, 1H), 7.38 (d, J = 7.78 Hz, |
| | | | | 1H), 7.21-7.29 (m, 1H), 7.18 (s, 1H), |
| | | | | 7.09 (d, J = 8.03 Hz, 1 H), 6.65 (dd, J = 2.01, |
| | | | | 8.03 Hz, 1H), 6.22 (s, 1H), |
| | | | | 3.76 (s, 3H), 2.44 (d, J = 4.77 Hz, 3H) |
| 54 | N-(3-{[6-({3- | 1.80^{a} | 413.1 (M + H)⁺ | ¹H NMR (400 MHz, DMSO-d6) δ ppm |
| | [(methylamino)sulfonyl]phenyl}amino)- | | | 9.97 (s, 1H), 9.72 (s, 1H), 9.47 (br. s., |
| | 4- | | | 1H), 8.36 (s, 1H), 8.06 (s, 1H), |
| | pyrimidinyl]amino}phenyl)acetamide | | | 7.88 (dd, J = 1.51, 8.03 Hz, 1H), 7.81 (s, |
| | trifluoroacetate | | | 1H), 7.53 (t, J = 7.91 Hz, 1H), 7.45 (q, |
| | | | | J = 4.85 Hz, 1H), 7.37 (d, J = 7.78 Hz, |
| | | | | 1H), 7.21-7.29 (m, 3H), 6.20 (s, 1H), |
| | | | | 2.44 (d, J = 5.02 Hz, 3H), 2.05 (s, 3H) |
| 55 | N-methyl-3-{[6-(phenylamino)-4- | 1.89^{a} | 356.0 (M + H)⁺ | ¹H NMR (400 MHz, DMSO-d6) δ ppm |
| | pyrimidinyl]amino}benzenesulfonamide | | | 9.75 (br. s., 1H), 9.49 (br. s., 1H), |
| | trifluoroacetate | | | 8.38 (s, 1H), 8.07 (br. s., 1H), 7.87 (d, J = 8.03 Hz, |
| | | | | 1H), 7.53 (d, J = 6.78 Hz, |
| | | | | 3H), 7.46 (d, J = 4.27 Hz, 1H), |
| | | | | 7.29-7.41 (m, 3H), 7.02-7.16 (m, 1H), |
| | | | | 6.99 (s, 1H), 6.20 (s, 1H), 2.44 (d, J = 4.27 Hz, |
| | | | | 3H) |
| 56 | 4-{[6-({3- | 1.81^{a} | 399.1 (M + H)⁺ | ¹H NMR (400 MHz, DMSO-d6) δ ppm |
| | [(methylamino)sulfonyl]phenyl}amino)- | | | 9.71 (s, 1H), 9.64 (s, 1H), 8.42 (s, |
| | 4- | | | 1H), 8.07-8.10 (m, 1H), 7.91 (dd, J = 1.38, |
| | pyrimidinyl]amino}benzamide | | | 8.16 Hz, 1H), 7.84 (d, J = 8.78 Hz, |
| | trifluoroacetate | | | 3H), 7.66 (d, J = 8.78 Hz, 2H), |
| | | | | 7.54 (t, J = 8.03 Hz, 1H), 7.45 (q, J = 4.77 Hz, |
| | | | | 1H), 7.37 (d, J = 7.78 Hz, |
| | | | | 1H), 7.18-7.25 (m, 1H), 6.27 (s, 1H), |
| | | | | 2.45 (d, J = 4.77 Hz, 3H) |
| 57 | 3-({6-[(4-chlorophenyl)amino]-4- | 2.08^{a} | 390.0 (M + H)⁺ | ¹H NMR (400 MHz, DMSO-d6) δ ppm |
| | pyrimidinyl}amino)-N- | | | 9.60 (br. s., 1H), 9.43 (br. s., 1H), |
| | methylbenzenesulfonamide | | | 8.36 (s, 1H), 8.10 (br. s., 1H), 7.91 (d, J = 7.78 Hz, |
| | trifluoroacetate | | | 1H), 7.64 (d, J = 8.28 Hz, |
| | | | | 2H), 7.52 (t, J = 7.78 Hz, 1H), 7.44 (d, |
| | | | | J = 4.52 Hz, 1H), 7.36 (d, J = 7.53 Hz, |
| | | | | 3H), 6.19 (s, 1H), 2.45 (d, J = 4.52 Hz, |
| | | | | 3H) |
| 58 | N-methyl-3-[(6-{[3- | 2.24^{a} | 424.1 (M + H)⁺ | ¹H NMR (400 MHz, DMSO-d6) δ ppm |
| | (trifluoromethyl)phenyl]amino}-4- | | | 9.69 (d, J = 5.52 Hz, 2H), 8.42 (s, 1H), |
| | pyrimidinyl)amino]benzenesulfonamide | | | 8.11 (s, 1H), 8.08 (t, J = 1.76 Hz, 1H), |
| | trifluoroacetate | | | 7.91-7.96 (m, 1H), 7.86 (d, J = 8.78 Hz, |
| | | | | 1H), 7.54 (t, J = 8.03 Hz, 2H), |
| | | | | 7.45 (q, J = 4.94 Hz, 1H), 7.36 (d, J = 8.03 Hz, |
| | | | | 1H), 7.31 (d, J = 7.78 Hz, |
| | | | | 1H), 6.22 (s, 1H), 2.45 (d, J = 4.77 Hz, |
| | | | | 3H) |
| 59 | N-methyl-3-({6-[(2-methyl- | 1.51^{a} | 425.1 (M + H)⁺ | ¹H NMR (400 MHz, DMSO-d6) δ ppm |
| | 1,2,3,4-tetrahydro-7- | | | 9.71 (s, 1H), 9.21 (s, 1H), 8.33 (s, |
| | isoquinolinyl)amino]-4- | | | 1H), 8.08 (s, 1H), 7.84-7.90 (m, 1H), |
| | pyrimidinyl}amino)benzenesulfonamide | | | 7.70-7.79 (m, 1H), 7.48-7.57 (m, |
| | trifluoroaceate | | | 1H), 7.45 (q, J = 4.85 Hz, 1H), |
| | | | | 7.36 (d, J = 7.78 Hz, 1H), 7.27-7.34 (m, |
| | | | | 1H), 7.16-7.25 (m, 2H), 6.12 (s, 1H), |
| | | | | 2.44 (d, J = 5.02 Hz, 3H) |
| 60 | 3-({6-[(2-fluorophenyl)amino]-4- | 1.91^{a} | 374.1 (M + H)⁺ | ¹H NMR (400 MHz, DMSO-d6) δ ppm |
| | pyrimidinyl}amino)-N- | | | 9.71 (s, 1H), 9.21 (s, 1H), 8.33 (s, |
| | methylbenzenesulfonamide | | | 1H), 8.08 (s, 1H), 7.83-7.89 (m, 1H), |
| | trifluoroacetate | | | 7.71-7.78 (m, 1H), 7.49-7.56 (m, |
| | | | | 1H), 7.45 (q, J = 4.85 Hz, 1H), |
| | | | | 7.36 (d, J = 7.78 Hz, 1H), 7.27-7.34 (m, |
| | | | | 1H), 7.17-7.25 (m, 2H), 6.12 (s, 1H), |
| | | | | 2.44 (d, J = 5.02 Hz, 3H) |
| 61 | N-methyl-3-[(6-{[3-(4- | 2.01^{a} | 505.1 (M + H)⁺ | ¹H NMR (400 MHz, DMSO-d6) δ ppm |
| | morpholinylsulfonyl)phenyl]amino}- | | | 9.72 (s, 1H), 9.76 (s, 1H), 8.42 (s, |
| | 4- | | | 1H), 8.05 (s, 1H), 8.09 (s, 1H), |
| | pyrimidinyl)amino]benzenesulfonamide | | | 8.01 (d, J = 8.28 Hz, 1 H), 7.92 (d, J = 7.78 Hz, |
| | trifluoroacetate | | | 1H), 7.50-7.64 (m, 2H), 7.45 (d, |
| | | | | J = 4.02 Hz, 1H), 7.28-7.41 (m, 2H), |
| | | | | 6.23 (s, 1H), 3.66 (m, 4H), 2.91 (m, |
| | | | | 4H), 2.45 (d, J = 3.51 Hz, 3H) |
| 62 | 3-{[6-({3- | 1.96^{a} | 463.1 (M + H)⁺ | ¹H NMR (400 MHz, DMSO-d6) δ ppm |
| | [(ethylamino)sulfonyl]phenyl}amino)- | | | 9.60-9.79 (m, 2H), 8.40 (s, 1H), |
| | 4-pyrimidinyl]amino}-N- | | | 8.09 (m, 2H), 7.91 (t, J = 6.53 Hz, 2H), |
| | methylbenzenesulfonamide | | | 7.48-7.60 (m, 3H), 7.45 (q, J = 4.68 Hz, |
| | trifluoroacetate | | | 1H), 7.29-7.42 (m, 2H), 6.22 (s, |
| | | | | 1H), 2.76-2.90 (m, 2H), 2.45 (d, J = 5.02 Hz, |
| | | | | 3H), 1.00 (t, J = 7.28 Hz, 3H) |
| 63 | N-methyl-3-[(6-{[3- | 1.87^{a} | 434.1 (M + H)⁺ | ¹H NMR (400 MHz, DMSO-d6) δ ppm |
| | (methylsulfonyl)phenyl]amino}- | | | 9.79 (s, 1H), 9.74 (s, 1H), 8.43 (s, |
| | 4- | | | 1H), 8.21 (s, 1H), 8.08 (s, 1H), |
| | pyrimidinyl)amino]benzenesulfonamide | | | 7.99 (d, J = 7.78 Hz, 1H), 7.93 (d, J = 8.03 Hz, |
| | trifluoroacetate | | | 1H), 7.49-7.63 (m, 3H), |
| | | | | 7.43-7.49 (m, 1H), 7.37 (d, J = 7.78 Hz, |
| | | | | 1H), 6.24 (s, 1H), 3.22 (s, 3H), |
| | | | | 2.45 (d, J = 4.52 Hz, 3H) |
| 64 | 3-{[6-(1H-indazol-6-ylamino)-4- | 1.83^{a} | 396.1 (M + H)⁺ | ¹H NMR (400 MHz, DMSO-d6) δ ppm |
| | pyrimidinyl]amino}-N- | | | 9.78 (br. s., 1H), 9.66 (br. s., 1H), |
| | methylbenzenesulfonamide | | | 8.43 (s, 1H), 8.07 (s, 1H), 7.96-8.05 (m, |
| | trifluoroacetate | | | 2H), 7.88 (d, J = 7.78 Hz, 1H), |
| | | | | 7.70 (d, J = 8.78 Hz, 1H), 7.55 (t, J = 7.91 Hz, |
| | | | | 1H), 7.46 (q, J = 4.35 Hz, 1H), |
| | | | | 7.40 (s, 1H), 7.11 (dd, J = 1.76, 8.53 Hz, |
| | | | | 1H), 6.25 (s, 1H), 2.44 (d, J = 4.77 Hz, |
| | | | | 3H) |
| 65 | 3-{[6-({3- | 2.11^{a} | 475.1 (M + H)⁺ | ¹H NMR (400 MHz, DMSO-d6) δ ppm |
| | [(methylamino)sulfonyl]phenyl}amino)- | | | 10.27 (br. s., 1H), 9.78 (br. s., 1H), |
| | 4-pyrimidinyl]amino}-N- | | | 9.68 (br. s., 1H), 8.42 (s, 1H), 8.07 (d, |
| | phenylbenzamide | | | J = 8.28 Hz, 2H), 7.90 (d, J = 8.03 Hz, |
| | trifluoroacetate | | | 1H), 7.85 (d, J = 7.78 Hz, 1H), |
| | | | | 7.79 (d, J = 8.03 Hz, 2H), 7.62 (d, J = 7.28 Hz, |
| | | | | 1H), 7.43-7.60 (m, 3H), |
| | | | | 7.30-7.43 (m, 3H), 7.07-7.17 (m, 1H), |
| | | | | 6.24 (s, 1H), 2.45 (d, J = 4.52 Hz, 3H) |
| 66 | 3-{[6-({3- | 2.03^{a} | 463.0 (M + H)⁺ | ¹H NMR (400 MHz, DMSO-d6) δ ppm |
| | [(dimethylamino)sulfonyl]phenyl}amino)- | | | 9.77 (s, 1H), 9.75 (s, 1H), 8.42 (s, |
| | 4-pyrimidinyl]amino}-N- | | | 1H), 7.98-8.09 (m, 3H), 7.92 (d, J = 8.03 Hz, |
| | methylbenzenesulfonamide | | | 1H), 7.51-7.61 (m, 2H), |
| | trifluoroacetate | | | 7.46 (d, J = 4.77 Hz, 1H), 7.38 (d, J = 7.53 Hz, |
| | | | | 1H), 7.33 (d, J = 7.78 Hz, |
| | | | | 1H), 6.22 (s, 1H), 2.65 (s, 6H), |
| | | | | 2.45 (d, J = 4.52 Hz, 3H) |
| 67 | 3-[(6-{[3- | 1.81^{a} | 435.0 (M + H)⁺ | ¹H NMR (400 MHz, DMSO-d6) δ ppm |
| | (aminosulfonyl)phenyl]amino}-4- | | | 9.69 (s, 1H), 9.67 (s, 1H), 8.40 (s, |
| | pyrimidinyl)amino]-N- | | | 1H), 8.11 (s, 1H), 8.08 (s, 1H), |
| | methylbenzenesulfonamide | | | 7.89-7.95 (m, 1H), 7.86 (d, J = 8.03 Hz, |
| | trifluoroacetate | | | 1H), 7.41-7.57 (m, 4H), |
| | | | | 7.34-7.39 (m, 3H), 6.22 (s, 1H), 2.45 (d, J = 4.77 Hz, |
| | | | | 3H) |
| 68 | 3-{[6-({3- | 2.06^{a} | 477.1 (M + H)⁺ | ¹H NMR (400 MHz, DMSO-d6) δ ppm |
| | [(methylamino)sulfonyl]phenyl}amino)- | | | 9.71 (br. s., 1H), 9.69 (br. s., 1H), |
| | 4-pyrimidinyl]amino}-N-(1- | | | 8.41 (s, 1H), 8.10-8.14 (m, 1H), |
| | methylethyl)benzenesulfonamide | | | 8.06-8.10 (m, 1H), 7.92 (d, J = 7.78 Hz, |
| | trifluoroacetate | | | 1H), 7.88 (d, J = 8.03 Hz, 1H), |
| | | | | 7.60 (d, J = 7.28 Hz, 1H), 7.48-7.57 (m, |
| | | | | 2H), 7.43-7.48 (m, 1H), 7.37 (d, J = 7.78 Hz, |
| | | | | 1H), 7.40 (d, J = 7.78 Hz, |
| | | | | 1H), 6.22 (s, 1H), 3.28 (dq, J = 6.60, |
| | | | | 13.08 Hz, 1H), 2.45 (d, J = 4.77 Hz, |
| | | | | 3H), 0.99 (d, J = 6.27 Hz, 6H) |
| 69 | 3-({6-[(4-acetylphenyl)amino]-4- | 1.99^{a} | 398.0 (M + H)⁺ | ¹H NMR (400 MHz, DMSO-d6) δ ppm |
| | pyrimidinyl}amino)-N- | | | 9.74 (s, 1H), 9.68 (s, 1H), 8.43 (s, |
| | methylbenzenesulfonamide | | | 1H), 8.08-8.14 (m, 1H), |
| | trifluoroacetate | | | 7.90-7.97 (m, 3H), 7.78 (d, J = 9.03 Hz, 2H), |
| | | | | 7.53 (t, J = 7.91 Hz, 1H), 7.45 (d, J = 5.02 Hz, |
| | | | | 1H), 7.36 (d, J = 7.53 Hz, |
| | | | | 1H), 6.30 (s, 1H), 2.52 (s, 3H), |
| | | | | 2.45 (d, J = 5.02 Hz, 3H) |
| 70 | N-methyl-3-[(6-{[4- | 1.94^{a} | 434.0 (M + H)⁺ | ¹H NMR (400 MHz, DMSO-d6) δ ppm |
| | (methylsulfonyl)phenyl]amino}- | | | 9.85 (s, 1H), 9.72 (s, 1H), 8.45 (s, |
| | 4- | | | 1H), 8.09-8.12 (m, 1H), 7.93 (dd, J = 1.76, |
| | pyrimidinyl)amino]benzenesulfonamide | | | 8.03 Hz, 1H), 7.80-7.91 (m, |
| | trifluoroacetate | | | 4H), 7.54 (t, J = 7.91 Hz, 1H), 7.45 (q, |
| | | | | J = 5.02 Hz, 1H), 7.37 (d, J = 7.78 Hz, |
| | | | | 1H), 6.30 (s, 1H), 3.16 (s, 3H), |
| | | | | 2.45 (d, J = 5.02 Hz, 3H) |
| 71 | N-(4-{[6-({3- | 1.76^{a} | 413.1 (M + H)⁺ | ¹H NMR (400 MHz, DMSO-d6) δ ppm |
| | [(methylamino)sulfonyl]phenyl}amino)- | | | 9.94 (s, 1H), 9.75 (br. s., 1H), |
| | 4- | | | 9.43 (br. s., 1H), 8.35 (s, 1H), 8.05 (s, 1H), |
| | pyrimidinyl]amino}phenyl)acetamide | | | 7.85 (d, J = 8.53 Hz, 1H), |
| | trifluoroacetate | | | 7.50-7.60 (m, 3H), 7.46 (q, J = 4.27 Hz, 1H), |
| | | | | 7.36-7.43 (m, 3H), 6.12 (s, 1H), |
| | | | | 2.44 (d, J = 4.02 Hz, 3H), 2.04 (s, 3H) |
| 72 | N-(3-{[6-({3- | 1.88^{a} | 427.1 (M + H)⁺ | ¹H NMR (400 MHz, DMSO-d6) δ ppm |
| | [(methylamino)sulfonyl]phenyl}amino)- | | | 9.90 (s, 1H), 9.75 (s, 1H), 9.49 (br. s., |
| | 4- | | | 1H), 8.36-8.39 (m, 1H), 8.06 (s, 1H), |
| | pyrimidinyl]amino}phenyl)propanamide | | | 7.88 (d, J = 7.78 Hz, 1H), 7.82 (s, 1H), |
| | trifluoroacetate | | | 7.53 (t, J = 7.91 Hz, 1H), 7.45 (q, J = 5.02 Hz, |
| | | | | 1H), 7.38 (d, J = 7.78 Hz, |
| | | | | 1H), 7.22-7.29 (m, 3H), 6.20 (s, 1H), |
| | | | | 2.44 (d, J = 4.77 Hz, 3H), 2.33 (q, J = 7.53 Hz, |
| | | | | 2H), 1.09 (t, J = 7.53 Hz, 3H) |
| 73 | 4-{[6-({3- | 2.14^{a} | 475.1 (M + H)⁺ | ¹H NMR (400 MHz, DMSO-d6) δ ppm |
| | [(methylamino)sulfonyl]phenyl}amino)- | | | 10.08 (s, 1H), 9.66 (br. s., 1H), |
| | 4-pyrimidinyl]amino}-N- | | | 9.65 (br. s., 1H), 8.43 (s, 1H), 8.11 (s, 1H), |
| | phenylbenzamide | | | 7.91-7.97 (m, 3H), 7.75-7.81 (m, |
| | trifluoroacetate | | | 4H), 7.53 (t, J = 7.91 Hz, 1H), 7.45 (q, |
| | | | | J = 4.94 Hz, 1H), 7.32-7.39 (m, 3H), |
| | | | | 7.06-7.13 (m, 1H), 6.29 (s, 1H), |
| | | | | 2.45 (d, J = 5.02 Hz, 3H) |
| 74 | 3-({6-[(1,1-dioxido-2,3-dihydro- | 1.83^{a} | 447.0 (M + H)⁺ | ¹H NMR (400 MHz, DMSO-d6) δ ppm |
| | 1,2-benzisothiazol-6-yl)amino]- | | | 9.76 (s, 1H), 9.72 (s, 1H), 8.46 (s, |
| | 4-pyrimidinyl}amino)-N- | | | 1H), 8.33 (s, 1H), 8.09 (s, 1H), |
| | methylbenzenesulfonamide | | | 7.93 (d, J = 8.03 Hz, 1H), 7.81 (br. s., 1H), |
| | trifluoroacetate | | | 7.65-7.71 (m, 1H), 7.54 (t, J = 8.03 Hz, |
| | | | | 1H), 7.43-7.51 (m, 2H), 7.37 (d, |
| | | | | J = 7.78 Hz, 1H), 6.23 (s, 1H), 4.35 (s, |
| | | | | 2H), 2.45 (d, J = 4.77 Hz, 3H) |
| 75 | N-methyl-3-({6-[(2-oxo-2,3- | 1.76^{a} | 411.0 (M + H)⁺ | ¹H NMR (400 MHz, DMSO-d6) δ ppm |
| | dihydro-1H-indol-6-yl)amino]-4- | | | 10.41 (br. s., 1H), 9.75 (br. s., 1H), |
| | pyrimidinyl}amino)benzenesulfonamide | | | 9.48 (br. s., 1H), 8.38 (br. s., 1H), |
| | trifluoroacetate | | | 8.06 (br. s., 1H), 7.87 (d, J = 7.53 Hz, 1H), |
| | | | | 7.54 (t, J = 7.40 Hz, 1H), |
| | | | | 7.42-7.50 (m, 1H), 7.38 (d, J = 7.03 Hz, 1H), |
| | | | | 7.20 (br. s., 1H), 7.16 (d, J = 7.28 Hz, |
| | | | | 1H), 7.01 (d, J = 6.78 Hz, 1H), |
| | | | | 6.18 (br. s., 1H), 3.44 (br. s., 2H), |
| | | | | 2.42-2.48 (m, J = 3.51 Hz, 3H) |
| 76 | N-methyl-3-({6-[(2-methyl-1,3- | 1.98^{a} | 427.0 (M + H)⁺ | ¹H NMR (400 MHz, DMSO-d6) δ ppm |
| | benzothiazol-5-yl)amino]-4- | | | 9.79 (br. s., 1H), 9.68 (br. s., 1H), |
| | pyrimidinyl}amino)benzenesulfonamide | | | 8.42 (s, 1H), 8.21 (s, 1H), 8.08 (br. s., 1H), |
| | trifluoroacetate | | | 7.97 (d, J = 8.5 Hz, 1H), 7.88 (d, J = 7.8 Hz, |
| | | | | 1H), 7.48-7.58 (m, 2H), |
| | | | | 7.46 (d, J = 4.5 Hz, 1H), 7.39 (d, J = 7.8 Hz, |
| | | | | 1H), 6.25 (s, 1H), 3.18 (s, 1H), |
| | | | | 2.80 (s, 3H), 2.45 (d, J = 4.5 Hz, 3H) |
| 77 | N-methyl-3-({6-[(3- | 2.17^{a} | 401.0 (M + H)⁺ | ¹H NMR (400 MHz, DMSO-d6) δ ppm |
| | nitrophenyl)amino]-4- | | | 9.87 (br. s., 1H), 9.74 (br. s., 1H), |
| | pyrimidinyl}amino)benzenesulfonamide | | | 8.71 (br. s., 1H), 8.45 (br. s., 1H), 8.08 (br. |
| | trifluoroacetate | | | s., 1H), 7.99 (d, J = 7.53 Hz, 1H), |
| | | | | 7.93 (d, J = 7.53 Hz, 1H), 7.81 (d, J = 7.78 Hz, |
| | | | | 1H), 7.49-7.63 (m, 2H), |
| | | | | 7.41-7.49 (m, 1H), 7.36 (d, J = 7.28 Hz, |
| | | | | 1H), 6.25 (br. s., 1H), 2.44 (d, J = 2.51 Hz, |
| | | | | 3H) |
| 78 | N-methyl-3-[(6-{[4-(4- | 1.85^{a} | 469.1 (M + H)⁺ | ¹H NMR (400 MHz, DMSO-d6) δ ppm |
| | morpholinylcarbonyl)phenyl]amino}- | | | 9.61 (br. s., 1H), 9.52 (br. s., 1H), |
| | 4- | | | 8.38 (s, 1H), 8.11 (s, 1H), 7.92 (d, J = 8.28 Hz, |
| | pyrimidinyl)amino]benzenesulfonamide | | | 1H), 7.69 (s, 1H), 7.67 (s, 1H), |
| | | | | 7.52 (t, J = 8.03 Hz, 1H), |
| | | | | 7.31-7.41 (m, 4H), 6.25 (s, 1H), 3.61 (m, 4H), |
| | | | | 3.52 (m, 4H), 2.45 (s, 3H) |
| 79 | N-methyl-4-{[6-({3- | 1.76^{a} | 413.0 (M + H)⁺ | ¹H NMR (400 MHz, DMSO-d6) δ ppm |
| | [(methylamino)sulfonyl]phenyl}amino)- | | | 9.76 (br. s., 1H), 9.69 (br. s., 1H), |
| | 4- | | | 8.43 (s, 1H), 8.30 (d, J = 3.76 Hz, 1H), |
| | pyrimidinyl]amino}benzamide | | | 8.08 (br. s., 1H), 7.90 (d, J = 7.53 Hz, 1H), |
| | trifluoroacetate | | | 7.82 (br. s., 1H), 7.80 (br. s., 1H), |
| | | | | 7.67 (br. s., 1H), 7.65 (br. s., 1H), 7.55 (t, J = 7.91 Hz, |
| | | | | 1H), 7.46 (d, J = 4.52 Hz, |
| | | | | 1H), 7.38 (d, J = 7.53 Hz, 1H), 6.26 (s, |
| | | | | 1H), 2.78 (d, J = 3.76 Hz, 3H), |
| | | | | 2.45 (d, J = 4.52 Hz, 3H) |
| 80 | 3-{[6-(2,3-dihydro-1,4- | 1.96^{a} | 414.0 (M + H)⁺ | ¹H NMR (400 MHz, DMSO-d6) δ ppm |
| | benzodioxin-6-ylamino)-4- | | | 9.96 (br. s., 1H), 9.58 (br. s., 1H), |
| | pyrimidinyl]amino}-N- | | | 8.37 (s, 1H), 8.03 (s, 1H), 7.82 (d, J = 7.78 Hz, |
| | methylbenzenesulfonamide | | | 1H), 7.55 (t, J = 7.91 Hz, 1H), |
| | trifluoroacetate | | | 7.49 (d, J = 5.02 Hz, 1H), 7.41 (d, J = 7.78 Hz, |
| | | | | 1H), 7.05 (s, 1H), |
| | | | | 6.84-6.91 (m, 2H), 6.12 (s, 1H), 4.25 (br. s., 4H), |
| | | | | 2.44 (d, J = 4.77 Hz, 3H) |
| 81 | N-methyl-3-[(6-{[4- | 1.97^{a} | 386.1 (M + H)⁺ | ¹H NMR (400 MHz, DMSO-d6) δ ppm |
| | (methyloxy)phenyl]amino}-4- | | | 10.28 (br. s., 1H), 9.96 (br. s., 1H), |
| | pyrimidinyl)amino]benzenesulfonamide | | | 8.42 (s, 1H), 7.99 (s, 1H), 7.77 (d, J = 8.03 Hz, |
| | hydrochloride | | | 1H), 7.51-7.62 (m, 2H), |
| | | | | 7.47 (d, J = 7.78 Hz, 1H), 7.35 (d, J = 8.78 Hz, |
| | | | | 2H), 7.01 (d, J = 8.78 Hz, |
| | | | | 2H), 6.12 (s, 1H), 2.43 (d, J = 4.77 Hz, |
| | | | | 3H) |
| 82 | N-methyl-3-[(6-{[4-(4- | 1.87^{a} | 441.1 (M + H)⁺ | ¹H NMR (400 MHz, DMSO-d6) δ ppm |
| | morpholinyl)phenyl]amino}-4- | | | 9.45 (s, 1H), 8.96 (s, 1H), 8.26 (s, |
| | pyrimidinyl)amino]benzenesulfonamide | | | 1H), 8.11 (t, J = 1.63 Hz, 1H), |
| | hydrochloride | | | 7.85-7.91 (m, 1H), 7.49 (t, J = 7.91 Hz, |
| | | | | 1H), 7.42 (q, J = 5.02 Hz, 1H), 7.35 (s, |
| | | | | 1H), 7.33 (s, 1H), 7.30 (d, J = 8.03 Hz, |
| | | | | 1H), 6.95 (s, 1H), 6.93 (s, 1H), |
| | | | | 6.06 (s, 1H), 3.72-3.78 (m, 4H), |
| | | | | 3.03-3.09 (m, 4H), 2.44 (d, J = 5.02 Hz, |
| | | | | 3H) |
| 83 | 3-[(6-{[4-(1,1- | 2.25^{a} | 412.1 (M + H)⁺ | ¹H NMR (400 MHz, DMSO-d6) δ ppm |
| | dimethylethyl)phenyl]amino}-4- | | | 9.78 (br. s., 1H), 9.46 (br. s., 1H), |
| | pyrimidinyl)amino]-N- | | | 8.36 (s, 1H), 8.06 (s, 1H), 7.85 (d, J = 7.78 Hz, |
| | methylbenzenesulfonamide | | | 1H), 7.54 (t, J = 7.91 Hz, 1H), |
| | trifluoroacetate | | | 7.46 (q, J = 4.68 Hz, 1H), |
| | | | | 7.35-7.43 (m, 5H), 6.17 (s, 1H), 2.44 (d, J = 4.77 Hz, |
| | | | | 3H), 1.29 (s, 9H) |
| 84 | N-methyl-3-[(6-{[3-(4- | 1.96^{a} | 441.1 (M + H)⁺ | ¹H NMR (400 MHz, DMSO-d6) δ ppm |
| | morpholinyl)phenyl]amino}-4- | | | 9.52 (s, 1H), 9.11 (s, 1H), 8.31 (s, |
| | pyrimidinyl)amino]benzenesulfonamide | | | 1H), 8.06-8.10 (m, 1H), |
| | | | | 7.89-7.95 (m, 1H), 7.50 (t, J = 8.03 Hz, 1H), |
| | | | | 7.43 (q, J = 5.02 Hz, 1H), 7.32 (d, J = 7.78 Hz, |
| | | | | 1H), 7.12-7.20 (m, 1H), |
| | | | | 7.06-7.08 (m, 1H), 7.03 (d, J = 7.78 Hz, |
| | | | | 1H), 6.63 (dd, J = 2.01, 8.28 Hz, |
| | | | | 1H), 6.20 (s, 1H), 3.72-3.79 (m, 4H), |
| | | | | 3.06-3.12 (m, 4H), 2.44 (d, J = 5.02 Hz, |
| | | | | 3H) |
| 85 | 3-({6-[(3-bromo-5- | 2.24^{a} | 449.0 (M + H)⁺ | ¹H NMR (400 MHz, DMSO-d6) δ ppm |
| | methylphenyl)amino]-4- | | | 10.04 (br. s., 1H), 9.87 (br. s., 1H), |
| | pyrimidinyl}amino)-N- | | | 8.46 (s, 1H), 8.03 (br. s., 1H), 7.87 (d, |
| | methylbenzenesulfonamide | | | J = 7.53 Hz, 1H), 7.74 (br. s., 1H), |
| | hydrochloride | | | 7.47-7.61 (m, 2H), 7.43 (d, J = 8.03 Hz, |
| | | | | 1H), 7.31 (s, 1H), 7.10 (s, 1H), |
| | | | | 6.28 (s, 1H), 2.45 (d, J = 4.52 Hz, 3H), |
| | | | | 2.31 (s, 3H) |
| 86 | 3-[(6-{[4- | 1.66^{a} | 399.1 (M + H)⁺ | ¹H NMR (400 MHz, DMSO-d6) δ ppm |
| | (dimethylamino)phenyl]amino}- | | | 9.42 (s, 1H), 8.83 (s, 1H), 8.23 (s, |
| | 4-pyrimidinyl)amino]-N- | | | 1H), 8.11 (s, 1H), 7.83-7.89 (m, 1H), |
| | methylbenzenesulfonamide | | | 7.48 (t, J = 8.03 Hz, 1H), 7.41 (q, J = 4.94 Hz, |
| | | | | 1H), 7.29 (d, J = 7.78 Hz, |
| | | | | 1H), 7.26 (s, 1H), 7.24 (s, 1H), |
| | | | | 6.77 (s, 1H), 6.75 (s, 1H), 5.99 (s, 1H), |
| | | | | 2.88 (s, 6H), 2.43 (d, J = 5.02 Hz, 3H) |
| 87 | 3-[(6-{[3- | 1.68^{a} | 399.1 (M + H)⁺ | ¹H NMR (400 MHz, DMSO-d6) δ ppm |
| | (dimethylamino)phenyl]amino}- | | | 9.88 (br. s., 1H), 9.55 (br. s., 1H), |
| | 4-pyrimidinyl)amino]-N- | | | 8.38 (s, 1H), 8.03 (s, 1H), 7.85 (d, J = 8.03 Hz, |
| | methylbenzenesulfonamide | | | 1H), 7.55 (t, J = 7.91 Hz, 1H), |
| | trifluoroacetate | | | 7.47 (q, J = 4.77 Hz, 1H), 7.41 (d, J = 7.78 Hz, |
| | | | | 1H), 7.18-7.26 (m, 1H), |
| | | | | 6.84-6.97 (m, 2H), 6.58-6.67 (m, |
| | | | | 1H), 6.21 (s, 1H), 2.95 (s, 6H), |
| | | | | 2.42-2.47 (m, 3H) |
| 88 | methyl 4-{[6-({3- | 2.12^{a} | 414.0 (M + H)⁺ | ¹H NMR (400 MHz, DMSO-d6) δ ppm |
| | [(methylamino)sulfonyl]phenyl}amino)- | | | 9.74 (s, 1H), 9.67 (s, 1H), 8.43 (s, |
| | 4- | | | 1H), 8.09-8.13 (m, 1H), |
| | pyrimidinyl]amino}benzoate | | | 7.87-7.96 (m, 3H), 7.80 (d, J = 8.78 Hz, 2H), |
| | | | | 7.53 (t, J = 7.91 Hz, 1H), |
| | | | | 7.42-7.49 (m, 1H), 7.35 (d, J = 7.78 Hz, 1H), |
| | | | | 6.30 (s, 1H), 3.83 (s, 3H), 2.45 (d, J = 4.27 Hz, |
| | | | | 3H) |
| 89 | 1-methylethyl 4-{[6-({3- | 2.28^{a} | 442.1 (M + H)⁺ | ¹H NMR (400 MHz, DMSO-d6) δ ppm |
| | [(methylamino)sulfonyl]phenyl}amino)- | | | 9.75 (s, 1H), 9.70 (s, 1H), 8.43 (s, |
| | 4- | | | 1H), 8.08-8.11 (m, 1H), |
| | pyrimidinyl]amino}benzoate | | | 7.86-7.95 (m, 3H), 7.75-7.80 (m, 2H), 7.54 (t, J = 7.91 Hz, |
| | trifluoroacetate | | | 1H), 7.45 (d, J = 5.02 Hz, |
| | | | | 1H), 7.36 (d, J = 8.03 Hz, 1H), 6.29 (s, |
| | | | | 1H), 5.11 (quin, J = 6.27 Hz, 1H), |
| | | | | 2.45 (d, J = 5.02 Hz, 3H), 1.32 (d, J = 6.27 Hz, |
| | | | | 6H) |
| 90 | 3-({6-[(4-chloro-3- | 2.21^{a} | 404.0 (M + H)⁺ | ¹H NMR (400 MHz, DMSO-d6) δ ppm |
| | methylphenyl)amino]-4- | | | 10.46 (br. s., 1H), 10.31 (br. s., 1H), |
| | pyrimidinyl}amino)-N- | | | 8.48 (s, 1H), 7.99 (s, 1H), 7.79 (d, J = 8.06 Hz, |
| | methylbenzenesulfonamide | | | 1H), 7.54-7.63 (m, 2H), |
| | hydrochloride | | | 7.46-7.53 (m, 2H), 7.35-7.46 (m, |
| | | | | 2H), 6.35 (s, 1H), 2.44 (d, J = 3.27 Hz, |
| | | | | 3H), 2.34 (s, 3H) |
| 91 | 3-({6-[(4-fluoro-3- | 2.12^{a} | 388.1 (M + H)⁺ | ¹H NMR (400 MHz, DMSO-d6) δ ppm |
| | methylphenyl)amino]-4- | | | 10.52 (br. s., 1H), 10.29 (br. s., 1H), |
| | pyrimidinyl}amino)-N- | | | 8.47 (s, 1H), 7.98 (s, 1H), 7.78 (d, J = 8.03 Hz, |
| | methylbenzenesulfonamide | | | 1H), 7.55-7.65 (m, 2H), |
| | hydrochloride | | | 7.50 (d, J = 7.78 Hz, 1H), 7.38 (dd, J = 2.26, |
| | | | | 6.78 Hz, 1H), 7.30 (dt, J = 3.92, |
| | | | | 7.47 Hz, 1H), 7.17-7.25 (m, |
| | | | | 1H), 6.28 (s, 1H), 2.44 (d, J = 4.27 Hz, |
| | | | | 3H), 2.26 (s, 3H) |
| 92 | 3-{[6-(1H-indol-6-ylamino)-4- | 2.05^{a} | 395.1 (M + H)⁺ | ¹H NMR (400 MHz, DMSO-d6) δ ppm |
| | pyrimidinyl]amino}-N- | | | 11.01 (br. s., 1H), 9.45 (s, 1H), |
| | methylbenzenesulfonamide | | | 9.09 (s, 1H), 8.30 (s, 1H), 8.10-8.14 (m, |
| | | | | 1H), 7.88 (dd, J = 1.38, 8.16 Hz, 1H), |
| | | | | 7.72 (s, 1H), 7.45-7.52 (m, 2H), |
| | | | | 7.38-7.45 (m, 1H), 7.30 (d, J = 7.78 Hz, |
| | | | | 1H), 7.27 (t, J = 2.64 Hz, 1H), |
| | | | | 7.00 (dd, J = 1.76, 8.53 Hz, 1H), 6.38 (br. |
| | | | | s., 1H), 6.14 (s, 1H), 2.41-2.47 (m, |
| | | | | 3H) |
| 93 | N-methyl-3-{[6-({3- | 1.80^{a} | 448.9 (M + H)⁺ | ¹H NMR (400 MHz, DMSO-d6) δ ppm |
| | [(methylsulfonyl)amino]phenyl}amino)- | | | 9.75 (s, 1H), 9.56 (s, 1H), 9.34 (s, |
| | 4- | | | 1H), 8.34 (s, 1H), 8.10 (t, J = 1.76 Hz, |
| | pyrimidinyl]amino}benzenesulfonamide | | | 1H), 7.88-7.94 (m, 1H), 7.51 (t, J = 8.03 Hz, |
| | | | | 1H), 7.43-7.47 (m, 1H), |
| | | | | 7.41-7.43 (m, 2H), 7.33 (d, J = 7.78 Hz, |
| | | | | 1H), 7.25 (t, J = 7.91 Hz, 1H), |
| | | | | 6.80-6.86 (m, 1H), 6.20 (s, 1H), |
| | | | | 3.01 (s, 3H), 2.44 (d, J = 5.02 Hz, 3H) |
| 94 | N-methyl-3-({6-[(3-methyl-1H- | 1.83^{a} | 409.9 (M + H)⁺ | ¹H NMR (400 MHz, DMSO-d6) δ ppm |
| | indazol-6-yl)amino]-4- | | | 12.40 (s, 1H), 9.57 (s, 1H), 9.39 (s, |
| | pyrimidinyl}amino)benzenesulfonamide | | | 1H), 8.39 (s, 1H), 8.10-8.14 (m, 1H), |
| | | | | 8.03 (s, 1H), 7.92 (dd, J = 1.51, 8.03 Hz, |
| | | | | 1H), 7.59 (d, J = 8.53 Hz, 1H), |
| | | | | 7.52 (t, J = 7.91 Hz, 1H), 7.44 (q, J = 4.94 Hz, |
| | | | | 1H), 7.33 (d, J = 7.78 Hz, |
| | | | | 1H), 7.07 (dd, J = 1.51, 8.78 Hz, 1H), |
| | | | | 6.24 (s, 1H), 2.42-2.47 (m, 6H) |
| 95 | 3-({6-[(4-{[2- | 1.62^{a} | 471.0 (M + H)⁺ | ¹H NMR (400 MHz, DMSO-d6) δ ppm |
| | (diethylamino)ethyl]oxy}phenyl)amino]- | | | 11.00 (br. s., 1H), 10.71 (br. s., 2H), |
| | 4-pyrimidinyl}amino)-N- | | | 8.49 (s, 1H), 7.91 (br. s., 1H), 7.70 (d, |
| | methylbenzenesulfonamide | | | J = 7.28 Hz, 1H), 7.60 (t, J = 7.78 Hz, |
| | | | | 1H), 7.53 (br. s., 1H), 7.34 (d, J = 8.28 Hz, |
| | | | | 2H), 7.07 (d, J = 8.28 Hz, 2H), |
| | | | | 6.33 (br. s., 1H), 4.40 (br. s., 2H), |
| | | | | 3.48 (br. s., 2H), 3.08-3.29 (m, 4H), |
| | | | | 2.39 (s, 3H), 1.24 (t, J = 6.90 Hz, 6H) |
| 96 | 1-methylethyl [(3-{[6-({3- | 6.52^{b} | 472.1 (M + H)⁺ | ¹H NMR (400 MHz, DMSO-d6) δ ppm |
| | [(methylamino)sulfonyl]phenyl}amino)- | | | 9.70 (br. s., 1H), 9.42 (br. s., 1H), |
| | 4- | | | 8.37 (s, 1H), 8.05-8.11 (m, 1H), |
| | pyrimidinyl]amino}phenyl)oxy]acetate | | | 7.86-7.94 (m, 1H), 7.53 (t, J = 7.91 Hz, |
| | trifluoroacetate | | | 1H), 7.45 (q, J = 4.77 Hz, 1H), |
| | | | | 7.36 (d, J = 7.78 Hz, 1H), 7.26-7.30 (m, |
| | | | | 1H), 7.23 (t, J = 8.03 Hz, 1H), |
| | | | | 7.08-7.14 (m, 1H), 6.54-6.62 (m, 1H), |
| | | | | 6.21 (s, 1H), 5.01 (quin, J = 6.27 Hz, |
| | | | | 1H), 4.72 (s, 2H), 2.44 (d, J = 4.77 Hz, |
| | | | | 3H), 1.23 (s, 3H), 1.22 (s, 3H) |
| 97 | 3-{[6-(1,3-benzothiazol-6- | 5.20^{b} | 413.1 (M + H)⁺ | ¹H NMR (400 MHz, DMSO-d6) δ ppm |
| | ylamino)-4-pyrimidinyl]amino}-N- | | | 9.73 (s, 1H), 9.76 (s, 1H), 9.27 (s, |
| | methylbenzenesulfonamide | | | 1H), 8.50 (d, J = 2.01 Hz, 1H), 8.43 (s, |
| | trifluoroacetate | | | 1H), 8.07-8.10 (m, 1H), 8.04 (d, J = 8.78 Hz, |
| | | | | 1H), 7.89 (dd, J = 1.63, 7.91 Hz, |
| | | | | 1H), 7.59 (dd, J = 2.01, 8.78 Hz, |
| | | | | 1H), 7.54 (t, J = 8.03 Hz, 1H), |
| | | | | 7.43-7.49 (m, 1H), 7.38 (d, J = 7.53 Hz, |
| | | | | 1H), 6.24 (s, 1H), 2.44 (d, J = 5.02 Hz, |
| | | | | 3H) |
| 98 | 3-{[6-(1H-indol-5-ylamino)-4- | 5.45^{b} | 395.1 (M + H)⁺ | ¹H NMR (400 MHz, DMSO-d6) δ ppm |
| | pyrimidinyl]amino}-N- | | | 11.05 (br. s., 1 H), 9.38 (s, 1 H), |
| | methylbenzenesulfonamide | | | 8.91 (s, 1 H), 8.25 (s, 1 H), 8.08-8.14 (m, |
| | trifluoroacetate | | | 1 H), 7.86 (d, J = 7.55 Hz, 1 H), 7.60 (s, |
| | | | | 1 H), 7.46 (t, J = 7.93 Hz, 1 H), |
| | | | | 7.33-7.40 (m, 3 H), 7.28 (d, J = 7.55 Hz, 1 |
| | | | | H), 7.05-7.12 (m, 1 H), 6.40 (br. s., 1 |
| | | | | H), 6.04 (s, 1 H), 2.42 (d, J = 5.04 Hz, 3 |
| | | | | H) |
| 99 | 3-{[6-(1,3-benzothiazol-5- | 5.34^{b} | 413 (M + H)⁺ | ¹H NMR (400 MHz, DMSO-d6) δ ppm |
| | ylamino)-4-pyrimidinyl]amino}-N- | | | 9.77 (s, 1 H), 9.71 (br. s., 1 H), |
| | methylbenzenesulfonamide | | | 9.39 (s, 1 H), 8.43 (s, 2 H), 8.11 (d, J = 8.56 Hz, |
| | trifluoroacetate | | | 1 H), 8.08 (s, 1 H), |
| | | | | 7.89 (m, 1 H), 7.58 (dd, J = 8.56, 2.01 Hz, 1 |
| | | | | H), 7.54 (t, J = 8.06 Hz, 1 H), 7.46 (q, |
| | | | | J = 5.04 Hz, 1 H), 7.38 (d, J = 7.81 Hz, 1 |
| | | | | H), 6.26 (s, 1 H), 2.44 (d, J = 4.78 Hz, 3 |
| | | | | H) |
| 100 | 3-({6-[(3-fluoro-4- | 1.05^{d} | 388.1 (M + H)⁺ | ¹H NMR (400 MHz, DMSO-d6) δ ppm |
| | methylphenyl)amino]-4- | | | 9.72 (s, 1 H), 9.52 (s, 1 H), 8.33 (s, 1 |
| | pyrimidinyl}amino)-N- | | | H), 7.97-8.04 (m, 1 H), 7.79 (dd, |
| | methylbenzenesulfonamide | | | J = 8.05, 1.21 Hz, 1 H), 7.47-7.53 (m, |
| | trifluoroacetate | | | 1 H), 7.39-7.46 (m, 2 H), 7.35 (d, |
| | | | | J = 8.16 Hz, 1 H), 7.17 (dd, J = 8.38, |
| | | | | 8.60 Hz, 1 H), 7.12 (dd, J = 8.16, 1.98 Hz, |
| | | | | 1 H), 6.14 (s, 1 H), 2.41 (d, J = 4.85 Hz, |
| | | | | 3 H), 2.15 (s, 3 H) |
| 101 | 3-({6-[(3-fluorophenyl)amino]-4- | 1.01^{d} | 374.2 (M + H)⁺ | ¹H NMR (400 MHz, DMSO-d6) δ ppm |
| | pyrimidinyl}amino)-N- | | | 9.77 (s, 1 H), 9.66 (s, 1 H), 8.40 (s, 1 |
| | methylbenzenesulfonamide | | | H), 8.05 (s, 1 H), 7.87 (dd, J = 8.16, |
| | trifluoroacetate | | | 1.10 Hz, 1 H), 7.58-7.65 (m, 1 H), |
| | | | | 7.52 (t, J = 7.94 Hz, 1 H), 7.44 (q, |
| | | | | J = 5.07 Hz, 1 H), 7.36 (d, J = 7.94 Hz, 1 |
| | | | | H), 7.32 (m, 1 H), 7.23-7.28 (m, 1 H), |
| | | | | 6.76-6.83 (m, 1 H), 6.21 (s, 1 H), |
| | | | | 2.42 (d, J = 4.63 Hz, 3 H) |
| 102 | 3-[(6-{[3-fluoro-4- | 1.27^{d} | 442.2 (M + H)⁺ | ¹H NMR (400 MHz, DMSO-d6) δ ppm |
| | (trifluoromethyl)phenyl]amino}-4- | | | 9.98 (s, 1 H), 9.75 (s, 1 H), 8.45 (s, 1 |
| | pyrimidinyl)amino]-N- | | | H), 8.10 (s, 1 H), 8.03 (d, J = 14.56 Hz, |
| | methylbenzenesulfonamide | | | 1 H), 7.92 (d, J = 8.03 Hz, 1 H), |
| | trifluoroacetamide | | | 7.60-7.67 (m, 1 H), 7.44-7.54 (m, 3 H), |
| | | | | 7.35 (d, J = 7.78 Hz, 1 H), 6.29 (s, 1 H), |
| | | | | 2.43 (d, J = 4.77 Hz, 3 H) |
| 103 | N-methyl-3-[(6-{[4-(methyloxy)- | 1.07^{d} | 454.2 (M + H)⁺ | ¹H NMR (400 MHz, DMSO-d6) δ ppm |
| | 3-(trifluoromethyl)phenyl]amino}- | | | 9.55 (s, 1 H), 9.31 (s, 1 H), 8.30 (s, 1 |
| | 4- | | | H), 8.04 (t, J = 1.8 Hz, 1 H), 7.86 (dd, |
| | pyrimidinyl)amino]benzenesulfonamide | | | J = 7.9, 1.8 Hz, 1 H), 7.83 (d, J = 2.7 Hz, |
| | trifluoroacetate | | | 1 H), 7.75 (dd, J = 9.0, 2.7 Hz, 1 H), |
| | | | | 7.47 (t, J = 8.1 Hz, 1 H), 7.40 (q, J = 5.1 Hz, |
| | | | | 1 H), 7.30 (d, J = 7.7 Hz, 1 H), |
| | | | | 7.21 (d, J = 9.3 Hz, 1 H), 6.06 (s, 1 H), |
| | | | | 3.82 (s, 3 H), 2.40 (d, J = 5.1 Hz, 3 H) |
| 104 | 3-({6-[(4-chloro-3- | 1.14^{d} | 408.2 (M + H)⁺ | ¹H NMR (400 MHz, DMSO-d6) δ ppm |
| | fluorophenyl)amino]-4- | | | 9.62 (d, J = 7.28 Hz, 2 H), 8.39 (s, 1 H), |
| | pyrimidinyl}amino)-N- | | | 8.08 (t, J = 1.76 Hz, 1 H), |
| | methylbenzenesulfonamide | | | 7.89-7.96 (m, 2 H), 7.40-7.52 (m, 3 H), |
| | trifluoroacetate | | | 7.29-7.35 (m, 2 H), 6.19 (s, 1 H), 2.42 (d, |
| | | | | J = 5.07 Hz, 3 H) |
| 105 | 3-[(6-{[3-fluoro-4- | 0.97^{d} | 404.2 (M + H)⁺ | ¹H NMR (400 MHz, DMSO-d6) δ ppm |
| | (methyloxy)phenyl]amino}-4- | | | 9.84 (s, 1 H), 9.56 (br. s., 1 H), |
| | pyrimidinyl)amino]-N- | | | 8.33 (s, 1 H), 8.00 (s, 1 H), 7.73-7.80 (m, |
| | methylbenzenesulfonamide | | | 1 H), 7.42-7.54 (m, 3 H), 7.38 (d, |
| | trifluoroacetate | | | J = 7.50 Hz, 1 H), 7.10-7.17 (m, 2 H), |
| | | | | 6.07 (s, 1 H), 3.79 (s, 3 H), 2.40 (d, |
| | | | | J = 4.41 Hz, 3 H) |
| 106 | N-methyl-3-[(6-{[4-methyl-3- | 1.15^{d} | 438.2 (M + H)⁺ | ¹H NMR (400 MHz, DMSO-d6) δ ppm |
| | (trifluoromethyl)phenyl]amino}-4- | | | 9.68 (s, 1 H), 9.57 (s, 1 H), 8.37 (s, 1 |
| | pyrimidinyl)amino]benzenesulfonamide | | | H), 8.05 (s, 1 H), 7.93 (s, 1 H), |
| | trifluoroacetate | | | 7.88 (d, J = 7.06 Hz, 1 H), 7.73 (d, J = 7.06 Hz, |
| | | | | 1 H), 7.51 (t, J = 7.94 Hz, 1 H), |
| | | | | 7.43 (q, J = 4.85 Hz, 1 H), 7.34 (d, |
| | | | | J = 8.16 Hz, 2 H), 6.16 (s, 1 H), 2.42 (d, |
| | | | | J = 4.85 Hz, 3 H), 2.36 (br. s., 3 H) |
| 107 | 3-[(6-{[4-chloro-3- | 1.24^{d} | 458.2 (M + H)⁺ | ¹H NMR (400 MHz, DMSO-d6) δ ppm |
| | (trifluoromethyl)phenyl]amino}-4- | | | 9.73 (s, 1 H), 9.66 (s, 1 H), 8.40 (s, 1 |
| | pyrimidinyl)amino]-N- | | | H), 8.20 (d, J = 2.65 Hz, 1 H), 8.07 (t, |
| | methylbenzenesulfonamide | | | J = 1.76 Hz, 1 H), 7.89-7.96 (m, 2 H), |
| | trifluoroacetate | | | 7.60 (d, J = 8.82 Hz, 1 H), 7.50 (t, |
| | | | | J = 7.94 Hz, 1 H), 7.43 (q, J = 4.85 Hz, 1 |
| | | | | H), 7.33 (d, J = 8.38 Hz, 1 H), 6.19 (s, 1 |
| | | | | H), 2.42 (d, J = 5.07 Hz, 3 H) |
| 108 | N-methyl-3-[(6-{[4-(2,2,2- | 2.18^{a} | 438.1 (M + H)⁺ | ¹H NMR (400 MHz, DMSO-d6) δ ppm |
| | trifluoroethyl)phenyl]amino}-4- | | | 9.69 (s, 1 H), 9.46 (s, 1 H), 8.37 (s, 1 |
| | pyrimidinyl)amino]benzenesulfonamide | | | H), 8.08 (s, 1 H), 7.85-7.93 (m, 1 H), |
| | trifluoroacetate | | | 7.50-7.57 (m, 3 H), 7.45 (q, J = 4.94 Hz, |
| | | | | 1 H), 7.37 (d, J = 8.03 Hz, 1 H), |
| | | | | 7.32 (d, J = 8.28 Hz, 2 H), 6.21 (s, 1 H), |
| | | | | 3.57-3.64 (q, J = 11.5 Hz, 2 H), |
| | | | | 2.45 (d, J = 5.02 Hz, 3 H) |
| 109 | N-methyl-4-(methylthio)-3-({6- | 1.83^{a} | 471.0 (M + H)⁺ | ¹H NMR (400 MHz, DMSO-d₆) δ ppm |
| | [(2-oxo-1,2,3,4-tetrahydro-7- | | | 2.39-2.45 (m, 5 H) 2.49 (s, 3 H) |
| | quinolinyl)amino]-4- | | | 2.77-2.82 (m, 2 H) 4.94-4.97 (m, 0 H) |
| | pyrimidinyl}amino)benzenesulfonamide | | | 5.84-5.86 (m, 1 H) 7.03-7.13 (m, 3 |
| | | | | H) 7.43-7.51 (m, 2 H) 7.58-7.62 (m, |
| | | | | 1 H) 7.67-7.68 (m, 1 H) |
| | | | | 8.14-8.16 (m, 1 H) 8.69-8.72 (m, 1 H) |
| | | | | 9.09-9.11 (m, 1 H) 10.02-10.12 (m, 1 H) |
| 110 | 4-[(6-{[5-[(methylamino)sulfonyl]- | 1.93^{a} | 446.0 (M + H)⁺ | ¹H NMR (400 MHz, DMSO-d₆) δ ppm |
| | 2-(methylthio)phenyl]amino}-4- | | | 2.44 (d, J = 5.02 Hz, 3 H) 5.92 (s, 1 H) |
| | pyrimidinyl)amino]benzoic acid | | | 7.47 (q, J = 5.02 Hz, 1 H) 7.52 (d, |
| | trifluoroacetate | | | J = 8.53 Hz, 1 H) 7.62-7.69 (m, 2 H) |
| | | | | 7.71 (d, J = 8.78 Hz, 2 H) 7.86 (d, |
| | | | | J = 8.78 Hz, 2 H) 8.28 (s, 1 H) 8.99 (br. |
| | | | | s., 1 H) 9.63 (s, 1 H) |
| 111 | 3-({6-[(4-chlorophenyl)amino]-4- | 2.55^{a} | 461.1 (M + H)⁺ | ¹H NMR (400 MHz, DMSO-d₆) δ ppm |
| | pyrimidinyl}amino)-4- | | | 0.97 (t, J = 7.03 Hz, 6 H) 2.43 (d, |
| | (diethylamino)-N- | | | J = 5.02 Hz, 3 H) 3.11 (q, J = 7.03 Hz, 4 |
| | methylbenzenesulfonamide | | | H) 6.04 (s, 1 H) 7.29 (d, J = 8.78 Hz, 1 |
| | trifluoroacetate | | | H) 7.33-7.37 (m, 1 H) 7.39 (d, J = 8.78 Hz, |
| | | | | 2 H) 7.50-7.58 (m, 3 H) 7.92 (d, |
| | | | | J = 1.51 Hz, 1 H) 8.37 (s, 1 H) |
| | | | | 9.07-9.14 (m, 1 H) 9.77 (br s., 1 H) |
| 112 | 3-({6-[(4-chlorophenyl)amino]-4- | 2.50^{a} | 487.2 (M + H)⁺ | ¹H NMR (400 MHz, DMSO-d₆) δ ppm |
| | pyrimidinyl}amino)-4-(2,5- | | | 1.08 (d, J = 6.02 Hz, 6 H) |
| | dimethyl-1-pyrrolidinyl)-N- | | | 1.61-1.71 (m, 2 H) 1.95-2.04 (m, 2 H) 2.41 (d, |
| | methylbenzenesulfonamide | | | J = 4.02 Hz, 3 H) 3.64-3.75 (m, 2 H) |
| | trifluoroacetate | | | 6.04-6.10 (m, 1 H) 7.33-7.39 (m, 1 |
| | | | | H) 7.40-7.45 (m, 2 H) 7.55 (d, J = 8.53 Hz, |
| | | | | 3 H) 7.78-7.83 (m, 1 H) 8.46 (s, |
| | | | | 1 H) 9.62 (br. s., 1 H) 10.29 (br. s., 1 |
| | | | | H) |
| 113 | 3-({6-[(4-chlorophenyl)amino]-4- | 2.45^{a} | 473.1 (M + H)⁺ | ¹H NMR (400 MHz, DMSO-d₆) δ ppm |
| | pyrimidinyl}amino)-N-methyl-4- | | | 1.03 (d, J = 5.77 Hz, 3 H) |
| | (2-methyl-1- | | | 1.42-1.53 (m, 1 H) 1.61-1.74 (m, 1 H) |
| | pyrrolidinyl)benzenesulfonamide | | | 1.82-1.92 (m, 1 H) 2.05-2.15 (m, 1 H) |
| | trifluoroacetate | | | 2.40 (d, J = 4.77 Hz, 4 H) 3.17 (s, 1 H) |
| | | | | 3.48 (br. s., 1 H) 3.85-3.95 (m, 1 H) |
| | | | | 5.67-5.74 (m, 1 H) 7.00-7.05 (m, 2 |
| | | | | H) 7.20-7.26 (m, 1 H) 7.38 (d, J = 8.78 Hz, |
| | | | | 2 H) 7.49-7.57 (m, 3 H) 8.35 (s, |
| | | | | 1 H) 9.54 (br. s., 1 H) 9.94 (br. s., 1 H) |
| 114 | 3-({6-[(4-chlorophenyl)amino]-4- | 2.22^{a} | 404.0 (M + H)⁺ | ¹H NMR (400 MHz, DMSO-d₆) δ ppm |
| | pyrimidinyl}amino)-N,4- | | | 2.35 (s, 3 H) 2.49 (d, J = 5.02 Hz, 3 H) |
| | dimethylbenzenesulfonamide | | | 6.00 (s, 1 H) 7.41 (d, J = 8.78 Hz, 2 H) |
| | trifluoroacetate | | | 7.48 (q, J = 5.10 Hz, 1 H) 7.57 (s, 2 H) |
| | | | | 7.65 (d, J = 9.04 Hz, 2 H) 7.89 (s, 1 H) |
| | | | | 8.33 (s, 1 H) 9.04 (br. s., 1 H) |
| | | | | 9.53 (br. s., 1 H) |
| 115 | 3-(6-(4- | 1.07^{c} | 477.9 (M + H)⁺ | ¹H NMR (400 MHz, DMSO-d₆) δ ppm |
| | chlorophenylamino)pyrimidin-4- | | | 0.97 (d, J = 6.62 Hz, 6 H) 1.79 (m, |
| | ylamino)-4-(isobutylthio)-N- | | | J = 12.57, 6.28, 6.28 Hz, 1 H) 2.41 (d, |
| | methylbenzenesulfonamide | | | J = 5.07 Hz, 3 H) 2.86 (d, J = 6.62 Hz, 2 |
| | trifluoroacetate | | | H) 5.89 (s, 1 H) 7.29 (d, J = 9.04 Hz, 2 |
| | | | | H) 7.42-7.48 (m, 1 H) 7.54 (s, 2 H) |
| | | | | 7.57-7.62 (m, 2 H) 7.71 (s, 1 H) |
| | | | | 8.18 (s, 1 H) 8.76 (s, 1 H) 9.32 (s, 1 H) |
| 116 | 4-(isobutylthio)-N-methyl-3-(6- | 1.14^{c} | 511.9 (M + H)⁺ | ¹H NMR (400 MHz, DMSO-d₆) δ ppm |
| | (4- | | | 0.95 (d, J = 7.06 Hz, 6 H) |
| | (trifluoromethyl)phenylamino)pyrimidin- | | | 1.72-1.85 (m, 1 H) 2.40 (d, J = 5.29 Hz, 3 H) |
| | 4- | | | 2.85 (d, J = 7.06 Hz, 2 H) 5.93 (s, 1 H) |
| | ylamino)benzenesulfonamide | | | 7.44-7.47 (m, 1 H) 7.54 (s, 2 H) 7.58 (d, |
| | trifluoroacetate | | | J = 8.82 Hz, 2 H) 7.68 (s, 1 H) 7.79 (d, |
| | | | | J = 8.38 Hz, 2 H) 8.23 (s, 1 H) 8.91 (s, |
| | | | | 1 H) 9.62 (s, 1 H) |
| 117 | 4-(isobutylthio)-3-(6-(4- | 1.09^{c} | 486.0 (M + H)⁺ | ¹H NMR (400 MHz, DMSO-d₆) δ ppm |
| | isopropylphenylamino)pyrimidin- | | | 0.95 (d, J = 6.62 Hz, 6 H) 1.15 (d, |
| | 4-ylamino)-N- | | | J = 7.06 Hz, 6 H) 1.79 (m, J = 6.62 Hz, 1 |
| | methylbenzenesulfonamide | | | H) 2.38 (d, J = 4.85 Hz, 3 H) |
| | trifluoroacetate | | | 2.76-2.83 (m, 1 H) 2.85 (d, J = 6.62 Hz, 2 H) |
| | | | | 5.89 (s, 1 H) 7.14 (d, 2 H) 7.35 (d, |
| | | | | J = 8.38 Hz, 2 H) 7.45 (q, J = 5.15 Hz, 1 |
| | | | | H) 7.52 (s, 2 H) 7.70 (s, 1 H) 8.15 (s, |
| | | | | 1 H) 8.86 (br. s., 1 H) 9.25 (br. s., 1 H) |
| 118 | 3-{[6-({4- | 2.36^{a} | 520.1 (M + H)⁺ | ¹H NMR (400 MHz, METHANOL-d₄) δ |
| | [(difluoromethyl)oxy]phenyl}amino)- | | | ppm 2.56 (s, 3 H) 4.77 (q, J = 8.37 Hz, |
| | 4-pyrimidinyl]amino}-N- | | | 2 H) 6.06 (s, 1 H) 6.64-7.04 (m, 1 H) |
| | methyl-4-[(2,2,2- | | | 7.23 (d, J = 9.03 Hz, 2 H) 7.39 (d, |
| | trifluoroethyl)oxy]benzenesulfonamide | | | J = 8.78 Hz, 1 H) 7.43-7.48 (m, 2 H) |
| | trifluoroacetate | | | 7.78 (dd, J = 8.78, 2.26 Hz, 1 H) |
| | | | | 8.04 (d, J = 2.26 Hz, 1 H) 8.29 (s, 1 H) |
| 119 | N-methyl-4-[(2,2,2- | 2.44^{a} | 538.1 (M + H)⁺ | ¹H NMR (400 MHz, DMSO-d₆) δ ppm |
| | trifluoroethyl)oxy]-3-{[6-({4- | | | 2.43 (d, J = 5.02 Hz, 3 H) 4.91 (q, |
| | [(trifluoromethyl)oxy]phenyl}amino)- | | | J = 8.78 Hz, 2 H) 6.16 (s, 1 H) 7.32 (d, |
| | 4- | | | J = 8.78 Hz, 2 H) 7.41 (d, J = 8.03 Hz, 2 |
| | pyrimidinyl]amino}benzenesulfonamide | | | H) 7.54 (d, J = 8.03 Hz, 1 H) 7.67 (d, |
| | trifluoroacetate | | | J = 9.03 Hz, 2 H) 8.14 (br. s., 1 H) |
| | | | | 8.29 (s, 1 H) 8.89 (br. s., 1 H) 9.50 (br. s., 1 |
| | | | | H) |
| 120 | 3-({6-[(3,4- | 2.24^{a} | 490.0 (M + H)⁺ | ¹H NMR (400 MHz, DMSO-d₆) δ ppm |
| | difluorophenyl)amino]-4- | | | 2.44 (d, J = 5.02 Hz, 3 H) 4.93 (q, |
| | pyrimidinyl}amino)-N-methyl-4- | | | J = 8.95 Hz, 2 H) 6.16 (s, 1 H) |
| | [(2,2,2- | | | 7.24-7.30 (m, 1 H) 7.36-7.49 (m, 3 H) |
| | trifluoroethyl)oxy]benzenesulfonamide | | | 7.60 (dd, J = 8.53, 1.76 Hz, 1 H) |
| | hydrochloride | | | 7.79-7.87 (m, 1 H) 8.05 (br. s., 1 H) 8.34 (s, |
| | | | | 1 H) 9.26 (none, 1 H) 9.76-9.87 (m, |
| | | | | 1 H) |
| 121 | 3-({6-[(4-cyanophenyl)amino]-4- | 2.27^{a} | 479.0 (M + H)⁺ | ¹H NMR (400 MHz, DMSO-d₆) δ ppm |
| | pyrimidinyl}amino)-N-methyl-4- | | | 2.44 (d, J = 5.02 Hz, 3 H) 4.91 (q, |
| | [(2,2,2- | | | J = 8.78 Hz, 2 H) 6.21 (s, 1 H) |
| | trifluoroethyl)oxy]benzenesulfonamide | | | 7.37-7.45 (m, 2 H) 7.54 (dd, J = 8.78, 2.26 Hz, |
| | trifluoroacetate | | | 1 H) 7.72 (d, J = 8.78 Hz, 2 H) |
| | | | | 7.84 (d, J = 8.78 Hz, 2 H) 8.14 (d, J = 2.26 Hz, |
| | | | | 1 H) 8.33 (s, 1 H) 8.89 (s, 1 H) |
| | | | | 9.73 (s, 1 H) |
| 122 | 3-(6-(4- | 1.12^{c} | 450.0 (M + H)⁺ | ¹H NMR (400 MHz, DMSO-d₆) δ ppm |
| | chlorophenylamino)pyrimidin-4- | | | 1.23 (t, J = 7.28 Hz, 3 H) 2.41 (d, |
| | ylamino)-4-(ethylthio)-N- | | | J = 5.07 Hz, 3 H) 3.00 (q, J = 7.28 Hz, 2 |
| | methylbenzenesulfonamide | | | H) 5.87 (s, 1 H) 7.30 (d, J = 8.82 Hz, 2 |
| | trifluoroacetate | | | H) 7.47 (q, J = 5.07 Hz, 1 H) |
| | | | | 7.54-7.56 (m, 2 H) 7.59 (d, J = 8.82 Hz, 2 H) |
| | | | | 7.70 (d, J = 1.32 Hz, 1 H) 8.19 (s, 1 H) |
| | | | | 8.84 (s, 1 H) 9.37 (s, 1 H) |
| 123 | 4-(ethylthio)-N-methyl-3-(6-(4- | 1.21^{c} | 484.1 (M + H)⁺ | ¹H NMR (400 MHz, DMSO-d₆) δ ppm |
| | (trifluoromethyl)phenylamino)pyrimidin- | | | 1.24 (t, J = 7.39 Hz, 3 H) 2.41 (d, |
| | 4- | | | J = 5.07 Hz, 3 H) 3.01 (q, J = 7.50 Hz, 2 |
| | ylamino)benzenesulfonamide | | | H) 5.96 (s, 1 H) 7.49 (q, J = 5.29 Hz, 1 |
| | trifluoroacetate | | | H) 7.54-7.62 (m, 4 H) 7.70 (d, J = 1.54 Hz, |
| | | | | 1 H) 7.81 (d, J = 8.60 Hz, 2 H) |
| | | | | 8.26 (s, 1 H) 9.01 (s, 1 H) 9.75 (s, 1 H) |
| 124 | 4-(ethylthio)-3-(6-(4- | 1.15^{c} | 458.1 (M + H)⁺ | ¹H NMR (400 MHz, DMSO-d₆) δ ppm |
| | isopropylphenylamino)pyrimidin- | | | 1.16 (d, J = 6.84 Hz, 6 H) 1.23 (t, |
| | 4-ylamino)-N- | | | J = 7.17 Hz, 3 H) 2.40 (d, J = 5.07 Hz, 3 |
| | methylbenzenesulfonamide | | | H) 2.76-2.86 (m, 1 H) 2.99 (q, J = 7.28 Hz, |
| | trifluoroacetate | | | 2 H) 5.90 (s, 1 H) 7.13 (d, J = 8.38 Hz, |
| | | | | 2 H) 7.38 (d, J = 8.60 Hz, 2 H) |
| | | | | 7.43-7.49 (m, 1 H) 7.53 (s, 2 H) 7.73 (s, 1 |
| | | | | H) 8.13 (s, 1 H) 8.73 (br. s., 1 H) |
| | | | | 9.13 (br. s., 1 H) |
| 125 | 3-(6-(4- | 1.03^{c} | 503.8 (M + H)⁺ | ¹H NMR (400 MHz, DMSO-d₆) δ ppm |
| | chlorophenylamino)pyrimidin-4- | | | 2.41 (d, J = 4.85 Hz, 3 H) 4.09 (q, |
| | ylamino)-N-methyl-4-(2,2,2- | | | J = 10.14 Hz, 2 H) 5.91 (s, 1 H) 7.31 (d, |
| | trifluoroethylthio)benzenesulfonamide | | | J = 9.26 Hz, 2 H) 7.51-7.61 (m, 4 H) |
| | trifluoroacetate | | | 7.72 (d, J = 2.21 Hz, 1 H) 7.80 (d, |
| | | | | J = 8.38 Hz, 1 H) 8.21 (s, 1 H) 9.10 (s, |
| | | | | 1 H) 9.44 (s, 1 H) |
| 126 | N-methyl-4-(2,2,2- | 1.11^{c} | 538.0 (M + H)⁺ | ¹H NMR (400 MHz, DMSO-d₆) δ ppm |
| | trifluoroethylthio)-3-(6-(4- | | | 2.42 (d, J = 5.29 Hz, 3 H) 4.11 (q, |
| | (trifluoromethyl)phenylamino)pyrimidin- | | | J = 10.44 Hz, 2 H) 5.99 (s, 1 H) 7.54 (q, |
| | 4- | | | J = 4.85 Hz, 1 H) 7.57-7.64 (m, 3 H) |
| | ylamino)benzenesulfonamide | | | 7.73 (d, J = 1.76 Hz, 1 H) |
| | trifluoroacetate | | | 7.77-7.84 (m, 3 H) 8.27 (s, 1 H) 9.24 (s, 1 H) |
| | | | | 9.75 (s, 1 H) |
| 127 | 3-(6-(4- | 1.05^{c} | 512.0 (M + H)⁺ | ¹H NMR (400 MHz, DMSO-d₆) δ ppm |
| | isopropylphenylamino)pyrimidin- | | | 1.16 (d, J = 7.06 Hz, 6 H) 2.40 (d, |
| | 4-ylamino)-N-methyl-4-(2,2,2- | | | J = 5.29 Hz, 3 H) 2.84 (m, J = 13.84, |
| | trifluoroethylthio)benzenesulfonamide | | | 6.90, 6.90, 6.90, 6.90 Hz, 1 H) |
| | trifluoroacetate | | | 4.12 (q, J = 10.14 Hz, 2 H) 5.91 (s, 1 H) |
| | | | | 7.19 (d, 2 H) 7.34 (d, J = 8.38 Hz, 2 H) |
| | | | | 7.55 (q, J = 5.15 Hz, 1 H) 7.60 (dd, J = 8.38, |
| | | | | 1.76 Hz, 1 H) 7.73 (d, J = 2.21 Hz, 1 H) |
| | | | | 7.82 (d, J = 8.38 Hz, 1 H) 8.25 (s, 1 H) |
| | | | | 9.47 (br. s., 1 H) 9.65 (br. s., 1 H) |
| 128 | 4-fluoro-N-methyl-3-{[6-({4- | 1.76^{a} | 458.1 (M + H)⁺ | ¹H NMR (400 MHz, DMSO-d₆) δ ppm |
| | [(trifluoromethyl)oxy]phenyl}amino)- | | | 2.45 (d, J = 5.02 Hz, 3 H) 6.32 (s, 1 H) |
| | 4- | | | 7.32 (d, J = 8.53 Hz, 2 H) |
| | pyrimidinyl]amino}benzenesulfonamide | | | 7.45-7.54 (m, 3 H) 7.65-7.72 (m, 2 H) 8.33 (s, |
| | trifluoroacetate | | | 1 H) 8.52 (dd, J = 7.53, 1.76 Hz, 1 H) |
| | | | | 9.33 (s, 1 H) 9.52 (s, 1 H) |
| 129 | 3-{[6-({4- | 2.13^{a} | 440.0 (M + H)⁺ | ¹H NMR (400 MHz, DMSO-d₆) δ ppm |
| | [(difluoromethyl)oxy]phenyl}amino)- | | | 2.51 (d, J = 5.02 Hz, 3 H) 6.35 (s, 1 H) |
| | 4-pyrimidinyl]amino}-4-fluoro- | | | 7.18-7.23 (m, 1 H) 7.50-7.58 (m, 3 |
| | N-methylbenzenesulfonamide | | | H) 7.65 (d, J = 9.03 Hz, 2 H) 8.36 (s, 1 |
| | trifluoroacetate | | | H) 8.60 (dd, J = 7.53, 2.01 Hz, 1 H) |
| | | | | 9.32 (s, 1 H) 9.41 (s, 1 H) |
| 130 | 4-chloro-N-methyl-3-[(6-{[4- | 1.88^{a} | 458.1 (M + H)⁺ | ¹H NMR (400 MHz, DMSO-d₆) δ ppm |
| | (trifluoromethyl)phenyl]amino}-4- | | | 2.47 (d, J = 5.02 Hz, 3 H) 6.30 (s, 1 H) |
| | pyrimidinyl)amino]benzenesulfonamide | | | 7.53 (dd, J = 8.41, 2.13 Hz, 1 H) |
| | trifluoroacetate | | | 7.59 (q, J = 4.77 Hz, 1 H) 7.66 (d, J = 8.78 Hz, |
| | | | | 2 H) 7.77 (d, J = 8.28 Hz, 1 H) |
| | | | | 7.83 (d, J = 8.53 Hz, 2 H) 8.20 (d, J = 2.26 Hz, |
| | | | | 1 H) 8.36 (s, 1 H) 9.28 (s, 1 H) |
| | | | | 9.80 (s, 1 H) |
| 131 | 3-({6-[(4-cyanophenyl)amino]-4- | 2.30^{a} | 459.1 (M + H)⁺ | ¹H NMR (400 MHz, DMSO-d₆) δ ppm |
| | pyrimidinyl}amino)-N-methyl-4- | | | 2.47 (d, 3H, obscured by solvent) |
| | (methylsulfonyl)benzenesulfonamide | | | 3.31 (s, 3H) 6.37-6.41 (m, 1 H) 7.70 (dd, |
| | trifluoroacetate | | | J = 8.28, 1.76 Hz, 1 H) 7.75 (d, J = 9.03 Hz, |
| | | | | 3 H) 7.80 (q, J = 4.94 Hz, 1 H) |
| | | | | 7.84-7.88 (m, 3 H) 8.13 (d, J = 8.28 Hz, 1 |
| | | | | H) 8.41-8.44 (m, 2 H) 9.05 (s, 1 H) |
| | | | | 9.91 (s, 1 H) |
| 132 | 3-({6-[(3,4- | 1.69^{a} | 470.1 (M + H)⁺ | ¹H NMR (400 MHz, DMSO-d₆) δ ppm |
| | difluorophenyl)amino]-4- | | | 2.47 (d, 3H, obscured by solvent) |
| | pyrimidinyl}amino)-N-methyl-4- | | | 3.31 (s, 3 H) 6.29 (s, 1 H) 7.24-7.32 (m, 1 |
| | (methylsulfonyl)benzenesulfonamide | | | H) 7.39 (m, J = 10.54 Hz, 1 H) |
| | trifluoroacetate | | | 7.68 (dd, J = 8.28, 1.76 Hz, 1 H) 7.80 (d, |
| | | | | J = 5.02 Hz, 1 H) 7.83-7.91 (m, 1 H) |
| | | | | 8.12 (d, J = 8.28 Hz, 1 H) 8.36 (s, 1 H) |
| | | | | 8.42 (d, J = 1.51 Hz, 1 H) 8.98 (s, 1 H) |
| | | | | 9.62 (s, 1 H) |
| 133 | 3-(6-(1H-indazol-5- | 0.74^{c} | 474.2 (M + H)⁺ | ¹H NMR (400 MHz, DMSO-d₆) δ ppm |
| | ylamino)pyrimidin-4-ylamino)-N- | | | 2.44 (d, J = 4.85 Hz, 3 H) 3.26 (s, 3 H) |
| | methyl-4- | | | 6.19 (s, 1 H) 7.32 (dd, J = 8.93, 1.87 Hz, |
| | (methylsulfonyl)benzenesulfonamide | | | 1 H) 7.54 (d, J = 8.82 Hz, 1 H) |
| | | | | 7.71 (dd, J = 8.49, 1.43 Hz, 1 H) 7.77 (q, 1 |
| | | | | H) 7.86 (s, 1 H) 8.05 (s, 1 H) 8.10 (d, |
| | | | | J = 8.38 Hz, 1 H) 8.25 (s, 1 H) 8.32 (s, |
| | | | | 1 H) 9.23 (br. s., 1 H) 9.76 (br. s., 1 H) |
| 134 | 3-(6-(4- | 0.87^{c} | 473.1 (M + H)⁺ | ¹H NMR (400 MHz, DMSO-d₆) δ ppm |
| | (cyanomethyl)phenylamino)pyrimidin- | | | 3.29 (s, 3 H) 3.96 (s, 2 H) 6.31 (s, 1 |
| | 4-ylamino)-N-methyl-4- | | | H) 7.28 (d, 2 H) 7.57 (d, J = 8.60 Hz, 2 |
| | (methylsulfonyl)benzenesulfonamide | | | H) 7.65 (dd, J = 8.38, 1.54 Hz, 1 H) |
| | | | | 7.79 (q, J = 4.78 Hz, 1 H) 8.09 (d, |
| | | | | J = 8.38 Hz, 1 H) 8.32 (s, 1 H) 8.41 (d, |
| | | | | J = 1.54 Hz, 1 H) 8.94 (br. s., 1 H) |
| | | | | 9.54 (s, 1 H) |
| 135 | 4-(tert-butylsulfonyl)-3-(6-(4- | 1.16^{c} | 509.9 (M + H)⁺ | ¹H NMR (400 MHz, DMSO-d₆) δ ppm |
| | chlorophenylamino)pyrimidin-4- | | | 1.22 (s, 9 H) 6.32 (s, 1 H) 7.33 (d, 2 |
| | ylamino)-N- | | | H) 7.54 (dd, J = 8.49, 1.65 Hz, 1 H) |
| | methylbenzenesulfonamide | | | 7.62 (d, J = 8.82 Hz, 2 H) 7.79 (q, |
| | trifluoroacetate | | | J = 4.78 Hz, 1 H) 7.96 (d, J = 8.38 Hz, 1 |
| | | | | H) 8.35 (s, 1 H) 8.63 (d, J = 1.54 Hz, 1 |
| | | | | H) 9.17 (s, 1 H) 9.59 (s, 1 H) |
| 136 | 3-({6-[(4-chlorophenyl)amino]-4- | 2.43^{a} | 515.9 (M + H)⁺ | ¹H NMR (400 MHz, DMSO-d₆) δ ppm |
| | pyrimidinyl}amino)-N-methyl-4- | | | 1.41 (s, 6 H) 2.45 (s, 3 H) 6.10 (s, 1 |
| | [(2,2,2-trifluoro-1,1- | | | H) 7.32 (s, 2 H) 7.39 (d, J = 8.53 Hz, 1 |
| | dimethylethyl)oxy]benzenesulfonamide | | | H) 7.49 (dd, J = 8.53, 2.26 Hz, 2 H) |
| | | | | 7.65 (d, J = 9.03 Hz, 2 H) |
| | | | | 8.16-8.20 (m, 1 H) 8.26-8.30 (m, 1 H) 8.67 (s, |
| | | | | 1 H) 9.39 (s, 1 H) |
| 137 | 3-({6-[(3-bromophenyl)amino]-4- | 1.59^{c} | 436.0 (M + H)⁺ | ¹H NMR (400 MHz, DMSO-d6) δ ppm |
| | pyrimidinyl}amino)-N- | | | 9.67 (br. s., 1H), 9.54 (br. s., 1H), |
| | methylbenzenesulfonamide | | | 8.39 (s, 1H), 8.11 (br. s., 1H), 8.03 (br. s., |
| | | | | 1H), 7.93 (d, J = 7.53 Hz, 1H), |
| | | | | 7.41-7.58 (m, 3H), 7.34 (d, J = 7.53 Hz, |
| | | | | 1H), 7.25 (t, J = 7.91 Hz, 1H), 7.13 (d, |
| | | | | J = 7.28 Hz, 1H), 6.25 (s, 1H), 2.45 (d, |
| | | | | J = 4.52 Hz, 3H) |
| 138 | 3-({6-[(3-bromo-4- | 1.67^{c} | 469.8 (M + H)⁺ | ¹H NMR (400 MHz, DMSO-d6) δ |
| | chlorophenyl)amino]-4- | | | 9.65 (s, 1H), 9.57 (s, 1H), 8.42 (s, 1H), |
| | pyrimidinyl}amino)-N- | | | 8.23 (d, J = 2.26 Hz, 1H), 8.09 (s, 1H), |
| | methylbenzenesulfonamide | | | 7.93 (d, J = 8.03 Hz, 1H), 7.59 (dd, J = 2.26, |
| | | | | 8.78 Hz, 1H), 7.50-7.56 (m, |
| | | | | 2H), 7.45 (q, J = 4.85 Hz, 1H), |
| | | | | 7.35 (d, J = 7.78 Hz, 1H), 6.20 (s, 1H), |
| | | | | 2.45 (d, J = 4.77 Hz, 3H) |
| 139 | 3-[(6-{[3,4- | 0.83^{c} | 462.0 (M + H)⁺ | ¹H NMR (400 MHz, METHANOL-d₄) δ |
| | bis(methyloxy)phenyl]amino}-4- | | | ppm 2.50 (s, 3 H) 2.53 (s, 3 H) |
| | pyrimidinyl)amino]-N-methyl-4- | | | 3.80 (s, 3 H) 3.83 (s, 3 H) 5.70 (s, 1 H) |
| | (methylthio)benzenesulfonamide | | | 6.87 (dd, J = 8.38, 2.43 Hz, 1 H) |
| | | | | 6.92 (d, J = 2.43 Hz, 1 H) 6.99 (d, J = 8.60 Hz, |
| | | | | 1 H) 7.55 (d, J = 8.60 Hz, 1 H) |
| | | | | 7.72 (d, J = 1.98 Hz, 1 H) 7.80 (dd, J = 8.38, |
| | | | | 1.98 Hz, 1 H) 8.22 (s, 1 H) |
| 140 | N-methyl-4-methylsulfanyl-3-[6- | 0.86^{c} | 492.0 (M + H)⁺ | ¹H NMR (400 MHz, METHANOL-d₄) δ |
| | (3,4,5-trimethoxy-phenylamino)- | | | ppm 2.51 (s, 3 H) 2.53 (s, 3 H) |
| | pyrimidin-4-ylamino]- | | | 3.74 (s, 3 H) 3.80 (s, 6 H) 5.77 (s, 1 H) |
| | benzenesulfonamide | | | 6.65 (s, 2 H) 7.56 (d, J = 8.38 Hz, 1 H) |
| | trifluoroacetate | | | 7.73 (d, J = 1.98 Hz, 1 H) 7.82 (dd, |
| | | | | J = 8.38, 1.98 Hz, 1 H) 8.25 (d, 1 H) |
| 141 | 3-[6-(3,5-dimethoxy- | 0.90^{c} | 462.0 (M + H)⁺ | ¹H NMR (400 MHz, DMSO-d₆) δ ppm |
| | phenylamino)-pyrimidin-4- | | | 2.38 (d, J = 5.29 Hz, 3 H) 3.68 (s, 9 H) |
| | ylamino]-N-methyl-4- | | | 5.83-5.89 (m, 1 H) 6.18-6.24 (m, 1 |
| | methylsulfanyl- | | | H) 6.66 (m, J = 1.76 Hz, 2 H) 7.46 (m, |
| | benzenesulfonamide | | | J = 14.55 Hz, 1 H) 7.50 (d, J = 8.38 Hz, |
| | trifluoroacetate | | | 1 H) 7.64 (m, J = 2.65 Hz, 2 H) 8.26 (s, |
| | | | | 1 H) 9.44 (br. s., 1 H) 9.67 (br. s., 1 H) |
| 142 | 3-[6-(4-cyano-phenylamino)- | 0.92^{c} | 426.9 (M + H)⁺ | ¹H NMR (400 MHz, DMSO-d₆) δ ppm |
| | pyrimidin-4-ylamino]-N-methyl-4- | | | 2.47 (s, 3H and d, 3H, obscured by |
| | methylsulfanyl- | | | solvent) 5.90 (br. s., 1 H) |
| | benzenesulfonamide | | | 7.40-7.54 (m, 2 H) 7.61-7.80 (m, 6 H) 8.30 (s, |
| | trifluoroacetate | | | 1 H) 9.25 (br. s., 1 H) 9.92 (br. s., 1 H) |
| 143 | 3-[6-(benzo[1,3]dioxol-5- | 0.65^{c} | 446.0 (M + H)⁺ | ¹H NMR (400 MHz, DMSO-d₆) δ ppm |
| | ylamino)-pyrimidin-4-ylamino]-N- | | | 2.38 (d, J = 4.85 Hz, 3 H) 2.47 (s, 3H, |
| | methyl-4-methylsulfanyl- | | | obscured by solvent) 5.74 (s, 1 H) |
| | benzenesulfonamide | | | 5.99 (s, 2 H) 6.78 (dd, J = 8.60, 1.98 Hz, |
| | trifluoroacetate | | | 1 H) 6.88 (d, J = 8.38 Hz, 1 H) |
| | | | | 7.08 (s, 1 H) 7.45-7.53 (m, 2 H) |
| | | | | 7.60-7.67 (m, 2 H) 8.25 (s, 1 H) 9.57 (br. s., |
| | | | | 1 H) 9.75 (br. s., 1 H) |
| 144 | 3-[6-(benzothiazol-6-ylamino)- | 0.84^{c} | 458.8 (M + H)⁺ | ¹H NMR (400 MHz, DMSO-d₆) δ ppm |
| | pyrimidin-4-ylamino]-N-methyl-4- | | | 2.40 (d, J = 5.07 Hz, 3 H) 5.89 (s, 1 H) |
| | methylsulfanyl- | | | 7.45-7.57 (m, 3 H) 7.62-7.70 (m, 2 |
| | benzenesulfonamide | | | H) 8.02 (d, J = 8.82 Hz, 1 H) 8.33 (s, 1 |
| | trifluoroacetate | | | H) 8.41 (d, J = 1.98 Hz, 1 H) 9.27 (s, 1 |
| | | | | H) 9.49 (br. s., 1 H) 10.00 (br. s., 1 H) |
| 145 | N-methyl-3-[6-(2-methyl- | 0.89^{c} | 472.9 (M + H)⁺ | ¹H NMR (400 MHz, DMSO-d₆) δ ppm |
| | benzothiazol-5-ylamino)- | | | 2.38 (d, J = 5.29 Hz, 3 H) 2.47 (s, 3H, |
| | pyrimidin-4-ylamino]-4- | | | obscured by solvent) 2.75 (s, 3 H) |
| | methylsulfanyl- | | | 5.88 (s, 1 H) 7.39-7.53 (m, 3 H) |
| | benzenesulfonamide | | | 7.61-7.68 (m, 2 H) 7.94 (d, J = 8.82 Hz, 1 |
| | trifluoroacetate | | | H) 8.12 (s, 1 H) 8.27-8.31 (m, 1 H) |
| | | | | 9.44 (br. s., 1 H) 9.89 (br. s., 1 H) |
| 146 | 3-[6-(3-chloro-4-hydroxy- | 0.81^{c} | 451.9 (M + H)⁺ | ¹H NMR (400 MHz, DMSO-d₆) δ ppm |
| | phenylamino)-pyrimidin-4- | | | 2.38 (d, J = 4.85 Hz, 3 H) 2.47 (s, 3H, |
| | ylamino]-N-methyl-4- | | | obscured by solvent) 5.70 (s, 1 H) |
| | methylsulfanyl- | | | 6.92 (d, J = 8.38 Hz, 1 H) 7.11 (dd, |
| | benzenesulfonamide | | | J = 8.60, 2.43 Hz, 1 H) 7.42-7.54 (m, |
| | trifluoroacetate | | | 3 H) 7.57-7.68 (m, 2 H) 8.25 (s, 1 H) |
| | | | | 9.41-9.52 (m, 1 H) 9.64 (br. s., 1 H) |
| | | | | 10.15 (br. s., 1 H) |
| 147 | 3-[6-(3,4-difluoro-phenylamino)- | 0.93^{c} | 437.9 (M + H)⁺ | ¹H NMR (400 MHz, DMSO-d₆) δ ppm |
| | pyrimidin-4-ylamino]-N-methyl-4- | | | 2.39 (d, J = 4.85 Hz, 3 H) 2.47 (s, 3H, |
| | methylsulfanyl- | | | obscured by solvent) 5.79 (s, 1 H) |
| | benzenesulfonamide | | | 7.16-7.22 (m, 1 H) 7.35 (m, J = 10.58 Hz, |
| | trifluoroacetate | | | 1 H) 7.44-7.52 (m, 2 H) |
| | | | | 7.62-7.66 (m, 2 H) 7.77 (ddd, J = 13.12, |
| | | | | 7.61, 2.65 Hz, 1 H) 8.28 (s, 1 H) |
| | | | | 9.34 (br. s., 1 H) 9.75 (s, 1 H) |
| 148 | N-methyl-4-methylsulfanyl-3-[6- | 0.84^{c} | 486.9 (M + H)⁺ | ¹H NMR (400 MHz, DMSO-d₆) δ ppm |
| | (4-morpholin-4-yl-phenylamino)- | | | 2.38 (d, J = 4.85 Hz, 3 H) 2.47 (s, 3H, |
| | pyrimidin-4-ylamino]- | | | obscured by solvent) 3.02-3.11 (m, 4 |
| | benzenesulfonamide | | | H) 3.65-3.75 (m, 4 H) 5.71 (br. s., 1 |
| | trifluoroacetate | | | H) 6.96 (d, J = 9.26 Hz, 2 H) 7.21 (d, |
| | | | | J = 8.82 Hz, 2 H) 7.48 (m, J = 5.29 Hz, 1 |
| | | | | H) 7.52 (d, J = 8.38 Hz, 1 H) 7.61 (d, |
| | | | | J = 1.76 Hz, 1 H) 7.67 (dd, 1 H) 8.28 (s, |
| | | | | 1 H) 9.79 (br. s., 1 H) 9.92 (br. s., 1 H) |
| 149 | 3-[6-(2,3-dihydro- | 0.85^{c} | 460.1 (M + H)⁺ | ¹H NMR (400 MHz, DMSO-d₆) δ ppm |
| | benzo[1,4]dioxin-6-ylamino)- | | | 2.40 (d, J = 4.85 Hz, 3 H) 2.47 (s, 3H, |
| | pyrimidin-4-ylamino]-N-methyl-4- | | | obscured by solvent) 4.18-4.24 (m, 4 |
| | methylsulfanyl- | | | H) 5.76 (s, 1 H) 6.77-6.86 (m, 2 H) |
| | benzenesulfonamide | | | 7.00 (d, J = 0.88 Hz, 1 H) 7.48 (q, |
| | trifluoroacetate | | | J = 5.00 Hz, 1 H) 7.52 (d, J = 8.60 Hz, 1 |
| | | | | H) 7.63 (d, J = 1.98 Hz, 1 H) 7.66 (dd, |
| | | | | J = 8.16, 1.98 Hz, 1 H) 8.26 (s, 1 H) |
| | | | | 9.56 (br. s., 1 H) 9.68 (br. s., 1 H) |
| 150 | N-methyl-4-methylsulfanyl-3-[6- | 0.74^{c} | 485.0 (M + H)⁺ | ¹H NMR (400 MHz, DMSO-d₆) δ ppm |
| | (4-piperidin-1-yl-phenylamino)- | | | 1.60 (br. s., 2 H) 1.79 (br. s., 4 H) |
| | pyrimidin-4-ylamino]- | | | 2.40 (d, J = 4.85 Hz, 3 H) 2.47 (s, 3H, |
| | benzenesulfonamide | | | obscured by solvent) 3.38 (br. s., 4 H) |
| | trifluoroacetate | | | 5.80 (s, 1 H) 7.37-7.45 (m, 2 H) |
| | | | | 7.46-7.53 (m, 2 H) 7.53-7.59 (m, 2 H) |
| | | | | 7.62-7.67 (m, 2 H) 8.27 (s, 1 H) |
| | | | | 9.36 (br. s., 1 H) 9.77 (br. s., 1 H) |
| 151 | 3-[6-(3-ethynyl-phenylamino)- | 0.91^{c} | 426.0 (M + H)⁺ | ¹H NMR (400 MHz, DMSO-d₆) δ ppm |
| | pyrimidin-4-ylamino]-N-methyl-4- | | | 2.39 (d, J = 5.29 Hz, 3 H) 2.47 (s, 3H, |
| | methylsulfanyl- | | | obscured by solvent) 4.15 (s, 1 H) |
| | benzenesulfonamide | | | 5.86 (s, 1 H) 7.05 (d, 1 H) 7.26 (t, |
| | trifluoroacetate | | | J = 7.94 Hz, 1 H) 7.45-7.52 (m, 3 H) |
| | | | | 7.59-7.64 (m, 2 H) 7.76 (s, 1 H) |
| | | | | 8.24 (s, 1 H) 9.10 (br. s., 1 H) 9.58 (br. s., 1 |
| | | | | H) |
| 152 | 3-[6-(3,5-dichloro-4-hydroxy- | 0.86^{c} | 486.0 (M + H)⁺ | ¹H NMR (400 MHz, DMSO-d₆) δ ppm |
| | phenylamino)-pyrimidin-4- | | | 2.48 (d, J = 4.77 Hz, 3 H) 2.55 (s, 3H, |
| | ylamino]-N-methyl-4- | | | obscured by solvent) 5.82 (s, 1 H) |
| | methylsulfanyl- | | | 7.53 (q, J = 4.85 Hz, 1 H) |
| | benzenesulfonamide | | | 7.57-7.63 (m, 3 H) 7.70-7.75 (m, 2 H) 8.36 (s, |
| | trifluoroacetate | | | 1 H) 9.44 (br. s., 1 H) 9.68 (br. s., 1 H) |
| | | | | 9.97 (br. s., 1 H) |
| 153 | N-methyl-4-methylsulfanyl-3-{6- | 0.93^{c} | 498.9 (M + H)⁺ | ¹H NMR (400 MHz, DMSO-d₆) δ ppm |
| | [3-(2-methyl-thiazol-4-yl)- | | | 2.41 (d, J = 4.77 Hz, 3 H) 2.49 (s, 3H, |
| | phenylamino]-pyrimidin-4- | | | obscured by solvent) 2.71 (s, 3 H) |
| | ylamino}-benzenesulfonamide | | | 5.90 (s, 1 H) 7.38-7.43 (m, 1 H) |
| | trifluoroacetate | | | 7.45-7.57 (m, 3 H) 7.63-7.72 (m, 3 H) |
| | | | | 7.89 (s, 1 H) 7.97 (s, 1 H) 8.35 (s, 1 |
| | | | | H) 9.63 (br. s., 1 H) 9.99 (br. s., 1 H) |
| 154 | 3-(6-(3-methoxy-5- | 1.02^{c} | 499.9 (M + H)⁺ | ¹H NMR (400 MHz, DMSO-d₆) δ ppm |
| | (trifluoromethyl)phenylamino)pyrimidin- | | | 2.40 (d, J = 5.29 Hz, 3 H) 2.47 (s, 3H, |
| | 4-ylamino)-N-methyl-4- | | | obscured by solvent) 3.78 (s, 3 H) |
| | (methylthio)benzenesulfonamide | | | 5.84 (s, 1 H) 6.82 (s, 1 H) |
| | trifluoroacetate | | | 7.44-7.53 (m, 3 H) 7.57 (s, 1 H) 7.62-7.66 (m, |
| | | | | 2 H) 8.28 (s, 1 H) 9.17 (br. s., 1 H) |
| | | | | 9.70 (br. s., 1 H) |
| 155 | 3-[6-(1H-indol-5-ylamino)- | 0.83^{c} | 440.8 (M + H)⁺ | ¹H NMR (400 MHz, DMSO-d₆) δ ppm |
| | pyrimidin-4-ylamino]-N-methyl-4- | | | 2.35 (d, J = 4.85 Hz, 3 H) 2.47 (s, 3H, |
| | methylsulfanyl- | | | obscured by solvent) 5.75 (br. s., 1 H) |
| | benzenesulfonamide | | | 6.42 (br. s., 1 H) 7.00 (dd, J = 8.60, |
| | trifluoroacetate | | | 1.98 Hz, 1 H) 7.36-7.42 (m, 2 H) |
| | | | | 7.45 (q, J = 4.85 Hz, 1 H) |
| | | | | 7.48-7.53 (m, 2 H) 7.61-7.67 (m, 2 H) 8.25 (s, |
| | | | | 1 H) 9.61 (br. s., 1 H) 9.87 (br. s., 1 H) |
| | | | | 11.20 (br. s., 1 H) |
| 156 | N-methyl-4-methylsulfanyl-3-[6- | 0.74^{c} | 453.0 (M + H)⁺ | ¹H NMR (400 MHz, DMSO-d₆) δ ppm |
| | (quinolin-6-ylamino)-pyrimidin-4- | | | 2.41 (d, J = 4.85 Hz, 3 H) 2.47 (s, 3H, |
| | ylamino]-benzenesulfonamide | | | obscured by solvent) 5.96 (s, 1 H) |
| | trifluoroacetate | | | 7.47 (q, 1 H) 7.52 (d, J = 8.38 Hz, 1 H) |
| | | | | 7.62-7.68 (m, 2 H) 7.63 (s, 1 H) |
| | | | | 7.78 (dd, J = 8.38, 4.85 Hz, 1 H) 8.01 (m, |
| | | | | J = 2.21 Hz, 1 H) 8.10 (d, J = 9.26 Hz, 1 |
| | | | | H) 8.35 (s, 1 H) 8.56 (d, J = 1.76 Hz, 1 |
| | | | | H) 8.74 (d, J = 7.94 Hz, 1 H) 8.95 (dd, |
| | | | | J = 4.85, 1.32 Hz, 1 H) 9.19 (br. s., 1 H) |
| | | | | 10.01 (s, 1 H) |
| 157 | 3-[6-(3-chloro-4-cyano- | 1.03^{c} | 461.0 (M + H)⁺ | ¹H NMR (400 MHz, METHANOL-d₄) δ |
| | phenylamino)-pyrimidin-4- | | | ppm 2.55 (s, 3 H) 2.56 (s, 3 H) |
| | ylamino]-N-methyl-4- | | | 6.04 (s, 1 H) 7.56-7.61 (m, 2 H) |
| | methylsulfanyl- | | | 7.58-7.59 (m, 1 H) 7.80-7.84 (m, 2 H) |
| | benzenesulfonamide | | | 8.07 (d, J = 1.98 Hz, 1 H) 8.40 (s, 1 H) |
| | trifluoroacetate | | | |
| 158 | N-methyl-4-methylsulfanyl-3-[6- | 0.77^{c} | 482.9 (M + H)⁺ | ¹H NMR (400 MHz, DMSO-d₆) δ ppm |
| | (4-[1,2,4]triazol-4-ylmethyl- | | | 2.39 (d, J = 4.85 Hz, 3 H) 2.47 (s, 3H, |
| | phenylamino)-pyrimidin-4- | | | obscured by solvent) 5.34 (s, 2 H) |
| | ylamino]-benzenesulfonamide | | | 5.96 (s, 1 H) 7.24 (d, J = 8.38 Hz, 2 H) |
| | trifluoroacetate | | | 7.45-7.52 (m, 3 H) 7.55 (q, J = 4.85 Hz, |
| | | | | 1 H) 7.61-7.67 (m, 2 H) 7.95 (s, |
| | | | | 1 H) 8.27 (s, 1 H) 8.65 (s, 1 H) |
| | | | | 9.49 (br. s., 1 H) 9.98 (br. s., 1 H) |
| 159 | 3-[6-(1H-indazol-5-ylamino)- | 0.73^{c} | 442.0 (M + H)⁺ | ¹H NMR (400 MHz, DMSO-d₆) δ ppm |
| | pyrimidin-4-ylamino]-N-methyl-4- | | | 2.37 (d, J = 5.29 Hz, 3 H) 2.47 (s, 3H, |
| | methylsulfanyl- | | | obscured by solvent) 5.90 (s, 1 H) |
| | benzenesulfonamide | | | 7.33 (dd, 1 H) 7.45-7.51 (m, 2 H) |
| | trifluoroacetate | | | 7.55 (m, J = 5.29 Hz, 1 H) 7.60 (d, |
| | | | | J = 8.38 Hz, 1 H) 7.65 (d, J = 2.21 Hz, 1 |
| | | | | H) 7.90 (s, 1 H) 7.99 (s, 1 H) 8.22 (s, |
| | | | | 1 H) 9.15 (br. s., 1 H) 9.67 (br. s., 1 H) |
| 160 | 3-[6-(1H-indol-6-ylamino)- | 0.87^{c} | 441.0 (M + H)⁺ | ¹H NMR (400 MHz, METHANOL-d₄) δ |
| | pyrimidin-4-ylamino]-N-methyl-4- | | | ppm 2.39 (br. s., 3 H) 2.53 (s, 3 H) |
| | methylsulfanyl- | | | 5.76 (s, 1 H) 6.47 (dd, J = 3.09, 0.88 Hz, |
| | benzenesulfonamide | | | 2 H) 6.92 (dd, J = 8.38, 1.98 Hz, 1 |
| | trifluoroacetate | | | H) 7.30 (d, J = 3.31 Hz, 1 H) 7.36 (s, 1 |
| | | | | H) 7.52 (d, J = 8.38 Hz, 2 H) 7.59 (d, |
| | | | | J = 7.94 Hz, 1 H) 7.71 (d, J = 1.98 Hz, 1 |
| | | | | H) 7.76 (dd, J = 8.38, 1.98 Hz, 1 H) |
| | | | | 8.18-8.21 (m, 1 H) |
| 161 | N-methyl-4-(methylthio)-3-(6-(4- | 0.88^{c} | 486.0 (M + H)⁺ | ¹H NMR (400 MHz, DMSO-d₆) δ ppm |
| | (piperazin-1- | | | 2.39 (d, J = 5.07 Hz, 3 H) 2.47 (s, 3H, |
| | yl)phenylamino)pyrimidin-4- | | | obscured by solvent) 3.22 (br. s., 4 H) |
| | ylamino)benzenesulfonamide | | | 3.25-3.29 (m, 4 H) 5.73 (s, 1 H) |
| | trifluoroacetate | | | 6.97 (d, J = 9.04 Hz, 2 H) 7.30 (d, J = 9.04 Hz, |
| | | | | 2 H) 7.44-7.48 (m, 1 H) 7.50 (d, |
| | | | | J = 9.04 Hz, 1 H) 7.61-7.66 (m, 2 H) |
| | | | | 8.22 (s, 1 H) 8.77 (br. s., 2 H) |
| | | | | 9.33 (br. s., 1 H) 9.56 (br. s., 1 H) |
| 162 | N-methyl-3-(6-(4-methyl-2-oxo- | 0.86^{c} | 483.0 (M + H)⁺ | ¹H NMR (400 MHz, DMSO-d₆) δ ppm |
| | 1,2-dihydroquinolin-7- | | | 2.35 (d, J = 1.10 Hz, 3 H) 2.41 (d, |
| | ylamino)pyrimidin-4-ylamino)-4- | | | J = 4.85 Hz, 3 H) 2.52 (s, 3 H) 5.92 (s, |
| | (methylthio)benzenesulfonamide | | | 1 H) 6.20 (s, 1 H) 7.36 (dd, J = 8.93, |
| | trifluoroacetate | | | 2.09 Hz, 1 H) 7.47 (m, J = 5.29 Hz, 1 |
| | | | | H) 7.51 (d, J = 8.16 Hz, 1 H) 7.59 (d, 1 |
| | | | | H) 7.61-7.67 (m, 3 H) 8.28 (s, 1 H) |
| | | | | 9.18 (br. s., 1 H) 9.77 (br. s., 1 H) |
| | | | | 11.51 (br. s., 1 H) |
| 163 | 3-(6-(1-acetylindolin-6- | 0.84^{c} | 484.9 (M + H)⁺ | ¹H NMR (400 MHz, DMSO-d₆) δ ppm |
| | ylamino)pyrimidin-4-ylamino)-N- | | | 2.13 (s, 3 H) 2.39 (d, J = 4.85 Hz, 3 H) |
| | methyl-4- | | | 2.47 (s, 3H, obscured by solvent) |
| | (methylthio)benzenesulfonamide | | | 3.08 (t, J = 8.38 Hz, 2 H) 4.08 (t, |
| | trifluoroacetate | | | J = 8.60 Hz, 2 H) 5.80 (s, 1 H) 7.18 (s, |
| | | | | 2 H) 7.48 (q, J = 4.85 Hz, 1 H) 7.52 (d, |
| | | | | J = 8.38 Hz, 1 H) 7.63 (d, 1 H) |
| | | | | 7.67 (dd, J = 8.38, 1.76 Hz, 1 H) 8.01 (s, 1 |
| | | | | H) 8.30 (s, 1 H) 9.70 (br. s., 1 H) |
| | | | | 9.96 (br. s., 1 H) |
| 164 | N-methyl-3-[6-(2-methyl-4-oxo- | 0.91^{c} | 483.9 (M + H)⁺ | ¹H NMR (400 MHz, DMSO-d₆) δ ppm |
| | 4H-chromen-7-ylamino)- | | | 2.33 (s, 3 H) 2.40 (d, J = 4.85 Hz, 3 H) |
| | pyrimidin-4-ylamino]-4- | | | 2.47 (s, 3H, obscured by solvent) |
| | methylsulfanyl- | | | 5.91 (s, 1 H) 6.10 (s, 1 H) 7.34 (dd, J = 8.82, |
| | benzenesulfonamide | | | 1.76 Hz, 1 H) 7.44 (q, J = 4.85 Hz, 1 H) |
| | trifluoroacetate | | | 7.50 (d, J = 8.82 Hz, 1 H) |
| | | | | 7.60-7.66 (m, 2 H) 7.83 (d, J = 8.82 Hz, 1 H) |
| | | | | 8.19 (d, J = 1.32 Hz, 1 H) 8.33 (s, 1 H) |
| | | | | 9.11 (s, 1 H) 9.86 (s, 1 H) |
| 165 | 3-[6-(4-cyanomethyl- | 0.82^{c} | 441.0 (M + H)⁺ | ¹H NMR (400 MHz, DMSO-d₆) δ ppm |
| | phenylamino)-pyrimidin-4- | | | 2.39 (d, J = 4.85 Hz, 3 H) 2.47 (s, 3H, |
| | ylamino]-N-methyl-4- | | | obscured by solvent) 3.95 (s, 2 H) |
| | methylsulfanyl- | | | 5.83 (s, 1 H) 7.27 (d, 2 H) |
| | benzenesulfonamide | | | 7.42-7.53 (m, 3 H) 7.61-7.67 (m, 2 H) 8.26 (s, |
| | trifluoroacetate | | | 1 H) 9.36 (br. s., 1 H) 9.71 (br. s., 1 H) |
| 166 | N-methyl-4-methylsulfanyl-3-[6- | 0.91^{c} | 470.0 (M + H)⁺ | ¹H NMR (400 MHz, DMSO-d₆) δ ppm |
| | (5-oxo-5,6,7,8-tetrahydro- | | | 1.94-2.00 (m, 2 H) 2.40 (d, J = 4.85 Hz, |
| | naphthalen-2-ylamino)- | | | 3 H) 2.47 (s, 3H, and m, 2H |
| | pyrimidin-4-ylamino]- | | | obscured by solvent) 2.81-2.88 (m, 2 |
| | benzenesulfonamide | | | H) 5.90 (s, 1 H) 7.40-7.52 (m, 3 H) |
| | trifluoroacetate | | | 7.56 (s, 1 H) 7.60-7.65 (m, 2 H) |
| | | | | 7.76 (d, J = 8.38 Hz, 1 H) 8.28 (s, 1 H) |
| | | | | 9.12 (br. s., 1 H) 9.71 (br. s., 1 H) |
| 167 | N-methyl-4-methylsulfanyl-3-[6- | 0.99^{c} | 456.0 (M + H)⁺ | ¹H NMR (400 MHz, DMSO-d₆) δ ppm |
| | (3,4,5-trifluoro-phenylamino)- | | | 2.40 (d, J = 4.85 Hz, 3 H) 2.47 (s, 3H, |
| | pyrimidin-4-ylamino]- | | | and m, 2H obscured by solvent) |
| | benzenesulfonamide | | | 5.79 (s, 1 H) 7.44 (q, J = 4.41 Hz, 1 H) |
| | trifluoroacetate | | | 7.47-7.55 (m, 3 H) 7.60-7.66 (m, 2 H) |
| | | | | 8.28 (s, 1 H) 9.19 (br. s., 1 H) 9.70 (s, |
| | | | | 1 H) |
| 168 | N-methyl-3-[6-(4-methyl-2-oxo- | 0.92^{c} | 483.8 (M + H)⁺ | ¹H NMR (400 MHz, DMSO-d₆) δ ppm |
| | 2H-chromen-7-ylamino)- | | | 2.35 (s, 3 H) 2.41 (d, J = 4.85 Hz, 3 H) |
| | pyrimidin-4-ylamino]-4- | | | 2.46 (s, 3H, and m, 2H obscured by |
| | methylsulfanyl- | | | solvent) 5.90 (s, 1 H) 6.16 (s, 1 H) |
| | benzenesulfonamide | | | 7.39 (dd, J = 8.60, 1.98 Hz, 1 H) |
| | trifluoroacetate | | | 7.44 (q, 1 H) 7.49 (d, J = 8.38 Hz, 1 H) |
| | | | | 7.60-7.66 (m, 3 H) 7.90 (d, J = 1.76 Hz, 1 |
| | | | | H) 8.29 (s, 1 H) 9.05 (s, 1 H) 9.76 (s, |
| | | | | 1 H) |
| 169 | 3-[6-(indan-5-ylamino)-pyrimidin- | 0.95^{c} | 441.9 (M + H)⁺ | ¹H NMR (400 MHz, DMSO-d₆) δ ppm |
| | 4-ylamino]-N-methyl-4- | | | 1.98 (quin, J = 7.39 Hz, 2 H) 2.38 (d, |
| | methylsulfanyl- | | | J = 5.29 Hz, 3 H) 2.46 (s, 3H, obscured |
| | benzenesulfonamide | | | by solvent) 2.79 (q, J = 7.94 Hz, 4 H) |
| | trifluoroacetate | | | 5.77 (s, 1 H) 7.11 (s, 1 H) 7.15 (s, 1 |
| | | | | H) 7.28 (s, 1 H) 7.44 (q, J = 4.85 Hz, 1 |
| | | | | H) 7.50 (d, J = 8.82 Hz, 1 H) |
| | | | | 7.61-7.66 (m, 2 H) 8.23 (s, 1 H) 9.38 (br. s., |
| | | | | 1 H) 9.59-9.65 (m, 1 H) |
| 170 | 3-[6-(1H-indazol-6-ylamino)- | 0.79^{c} | 442.0 (M + H)⁺ | ¹H NMR (400 MHz, DMSO-d₆) δ ppm |
| | pyrimidin-4-ylamino]-N-methyl-4- | | | 2.38 (d, J = 4.85 Hz, 3 H) 5.86 (s, 1 H) |
| | methylsulfanyl- | | | 7.03 (dd, J = 8.60, 1.54 Hz, 1 H) |
| | benzenesulfonamide | | | 7.45 (q, J = 4.85 Hz, 1 H) 7.51 (d, J = 8.38 Hz, |
| | trifluoroacetate | | | 1 H) 7.62-7.69 (m, 3 H) 7.88 (br. |
| | | | | s., 1 H) 7.97 (s, 1 H) 8.31 (s, 1 H) |
| | | | | 9.44 (br. s., 1 H) 9.86 (br. s., 1 H) |
| 171 | N-methyl-3-(6-(2-methyl-1,3- | 0.92^{c} | 484.9 (M + H)⁺ | ¹H NMR (400 MHz, DMSO-d₆) δ ppm |
| | dioxoisoindolin-5- | | | 2.42 (d, J = 5.07 Hz, 3 H) 2.47 (s, 3 H, |
| | ylamino)pyrimidin-4-ylamino)-4- | | | obscured by solvent) 2.98 (s, 3 H) |
| | (methylthio)benzenesulfonamide | | | 5.91 (s, 1 H) 7.45-7.49 (m, 1 H) |
| | trifluoroacetate | | | 7.51 (d, J = 9.26 Hz, 1 H) 7.62-7.66 (m, 2 |
| | | | | H) 7.73 (d, J = 8.60 Hz, 1 H) 7.81 (dd, |
| | | | | J = 8.38, 1.98 Hz, 1 H) 8.31 (d, J = 1.54 Hz, |
| | | | | 1 H) 8.34 (s, 1 H) 9.15 (s, 1 H) |
| | | | | 10.00 (s, 1 H) |
| 172 | 3-[6-(3,5-dimethoxy- | 0.89^{c} | 416.0 (M + H)⁺ | ¹H NMR (400 MHz, METHANOL-d₄) δ |
| | phenylamino)-pyrimidin-4- | | | ppm 2.53 (s, 3 H) 3.79 (s, 6 H) |
| | ylamino]-N-methyl- | | | 6.20 (d, J = 0.88 Hz, 1 H) 6.42 (d, J = 4.41 Hz, |
| | benzenesulfonamide | | | 1 H) 6.54 (d, J = 2.20 Hz, 2 H) |
| | trifluoroacetate | | | 7.56-7.61 (m, 1 H) 7.61-7.64 (m, 1 H) |
| | | | | 7.67-7.71 (m, 1 H) 8.02 (d, J = 3.53 Hz, |
| | | | | 1 H) 8.32 (d, J = 0.88 Hz, 1 H) |
| 173 | N-methyl-3-[6-(3,4,5-trimethoxy- | 0.84^{c} | 446.0 (M + H)⁺ | ¹H NMR (400 MHz, METHANOL-d₄) δ |
| | phenylamino)-pyrimidin-4- | | | ppm 2.53 (s, 3 H) 3.76 (s, 3 H) |
| | ylamino]-benzenesulfonamide | | | 3.83 (s, 6 H) 6.12 (s, 1 H) 6.69 (s, 2 H) |
| | trifluoroacetate | | | 7.56-7.61 (m, 1 H) 7.62 (t, J = 1.54 Hz, |
| | | | | 1 H) 7.64 (dd, J = 3.09, 1.32 Hz, 0 |
| | | | | H) 7.68 (m, J = 2.09, 1.43 Hz, 1 H) |
| | | | | 8.00 (t, J = 1.76 Hz, 1 H) 8.32 (d, J = 0.88 Hz, |
| | | | | 1 H) |
| 174 | 3-[6-(3-ethynyl-phenylamino)- | 0.91^{c} | 379.9 (M + H)⁺ | ¹H NMR (400 MHz, DMSO-d₆) δ ppm |
| | pyrimidin-4-ylamino]-N-methyl- | | | 2.42 (d, J = 4.85 Hz, 3 H) 4.18 (s, 1 H) |
| | benzenesulfonamide | | | 6.18 (d, J = 1.10 Hz, 1 H) |
| | trifluoroacetate | | | 7.08-7.14 (m, 1 H) 7.32 (t, J = 7.94 Hz, 1 H) |
| | | | | 7.35-7.38 (m, 1 H) 7.44 (q, J = 4.92 Hz, 1 |
| | | | | H) 7.48-7.55 (m, 2 H) 7.76 (t, J = 1.76 Hz, |
| | | | | 1 H) 7.86 (dt, J = 7.06, 1.21 Hz, 1 |
| | | | | H) 8.04 (t, J = 1.87 Hz, 1 H) 8.39 (s, 1 |
| | | | | H) 9.58 (s, 1 H) 9.78 (s, 1 H) |
| 175 | 3-[6-(benzo[1,3]dioxol-5- | 0.82^{c} | 399.9 (M + H)⁺ | ¹H NMR (400 MHz, METHANOL-d₄) δ |
| | ylamino)-pyrimidin-4-ylamino]-N- | | | ppm 2.53 (s, 3 H) 6.02 (s, 2 H) |
| | methyl-benzenesulfonamide | | | 6.06 (s, 1 H) 6.81 (dd, J = 8.16, 2.21 Hz, 1 |
| | trifluoroacetate | | | H) 6.88-6.92 (m, 2 H) 7.59 (m, |
| | | | | J = 7.72 Hz, 1 H) 7.62-7.67 (m, 2 H) |
| | | | | 7.99 (t, J = 1.76 Hz, 1 H) 8.30 (s, 1 H) |
| 176 | 3-[6-(3-chloro-4-hydroxy- | 0.79^{c} | 406.0 (M + H)⁺ | ¹H NMR (400 MHz, METHANOL-d₄) δ |
| | phenylamino)-pyrimidin-4- | | | ppm 2.53 (s, 3 H) 6.02 (s, 1 H) |
| | ylamino]-N-methyl- | | | 7.00 (d, J = 8.60 Hz, 1 H) 7.13 (dd, J = 8.60, |
| | benzenesulfonamide | | | 2.65 Hz, 1 H) 7.37 (d, J = 2.65 Hz, 1 H) |
| | trifluoroacetate | | | 7.55-7.69 (m, 3 H) 7.99 (d, J = 1.54 Hz, |
| | | | | 1 H) 8.31 (s, 1 H) |
| 177 | 3-[6-(3,4-difluoro-phenylamino)- | 0.92^{c} | 391.9 (M + H)⁺ | ¹H NMR (400 MHz, METHANOL-d₄) δ |
| | pyrimidin-4-ylamino]-N-methyl- | | | ppm 2.54 (s, 3 H) 6.14 (s, 1 H) |
| | benzenesulfonamide | | | 7.17-7.22 (m, 1 H) 7.29-7.38 (m, 1 H) |
| | trifluoroacetate | | | 7.48 (ddd, J = 11.80, 7.06, 2.54 Hz, 1 |
| | | | | H) 7.58-7.69 (m, 3 H) 7.99 (t, J = 1.76 Hz, |
| | | | | 1 H) 8.37 (s, 1 H) |
| 178 | N-methyl-3-[6-(4-piperidin-1-yl- | 0.70^{c} | 439.0 (M + H)⁺ | ¹H NMR (400 MHz, DMSO-d₆) δ ppm |
| | phenylamino)-pyrimidin-4- | | | 1.60 (br. s., 2 H) 1.81 (br. s., 4 H) |
| | ylamino]-benzenesulfonamide | | | 2.41 (d, J = 4.85 Hz, 3 H) 3.37 (br. s., 4 H) |
| | trifluoroacetate | | | 6.17 (br. s., 1 H) 7.32 (d, J = 7.94 Hz, 1 |
| | | | | H) 7.40-7.51 (m, 3 H) 7.54-7.63 (m, |
| | | | | 2 H) 7.86 (d, J = 9.26 Hz, 1 H) 8.07 (s, |
| | | | | 1 H) 8.33 (s, 1 H) 9.49 (br. s., 1 H) |
| | | | | 9.67 (s, 1 H) |
| 179 | 3-[6-(4-cyano-phenylamino)- | 0.93^{c} | 381.0 (M + H)⁺ | ¹H NMR (400 MHz, DMSO-d₆) δ ppm |
| | pyrimidin-4-ylamino]-N-methyl- | | | 2.44 (d, 3H, obscured by solvent) |
| | benzenesulfonamide | | | 6.26 (s, 1 H) 7.33 (d, 1 H) 7.38-7.44 (m, 1 |
| | trifluoroacetate | | | H) 7.46-7.53 (m, 1 H) 7.69 (d, J = 8.82 Hz, |
| | | | | 2 H) 7.82 (d, J = 8.38 Hz, 2 H) |
| | | | | 7.88 (d, J = 9.26 Hz, 1 H) 8.07 (br. s., 1 H) |
| | | | | 8.40 (s, 1 H) 9.71 (s, 1 H) 9.84 (s, 1 |
| | | | | H) |
| 180 | N-methyl-3-[6-(2-methyl-4-oxo- | 0.94^{c} | 437.9 (M + H)⁺ | ¹H NMR (400 MHz, DMSO-d₆) δ ppm |
| | 4H-chromen-7-ylamino)- | | | 2.33 (s, 3 H) 2.40 (d, J = 4.85 Hz, 3 H) |
| | pyrimidin-4-ylamino]- | | | 6.09 (s, 1 H) 6.46 (s, 1 H) 7.31 (d, |
| | benzenesulfonamide | | | J = 7.94 Hz, 1 H) 7.49 (m, J = 4.41 Hz, 4 |
| | trifluoroacetate | | | H) 7.83 (d, J = 8.82 Hz, 1 H) 7.89 (d, |
| | | | | J = 8.38 Hz, 1 H) 8.14 (s, 1 H) 8.28 (d, |
| | | | | J = 1.32 Hz, 1 H) 8.45 (s, 1 H) 9.91 (s, |
| | | | | 1 H) 10.27 (s, 1 H) |
| 181 | 3-[6-(3,5-dichloro-4-hydroxy- | 0.85^{c} | 440.0 (M + H)⁺ | ¹H NMR (400 MHz, DMSO-d₆) δ ppm |
| | phenylamino)-pyrimidin-4- | | | 2.43 (d, J = 5.02 Hz, 3 H) 6.15 (s, 1 H) |
| | ylamino]-N-methyl- | | | 7.32 (d, 1 H) 7.44 (q, J = 5.02 Hz, 1 H) |
| | benzenesulfonamide | | | 7.49 (t, J = 8.03 Hz, 1 H) 7.65 (s, 2 H) |
| | trifluoroacetate | | | 7.89 (dd, J = 8.16, 1.38 Hz, 1 H) |
| | | | | 8.08 (s, 1 H) 8.34 (s, 1 H) 9.41 (s, 1 H) |
| | | | | 9.65 (s, 1 H) 9.67-9.74 (m, 1 H) |
| 182 | N-methyl-3-{6-[3-(2-methyl- | 0.92^{c} | 453.0 (M + H)⁺ | ¹H NMR (400 MHz, DMSO-d₆) δ ppm |
| | thiazol-4-yl)-phenylamino]- | | | 2.27 (d, J = 5.02 Hz, 3 H) 2.55 (s, 3 H) |
| | pyrimidin-4-ylamino}- | | | 6.12 (s, 1 H) 7.15-7.23 (m, 2 H) |
| | benzenesulfonamide | | | 7.27-7.36 (m, 2 H) 7.40 (d, J = 7.78 Hz, 1 |
| | trifluoroacetate | | | H) 7.49 (d, J = 8.03 Hz, 1 H) |
| | | | | 7.70-7.74 (m, 2 H) 7.86 (s, 1 H) 7.93 (s, 1 |
| | | | | H) 8.20 (s, 1 H) 9.41 (s, 1 H) 9.61 (s, |
| | | | | 1 H) |
| 183 | 3-[6-(1H-indazol-5-ylamino)- | 0.66^{c} | 396.0 (M + H)⁺ | ¹H NMR (400 MHz, DMSO-d₆) δ ppm |
| | pyrimidin-4-ylamino]-N-methyl- | | | 2.34 (d, J = 4.77 Hz, 3 H) 6.01 (s, 1 H) |
| | benzenesulfonamide | | | 7.26 (dd, 1 H) 7.32 (d, J = 7.78 Hz, 1 |
| | trifluoroacetate | | | H) 7.37 (q, J = 4.94 Hz, 1 H) |
| | | | | 7.43-7.52 (m, 2 H) 7.73 (d, J = 7.78 Hz, 1 H) |
| | | | | 7.80 (s, 1 H) 7.94 (s, 1 H) 7.99 (s, 1 |
| | | | | H) 8.30 (s, 1 H) 9.57 (br. s., 1 H) |
| | | | | 9.79 (br. s., 1 H) |
| 184 | N-methyl-3-[6-(5-oxo-5,6,7,8- | 0.92^{c} | 424.0 (M + H)⁺ | ¹H NMR (400 MHz, METHANOL-d₄) δ |
| | tetrahydro-naphthalen-2- | | | ppm 2.12 (dt, J = 12.46, 6.34 Hz, 2 H) |
| | ylamino)-pyrimidin-4-ylamino]- | | | 2.55 (s, 3 H) 2.63 (t, 2 H) 2.98 (t, |
| | benzenesulfonamide | | | J = 5.95 Hz, 2 H) 6.32 (s, 1 H) 7.41 (dd, |
| | trifluoroacetate | | | J = 8.60, 2.21 Hz, 1 H) 7.48 (d, J = 2.20 Hz, |
| | | | | 1 H) 7.56-7.64 (m, 2 H) 7.71 (dt, |
| | | | | J = 7.11, 2.18 Hz, 1 H) 7.96 (d, J = 8.60 Hz, |
| | | | | 1 H) 8.03-8.07 (m, 1 H) 8.41 (s, |
| | | | | 1 H) |
| 185 | 3-[6-(4-cyanomethyl- | 0.93^{c} | 395.2 (M + H)⁺ | ¹H NMR (400 MHz, DMSO-d₆) δ ppm |
| | phenylamino)-pyrimidin-4- | | | 2.44 (d, J = 5.02 Hz, 3 H) 3.97 (s, 2 H) |
| | ylamino]-N-methyl- | | | 6.27 (s, 1 H) 7.26-7.34 (m, 3 H) |
| | benzenesulfonamide | | | 7.45-7.53 (m, 2 H) 7.62 (d, J = 8.53 Hz, 2 |
| | trifluoroacetate | | | H) 7.90 (d, J = 9.79 Hz, 1 H) 8.13 (s, 1 |
| | | | | H) 8.34 (s, 1 H) 9.45 (s, 1 H) 9.66 (s, |
| | | | | 1 H) |
| 186 | N-methyl-3-[6-(4-methyl-2-oxo- | 0.93^{c} | 437.9 (M + H)⁺ | ¹H NMR (400 MHz, DMSO-d₆) δ ppm |
| | 2H-chromen-7-ylamino)- | | | 2.37 (s, 3 H) 2.42 (d, J = 4.85 Hz, 3 H) |
| | pyrimidin-4-ylamino]- | | | 6.17 (s, 1 H) 6.30 (s, 1 H) 7.33 (d, |
| | benzenesulfonamide | | | J = 7.94 Hz, 1 H) 7.41-7.54 (m, 3 H) |
| | trifluoroacetate | | | 7.66 (d, J = 8.82 Hz, 1 H) 7.91 (dd, |
| | | | | J = 8.16, 1.54 Hz, 1 H) 7.96 (d, J = 2.20 Hz, |
| | | | | 1 H) 8.08-8.11 (m, 1 H) 8.44 (s, |
| | | | | 1 H) 9.71 (s, 1 H) 9.85 (s, 1 H) |
| 187 | 3-[6-(1-acetyl-2,3-dihydro-1H- | 0.82^{c} | 439.0 (M + H)⁺ | ¹H NMR (400 MHz, DMSO-d₆) δ ppm |
| | indol-6-ylamino)-pyrimidin-4- | | | 2.13 (s, 3 H) 2.40 (d, J = 5.29 Hz, 3 H) |
| | ylamino]-N-methyl- | | | 3.07 (t, J = 8.60 Hz, 2 H) 4.08 (t, |
| | benzenesulfonamide | | | J = 8.38 Hz, 2 H) 6.11 (s, 1 H) 7.16 (d, |
| | trifluoroacetate | | | 1 H) 7.28 (d, J = 7.94 Hz, 1 H) 7.34 (d, |
| | | | | J = 7.94 Hz, 1 H) 7.41 (q, J = 4.85 Hz, 1 |
| | | | | H) 7.49 (t, J = 7.94 Hz, 1 H) 7.81 (d, |
| | | | | J = 8.38 Hz, 1 H) 8.00 (s, 1 H) 8.05 (s, |
| | | | | 1 H) 8.32 (s, 1 H) 9.51 (br. s., 1 H) |
| | | | | 9.76 (br. s., 1 H) |
| 188 | 3-[6-(3-methoxy-5- | 1.01^{c} | 454.0 (M + H)⁺ | ¹H NMR (400 MHz, DMSO-d₆) δ ppm |
| | trifluoromethyl-phenylamino)- | | | 2.41 (d, J = 5.29 Hz, 3 H) 3.79 (s, 3 H) |
| | pyrimidin-4-ylamino]-N-methyl- | | | 6.19 (s, 1 H) 6.79 (s, 1 H) |
| | benzenesulfonamide | | | 7.29-7.34 (m, 1 H) 7.41 (d, 1 H) 7.49 (s, 1 H) |
| | trifluoroacetate | | | 7.51 (s, 1 H) 7.61 (s, 1 H) |
| | | | | 7.87-7.93 (m, 1 H) 8.04 (s, 1 H) 8.38 (s, 1 H) |
| | | | | 9.62 (s, 1 H) 9.66 (s, 1 H) |
| 189 | N-methyl-3-[6-(4-methyl-2-oxo- | 0.84^{c} | 437.0 (M + H)⁺ | ¹H NMR (400 MHz, DMSO-d₆) δ ppm |
| | 1,2-dihydro-quinolin-7-ylamino)- | | | 2.34 (s, 3 H) 2.41 (d, J = 5.29 Hz, 3 H) |
| | pyrimidin-4-ylamino]- | | | 6.19 (s, 1 H) 6.24 (s, 1 H) 7.32 (d, |
| | benzenesulfonamide | | | J = 7.50 Hz, 1 H) 7.37-7.41 (m, 2 H) |
| | trifluoroacetate | | | 7.49 (t, J = 7.94 Hz, 2 H) 7.59 (d, |
| | | | | J = 8.82 Hz, 1 H) 7.64 (s, 1 H) 7.88 (d, |
| | | | | J = 7.94 Hz, 2 H) 8.05 (s, 1 H) 8.37 (s, |
| | | | | 2 H) 9.63 (br. s., 3 H) 11.48 (br. s., 2 |
| | | | | H) |
| 190 | N-methyl-3-[6-(3,4,5-trifluoro- | 0.99^{c} | 410.0 (M + H)⁺ | ¹H NMR (400 MHz, DMSO-d₆) δ ppm |
| | phenylamino)-pyrimidin-4- | | | 2.41 (s, 3 H) 6.34 (s, 1 H) |
| | ylamino]-benzenesulfonamide | | | 7.47-7.62 (m, 4 H) 7.81 (d, J = 9.26 Hz, 1 H) |
| | hydrochloride | | | 8.00 (s, 1 H) 8.44 (s, 1 H) 10.19 (s, 1 H) |
| | | | | 10.31 (s, 1 H) |
| 191 | 3-[6-(indan-5-ylamino)-pyrimidin- | 0.93^{c} | 395.9 (M + H)⁺ | ¹H NMR (400 MHz, DMSO-d₆) δ ppm |
| | 4-ylamino]-N-methyl- | | | 2.03 (quin, J = 7.40 Hz, 2 H) 2.44 (d, |
| | benzenesulfonamide | | | J = 5.02 Hz, 3 H) 2.79-2.91 (m, 4 H) |
| | trifluoroacetate | | | 6.13 (s, 1 H) 7.16-7.24 (m, 2 H) |
| | | | | 7.35-7.41 (m, 2 H) 7.46 (q, J = 4.60 Hz, 1 |
| | | | | H) 7.54 (t, J = 8.03 Hz, 1 H) 7.85 (d, |
| | | | | J = 8.03 Hz, 1 H) 8.04 (s, 1 H) 8.36 (s, |
| | | | | 1 H) 9.45 (br. s., 1 H) 9.79 (br. s., 1 H) |
| 192 | 3-[6-(4-chloro-phenylamino)- | 1.26^{c} | 495.9 (M + H)⁺ | ¹H NMR (400 MHz, DMSO-d₆) δ ppm |
| | pyrimidin-4-ylamino]-N-methyl-4- | | | 1.18 (d, J = 6.78 Hz, 6 H) 2.50 (d, 3H, |
| | (propane-2-sulfonyl)- | | | obscured by solvent) 3.52 (spt, 1H, |
| | benzenesulfonamide | | | obscured by solvent) 6.32 (s, 1 H) |
| | | | | 7.37 (d, J = 8.78 Hz, 2 H) 7.65 (d, |
| | | | | J = 8.78 Hz, 3 H) 7.80 (q, J = 4.68 Hz, 1 |
| | | | | H) 8.05 (d, J = 8.28 Hz, 1 H) 8.36 (s, 1 |
| | | | | H) 8.51 (s, 1 H) 9.00 (s, 1 H) 9.57 (s, |
| | | | | 1 H) |
| 193 | 3-(6-(3-bromo-5- | 1.12^{c} | 527.8 (M + H)⁺ | ¹H NMR (400 MHz, DMSO-d₆) δ ppm |
| | methylphenylamino)pyrimidin-4- | | | 2.26 (s, 3 H) 2.47 (d, 3H, obscured by |
| | ylamino)-N-methyl-4- | | | solvent) 3.28 (s, 3 H) 6.32 (s, 1 H) |
| | (methylsulfonyl)benzenesulfonamide | | | 6.96-7.00 (m, 1 H) 7.32 (s, 1 H) |
| | | | | 7.64 (dd, J = 8.38, 1.76 Hz, 1 H) |
| | | | | 7.78-7.84 (m, 2 H) 8.08 (d, J = 8.16 Hz, 1 H) |
| | | | | 8.34 (s, 1 H) 8.39 (d, J = 1.54 Hz, 1 H) |
| | | | | 8.92 (s, 1 H) 9.56 (s, 1 H) |
| 194 | 3-(6-(1H-indol-6- | 0.89^{c} | 472.9 (M + H)⁺ | ¹H NMR (400 MHz, DMSO-d₆) δ ppm |
| | ylamino)pyrimidin-4-ylamino)-N- | | | 2.45 (d, J = 5.07 Hz, 3 H) 3.27 (s, 3 H) |
| | methyl-4-(methylsulfonyl)benzenesulfonamide | | | 6.22 (s, 1 H) 6.39 (t, 1 H) 6.98 (dd, |
| | | | | J = 8.49, 1.87 Hz, 1 H) 7.31 (t, J = 2.76 Hz, |
| | | | | 1 H) 7.51 (d, J = 8.38 Hz, 1 H) |
| | | | | 7.60 (br. s., 1 H) 7.68-7.74 (m, 1 H) |
| | | | | 7.77 (q, J = 4.92 Hz, 1 H) 8.10 (d, J = 8.38 Hz, |
| | | | | 1 H) 8.28 (s, 1 H) 8.32 (s, 1 H) |
| | | | | 9.23 (br. s., 1 H) 9.69 (br. s., 1 H) |
| | | | | 11.09 (br. s., 1 H) |
| 195 | 3-(6-(3- | 0.98^{c} | 458.0 (M + H)⁺ | ¹H NMR (400 MHz, DMSO-d₆) δ ppm |
| | ethynylphenylamino)pyrimidin-4- | | | 3.27 (s, 3 H) 4.15 (s, 1 H) 6.28 (s, 1 |
| | ylamino)-N-methyl-4- | | | H) 7.07 (d, J = 7.72 Hz, 1 H) 7.29 (t, |
| | (methylsulfonyl)benzenesulfonamide | | | J = 8.05 Hz, 1 H) 7.54 (dd, J = 7.83, |
| | | | | 1.65 Hz, 1 H) 7.63 (dd, J = 8.38, 1.76 Hz, |
| | | | | 1 H) 7.73-7.80 (m, 2 H) 8.07 (d, |
| | | | | J = 8.38 Hz, 1 H) 8.33 (s, 1 H) 8.40 (d, |
| | | | | J = 1.54 Hz, 1 H) 8.93 (s, 1 H) 9.50 (s, |
| | | | | 1 H) |
| 196 | 3-[6-(indan-5-ylamino)-pyrimidin- | 1.15^{c} | 473.9 (M + H)⁺ | ¹H NMR (300 MHz, DMSO-d₆) δ ppm |
| | 4-ylamino]-4-methanesulfonyl- | | | 1.99 (quin, J = 7.35 Hz, 2 H) 2.47 (d, |
| | N-methyl-benzenesulfonamide | | | 3H, obscured by solvent) |
| | | | | 2.74-2.88 (m, 4 H) 3.28 (s, 3 H) 6.23 (s, 1 H) |
| | | | | 7.11-7.25 (m, 2 H) 7.38 (s, 1 H) |
| | | | | 7.76 (d, J = 4.90 Hz, 2 H) 8.08 (d, J = 8.29 Hz, |
| | | | | 1 H) 8.29 (s, 1 H) 8.38 (s, 1 H) |
| | | | | 8.97 (br. s., 1 H) 9.40 (br. s., 1 H) |
| 197 | 3-[6-(benzothiazol-6-ylamino)- | 1.02^{c} | 490.9 (M + H)⁺ | ¹H NMR (300 MHz, DMSO-d₆) δ ppm |
| | pyrimidin-4-ylamino]-4- | | | 2.47 (d, 3H, obscured by solvent) |
| | methanesulfonyl-N-methyl- | | | 3.29 (s, 3 H) 6.34 (s, 1 H) 7.58 (dd, 1 |
| | benzenesulfonamide | | | H) 7.68 (dd, J = 8.29, 1.88 Hz, 1 H) |
| | | | | 7.78 (q, J = 5.02 Hz, 1 H) 8.01 (d, |
| | | | | J = 8.67 Hz, 1 H) 8.10 (d, J = 8.29 Hz, 1 |
| | | | | H) 8.37 (s, 1 H) 8.39 (d, J = 1.88 Hz, 1 |
| | | | | H) 8.49 (d, J = 1.88 Hz, 1 H) 9.05 (br. |
| | | | | s., 1 H) 9.24 (s, 1 H) 9.78 (s, 1 H) |
| 198 | 4-methanesulfonyl-N-methyl-3- | 1.14^{c} | 502.0 (M + H)⁺ | ¹H NMR (300 MHz, DMSO-d₆) δ ppm |
| | [6-(5-oxo-5,6,7,8-tetrahydro- | | | 1.93-2.04 (m, 2 H) 2.47 (d, 3H, and t, |
| | naphthalen-2-ylamino)- | | | 2H, obscured by solvent) 2.88 (t, |
| | pyrimidin-4-ylamino]- | | | J = 5.46 Hz, 2 H) 3.29 (s, 3 H) 6.38 (s, |
| | benzenesulfonamide | | | 1 H) 7.57 (dd, J = 8.67, 1.88 Hz, 1 H) |
| | | | | 7.62 (s, 1 H) 7.68 (dd, J = 8.29, 1.51 Hz, |
| | | | | 1 H) 7.75-7.83 (m, 2 H) 8.11 (d, |
| | | | | J = 8.29 Hz, 1 H) 8.40 (s, 2 H) 9.05 (br. |
| | | | | s., 1 H) 9.81 (s, 1 H) |
| 199 | N-methyl-3-(6-(2- | 0.93^{c} | 505.1 (M + H)⁺ | ¹H NMR (400 MHz, DMSO-d₆) δ ppm |
| | methylbenzo[d]thiazol-5- | | | 2.50 (d, 3H) 2.79 (s, 3 H) 3.32 (s, 3 |
| | ylamino)pyrimidin-4-ylamino)-4- | | | H) 6.38 (s, 1 H) 7.53 (dd, J = 8.66, 1.88 Hz, |
| | (methylsulfonyl)benzenesulfonamide | | | 1 H) 7.67 (dd, J = 8.28, 1.76 Hz, 1 |
| | | | | H) 7.81 (q, J = 4.94 Hz, 1 H) 7.94 (d, 1 |
| | | | | H) 8.11 (d, J = 8.28 Hz, 1 H) 8.28 (d, |
| | | | | J = 1.51 Hz, 1 H) 8.37 (s, 1 H) 8.46 (s, |
| | | | | 1 H) 8.96 (s, 1 H) 9.64 (s, 1 H) |
| 200 | N-methyl-4-(methylsulfonyl)-3- | 0.81^{c} | 515.0 (M + H)⁺ | ¹H NMR (400 MHz, DMSO-d₆) δ ppm |
| | [(6-{[4-(1H-1,2,4-triazol-1- | | | 2.47 (d, 3H, obscured by solvent) |
| | ylmethyl)phenyl]amino}-4- | | | 3.26 (s, 3 H) 5.31 (s, 2 H) 6.30 (s, 1 H) |
| | pyrimidinyl)amino]benzenesulfonamide | | | 7.22 (d, J = 8.60 Hz, 2 H) 7.51 (d, |
| | | | | J = 8.16 Hz, 2 H) 7.60 (dd, J = 8.27, |
| | | | | 1.43 Hz, 1 H) 7.72-7.79 (m, 1 H) |
| | | | | 7.94 (s, 1 H) 8.05 (d, J = 8.38 Hz, 1 H) |
| | | | | 8.27 (s, 1 H) 8.40 (s, 1 H) 8.60 (s, 1 |
| | | | | H) 8.82 (br. s., 1 H) 9.45 (s, 1 H) |
| 201 | 3-[6-(1H-indol-5-ylamino)- | 0.97^{c} | 472.9 (M + H)⁺ | ¹H NMR (300 MHz, DMSO-d₆) δ ppm |
| | pyrimidin-4-ylamino]-4- | | | 2.45 (d, 3H, obscured by solvent) |
| | methanesulfonyl-N-methyl- | | | 3.26 (s, 3 H) 6.15 (s, 1 H) 6.44 (br. s., 1 H) |
| | benzenesulfonamide | | | 7.05 (dd, J = 8.67, 1.88 Hz, 1 H) |
| | | | | 7.38 (t, J = 2.64 Hz, 1 H) 7.43 (d, J = 8.67 Hz, |
| | | | | 1 H) 7.56 (s, 1 H) 7.73-7.84 (m, 2 H) |
| | | | | 8.08-8.19 (m, 2 H) 8.33 (s, 1 H) |
| | | | | 9.46 (br. s., 1 H) 9.93 (br. s., 1 H) |
| | | | | 11.21 (br. s., 1 H) |
| 202 | 4-methanesulfonyl-N-methyl-3- | 0.98^{c} | 516.0 (M + H)⁺ | ¹H NMR (400 MHz, DMSO-d₆) δ ppm |
| | [6-(2-methyl-4-oxo-4H-chromen- | | | 2.35 (s, 3 H) 2.50 (d, 3H, obscured by |
| | 7-ylamino)-pyrimidin-4-ylamino]- | | | solvent) 3.29 (s, 3 H) 6.12 (d, J = 0.66 Hz, |
| | benzenesulfonamide | | | 1 H) 6.39 (s, 1 H) 7.40 (dd, |
| | | | | J = 8.60, 1.98 Hz, 1 H) 7.68 (dd, |
| | | | | J = 8.38, 1.76 Hz, 1 H) 7.75-7.81 (m, |
| | | | | 1 H) 7.87 (d, J = 8.82 Hz, 1 H) 8.11 (d, |
| | | | | J = 8.16 Hz, 1 H) 8.25 (d, J = 1.98 Hz, 1 |
| | | | | H) 8.41 (d, J = 1.76 Hz, 1 H) 8.45 (s, 1 |
| | | | | H) 9.05 (s, 1 H) 9.98 (s, 1 H) |
| 203 | 5-({6-[(4-chlorophenyl)amino]-4- | 2.23^{a} | 408.0 (M + H)⁺ | ¹H NMR (400 MHz, METHANOL-d₄) δ |
| | pyrimidinyl}amino)-2-fluoro-N- | | | ppm 2.52 (s, 3 H) 6.01 (s, 1 H) 7.15 (t, |
| | methylbenzenesulfonamide | | | J = 9.29 Hz, 1 H) 7.20 (d, J = 8.78 Hz, 2 |
| | | | | H) 7.36 (d, J = 9.03 Hz, 2 H) |
| | | | | 7.65-7.70 (m, 1 H) 7.92-7.95 (m, 1 H) |
| | | | | 8.14-8.15 (m, 1 H) |
| 204 | 5-(6-(4- | 1.01^{c} | 520.0 (M + H)⁺ | ¹H NMR (400 MHz, DMSO-d₆) δ ppm |
| | chlorophenylamino)pyrimidin-4- | | | 1.40 (d, J = 6.17 Hz, 3 H) 2.47 9d, 3H, |
| | ylamino)-2-fluoro-N-methyl-4- | | | obscured by solvent) 5.36-5.46 (m, 1 |
| | (1,1,1-trifluoropropan-2- | | | H) 5.99 (s, 1 H) 7.34 (d, J = 8.82 Hz, 2 |
| | yloxy)benzenesulfonamide | | | H) 7.47-7.60 (m, 3 H) 7.67 (m, |
| | | | | J = 4.85 Hz, 1 H) 7.92 (d, J = 7.72 Hz, 1 |
| | | | | H) 8.25 (s, 1 H) 8.89 (br. s., 1 H) |
| | | | | 9.51 (br. s., 1 H) |
| 205 | 1-{6-[(4-chlorophenyl)amino]-4- | 2.28^{a} | 415.9 (M + H)⁺ | ¹H NMR (400 MHz, DMSO-d6) δ ppm |
| | pyrimidinyl}-N-methyl-2,3- | | | 10.08 (br. s., 1H), 8.76 (s, 1H), |
| | dihydro-1H-indole-6- | | | 8.51 (s, 1H), 7.66 (d, J = 9.03 Hz, 2H), |
| | sulfonamide hydrochloride | | | 7.32-7.52 (m, 5H), 6.16 (s, 1H), 4.07 (t, J = 8.66 Hz, |
| | | | | 2H), 3.30 (t, J = 8.53 Hz, |
| | | | | 2H), 2.42 (s, 3H) |
| 206 | 3-[(6-{[3,4- | 1.83^{a} | 416.2 (M + H)⁺ | ¹H NMR (500 MHz, DMSO-d6) δ ppm |
| | bis(methyloxy)phenyl]amino}-4- | | | 9.76 (br. s., 1H), 9.36 (br. s., 1H), |
| | pyrimidinyl)amino]-N- | | | 8.33 (s, 1H), 8.02 (s, 1H), 7.83 (d, J = 8.06 Hz, |
| | methylbenzenesulfonamide | | | 1H), 7.53 (t, J = 7.93 Hz, 1H), |
| | trifluoroacetate | | | 7.43 (q, J = 4.64 Hz, 1H), 7.38 (d, J = 7.57 Hz, |
| | | | | 1H), 7.05-7.08 (m, 1H), |
| | | | | 6.93-7.01 (m, 2H), 6.09 (s, 1H), |
| | | | | 3.76 (s, 3H), 3.75 (s, 3H), 2.43 (d, J = 4.88 Hz, |
| | | | | 3H) |
| 207 | 3-({6-[(3,4- | 2.36^{a} | 424.0 (M + H)⁺ | ¹H NMR (500 MHz, DMSO-d6) δ ppm |
| | dichlorophenyl)amino]-4- | | | 9.64 (s, 1H), 9.58 (s, 1H), 8.41 (s, |
| | pyrimidinyl}amino)-N- | | | 1H), 8.06-8.10 (m, 2H), |
| | methylbenzenesulfonamide | | | 7.89-7.93 (m, 1H), 7.48-7.56 (m, 3H), 7.41 (q, |
| | trifluoroacetate | | | J = 4.64 Hz, 1H), 7.35 (d, J = 7.81 Hz, |
| | | | | 1H), 6.19 (s, 1H), 2.44 (d, J = 4.88 Hz, |
| | | | | 3H) |
| 208 | 3-({6-[(3,4- | 2.07^{a} | 384.2 (M + H)⁺ | ¹H NMR (500 MHz, DMSO-d6) δ ppm |
| | dimethylphenyl)amino]-4- | | | 9.74 (br. s., 1H), 9.36 (br. s., 1H), |
| | pyrimidinyl}amino)-N- | | | 8.34 (s, 1H), 8.03 (s, 1H), 7.84 (d, J = 8.06 Hz, |
| | methylbenzenesulfonamide | | | 1H), 7.53 (t, J = 7.93 Hz, 1H), |
| | trifluoroacetate | | | 7.42 (q, J = 4.80 Hz, 1H), 7.38 (d, J = 7.81 Hz, |
| | | | | 1H), 7.18-7.24 (m, 2H), |
| | | | | 7.12 (d, J = 8.06 Hz, 1H), 6.11 (s, 1H), |
| | | | | 2.43 (d, J = 4.88 Hz, 3H), 2.22 (s, 3H), |
| | | | | 2.20 (s, 3H) |
| 209 | N-methyl-3-[(6-{[3-(1- | 2.13^{a} | 398.0 (M + H)⁺ | ¹H NMR (500 MHz, DMSO-d6) δ ppm |
| | methylethyl)phenyl]amino}-4- | | | 9.49 (br. s., 1H), 9.15 (br. s., 1H), |
| | pyrimidinyl)amino]benzenesulfonamide | | | 8.31 (s, 1H), 8.08 (s, 1H), 7.90 (d, J = 7.32 Hz, |
| | | | | 1H), 7.46-7.53 (m, 1H), 7.44 (d, |
| | | | | J = 7.81 Hz, 1H), 7.39 (q, J = 4.80 Hz, |
| | | | | 1H), 7.29-7.34 (m, 2H), 7.22 (t, J = 7.81 Hz, |
| | | | | 1H), 6.89 (d, J = 7.57 Hz, |
| | | | | 1H), 6.18 (s, 1H), 2.86 (dt, J = 6.93, |
| | | | | 13.73 Hz, 1H), 2.44 (d, J = 5.13 Hz, |
| | | | | 3H), 1.22 (s, 3H), 1.20 (s, 3H) |
| 210 | 3-[(6-{[3-(1,1- | 2.25^{a} | 412.2 (M + H)⁺ | ¹H NMR (500 MHz, DMSO-d6) δ ppm |
| | dimethylethyl)phenyl]amino}-4- | | | 9.72 (br. s., 1H), 9.42 (br. s., 1H), |
| | pyrimidinyl)amino]-N- | | | 8.36 (s, 1H), 8.03 (s, 1H), 7.86 (d, J = 8.06 Hz, |
| | methylbenzenesulfonamide | | | 1H), 7.53 (t, J = 7.93 Hz, 1H), |
| | trifluoroacetate | | | 7.35-7.46 (m, 4H), 7.28 (t, J = 7.81 Hz, |
| | | | | 1H), 7.11 (d, J = 7.81 Hz, 1H), |
| | | | | 6.16 (s, 1H), 2.44 (d, J = 4.88 Hz, 3H), |
| | | | | 1.29 (s, 9H) |
| 211 | 3-[(6-{[3- | 1.99^{a} | 400.0 (M + H)⁺ | ¹H NMR (500 MHz, DMSO-d6) δ ppm |
| | (ethyloxy)phenyl]amino}-4- | | | 9.60 (s, 1H), 9.29 (br. s., 1H), 8.35 (s, |
| | pyrimidinyl)amino]-N- | | | 1H), 8.07 (s, 1H), 7.86-7.91 (m, 1H), |
| | methylbenzenesulfonamide | | | 7.51 (t, J = 7.93 Hz, 1H), |
| | trifluoroacetate | | | 7.38-7.43 (m, 1H), 7.34 (d, J = 7.81 Hz, 1H), |
| | | | | 7.17-7.23 (m, 2H), 7.03-7.09 (m, |
| | | | | 1H), 6.56-6.61 (m, 1H), 6.21 (s, 1H), |
| | | | | 4.01 (q, J = 6.84 Hz, 2H), 2.44 (d, J = 4.88 Hz, |
| | | | | 3H), 1.33 (t, J = 6.96 Hz, 3H) |
| 212 | 3-({6-[(4-fluorophenyl)amino]-4- | 1.95^{a} | 374.1 (M + H)⁺ | ¹H NMR (500 MHz, DMSO-d6) δ ppm |
| | pyrimidinyl}amino)-N- | | | 9.69 (br. s., 1H), 9.42 (br. s., 1H), |
| | methylbenzenesulfonamide | | | 8.35 (s, 1H), 8.05 (s, 1H), 7.85 (d, J = 8.06 Hz, |
| | trifluoroacetate | | | 1H), 7.50-7.57 (m, 3H), 7.42 (q, |
| | | | | J = 4.80 Hz, 1H), 7.37 (d, J = 7.81 Hz, |
| | | | | 1H), 7.18 (t, J = 8.79 Hz, 2H), 6.12 (s, |
| | | | | 1H), 2.44 (d, J = 4.88 Hz, 3H) |
| 213 | N-methyl-3-[(6-{[3-(1- | 2.08^{a} | 425.2 (M + H)⁺ | ¹H NMR (500 MHz, DMSO-d6) δ ppm |
| | pyrrolidinyl)phenyl]amino}-4- | | | 9.87 (br. s., 1H), 9.48 (br. s., 1H), |
| | pyrimidinyl)amino]benzenesulfonamide | | | 8.36 (s, 1H), 8.01 (s, 1H), 7.83 (d, J = 7.81 Hz, |
| | trifluoroacetate | | | 1H), 7.54 (t, J = 7.93 Hz, 1H), |
| | | | | 7.38-7.45 (m, 2H), 7.15 (t, J = 7.93 Hz, |
| | | | | 1H), 6.70 (d, J = 7.81 Hz, 1H), |
| | | | | 6.58 (s, 1H), 6.34 (d, J = 8.30 Hz, 1H), |
| | | | | 6.21 (s, 1H), 3.22 (t, J = 6.23 Hz, 4H), |
| | | | | 2.43 (d, J = 4.88 Hz, 3H), 1.96 (t, J = 6.35 Hz, |
| | | | | 4H) |
| 214 | N-methyl-3-[(6-{[3-(4-methyl-1- | 0.66^{a} | 454.0 (M + H)⁺ | ¹H NMR (400 MHz, DMSO-d6) δ ppm |
| | piperazinyl)phenyl]amino}-4- | | | 9.58 (m, 2H), 9.24 (s, 1H), 8.34 (s, |
| | pyrimidinyl)amino]benzenesulfonamide | | | 1H), 8.06-8.10 (m, 1H), 7.91 (dd, J = 2.01, |
| | trifluoroacetate | | | 7.78 Hz, 1H), 7.52 (t, J = 7.91 Hz, |
| | | | | 1H), 7.44 (q, J = 4.85 Hz, 1H), |
| | | | | 7.34 (d, J = 7.78 Hz, 1H), |
| | | | | 7.17-7.25 (m, 2H), 7.07 (d, J = 8.28 Hz, 1H), |
| | | | | 6.67-6.74 (m, 1H), 6.19 (s, 1H), |
| | | | | 3.76-3.85 (m, 2H), 3.50-3.58 (m, 2H), |
| | | | | 3.12-3.25 (m, 2H), 2.91-3.03 (m, |
| | | | | 2H), 2.88 (d, J = 4.77 Hz, 3H), |
| | | | | 2.44 (d, J = 5.02 Hz, 3H) |
| 215 | 3-({6-[(3,5- | 2.49^{a} | 423.8 (M + H)⁺ | ¹H NMR (400 MHz, DMSO-d6) δ ppm |
| | dichlorophenyl)amino]-4- | | | 9.71 (s, 1H), 9.68 (s, 1H), 8.45 (s, |
| | pyrimidinyl}amino)-N- | | | 1H), 8.09 (t, J = 1.76 Hz, 1H), |
| | methylbenzenesulfonamide | | | 7.91-7.97 (m, 1H), 7.77 (s, 1H), 7.76 (s, |
| | trifluoroacetate | | | 1H), 7.54 (t, J = 7.91 Hz, 1H), 7.46 (q, |
| | | | | J = 4.94 Hz, 1H), 7.36 (d, J = 8.03 Hz, |
| | | | | 1H), 7.14 (t, J = 1.76 Hz, 1H), 6.21 (s, |
| | | | | 1H), 2.45 (d, J = 5.02 Hz, 3H) |
| 216 | N-methyl-3-({6-[(2-oxo-2,3- | 1.63^{a} | 410.9 (M + H)⁺ | ¹H NMR (400 MHz, DMSO-d6) δ ppm |
| | dihydro-1H-indol-5-yl)amino]-4- | | | 10.39 (s, 1H), 9.78 (br. s., 1H), |
| | pyrimidinyl}amino)benzenesulfonamide | | | 9.42 (br. s., 1H), 8.34 (s, 1H), 8.03 (s, 1H), |
| | trifluoroacetate | | | 7.83 (d, J = 8.03 Hz, 1H), 7.54 (t, J = 7.91 Hz, |
| | | | | 1H), 7.46 (q, J = 4.85 Hz, |
| | | | | 1H), 7.36-7.42 (m, 2H), |
| | | | | 7.18-7.25 (m, 1H), 6.82 (d, J = 8.28 Hz, 1H), |
| | | | | 6.05 (s, 1H), 3.51 (s, 2H), 2.44 (d, J = 5.02 Hz, |
| | | | | 3H) |
| 217 | N-methyl-3-({6-[(2-oxo-2,3- | 1.70^{a} | 413.0 (M + H)⁺ | ¹H NMR (400 MHz, DMSO-d6) δ ppm |
| | dihydro-1,3-benzoxazol-6- | | | 11.56 (s, 1H), 9.62 (s, 1H), 9.37 (s, |
| | yl)amino]-4- | | | 1H), 8.34 (s, 1H), 8.08 (t, J = 2.13 Hz, |
| | pyrimidinyl}amino)benzenesulfonamide | | | 1H), 7.85-7.91 (m, 1H), |
| | trifluoroacetate | | | 7.63-7.67 (m, 1H), 7.52 (t, J = 7.91 Hz, 1H), |
| | | | | 7.44 (q, J = 5.02 Hz, 1H), 7.35 (dt, J = 1.19, |
| | | | | 7.91 Hz, 1H), 7.19 (dd, J = 2.13, |
| | | | | 8.41 Hz, 1H), 7.06 (d, J = 8.28 Hz, |
| | | | | 1H), 6.13 (s, 1H), 2.44 (d, J = 5.02 Hz, |
| | | | | 3H) |
| 218 | N-methyl-3-({6-[(2-oxo-2,3- | 1.58^{a} | 411.9 (M + H)⁺ | ¹H NMR (400 MHz, DMSO-d6) δ ppm |
| | dihydro-1H-benzimidazol-5- | | | 10.56 (s, 1H), 10.51 (s, 1H), 9.48 (s, |
| | yl)amino]-4- | | | 1H), 9.06 (br. s., 1H), 8.28 (s, 1H), |
| | pyrimidinyl}amino)benzenesulfonamide | | | 8.08-8.11 (m, 1H), 7.84-7.90 (m, |
| | trifluoroacetate | | | 1H), 7.49 (t, J = 8.03 Hz, 1H), |
| | | | | 7.39-7.46 (m, 1H), 7.31 (d, J = 8.03 Hz, |
| | | | | 1H), 7.26 (s, 1H), 6.93-6.98 (m, 1H), |
| | | | | 6.88 (d, J = 8.28 Hz, 1H), 6.07 (s, 1H), |
| | | | | 2.44 (d, J = 5.02 Hz, 3H) |
| 219 | N-methyl-3-({6-[(2-oxo-1,2,3,4- | 1.77^{a} | 424.9 (M + H)⁺ | ¹H NMR (400 MHz, DMSO-d6) δ ppm |
| | tetrahydro-7-quinolinyl)amino]-4- | | | 10.13 (s, 1H), 9.72 (s, 1H), 9.42 (br. |
| | pyrimidinyl}amino)benzenesulfonamide | | | s., 1H), 8.35 (s, 1H), 8.06 (s, 1H), |
| | trifluoroacetate | | | 7.83-7.89 (m, 1H), 7.53 (t, J = 8.03 Hz, |
| | | | | 1H), 7.45 (q, J = 4.77 Hz, 1H), |
| | | | | 7.37 (d, J = 7.78 Hz, 1H), |
| | | | | 7.06-7.16 (m, 2H), 7.02 (s, 1H), 6.16 (s, 1H), |
| | | | | 2.84 (t, J = 7.53 Hz, 2H), |
| | | | | 2.42-2.48 (m, 5H) |
| 220 | 3-({6-[(3-bromo-5- | 1.96^{a} | 470.0 (M + H)⁺ | ¹H NMR (500 MHz, DMSO-d6) δ ppm |
| | chlorophenyl)amino]-4- | | | 9.67 (s, 1H), 9.62 (s, 1H), 8.43 (s, |
| | pyrimidinyl}amino)-N- | | | 1H), 8.07 (s, 1H), 7.93 (d, J = 7.81 Hz, |
| | methylbenzenesulfonamide | | | 1H), 7.87 (s, 1H), 7.81 (s, 1H), 7.53 (t, |
| | trifluoroacetate | | | J = 7.93 Hz, 1H), 7.42 (q, J = 4.88 Hz, |
| | | | | 1H), 7.36 (d, J = 7.57 Hz, 1H), 7.24 (s, |
| | | | | 1H), 6.20 (s, 1H), 2.45 (d, J = 4.88 Hz, |
| | | | | 3H) |
| 221 | 3-({6-[(3,5- | 1.49^{a} | 384.2 (M + H)⁺ | ¹H NMR (500 MHz, DMSO-d6) δ ppm |
| | dimethylphenyl)amino]-4- | | | 9.72 (br. s., 1H), 9.34 (br. s., 1H), |
| | pyrimidinyl}amino)-N- | | | 8.36 (s, 1H), 8.03 (s, 1H), 7.86 (d, J = 7.81 Hz, |
| | methylbenzenesulfonamide | | | 1H), 7.53 (t, J = 7.93 Hz, 1H), |
| | trifluoroacetate | | | 7.42 (q, J = 4.56 Hz, 1H), 7.38 (d, J = 7.57 Hz, |
| | | | | 1H), 7.11 (s, 2H), 6.72 (s, |
| | | | | 1H), 6.16 (s, 1H), 2.44 (d, J = 4.64 Hz, |
| | | | | 3H), 2.26 (s, 6H) |
| 222 | N-methyl-3-{[6-({4- | 1.33^{a} | 449.1 (M + H)⁺ | ¹H NMR (500 MHz, DMSO-d6) δ ppm |
| | [(methylamino)sulfonyl]phenyl}amino)- | | | 9.72 (s, 1H), 9.65 (s, 1H), 8.42 (s, |
| | 4- | | | 1H), 8.09 (s, 1H), 7.92 (d, J = 8.1 Hz, |
| | pyrimidinyl]amino}benzenesulfonamide | | | 1H), 7.82 (d, J = 8.8 Hz, 2H), 7.69 (d, |
| | trifluoroacetate | | | J = 8.6 Hz, 2H), 7.52 (t, J = 7.9 Hz, |
| | | | | 1H), 7.41 (q, J = 4.9 Hz, 1H), 7.35 (d, |
| | | | | J = 7.8 Hz, 1H), 7.24 (q, J = 5.0 Hz, |
| | | | | 1H), 6.28 (s, 1H), 2.45 (d, J = 4.9 Hz, |
| | | | | 3H), 2.40 (d, J = 4.6 Hz, 3H) |
| 223 | N-methyl-3-[(6-{[3-(1- | 1.56^{a} | 439.0 (M + H)⁺ | ¹H NMR (400 MHz, METHANOL-d4) δ |
| | pyrrolidinylmethyl)phenyl]amino}- | | | ppm 8.37-8.41 (m, 1H), 8.07 (d, J = 1.76 Hz, |
| | 4- | | | 1H), 7.74 (s, 1H), |
| | pyrimidinyl)amino]benzenesulfonamide | | | 7.67-7.72 (m, 1H), 7.58-7.65 (m, 2H), |
| | trifluoroacetate | | | 7.47-7.58 (m, 2H), 7.35 (d, J = 6.27 Hz, |
| | | | | 1H), 6.26 (s, 1H), 4.42 (s, 2H), |
| | | | | 3.43-3.65 (m, 2H), 3.11-3.29 (m, 2H), |
| | | | | 2.54-2.61 (m, 3H), 2.00-2.25 (m, |
| | | | | 4H) |
| 224 | N-methyl-3-({6-[(4-{[2-(4- | 1.44^{a} | 485.0 (M + H)⁺ | ¹H NMR (400 MHz, DMSO-d6) δ |
| | morpholinyl)ethyl]oxy}phenyl)amino]- | | | ppm 10.35 (br. s., 1H), 9.65 (br. s., |
| | 4- | | | 1H), 9.29 (br. s., 1H), 8.32 (s, 1H), |
| | pyrimidinyl}amino)benzenesulfonamide | | | 8.08 (s, 1H), 7.81-7.89 (m, 1H), |
| | trifluoroacetate | | | 7.43-7.55 (m, 4H), 7.35 (d, J = 7.78 Hz, |
| | | | | 1H), 7.01 (d, J = 9.03 Hz, 2H), 6.11 (s, |
| | | | | 1H), 4.36 (t, J = 4.89 Hz, 2H), |
| | | | | 3.95-4.05 (m, 2H), 3.75 (t, J = 12.05 Hz, |
| | | | | 2H), 3.47-3.62 (m, 4H), |
| | | | | 3.15-3.28 (m, 2H), 2.44 (d, J = 5.02 Hz, 3H) |
| 225 | 3-({6-[(4-{[2- | 1.58^{a} | 443.0 (M + H)⁺ | ¹H NMR (400 MHz, DMSO-d6) δ ppm |
| | (dimethylamino)ethyl]oxy}phenyl)amino]- | | | 10.09 (br. s., 1H), 9.86 (br. s., 1H), |
| | 4-pyrimidinyl}amino)-N- | | | 9.52 (br. s., 1H), 8.35 (s, 1H), 8.05 (s, |
| | methylbenzenesulfonamide | | | 1H), 7.83 (d, J = 8.03 Hz, 1H), |
| | trifluoroacetate | | | 7.42-7.57 (m, 4H), 7.39 (d, J = 7.78 Hz, |
| | | | | 1H), 7.03 (d, J = 8.78 Hz, 2H), 6.13 (s, |
| | | | | 1H), 4.33 (t, J = 4.89 Hz, 2H), 3.51 (q, |
| | | | | J = 5.19 Hz, 2H), 2.86 (d, J = 5.02 Hz, |
| | | | | 6H), 2.44 (d, J = 5.02 Hz, 3H) |
| 226 | N-methyl-3-{[6-({3-[(4-methyl-1- | 1.49^{a} | 468.0 (M + H)⁺ | ¹H NMR (400 MHz, CDCl3) δ ppm |
| | piperazinyl)methyl]phenyl}amino)- | | | 8.28 (s, 1H), 8.04 (br. s., 1H), |
| | 4- | | | 7.77 (br. s., 1H), 7.66 (d, J = 7.78 Hz, 1H), |
| | pyrimidinyl]amino}benzenesulfonamide | | | 7.53 (t, J = 7.91 Hz, 1H), |
| | trifluoroacetate | | | 7.37-7.49 (m, 2H), 7.30-7.37 (m, 1H), 7.14 (d, |
| | | | | J = 7.53 Hz, 1H), 6.40 (br. s., 1H), |
| | | | | 3.95 (br. s., 2H), 2.89-3.45 (m, 8H), |
| | | | | 2.76 (s, 3H), 2.67 (d, J = 5.27 Hz, 3H) |
| 227 | N-methyl-3-[(6-{[4- | 2.34^{a} | 423.9 (M + H)⁺ | ¹H NMR (400 MHz, DMSO-d6) δ ppm |
| | (trifluoromethyl)phenyl]amino}-4- | | | 9.71 (s, 1H), 9.67 (s, 1H), 8.42 (s, |
| | pyrimidinyl)amino]benzenesulfonamide | | | 1H), 8.11 (t, J = 1.88 Hz, 1H), |
| | trifluoroacetate | | | 7.93 (dd, J = 1.51, 8.28 Hz, 1H), 7.86 (d, J = 8.78 Hz, |
| | | | | 2H), 7.65 (d, J = 8.78 Hz, |
| | | | | 2H), 7.53 (t, J = 8.03 Hz, 1H), 7.45 (q, |
| | | | | J = 5.02 Hz, 1H), 7.35 (d, J = 7.78 Hz, |
| | | | | 1H), 6.27 (s, 1H), 2.45 (d, J = 5.02 Hz, |
| | | | | 3H) |
| 228 | N-methyl-3-[(6-{[4-(1- | 2.28^{a} | 398.0 (M + H)⁺ | ¹H NMR (400 MHz, DMSO-d6) δ ppm |
| | methylethyl)phenyl]amino}-4- | | | 9.79 (br. s., 1H), 9.47 (br. s., 1H), |
| | pyrimidinyl)amino]benzenesulfonamide | | | 8.36 (s, 1H), 8.06 (s, 1H), 7.85 (d, J = 8.03 Hz, |
| | trifluoroacetate | | | 1H), 7.54 (t, J = 7.91 Hz, 1H), |
| | | | | 7.46 (q, J = 5.02 Hz, 1H), |
| | | | | 7.35-7.42 (m, 3H), 7.24 (d, J = 8.53 Hz, 2H), |
| | | | | 6.16 (s, 1H), 2.88 (dt, J = 6.90, 13.80 Hz, |
| | | | | 1H), 2.44 (d, J = 5.02 Hz, 3H), |
| | | | | 1.21 (d, J = 6.78 Hz, 6H) |
| 229 | N-methyl-3-{[6-({4-[(1- | 2.11^{a} | 414.0 (M + H)⁺ | ¹H NMR (400 MHz, DMSO-d6) δ ppm |
| | methylethyl)oxy]phenyl}amino)- | | | 9.77 (br. s., 1H), 9.35 (br. s., 1H), |
| | 4- | | | 8.33 (s, 1H), 8.04 (s, 1H), 7.76-7.88 (m, |
| | pyrimidinyl]amino}benzenesulfonamide | | | 1H), 7.53 (t, J = 8.03 Hz, 1H), 7.46 (q, |
| | trifluoroacetate | | | J = 4.94 Hz, 1H), 7.28-7.42 (m, 3H), |
| | | | | 6.94 (d, J = 8.78 Hz, 2H), 6.05 (s, 1H), |
| | | | | 4.58 (dt, J = 6.02, 12.05 Hz, 1H), |
| | | | | 2.44 (d, J = 5.02 Hz, 3H), 1.27 (d, J = 6.02 Hz, |
| | | | | 6H) |
| 230 | 3-{[6-({4- | 2.08^{a} | 421.9 (M + H)⁺ | ¹H NMR (400 MHz, DMSO-d6) δ ppm |
| | [(difluoromethyl)oxy]phenyl}amino)- | | | 9.68 (s, 1H), 9.46 (s, 1H), 8.36 (s, |
| | 4-pyrimidinyl]amino}-N- | | | 1H), 8.08 (s, 1H), 7.84-7.91 (m, 1H), |
| | methylbenzenesulfonamide | | | 7.59 (d, J = 9.03 Hz, 2H), 7.53 (t, J = 8.03 Hz, |
| | trifluoroacetate | | | 1H), 7.45 (q, J = 4.94 Hz, |
| | | | | 1H), 7.31-7.39 (m, 1H), |
| | | | | 7.13-7.20 (m, 3H), 6.16 (s, 1H), 2.44 (d, J = 5.02 Hz, |
| | | | | 3H) |
| 231 | N-methyl-3-[(6-{[4-(2-oxo-1- | 1.84^{a} | 439.0 (M + H)⁺ | ¹H NMR (400 MHz, METHANOL-d4) δ |
| | pyrrolidinyl)phenyl]amino}-4- | | | ppm 8.32-8.37 (m, 1H), |
| | pyrimidinyl)amino]benzenesulfonamide | | | 8.00-8.06 (m, 1H), 7.70-7.77 (m, 2H), |
| | trifluoroacetate | | | 7.58-7.70 (m, 3H), 7.38-7.47 (m, 2H), |
| | | | | 6.16 (s, 1H), 3.92-4.01 (m, 2H), |
| | | | | 2.59-2.68 (m, 2H), 2.53-2.58 (m, 3H), |
| | | | | 2.16-2.28 (m, 2H) |
| 232 | 3-[(6-{[3-chloro-4- | 2.17^{a} | 420.9 (M + H)⁺ | ¹H NMR (400 MHz, DMSO-d6) δ |
| | (methyloxy)phenyl]amino}-4- | | | 9.71 (s, 1H), 9.39 (br. s., 1H), 8.36 (s, 1H), |
| | pyrimidinyl)amino]-N- | | | 8.06 (s, 1H), 7.85-7.90 (m, 1H), |
| | methylbenzenesulfonamide | | | 7.73 (d, J = 2.51 Hz, 1H), 7.53 (t, J = 8.03 Hz, |
| | trifluoroacetate | | | 1H), 7.45 (q, J = 4.85 Hz, 1H), |
| | | | | 7.34-7.42 (m, 2H), 7.15 (d, J = 9.03 Hz, |
| | | | | 1H), 6.09 (s, 1H), 3.84 (s, 3H), |
| | | | | 2.44 (d, J = 5.02 Hz, 3H) |
| 233 | 3-({6-[(4- | 2.12^{a} | 396.1 (M + H)⁺ | ¹H NMR (400 MHz, DMSO-d6) δ |
| | cyclopropylphenyl)amino]-4- | | | 9.61 (s, 1H), 9.25 (s, 1H), 8.32 (s, 1H), |
| | pyrimidinyl}amino)-N- | | | 8.08 (s, 1H), 7.84-7.89 (m, 1H), |
| | methylbenzenesulfonamide | | | 7.47-7.55 (m, 1H), 7.44 (q, J = 4.94 Hz, |
| | trifluoroacetate | | | 1H), 7.31-7.41 (m, 3H), 7.05 (d, J = 8.53 Hz, |
| | | | | 2H), 6.13 (s, 1H), 2.44 (d, J = 5.02 Hz, |
| | | | | 3H), 1.82-1.95 (m, 1H), |
| | | | | 0.88-0.96 (m, 2H), 0.58-0.67 (m, |
| | | | | 2H) |
| 234 | N-methyl-3-[(6-{[4-(1H-pyrazol- | 1.97^{a} | 422.1 (M + H)⁺ | ¹H NMR (400 MHz, DMSO-d6) δ |
| | 1-yl)phenyl]amino}-4- | | | 9.67 (s, 1H), 9.51 (s, 1H), 8.42 (d, J = 2.26 Hz, |
| | pyrimidinyl)amino]benzenesulfonamide | | | 1H), 8.39 (s, 1H), 8.09 (s, 1H), |
| | trifluoroacetate | | | 7.88-7.94 (m, 1H), 7.75-7.81 (m, |
| | | | | 2H), 7.65-7.74 (m, 3H), 7.53 (t, J = 8.03 Hz, |
| | | | | 1H), 7.45 (q, J = 4.94 Hz, |
| | | | | 1H), 7.36 (d, J = 7.78 Hz, 1H), 6.53 (t, |
| | | | | J = 2.01 Hz, 1H), 6.21 (s, 1H), 2.45 (d, |
| | | | | J = 5.02 Hz, 3H) |
| 235 | 3-[(6-{[4-(3,5-dimethyl-1H- | 2.04^{a} | 450.1 (M + H)⁺ | ¹H NMR (400 MHz, DMSO-d6) δ |
| | pyrazol-1-yl)phenyl]amino}-4- | | | 9.72 (s, 1H), 9.59 (s, 1H), 8.40 (s, 1H), |
| | pyrimidinyl)amino]-N- | | | 8.09 (s, 1H), 7.86-7.93 (m, 1H), |
| | methylbenzenesulfonamide | | | 7.69 (d, J = 8.78 Hz, 2H), 7.54 (t, J = 8.03 Hz, |
| | trifluoroacetate | | | 1H), 7.40-7.50 (m, 3H), 7.37 (d, |
| | | | | J = 7.78 Hz, 1H), 6.23 (s, 1H), 6.05 (s, |
| | | | | 1H), 2.45 (d, J = 4.77 Hz, 3H), 2.28 (s, |
| | | | | 3H), 2.18 (s, 3H) |
| 236 | 3-[(6-{[4-chloro-3- | 2.12^{a} | 420.0 (M + H)⁺ | ¹H NMR (400 MHz, DMSO-d6) δ ppm |
| | (methyloxy)phenyl]amino}-4- | | | 9.69 (s, 1H), 9.52 (s, 1H), 8.39 (s, |
| | pyrimidinyl)amino]-N- | | | 1H), 8.05-8.09 (m, 1H), |
| | methylbenzenesulfonamide | | | 7.87-7.94 (m, 1H), 7.53 (t, J = 8.03 Hz, 1H), |
| | trifluoroacetate | | | 7.45 (q, J = 4.94 Hz, 1H), |
| | | | | 7.32-7.40 (m, 3H), 7.24 (dd, J = 2.13, 8.66 Hz, |
| | | | | 1H), 6.21 (s, 1H), 3.85 (s, 3H), |
| | | | | 2.45 (d, J = 4.77 Hz, 3H) |
| 237 | N-methyl-3-[(6-{[4-(2- | 2.26^{a} | 438.0 (M + H)⁺ | ¹H NMR (400 MHz, DMSO-d6) δ ppm |
| | thienyl)phenyl]amino}-4- | | | 9.71 (s, 1H), 9.55 (s, 1H), 8.39 (s, |
| | pyrimidinyl)amino]benzenesulfonamide | | | 1H), 8.09 (s, 1H), 7.86-7.92 (m, 1H), |
| | trifluoroacetate | | | 7.63 (s, 4H), 7.54 (t, J = 8.03 Hz, 1H), |
| | | | | 7.42-7.51 (m, 3H), 7.37 (d, J = 7.78 Hz, |
| | | | | 1H), 7.13 (dd, J = 3.64, 4.89 Hz, |
| | | | | 1H), 6.23 (s, 1H), 2.45 (d, J = 5.02 Hz, |
| | | | | 3H) |
| 238 | N-methyl-3-[(6-{[4-(2-methyl-1H- | 1.59^{a} | 436.1 (M + H)⁺ | ¹H NMR (400 MHz, DMSO-d6) δ ppm |
| | imidazol-1-yl)phenyl]amino}-4- | | | 9.74 (s, 1H), 9.70 (s, 1H), 8.42 (s, |
| | pyrimidinyl)amino]benzenesulfonamide | | | 1H), 8.09-8.13 (m, 1H), |
| | trifluoroacetate | | | 7.85-7.95 (m, 4H), 7.78 (t, J = 1.63 Hz, 1H), |
| | | | | 7.50-7.57 (m, 3H), 7.46 (q, J = 4.68 Hz, |
| | | | | 1H), 7.36 (d, J = 7.53 Hz, 1H), |
| | | | | 6.29 (s, 1H), 2.53-2.56 (m, 3H), |
| | | | | 2.45 (d, J = 4.02 Hz, 3H) |
| 239 | N-methyl-3-[(6-{[4-(1- | 2.30^{a} | 412.1 (M + H)⁺ | ¹H NMR (400 MHz, DMSO-d6) δ ppm |
| | methylpropyl)phenyl]amino}-4- | | | 9.78 (br. s., 1H), 9.44 (br. s., 1H), |
| | pyrimidinyl)amino]benzenesulfonamide | | | 8.36 (s, 1H), 8.06 (s, 1H), 7.85 (d, J = 8.03 Hz, |
| | trifluoroacetate | | | 1H), 7.54 (t, J = 7.91 Hz, 1H), |
| | | | | 7.46 (q, J = 4.77 Hz, 1H), 7.39 (d, J = 8.28 Hz, |
| | | | | 3H), 7.19 (d, J = 8.28 Hz, |
| | | | | 2H), 6.16 (s, 1H), 2.54-2.62 (m, 1H), |
| | | | | 2.44 (d, J = 4.77 Hz, 3H), |
| | | | | 1.50-1.61 (m, 2H), 1.19 (d, J = 6.78 Hz, 3H), |
| | | | | 0.79 (t, J = 7.40 Hz, 3H) |
| 240 | N-methyl-3-{[6-(6- | 1.68^{a} | 407.1 (M + H)⁺ | ¹H NMR (400 MHz, DMSO-d6) δ ppm |
| | quinolinylamino)-4- | | | 9.55-9.74 (m, 2H), 8.75 (br. s., 1H), |
| | pyrimidinyl]amino}benzenesulfonamide | | | 8.45 (br. s., 1H), 8.33 (br. s., 1H), |
| | | | | 8.26 (d, J = 6.02 Hz, 1H), 8.12 (br. s., 1H), |
| | | | | 7.96 (br. s., 2H), 7.88 (br. s., 1H), |
| | | | | 7.41-7.59 (m, 3H), 7.26-7.41 (m, 1H), |
| | | | | 6.32 (br. s., 1H), 2.45 (br. s., 3H) |
| 241 | N-methyl-3-{[6-({4- | 2.54^{a} | 456.0 (M + H)⁺ | ¹H NMR (400 MHz, DMSO-d6) δ ppm |
| | [(trifluoromethyl)thio]phenyl}amino)- | | | 9.72 (s, 1 H), 9.70 (s, 1 H), 8.42 (s, 1 |
| | 4- | | | H), 8.10 (s, 1 H), 7.93 (d, J = 8.03 Hz, 1 |
| | pyrimidinyl]amino}benzenesulfonamide | | | H), 7.80 (d, J = 8.78 Hz, 2 H), 7.64 (d, |
| | trifluoroacetate | | | J = 8.78 Hz, 2 H), 7.53 (t, J = 8.03 Hz, 1 |
| | | | | H), 7.45 (q, J = 4.94 Hz, 1 H), 7.36 (d, |
| | | | | J = 7.78 Hz, 1 H), 6.27 (s, 1 H), 2.45 (d, |
| | | | | J = 5.02 Hz, 3 H) |
| 242 | 3-({6-[(4-bromophenyl)amino]-4- | 2.21 | 434.0 (M + H)⁺ | ¹H NMR (400 MHz, DMSO-d6) δ ppm |
| | pyrimidinyl}amino)-N- | | | 9.61 (s, 1 H), 9.43 (s, 1 H), 8.37 (s, 1 |
| | methylbenzenesulfonamide | | | H), 8.07-8.13 (m, 1 H), 7.91 (d, |
| | trifluoroacetate | | | J = 8.03 Hz, 1 H), 7.56-7.62 (m, 2 H), |
| | | | | 7.52 (t, J = 8.03 Hz, 1 H), 7.48 (d, |
| | | | | J = 8.78 Hz, 2 H), 7.44 (q, J = 5.10 Hz, 1 |
| | | | | H), 7.34 (d, J = 7.78 Hz, 1 H), 6.19 (s, 1 |
| | | | | H), 2.44 (d, J = 5.02 Hz, 3 H) |
| 243 | N-methyl-3-[(6-{[4- | 2.13^{a} | 402.0 (M + H)⁺ | ¹H NMR (400 MHz, DMSO-d6) δ ppm |
| | (methylthio)phenyl]amino}-4- | | | 9.63 (s, 1 H), 9.36 (s, 1 H), 8.35 (s, 1 |
| | pyrimidinyl)amino]benzenesulfonamide | | | H), 8.09 (s, 1 H), 7.85-7.92 (m, 1 H), |
| | trifluoroacetate | | | 7.49-7.55 (m, 3 H), 7.44 (q, J = 5.02 Hz, |
| | | | | 1 H), 7.35 (d, J = 7.78 Hz, 1 H), |
| | | | | 7.27 (d, J = 8.78 Hz, 2 H), 6.16 (s, 1 H), |
| | | | | 2.46 (s, 3 H), 2.44 (d, J = 4.77 Hz, 3 H) |
| 244 | N-methyl-3-{[6-({4- | 2.31^{a} | 440.1 (M + H)⁺ | ¹H NMR (400 MHz, DMSO-d6) δ ppm |
| | [(trifluoromethyl)oxy]phenyl}amino)- | | | 9.65 (s, 1 H), 9.52 (s, 1 H), 8.37 (s, 1 |
| | 4- | | | H), 8.07-8.14 (m, 1 H), 7.90 (d, |
| | pyrimidinyl]amino}benzenesulfonamide | | | J = 7.78 Hz, 1 H), 7.70 (d, J = 9.04 Hz, 2 |
| | trifluoroacetate | | | H), 7.53 (t, J = 7.91 Hz, 1 H), 7.45 (q, |
| | | | | J = 4.94 Hz, 1 H), 7.30-7.38 (m, 3 H), |
| | | | | 6.20 (s, 1 H), 2.45 (d, J = 5.02 Hz, 3 H) |
| 245 | 3-({6-[(4-chlorophenyl)amino]-4- | 2.12^{a} | 432.9 (M + H)⁺ | ¹H NMR (400 MHz, DMSO-d6) δ ppm |
| | pyrimidinyl}amino)-4- | | | 9.73 (br. s., 1H), 9.21 (br. s., 1H), |
| | (dimethylamino)-N- | | | 8.33 (s, 1H), 7.78-7.83 (m, 1H), 7.55 (d, J = 8.81 Hz, |
| | methylbenzenesulfonamide | | | 2H), 7.51 (dd, J = 2.27, |
| | trifluoroacetate | | | 8.56 Hz, 1H), 7.38 (d, J = 8.81 Hz, |
| | | | | 2H), 7.30 (q, J = 4.95 Hz, 1H), |
| | | | | 7.20 (d, J = 8.56 Hz, 1H), 6.01 (s, 1H), |
| | | | | 2.77 (s, 6H), 2.41 (d, J = 5.04 Hz, 3H) |
| 246 | 4-(dimethylamino)-N-methyl-3- | 2.08^{a} | 413.0 (M + H)⁺ | ¹H NMR (400 MHz, DMSO-d6) δ ppm |
| | ({6-[(3-methylphenyl)amino]-4- | | | 9.84 (br. s., 1H), 9.51 (br. s., 1H), |
| | pyrimidinyl}amino)benzenesulfonamide | | | 8.36 (s, 1H), 7.72-7.77 (m, 1H), 7.54 (dd, |
| | trifluoroacetate | | | J = 2.13, 8.66 Hz, 1H), 7.32 (q, J = 5.02 Hz, |
| | | | | 1H), 7.22-7.29 (m, 3H), |
| | | | | 7.20 (d, J = 8.78 Hz, 1H), 6.96 (d, J = 6.27 Hz, |
| | | | | 1H), 5.98 (s, 1H), 2.77 (s, |
| | | | | 6H), 2.40 (d, J = 4.77 Hz, 3H), 2.30 (s, |
| | | | | 3H) |
| 247 | N-methyl-1-(6-{[4- | 2.56^{a} | 451.0 (M + H)⁺ | ¹H NMR (400 MHz, DMSO-d6) δ ppm |
| | (trifluoromethyl)phenyl]amino}-4- | | | 9.85 (s, 1H), 8.83 (s, 1H), 8.53 (s, |
| | pyrimidinyl)-2,3-dihydro-1H- | | | 1H), 7.92 (d, J = 8.53 Hz, 2H), |
| | indole-6-sulfonamide | | | 7.66 (d, J = 8.53 Hz, 2H), 7.39-7.46 (m, |
| | trifluoroacetate | | | 2H), 7.33 (d, J = 7.78 Hz, 1H), 6.14 (s, |
| | | | | 1H), 4.07 (t, J = 8.66 Hz, 2H), 3.31 (t, |
| | | | | J = 8.66 Hz, 2H), 2.42 (d, J = 4.27 Hz, |
| | | | | 3H) |
| 248 | 1-{6-[(4-chlorophenyl)amino]-4- | 2.06^{a} | 415.1 (M + H)⁺ | ¹H NMR (400 MHz, DMSO-d₆) δ ppm |
| | pyrimidinyl}-N-methyl-1H- | | | 2.42 (d, J = 5.02 Hz, 3 H) 7.15 (s, 1 H) |
| | benzimidazole-6-sulfonamide | | | 7.44 (d, J = 8.78 Hz, 2 H) |
| | trifluoroacetate | | | 7.51-7.60 (m, 1 H) 7.72-7.82 (m, 3 H) 8.01 (d, |
| | | | | J = 8.28 Hz, 1 H) 8.76-8.82 (m, 2 H) |
| | | | | 9.17 (s, 1 H) 10.16 (s, 1 H) |
| 249 | 3-({6-[(5-bromo-6-methyl-2- | 2.20^{a} | 546.8 (M + H)⁺ | ¹H NMR (400 MHz, DMSO-d₆) δ ppm |
| | pyridinyl)amino]-4- | | | 2.44 (d, J = 5.02 Hz, 3 H) 2.50 (s, 3H, |
| | pyrimidinyl}amino)-N-methyl-4- | | | obscured by solvent) 4.91 (q, J = 8.78 Hz, |
| | [(2,2,2- | | | 2 H) 7.12 (br. s., 1 H) 7.25 (d, |
| | trifluoroethyl)oxy]benzenesulfonamide | | | J = 8.53 Hz, 1 H) 7.43-7.50 (m, 2 H) |
| | trifluoroacetate | | | 7.65 (dd, J = 8.66, 2.13 Hz, 1 H) |
| | | | | 7.91 (d, J = 8.78 Hz, 1 H) 7.96 (d, J = 2.01 Hz, |
| | | | | 1 H) 8.39 (s, 1 H) 9.50 (br. s., 1 H) |
| | | | | 10.42 (br. s., 1 H) |
| 250 | 3-({6-[(4-chlorophenyl)amino]-4- | 2.26^{a} | 447.9 (M + H)⁺ | ¹H NMR (400 MHz, DMSO-d6) δ ppm |
| | pyrimidinyl}amino)-N-methyl-4- | | | 9.78 (br. s., 1H), 9.12 (br. s., 1H), |
| | [(1- | | | 8.37 (s, 1H), 8.06 (br. s., 1H), 7.56 (d, J = 8.28 Hz, |
| | methylethyl)oxy]benzenesulfonamide | | | 3H), 7.41 (d, J = 8.78 Hz, |
| | trifluoroacetate | | | 2H), 7.34-7.38 (m, 1H), 7.32 (d, J = 8.78 Hz, |
| | | | | 1H), 6.13 (s, 1H), 4.77 (dt, J = 5.83, |
| | | | | 11.92 Hz, 1H), 2.42 (d, J = 4.52 Hz, |
| | | | | 3H), 1.29 (d, J = 6.02 Hz, |
| | | | | 6H) |
| 251 | 3-({6-[(4-chlorophenyl)amino]-4- | 2.09^{a} | 474.9 (M + H)⁺ | ¹H NMR (400 MHz, DMSO-d6) δ ppm |
| | pyrimidinyl}amino)-N-methyl-4- | | | 9.66 (br. s., 1H), 9.03 (br. s., 1H), |
| | (4- | | | 8.34 (s, 1H), 7.89-7.94 (m, 1H), 7.60 (d, J = 9.03 Hz, |
| | morpholinyl)benzenesulfonamide | | | 2H), 7.54 (dd, J = 2.01, |
| | trifluoroacetate | | | 8.53 Hz, 1H), 7.34-7.42 (m, 3H), |
| | | | | 7.27 (d, J = 8.53 Hz, 1H), 6.07 (s, 1H), |
| | | | | 3.60-3.68 (m, 4H), 2.95-3.01 (m, |
| | | | | 4H), 2.43 (d, J = 5.02 Hz, 3H) |
| 252 | 3-({6-[(4-chlorophenyl)amino]-4- | 2.10^{a} | 420.9 (M + H)⁺ | ¹H NMR (400 MHz, DMSO-d6) δ |
| | pyrimidinyl}amino)-N-methyl-4- | | | ppm 9.62 (br. s., 1H), 9.10 (br. s., |
| | (methyloxy)benzenesulfonamide | | | 1H), 8.34 (s, 1H), 8.25 (br. s., 1H), |
| | trifluoroacetate | | | 7.50-7.61 (m, 3H), 7.39 (d, J = 8.78 Hz, |
| | | | | 2H), 7.34 (q, J = 4.85 Hz, 1H), |
| | | | | 7.29 (s, 1H), 6.21 (s, 1H), 3.93 (s, |
| | | | | 3H), 2.42 (d, J = 5.02 Hz, 3H) |
| 253 | 3-({6-[(4-chlorophenyl)amino]-4- | 2.21^{a} | 446.9 (M + H)⁺ | ¹H NMR (400 MHz, DMSO-d6) δ ppm |
| | pyrimidinyl}amino)-4- | | | 9.70 (br. s., 1H), 9.09 (br. s., 1H), |
| | [ethyl(methyl)amino]-N- | | | 8.34 (s, 1H), 7.82 (br. s., 1H), 7.57 (d, J = 8.28 Hz, |
| | methylbenzenesulfonamide | | | 2H), 7.52 (d, J = 8.28 Hz, |
| | trifluoroacetate | | | 1H), 7.38 (d, J = 8.53 Hz, 2H), |
| | | | | 7.32 (d, J = 4.77 Hz, 1H), 7.23 (d, J = 8.53 Hz, |
| | | | | 1H), 5.99 (s, 1H), 2.98-3.13 (m, |
| | | | | 2H), 2.75 (s, 3H), 2.41 (d, J = 4.27 Hz, |
| | | | | 3H), 1.01 (t, J = 6.78 Hz, 3H) |
| 254 | 3-({6-[(4-chlorophenyl)amino]-4- | 1.99^{a} | 405.9 (M + H)⁺ | ¹H NMR (400 MHz, DMSO-d6) δ ppm |
| | pyrimidinyl}amino)-4-hydroxy-N- | | | 9.71 (br. s., 1H), 9.18 (br. s., 1H), |
| | methylbenzenesulfonamide | | | 8.35 (s, 1H), 8.04 (s, 1H), 7.57 (d, J = 9.03 Hz, |
| | trifluoroacetate | | | 2H), 7.43 (dd, J = 2.26, 8.53 Hz, |
| | | | | 1H), 7.39 (d, J = 8.78 Hz, 2H), |
| | | | | 7.26 (q, J = 4.94 Hz, 1H), 7.07 (d, J = 8.53 Hz, |
| | | | | 1H), 6.15 (s, 1H), 2.40 (d, J = 4.77 Hz, |
| | | | | 3H) |
| 255 | 3-({6-[(4-chlorophenyl)amino]-4- | 2.45^{a} | 407.9 (M + H)⁺ | ¹H NMR (400 MHz, DMSO-d6) δ ppm |
| | pyrimidinyl}amino)-4-fluoro-N- | | | 9.53 (s, 1H), 9.39 (s, 1H), |
| | methylbenzenesulfonamide | | | 8.44-8.53 (m, 1H), 8.34 (s, 1H), 7.62 (d, J = 8.78 Hz, |
| | trifluoroacetate | | | 2H), 7.45-7.56 (m, 3H), 7.37 (d, |
| | | | | J = 8.78 Hz, 2H), 6.30 (s, 1 H), 2.45 (d, |
| | | | | J = 4.77 Hz, 3H) |
| 256 | 3-({6-[(4-chlorophenyl)amino]-4- | 2.14^{a} | 435.9 (M + H)⁺ | ¹H NMR (400 MHz, METHANOL-d4) δ |
| | pyrimidinyl}amino)-N-methyl-4- | | | ppm 8.27-8.30 (m, 1H), |
| | (methylthio)benzenesulfonamide | | | 7.81-7.86 (m, 1H), 7.79 (d, J = 2.01 Hz, 1H), |
| | trifluoroacetate | | | 7.56-7.61 (m, 1H), 7.39-7.45 (m, |
| | | | | 4H), 5.85-5.88 (m, 1H), |
| | | | | 2.53-2.59 (m, 6H) |
| 257 | 3-({6-[(4-chlorophenyl)amino]-4- | 2.52^{a} | 474.0 (M + H)⁺ | ¹H NMR (400 MHz, DMSO-d₆) δ ppm |
| | pyrimidinyl}amino)-N-methyl-4- | | | 2.48 (d, J = 5.02 Hz, 3 H) 6.33 (s, 1 H) |
| | [(trifluoromethyl)oxy]benzenesulfonamide | | | 7.37 (d, J = 9.04 Hz, 2 H) |
| | trifluoroacetate | | | 7.52-7.56 (m, 1 H) 7.61 (d, J = 9.03 Hz, 4 H) |
| | | | | 8.32 (s, 1 H) 8.49-8.51 (m, 1 H) |
| | | | | 9.30-9.34 (m, 1 H) 9.49 (br. s, 1 H) |
| 258 | 3-({6-[(4-chlorophenyl)amino]-4- | 1.78^{a} | 527.1 (M + H)⁺ | ¹H NMR (400 MHz, DMSO-d₆) δ ppm |
| | pyrimidinyl}amino)-N-methyl-4- | | | 1.75-1.87 (m, 1 H) 1.88-2.00 (m, 2 |
| | [(2R)-2-(trifluoromethyl)-1- | | | H) 2.12-2.22 (m, 1 H) 2.42 (d, J = 5.02 Hz, |
| | pyrrolidinyl]benzenesulfonamide | | | 3 H) 3.13-3.24 (m, 1 H) |
| | trifluoroacetate | | | 3.56-3.66 (m, 1 H) 4.75-4.88 (m, 1 H) |
| | | | | 5.78 (s, 1 H) 7.35 (d, J = 9.03 Hz, 3 H) |
| | | | | 7.42 (d, J = 8.78 Hz, 1 H) 7.52 (dd, |
| | | | | J = 8.78, 2.26 Hz, 1 H) 7.56 (d, J = 9.03 Hz, |
| | | | | 2 H) 7.69 (d, J = 2.26 Hz, 1 H) |
| | | | | 8.30 (s, 1 H) 9.09 (br. s., 1 H) 9.57 (br. s., 1 |
| | | | | H) |
| 259 | 3-({6-[(4-chlorophenyl)amino]-4- | 2.18^{a} | 495.1 (M + H)⁺ | ¹H NMR (400 MHz, METHANOL-d₄) δ |
| | pyrimidinyl}amino)-4-(3,3- | | | ppm 2.27-2.38 (m, 2 H) 2.40 (s, 3 H) |
| | difluoro-1-pyrrolidinyl)-N- | | | 3.49 (t, J = 7.03 Hz, 2 H) 3.62 (t, |
| | methylbenzenesulfonamide | | | J = 13.05 Hz, 2 H) 5.62 (s, 1 H) 6.98 (d, |
| | trifluoroacetate | | | J = 8.78 Hz, 1 H) 7.26-7.33 (m, 4 H) |
| | | | | 7.55 (d, J = 2.26 Hz, 1 H) 7.61 (dd, |
| | | | | J = 8.78, 2.26 Hz, 1 H) 8.18 (d, J = 0.75 Hz, |
| | | | | 1 H) |
| 260 | N-methyl-3-[(6-{[4-(1,3-oxazol-5- | 1.94^{a} | 422.9 (M + H)⁺ | ¹H NMR (400 MHz, METHANOL-d4) δ |
| | yl)phenyl]amino}-4- | | | ppm 8.39 (s, 1H), 8.29 (s, 1H), |
| | pyrimidinyl)amino]benzenesulfonamide | | | 8.01-8.09 (m, 1H), 7.83 (d, J = 8.53 Hz, |
| | trifluoroacetate | | | 2H), 7.59-7.73 (m, 3H), |
| | | | | 7.51-7.59 (m, 3H), 6.64-7.41 (m, 1H), 6.25 (s, |
| | | | | 1H), 2.57 (s, 3H) |
| 261 | N-methyl-3-({6-[(3- | 2.00^{a} | 455.0 (M + H)⁺ | ¹H NMR (400 MHz, METHANOL-d4) δ |
| | methylphenyl)amino]-4- | | | ppm 8.24 (s, 1H), 7.97 (d, J = 2.01 Hz, |
| | pyrimidinyl}amino)-4-(4- | | | 1H), 7.64 (dd, J = 2.13, 8.41 Hz, |
| | morpholinyl)benzenesulfonamide | | | 1H), 7.23-7.32 (m, 2H), |
| | trifluoroacetate | | | 7.17-7.22 (m, 2H), 7.00 (d, J = 7.28 Hz, 1H), |
| | | | | 6.11 (s, 1 H), 3.73-3.82 (m, 4H), |
| | | | | 2.98-3.05 (m, 4H), 2.51-2.55 (m, 3H), |
| | | | | 2.36 (s, 3H) |
| 262 | N-methyl-4-(methyloxy)-3-[(6- | 2.29^{a} | 454.1 (M + H)⁺ | ¹H NMR (400 MHz, DMSO-d6) δ ppm |
| | {[4- | | | 9.77 (s, 1H), 9.02 (br. s., 1H), 8.37 (s, |
| | (trifluoromethyl)phenyl]amino}-4- | | | 1H), 8.32 (d, J = 1.76 Hz, 1H), |
| | pyrimidinyl)amino]benzenesulfonamide | | | 7.81 (d, J = 8.53 Hz, 2H), 7.66 (d, J = 8.78 Hz, |
| | trifluoroacetate | | | 2H), 7.52 (dd, J = 2.26, 8.53 Hz, |
| | | | | 1H), 7.33 (q, J = 4.94 Hz, 1H), |
| | | | | 7.28 (d, J = 8.78 Hz, 1H), 6.32 (s, 1H), |
| | | | | 3.93 (s, 3H), 2.42 (d, J = 4.77 Hz, 3H) |
| 263 | N-methyl-4-(methylthio)-3-[(6- | 2.33^{a} | 470.1 (M + H)⁺ | ¹H NMR (400 MHz, METHANOL-d4) δ |
| | {[4- | | | ppm 8.32-8.37 (m, 1H), |
| | (trifluoromethyl)phenyl]amino}-4- | | | 7.83-7.87 (m, 1H), 7.82 (d, J = 1.76 Hz, 1H), |
| | pyrimidinyl)amino]benzenesulfonamide | | | 7.66-7.72 (m, 4H), 7.56-7.63 (m, |
| | trifluoroacetate | | | 1H), 6.00 (s, 1H), 2.58 (s, 6H) |
| 264 | 3-({6-[(3-bromo-5- | 2.25^{a} | 480.0 (M + H)⁺ | ¹H NMR (400 MHz, DMSO-d6) δ ppm |
| | methylphenyl)amino]-4- | | | 9.59 (br. s., 1H), 9.14 (br. s., 1H), |
| | pyrimidinyl}amino)-N-methyl-4- | | | 8.36 (s, 1H), 8.23 (br. s., 1H), 7.72 (s, 1H), |
| | (methyloxy)benzenesulfonamide | | | 7.55 (dd, J = 2.13, 8.66 Hz, 1H), |
| | trifluoroacetate | | | 7.34 (q, J = 4.77 Hz, 1H), 7.26-7.31 (m, |
| | | | | 2H), 7.07 (s, 1H), 6.20 (s, 1H), |
| | | | | 3.93 (s, 3H), 2.42 (d, J = 5.02 Hz, 3H), |
| | | | | 2.29 (s, 3H) |
| 265 | 1-{6-[(3-bromo-5- | 2.47^{a} | 476.0 (M + H)⁺ | ¹H NMR (400 MHz, DMSO-d6) δ ppm |
| | methylphenyl)amino]-4- | | | 9.56 (s, 1H), 8.82 (s, 1H), 8.50 (s, |
| | pyrimidinyl}-N-methyl-2,3- | | | 1H), 7.91 (s, 1H), 7.42 (d, J = 7.03 Hz, |
| | dihydro-1H-indole-6- | | | 2H), 7.30-7.39 (m, 2H), 7.01 (s, 1H), |
| | sulfonamide trifluoroacetate | | | 6.06 (s, 1H), 4.05 (t, J = 8.53 Hz, 2H), |
| | | | | 3.30 (t, J = 8.53 Hz, 2H), 2.42 (d, J = 4.77 Hz, |
| | | | | 3H), 2.30 (s, 3H) |
| 266 | N-methyl-3-{[6-({4-[(2,2,2- | 1.81^{a} | 568.1 (M + H)⁺ | ¹H NMR (400 MHz, DMSO-d₆) δ ppm |
| | trifluoroethyl)oxy]phenyl}amino)- | | | 2.44 (d, J = 5.02 Hz, 3 H) 4.12 (q, |
| | 4-pyrimidinyl]amino}-4-[(2,2,2- | | | J = 10.12 Hz, 3 H) 4.73 (q, J = 9.03 Hz, |
| | trifluoroethyl)thio]benzenesulfonamide | | | 2 H) 5.87 (s, 1 H) 7.05 (d, J = 8.78 Hz, |
| | trifluoroacetate | | | 2 H) 7.43 (d, J = 9.03 Hz, 2 H) 7.53 (d, |
| | | | | J = 5.02 Hz, 1 H) 7.61 (s, 1 H) 7.76 (d, |
| | | | | J = 2.01 Hz, 1 H) 7.84 (d, J = 8.53 Hz, 1 |
| | | | | H) 8.23 (s, 1 H) 9.19-9.30 (m, 1 H) |
| | | | | 9.40 (none, 1 H) |
| 267 | 3-({6-[(3,4- | 2.47^{a} | 475.9 (M + H)⁺ | ¹H NMR (400 MHz, DMSO-d₆) δ ppm |
| | difluorophenyl)amino]-4- | | | 2.48 (d, J = 5.02 Hz, 3 H) 6.32 (s, 1 H) |
| | pyrimidinyl}amino)-N-methyl-4- | | | 7.22-7.31 (m, 1 H) 7.39 (d, J = 10.54 Hz, |
| | [(trifluoromethyl)oxy]benzenesulfonamide | | | 1 H) 7.55 (dd, J = 8.78, 2.26 Hz, 1 |
| | trifluoroacetate | | | H) 7.58-7.68 (m, 2 H) 7.78-7.90 (m, |
| | | | | 1 H) 8.34 (s, 1 H) 8.49 (d, J = 2.26 Hz, |
| | | | | 1 H) 9.36 (s, 1 H) 9.56 (s, 1 H) |
| 268 | N-methyl-3-{[6-(4- | 1.71^{a} | 356.9 (M + H)⁺ | ¹H NMR (400 MHz, DMSO-d6) δ ppm |
| | pyridinylamino)-4- | | | 11.02 (s, 1H), 10.03 (s, 1H), 8.61 (s, |
| | pyrimidinyl]amino}benzenesulfonamide | | | 1H), 8.57 (d, J = 7.03 Hz, 2H), |
| | trifluoroacetate | | | 8.11-8.18 (m, 3H), 7.93-7.99 (m, 1H), |
| | | | | 7.58 (t, J = 8.03 Hz, 1H), 7.49 (q, J = 4.94 Hz, |
| | | | | 1H), 7.42 (d, J = 7.78 Hz, |
| | | | | 1H), 6.48 (s, 1H), 2.42-2.49 (m, 3H) |
| 269 | N-methyl-3-{[6-(3- | 1.59^{a} | 356.9 (M + H)⁺ | ¹H NMR (400 MHz, DMSO-d6) δ ppm |
| | pyridinylamino)-4- | | | 9.63 (s, 1H), 9.48 (s, 1H), 8.75 (d, J = 2.51 Hz, |
| | pyrimidinyl]amino}benzenesulfonamide | | | 1H), 8.37 (s, 1H), 8.19 (dd, J = 1.13, |
| | | | | 4.64 Hz, 1H), 8.08-8.14 (m, |
| | | | | 2H), 7.89-7.95 (m, 1H), 7.52 (t, J = 8.03 Hz, |
| | | | | 1H), 7.45 (q, J = 5.02 Hz, |
| | | | | 1H), 7.29-7.37 (m, 2H), 6.23 (s, 1H), |
| | | | | 2.45 (d, J = 5.02 Hz, 3H) |
| 270 | N-methyl-3-({6-[(5-methyl-3- | 1.67^{a} | 370.9 (M + H)⁺ | ¹H NMR (400 MHz, DMSO-d6) δ ppm |
| | pyridinyl)amino]-4- | | | 9.61 (s, 1H), 9.40 (s, 1H), |
| | pyrimidinyl}amino)benzenesulfonamide | | | 8.51-8.58 (m, 1H), 8.37 (s, 1H), 8.08-8.15 (m, |
| | | | | 1H), 8.04 (s, 1H), 7.89-8.00 (m, 2H), |
| | | | | 7.52 (t, J = 7.91 Hz, 1H), |
| | | | | 7.41-7.48 (m, 1H), 7.34 (d, J = 6.78 Hz, 1H), |
| | | | | 6.21 (s, 1H), 2.44 (d, J = 5.02 Hz, 3H), |
| | | | | 2.30 (s, 3H) |
| 271 | N-methyl-3-{[6-(2- | 1.77^{a} | 356.9 (M + H)⁺ | ¹H NMR (400 MHz, DMSO-d6) δ ppm |
| | pyridinylamino)-4- | | | 9.94 (br. s., 1H), 9.80 (s, 1H), 8.39 (s, |
| | pyrimidinyl]amino}benzenesulfonamide | | | 1H), 8.30 (d, J = 3.76 Hz, 1H), 8.23 (s, |
| | | | | 1H), 7.94 (d, J = 7.78 Hz, 1H), 7.71 (t, |
| | | | | J = 7.03 Hz, 1H), 7.40-7.57 (m, 4H), |
| | | | | 7.34 (d, J = 7.53 Hz, 1H), |
| | | | | 6.91-7.01 (m, 1H), 2.46 (d, J = 5.02 Hz, 3H) |
| 272 | N-methyl-5-{[6-({3- | 1.89^{a} | 449.9 (M + H)⁺ | ¹H NMR (400 MHz, DMSO-d6) δ ppm |
| | [(methylamino)sulfonyl]phenyl}amino)- | | | 9.89 (br. s., 1H), 9.73 (br. s., 1H), |
| | 4-pyrimidinyl]amino}-3- | | | 8.99 (br. s., 1H), 8.65 (br. s., 1H), 8.45 (s, |
| | pyridinesulfonamide | | | 1H), 8.49 (s, 1H), 8.09 (br. s., 1H), |
| | | | | 7.96 (br. s., 1H), 7.74 (br. s., 1H), |
| | | | | 7.54 (br. s., 1H), 7.46 (br. s., 1H), 7.38 (br. |
| | | | | s., 1H), 6.27 (br. s., 1H), 3.35 (br. s., |
| | | | | 3H), 2.46 (br. s., 3H) |
| 273 | 3-({6-[(5-chloro-2- | 1.97^{a} | 390.9 (M + H)⁺ | ¹H NMR (400 MHz, DMSO-d6) δ ppm |
| | pyridinyl)amino]-4- | | | 10.32 (br. s., 1H), 9.97 (br. s., 1H), |
| | pyrimidinyl}amino)-N- | | | 8.44 (s, 1H), 8.31 (d, J = 2.51 Hz, 1H), |
| | methylbenzenesulfonamide | | | 8.18 (s, 1H), 7.88-7.94 (m, 1H), |
| | trifluoroacetate | | | 7.85 (dd, J = 2.64, 8.91 Hz, 1H), |
| | | | | 7.51-7.59 (m, 2H), 7.46 (q, J = 4.85 Hz, |
| | | | | 1H), 7.38 (d, J = 7.78 Hz, 1H), 7.24 (s, |
| | | | | 1H), 2.45 (d, J = 5.02 Hz, 3H) |
| 274 | N-methyl-3-{[6-(1,3-thiazol-2- | 5.50^{b} | 363.0 (M + H)⁺ | ¹H NMR (400 MHz, DMSO-d6) δ ppm |
| | ylamino)-4- | | | 11.42 (s, 1H), 9.83 (s, 1H), 8.50 (s, |
| | pyrimidinyl]amino}benzenesulfonamide | | | 1H), 8.14 (t, J = 1.76 Hz, 1H), |
| | trifluoroacetate | | | 7.87-7.99 (m, 1H), 7.53 (t, J = 8.03 Hz, |
| | | | | 1H), 7.44 (q, J = 5.02 Hz, 1H), |
| | | | | 7.41 (d, J = 3.51 Hz, 1H), 7.36 (d, J = 7.78 Hz, |
| | | | | 1H), 7.11 (d, J = 3.76 Hz, 1H), |
| | | | | 6.53 (s, 1H), 2.45 (d, J = 5.02 Hz, 3H) |
| 275 | N-methyl-3-[(6-{[5- | 2.11^{a} | 424.9 (M + H)⁺ | ¹H NMR (400 MHz, DMSO-d6) δ ppm |
| | (trifluoromethyl)-2- | | | 10.57 (br. s., 1H), 9.98 (s, 1H), |
| | pyridinyl]amino}-4- | | | 8.62 (s, 1H), 8.48 (s, 1H), 8.20 (s, 1H), |
| | pyrimidinyl)amino]benzenesulfonamide | | | 8.05-8.12 (m, 1H), 7.93 (d, J = 8.03 Hz, |
| | trifluoroacetate | | | 1H), 7.72 (d, J = 8.78 Hz, 1H), |
| | | | | 7.55 (t, J = 8.03 Hz, 1H), 7.46 (q, J = 4.60 Hz, |
| | | | | 1H), 7.35-7.42 (m, 2H), |
| | | | | 2.46 (d, J = 4.77 Hz, 3H) |
| 276 | N-methyl-3-({6-[(5-methyl-1,3- | 5.79^{b} | 377.0 (M + H)⁺ | ¹H NMR (400 MHz, DMSO-d6) δ ppm |
| | thiazol-2-yl)amino]-4- | | | 11.29 (br. s., 1H), 9.84 (s, 1H), |
| | pyrimidinyl}amino)benzenesulfonamide | | | 8.46 (s, 1H), 8.14 (s, 1H), 7.88-7.94 (m, |
| | | | | 1H), 7.53 (t, J = 7.91 Hz, 1H), 7.45 (q, |
| | | | | J = 4.94 Hz, 1H), 7.36 (d, J = 7.78 Hz, |
| | | | | 1H), 7.08 (s, 1H), 6.51 (s, 1H), |
| | | | | 2.45 (d, J = 5.02 Hz, 3H), 2.34 (s, 3H) |
| 277 | N-methyl-3-{[6-(1,3,4-thiadiazol- | 5.63^{b} | 364.0 (M + H)⁺ | ¹H NMR (400 MHz, DMSO-d6) δ ppm |
| | 2-ylamino)-4- | | | 11.84 (s, 1H), 9.93 (s, 1H), 9.07 (s, |
| | pyrimidinyl]amino}benzenesulfonamide | | | 1H), 8.52 (s, 1H), 8.11-8.17 (m, 1H), |
| | | | | 7.89-7.96 (m, 1H), 7.54 (t, J = 7.91 Hz, |
| | | | | 1H), 7.46 (q, J = 4.94 Hz, 1H), |
| | | | | 7.37 (d, J = 7.78 Hz, 1H), 6.53 (s, 1H), |
| | | | | 2.45 (d, J = 5.02 Hz, 3H) |
| 278 | 3-{[6-(3-isoquinolinylamino)-4- | 2.03^{a} | 407.1 (M + H)⁺ | ¹H NMR (400 MHz, DMSO-d6) δ ppm |
| | pyrimidinyl]amino}-N- | | | 10.07 (s, 1 H), 9.74 (s, 1 H), 9.14 (s, 1 |
| | methylbenzenesulfonamide | | | H), 8.47 (s, 1 H), 8.19 (s, 1 H), |
| | | | | 8.22 (s, 1 H), 8.04 (d, J = 8.3 Hz, 1 H), |
| | | | | 7.95 (d, J = 8.3 Hz, 1 H), 7.83 (d, J = 8.5 Hz, |
| | | | | 1 H), 7.67 (t, J = 7.7 Hz, 1 H), |
| | | | | 7.52 (t, J = 7.9 Hz, 1 H), |
| | | | | 7.49-7.41 (m, 2 H), 7.34 (d, J = 7.8 Hz, 1 H), |
| | | | | 6.96 (s, 1 H), 2.46 (br. s., 3 H) |
| 279 | N-methyl-3-{[6-(2- | 2.02^{a} | 407.1 (M + H)⁺ | ¹H NMR (400 MHz, DMSO-d6) δ ppm |
| | quinolinylamino)-4- | | | 10.27 (s, 1 H), 9.84 (s, 1 H), 8.44 (s, 1 |
| | pyrimidinyl]amino}benzenesulfonamide | | | H), 8.25-8.18 (m, 3 H), |
| | | | | 8.10-8.04 (m, 1 H), 7.90 (d, J = 8.5 Hz, 1 H), |
| | | | | 7.84 (d, J = 7.3 Hz, 1 H), 7.71 (td, J = 1.4, |
| | | | | 7.6 Hz, 1 H), 7.57 (t, J = 8.0 Hz, 1 |
| | | | | H), 7.50-7.42 (m, 3 H), |
| | | | | 7.42-7.36 (m, 1 H), 2.47 (d, J = 5.0 Hz, 3 H) |
| 280 | N-methyl-3-{[6-(1,3-oxazol-2- | 4.79^{b} | 347.1 (M + H)⁺ | ¹H NMR (400 MHz, DMSO-d6) δ ppm |
| | ylamino)-4- | | | 11.04 (br. s., 1 H), 9.98 (br. s., 1 H), |
| | pyrimidinyl]amino}benzenesulfonamide | | | 8.41 (s, 1 H), 8.26 (s, 1 H), 7.93 (d, J = 8.8 Hz, |
| | trifluoroacetate | | | 1 H), 7.80 (s, 1 H), |
| | | | | 7.56-7.49 (m, 2 H), 7.44 (q, J = 4.8 Hz, 1 |
| | | | | H), 7.36 (d, J = 7.8 Hz, 1 H), 7.12 (s, 1 |
| | | | | H), 2.45 (d, J = 5.0 Hz, 3 H) |
| 281 | N-methyl-3-[(6-{[4- | 1.28^{d} | 431.1 (M + H)⁺ | ¹H NMR (400 MHz, DMSO-d6) δ ppm |
| | (trifluoromethyl)-1,3-thiazol-2- | | | 11.89 (s, 1 H), 9.88 (s, 1 H), 8.51 (s, 1 |
| | yl]amino}-4- | | | H), 8.10 (s, 1 H), 7.88 (m, 1 H), |
| | pyrimidinyl)amino]benzenesulfonamide | | | 7.77 (s, 1 H), 7.49 (t, J = 7.94 Hz, 1 H), |
| | | | | 7.38-7.45 (m, 1 H), 7.33 (d, J = 7.28 Hz, 1 |
| | | | | H), 6.38 (s, 1 H), 2.41 (d, J = 4.85 Hz, 3 |
| | | | | H) |
| 282 | methyl (2-{[6-({3- | 1.04^{d} | 435.2 (M + H)⁺ | ¹H NMR (400 MHz, DMSO-d6) δ ppm |
| | [(methylamino)sulfonyl]phenyl}amino)- | | | 11.52 (br. s., 1 H), 9.81 (s, 1 H), |
| | 4-pyrimidinyl]amino}-1,3- | | | 8.47 (s, 1 H), 8.07-8.14 (m, 1 H), 7.89 (dt, |
| | thiazol-4-yl)acetate | | | J = 8.21, 1.19 Hz, 1 H), 7.50 (t, J = 7.94 Hz, |
| | trifluoroacetate | | | 1 H), 7.42 (q, J = 4.85 Hz, 1 H), |
| | | | | 7.33 (dd, J = 8.05, 1.43 Hz, 1 H), |
| | | | | 6.85 (s, 1 H), 6.38 (s, 1 H), 3.66 (s, 2 H), |
| | | | | 3.60 (s, 3H), |
| | | | | 2.43 (d, J = 5.07 Hz, 3 H) |
| 283 | N-methyl-3-[(6-{[4-(1- | 1.12^{d} | 405.1 (M + H)⁺ | ¹H NMR (400 MHz, DMSO-d6) δ ppm |
| | methylethyl)-1,3-thiazol-2- | | | 11.41 (br. s., 1 H), 9.79 (s, 1 H), |
| | yl]amino}-4- | | | 8.45 (s, 1 H), 8.10 (t, J = 1.98 Hz, 1 H), |
| | pyrimidinyl)amino]benzenesulfonamide | | | 7.90 (dd, J = 2.21, 0.88 Hz, 1 H), 7.50 (t, |
| | trifluoroacetate | | | J = 7.94 Hz, 1 H), 7.41 (q, J = 5.29 Hz, 1 |
| | | | | H), 7.29-7.37 (m, 1 H), 6.60 (d, |
| | | | | J = 1.10 Hz, 1 H), 6.43 (s, 1 H), |
| | | | | 2.83-2.90 (m, 1 H), 2.43 (d, J = 5.07 Hz, 3 |
| | | | | H), 1.21 (d, J = 6.84 Hz, 6 H) |
| 284 | N-methyl-3-({6-[(4-methyl-1,3- | 0.88^{d} | 361.2 (M + H)⁺ | ¹H NMR (400 MHz, DMSO-d6) δ ppm |
| | oxazol-2-yl)amino]-4- | | | 10.81 (br. s, 1H), 9.93 (s, 1 H), |
| | pyrimidinyl}amino)benzenesulfonamide | | | 8.37 (s, 1 H), 8.24 (br. s., 1 H), 7.93 (m, 1 |
| | | | | H), 7.30-7.55 (m, 5 H), 2.43 (m, 3H), |
| | | | | 2.08 (s, 3 H) |
| 285 | N-methyl-4-(methyloxy)-3-{[6-(2- | 1.99^{a} | 387.1 (M + H)⁺ | ¹H NMR (400 MHz, DMSO-d₆) δ ppm |
| | pyridinylamino)-4- | | | 2.43 (d, J = 5.02 Hz, 3 H) 3.93 (s, 3 H) |
| | pyrimidinyl]amino}benzenesulfonamide | | | 6.98 (br. s., 1 H) 7.12 (t, J = 6.15 Hz, 1 |
| | trifluoroacetate | | | H) 7.31 (d, J = 8.78 Hz, 1 H) |
| | | | | 7.34-7.40 (m, 2 H) 7.59 (dd, J = 8.53, 2.01 Hz, |
| | | | | 1 H) 7.88 (t, J = 7.78 Hz, 1 H) |
| | | | | 8.19 (br. s., 1 H) 8.34 (dd, J = 5.02, 1.26 Hz, |
| | | | | 1 H) 8.46 (s, 1 H) 9.53 (br. s., 1 H) |
| | | | | 10.91 (br. s., 1 H) |
| 286 | 3-({6-[(5-chloro-2- | 2.11^{a} | 421.0 (M + H)⁺ | ¹H NMR (400 MHz, DMSO-d₆) δ ppm |
| | pyridinyl)amino]-4- | | | 2.43 (d, J = 5.02 Hz, 3 H) 3.93 (s, 3 H) |
| | pyrimidinyl}amino)-N-methyl-4- | | | 7.18 (br. s., 1 H) 7.27-7.30 (m, 1 H) |
| | (methyloxy)benzenesulfonamide | | | 7.30-7.34 (m, 1 H) 7.49-7.56 (m, 2 |
| | trifluoroacetate | | | H) 7.85 (dd, J = 9.03, 2.76 Hz, 1 H) |
| | | | | 8.26-8.28 (m, 1 H) 8.29-8.31 (m, 1 |
| | | | | H) 8.38 (s, 1 H) 9.15 (br. s., 1 H) |
| | | | | 10.31 (br. s., 1 H) |
| 287 | 3-({6-[(5-chloro-2- | 2.34^{a} | 489.0 (M + H)⁺ | ¹H NMR (400 MHz, DMSO-d₆) δ ppm |
| | pyridinyl)amino]-4- | | | 2.44 (d, J = 5.02 Hz, 3 H) 4.92 (q, |
| | pyrimidinyl}amino)-N-methyl-4- | | | J = 8.78 Hz, 2 H) 7.05 (br. s., 1 H) |
| | [(2,2,2- | | | 7.42-7.47 (m, 2 H) 7.49 (d, J = 8.78 Hz, 1 |
| | trifluoroethyl)oxy]benzenesulfonamide | | | H) 7.62 (dd, J = 8.66, 2.13 Hz, 1 H) |
| | trifluoroacetate | | | 7.87 (dd, J = 8.91, 2.64 Hz, 1 H) |
| | | | | 8.01 (d, J = 2.01 Hz, 1 H) 8.31 (d, J = 2.51 Hz, |
| | | | | 1 H) 8.38 (s, 1 H) 9.47 (br. s., 1 H) |
| | | | | 10.49 (br. s., 1 H) |
| 288 | N-methyl-3-{[6-(2- | 2.08^{a} | 455.1 (M + H)⁺ | ¹H NMR (400 MHz, DMSO-d₆) δ ppm |
| | pyridinylamino)-4- | | | 2.44 (d, J = 5.02 Hz, 3 H) 4.93 (d, |
| | pyrimidinyl]amino}-4-[(2,2,2- | | | J = 8.78 Hz, 2 H) 7.13-7.19 (m, 1 H) |
| | trifluoroethyl)oxy]benzenesulfonamide | | | 7.29-7.34 (m, 1 H) 7.47 (d, J = 8.78 Hz, |
| | trifluoroacetate | | | 2 H) 7.63-7.68 (m, 1 H) |
| | | | | 7.88-7.94 (m, 1 H) 7.94-7.99 (m, 1 H) |
| | | | | 8.32-8.37 (m, 1 H) 8.46 (s, 1 H) |
| | | | | 9.67-9.76 (m, 1 H) 10.84-10.98 (m, 1 H) |
| 289 | 3-({6-[(5-chloro-2- | 2.13^{a} | 437.0 (M + H)⁺ | ¹H NMR (400 MHz, DMSO-d₆) δ ppm |
| | pyridinyl)amino]-4- | | | 2.44 (d, J = 4.77 Hz, 3 H) 2.50 (s, 3H, |
| | pyrimidinyl}amino)-N-methyl-4- | | | obscured by solvent) 6.76-6.86 (m, 1 |
| | (methylthio)benzenesulfonamide | | | H) 7.49 (d, J = 3.76 Hz, 2 H) 7.55 (d, |
| | trifluoroacetate | | | J = 8.78 Hz, 1 H) 7.69 (br. s., 2 H) |
| | | | | 7.88 (dd, J = 8.78, 2.01 Hz, 1 H) 8.28 (d, |
| | | | | J = 1.76 Hz, 1 H) 8.36 (s, 1 H) 9.60 (br. |
| | | | | s., 1 H) 10.57 (br. s., 1 H) |
| 290 | 1-{6-[(5-chloro-2- | 2.18^{a} | 417.0 (M + H)⁺ | ¹H NMR (400 MHz, DMSO-d₆) δ ppm |
| | pyridinyl)amino]-4-pyrimidinyl}- | | | 2.43 (d, J = 4.77 Hz, 3 H) 3.31 (t, |
| | N-methyl-2,3-dihydro-1H-indole- | | | J = 8.53 Hz, 2 H) 4.11 (t, J = 8.66 Hz, 2 |
| | 6-sulfonamide trifluoroacetate | | | H) 7.16 (s, 1 H) 7.36 (dd, J = 7.78, 1.51 Hz, |
| | | | | 1 H) 7.40-7.48 (m, 2 H) 7.69 (d, |
| | | | | J = 8.78 Hz, 1 H) 7.86 (dd, J = 8.91, |
| | | | | 2.64 Hz, 1 H) 8.37 (d, J = 2.51 Hz, 1 H) |
| | | | | 8.53 (s, 1 H) 8.78 (s, 1 H) 10.36 (br. |
| | | | | s., 1 H) |
| 291 | N-methyl-4-[(2,2,2- | 2.23^{a} | 523.0 (M + H)⁺ | ¹H NMR (500 MHz, DMSO-d₆) δ ppm |
| | trifluoroethyl)oxy]-3-[(6-{[5- | | | 2.44 (d, J = 5.13 Hz, 3 H) 4.90 (q, |
| | (trifluoromethyl)-2- | | | J = 8.79 Hz, 2 H) 7.23 (br. s., 1 H) |
| | pyridinyl]amino}-4- | | | 7.37-7.45 (m, 2 H) 7.57 (dd, J = 8.55, 1.95 Hz, |
| | pyrimidinyl)amino]benzenesulfonamide | | | 1 H) 7.72 (d, J = 8.79 Hz, 1 H) |
| | trifluoroacetate | | | 8.04-8.09 (m, 2 H) 8.36 (s, 1 H) 8.59 (s, 1 |
| | | | | H) 9.12 (br. s., 1 H) 10.45 (br. s., 1 H) |
| 292 | N-methyl-3-{[6-(4- | 1.82^{a} | 455.0 (M + H)⁺ | ¹H NMR (400 MHz, DMSO-d₆) δ ppm |
| | pyridinylamino)-4- | | | 2.44 (d, J = 5.02 Hz, 3 H) 4.92 (q, |
| | pyrimidinyl]amino}-4-[(2,2,2- | | | J = 8.78 Hz, 2 H) 6.24 (s, 1 H) |
| | trifluoroethyl)oxy]benzenesulfonamide | | | 7.37-7.45 (m, 2 H) 7.55 (dd, J = 8.78, 2.26 Hz, |
| | trifluoroacetate | | | 1 H) 7.60-7.66 (m, 2 H) 8.15 (d, |
| | | | | J = 2.26 Hz, 1 H) 8.32-8.36 (m, 3 H) |
| | | | | 8.89 (s, 1 H) 9.66 (s, 1 H) |
| 293 | 3-({6-[(3-fluoro-2- | 1.95^{a} | 473.0 (M + H)⁺ | ¹H NMR (400 MHz, DMSO-d₆) δ ppm |
| | pyridinyl)amino]-4- | | | 2.44 (d, J = 5.02 Hz, 3 H) 4.93 (q, |
| | pyrimidinyl}amino)-N-methyl-4- | | | J = 8.70 Hz, 2 H) 7.07 (br. s., 1 H) |
| | [(2,2,2- | | | 7.16-7.23 (m, 1 H) 7.43-7.50 (m, 2 H) |
| | trifluoroethyl)oxy]benzenesulfonamide | | | 7.64 (dd, J = 8.78, 2.26 Hz, 1 H) |
| | trifluoroacetate | | | 7.77-7.86 (m, 1 H) 8.01 (d, J = 2.01 Hz, 1 H) |
| | | | | 8.19 (d, J = 4.77 Hz, 1 H) 8.42 (s, 1 H) |
| | | | | 9.67 (br. s., 1 H) 10.14 (br. s., 1 H) |
| 294 | 3-({6-[(5-cyano-2- | 2.14^{a} | 480.1 (M + H)⁺ | ¹H NMR (400 MHz, DMSO-d₆) δ ppm |
| | pyridinyl)amino]-4- | | | 2.44 (d, J = 5.02 Hz, 3 H) 4.91 (q, |
| | pyrimidinyl}amino)-N-methyl-4- | | | J = 8.78 Hz, 2 H) 7.27 (s, 1 H) |
| | [(2,2,2- | | | 7.39-7.44 (m, 2 H) 7.56 (dd, J = 8.66, 2.38 Hz, |
| | trifluoroethyl)oxy]benzenesulfonamide | | | 1 H) 7.70 (d, J = 8.78 Hz, 1 H) |
| | trifluoroacetate | | | 8.08 (d, J = 2.26 Hz, 1 H) 8.11 (dd, J = 8.91, |
| | | | | 2.38 Hz, 1 H) 8.34 (s, 1 H) 8.69 (d, |
| | | | | J = 1.76 Hz, 1 H) 9.14 (s, 1 H) 10.48 (s, |
| | | | | 1 H) |
| 295 | N-methyl-3-{[6-(4- | 1.83^{a} | 456.1 (M + H)⁺ | ¹H NMR (400 MHz, DMSO-d₆) δ ppm |
| | pyrimidinylamino)-4- | | | 2.44 (d, J = 4.02 Hz, 3 H) 4.91 (q, |
| | pyrimidinyl]amino}-4-[(2,2,2- | | | J = 8.62 Hz, 2 H) 7.30 (s, 1 H) 7.41 (d, |
| | trifluoroethyl)oxy]benzenesulfonamide | | | J = 8.53 Hz, 2 H) 7.55 (dd, J = 8.66, |
| | | | | 1.88 Hz, 1 H) 7.59 (d, J = 6.02 Hz, 1 H) |
| | | | | 8.10 (d, 1 H) 8.34 (s, 1 H) 8.47 (d, |
| | | | | J = 6.02 Hz, 1 H) 8.76 (s, 1 H) 9.08 (s, |
| | | | | 1 H) 10.30 (s, 1 H) |
| 296 | 3-({6-[(5-chloro-3-fluoro-2- | 2.26^{a} | 507.0 (M + H)⁺ | ¹H NMR (400 MHz, DMSO-d₆) δ ppm |
| | pyridinyl)amino]-4- | | | 2.43 (s, 3 H) 4.91 (q, J = 8.78 Hz, 2 H) |
| | pyrimidinyl}amino)-N-methyl-4- | | | 7.24 (d, J = 0.75 Hz, 1 H) |
| | [(2,2,2- | | | 7.38-7.42 (m, 2 H) 7.53 (dd, J = 8.66, 2.38 Hz, 1 |
| | trifluoroethyl)oxy]benzenesulfonamide | | | H) 8.04 (dd, J = 10.29, 2.26 Hz, 1 H) |
| | | | | 8.17 (d, J = 2.26 Hz, 1 H) 8.22 (d, |
| | | | | J = 2.26 Hz, 1 H) 8.27 (d, J = 0.75 Hz, 1 |
| | | | | H) 8.92 (br. s., 1 H) 9.54 (br. s., 1 H) |
| 297 | N-methyl-4-[(2,2,2- | 2.53^{a} | 523.0 (M + H)⁺ | ¹H NMR (400 MHz, DMSO-d₆) δ ppm |
| | trifluoroethyl)oxy]-3-[(6-{[6- | | | 2.50 (d, J = 4.77 Hz, 3 H) 4.98 (q, |
| | (trifluoromethyl)-3- | | | J = 8.78 Hz, 2 H) 6.30 (s, 1 H) |
| | pyridinyl]amino}-4- | | | 7.45-7.50 (m, 2 H) 7.61 (dd, J = 8.66, 2.13 Hz, |
| | pyrimidinyl)amino]benzenesulfonamide | | | 1 H) 7.87 (d, J = 8.53 Hz, 1 H) |
| | | | | 8.20 (d, J = 2.26 Hz, 1 H) 8.39-8.41 (m, 1 |
| | | | | H) 8.52 (dd, J = 8.53, 2.26 Hz, 1 H) |
| | | | | 8.92 (d, J = 2.26 Hz, 1 H) 8.98 (s, 1 H) |
| | | | | 9.91 (s, 1 H) |
| 298 | 3-({6-[(5-chloro-4-methyl-2- | 2.16^{a} | 503.0 (M + H)⁺ | ¹H NMR (400 MHz, DMSO-d₆) δ ppm |
| | pyridinyl)amino]-4- | | | 2.32 (s, 3 H) 2.44 (d, J = 4.27 Hz, 3 H) |
| | pyrimidinyl}amino)-N-methyl-4- | | | 4.90 (q, J = 8.78 Hz, 2 H) 7.19 (s, 1 H) |
| | [(2,2,2- | | | 7.37-7.44 (m, 2 H) 7.49-7.58 (m, 2 |
| | trifluoroethyl)oxy]benzenesulfonamide | | | H) 8.13 (d, J = 2.01 Hz, 1 H) 8.21 (s, 1 |
| | trifluoroacetate | | | H) 8.28 (s, 1 H) 8.86 (s, 1 H) 9.91 (s, |
| | | | | 1 H) |
| 299 | 3-({6-[(4,5-dichloro-2- | 2.31^{a} | 522.8 (M + H)⁺ | ¹H NMR (400 MHz, DMSO-d₆) δ ppm |
| | pyridinyl)amino]-4- | | | 2.44 (d, J = 3.26 Hz, 3 H) 4.90 (q, |
| | pyrimidinyl}amino)-N-methyl-4- | | | J = 8.95 Hz, 2 H) 7.00 (s, 1 H) |
| | [(2,2,2- | | | 7.38-7.44 (m, 2 H) 7.55 (dd, J = 8.66, 2.13 Hz, |
| | trifluoroethyl)oxy]benzenesulfonamide | | | 1 H) 8.06 (s, 1 H) 8.09 (d, J = 2.26 Hz, |
| | trifluoroacetate | | | 1 H) 8.33 (s, 1 H) 8.42 (s, 1 H) |
| | | | | 8.97 (s, 1 H) 10.18 (s, 1 H) |
| 300 | 3-({6-[(5-chloro-6-methyl-2- | 1.69^{a} | 503.1 (M + H)⁺ | ¹H NMR (400 MHz, DMSO-d₆) δ ppm |
| | pyridinyl)amino]-4- | | | 2.44 (d, J = 5.02 Hz, 3 H) 2.47 (s, 3 H) |
| | pyrimidinyl}amino)-N-methyl-4- | | | 4.91 (q, J = 8.78 Hz, 2 H) 7.12 (br. s., 1 |
| | [(2,2,2- | | | H) 7.33 (d, J = 8.53 Hz, 1 H) |
| | trifluoroethyl)oxy]benzenesulfonamide | | | 7.42-7.50 (m, 2 H) 7.65 (dd, J = 8.78, 2.26 Hz, |
| | trifluoroacetate | | | 1 H) 7.78 (d, J = 8.78 Hz, 1 H) |
| | | | | 7.97 (d, J = 2.26 Hz, 1 H) 8.39 (s, 1 H) |
| | | | | 9.49 (br. s., 1 H) 10.42 (br. s., 1 H) |
| 301 | 3-(6-(5-isopropylpyridin-2- | 0.98^{c} | 497.0 (M + H)⁺ | ¹H NMR (400 MHz, DMSO-d₆) δ ppm |
| | ylamino)pyrimidin-4-ylamino)-N- | | | 1.19 (d, J = 6.84 Hz, 6 H) 2.41 (d, |
| | methyl-4-(2,2,2- | | | J = 5.07 Hz, 3 H) 2.86-2.96 (m, 1 H) |
| | trifluoroethoxy)benzenesulfonamide | | | 4.89 (q, J = 8.82 Hz, 2 H) 6.81 (br. s., 1 |
| | trifluoroacetate | | | H) 7.31 (d, J = 8.60 Hz, 1 H) 7.42 (d, |
| | | | | J = 8.38 Hz, 2 H) 7.60 (dd, J = 9.04, |
| | | | | 1.98 Hz, 1 H) 7.80 (dd, J = 8.93, 2.10 Hz, |
| | | | | 1 H) 7.97 (d, J = 1.98 Hz, 1 H) |
| | | | | 8.16 (d, J = 2.21 Hz, 1 H) 8.38 (s, 1 H) |
| | | | | 9.51 (br. s., 1 H) 10.84 (br. s., 1 H) |
| 302 | 3-({6-[(5-chloro-2- | 2.03^{a} | 409.0 (M + H)⁺ | ¹H NMR (400 MHz, DMSO-d₆) δ ppm |
| | pyridinyl)amino]-4- | | | 2.46 (d, J = 5.02 Hz, 3 H) 7.32 (s, 1 H) |
| | pyrimidinyl}amino)-4-fluoro-N- | | | 7.48-7.54 (m, 3 H) 7.58 (d, J = 9.03 Hz, |
| | methylbenzenesulfonamide | | | 1 H) 7.84 (dd, J = 8.78, 2.76 Hz, 1 |
| | trifluoroacetate | | | H) 8.30 (d, J = 2.76 Hz, 1 H) 8.38 (s, 1 |
| | | | | H) 8.45 (d, J = 7.28 Hz, 1 H) 9.59 (br. |
| | | | | s., 1 H) 10.25 (br. s., 1 H) |
| 303 | 4-fluoro-N-methyl-3-[(6-{[5- | 1.59^{a} | 443.1 (M + H)⁺ | ¹H NMR (500 MHz, DMSO-d₆) δ ppm |
| | (trifluoromethyl)-2- | | | 2.46 (d, J = 4.88 Hz, 3 H) |
| | pyridinyl]amino}-4- | | | 7.44-7.53 (m, 4 H) 7.73 (d, J = 8.79 Hz, 1 H) |
| | pyrimidinyl)amino]benzenesulfonamide | | | 8.06 (dd, J = 8.91, 2.32 Hz, 1 H) 8.41 (s, 1 |
| | trifluoroacetate | | | H) 8.46 (d, J = 7.08 Hz, 1 H) 8.60 (s, 1 |
| | | | | H) 9.56 (s, 1 H) 10.47 (s, 1 H) |
| 304 | 4-chloro-3-({6-[(5-chloro-2- | 1.53^{a} | 425.0 (M + H)⁺ | ¹H NMR (500 MHz, DMSO-d₆) δ ppm |
| | pyridinyl)amino]-4- | | | 2.46 (d, J = 4.88 Hz, 3 H) 7.29 (s, 1 H) |
| | pyrimidinyl}amino)-N- | | | 7.51 (dd, J = 8.42, 2.08 Hz, 1 H) |
| | methylbenzenesulfonamide | | | 7.55 (d, J = 5.13 Hz, 1 H) 7.59 (d, J = 9.03 Hz, |
| | trifluoroacetate | | | 1 H) 7.74 (d, J = 8.55 Hz, 1 H) |
| | | | | 7.81 (dd, J = 9.03, 2.69 Hz, 1 H) 8.19 (d, |
| | | | | J = 2.20 Hz, 1 H) 8.27 (d, J = 2.44 Hz, 1 |
| | | | | H) 8.31 (s, 1 H) 9.27 (br. s., 1 H) |
| | | | | 10.13 (s, 1 H) |
| 305 | 3-({6-[(5-chloro-2- | 2.01^{a} | 469.0 (M + H)⁺ | ¹H NMR (400 MHz, DMSO-d₆) δ ppm |
| | pyridinyl)amino]-4- | | | 2.50 (d, 3H, obscured by solvent) 3.33 |
| | pyrimidinyl}amino)-N-methyl-4- | | | 9s, 3H) 7.42 (s, 1 H) 7.61 (d, J = 9.03 Hz, |
| | (methylsulfonyl)benzenesulfonamide | | | 1 H) 7.68 (dd, J = 8.28, 1.76 Hz, 1 |
| | | | | H) 7.80 (q, J = 4.52 Hz, 1 H) 7.84 (dd, |
| | | | | J = 9.03, 2.76 Hz, 1 H) 8.12 (d, J = 8.28 Hz, |
| | | | | 1 H) 8.31-8.34 (m, 1 H) |
| | | | | 8.38-8.40 (m, 1 H) 8.45-8.48 (m, 1 H) |
| | | | | 9.08-9.10 (m, 1 H) 10.26 (s, 1 H) |
| 306 | N-methyl-4-(methylsulfonyl)-3- | 2.35^{a} | 503.0 (M + H)⁺ | ¹H NMR (400 MHz, METHANOL-d₄) δ |
| | [(6-{[5-(trifluoromethyl)-2- | | | ppm 2.64 (s, 3 H) 3.22 (s, 3 H) |
| | pyridinyl]amino}-4- | | | 7.57 (d, J = 8.78 Hz, 1 H) 7.72 (dd, J = 8.28, |
| | pyrimidinyl)amino]benzenesulfonamide | | | 1.76 Hz, 1 H) 7.79 (d, J = 0.75 Hz, 1 H) |
| | | | | 7.96 (dd, J = 8.78, 2.51 Hz, 1 H) |
| | | | | 8.17 (d, J = 8.28 Hz, 1 H) 8.46 (d, J = 0.75 Hz, |
| | | | | 1 H) 8.66 (br. s., 1 H) 8.76 (d, |
| | | | | J = 1.51 Hz, 1 H) |
| 307 | N-methyl-4-(methylsulfonyl)-3- | 0.78^{c} | 485.0 (M + H)⁺ | ¹H NMR (400 MHz, DMSO-d₆) δ ppm |
| | {[6-(6-quinolinylamino)-4- | | | 2.47 (d, 3H, obscured by solvent) |
| | pyrimidinyl]amino}benzenesulfonamide | | | 3.30 (s, 3 H) 6.43 (d, J = 2.43 Hz, 1 H) |
| | | | | 7.64-7.73 (m, 1 H) 7.75-7.87 (m, 2 H) |
| | | | | 8.04-8.18 (m, 3 H) 8.34-8.47 (m, 2 |
| | | | | H) 8.60 (d, J = 1.54 Hz, 1 H) 8.80 (d, |
| | | | | J = 9.04 Hz, 1 H) 8.98 (d, J = 4.85 Hz, 1 |
| | | | | H) 9.11 (br. s., 1 H) 10.11 (s, 1 H) |
| 308 | 3-({6-[(5-chloro-2- | 1.64^{a} | 503.2 (M + H)⁺ | ¹H NMR (400 MHz, DMSO-d₆) δ ppm |
| | pyridinyl)amino]-4- | | | 1.44 (d, J = 6.27 Hz, 3 H) 2.45 (d, |
| | pyrimidinyl}amino)-N-methyl-4- | | | J = 4.77 Hz, 3 H) 5.37-5.49 (m, 1 H) |
| | [(2,2,2-trifluoro-1- | | | 7.08 (br. s., 1 H) 7.43 (q, J = 4.77 Hz, 1 |
| | methylethyl)oxy]benzenesulfonamide | | | H) 7.47-7.55 (m, 2 H) 7.59 (dd, |
| | trifluoroacetate | | | J = 8.78, 2.01 Hz, 1 H) 7.86 (dd, |
| | | | | J = 8.78, 2.76 Hz, 1 H) 8.05 (d, J = 1.76 Hz, |
| | | | | 1 H) 8.29 (d, J = 2.76 Hz, 1 H) |
| | | | | 8.36 (s, 1 H) 9.22 (br. s., 1 H) 10.38 (br. s., |
| | | | | 1 H) |
| 309 | N-methyl-4-[(2,2,2-trifluoro-1- | 2.32^{a} | 537.1 (M + H)⁺ | ¹H NMR (400 MHz, DMSO-d₆) δ ppm |
| | methylethyl)oxy]-3-[(6-{[5- | | | 1.44 (d, J = 6.27 Hz, 3 H) 2.44 (d, |
| | (trifluoromethyl)-2- | | | J = 4.52 Hz, 3 H) 5.42 (dt, 1 H) |
| | pyridinyl]amino}-4- | | | 7.28-7.33 (m, 1 H) 7.38-7.45 (m, 1 H) |
| | pyrimidinyl)amino]benzenesulfonamide | | | 7.44-7.50 (m, 1 H) 7.51-7.56 (m, 1 |
| | | | | H) 7.76 (d, J = 9.04 Hz, 1 H) 8.05 (dd, |
| | | | | J = 8.91, 2.38 Hz, 1 H) 8.13 (d, J = 2.01 Hz, |
| | | | | 1 H) 8.33 (s, 1 H) 8.59 (s, 1 H) |
| | | | | 8.57-8.62 (m, 1 H) 8.85 (s, 1 H) |
| | | | | 10.35 (s, 1 H) |
| 310 | 4-(tert-butylsulfonyl)-N-methyl-3- | 1.14^{c} | 544.9 (M + H)⁺ | ¹H NMR (400 MHz, DMSO-d₆) δ ppm |
| | (6-(5-(trifluoromethyl)pyridin-2- | | | 1.23 (s, 9 H) 2.47 (d, 3H, obscured by |
| | ylamino)pyrimidin-4- | | | solvent) 7.57 (s, 1 H) 7.60 (dd, 1 H) |
| | ylamino)benzenesulfonamide | | | 7.68 (d, J = 9.04 Hz, 1 H) 7.80 (q, |
| | trifluoroacetate | | | J = 5.00 Hz, 1 H) 8.05 (dd, J = 8.93, |
| | | | | 2.54 Hz, 1 H) 8.45 (s, 1 H) 8.61 (d, |
| | | | | J = 1.54 Hz, 1 H) 8.66 (s, 1 H) 9.32 (s, |
| | | | | 1 H) 10.56 (s, 1 H) |
| 311 | 4-(tert-butylsulfonyl)-3-(6-(5- | 1.17^{c} | 511.2 (M + H)⁺ | ¹H NMR (400 MHz, DMSO-d₆) δ ppm |
| | chloropyridin-2- | | | 1.22 (s, 9 H) 2.47 (d, 3H, obscured by |
| | ylamino)pyrimidin-4-ylamino)-N- | | | solvent) 7.40 (s, 1 H) 7.55-7.60 (m, 2 |
| | methylbenzenesulfonamide | | | H) 7.76-7.83 (m, 2 H) 7.98 (d, J = 8.38 Hz, |
| | trifluoroacetate | | | 1 H) 8.32 (d, J = 2.43 Hz, 1 H) |
| | | | | 8.40 (s, 1 H) 8.61 (d, J = 1.54 Hz, 1 H) |
| | | | | 9.28 (s, 1 H) 10.27 (s, 1 H) |
| 312 | N-methyl-4-(propane-2-sulfonyl)- | 1.23^{c} | 530.9 (M + H)⁺ | ¹H NMR (400 MHz, DMSO-d₆) δ ppm |
| | 3-[6-(5-trifluoromethyl-pyridin-2- | | | 1.15 (d, J = 6.62 Hz, 6 H) 2.47 (d, 3H, |
| | ylamino)-pyrimidin-4-ylamino]- | | | obscured by solvent) 3.47-3.56 (m, 1 |
| | benzenesulfonamide | | | H) 7.51 (s, 1 H) 7.62-7.74 (m, 2 H) |
| | | | | 7.80 (q, J = 4.92 Hz, 1 H) |
| | | | | 8.02-8.08 (m, 2 H) 8.43 (d, J = 0.88 Hz, 1 H) |
| | | | | 8.45-8.48 (m, 1 H) 8.63 (d, J = 2.43 Hz, 1 |
| | | | | H) 9.20 (br. s., 1 H) 10.60 (s, 1 H) |
| 313 | 3-[6-(5-chloro-pyridin-2- | 1.00^{c} | 496.9 (M + H)⁺ | ¹H NMR (400 MHz, DMSO-d₆) δ ppm |
| | ylamino)-pyrimidin-4-ylamino]-N- | | | 1.14 (d, J = 6.62 Hz, 6 H) 2.47 (d, 3H, |
| | methyl-4-(propane-2-sulfonyl)- | | | obscured by solvent) 3.45-3.54 (m, 1 |
| | benzenesulfonamide | | | H) 7.28 (s, 1 H) 7.52 (d, J = 8.82 Hz, 1 |
| | | | | H) 7.69 (dd, J = 8.27, 1.65 Hz, 1 H) |
| | | | | 7.78 (q, 1 H) 7.84 (dd, J = 8.82, 2.65 Hz, |
| | | | | 1 H) 8.06 (d, J = 8.38 Hz, 1 H) |
| | | | | 8.30 (d, J = 2.65 Hz, 1 H) 8.39 (d, J = 1.54 Hz, |
| | | | | 1 H) 8.41 (s, 1 H) 9.36 (br. s., 1 H) |
| | | | | 10.49 (br. s., 1 H) |
| 314 | 3-({6-[(5-chloro-2- | 2.17^{a} | 475.0 (M + H)⁺ | ¹H NMR (400 MHz, DMSO-d₆) δ ppm |
| | pyridinyl)amino]-4- | | | 2.49 (d, J = 4.77 Hz, 3 H) 7.33 (s, 1 H) |
| | pyrimidinyl}amino)-N-methyl-4- | | | 7.54-7.68 (m, 4 H) 7.85 (dd, J = 8.91, |
| | [(trifluoromethyl)oxy]benzenesulfonamide | | | 2.64 Hz, 1 H) 8.31 (d, J = 2.26 Hz, 1 H) |
| | trifluoroacetate | | | 8.37 (s, 1 H) 8.44 (d, J = 2.01 Hz, 1 H) |
| | | | | 9.62 (s, 1 H) 10.31 (br. s., 1 H) |
| 315 | 1-[6-(5-chloro-pyridin-2- | 0.98^{c} | 445.1 (M + H)⁺ | ¹H NMR (400 MHz, DMSO-d₆) δ ppm |
| | ylamino)-pyrimidin-4-yl]-3,3- | | | 1.35 (s, 6 H) 2.40 (d, J = 4.85 Hz, 3 H) |
| | dimethyl-2,3-dihydro-1H-indole- | | | 7.20 (br. s., 1 H) 7.34-7.39 (m, 2 H) |
| | 6-sulfonic acid methylamide | | | 7.45 (d, J = 7.94 Hz, 1 H) 7.62 (d, |
| | trifluoroacetate | | | J = 8.60 Hz, 1 H) 7.82 (dd, J = 8.82, |
| | | | | 2.65 Hz, 1 H) 8.35 (d, J = 2.43 Hz, 1 H) |
| | | | | 8.48 (s, 1 H) 8.69 (s, 1 H) 10.27 (br. |
| | | | | s., 1 H) |
| 316 | 5-(6-(5-chloropyridin-2- | 0.96^{c} | 521.0 (M + H)⁺ | ¹H NMR (400 MHz, DMSO-d₆) δ ppm |
| | ylamino)pyrimidin-4-ylamino)-2- | | | 1.39 (d, J = 6.39 Hz, 3 H) 2.47 (d, 3 H, |
| | fluoro-N-methyl-4-(1,1,1- | | | obscured by solvent) 5.38-5.47 (m, 1 |
| | trifluoropropan-2- | | | H) 6.98 (br. s., 1 H) 7.52 (m, J = 12.13 Hz, |
| | yloxy)benzenesulfonamide | | | 2 H) 7.68 (d, J = 4.19 Hz, 1 H) |
| | trifluoroacetate | | | 7.81 (dd, J = 8.82, 2.43 Hz, 1 H) 7.87 (d, |
| | | | | J = 7.72 Hz, 1 H) 8.25 (s, 1 H) 8.30 (s, |
| | | | | 1 H) 9.19 (br. s., 1 H) 10.31 (br. s., 1 |
| | | | | H) |
| 317 | 5-[6-(5-chloro-pyridin-2- | 0.89^{c} | 487.0 (M + H)⁺ | ¹H NMR (400 MHz, DMSO-d₆) δ ppm |
| | ylamino)-pyrimidin-4-ylamino]-2- | | | 2.57 (d, J = 4.63 Hz, 3 H) 3.32 (s, 3 H) |
| | fluoro-4-methanesulfonyl-N- | | | 7.28-7.31 (m, 1 H) 7.57 (d, 1 H) |
| | methyl-benzenesulfonamide | | | 7.81 (dd, J = 8.82, 2.65 Hz, 2 H) 7.90 (d, |
| | trifluoroacetate | | | J = 9.04 Hz, 1 H) 8.08 (m, J = 14.11 Hz, |
| | | | | 1 H) 8.25-8.30 (m, 2 H) |
| | | | | 8.31-8.33 (m, 1 H) 9.07 (br. s., 1 H) 10.25 (s, 1 |
| | | | | H) |
| 318 | 5-({6-[(5-chloro-2- | 2.10^{a} | 507.0 (M + H)⁺ | ¹H NMR (400 MHz, DMSO-d₆) δ ppm |
| | pyridinyl)amino]-4- | | | 2.47 (d, 3 H, obscured by solvent) |
| | pyrimidinyl}amino)-2-fluoro-N- | | | 4.99 (q, J = 8.70 Hz, 2 H) 7.09 (br. s., 1 |
| | methyl-4-[(2,2,2- | | | H) 7.53 (d, J = 11.80 Hz, 1 H) 7.60 (d, |
| | trifluoroethyl)oxy]benzenesulfonamide | | | J = 8.78 Hz, 1 H) 7.77 (q, J = 4.94 Hz, 1 |
| | trifluoroacetate | | | H) 7.90 (dd, J = 8.91, 2.64 Hz, 1 H) |
| | | | | 7.97 (d, J = 7.78 Hz, 1 H) 8.35 (d, |
| | | | | J = 2.51 Hz, 1 H) 8.37 (s, 1 H) 9.29 (br. |
| | | | | s., 1 H) 10.33 (br. s., 1 H) |
| 319 | 2-fluoro-N-methyl-4-[(2,2,2- | 2.23^{a} | 541.1 (M + H)⁺ | ¹H NMR (400 MHz, METHANOL-d₄) δ |
| | trifluoroethyl)oxy]-5-[(6-{[5- | | | ppm 2.65 (s, 3 H) 4.80 (q, J = 8.28 Hz, |
| | (trifluoromethyl)-2- | | | 2 H) 6.67 (br. s., 1 H) 7.30 (d, J = 8.78 Hz, |
| | pyridinyl]amino}-4- | | | 1 H) 7.37 (d, J = 11.29 Hz, 1 H) |
| | pyrimidinyl)amino]benzenesulfonamide | | | 8.01-8.05 (m, 1 H) 8.14 (dd, J = 8.78, |
| | trifluoroacetate | | | 2.26 Hz, 1 H) 8.52 (s, 1 H) 8.71 (s, 1 |
| | | | | H) |
| 320 | 3-({6-[(5-fluoro-2- | 1.50^{a} | 473.1 (M + H)⁺ | ¹H NMR (400 MHz, DMSO-d₆) δ ppm |
| | pyridinyl)amino]-4- | | | 2.44 (d, J = 5.02 Hz, 3 H) 4.92 (q, |
| | pyrimidinyl}amino)-N-methyl-4- | | | J = 8.78 Hz, 2 H) 7.00 (br. s., 1 H) |
| | [(2,2,2- | | | 7.41-7.50 (m, 3 H) 7.63 (dd, J = 8.66, 2.13 Hz, |
| | trifluoroethyl)oxy]benzenesulfonamide | | | 1 H) 7.77 (td, J = 8.72, 3.14 Hz, 1 |
| | trifluoroacetate | | | H) 8.00 (d, J = 2.01 Hz, 1 H) 8.28 (d, |
| | | | | J = 3.26 Hz, 1 H) 8.39 (s, 1 H) 9.56 (br. |
| | | | | s., 1 H) 10.53 (br. s., 1 H) |
| 321 | 3-({6-[(5-chloro-2- | 1.54^{a} | 483.1 (M + H)⁺ | ¹H NMR (400 MHz, DMSO-d₆) δ ppm |
| | pyridinyl)amino]-4- | | | 1.12 (t, J = 7.40 Hz, 3 H) 2.47 (d, 3H, |
| | pyrimidinyl}amino)-4- | | | obscured by solvent) 3.41 (q, J = 7.28 Hz, |
| | (ethylsulfonyl)-N- | | | 2 H) 7.37 (br. s., 1 H) 7.60 (d, 1 |
| | methylbenzenesulfonamide | | | H) 7.71 (dd, J = 8.28, 1.51 Hz, 1 H) |
| | trifluoroacetate | | | 7.80 (q, J = 4.52 Hz, 1 H) 7.86 (dd, |
| | | | | J = 8.91, 2.64 Hz, 1 H) 8.10 (d, J = 8.28 Hz, |
| | | | | 1 H) 8.33 (d, J = 2.51 Hz, 1 H) |
| | | | | 8.41 (s, 1 H) 8.45 (s, 1 H) 9.21 (br. s., 1 H) |
| | | | | 10.35 (br. s., 1 H) |
| 322 | 4-(ethylsulfonyl)-N-methyl-3-[(6- | 2.36^{a} | 517.1 (M + H)⁺ | ¹H NMR (400 MHz, DMSO-d₆) δ ppm |
| | {[5-(trifluoromethyl)-2- | | | 1.12 (t, J = 7.28 Hz, 3 H) 2.50 (d, 3H, |
| | pyridinyl]amino}-4- | | | obscured by solvent) 3.41 (q, J = 7.45 Hz, |
| | pyrimidinyl)amino]benzenesulfonamide | | | 2 H) 7.53 (s, 1 H) 7.69-7.76 (m, |
| | trifluoroacetate | | | 2 H) 7.80 (q, J = 4.94 Hz, 1 H) |
| | | | | 8.07-8.13 (m, 2 H) 8.43-8.48 (m, 2 H) |
| | | | | 8.66 (s, 1 H) 9.20 (br. s., 1 H) |
| | | | | 10.60 (s, 1 H) |
| 323 | 3-({6-[(5-cyano-2- | 1.41^{a} | 460.1 (M + H)⁺ | ¹H NMR (400 MHz, DMSO-d₆) δ ppm |
| | pyridinyl)amino]-4- | | | 2.50 (d, 3H, obscured by solvent) |
| | pyrimidinyl}amino)-N-methyl-4- | | | 3.33 (s, 3 H) 7.51 (s, 1 H) 7.67-7.74 (m, 2 |
| | (methylsulfonyl)benzenesulfonamide | | | H) 7.80 (q, J = 4.94 Hz, 1 H) |
| | trifluoroacetate | | | 8.11-8.17 (m, 2 H) 8.41-8.45 (m, 2 H) |
| | | | | 8.74 (d, J = 1.76 Hz, 1 H) 9.22 (s, 1 H) |
| | | | | 10.65 (s, 1 H) |
| 324 | 3-({6-[(5-cyano-2- | 1.66^{a} | 494.2 (M + H)⁺ | ¹H NMR (400 MHz, DMSO-d₆) δ ppm |
| | pyridinyl)amino]-4- | | | 1.43 (d, J = 6.27 Hz, 3 H) 2.45 (d, |
| | pyrimidinyl}amino)-N-methyl-4- | | | J = 5.02 Hz, 3 H) 5.36-5.47 (m, 1 H) |
| | [(2,2,2-trifluoro-1- | | | 7.27 (s, 1 H) 7.42 (q, J = 5.02 Hz, 1 H) |
| | methylethyl)oxy]benzenesulfonamide | | | 7.46-7.51 (m, 1 H) 7.55 (dd, J = 8.53, |
| | trifluoroacetate | | | 2.26 Hz, 1 H) 7.69 (d, J = 8.78 Hz, 1 H) |
| | | | | 8.09 (d, J = 2.01 Hz, 1 H) 8.11 (dd, 1 |
| | | | | H) 8.35 (s, 1 H) 8.69 (d, J = 1.76 Hz, 1 |
| | | | | H) 9.06 (br. s., 1 H) 10.52 (s, 1 H) |
| 325 | 2-{[6-({3- | 5.65^{b} | 407.0 (M + H)⁺ | ¹H NMR (400 MHz, DMSO-d6) δ |
| | [(methylamino)sulfonyl]phenyl}amino)- | | | ppm 11.33 (br. s., 1H), 9.85 (s, 1H), |
| | 4-pyrimidinyl]amino}-1,3- | | | 8.50 (s, 1H), 8.11-8.22 (m, 1H), |
| | thiazole-5-carboxylic acid | | | 7.89-8.00 (m, 1H), 7.52 (t, J = 8.03 Hz, |
| | | | | 2H), 7.45 (q, J = 4.94 Hz, 1H), |
| | | | | 7.35 (d, J = 7.78 Hz, 1H), 6.59 (s, 1H), |
| | | | | 2.45 (d, J = 5.02 Hz, 3H) |
| 326 | (2-{[6-({3- | 0.99^{d} | 421.0 (M + H)⁺ | ¹H NMR (400 MHz, DMSO-d6) δ ppm |
| | [(methylamino)sulfonyl]phenyl}amino)- | | | 11.59 (br. s., 1 H), 9.89 (br. s., 1 H), |
| | 4-pyrimidinyl]amino}-1,3- | | | 8.48 (s, 1 H), 8.10 (br. s., 1 H), |
| | thiazol-4-yl)acetic acid | | | 7.87 (m, 1 H), 7.50 (m, 1 H), 7.43 (m, 1H), |
| | | | | 7.35 (m, 1 H), 6.83 (s, 1 H), 6.44 (s, 1 |
| | | | | H), 3.56 (s, 2H), 2.39-2.45 (m, 3 H) |
| 327 | 1-{6-[(4-chlorophenyl)amino]-4- | 2.91^{a} | 414.1 (M + H)⁺ | ¹H NMR (400 MHz, DMSO-d₆) δ ppm |
| | pyrimidinyl}-N-methyl-1H-indole- | | | 2.41 (d, J = 5.02 Hz, 3 H) |
| | 6-sulfonamide trifluoroacetate | | | 6.95-6.99 (m, 2 H) 7.40-7.47 (m, 3 H) 7.62 (dd, |
| | | | | J = 8.28, 1.51 Hz, 1 H) 7.75 (d, J = 8.78 Hz, |
| | | | | 2 H) 7.87 (d, J = 8.28 Hz, 1 H) |
| | | | | 8.19 (d, J = 3.76 Hz, 1 H) 8.74 (s, 1 H) |
| | | | | 8.97 (s, 1 H) 10.00 (s, 1 H) |
| 328 | 3-{6-[(4-chlorophenyl)amino]-4- | 2.59^{a} | 431.0 (M + H)⁺ | ¹H NMR (400 MHz, DMSO-d₆) δ ppm |
| | pyrimidinyl}-N-methyl-2-oxo-2,3- | | | 2.46 (d, J = 5.02 Hz, 3 H) 7.32 (d, 1 H) |
| | dihydro-1H-benzimidazole-5- | | | 7.43-7.50 (m, 3 H) 7.66 (dd, J = 8.03, |
| | sulfonamide trifluoroacetate | | | 1.76 Hz, 1 H) 7.83 (d, J = 7.78 Hz, 3 H) |
| | | | | 8.79 (s, 1 H) 8.81 (d, J = 1.51 Hz, 1 H) |
| | | | | 10.11 (s, 1 H) 12.00 (s, 1 H) |
| 329 | 3-{[6-({3-[6-(dimethylamino)-3- | 1.60^{c} | 476.0 (M + H)⁺ | ¹H NMR (400 MHz, DMSO-d6) δ ppm |
| | pyridinyl]phenyl}amino)-4- | | | 9.55 (s, 1H), 9.29 (s, 1H), |
| | pyrimidinyl]amino}-N- | | | 8.39-8.46 (m, 1H), 8.35 (s, 1H), 8.10 (br. s., 1H), |
| | methylbenzenesulfonamide | | | 7.93 (d, J = 7.78 Hz, 1H), 7.80 (dd, J = 2.26, |
| | | | | 8.78 Hz, 1H), 7.74 (br. s., 1H), |
| | | | | 7.45-7.54 (m, 2H), 7.39-7.45 (m, |
| | | | | 1H), 7.30-7.39 (m, 2H), 7.23 (d, J = 7.53 Hz, |
| | | | | 1H), 6.75 (d, J = 8.78 Hz, |
| | | | | 1H), 6.25 (s, 1H), 3.08 (s, 6H), |
| | | | | 2.45 (d, J = 5.02 Hz, 3H) |
| 330 | N-methyl-3-({6-[(5-methyl-3- | 2.31^{a} | 446.1 (M + H)⁺ | ¹H NMR (400 MHz, DMSO-d6) δ ppm |
| | biphenylyl)amino]-4- | | | 9.82 (br. s., 1H), 9.59 (br. s., 1H), |
| | pyrimidinyl}amino)benzenesulfonamide | | | 8.41 (s, 1H), 8.04 (br. s., 1H), 7.89 (d, J = 7.78 Hz, |
| | trifluoroacetate | | | 1H), 7.65 (d, J = 7.53 Hz, |
| | | | | 2H), 7.43-7.61 (m, 5H), |
| | | | | 7.33-7.43 (m, 3H), 7.21 (br. s., 1H), 6.24 (s, 1H), |
| | | | | 2.42-2.47 (m, 3H), 2.39 (s, 3H) |
| 331 | N-methyl-3-[(6-{[3-methyl-5-(3- | 1.88^{a} | 447.1 (M + H)⁺ | ¹H NMR (400 MHz, DMSO-d6) δ ppm |
| | pyridinyl)phenyl]amino}-4- | | | 9.72 (s, 1H), 9.54 (s, 1H), 9.02 (br. s., |
| | pyrimidinyl)amino]benzenesulfonamide | | | 1H), 8.72 (d, J = 4.27 Hz, 1H), 8.39 (s, |
| | trifluoroacetate | | | 1H), 8.36 (d, J = 7.78 Hz, 1H), 8.07 (s, |
| | | | | 1H), 7.90-7.96 (m, 1H), |
| | | | | 7.73-7.80 (m, 2H), 7.53 (t, J = 8.03 Hz, 1H), |
| | | | | 7.43-7.49 (m, 2H), 7.36 (d, J = 7.78 Hz, |
| | | | | 1H), 7.28 (s, 1H), 6.25 (s, 1H), |
| | | | | 2.44 (d, J = 4.77 Hz, 3H), 2.40 (s, 3H) |
| 332 | 3-[(6-{[3′-(dimethylamino)-3- | 1.72^{c} | 475.0 (M + H)⁺ | ¹H NMR (400 MHz, DMSO-d6) δ ppm |
| | biphenylyl]amino}-4- | | | 9.55 (s, 1H), 9.32 (s, 1H), 8.35 (s, |
| | pyrimidinyl)amino]-N- | | | 1H), 8.11 (s, 1H), 7.92 (d, J = 8.03 Hz, |
| | methylbenzenesulfonamide | | | 1H), 7.76 (s, 1H), 7.60 (d, J = 8.03 Hz, |
| | | | | 1H), 7.50 (t, J = 8.03 Hz, 1H), |
| | | | | 7.35-7.45 (m, 2H), 7.32 (d, J = 7.78 Hz, |
| | | | | 1H), 7.24-7.30 (m, 2H), |
| | | | | 6.89-6.94 (m, 2H), 6.72-6.78 (m, 1H), 6.24 (s, |
| | | | | 1H), 2.97 (s, 6H), 2.45 (d, J = 3.76 Hz, |
| | | | | 3H) |
| 333 | N-methyl-3-[(6-{[4′-(4- | 1.60^{c} | 517.0 (M + H)⁺ | ¹H NMR (400 MHz, DMSO-d6) δ ppm |
| | morpholinyl)-3- | | | 9.80 (s, 1H), 9.54 (s, 1H), 8.58 (s, |
| | biphenylyl]amino}-4- | | | 1H), 8.33 (s, 1H), 8.16 (d, J = 8.78 Hz, |
| | pyrimidinyl)amino]benzenesulfonamide | | | 1H), 7.98 (br. s., 1H), 7.70-7.81 (m, |
| | | | | 4H), 7.66 (q, J = 4.60 Hz, 1H), |
| | | | | 7.53-7.63 (m, 2H), 7.47 (d, J = 7.28 Hz, |
| | | | | 1H), 7.28 (d, J = 8.78 Hz, 2H), 6.48 (s, |
| | | | | 1H), 3.96-4.03 (m, 4H), |
| | | | | 3.37-3.45 (m, 4H), 2.68 (d, J = 5.02 Hz, 3H) |
| 334 | N-methyl-3-{[6-({3-[6- | 1.60^{c} | 463.0 (M + H)⁺ | ¹H NMR (400 MHz, DMSO-d6) δ ppm |
| | (methyloxy)-3- | | | 9.56 (s, 1H), 9.36 (s, 1H), 8.47 (d, J = 2.01 Hz, |
| | pyridinyl]phenyl}amino)-4- | | | 1H), 8.36 (s, 1H), 8.09 (s, |
| | pyrimidinyl]amino}benzenesulfonamide | | | 1H), 7.99 (dd, J = 2.51, 8.53 Hz, 1H), |
| | | | | 7.93 (d, J = 8.28 Hz, 1H), 7.81 (s, 1H), |
| | | | | 7.57 (d, J = 7.53 Hz, 1H), 7.51 (t, J = 8.03 Hz, |
| | | | | 1H), 7.36-7.46 (m, 2H), |
| | | | | 7.33 (d, J = 7.53 Hz, 1H), 7.28 (d, J = 7.53 Hz, |
| | | | | 1H), 6.94 (d, J = 8.78 Hz, |
| | | | | 1H), 6.25 (s, 1H), 3.91 (s, 3H), |
| | | | | 2.44 (d, J = 5.02 Hz, 3H) |
| 335 | 3′-{[6-({3- | 1.42^{c} | 475.0 (M + H)⁺ | ¹H NMR (400 MHz, DMSO-d6) δ ppm |
| | [(methylamino)sulfonyl]phenyl}amino)- | | | 9.59 (s, 1H), 9.39 (s, 1H), 8.36 (s, |
| | 4-pyrimidinyl]amino}-4- | | | 1H), 8.10 (s, 1H), 8.03 (br. s., 1H), |
| | biphenylcarboxamide | | | 7.98 (d, J = 8.03 Hz, 2H), 7.93 (d, J = 8.03 Hz, |
| | | | | 1H), 7.88 (s, 1H), 7.73 (d, J = 8.03 Hz, |
| | | | | 2H), 7.64 (d, J = 8.03 Hz, |
| | | | | 1H), 7.29-7.54 (m, 6H), 6.25 (s, 1H), |
| | | | | 2.44 (s, 3H) |
| 336 | N-methyl-3-{[6-({3-[5- | 1.51^{c} | 463.0 (M + H)⁺ | ¹H NMR (400 MHz, DMSO-d6) δ ppm |
| | (methyloxy)-3- | | | 9.57 (s, 1 H), 9.39 (s, 1 H), 8.47 (d, J = 1.76 Hz, |
| | pyridinyl]phenyl}amino)-4- | | | 1H), 8.37 (s, 1H), 8.32 (d, J = 3.26 Hz, |
| | pyrimidinyl]amino}benzenesulfonamide | | | 1H), 8.10 (s, 1H), 7.93 (dd, J = 1.51, |
| | | | | 8.28 Hz, 1H), 7.87 (s, 1H), |
| | | | | 7.67 (d, J = 8.03 Hz, 1H), |
| | | | | 7.57-7.61 (m, 1H), 7.39-7.54 (m, 3H), |
| | | | | 7.31-7.39 (m, 2H), 6.26 (s, 1H), 3.92 (s, |
| | | | | 3H), 2.45 (d, J = 5.02 Hz, 3H) |
| 337 | 3′-{[6-({3- | 1.41^{c} | 475.2 (M + H)⁺ | ¹H NMR (400 MHz, DMSO-d6) δ ppm |
| | [(methylamino)sulfonyl]phenyl}amino)- | | | 9.56 (s, 1H), 9.37 (s, 1H), 8.36 (s, |
| | 4-pyrimidinyl]amino}-3- | | | 1H), 8.16 (s, 1H), 8.10 (d, J = 1.76 Hz, |
| | biphenylcarboxamide | | | 2H), 7.92 (dd, J = 1.51, 8.28 Hz, 1H), |
| | | | | 7.88 (d, J = 7.78 Hz, 1H), 7.84 (s, 1H), |
| | | | | 7.80 (d, J = 7.78 Hz, 1H), 7.67 (d, J = 8.03 Hz, |
| | | | | 1H), 7.57 (t, J = 7.65 Hz, |
| | | | | 1H), 7.50 (t, J = 8.03 Hz, 1H), |
| | | | | 7.39-7.47 (m, 3H), 7.33 (d, J = 8.03 Hz, |
| | | | | 1H), 7.36 (d, J = 8.03 Hz, 1H), 6.24 (s, |
| | | | | 1H), 2.42-2.48 (m, 3H) |
| 338 | N-methyl-3-{[6-({3′- | 1.49^{c} | 525.1 (M + H)⁺ | ¹H NMR (400 MHz, DMSO-d6) δ ppm |
| | [(methylsulfonyl)amino]-3- | | | 9.86 (s, 1H), 9.56 (s, 1H), 9.39 (s, |
| | biphenylyl}amino)-4- | | | 1H), 8.36 (s, 1H), 8.10 (t, J = 1.76 Hz, |
| | pyrimidinyl]amino}benzenesulfonamide | | | 1H), 7.92 (dd, J = 1.51, 8.03 Hz, 1H), |
| | | | | 7.81 (s, 1 H), 7.63 (d, J = 8.03 Hz, 1H), |
| | | | | 7.36-7.54 (m, 6H), 7.33 (d, J = 7.78 Hz, |
| | | | | 1H), 7.23 (d, J = 7.78 Hz, 2H), |
| | | | | 6.24 (s, 1H), 3.05 (s, 3H), 2.45 (d, J = 5.02 Hz, |
| | | | | 3H) |
| 339 | 3-[(6-{[4′-(dimethylamino)-3- | 1.68^{c} | 475.2 (M + H)⁺ | ¹H NMR (400 MHz, DMSO-d6) δ ppm |
| | biphenylyl]amino}-4- | | | 9.54 (s, 1H), 9.26 (s, 1H), 8.34 (s, |
| | pyrimidinyl)amino]-N- | | | 1H), 8.10 (s, 1H), 7.92 (d, J = 8.28 Hz, |
| | methylbenzenesulfonamide | | | 1H), 7.71 (s, 1H), 7.44-7.54 (m, 4H), |
| | | | | 7.41 (br. s., 1H), 7.30-7.38 (m, 2H), |
| | | | | 7.21 (s, 1H), 6.82 (d, J = 8.53 Hz, 2H), |
| | | | | 6.24 (s, 1H), 2.95 (s, 6H), |
| | | | | 2.42-2.47 (m, 3H) |
| 340 | N-methyl-3-{[6-({3-[4- | 1.46^{c} | 463.0 (M + H)⁺ | ¹H NMR (400 MHz, METHANOL-d4) δ |
| | (methyloxy)-3- | | | ppm 8.52 (d, J = 6.02 Hz, 1H), |
| | pyridinyl]phenyl}amino)-4- | | | 8.46 (br. s., 1H), 8.29 (s, 1H), |
| | pyrimidinyl]amino}benzenesulfonamide | | | 8.08-8.20 (m, 1H), 7.68-7.77 (m, 1H), |
| | | | | 7.62-7.68 (m, 1H), 7.39-7.58 (m, 4H), |
| | | | | 7.35 (d, J = 6.27 Hz, 1H), 7.26 (d, J = 7.53 Hz, |
| | | | | 1H), 6.25 (s, 1H), 4.02 (s, |
| | | | | 3H), 2.57 (s, 3H) |
| 341 | N-(3′-{[6-({3- | 1.45^{c} | 489.2 (M + H)⁺ | ¹H NMR (400 MHz, DMSO-d6) δ ppm |
| | [(methylamino)sulfonyl]phenyl}amino)- | | | 10.03 (s, 1H), 9.54 (s, 1H), 9.31 (s, |
| | 4-pyrimidinyl]amino}-4- | | | 1H), 8.34 (s, 1H), 8.09 (t, J = 1.88 Hz, |
| | biphenylyl)acetamide | | | 1H), 7.88-7.93 (m, 1H), 7.76 (s, 1H), |
| | | | | 7.67 (d, J = 8.53 Hz, 2H), 7.58 (d, J = 8.78 Hz, |
| | | | | 2H), 7.54 (d, J = 8.28 Hz, |
| | | | | 1H), 7.49 (t, J = 8.03 Hz, 1H), |
| | | | | 7.34-7.44 (m, 2H), 7.31 (d, J = 8.28 Hz, |
| | | | | 1H), 7.25 (d, J = 7.78 Hz, 1H), 6.23 (s, |
| | | | | 1H), 2.43 (d, J = 4.77 Hz, 3H), 2.06 (s, |
| | | | | 3H) |
| 342 | N-methyl-3-{[6-({4′- | 1.48^{c} | 525.1 (M + H)⁺ | ¹H NMR (400 MHz, DMSO-d6) δ ppm |
| | [(methylsulfonyl)amino]-3- | | | 9.87 (br. s., 1H), 9.56 (s, 1H), 9.34 (s, |
| | biphenylyl}amino)-4- | | | 1H), 8.35 (s, 1H), 8.10 (s, 1H), |
| | pyrimidinyl]amino}benzenesulfonamide | | | 7.93 (d, J = 8.03 Hz, 1H), 7.79 (s, 1H), |
| | | | | 7.62 (d, J = 8.28 Hz, 2H), 7.55 (d, J = 7.53 Hz, |
| | | | | 1H), 7.50 (t, J = 8.03 Hz, 1H), |
| | | | | 7.36-7.45 (m, 2H), 7.22-7.36 (m, |
| | | | | 4H), 6.24 (s, 1H), 3.03 (s, 3H), |
| | | | | 2.44 (d, J = 4.77 Hz, 3H) |
| 343 | N-(3′-{[6-({3- | 1.50^{c} | 489.0 (M + H)⁺ | ¹H NMR (400 MHz, DMSO-d6) δ ppm |
| | [(methylamino)sulfonyl]phenyl}amino)- | | | 10.03 (br. s., 1H), 9.54 (s, 1H), |
| | 4-pyrimidinyl]amino}-3- | | | 9.37 (s, 1H), 8.36 (s, 1H), 8.10 (br. s., 1H), |
| | biphenylyl)acetamide | | | 7.92 (br. s., 2H), 7.78 (br. s., 1H), |
| | | | | 7.61 (d, J = 7.78 Hz, 1H), 7.56 (d, J = 8.03 Hz, |
| | | | | 1H), 7.50 (t, J = 7.91 Hz, 1H), |
| | | | | 7.36-7.45 (m, 3H), 7.29-7.36 (m, |
| | | | | 2H), 7.22 (d, J = 7.78 Hz, 1H), 6.24 (s, |
| | | | | 1H), 2.45 (d, J = 5.02 Hz, 3H), 2.08 (s, |
| | | | | 3H) |
| 344 | N-methyl-3′-{[6-({3- | 1.52^{c} | 525.0 (M + H)⁺ | ¹H NMR (400 MHz, DMSO-d6) δ ppm |
| | [(methylamino)sulfonyl]phenyl}amino)- | | | 9.57 (s, 1H), 9.41 (s, 1H), 8.37 (s, |
| | 4-pyrimidinyl]amino}-4- | | | 1H), 8.10 (s, 1H), 7.93 (br. s., 3H), |
| | biphenylsulfonamide | | | 7.88 (s, 3H), 7.66 (d, J = 7.53 Hz, 1H), |
| | | | | 7.47-7.55 (m, 2H), 7.40-7.47 (m, |
| | | | | 2H), 7.30-7.40 (m, 2H), 6.25 (s, 1H), |
| | | | | 2.46 (dd, J = 5.02, 7.03 Hz, 6H) |
| 345 | N-methyl-3′-{[6-({3- | 1.53^{c} | 525.0 (M + H)⁺ | ¹H NMR (400 MHz, METHANOL-d4) δ |
| | [(methylamino)sulfonyl]phenyl}amino)- | | | ppm 8.31 (s, 1H), 8.12-8.16 (m, 1H), |
| | 4-pyrimidinyl]amino}-3- | | | 8.08-8.12 (m, 1H), 7.90-7.96 (m, |
| | biphenylsulfonamide | | | 1H), 7.82-7.88 (m, 1H), |
| | | | | 7.78-7.82 (m, 1H), 7.64-7.76 (m, 2H), |
| | | | | 7.44-7.56 (m, 4H), 7.37-7.43 (m, 1H), |
| | | | | 6.25 (s, 1H), 2.58-2.61 (m, 3H), |
| | | | | 2.55-2.58 (m, 3H) |
| 346 | 3-[(6-{[4-chloro-3-(3- | 1.55^{c} | 466.9 (M + H)⁺ | ¹H NMR (400 MHz, DMSO-d6) δ ppm |
| | pyridinyl)phenyl]amino}-4- | | | 9.58 (s, 1H), 9.50 (s, 1H), |
| | pyrimidinyl)amino]-N- | | | 8.60-8.68 (m, 2H), 8.35 (s, 1H), 8.07 (s, 1H), |
| | methylbenzenesulfonamide | | | 7.86-7.94 (m, 2H), 7.69-7.75 (m, |
| | | | | 2H), 7.47-7.56 (m, 3H), 7.41 (q, J = 4.94 Hz, |
| | | | | 1H), 7.33 (d, J = 8.03 Hz, |
| | | | | 1H), 6.21 (s, 1H), 2.44 (d, J = 4.77 Hz, |
| | | | | 3H) |
| 347 | 2′-chloro-5′-{[6-({3- | 1.50^{c} | 509.0 (M + H)⁺ | ¹H NMR (400 MHz, DMSO-d6) δ ppm |
| | [(methylamino)sulfonyl]phenyl}amino)- | | | 9.57 (s, 1H), 9.47 (s, 1H), 8.35 (s, |
| | 4-pyrimidinyl]amino}-3- | | | 1H), 8.02-8.10 (m, 2H), |
| | biphenylcarboxamide | | | 7.87-7.97 (m, 3H), 7.67-7.75 (m, 2H), |
| | | | | 7.55-7.63 (m, 2H), 7.47-7.55 (m, 2H), |
| | | | | 7.36-7.44 (m, 2H), 7.33 (d, J = 7.53 Hz, |
| | | | | 1H), 6.20 (s, 1H), 2.44 (d, J = 4.77 Hz, |
| | | | | 3H) |
| 348 | 3-[(6-{[6-chloro-3′-(4- | 1.69^{c} | 551.0 (M + H)⁺ | ¹H NMR (400 MHz, DMSO-d6) δ ppm |
| | morpholinyl)-3- | | | 9.56 (s, 1H), 9.42 (s, 1H), 8.34 (s, |
| | biphenylyl]amino}-4- | | | 1H), 8.08 (s, 1H), 7.87-7.95 (m, 1H), |
| | pyrimidinyl)amino]-N- | | | 7.62-7.70 (m, 2H), 7.50 (t, J = 8.03 Hz, |
| | methylbenzenesulfonamide | | | 1H), 7.45 (d, J = 8.53 Hz, 1H), |
| | | | | 7.38-7.43 (m, 1H), 7.29-7.36 (m, |
| | | | | 2H), 6.94-7.03 (m, 2H), 6.87 (d, J = 7.28 Hz, |
| | | | | 1H), 6.20 (s, 1H), |
| | | | | 3.71-3.78 (m, 4H), 3.12-3.19 (m, 4H), 2.44 (d, |
| | | | | J = 5.02 Hz, 3H) |
| 349 | 4-{[6-({3- | 1.93^{a} | 400.0 (M + H)⁺ | ¹H NMR (400 MHz, DMSO-d6) δ ppm |
| | [(methylamino)sulfonyl]phenyl}amino)- | | | 10.31 (br. s., 1H), 10.23 (br. s., 1H), |
| | 4- | | | 8.48 (s, 1H), 8.08 (br. s., 1H), |
| | pyrimidinyl]amino}benzoic acid | | | 7.83-7.94 (m, 3H), 7.75 (d, J = 8.28 Hz, |
| | | | | 2H), 7.57 (t, J = 7.65 Hz, 2H), 7.43 (d, |
| | | | | J = 7.53 Hz, 1H), 6.49 (s, 1H), |
| | | | | 2.45 (br. s., 3H) |
| 350 | [(3-{[6-({3- | 5.05^{b} | 430.1 (M + H)⁺ | ¹H NMR (400 MHz, DMSO-d6) δ ppm |
| | [(methylamino)sulfonyl]phenyl}amino)- | | | 13.02 (br. s., 1H), 9.58 (s, 1H), |
| | pyrimidinyl]amino}phenyl)oxy]acetic | | | 9.29 (s, 1H), 8.35 (s, 1H), 8.10 (t, J = 1.76 Hz, |
| | acid | | | 1H), 7.89-7.94 (m, 1H), 7.51 (t, J = 7.91 Hz, |
| | | | | 1H), 7.44 (q, J = 5.02 Hz, |
| | | | | 1H), 7.33 (d, J = 8.03 Hz, 1H), 7.26 (s, |
| | | | | 1H), 7.21 (t, J = 8.16 Hz, 1H), 7.14 (d, |
| | | | | J = 8.78 Hz, 1H), 6.54 (dd, J = 1.76, |
| | | | | 8.03 Hz, 1H), 6.22 (s, 1H), 4.65 (s, |
| | | | | 2H), 2.45 (d, J = 5.02 Hz, 3H) |
| 351 | N,N-dimethyl-4-{[6-({3- | 1.36^{c} | 427.0 (M + H)⁺ | ¹H NMR (400 MHz, DMSO-d6) δ ppm |
| | [(methylamino)sulfonyl]phenyl}amino)- | | | 9.65 (s, 1H), 9.53 (s, 1H), 8.44 (s, |
| | 4- | | | 1H), 8.17 (s, 1H), 7.98 (d, J = 8.28 Hz, |
| | pyrimidinyl]amino}benzamide | | | 1H), 7.72 (d, J = 8.53 Hz, 2H), 7.58 (t, |
| | | | | J = 7.91 Hz, 1H), 7.36-7.52 (m, 4H), |
| | | | | 6.31 (s, 1H), 3.03 (s, 6H), 2.51 (d, J = 5.02 Hz, |
| | | | | 3H) |
| 352 | N,N-dimethyl-2-[(3-{[6-({3- | 5.15^{b} | 457.1 (M + H)⁺ | ¹H NMR (400 MHz, DMSO-d6) δ ppm |
| | [(methylamino)sulfonyl]phenyl}amino)- | | | 9.70 (br. s., 1H), 9.40 (br. s., 1H), |
| | 4- | | | 8.37 (s, 1H), 8.08 (s, 1H), 7.86-7.92 (m, |
| | pyrimidinyl]amino}phenyl)oxy]acetamide | | | 1H), 7.53 (t, J = 7.91 Hz, 1H), 7.45 (q, |
| | trifluoroacetate | | | J = 4.77 Hz, 1H), 7.36 (d, J = 7.28 Hz, |
| | | | | 1H), 7.18-7.26 (m, 2H), 7.09 (d, J = 8.03 Hz, |
| | | | | 1H), 6.57-6.62 (m, 1H), |
| | | | | 6.21 (s, 1H), 4.79 (s, 2H), 3.02 (s, |
| | | | | 3H), 2.86 (s, 3H), 2.44 (d, J = 5.02 Hz, |
| | | | | 3H) |
| 353 | N-(2-hydroxyethyl)-4-{[6-({3- | 0.96^{c} | 443.1 (M + H)⁺ | ¹H NMR (400 MHz, DMSO-d6) δ ppm |
| | [(methylamino)sulfonyl]phenyl}amino)- | | | 9.57 (s, 1H), 9.49 (s, 1H), 8.38 (s, |
| | 4- | | | 1H), 8.22 (t, J = 5.40 Hz, 1H), 8.09 (s, |
| | pyrimidinyl]amino}benzamide | | | 1H), 7.91 (d, J = 8.03 Hz, 1H), |
| | | | | 7.80 (d, J = 8.53 Hz, 2H), 7.67 (d, J = 8.78 Hz, |
| | | | | 2H), 7.51 (t, J = 7.91 Hz, 1H), |
| | | | | 7.39 (q, J = 4.43 Hz, 1H), 7.33 (d, J = 7.53 Hz, |
| | | | | 1H), 6.25 (s, 1H), 4.68 (t, J = 5.40 Hz, |
| | | | | 1H), 3.50 (q, J = 5.69 Hz, |
| | | | | 2H), 3.30-3.36 (m, 2H), 2.44 (d, J = 5.02 Hz, |
| | | | | 3H) |
| 354 | N-methyl-3-{[6-({4-[(4-methyl-1- | 0.86^{c} | 482.1 (M + H)⁺ | ¹H NMR (400 MHz, DMSO-d6) δ ppm |
| | piperazinyl)carbonyl]phenyl}amino)- | | | 9.60 (s, 1H), 9.49 (s, 1H), 8.38 (s, |
| | 4- | | | 1H), 8.09-8.13 (m, 1H), |
| | pyrimidinyl]amino}benzenesulfonamide | | | 7.89-7.95 (m, 1H), 7.67 (d, J = 8.53 Hz, 2H), |
| | | | | 7.52 (t, J = 7.91 Hz, 1H), 7.43 (q, J = 4.77 Hz, |
| | | | | 1H), 7.32-7.38 (m, 3H), |
| | | | | 6.25 (s, 1H), 3.50 (br. s., 4H), 2.45 (d, |
| | | | | J = 4.77 Hz, 3H), 2.32 (br. s., 4H), |
| | | | | 2.20 (s, 3H) |
| 355 | 4-{[6-({3- | 1.32^{c} | 496.1 (M + H)⁺ | ¹H NMR (400 MHz, DMSO-d6) δ ppm |
| | [(methylamino)sulfonyl]phenyl}amino)- | | | 9.60 (s, 1H), 9.52 (s, 1H), 8.39 (s, |
| | 4-pyrimidinyl]amino}-N-(1- | | | 1H), 8.08-8.11 (m, 1H), 8.04 (d, J = 7.53 Hz, |
| | methyl-4-piperidinyl)benzamide | | | 1H), 7.89-7.95 (m, 1H), |
| | | | | 7.80 (d, J = 8.78 Hz, 2H), 7.67 (d, J = 8.78 Hz, |
| | | | | 2H), 7.51 (t, J = 8.03 Hz, |
| | | | | 1H), 7.39-7.47 (m, 1H), 7.34 (d, J = 8.28 Hz, |
| | | | | 1H), 6.25 (s, 1H), |
| | | | | 3.65-3.78 (m, 1H), 2.71-2.82 (m, 2H), 2.44 (d, |
| | | | | J = 4.77 Hz, 3H), 2.16 (s, 3H), |
| | | | | 1.88-1.98 (m, 2H), 1.70-1.80 (m, 2H), |
| | | | | 1.51-1.64 (m, 2H) |
| 356 | N-methyl-3-[(6-{[4-(1- | 1.26^{c} | 468.0 (M + H)⁺ | ¹H NMR (400 MHz, DMSO-d6) δ ppm |
| | piperazinylcarbonyl)phenyl]amino}- | | | 9.58 (s, 1H), 9.46 (s, 1H), 8.36 (s, |
| | 4- | | | 1H), 8.09 (s, 1H), 7.90 (dd, J = 1.63, |
| | pyrimidinyl)amino]benzenesulfonamide | | | 8.16 Hz, 1H), 7.65 (d, J = 8.53 Hz, |
| | | | | 2H), 7.50 (t, J = 7.91 Hz, 1H), |
| | | | | 7.39-7.45 (m, 1H), 7.33 (d, J = 8.53 Hz, |
| | | | | 3H), 6.23 (s, 1H), 3.41 (br. s., 4H), |
| | | | | 2.69 (br. s., 4H), 2.43 (d, J = 4.77 Hz, |
| | | | | 3H) |
| 357 | N-methyl-3-[(6-{[4-({4-[2- | 1.35^{c} | 526.1 (M + H)⁺ | ¹H NMR (400 MHz, METHANOL-d4) δ |
| | (methyloxy)ethyl]-1- | | | ppm 8.29-8.35 (m, 1H), |
| | piperazinyl}carbonyl)phenyl]amino}- | | | 8.12-8.18 (m, 1H), 7.70-7.77 (m, 1H), |
| | 4- | | | 7.59-7.65 (m, 2H), 7.45-7.56 (m, 2H), |
| | pyrimidinyl)amino]benzenesulfonamide | | | 7.38-7.45 (m, 2H), 6.24-6.30 (m, |
| | | | | 1H), 3.75 (br. s., 2H), 3.49-3.70 (m, |
| | | | | 4H), 3.35-3.38 (m, 3H), |
| | | | | 2.51-2.69 (m, 9H) |
| 358 | 4-{[6-({3- | 1.36^{c} | 457.0 (M + H)⁺ | ¹H NMR (400 MHz, DMSO-d6) δ ppm |
| | [(methylamino)sulfonyl]phenyl}amino)- | | | 9.57 (s, 1H), 9.50 (s, 1H), 8.38 (s, |
| | 4-pyrimidinyl]amino}-N-[2- | | | 1H), 8.31 (t, J = 5.27 Hz, 1H), 8.08 (s, |
| | (methyloxy)ethyl]benzamide | | | 1H), 7.89-7.94 (m, 1H), 7.80 (d, J = 8.78 Hz, |
| | | | | 2H), 7.67 (d, J = 8.78 Hz, |
| | | | | 2H), 7.51 (t, J = 8.03 Hz, 1H), 7.39 (q, |
| | | | | J = 5.02 Hz, 1H), 7.33 (d, J = 7.78 Hz, |
| | | | | 1H), 6.25 (s, 1H), 3.36-3.48 (m, 4H), |
| | | | | 3.26 (s, 3H), 2.44 (d, J = 5.02 Hz, 3H) |
| 359 | 4-{[6-({3- | 1.06^{c} | 471.0 (M + H)⁺ | ¹H NMR (400 MHz, DMSO-d6) δ ppm |
| | [(methylamino)sulfonyl]phenyl}amino)- | | | 9.59 (s, 1H), 9.51 (s, 1H), 8.38 (s, |
| | 4-pyrimidinyl]amino}-N-[3- | | | 1H), 8.27 (t, J = 5.65 Hz, 1H), 8.09 (s, |
| | (methyloxy)propyl]benzamide | | | 1H), 7.91 (d, J = 8.03 Hz, 1H), |
| | | | | 7.78 (d, J = 8.78 Hz, 2H), 7.67 (d, J = 8.78 Hz, |
| | | | | 2H), 7.51 (t, J = 8.03 Hz, 1H), |
| | | | | 7.41 (q, J = 4.94 Hz, 1H), 7.33 (d, J = 7.78 Hz, |
| | | | | 1H), 6.24 (s, 1H), 3.36 (t, J = 6.40 Hz, |
| | | | | 2H), 3.25-3.30 (m, 2H), |
| | | | | 3.23 (s, 3H), 2.44 (d, J = 5.02 Hz, 3H), |
| | | | | 1.74 (t, 2H) |
| 360 | N-[2-(dimethylamino)ethyl]-4-{[6- | 1.33^{c} | 470.0 (M + H)⁺ | ¹H NMR (400 MHz, DMSO-d6) δ ppm |
| | ({3- | | | 9.59 (s, 1H), 9.53 (s, 1H), 8.38 (s, |
| | [(methylamino)sulfonyl]phenyl}amino)- | | | 1H), 8.25 (t, J = 4.77 Hz, 1H), 8.09 (s, |
| | 4- | | | 1H), 7.87-7.94 (m, 1H), 7.79 (d, J = 8.78 Hz, |
| | pyrimidinyl]amino}benzamide | | | 2H), 7.68 (d, J = 8.78 Hz, |
| | | | | 2H), 7.51 (t, J = 8.03 Hz, 1H), 7.40 (q, |
| | | | | J = 4.68 Hz, 1H), 7.33 (d, J = 7.78 Hz, |
| | | | | 1H), 6.26 (s, 1H), 3.39 (q, J = 6.36 Hz, |
| | | | | 2H), 2.57 (br. s., 2H), 2.44 (d, J = 4.77 Hz, |
| | | | | 3H), 2.31 (br. s., 6H) |
| 361 | N,N-diethyl-4-{[6-({3- | 1.12^{c} | 455.1 (M + H)⁺ | ¹H NMR (400 MHz, DMSO-d6) δ ppm |
| | [(methylamino)sulfonyl]phenyl}amino)- | | | 9.59 (s, 1H), 9.46 (s, 1H), 8.38 (s, |
| | 4- | | | 1H), 8.11 (s, 1H), 7.92 (d, J = 8.03 Hz, |
| | pyrimidinyl]amino}benzamide | | | 1H), 7.66 (d, J = 8.53 Hz, 2H), 7.52 (t, |
| | | | | J = 7.91 Hz, 1H), 7.43 (q, J = 4.77 Hz, |
| | | | | 1H), 7.27-7.37 (m, 3H), 6.24 (s, 1H), |
| | | | | 3.33 (s, 4H), 2.45 (d, J = 4.77 Hz, 3H), |
| | | | | 1.07-1.17 (m, 6H) |
| 362 | N-methyl-3-[(6-{[4-(1- | 1.05^{c} | 453.1 (M + H)⁺ | ¹H NMR (400 MHz, DMSO-d6) δ ppm |
| | pyrrolidinylcarbonyl)phenyl]amino}- | | | 9.62 (s, 1H), 9.52 (s, 1H), 8.38 (s, |
| | pyrimidinyl)amino]benzenesulfonamide | | | 1H), 8.12 (s, 1H), 7.87-7.95 (m, 1H), |
| | | | | 7.67 (d, J = 8.78 Hz, 2H), |
| | | | | 7.47-7.56 (m, 3H), 7.44 (q, J = 4.85 Hz, 1H), |
| | | | | 7.34 (d, J = 7.78 Hz, 1H), 6.27 (s, 1H), |
| | | | | 3.46 (t, J = 6.40 Hz, 4H), 2.45 (d, J = 4.77 Hz, |
| | | | | 3H), 1.83 (br. s., 4H) |
| 363 | 3-({6-[(4-{[(3S)-3- | 1.35^{c} | 496.0 (M + H)⁺ | ¹H NMR (400 MHz, DMSO-d6) δ ppm |
| | (dimethylamino)-1- | | | 9.61 (s, 1H), 9.51 (br. s., 1H), 8.38 (s, |
| | pyrrolidinyl]carbonyl}phenyl)amino]- | | | 1H), 8.11 (s, 1H), 7.92 (d, J = 8.03 Hz, |
| | 4-pyrimidinyl}amino)-N- | | | 1H), 7.67 (d, J = 6.53 Hz, 2H), |
| | methylbenzenesulfonamide | | | 7.47-7.56 (m, 3H), 7.44 (br. s., 1H), |
| | | | | 7.34 (d, J = 7.78 Hz, 1H), 6.25 (s, 1H), |
| | | | | 3.39-3.77 (m, 3H), 3.16-3.27 (m, 1H), |
| | | | | 2.56-2.78 (m, 1H), 2.45 (s, 3H), |
| | | | | 2.19 (br. s., 3H), 2.00-2.16 (m, 4H), |
| | | | | 1.63-1.81 (m, 1H) |
| 364 | N-methyl-3-{[6-({4-[(4- | 1.33^{c} | 496.1 (M + H)⁺ | ¹H NMR (400 MHz, DMSO-d6) δ ppm |
| | methylhexahydro-1H-1,4- | | | 9.60 (br. s., 1H), 9.47 (s, 1H), 8.37 (s, |
| | diazepin-1- | | | 1H), 8.11 (br. s., 1H), 7.91 (d, J = 7.53 Hz, |
| | yl)carbonyl]phenyl}amino)-4- | | | 1H), 7.65 (d, J = 8.28 Hz, 2H), |
| | pyrimidinyl]amino}benzenesulfonamide | | | 7.51 (t, J = 7.91 Hz, 1H), 7.43 (br. s., |
| | | | | 1H), 7.34 (d, J = 7.78 Hz, 3H), 6.24 (s, |
| | | | | 1H), 3.60 (br. s., 2H), 3.46 (br. s., 2H), |
| | | | | 2.62 (m, 4H), 2.45 (s, 3H), |
| | | | | 2.21-2.31 (m, 3H), 1.84 (br. s., 1H), 1.76 (br. s., |
| | | | | 1H) |
| 365 | N-methyl-3-[(6-{[4-(4- | 1.09^{c} | 485.1 (M + H)⁺ | ¹H NMR (400 MHz, DMSO-d6) δ ppm |
| | thiomorpholinylcarbonyl)phenyl]amino}- | | | 9.66 (s, 1H), 9.56 (s, 1H), 8.44 (s, |
| | 4- | | | 1H), 8.17 (s, 1H), 7.95-8.01 (m, 1H), |
| | pyrimidinyl)amino]benzenesulfonamide | | | 7.74 (d, J = 8.53 Hz, 2H), 7.58 (t, J = 8.03 Hz, |
| | | | | 1H), 7.49 (q, J = 4.77 Hz, |
| | | | | 1H), 7.37-7.46 (m, 3H), 6.31 (s, 1H), |
| | | | | 3.80 (br. s., 4H), 2.71 (br. s., 4H), |
| | | | | 2.51 (d, J = 5.02 Hz, 3H) |
| 366 | 3-{[6-({4-[(4,4-difluoro-1- | 1.47^{c} | 503.2 (M + H)⁺ | ¹H NMR (400 MHz, DMSO-d6) δ ppm |
| | piperidinyl)carbonyl]phenyl}amino)- | | | 9.60 (s, 1H), 9.51 (s, 1H), 8.38 (s, |
| | 4-pyrimidinyl]amino}-N- | | | 1H), 8.11 (s, 1H), 7.88-7.95 (m, 1H), |
| | methylbenzenesulfonamide | | | 7.69 (d, J = 8.53 Hz, 2H), 7.52 (t, J = 8.03 Hz, |
| | | | | 1H), 7.38-7.47 (m, 3H), |
| | | | | 7.34 (d, J = 7.78 Hz, 1H), 6.25 (s, 1H), |
| | | | | 3.61 (br. s., 4H), 2.45 (d, J = 5.02 Hz, |
| | | | | 3H), 1.96-2.12 (m, 4H) |
| 367 | 3-({6-[(4-{[(3R)-3- | 1.32^{c} | 496.0 (M + H)⁺ | ¹H NMR (400 MHz, DMSO-d6) δ ppm |
| | (dimethylamino)-1- | | | 9.51 (s, 1H), 9.42 (br. s., 1H), 8.29 (s, |
| | pyrrolidinyl]carbonyl}phenyl)amino]- | | | 1H), 8.01 (s, 1H), 7.79-7.86 (m, 1H), |
| | 4-pyrimidinyl}amino)-N- | | | 7.58 (d, J = 7.53 Hz, 2H), |
| | methylbenzenesulfonamide | | | 7.36-7.47 (m, 3H), 7.33 (q, J = 4.77 Hz, 1H), |
| | | | | 7.24 (d, J = 7.78 Hz, 1H), 6.17 (s, 1H), |
| | | | | 3.07-3.66 (m, 4H), 2.48-2.69 (m, |
| | | | | 1H), 2.35 (d, J = 4.77 Hz, 3H), |
| | | | | 2.10 (br. s., 3H), 1.87-2.06 (m, 4H), |
| | | | | 1.53-1.72 (m, 1H) |
| 368 | N-[2-(dimethylamino)ethyl]-N- | 1.31^{c} | 484.2 (M + H)⁺ | ¹H NMR (400 MHz, DMSO-d6) δ ppm |
| | methyl-4-{[6-({3- | | | 10.14 (br. s., 2H), 8.75 (t, J = 5.65 Hz, |
| | [(methylamino)sulfonyl]phenyl}amino)- | | | 1H), 8.48 (s, 1H), 8.03 (s, 1H), |
| | 4- | | | 7.81-7.93 (m, 3H), 7.67 (d, J = 8.53 Hz, |
| | pyrimidinyl]amino}benzamide | | | 2H), 7.57 (t, J = 7.91 Hz, 1H), |
| | | | | 7.48-7.54 (m, 1H), 7.44 (d, J = 7.78 Hz, |
| | | | | 1H), 6.37 (s, 1H), 3.92 (d, J = 5.52 Hz, |
| | | | | 2H), 2.42-2.48 (m, 3H) |
| 369 | N-[2-(dimethylamino)ethyl]-N- | 1.76^{a} | 530.2 (M + H)⁺ | ¹H NMR (400 MHz, DMSO-d6) δ ppm |
| | methyl-4-[(6-{[5- | | | 2.44 (d, J = 4.77 Hz, 3 H) 2.47 (s, 3H, |
| | [(methylamino)sulfonyl]-2- | | | obscured by solvent) 2.99 (s, 3 H) |
| | (methylthio)phenyl]amino}-4- | | | 3.17 (s, 3 H) 3.36 (d, J = 5.52 Hz, 2 H) |
| | pyrimidinyl)amino]benzamide | | | 3.70-3.81 (m, 2 H) 5.89 (s, 1 H) |
| | trifluoroacetate | | | 7.42-7.56 (m, 4 H) 7.62-7.70 (m, 4 H) |
| | | | | 8.31 (s, 1 H) 9.21-9.25 (m, 1 H) |
| | | | | 9.72 (s, 1 H) |
| 370 | N-[(4-{[6-({3- | 0.64^{c} | 457.1 (M + H)⁺ | ¹H NMR (400 MHz, DMSO-d6) δ ppm |
| | [(methylamino)sulfonyl]phenyl}amino)- | | | 9.92 (s, 1H), 9.59 (br. s., 1H), 8.40 (s, |
| | 4- | | | 1H), 8.04 (s, 1H), 7.81-7.88 (m, 1H), |
| | pyrimidinyl]amino}phenyl)carbonyl]glycine | | | 7.56 (t, J = 8.03 Hz, 1H), 7.48 (q, J = 4.85 Hz, |
| | | | | 1H), 7.41 (d, J = 8.03 Hz, |
| | | | | 1H), 7.15 (t, J = 8.03 Hz, 1H), 6.98 (s, |
| | | | | 1H), 6.89 (d, J = 8.03 Hz, 1H), |
| | | | | 6.52 (dd, J = 1.76, 8.03 Hz, 1H), 6.20 (s, |
| | | | | 1H), 2.44 (d, J = 4.77 Hz, 3H) |
| 371 | N-methyl-3-[(6-{[3-(6-oxo-1,6- | 1.31^{c} | 449.2 (M + H)⁺ | ¹H NMR (400 MHz, DMSO-d6) δ ppm |
| | dihydro-3- | | | 9.71 (br. s., 1H), 9.46 (br. s., 1H), |
| | pyridinyl)phenyl]amino}-4- | | | 8.35 (s, 1H), 8.13 (s, 1H), 7.93 (d, J = 8.03 Hz, |
| | pyrimidinyl)amino]benzenesulfonamide | | | 1H), 7.80 (dd, J = 2.76, 9.29 Hz, |
| | | | | 1H), 7.71 (br. s., 1H), 7.66 (d, J = 2.51 Hz, |
| | | | | 1H), 7.56 (d, J = 7.03 Hz, 1H), |
| | | | | 7.46-7.53 (m, 1H), 7.30-7.39 (m, |
| | | | | 4H), 7.17 (d, J = 7.78 Hz, 1H), |
| | | | | 6.46 (d, J = 9.54 Hz, 1H), 6.33 (s, 1H), |
| | | | | 2.42-2.47 (m, 3H) |
| 372 | 3-({6-[(3-hydroxyphenyl)amino]- | 4.99^{b} | 372.1 (M + H)⁺ | ¹H NMR (400 MHz, DMSO-d6) δ ppm |
| | 4-pyrimidinyl}amino)-N- | | | 9.92 (s, 1H), 9.59 (br. s., 1H), 8.40 (s, |
| | methylbenzenesulfonamide | | | 1H), 8.04 (s, 1H), 7.79-7.90 (m, 1H), |
| | trifluoroacetate | | | 7.56 (t, J = 8.03 Hz, 1H), 7.48 (q, J = 4.85 Hz, |
| | | | | 1H), 7.41 (d, J = 8.03 Hz, |
| | | | | 1H), 7.15 (t, J = 8.03 Hz, 1H), 6.98 (s, |
| | | | | 1H), 6.89 (d, J = 8.03 Hz, 1H), |
| | | | | 6.52 (dd, J = 1.76, 8.03 Hz, 1H), 6.20 (s, |
| | | | | 1H), 2.44 (d, J = 4.77 Hz, 3H) |
| 373 | N-methyl-4-(methylsulfonyl)-3- | 2.60^{a} | 502.0 (M + H)⁺ | ¹H NMR (400 MHz, DMSO-d6) δ ppm |
| | [(6-{[4- | | | 9.81 (s, 1 H), 9.00 (s, 1 H), |
| | trifluoromethyl)phenyl]amino}-4- | | | 8.42-8.39 (m, 2 H), 8.12 (d, J = 8.28 Hz, 1 |
| | pyrimidinyl)amino]benzene- | | | H), 7.86 (d, J = 8.53 Hz, 2 H), |
| | sulfonamide | | | 7.79-7.82 (m, 1 H), 7.64-7.71 (m, 3 H), |
| | | | | 6.39 (s, 1 H), 3.32 (s, 3 H), 2.50 (s, |
| | | | | 3H) |
| 374 | 3-({6-[(4-chlorophenyl)amino]-4- | 2.40^{a} | 468.0 (M + H)⁺ | ¹H NMR (400 MHz, DMSO-d₆) δ ppm |
| | pyrimidinyl}amino)-N-methyl-4- | | | 2.47 (d, 3H, obscured by solvent) |
| | (methylsulfonyl)benzenesulfonamide | | | 3.31 (s, 3 H) 6.30 (s, 1 H) 7.38 (d, J = 8.78 Hz, |
| | trifluoroacetate | | | 2 H) 7.63 (d, J = 8.78 Hz, 2 H) |
| | | | | 7.69 (dd, J = 8.41, 1.63 Hz, 1 H) 7.79 (q, |
| | | | | J = 4.77 Hz, 1 H) 8.12 (d, J = 8.28 Hz, 1 |
| | | | | H) 8.36 (s, 1 H) 8.42 (d, J = 1.51 Hz, 1 |
| | | | | H) 9.00 (br. s., 1 H) 9.60 (s, 1 H) |
| 375 | 3-(6-(4- | 1.11^{c} | 509.9 (M + H)⁺ | ¹H NMR (400 MHz, DMSO-d₆) δ ppm |
| | chlorophenylamino)pyrimidin-4- | | | 0.90 (d, J = 7.06 Hz, 6 H) |
| | ylamino)-4-(isobutylsulfonyl)-N- | | | 1.95-2.03 (m, 1 H) 2.47 (d, 3H, obscured by |
| | methylbenzenesulfonamide | | | solvent) 3.27 (d, J = 6.17 Hz, 2 H) |
| | trifluoroacetate | | | 6.37 (s, 1 H) 7.33 (d, J = 8.82 Hz, 2 H) |
| | | | | 7.62-7.67 (m, 3 H) 8.06 (d, J = 8.38 Hz, 1 |
| | | | | H) 8.31 (s, 1 H) 8.36 (d, J = 1.76 Hz, 1 |
| | | | | H) 9.67 (s, 1 H) |
| 376 | 3-(6-(4- | 1.19^{c} | 482.0 (M + H)⁺ | ¹H NMR (400 MHz, DMSO-d₆) δ ppm |
| | chlorophenylamino)pyrimidin-4- | | | 1.08 (t, J = 7.50 Hz, 3 H) 2.47 (d, 3H, |
| | ylamino)-4-(ethylsulfonyl)-N- | | | obscured by solvent) 3.33 (q, 2H, |
| | methylbenzenesulfonamide | | | obscured by solvent) 6.33 (s, 1 H) |
| | trifluoroacetate | | | 7.32 (s, 2 H) 7.58-7.67 (m, 3 H) |
| | | | | 7.81 (q, J = 4.85 Hz, 1 H) 8.04 (d, J = 8.38 Hz, |
| | | | | 1 H) 8.32 (s, 1 H) 8.43 (d, J = 1.76 Hz, |
| | | | | 1 H) 8.90 (s, 1 H) 9.61 (s, 1 H) |
| 377 | 3-({6-[(4-chlorophenyl)amino]-4- | 2.33^{a} | 502.0 (M + H)⁺ | ¹H NMR (400 MHz, DMSO-d₆) δ ppm |
| | pyrimidinyl}amino)-N-methyl-4- | | | 1.45 (d, J = 6.27 Hz, 3 H) 2.44 (d, |
| | [(2,2,2-trifluoro-1- | | | J = 4.52 Hz, 3 H) 5.32-5.44 (m, 1 H) |
| | methylethyl)oxy]benzenesulfonamide | | | 6.14 (s, 1 H) 7.33 (d, J = 8.53 Hz, 2 H) |
| | | | | 7.37-7.43 (m, 1 H) 7.44-7.52 (m, 2 |
| | | | | H) 7.63 (d, J = 8.78 Hz, 2 H) 8.21 (d, |
| | | | | J = 1.51 Hz, 1 H) 8.26 (s, 1 H) 8.59 (br. |
| | | | | s., 1 H) 9.32 (s, 1 H) |
| 378 | 3-({6-[(4-chlorophenyl)amino]-4- | 2.32^{a} | 502.0 (M + H)⁺ | ¹H NMR (400 MHz, DMSO-d₆) δ ppm |
| | pyrimidinyl}amino)-N-methyl-4- | | | 1.45 (d, J = 6.53 Hz, 3 H) 2.44 (d, |
| | [(2,2,2-trifluoro-1- | | | J = 4.77 Hz, 3 H) 5.33-5.44 (m, 1 H) |
| | methylethyl)oxy]benzenesulfonamide | | | 6.11-6.15 (m, 1 H) 7.33 (d, 2 H) |
| | | | | 7.37-7.43 (m, 1 H) 7.44-7.52 (m, 2 H) |
| | | | | 7.63 (d, J = 8.78 Hz, 2 H) 8.21 (d, |
| | | | | J = 2.26 Hz, 1 H) 8.26 (s, 1 H) 8.59 (s, |
| | | | | 1 H) 9.32 (s, 1 H) |
| 379 | 3-({6-[(5-chloro-2- | 2.15^{a} | 502.9 (M + H)⁺ | ¹H NMR (400 MHz, DMSO-d₆) δ ppm |
| | pyridinyl)amino]-4- | | | 1.44 (d, J = 6.27 Hz, 3 H) 2.44 (d, 3 H) |
| | pyrimidinyl}amino)-N-methyl-4- | | | 5.37-5.45 (m, 1 H) 7.20 (s, 1 H) |
| | [(2,2,2-trifluoro-1- | | | 7.37-7.43 (m, 1 H) 7.46 (d, J = 8.78 Hz, 1 |
| | methylethyl)oxy]benzenesulfonamide | | | H) 7.52 (dd, J = 8.53, 2.01 Hz, 1 H) |
| | | | | 7.62 (d, J = 9.03 Hz, 1 H) 7.81 (dd, |
| | | | | J = 9.03, 2.76 Hz, 1 H) 8.13 (d, J = 2.26 Hz, |
| | | | | 1 H) 8.26 (d, J = 2.26 Hz, 1 H) |
| | | | | 8.28 (s, 1 H) 8.77 (s, 1 H) 10.03 (s, 1 H) |
| 380 | 3-({6-[(5-chloro-2- | 2.15^{a} | 502.9 (M + H)⁺ | ¹H NMR (400 MHz, DMSO-d₆) δ ppm |
| | pyridinyl)amino]-4- | | | 1.44 (d, J = 6.27 Hz, 3 H) 2.44 (br. s., 3 |
| | pyrimidinyl}amino)-N-methyl-4- | | | H) 5.35-5.46 (m, 1 H) 7.20 (s, 1 H) |
| | [(2,2,2-trifluoro-1- | | | 7.40 (br. s., 1 H) 7.46 (d, J = 8.78 Hz, 1 |
| | methylethyl)oxy]benzenesulfonamide | | | H) 7.52 (dd, J = 8.78, 2.26 Hz, 1 H) |
| | | | | 7.62 (d, J = 9.03 Hz, 1 H) 7.81 (dd, |
| | | | | J = 8.91, 2.64 Hz, 1 H) 8.13 (d, J = 2.26 Hz, |
| | | | | 1 H) 8.26 (d, J = 2.26 Hz, 1 H) |
| | | | | 8.28 (s, 1 H) 8.77 (s, 1 H) 10.02 (s, 1 H) |

^{a}LCMS Method: Agilent 1100 Series LC/MSD SL or VL using electrospray positive [ES+ve to give M + H⁺] equipped with a Sunfire C18 5.0 μm column (3.0 mm × 50 mm, i.d.), eluting with 0.05% TFA in water (solvent A) and 0.05% TFA in CH₃CN (solvent B), using the following elution gradient: 10-100% (solvent B) over 2.5 min and holding at 100% for 1.7 min at a flow rate of 1.0 mL/min.
^{b}LCMS Method: Agilent 1100 Series LC/MSD SL or VL using electrospray positive [ES+ve to give M + H⁺] equipped with a Sunfire C18 5.0 μm column (3.0 mm × 50 mm, i.d.), eluting with 0.05% TFA in water (solvent A) and 0.05% TFA in CH₃CN (solvent B), using the following elution gradient 10-100% (solvent B) over 10.0 min and holding at 100% for 1.7 min at a flow rate of 1.0 mL/min.
^{c}LCMS Method: Agilent 1200 Series LC/MSD SL or VL using electrospray positive [ES+ve to give M + H⁺] equipped with a XBridge C18 3.5 μm column (50 × 4.6 mm, i.d.), eluting with 10 mM NH₄HCO₃ in water (solvent A) and CH₃CN (solvent B), using the following elution gradient 5-95% (solvent B) over 1.2 min and holding at 95% for 1.5 min at a flow rate of 2.0 mL/min.
^{d}LCMS Method: Agilent 1200 Series LC/MSD VL using electrospray positive [ES+ve to give M + H⁺] equipped with a shim-pack XR-ODS 2.2 μm column (3.0 mm × 30 mm, 3.0 mm i.d.) eluting with 0.0375% TFA in water (solvent A) and 0.01875% TFA in CH₃CN (solvent B), using the following elution gradient 10-80% (solvent B) over 0.9 min and holding at 80% for 0.6 min at a flow rate of 1.2 mL/min.

### Pharmaceutical Compositions

### Example A

Tablets are prepared using conventional methods and are formulated as follows:

| Ingredient | Amount per tablet |
|---|---|
| Compound of Example I | 5 mg |
| Microcrystalline cellulose | 100 mg |
| Lactose | 100 mg |
| Sodium starch glycollate | 30 mg |
| Magnesium stearate | 2 mg |
| Total | 237 mg |

### Example B

Capsules are prepared using conventional methods and are formulated as follows:

| Ingredient | Amount per tablet |
|---|---|
| Compound of Example 3 | 15 mg |
| Dried starch | 178 mg |
| Magnesium stearate | 2 mg |
| Total | 195 mg |

### Biological Assay(s)

Materials: His-MBP-TEV-Full length human TNNI3K (hTNNI3K) was expressed in Baculokinase system and purified from amylase affinity column followed by Superdex200. The fluorescent ligand 5-({[2-({[3-({-4-[(5-hydroxy-2-methylphenyl)amino]-2-pyrimidinyl}amino)phenyl]carbonyl}amino)ethyl]amino}carbonyl)-2-(6-hydroxy-3-oxo-3H-xanthen-9-yl)benzoic acid was used. The preparation of this fluorescent ligand is disclosed in U.S. Provisional Patent Application No. 61/237,815 filed Aug. 28, 2009, the disclosure of which is incorporated by reference herein. The other buffer components, including MgCl₂ (Catalog Number M1028), Bis-Tris (Catalog Number B7535), DTT (Catalog Number D9779) and Chaps (Catalog Number C3023) were purchased from Sigma-Aldrich.

### Biological Assay Method I:

A fluorescent polarization assay was used to determine does response of compound inhibition on hTNNI3K ATP binding. The binding of 5-({[2-({[3-({4-[(5-hydroxy-2-methylphenyl)amino]-2-pyrimidinyl}amino)phenyl]carbonyl}amino)ethyl]amino}carbonyl)-2-(6-hydroxy-3-oxo-3H-xanthen-9-yl)benzoic acid to the hTNNI3K ATP binding pocket results in increase of fluorescent polarization and the displacement of 5-({[2-({[3-({4-[(5-hydroxy-2-methylphenyl)amino]-2-pyrimidinyl}amino)phenyl]carbonyl}amino)ethyl]amino}carbonyl)-2-(6-hydroxy-3-oxo-3H-xanthen-9-yl)benzoic acid by a competitive compound leads to fluorescent polarization decrease.

Solution 1: Ten (10) mL of a 5 nM 5-({[2-({[3-({4-[(5-hydroxy-2-methylphenyl)amino]-2-pyrimidinyl}amino)phenyl]carbonyl}amino)ethyl]amino}carbonyl)-2-(6-hydroxy-3-oxo-3H-xanthen-9-yl)benzoic acid solution (Solution 1) was prepared by mixing 5 μL of 1 M DTT and 80 μL of 10% (w/v) Chaps and 5 μL of a 10 μM 5-({[2-({[3-({4-[(5-hydroxy-2-methylphenyl)amino]-2-pyrimidinyl}amino)phenyl]carbonyl}amino)ethyl]amino}carbonyl)-2-(6-hydroxy-3-oxo-3H-xanthen-9-yl)benzoic acid stock solution into 9910 μL buffer (20 mM Tris, 15 mM MgCl₂, pH 7.5). (Stock solution: 10 μM solution of 5-({[2-({[3-({4-[(5-hydroxy-2-methylphenyl)amino]-2-pyrimidinyl}amino)phenyl]carbonyl}amino) ethyl]amino}carbonyl)-2-(6-hydroxy-3-oxo-3H-xanthen-9-yl)benzoic acid in 100% DMSO)

Solution 2 was formed by mixing 53.8 μL of 2.6 μM hTNNI3K with a 6946.2 μL aliquot of Solution 1 (the above 5-({[2-({[3-({4-[(5-hydroxy-2-methylphenyl)amino]-2-pyrimidinyl}amino)phenyl]carbonyl}amino)ethyl]amino}carbonyl)-2-(6-hydroxy-3-oxo-3H-xanthen-9-yl)benzoic acid solution) to make up a 7 mL of mixture of hTNNI3K and 5-({[2-({[3-({4-[(5-hydroxy-2-methylphenyl)amino]-2-pyrimidinyl}amino)phenyl]carbonyl}amino)ethyl]amino}carbonyl)-2-(6-hydroxy-3-oxo-3H-xanthen-9-yl)benzoic acid (Solution 2).

Fifty (50) nL of inhibitors in DMSO (or DMSO controls) were stamped into a 384-well low volume Greiner black plate, followed by addition of 5 μL of Solution 1 to column 18 and 5 μL Solution 2 to columns 1-17 and 19-24 of the plate. The plate was then spun at 500 rpm for 30 seconds and incubated at rt for 60 min. After that, the fluorescent polarization was measured on Analyst (ex/em: 485/530 nm, Dichroic: 505). For dose response experiments, normalized data were fit by ABASE/XC₅₀ and pXC₅₀=(log((b−y)/(y−a)))/d−log(x), where x is the compound concentration and y is the % activity at specified compound concentration, a is the minimum % activity, b is the maximum % activity, and d is the Hill slope.

The pXC₅₀s are averaged to determine a mean value, for a minimum of 2 experiments. As determined using the above method, the compounds of Examples 1-380 exhibited a pXC₅₀ greater than or equal to approximately 6.0. For instance, the compounds of Example 55 and Example 284 each inhibited hTNNI3K in the above method with a mean pXC₅₀ of approximately 7.0.

1. A compound according to Formula I: 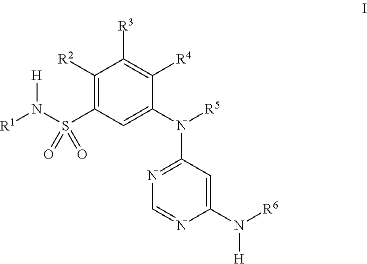
wherein:
R¹ is (C₁-C₄)alkyl;
R² is hydrogen or halogen;
R³ is hydrogen, halogen, (C₁-C₄)alkyl, (C₁-C₄)haloalkyl, (C₃-C₆)cycloalkyl, aryl, hydroxyl, hydroxy(C₁-C₄)alkyl-, (C₁-C₄)alkoxy, (C₁-C₄)alkoxy(C₁-C₄)alkyl-, (C₁-C₄)haloalkoxy, (C₃-C₆)cycloalkyloxy, (C₁-C₄)alkylthio-, amino, (C₁-C₄)alkylamino, or ((C₁-C₄)alkyl)((C₁-C₄)alkyl)amino;
R⁴ is hydrogen, halogen, (C₁-C₈)alkyl, (C₁-C₈)haloalkyl, (C₃-C₈)cycloalkyl, hydroxyl, hydroxy(C₁-C₈)alkyl-, (C₁-C₈)alkoxy, (C₁-C₄)alkoxy(C₁-C₈)alkyl-, (C₁-C₈)haloalkoxy, (C₃-C₈)cycloalkyloxy, (C₁-C₈)alkylthio-, (C₁-C₈)haloalkylthio-, —SO₂(C₁-C₄)alkyl, amino, —NHR⁷, or —NR⁷R⁸;
R⁵ is hydrogen;
or R⁴ and R⁵ taken together with atoms through which they are connected form a 5 or 6 membered ring, optionally containing one or two additional heteroatoms selected from N, O and S, which ring may be unsubstituted or substituted with one to three substituents independently selected from (C₁-C₄)alkyl, (C₁-C₄)haloalkyl, hydroxy(C₁-C₄)alkyl-, oxo, hydroxyl, (C₁-C₄)alkoxy, (C₁-C₄)haloalkoxy, and (C₁-C₄)alkylthio-;
R⁶ is (C₁-C₈)alkyl, (C₂-C₈)alkenyl, (C₂-C₈)alkynyl, (C₃-C₈)cycloalkyl, aryl, or heteroaryl, wherein any aryl or heteroaryl group is optionally substituted one to three times, independently, by halogen, (C₁-C₆)alkyl, (C₂-C₆)alkenyl, (C₂-C₆)alkynyl, (C₃-C₆)cycloalkyl, (C₁-C₄)haloalkyl, cyano, —CO(C₁-C₄)alkyl, —CO₂H, —CO₂R⁷, —CONH₂, —CONHR⁷, —CONR⁷R⁸, HO₂C(C₁-C₂)alkyl-, R⁷O₂C(C₁-C₂)alkyl-, —SR″, —SO₂(C₁-C₄)alkyl, —SO₂NH₂, —SO₂NHR⁷, —SO₂NR⁷R⁸, nitro, amino, —NHR⁷, —NR⁷R⁸, amino(C₁-C₂)alkyl-, R⁷HN(C₁-C₂)alkyl-, R⁷R⁸N(C₁-C₂)alkyl-, —NHCO(C₁-C₄)alkyl, —NHSO₂(C₁-C₄)alkyl, oxo, hydroxyl, —OR⁷, hydroxy(C₁-C₂)alkyl-, R⁷O(C₁-C₂)alkyl-, cyano(C₁-C₂)alkyl-, aryl, heteroaryl, or heteroaryl(C₁-C₂)alkyl-, wherein any said aryl or heteroaryl is optionally substituted one to three times, independently, by halogen, (C₁-C₆)alkyl, (C₃-C₆)cycloalkyl, (C₁-C₄)haloalkyl, cyano, —CO(C₁-C₄)alkyl, —CO₂H, —CO₂R⁷, —CONH₂, —CONHR⁷, —CONR⁷R⁸, —SR⁷, —SO₂(C₁-C₄)alkyl, —SO₂NH₂, —SO₂NHR⁷, —SO₂NR⁷R⁸, nitro, amino, —NHR⁷, —NR⁷R⁸, —NHCO(C₁-C₄)alkyl, —NHSO₂(C₁-C₄)alkyl, oxo, hydroxyl, —OR⁷, hydroxy(C₁-C₂)alkyl-, or R⁷O(C₁-C₂)alkyl-;
R⁷ is (C₁-C₄)alkyl, aryl, heterocycloalkyl, or heterocycloalkyl(C₁-C₂)alkyl, wherein said (C₁-C₄)alkyl is optionally substituted one to three times, independently, by halogen, hydroxyl, (C₁-C₄)alkoxy, amino, (C₁-C₄)alkylamino, ((C₁-C₄)alkyl)((C₁-C₄)alkyl)amino, —CO₂H, —CO₂(C₁-C₄)alkyl, —CONH₂, —CONH(C₁-C₄)alkyl, or —CON((C₁-C₄)alkyl)((C₁-C₄)alkyl); and wherein any heterocycloalkyl is optionally substituted by (C₁-C₄)alkyl; and
R⁸ is (C₁-C₄)alkyl;
or R⁷ and R⁸ taken together with the nitrogen to which they are attached represent a 5-7 membered heterocyclic ring, optionally containing an additional heteroatom selected from oxygen, nitrogen, and sulfur, wherein said ring is optionally substituted one or two times, independently, by halogen, (C₁-C₄)alkyl, (C₁-C₄)haloalkyl, amino, (C₁-C₄)alkylamino, ((C₁-C₄)alkyl)((C₁-C₄)alkyl)amino, hydroxyl, oxo, (C₁-C₄)alkoxy, or (C₁-C₄)alkoxy(C₁-C₄)alkyl;
or a salt thereof.

2. The compound or salt according to claim 1, wherein R¹ is methyl.

3. The compound or salt according to claim 1, wherein R² and R³ are each hydrogen.

4. The compound or salt according to claim 1, wherein R⁴ is hydrogen, halogen, (C₁-C₈)alkyl, (C₁-C₈)haloalkyl, (C₃-C₈)cycloalkyl, hydroxyl, hydroxy(C₁-C₈)alkyl-, (C₁-C₈)alkoxy, (C₁-C₄)alkoxy(C₁-C₈)alkyl-, (C₁-C₈)haloalkoxy, (C₃-C₈)cycloalkyloxy, (C₁-C₈)alkylthio-, (C₁-C₈)haloalkylthio-, —SO₂(C₁-C₄)alkyl, amino, (C₁-C₄)alkylamino, (C₁-C₄)haloalkylamino, ((C₁-C₄)alkyl)((C₁-C₄)alkyl)amino, ((C₁-C₄)alkyl)((C₁-C₄)haloalkyl)amino, ((C₁-C₄)haloalkyl)((C₁-C₄)haloalkyl)amino, pyrrolidinyl, imidazolidinyl, pyrazolidinyl, piperidinyl, piperazinyl, morpholinyl, or thiomorpholinyl, wherein said pyrrolidinyl, imidazolidinyl, pyrazolidinyl, piperidinyl, piperazinyl, morpholinyl, or thiomorpholinyl is optionally substituted one or two times, independently, by halogen, (C₁-C₄)alkyl, (C₁-C₄)haloalkyl, amino, (C₁-C₄)alkylamino, ((C₁-C₄)alkyl)((C₁-C₄)alkyl)amino, hydroxyl, oxo, (C₁-C₄)alkoxy, or (C₁-C₄)alkoxy(C₁-C₄)alkyl.

5. The compound or salt according to claim 1, wherein R⁴ and R⁵ taken together represent —CH₂CH₂—.

6. The compound or salt according to claim 1, wherein R⁶ is (C₁-C₆)alkyl, phenyl, dihydroindenyl, tetrahydronaphthalenyl, oxazolyl, thiazolyl, thiadiazolyl, pyridinyl, pyrimidinyl, indolyl, indazolyl, dihydroindolyl, dihydroisoindolyl, chromenyl, dihydrobenzimidazolyl, dihydrobenzoxazolyl, benzothiazolyl, dihydrobenzoisothiazolyl, quinolinyl, isoquinolinyl, dihydroquinolinyl, tetrahydroquinolinyl, tetrahydroisoquinolinyl, benzodioxolyl, or dihydrobenzodioxinyl, wherein said phenyl, dihydroindenyl, tetrahydronaphthalenyl, oxazolyl, thiazolyl, thiadiazolyl, pyridinyl, pyrimidinyl, indolyl, indazolyl, dihydroindolyl, dihydroisoindolyl, chromenyl, dihydrobenzimidazolyl, dihydrobenzoxazolyl, benzothiazolyl, dihydrobenzoisothiazolyl, quinolinyl, isoquinolinyl, dihydroquinolinyl, tetrahydroquinolinyl, tetrahydroisoquinolinyl, benzodioxolyl, or dihydrobenzodioxinyl group is optionally substituted one to three times, independently, by halogen, (C₁-C₆)alkyl, (C₂-C₆)alkenyl, (C₂-C₆)alkynyl, (C₃-C₆)cycloalkyl, (C₁-C₄)haloalkyl, cyano, —CO(C₁-C₄)alkyl, —CO₂H, —CO₂R⁷, —CONH₂, —CONHR⁷, —CONR⁷R⁸, HO₂C(C₁-C₂)alkyl-, R⁷O₂C(C₁-C₂)alkyl-, cyano(C₁-C₂)alkyl-, —SR⁷, —SO₂(C₁-C₄)alkyl, —SO₂NH₂, —SO₂NHR⁷, —SO₂NR⁷R⁸, nitro, amino, —NHR⁷, —NR⁷R⁸, amino(C₁-C₂)alkyl-, R⁷R⁸N(C₁-C₂)alkyl-, R⁷R⁸N(C₁-C₂)alkyl-, triazolyl(C₁-C₂)alkyl-, —NHCO(C₁-C₄)alkyl, —NHSO₂(C₁-C₄)alkyl, oxo, hydroxyl, —OR⁷, hydroxy(C₁-C₂)alkyl-, R⁷O(C₁-C₂)alkyl-, phenyl, thienyl, pyrazolyl, imidazolyl, oxazolyl, thiazolyl, or pyridinyl, wherein said phenyl, thienyl, pyrazolyl, imidazolyl, oxazolyl, thiazolyl, or pyridinyl is optionally substituted one or two times, independently, by halogen, (C₁-C₆)alkyl, (C₃-C₆)cycloalkyl, (C₁-C₄)haloalkyl, cyano, —CO(C₁-C₄)alkyl, —CO₂H, —CO₂R⁷, —CONH₂, —CONHR⁷, —CONR⁷R⁸, —SR⁷, —SO₂(C₁-C₄)alkyl, —SO₂NH₂, —SO₂NHR⁷, —SO₂NR⁷R⁸, nitro, amino, —NHR⁷, —NR⁷R⁸, —NHCO(C₁-C₄)alkyl, —NHSO₂(C₁-C₄)alkyl, oxo, hydroxyl, —OR⁷, hydroxy(C₁-C₂)alkyl-, or R⁷O(C₁-C₂)alkyl-.

7. The compound or salt according to claim 1, wherein R⁶ is phenyl optionally substituted one to three times, independently, by halogen, (C₁-C₆)alkyl, (C₂-C₆)alkenyl, (C₂-C₆)alkynyl, (C₃-C₆)cycloalkyl, (C₁-C₄)haloalkyl, cyano, —CO(C₁-C₄)alkyl, —CO₂H, —CO₂R⁷, —CONH₂, —CONHR⁷, —CONR⁷R⁸, HO₂C(C₁-C₂)alkyl-, R⁷O₂C(C₁-C₂)alkyl-, cyano(C₁-C₂)alkyl-, —SR⁷, —SO₂(C₁-C₄)alkyl, —SO₂NH₂, —SO₂NHR⁷, —SO₂NR⁷R⁸, nitro, amino, —NHR⁷, —NR⁷R⁸, amino(C₁-C₂)alkyl-, R⁷HN(C₁-C₂)alkyl-, R⁷R⁸N(C₁-C₂)alkyl-, triazolyl(C₁-C₂)alkyl-, —NHCO(C₁-C₄)alkyl, —NHSO₂(C₁-C₄)alkyl, oxo, hydroxyl, —OR⁷, hydroxy(C₁-C₂)alkyl-, R⁷O(C₁-C₂)alkyl-, phenyl, thienyl, pyrazolyl, imidazolyl, oxazolyl, thiazolyl, or pyridinyl, wherein said phenyl, thienyl, pyrazolyl, imidazolyl, oxazolyl, thiazolyl, or pyridinyl is optionally substituted one or two times, independently, by halogen, (C₁-C₆)alkyl, (C₃-C₆)cycloalkyl, (C₁-C₄)haloalkyl, cyano, —CO(C₁-C₄)alkyl, —CO₂H, —CO₂R⁷, —CONH₂, —CONHR⁷, —CONR⁷R⁸, —SR⁷, —SO₂(C₁-C₄)alkyl, —SO₂NH₂, —SO₂NHR⁷, —SO₂NR⁷R⁸, nitro, amino, —NHR⁷, —NR⁷R⁸, —NHCO(C₁-C₄)alkyl, —NHSO₂(C₁-C₄)alkyl, oxo, hydroxyl, —OR⁷, hydroxy(C₁-C₂)alkyl-, or R⁷O(C₁-C₂)alkyl-.

8. The compound or salt according to claim 1, wherein R⁶ is pyridinyl optionally substituted one or two times, independently, by halogen, (C₁-C₆)alkyl, (C₃-C₆)cycloalkyl, (C₁-C₄)haloalkyl, cyano, —CO(C₁-C₄)alkyl, —CO₂H, —CO₂R⁷, —CONH₂, —CONHR⁷, —CONR⁷R⁸, HO₂C(C₁-C₂)alkyl-, R⁷O₂C(C₁-C₂)alkyl-, —SR⁷, —SO₂(C₁-C₄)alkyl, —SO₂NH₂, —SO₂NHR⁷, —SO₂NR⁷R⁸, nitro, amino, —NHR⁷, —NR⁷R⁸, amino(C₁-C₂)alkyl-, R⁷HN(C₁-C₂)alkyl-, R⁷R⁸N(C₁-C₂)alkyl-, —NHCO(C₁-C₄)alkyl, —NHSO₂(C₁-C₄)alkyl, oxo, hydroxyl, —OR⁷, hydroxy(C₁-C₂)alkyl-, or R⁷O(C₁-C₂)alkyl-.

9. The compound or salt according to claim 1, wherein R⁷ is (C₁-C₄)alkyl, phenyl, pyrrolidinyl, piperidinyl, morpholinyl, thiomorpholinyl, piperazinyl, or pyrrolidinyl(C₁-C₂)alkyl, piperidinyl(C₁-C₂)alkyl, morpholinyl(C₁-C₂)alkyl, thiomorpholinyl(C₁-C₂)alkyl, or piperazinyl(C₁-C₂)alkyl, wherein said (C₁-C₄)alkyl is optionally substituted one to three times, independently, by halogen, hydroxyl, (C₁-C₄)alkoxy, amino, (C₁-C₄)alkylamino, ((C₁-C₄)alkyl)((C₁-C₄)alkyl)amino, —CO₂H, —CO₂(C₁-C₄)alkyl, —CONH₂, —CONH(C₁-C₄)alkyl, or —CON((C₁-C₄)alkyl)((C₁-C₄)alkyl); and wherein any pyrrolidinyl, piperidinyl, morpholinyl, thiomorpholinyl, or piperazinyl is optionally substituted by (C₁-C₄)alkyl.

10. The compound or salt according to claim 1, wherein R⁷ and R⁸ taken together with the nitrogen to which they are attached represent pyrrolidinyl, piperidinyl, morpholinyl, thiomorpholinyl, piperazinyl, or hexahydro-1H-1,4-diazepinyl, each optionally substituted one or two times, independently, by halogen, (C₁-C₄)alkyl, (C₁-C₄)haloalkyl, amino, (C₁-C₄)alkylamino, (C₁-C₄)alkyl)((C₁-C₄)alkyl)amino, hydroxyl, oxo, (C₁-C₄)alkoxy, or (C₁-C₄)alkoxy(C₁-C₄)alkyl.

11. A compound which is:
N-methyl-3-({6-[(3-methylphenyl)amino]-4-pyrimidinyl}amino)benzenesulfonamide;
3-({6-[(3-chlorophenyl)amino]-4-pyrimidinyl}amino)-N-methylbenzenesulfonamide;
N-methyl-3-{[6-(methylamino)-4-pyrimidinyl]amino}benzenesulfonamide;
3-{[6-(ethylamino)-4-pyrimidinyl]amino}-N-methylbenzenesulfonamide;
3,3′-(4,6-pyrimidinediyldiimino)bis(N-methylbenzenesulfonamide);
3-({6-[(4-chlorophenyl)amino]-4-pyrimidinyl}amino)-5-(dimethylamino)-N-methylbenzenesulfonamide;
3-chloro-5-({6-[(4-chlorophenyl)amino]-4-pyrimidinyl}amino)-N-methylbenzenesulfonamide;
3-({6-[(4-chlorophenyl)amino]-4-pyrimidinyl}amino)-N-methyl-4-(propyloxy)benzenesulfonamide;
3-({6-[(4-chlorophenyl)amino]-4-pyrimidinyl}amino)-4-(ethyloxy)-N-methylbenzenesulfonamide;
3-({6-[(4-chlorophenyl)amino]-4-pyrimidinyl}amino)-N-methyl-4-[(2-methylpropyl)oxy]benzenesulfonamide;
3-({6-[(4-chlorophenyl)amino]-4-pyrimidinyl}amino)-4-[(1,2-dimethylpropyl)oxy]-N-methylbenzenesulfonamide;
4-chloro-3-({6-[(4-chlorophenyl)amino]-4-pyrimidinyl}amino)-N-methylbenzenesulfonamide;
3-({6-[(4-chlorophenyl)amino]-4-pyrimidinyl}amino)-N-methyl-4-[(2,2,2-trifluoroethyl)oxy]-benzenesulfonamide;
3-({6-[(4-chlorophenyl)amino]-4-pyrimidinyl}amino)-4-(cyclohexyloxy)-N-methylbenzenesulfonamide;
3-({6-[(4-chlorophenyl)amino]-4-pyrimidinyl}amino)-4-[(1-ethylpropyl)oxy]-N-methylbenzenesulfonamide;
3-({6-[(4-chlorophenyl)amino]-4-pyrimidinyl}amino)-N-methyl-4-[(3,3,3-trifluoropropyl)oxy]-benzenesulfonamide;
3-({6-[(4-chlorophenyl)amino]-4-pyrimidinyl}amino)-4-(cyclopentyloxy)-N-methylbenzenesulfonamide;
5-(6-(4-chlorophenylamino)pyrimidin-4-ylamino)-2-fluoro-4-methoxy-N-methylbenzenesulfonamide;
3-({6-[(4-chlorophenyl)amino]-4-pyrimidinyl}amino)-N-methyl-4-[methyl(2,2,2-trifluoroethyl)amino]benzenesulfonamide;
1-[6-(4-chloro-phenylamino)-pyrimidin-4-yl]-3,3-dimethyl-2,3-dihydro-1H-indole-6-sulfonic acid methylamide;
3-({6-[(4-chlorophenyl)amino]-4-pyrimidinyl}amino)-N-methyl-4-[(2,2,2-trifluoro-1-methylethyl)oxy]benzenesulfonamide;
5-(6-(4-chlorophenylamino)pyrimidin-4-ylamino)-2-fluoro-N-methyl-4-(2,2,2-trifluoroethoxy)benzenesulfonamide;
4-amino-3-({6-[(4-chlorophenyl)amino]-4-pyrimidinyl}amino)-N-methylbenzenesulfonamide;
5-[6-(4-chloro-phenylamino)-pyrimidin-4-ylamino]-4-dimethylamino-2-fluoro-N-methyl-benzenesulfonamide;
3-({6-[(4-chlorophenyl)amino]-4-pyrimidinyl}amino)-4-(3,3-difluoro-1-piperidinyl)-N-methylbenzenesulfonamide;
3-({6-[(4-chlorophenyl)amino]-4-pyrimidinyl}amino)-N-methyl-4-{[2,2,2-trifluoro-1-(trifluoromethyl)ethyl]oxy}benzenesulfonamide;
4-(dimethylamino)-3-({6-[(3-fluorophenyl)amino]-4-pyrimidinyl}amino)-N-methylbenzenesulfonamide;
3-({6-[(3-fluorophenyl)amino]-4-pyrimidinyl}amino)-N-methyl-4-(4-morpholinyl)benzenesulfonamide;
1-{6-[(3-fluorophenyl)amino]-4-pyrimidinyl}-N-methyl-2,3-dihydro-1H-indole-6-sulfonamide;
3-({6-[(3-fluorophenyl)amino]-4-pyrimidinyl}amino)-N-methyl-4-(methyloxy)benzenesulfonamide;
N-methyl-3-[(6-{[4-(1-methylethyl)phenyl]amino}-4-pyrimidinyl)amino]-4-(methylthio)benzenesulfonamide;
3-[(6-{[3-chloro-4-(methyloxy)phenyl]amino}-4-pyrimidinyl)amino]-N-methyl-4-[(2,2,2-trifluoroethyl)oxy]benzenesulfonamide;
3-[(6-{[3-chloro-4-(methyloxy)phenyl]amino}-4-pyrimidinyl)amino]-N-methyl-4-(methyloxy)benzenesulfonamide;
N-methyl-4-(methyloxy)-3-({6-[(4-{[2-(methyloxy)ethyl]oxy}phenyl)amino]-4-pyrimidinyl}amino)benzenesulfonamide;
N-methyl-3-({6-[(4-{[2-(methyloxy)ethyl]oxy}phenyl)amino]-4-pyrimidinyl}amino)-4-[(2,2,2-trifluoroethyl)oxy]benzenesulfonamide;
N-methyl-4-(methyloxy)-3-[(6-{[4-(2,2,2-trifluoroethyl)phenyl]amino}-4-pyrimidinyl)amino]benzenesulfonamide;
N-methyl-4-[(2,2,2-trifluoroethyl)oxy]-3-[(6-{[4-(2,2,2-trifluoroethyl)phenyl]amino}-4-pyrimidinyl)amino]benzenesulfonamide;
N-methyl-3-[(6-{[4-(2,2,2-trifluoroethyl)phenyl]amino}-4-pyrimidinyl)amino]-4-[(2,2,2-trifluoroethyl)thio]benzenesulfonamide;
4-[(6-{[5-[(methylamino)sulfonyl]-2-(methylthio)phenyl]amino}-4-pyrimidinyl)amino]-N-[2-(methyloxy)ethyl]benzamide;
N-methyl-4-(methyloxy)-3-[(6-{[4-(1H-pyrazol-1-yl)phenyl]amino}-4-pyrimidinyl)amino]benzenesulfonamide;
N-methyl-3-[(6-{[4-(1H-pyrazol-1-yl)phenyl]amino}-4-pyrimidinyl)amino]-4-[(2,2,2-trifluoroethyl)oxy]benzenesulfonamide;
N-methyl-4-[(2,2,2-trifluoroethyl)oxy]-3-{[6-({4-[(2,2,2-trifluoroethyl)oxy]phenyl}amino)-4-pyrimidinyl]amino}benzenesulfonamide;
N-methyl-4-[(2,2,2-trifluoroethyl)oxy]-3-[(6-{[4-(trifluoromethyl)phenyl]amino}-4-pyrimidinyl)amino]benzenesulfonamide;
3-({6-[(3,4-difluorophenyl)amino]-4-pyrimidinyl}amino)-4-fluoro-N-methylbenzenesulfonamide;
3-({6-[(3,4-difluorophenyl)amino]-4-pyrimidinyl}amino)-N-methyl-4-[(2,2,2-trifluoro-1-methylethyl)oxy]benzenesulfonamide;
1-{6-[(3,4-difluorophenyl)amino]-4-pyrimidinyl}-N,3,3-trimethyl-2,3-dihydro-1H-indole-6-sulfonamide;
3-[6-(6-bromo-4-methyl-pyridin-2-ylamino)-pyrimidin-4-ylamino]-N-methyl-4-(2,2,2-trifluoro-ethoxy)-benzenesulfonamide;
3-({6-[(3,5-dichloro-2-pyridinyl)amino]-4-pyrimidinyl}amino)-N-methyl-4-[(2,2,2-trifluoroethyl)oxy]benzenesulfonamide;
3-{[6-(3-biphenylylamino)-4-pyrimidinyl]amino}-N-methylbenzenesulfonamide;
N-methyl-3-({6-[(4-methylphenyl)amino]-4-pyrimidinyl}amino)benzenesulfonamide;
3-{[6-({3-[(methylamino)sulfonyl]phenyl}amino)-4-pyrimidinyl]amino}benzamide;
3-({6-[(3-acetylphenyl)amino]-4-pyrimidinyl}amino)-N-methylbenzenesulfonamide;
N-methyl-3-[(6-{[3-(methyloxy)phenyl]amino}-4-pyrimidinyl)amino]benzenesulfonamide;
N-(3-{[6-({3-[(methylamino)sulfonyl]phenyl}amino}-4-pyrimidinyl]amino phenyl)acetamide;
N-methyl-3-{[6-(phenylamino)-4-pyrimidinyl]amino}benzenesulfonamide;
4-{[6-({3-[(methylamino)sulfonyl]phenyl}amino)-4-pyrimidinyl]amino}benzamide; 3-({6-[(4-chlorophenyl)amino]-4-pyrimidinyl}amino)-N-methylbenzenesulfonamide;
N-methyl-3-[(6-{[3-(trifluoromethyl)phenyl]amino}-4-pyrimidinyl)amino]benzenesulfonamide;
N-methyl-3-({6-[(2-methyl-1,2,3,4-tetrahydro-7-isoquinolinyl)amino]-4-pyrimidinyl}amino)benzenesulfonamide;
3-({6-[(2-fluorophenyl)amino]-4-pyrimidinyl}amino)-N-methylbenzenesulfonamide;
N-methyl-3-[(6-{[3-(4-morpholinylsulfonyl)phenyl]amino}-4-pyrimidinyl)amino]benzenesulfonamide;
3-{[6-({3-[(ethylamino)sulfonyl]phenyl}amino)-4-pyrimidinyl]amino}-N-methylbenzenesulfonamide;
N-methyl-3-[(6-{[3-(methylsulfonyl)phenyl]amino}-4-pyrimidinyl)amino]benzenesulfonamide;
3-[6-(1H-indazol-6-ylamino)-pyrimidin-4-ylamino]-N-methyl-benzenesulfonamide;
3-{[6-({3-[(methylamino)sulfonyl]phenyl}amino)-4-pyrimidinyl]amino}-N-phenylbenzamide;
3-{[6-({3-[(dimethylamino)sulfonyl]phenyl}amino)-4-pyrimidinyl]amino}-N-methylbenzenesulfonamide;
3-[(6-{[3-(aminosulfonyl)phenyl]amino}-4-pyrimidinyl)amino]-N-methylbenzenesulfonamide;
3-{[6-({3-[(methylamino)sulfonyl]phenyl}amino)-4-pyrimidinyl]amino}-N-(1-methylethyl)benzenesulfonamide;
3-({6-[(4-acetylphenyl)amino]-4-pyrimidinyl}amino)-N-methylbenzene sulfonamide;
N-methyl-3-[(6-{[4-(methylsulfonyl)phenyl]amino}-4-pyrimidinyl)amino]benzenesulfonamide;
N-(4-{[6-({3-[(methylamino)sulfonyl]phenyl}amino)-4-pyrimidinyl]amino}phenyl)acetamide;
N-(3-{[6-({3-[(methylamino)sulfonyl]phenyl}amino)-4-pyrimidinyl]amino}phenyl)propanamide;
4-{[6-({3-[(methylamino)sulfonyl]phenyl}amino)-4-pyrimidinyl]amino}-N-phenylbenzamide;
3-({6-[(1,1-dioxido-2,3-dihydro-1,2-benzisothiazol-6-yl)amino]-4-pyrimidinyl}amino)-N-methylbenzene sulfonamide;
N-methyl-3-({6-[(2-oxo-2,3-dihydro-1H-indol-6-yl)amino]-4-pyrimidinyl}amino)benzenesulfonamide;
N-methyl-3-[6-(2-methyl-benzothiazol-5-ylamino)-pyrimidin-4-ylamino]-benzenesulfonamide;
N-methyl-3-({6-[(3-nitrophenyl)amino]-4-pyrimidinyl}amino)benzenesulfonamide;
N-methyl-3-[(6-{[4-(4-morpholinylcarbonyl)phenyl]amino}-4-pyrimidinyl)amino]benzenesulfonamide;
N-methyl-4-{[6-({3-[(methylamino)sulfonyl]phenyl}amino)-4-pyrimidinyl]amino}benzamide;
3-[6-(2,3-dihydro-benzo[1,4]dioxin-6-ylamino)-pyrimidin-4-ylamino]-N-methyl-benzenesulfonamide;
N-methyl-3-[(6-{[4-(methyloxy)phenyl]amino}-4-pyrimidinyl)amino]benzenesulfonamide;
N-methyl-3-[(6-{[4-(4-morpholinyl)phenyl]amino}-4-pyrimidinyl)amino]benzenesulfonamide;
3-[(6-{[4-(1,1-dimethylethyl)phenyl]amino}-4-pyrimidinyl)amino]-N-methylbenzenesulfonamide;
N-methyl-3-[(6-{[3-(4-morpholinyl)phenyl]amino}-4-pyrimidinyl)amino]benzenesulfonamide;
3-({6-[(3-bromo-5-methylphenyl)amino]-4-pyrimidinyl}amino)-N-methylbenzenesulfonamide;
3-[(6-{[4-(dimethylamino)phenyl]amino}-4-pyrimidinyl)amino]-N-methylbenzenesulfonamide;
3-[(6-{[3-(dimethylamino)phenyl]amino}-4-pyrimidinyl)amino]-N-methylbenzenesulfonamide;
methyl 4-{[6-({3-[(methylamino)sulfonyl]phenyl}amino)-4-pyrimidinyl]amino}benzoate;
1-methylethyl 4-{[6-({3-[(methylamino)sulfonyl]phenyl}amino)-4-pyrimidinyl]amino}benzoate;
3-({6-[(4-chloro-3-methylphenyl)amino]-4-pyrimidinyl}amino)-N-methylbenzenesulfonamide;
3-({6-[(4-fluoro-3-methylphenyl)amino]-4-pyrimidinyl}amino)-N-methylbenzenesulfonamide;
3-{[6-(1H-indol-6-ylamino)-4-pyrimidinyl]amino}-N-methylbenzenesulfonamide;
N-methyl-3-{[6-({3-[(methylsulfonyl)amino]phenyl}amino)-4-pyrimidinyl]amino}benzenesulfonamide;
N-methyl-3-({6-[(3-methyl-1H-indazol-6-yl)amino]-4-pyrimidinyl}amino)benzenesulfonamide;
3-({6-[(4-{[2-(diethylamino)ethyl]oxy}phenyl)amino]-4-pyrimidinyl}amino)-N-methylbenzenesulfonamide;
1-methylethyl[(3-{[6-({3-[(methylamino)sulfonyl]phenyl}amino)-4-pyrimidinyl]amino}phenyl)oxy]acetate;
3-[6-(benzothiazol-6-ylamino)-pyrimidin-4-ylamino]-N-methyl-benzenesulfonamide;
3-[6-(1H-indol-5-ylamino)-pyrimidin-4-ylamino]-N-methyl-benzenesulfonamide;
3-{[6-(1,3-benzothiazol-5-ylamino)-4-pyrimidinyl]amino}-N-methylbenzenesulfonamide;
3-({6-[(3-fluoro-4-methylphenyl)amino]-4-pyrimidinyl}amino)-N-methylbenzenesulfonamide;
3-({6-[(3-fluorophenyl)amino]-4-pyrimidinyl}amino)-N-methylbenzenesulfonamide;
3-[(6-{[3-fluoro-4-(trifluoromethyl)phenyl]amino}-4-pyrimidinyl)amino]-N-methylbenzenesulfonamide;
N-methyl-3-[(6-{[4-(methyloxy)-3-(trifluoromethyl)phenyl]amino}-4-pyrimidinyl)amino]benzenesulfonamide;
3-({6-[(4-chloro-3-fluorophenyl)amino]-4-pyrimidinyl}amino)-N-methylbenzenesulfonamide;
3-[(6-{[3-fluoro-4-(methyloxy)phenyl]amino}-4-pyrimidinyl)amino]-N-methylbenzenesulfonamide;
N-methyl-3-[(6-{[4-methyl-3-(trifluoromethyl)phenyl]amino}-4-pyrimidinyl)amino]benzenesulfonamide;
3-[(6-{[4-chloro-3-(trifluoromethyl)phenyl]amino}-4-pyrimidinyl)amino]-N-methylbenzenesulfonamide;
N-methyl-3-[(6-{[4-(2,2,2-trifluoroethyl)phenyl]amino}-4-pyrimidinyl)amino]benzenesulfonamide;
N-methyl-4-(methylthio)-3-({6-[(2-oxo-1,2,3,4-tetrahydro-7-quinolinyl)amino]-4-pyrimidinyl}amino)benzenesulfonamide;
4-[(6-{[5-[(methylamino)sulfonyl]-2-(methylthio)phenyl]amino}-4-pyrimidinyl)amino]benzoic acid;
3-({6-[(4-chlorophenyl)amino]-4-pyrimidinyl}amino)-4-(diethylamino)-N-methylbenzenesulfonamide;
3-({6-[(4-chlorophenyl)amino]-4-pyrimidinyl}amino)-4-(2,5-dimethyl-1-pyrrolidinyl)-N-methylbenzenesulfonamide;
3-({6-[(4-chlorophenyl)amino]-4-pyrimidinyl}amino)-N-methyl-4-(2-methyl-1-pyrrolidinyl)benzenesulfonamide;
3-({6-[(4-chlorophenyl)amino]-4-pyrimidinyl}amino)-N,4-dimethylbenzenesulfonamide;
3-(6-(4-chlorophenylamino)pyrimidin-4-ylamino)-4-(isobutylthio)-N-methylbenzenesulfonamide;
4-(isobutylthio)-N-methyl-3-(6-(4-(trifluoromethyl)phenylamino)pyrimidin-4-ylamino)benzenesulfonamide;
4-(isobutylthio)-3-(6-(4-isopropylphenylamino)pyrimidin-4-ylamino)-N-methylbenzenesulfonamide;
3-{[6-({4-[(difluoromethyl)oxy]phenyl}amino)-4-pyrimidinyl]amino}-N-methyl-4-[(2,2,2-trifluoroethyl)oxy]benzenesulfonamide;
N-methyl-4-[(2,2,2-trifluoroethyl)oxy]-3-{[6-({4-[(trifluoromethyl)oxy]phenyl}amino)-4-pyrimidinyl]amino}benzenesulfonamide;
3-({6-[(3,4-difluorophenyl)amino]-4-pyrimidinyl}amino)-N-methyl-4-[(2,2,2-trifluoroethyl)oxy]benzenesulfonamide;
3-({6-[(4-cyanophenyl)amino]-4-pyrimidinyl}amino)-N-methyl-4-[(2,2,2-trifluoroethyl)oxy]benzenesulfonamide;
3-(6-(4-chlorophenylamino)pyrimidin-4-ylamino)-4-(ethylthio)-N-methylbenzenesulfonamide;
4-(ethylthio)-N-methyl-3-(6-(4-(trifluoromethyl)phenylamino)pyrimidin-4-ylamino)benzenesulfonamide;
4-(ethylthio)-3-(6-(4-isopropylphenylamino)pyrimidin-4-ylamino)-N-methylbenzenesulfonamide;
3-(6-(4-chlorophenylamino)pyrimidin-4-ylamino)-N-methyl-4-(2,2,2-trifluoroethylthio)benzenesulfonamide;
N-methyl-4-(2,2,2-trifluoroethylthio)-3-(6-(4-(trifluoromethyl)phenylamino)pyrimidin-4-ylamino)benzenesulfonamide;
3-(6-(4-isopropylphenylamino)pyrimidin-4-ylamino)-N-methyl-4-(2,2,2-trifluoroethylthio)benzenesulfonamide;
4-fluoro-N-methyl-3-{[6-({4-[(trifluoromethyl)oxy]phenyl}amino)-4-pyrimidinyl]amino}benzenesulfonamide;
3-{[6-({4-[(difluoromethyl)oxy]phenyl}amino)-4-pyrimidinyl]amino}-4-fluoro-N-methylbenzenesulfonamide;
4-chloro-N-methyl-3-[(6-{[4-(trifluoromethyl)phenyl]amino}-4-pyrimidinyl)amino]benzenesulfonamide;
3-({6-[(4-cyanophenyl)amino]-4-pyrimidinyl}amino)-N-methyl-4-(methylsulfonyl)benzenesulfonamide;
3-({6-[(3,4-difluorophenyl)amino]-4-pyrimidinyl}amino)-N-methyl-4-(methylsulfonyl)benzenesulfonamide;
3-(6-(1H-indazol-5-ylamino)pyrimidin-4-ylamino)-N-methyl-4-(methylsulfonyl)benzenesulfonamide;
3-(6-(4-(cyanomethyl)phenylamino)pyrimidin-4-ylamino)-N-methyl-4-(methylsulfonyl)benzenesulfonamide;
4-(tert-butylsulfonyl)-3-(6-(4-chlorophenylamino)pyrimidin-4-ylamino)-N-methylbenzenesulfonamide;
3-({6-[(4-chlorophenyl)amino]-4-pyrimidinyl}amino)-N-methyl-4-[(2,2,2-trifluoro-1,1-dimethylethyl)oxy]benzenesulfonamide;
3-({6-[(3-bromophenyl)amino]-4-pyrimidinyl}amino)-N-methylbenzenesulfonamide;
3-({6-[(3-bromo-4-chlorophenyl)amino]-4-pyrimidinyl}amino)-N-methylbenzenesulfonamide;
3-[6-(3,4-dimethoxy-phenylamino)-pyrimidin-4-ylamino]-N-methyl-4-methylsulfanyl-benzenesulfonamide;
N-methyl-4-methylsulfanyl-3-[6-(3,4,5-trimethoxy-phenylamino)-pyrimidin-4-ylamino]-benzenesulfonamide;
3-[6-(3,5-dimethoxy-phenylamino)-pyrimidin-4-ylamino]-N-methyl-4-methylsulfanyl-benzenesulfonamide;
3-[6-(4-cyano-phenylamino)-pyrimidin-4-ylamino]-N-methyl-4-methylsulfanyl-benzenesulfonamide;
3-[6-(benzo[1,3]dioxol-5-ylamino)-pyrimidin-4-ylamino]-N-methyl-4-methylsulfanyl-benzenesulfonamide;
3-[6-(benzothiazol-6-ylamino)-pyrimidin-4-ylamino]-N-methyl-4-methylsulfanyl-benzenesulfonamide;
N-methyl-3-[6-(2-methyl-benzothiazol-5-ylamino)-pyrimidin-4-ylamino]-4-methylsulfanyl-benzenesulfonamide;
3-[6-(3-chloro-4-hydroxy-phenylamino)-pyrimidin-4-ylamino]-N-methyl-4-methylsulfanyl-benzenesulfonamide;
3-[6-(3,4-difluoro-phenylamino)-pyrimidin-4-ylamino]-N-methyl-4-methylsulfanyl-benzenesulfonamide;
N-methyl-4-methylsulfanyl-3-[6-(4-morpholin-4-yl-phenylamino)-pyrimidin-4-ylamino]-benzenesulfonamide;
3-[6-(2,3-dihydro-benzo[1,4]dioxin-6-ylamino)-pyrimidin-4-ylamino]-N-methyl-4-methylsulfanyl-benzenesulfonamide;
N-methyl-4-methylsulfanyl-3-[6-(4-piperidin-1-yl-phenylamino)-pyrimidin-4-ylamino]-benzenesulfonamide;
3-[6-(3-ethynyl-phenylamino)-pyrimidin-4-ylamino]-N-methyl-4-methylsulfanyl-benzenesulfonamide;
3-[6-(3,5-dichloro-4-hydroxy-phenylamino)-pyrimidin-4-ylamino]-N-methyl-4-methylsulfanyl-benzenesulfonamide;
N-methyl-4-methylsulfanyl-3-{6-[3-(2-methyl-thiazol-4-O-phenylamino]-pyrimidin-4-ylamino}-benzenesulfonamide;
3-(6-(3-methoxy-5-(trifluoromethyl)phenylamino)pyrimidin-4-ylamino)-N-methyl-4-(methylthio)benzenesulfonamide;
3-[6-(1H-indol-5-ylamino)-pyrimidin-4-ylamino]-N-methyl-4-methylsulfanyl-benzenesulfonamide;
N-methyl-4-methylsulfanyl-3-[6-(quinolin-6-ylamino)-pyrimidin-4-ylamino]-benzenesulfonamide;
3-[6-(3-chloro-4-cyano-phenylamino)-pyrimidin-4-ylamino]-N-methyl-4-methylsulfanyl-benzenesulfonamide;
N-methyl-4-methylsulfanyl-3-[6-(4-[1,2,4]triazol-4-ylmethyl-phenylamino)-pyrimidin-4-ylamino]-benzenesulfonamide;
3-[6-(1H-indazol-5-ylamino)-pyrimidin-4-ylamino]-N-methyl-4-methylsulfanyl-benzenesulfonamide;
3-[6-(1H-indol-6-ylamino)-pyrimidin-4-ylamino]-N-methyl-4-methylsulfanyl-benzenesulfonamide;
N-methyl-4-(methylthio)-3-(6-(4-(piperazin-1-yl)phenylamino)pyrimidin-4-ylamino)benzenesulfonamide;
N-methyl-3-(6-(4-methyl-2-oxo-1,2-dihydroquinolin-7-ylamino)pyrimidin-4-ylamino)-4-(methylthio)benzenesulfonamide;
3-(6-(1-acetylindolin-6-ylamino)pyrimidin-4-ylamino)-N-methyl-4-(methylthio)benzenesulfonamide;
N-methyl-3-[6-(2-methyl-4-oxo-4H-chromen-7-ylamino)-pyrimidin-4-ylamino]-4-methylsulfanyl-benzenesulfonamide;
3-[6-(4-cyanomethyl-phenylamino)-pyrimidin-4-ylamino]-N-methyl-4-methylsulfanyl-benzenesulfonamide;
N-methyl-4-methylsulfanyl-3-[6-(5-oxo-5,6,7,8-tetrahydro-naphthalen-2-ylamino)-pyrimidin-4-ylamino]-benzenesulfonamide;
N-methyl-4-methylsulfanyl-3-[6-(3,4,5-trifluoro-phenylamino)-pyrimidin-4-ylamino]-benzenesulfonamide;
N-methyl-3-[6-(4-methyl-2-oxo-2H-chromen-7-ylamino)-pyrimidin-4-ylamino]-4-methylsulfanyl-benzenesulfonamide;
3-[6-(indan-5-ylamino)-pyrimidin-4-ylamino]-N-methyl-4-methylsulfanyl-benzenesulfonamide;
3-[6-(1H-indazol-6-ylamino)-pyrimidin-4-ylamino]-N-methyl-4-methylsulfanyl-benzenesulfonamide;
N-methyl-3-(6-(2-methyl-1,3-dioxoisoindolin-5-ylamino)pyrimidin-4-ylamino)-4-(methylthio)benzenesulfonamide;
3-[6-(3,5-dimethoxy-phenylamino)-pyrimidin-4-ylamino]-N-methyl-benzenesulfonamide;
N-methyl-3-[6-(3,4,5-trimethoxy-phenylamino)-pyrimidin-4-ylamino]-benzenesulfonamide;
3-[6-(3-ethynyl-phenylamino)-pyrimidin-4-ylamino]-N-methyl-benzenesulfonamide;
3-[6-(benzo[1,3]dioxol-5-ylamino)-pyrimidin-4-ylamino]-N-methyl-benzenesulfonamide;
3-[6-(3-chloro-4-hydroxy-phenylamino)-pyrimidin-4-ylamino]-N-methyl-benzenesulfonamide;
3-[6-(3,4-difluoro-phenylamino)-pyrimidin-4-ylamino]-N-methyl-benzenesulfonamide;
N-methyl-3-[6-(4-piperidin-1-yl-phenylamino)-pyrimidin-4-ylamino]-benzenesulfonamide;
3-[6-(4-cyano-phenylamino)-pyrimidin-4-ylamino]-N-methyl-benzenesulfonamide;
N-methyl-3-[6-(2-methyl-4-oxo-4H-chromen-7-ylamino)-pyrimidin-4-ylamino]-benzenesulfonamide;
3-[6-(3,5-dichloro-4-hydroxy-phenylamino)-pyrimidin-4-ylamino]-N-methyl-benzenesulfonamide;
N-methyl-3-{6-[3-(2-methyl-thiazol-4-yl)-phenylamino]-pyrimidin-4-ylamino}-benzenesulfonamide;
3-[6-(1H-indazol-5-ylamino)-pyrimidin-4-ylamino]-N-methyl-benzenesulfonamide;
N-methyl-3-[6-(5-oxo-5,6,7,8-tetrahydro-naphthalen-2-ylamino)-pyrimidin-4-ylamino]-benzenesulfonamide;
3-[6-(4-cyanomethyl-phenylamino)-pyrimidin-4-ylamino]-N-methyl-benzenesulfonamide;
N-methyl-3-[6-(4-methyl-2-oxo-2H-chromen-7-ylamino)-pyrimidin-4-ylamino]-benzenesulfonamide;
3-[6-(1-acetyl-2,3-dihydro-1H-indol-6-ylamino)-pyrimidin-4-ylamino]-N-methyl-benzenesulfonamide;
3-[6-(3-methoxy-5-trifluoromethyl-phenylamino)-pyrimidin-4-ylamino]-N-methyl-benzenesulfonamide;
N-methyl-3-[6-(4-methyl-2-oxo-1,2-dihydro-quinolin-7-ylamino)-pyrimidin-4-ylamino]-benzenesulfonamide;
N-methyl-3-[6-(3,4,5-trifluoro-phenylamino)-pyrimidin-4-ylamino]-benzenesulfonamide;
3-[6-(indan-5-ylamino)-pyrimidin-4-ylamino]-N-methyl-benzenesulfonamide
3-[6-(4-chloro-phenylamino)-pyrimidin-4-ylamino]-N-methyl-4-(propane-2-sulfonyl)-benzenesulfonamide;
3-(6-(3-bromo-5-methylphenylamino)pyrimidin-4-ylamino)-N-methyl-4-(methylsulfonyl)benzenesulfonamide;
3-(6-(1H-indol-6-ylamino)pyrimidin-4-ylamino)-N-methyl-4-(methylsulfonyl)benzenesulfonamide;
3-(6-(3-ethynylphenylamino)pyrimidin-4-ylamino)-N-methyl-4-(methylsulfonyl)benzenesulfonamide;
3-[6-(indan-5-ylamino)-pyrimidin-4-ylamino]-4-methanesulfonyl-N-methyl-benzenesulfonamide;
3-[6-(benzothiazol-6-ylamino)-pyrimidin-4-ylamino]-4-methanesulfonyl-N-methyl-benzenesulfonamide;
4-methanesulfonyl-N-methyl-3-[6-(5-oxo-5,6,7,8-tetrahydro-naphthalen-2-ylamino)-pyrimidin-4-ylamino]-benzenesulfonamide;
N-methyl-3-(6-(2-methylbenzo[d]thiazol-5-ylamino)pyrimidin-4-ylamino)-4-(methylsulfonyl)benzenesulfonamide;
N-methyl-4-(methylsulfonyl)-3-[(6-{[4-(1H-1,2,4-triazol-1-ylmethyl)phenyl]amino}-4-pyrimidinyl)amino]benzenesulfonamide;
3-[6-(1H-indol-5-ylamino)-pyrimidin-4-ylamino]-4-methanesulfonyl-N-methyl-benzenesulfonamide;
4-methanesulfonyl-N-methyl-3-[6-(2-methyl-4-oxo-4H-chromen-7-ylamino)-pyrimidin-4-ylamino]-benzenesulfonamide;
5-({6-[(4-chlorophenyl)amino]-4-pyrimidinyl}amino)-2-fluoro-N-methylbenzenesulfonamide;
5-(6-(4-chlorophenylamino)pyrimidin-4-ylamino)-2-fluoro-N-methyl-4-(1,1,1-trifluoropropan-2-yloxy)benzenesulfonamide;
1-{6-[(4-chlorophenyl)amino]-4-pyrimidinyl}-N-methyl-2,3-dihydro-1H-indole-6-sulfonamide;
3-[(6-{[3,4-bis(methyloxy)phenyl]amino}-4-pyrimidinyl)amino]-N-methylbenzenesulfonamide;
3-({6-[(3,4-dichlorophenyl)amino]-4-pyrimidinyl}amino)-N-methylbenzenesulfonamide;
3-({6-[(3,4-dimethylphenyl)amino]-4-pyrimidinyl}amino)-N-methylbenzenesulfonamide;
N-methyl-3-[(6-{[3-(1-methylethyl)phenyl]amino}-4-pyrimidinyl)amino]benzenesulfonamide;
3-[(6-{[3-(1,1-dimethylethyl)phenyl]amino}-4-pyrimidinyl)amino]-N-methylbenzenesulfonamide;
3-[(6-{[3-(ethyloxy)phenyl]amino}-4-pyrimidinyl)amino]-N-methylbenzenesulfonamide;
3-({6-[(4-fluorophenyl)amino]-4-pyrimidinyl}amino)-N-methylbenzenesulfonamide;
N-methyl-3-[(6-{[3-(1-pyrrolidinyl)phenyl]amino}-4-pyrimidinyl)amino]benzenesulfonamide;
N-methyl-3-[(6-{[3-(4-methyl-1-piperazinyl)phenyl]amino}-4-pyrimidinyl)amino]benzenesulfonamide;
3-({6-[(3,5-dichlorophenyl)amino]-4-pyrimidinyl}amino)-N-methylbenzenesulfonamide;
N-methyl-3-({6-[(2-oxo-2,3-dihydro-1H-indol-5-yl)amino]-4-pyrimidinyl}amino)benzenesulfonamide;
N-methyl-3-({6-[(2-oxo-2,3-dihydro-1,3-benzoxazol-6-yl)amino]-4-pyrimidinyl}amino)benzenesulfonamide;
N-methyl-3-({6-[(2-oxo-2,3-dihydro-1H-benzimidazol-5-yl)amino]-4-pyrimidinyl}amino)benzenesulfonamide;
N-methyl-3-({6-[(2-oxo-1,2,3,4-tetrahydro-7-quinolinyl)amino]-4-pyrimidinyl}amino)benzenesulfonamide;
3-({6-[(3-bromo-5-chlorophenyl)amino]-4-pyrimidinyl}amino)-N-methylbenzenesulfonamide;
3-({6-[(3,5-dimethylphenyl)amino]-4-pyrimidinyl}amino)-N-methylbenzenesulfonamide;
N-methyl-3-{[6-({4-[(methylamino)sulfonyl]phenyl}amino)-4-pyrimidinyl]amino}benzenesulfonamide;
N-methyl-3-[(6-{[3-(1-pyrrolidinylmethyl)phenyl]amino}-4-pyrimidinyl)amino]benzenesulfonamide;
N-methyl-3-({6-[(4-{[2-(4-morpholinyl)ethyl]oxy}phenyl)amino]-4-pyrimidinyl}amino)benzenesulfonamide;
3-({6-[(4-{[2-(dimethylamino)ethyl]oxy}phenyl)amino]-4-pyrimidinyl}amino)-N-methylbenzenesulfonamide;
N-methyl-3-{[6-({3-[(4-methyl-1-piperazinyl)methyl]phenyl}amino)-4-pyrimidinyl]amino}benzenesulfonamide;
N-methyl-3-[(6-{[4-(trifluoromethyl)phenyl]amino}-4-pyrimidinyl)amino]benzenesulfonamide;
N-methyl-3-[(6-{[4-(1-methylethyl)phenyl]amino}-4-pyrimidinyl)amino]benzenesulfonamide;
N-methyl-3-{[6-({4-[(1-methylethyl)oxy]phenyl}amino)-4-pyrimidinyl]amino}benzenesulfonamide;
3-{[6-({4-[(difluoromethyl)oxy]phenyl}amino)-4-pyrimidinyl]amino}-N-methylbenzenesulfonamide;
N-methyl-3-[(6-{[4-(2-oxo-1-pyrrolidinyl)phenyl]amino}-4-pyrimidinyl)amino]benzenesulfonamide;
3-[(6-{[3-chloro-4-(methyloxy)phenyl]amino}-4-pyrimidinyl)amino]-N-methylbenzenesulfonamide;
3-({6-[(4-cyclopropylphenyl)amino]-4-pyrimidinyl}amino)-N-methylbenzenesulfonamide;
N-methyl-3-[(6-{[4-(1H-pyrazol-1-yl)phenyl]amino}-4-pyrimidinyl)amino]benzenesulfonamide;
3-[(6-{[4-(3,5-dimethyl-1H-pyrazol-1-yl)phenyl]amino}-4-pyrimidinyl)amino]-N-methylbenzenesulfonamide;
3-[(6-{[4-chloro-3-(methyloxy)phenyl]amino}-4-pyrimidinyl)amino]-N-methylbenzenesulfonamide;
N-methyl-3-[(6-{[4-(2-thienyl)phenyl]amino}-4-pyrimidinyl)amino]benzenesulfonamide;
N-methyl-3-[(6-{[4-(2-methyl-1H-imidazol-1-yl)phenyl]amino}-4-pyrimidinyl)amino]benzenesulfonamide;
N-methyl-3-[(6-{[4-(1-methylpropyl)phenyl]amino}-4-pyrimidinyl)amino]benzenesulfonamide;
N-methyl-3-{[6-(6-quinolinylamino)-4-pyrimidinyl]amino}benzenesulfonamide;
N-methyl-3-{[6-({4-[(trifluoromethyl)thio]phenyl}amino)-4-pyrimidinyl]amino}benzenesulfonamide;
3-({6-[(4-bromophenyl)amino]-4-pyrimidinyl}amino)-N-methylbenzenesulfonamide;
N-methyl-3-[(6-{[4-(methylthio)phenyl]amino}-4-pyrimidinyl)amino]benzenesulfonamide;
N-methyl-3-{[6-({4-[(trifluoromethyl)oxy]phenyl}amino)-4-pyrimidinyl]amino}benzenesulfonamide;
3-({6-[(4-chlorophenyl)amino]-4-pyrimidinyl}amino)-4-(dimethylamino)-N-methylbenzenesulfonamide;
4-(dimethylamino)-N-methyl-3-({6-[(3-methylphenyl)amino]-4-pyrimidinyl}amino)benzenesulfonamide;
N-methyl-1-(6-{[4-(trifluoromethyl)phenyl]amino}-4-pyrimidinyl)-2,3-dihydro-1H-indole-6-sulfonamide;
1-{6-[(4-chlorophenyl)amino]-4-pyrimidinyl}-N-methyl-1H-benzimidazole-6-sulfonamide;
3-({6-[(5-bromo-6-methyl-2-pyridinyl)amino]-4-pyrimidinyl}amino)-N-methyl-4-[(2,2,2-trifluoroethyl)oxy]benzenesulfonamide;
3-({6-[(4-chlorophenyl)amino]-4-pyrimidinyl}amino)-N-methyl-4-[(1-methylethyl)oxy]benzenesulfonamide;
3-({6-[(4-chlorophenyl)amino]-4-pyrimidinyl}amino)-N-methyl-4-(4-morpholinyl)benzenesulfonamide;
3-({6-[(4-chlorophenyl)amino]-4-pyrimidinyl}amino)-N-methyl-4-(methyloxy)benzenesulfonamide;
3-({6-[(4-chlorophenyl)amino]-4-pyrimidinyl}amino)-4-[ethyl(methyl)amino]-N-methylbenzenesulfonamide;
3-({6-[(4-chlorophenyl)amino]-4-pyrimidinyl}amino)-4-hydroxy-N-methylbenzenesulfonamide;
3-({6-[(4-chlorophenyl)amino]-4-pyrimidinyl}amino)-4-fluoro-N-methylbenzenesulfonamide;
3-({6-[(4-chlorophenyl)amino]-4-pyrimidinyl}amino)-N-methyl-4-(methylthio)benzenesulfonamide;
3-({6-[(4-chlorophenyl)amino]-4-pyrimidinyl}amino)-N-methyl-4-[(trifluoromethyl)oxy]benzenesulfonamide;
3-({6-[(4-chlorophenyl)amino]-4-pyrimidinyl}amino)-N-methyl-4-[(2R)-2-(trifluoromethyl)-1-pyrrolidinyl]benzenesulfonamide;
3-({6-[(4-chlorophenyl)amino]-4-pyrimidinyl}amino)-4-(3,3-difluoro-1-pyrrolidinyl)-N-methylbenzenesulfonamide;
N-methyl-3-[(6-{[4-(1,3-oxazol-5-yl)phenyl]amino}-4-pyrimidinyl)amino]benzenesulfonamide;
N-methyl-3-({6-[(3-methylphenyl)amino]-4-pyrimidinyl}amino)-4-(4-morpholinyl)benzenesulfonamide;
N-methyl-4-(methyloxy)-3-[(6-{[4-(trifluoromethyl)phenyl]amino}-4-pyrimidinyl)amino]benzenesulfonamide;
N-methyl-4-(methylthio)-3-[(6-{[4-(trifluoromethyl)phenyl]amino}-4-pyrimidinyl)amino]benzenesulfonamide;
3-({6-[(3-bromo-5-methylphenyl)amino]-4-pyrimidinyl}amino)-N-methyl-4-(methyloxy)benzenesulfonamide;
1-{6-[(3-bromo-5-methylphenyl)amino]-4-pyrimidinyl}-N-methyl-2,3-dihydro-1H-indole-6-sulfonamide;
N-methyl-3-{[6-({4-[(2,2,2-trifluoroethyl)oxy]phenyl}amino)-4-pyrimidinyl]amino}-4-[(2,2,2-trifluoroethyl)thio]benzenesulfonamide;
3-({6-[(3,4-difluorophenyl)amino]-4-pyrimidinyl}amino)-N-methyl-4-[(trifluoromethyl)oxy]benzenesulfonamide;
N-methyl-3-{[6-(4-pyridinylamino)-4-pyrimidinyl]amino}benzenesulfonamide;
N-methyl-3-{[6-(3-pyridinylamino)-4-pyrimidinyl]amino}benzenesulfonamide;
N-methyl-3-({6-[(5-methyl-3-pyridinyl)amino]-4-pyrimidinyl}amino)benzenesulfonamide;
N-methyl-3-{[6-(2-pyridinylamino)-4-pyrimidinyl]amino}benzenesulfonamide;
N-methyl-5-{[6-({3-[(methylamino)sulfonyl]phenyl}amino)-4-pyrimidinyl]amino}-3-pyridinesulfonamide;
3-({6-[(5-chloro-2-pyridinyl)amino]-4-pyrimidinyl}amino)-N-methylbenzenesulfonamide;
N-methyl-3-{[6-(1,3-thiazol-2-ylamino)-4-pyrimidinyl]amino}benzenesulfonamide;
N-methyl-3-[(6-{[5-(trifluoromethyl)-2-pyridinyl]amino}-4-pyrimidinyl)amino]benzenesulfonamide;
N-methyl-3-({6-[(5-methyl-1,3-thiazol-2-yl)amino]-4-pyrimidinyl}amino)benzenesulfonamide;
N-methyl-3-{[6-(1,3,4-thiadiazol-2-ylamino)-4-pyrimidinyl]amino}benzenesulfonamide;
3-{[6-(3-isoquinolinylamino)-4-pyrimidinyl]amino}-N-methylbenzenesulfonamide;
N-methyl-3-{[6-(2-quinolinylamino)-4-pyrimidinyl]amino}benzenesulfonamide;
N-methyl-3-{[6-(1,3-oxazol-2-ylamino)-4-pyrimidinyl]amino}benzenesulfonamide;
N-methyl-3-[(6-{[4-(trifluoromethyl)-1,3-thiazol-2-yl]amino}-4-pyrimidinyl)amino]benzenesulfonamide;
methyl (2-{[6-({3-[(methylamino)sulfonyl]phenyl}amino)-4-pyrimidinyl]amino}-1,3-thiazol-4-yl)acetate;
N-methyl-3-[(6-{[4-(1-methylethyl)-1,3-thiazol-2-yl]amino}-4-pyrimidinyl)amino]benzenesulfonamide;
N-methyl-3-({6-[(4-methyl-1,3-oxazol-2-yl)amino]-4-pyrimidinyl}amino)benzenesulfonamide;
N-methyl-4-(methyloxy)-3-{[6-(2-pyridinylamino)-4-pyrimidinyl]amino}benzenesulfonamide;
3-({6-[(5-chloro-2-pyridinyl)amino]-4-pyrimidinyl}amino)-N-methyl-4-(methyloxy)benzenesulfonamide;
3-({6-[(5-chloro-2-pyridinyl)amino]-4-pyrimidinyl}amino)-N-methyl-4-[(2,2,2-trifluoroethyl)oxy]benzenesulfonamide;
N-methyl-3-{[6-(2-pyridinylamino)-4-pyrimidinyl]amino}-4-[(2,2,2-trifluoroethyl)oxy]benzenesulfonamide;
3-({6-[(5-chloro-2-pyridinyl)amino]-4-pyrimidinyl}amino)-N-methyl-4-(methylthio)benzenesulfonamide;
1-{6-[(5-chloro-2-pyridinyl)amino]-4-pyrimidinyl}-N-methyl-2,3-dihydro-1H-indole-6-sulfonamide;
N-methyl-4-[(2,2,2-trifluoroethyl)oxy]-3-[(6-{[5-(trifluoromethyl)-2-pyridinyl]amino}-4-pyrimidinyl)amino]benzenesulfonamide;
N-methyl-3-{[6-(4-pyridinylamino)-4-pyrimidinyl]amino}-4-[(2,2,2-trifluoroethyl)oxy]benzenesulfonamide;
3-({6-[(3-fluoro-2-pyridinyl)amino]-4-pyrimidinyl}amino)-N-methyl-4-[(2,2,2-trifluoroethyl)oxy]benzenesulfonamide;
3-({6-[(5-cyano-2-pyridinyl)amino]-4-pyrimidinyl}amino)-N-methyl-4-[(2,2,2-trifluoroethyl)oxy]benzenesulfonamide;
N-methyl-3-{[6-(4-pyrimidinylamino)-4-pyrimidinyl]amino}-4-[(2,2,2-trifluoroethyl)oxy]benzenesulfonamide;
3-({6-[(5-chloro-3-fluoro-2-pyridinyl)amino]-4-pyrimidinyl}amino)-N-methyl-4-[(2,2,2-trifluoroethyl)oxy]benzenesulfonamide;
N-methyl-4-[(2,2,2-trifluoroethyl)oxy]-3-[(6-{[6-(trifluoromethyl)-3-pyridinyl]amino}-4-pyrimidinyl)amino]benzenesulfonamide;
3-({6-[(5-chloro-4-methyl-2-pyridinyl)amino]-4-pyrimidinyl}amino)-N-methyl-4-[(2,2,2-trifluoroethyl)oxy]benzenesulfonamide;
3-({6-[(4,5-dichloro-2-pyridinyl)amino]-4-pyrimidinyl}amino)-N-methyl-4-[(2,2,2-trifluoroethyl)oxy]benzenesulfonamide;
3-({6-[(5-chloro-6-methyl-2-pyridinyl)amino]-4-pyrimidinyl}amino)-N-methyl-4-[(2,2,2-trifluoroethyl)oxy]benzenesulfonamide;
3-(6-(5-isopropylpyridin-2-ylamino)pyrimidin-4-ylamino)-N-methyl-4-(2,2,2-trifluoroethoxy)benzenesulfonamide;
3-({6-[(5-chloro-2-pyridinyl)amino]-4-pyrimidinyl}amino)-4-fluoro-N-methylbenzenesulfonamide;
4-fluoro-N-methyl-3-[(6-{[5-(trifluoromethyl)-2-pyridinyl]amino}-4-pyrimidinyl)amino]benzenesulfonamide;
4-chloro-3-({6-[(5-chloro-2-pyridinyl)amino]-4-pyrimidinyl}amino)-N-methylbenzenesulfonamide;
3-({6-[(5-chloro-2-pyridinyl)amino]-4-pyrimidinyl}amino)-N-methyl-4-(methylsulfonyl)benzenesulfonamide;
N-methyl-4-(methylsulfonyl)-3-[(6-{[5-(trifluoromethyl)-2-pyridinyl]amino}-4-pyrimidinyl)amino]benzenesulfonamide;
N-methyl-4-(methylsulfonyl)-3-{[6-(6-quinolinylamino)-4-pyrimidinyl]amino}benzenesulfonamide;
3-({6-[(5-chloro-2-pyridinyl)amino]-4-pyrimidinyl}amino)-N-methyl-4-[(2,2,2-trifluoro-1-methylethyl)oxy]benzenesulfonamide;
N-methyl-4-[(2,2,2-trifluoro-1-methylethyl)oxy]-3-[(6-{[5-(trifluoromethyl)-2-pyridinyl]amino}-4-pyrimidinyl)amino]benzenesulfonamide;
4-(tert-butylsulfonyl)-N-methyl-3-(6-(5-(trifluoromethyl)pyridin-2-ylamino)pyrimidin-4-ylamino)benzenesulfonamide;
4-(tert-butylsulfonyl)-3-(6-(5-chloropyridin-2-ylamino)pyrimidin-4-ylamino)-N-methylbenzenesulfonamide;
N-methyl-4-(propane-2-sulfonyl)-3-[6-(5-trifluoromethyl-pyridin-2-ylamino)-pyrimidin-4-ylamino]-benzenesulfonamide;
3-[6-(5-chloro-pyridin-2-ylamino)-pyrimidin-4-ylamino]-N-methyl-4-(propane-2-sulfonyl)-benzenesulfonamide;
3-({6-[(5-chloro-2-pyridinyl)amino]-4-pyrimidinyl}amino)-N-methyl-4-[(trifluoromethyl)oxy]benzenesulfonamide;
1-[6-(5-chloro-pyridin-2-ylamino)-pyrimidin-4-yl]-3,3-dimethyl-2,3-dihydro-1H-indole-6-sulfonic acid methylamide;
5-(6-(5-chloropyridin-2-ylamino)pyrimidin-4-ylamino)-2-fluoro-N-methyl-4-(1,1,1-trifluoropropan-2-yloxy)benzenesulfonamide;
5-[6-(5-chloro-pyridin-2-ylamino)-pyrimidin-4-ylamino]-2-fluoro-4-methanesulfonyl-N-methyl-benzenesulfonamide;
5-({6-[(5-chloro-2-pyridinyl)amino]-4-pyrimidinyl}amino)-2-fluoro-N-methyl-4-[(2,2,2-trifluoroethyl)oxy]benzenesulfonamide;
2-fluoro-N-methyl-4-[(2,2,2-trifluoroethyl)oxy]-5-[(6-{[5-(trifluoromethyl)-2-pyridinyl]amino}-4-pyrimidinyl)amino]benzenesulfonamide;
3-({6-[(5-fluoro-2-pyridinyl)amino]-4-pyrimidinyl}amino)-N-methyl-4-[(2,2,2-trifluoroethyl)oxy]benzenesulfonamide;
3-({6-[(5-chloro-2-pyridinyl)amino]-4-pyrimidinyl}amino)-4-(ethylsulfonyl)-N-methylbenzenesulfonamide;
4-(ethylsulfonyl)-N-methyl-3-[(6-{[5-(trifluoromethyl)-2-pyridinyl]amino}-4-pyrimidinyl)amino]benzenesulfonamide;
3-({6-[(5-cyano-2-pyridinyl)amino]-4-pyrimidinyl}amino)-N-methyl-4-(methylsulfonyl)benzenesulfonamide;
3-({6-[(5-cyano-2-pyridinyl)amino]-4-pyrimidinyl}amino)-N-methyl-4-[(2,2,2-trifluoro-1-methylethyl)oxy]benzenesulfonamide;
2-{[6-({3-[(methylamino)sulfonyl]phenyl}amino)-4-pyrimidinyl]amino}-1,3-thiazole-5-carboxylic acid;
(2-{[6-({3-[(methylamino)sulfonyl]phenyl}amino)-4-pyrimidinyl]amino}-1,3-thiazol-4-yl)acetic acid;
1-{6-[(4-chlorophenyl)amino]-4-pyrimidinyl}-N-methyl-1H-indole-6-sulfonamide;
3-{6-[(4-chlorophenyl)amino]-4-pyrimidinyl}-N-methyl-2-oxo-2,3-dihydro-1H-benzimidazole-5-sulfonamide;
3-{[6-({3-[6-(dimethylamino)-3-pyridinyl]phenyl}amino)-4-pyrimidinyl]amino}-N-methylbenzenesulfonamide;
N-methyl-3-({6-[(5-methyl-3-biphenylyl)amino]-4-pyrimidinyl}amino)benzenesulfonamide;
N-methyl-3-[(6-{[3-methyl-5-(3-pyridinyl)phenyl]amino}-4-pyrimidinyl)amino]benzenesulfonamide;
3-[(6-{[3′-(dimethylamino)-3-biphenylyl]amino}-4-pyrimidinyl)amino]-N-methylbenzenesulfonamide;
N-methyl-3-[(6-{[4′-(4-morpholinyl)-3-biphenylyl]amino}-4-pyrimidinyl)amino]-benzenesulfonamide;
N-methyl-3-{[6-({3-[6-(methyloxy)-3-pyridinyl]phenyl}amino)-4-pyrimidinyl]amino}-benzenesulfonamide;
3′-{[6-({3-[(methylamino)sulfonyl]phenyl}amino)-4-pyrimidinyl]amino}-4-biphenylcarboxamide;
N-methyl-3-{[6-({3-[5-(methyloxy)-3-pyridinyl]phenyl}amino)-4-pyrimidinyl]amino}-benzenesulfonamide;
3′-{[6-({3-[(methylamino)sulfonyl]phenyl}amino)-4-pyrimidinyl]amino}-3-biphenylcarboxamide;
N-methyl-3-{[6-({3′-[(methylsulfonyl)amino]-3-biphenylyl}amino)-4-pyrimidinyl]amino}benzenesulfonamide;
3-[(6-{[4′-(dimethylamino)-3-biphenylyl]amino}-4-pyrimidinyl)amino]-N-methylbenzenesulfonamide;
N-methyl-3-{[6-({3-[4-(methyloxy)-3-pyridinyl]phenyl}amino)-4-pyrimidinyl]amino}-benzenesulfonamide;
N-(3-{[6-({3-[(methylamino)sulfonyl]phenyl}amino)-4-pyrimidinyl]amino}-4-biphenylyl)acetamide;
N-methyl-3-{[6-({4′-[(methylsulfonyl)amino]-3-biphenylyl}amino)-4-pyrimidinyl]amino}-benzenesulfonamide;
N-(3-{[6-({3-[(methylamino)sulfonyl]phenyl}amino)-4-pyrimidinyl]amino}-3-biphenylyl)acetamide;
N-methyl-3′-{[6-({3-[(methylamino)sulfonyl]phenyl}amino)-4-pyrimidinyl]amino}-4-biphenylsulfonamide;
N-methyl-3′-{[6-({3-[(methylamino)sulfonyl]phenyl}amino)-4-pyrimidinyl]amino}-3-biphenylsulfonamide;
3-[(6-{[4-chloro-3-(3-pyridinyl)phenyl]amino}-4-pyrimidinyl)amino]-N-methylbenzenesulfonamide;
2′-chloro-5′-{[6-({3-[(methylamino)sulfonyl]phenyl}amino)-4-pyrimidinyl]amino}-3-biphenylcarboxamide;
3-[(6-{[6-chloro-3′-(4-morpholinyl)-3-biphenylyl]amino}-4-pyrimidinyl)amino]-N-methylbenzenesulfonamide;
4-{[6-({3-[(methylamino)sulfonyl]phenyl}amino)-4-pyrimidinyl]amino}benzoic acid;
[(3-{[6-({3-[(methylamino)sulfonyl]phenyl}amino)-4-pyrimidinyl]amino}phenyl)oxy]acetic acid;
N,N-dimethyl-4-{[6-({3-[(methylamino)sulfonyl]phenyl}amino)-4-pyrimidinyl]amino}benzamide;
N,N-dimethyl-2-[(3-{[6-({3-[(methylamino)sulfonyl]phenyl}amino)-4-pyrimidinyl]amino}phenyl)oxy]acetamide;
N-(2-hydroxyethyl)-4-{[6-({3-[(methylamino)sulfonyl]phenyl}amino)-4-pyrimidinyl]amino}benzamide;
N-methyl-3-{[6-({4-[(4-methyl-1-piperazinyl)carbonyl]phenyl}amino)-4-pyrimidinyl]amino}benzenesulfonamide;
4-{[6-({3-[(methylamino)sulfonyl]phenyl}amino)-4-pyrimidinyl]amino}-N-(1-methyl-4-piperidinyl)benzamide;
N-methyl-3-[(6-{[4-(1-piperazinylcarbonyl)phenyl]amino}-4-pyrimidinyl)amino]benzenesulfonamide;
N-methyl-3-[(6-{[4-({4-[2-(methyloxy)ethyl]-1-piperazinyl}carbonyl)phenyl]amino}-4-pyrimidinyl)amino]benzenesulfonamide;
4-{[6-({3-[(methylamino)sulfonyl]phenyl}amino)-4-pyrimidinyl]amino}-N-[2-(methyloxy)ethyl]benzamide;
4-{[6-({3-[(methylamino)sulfonyl]phenyl}amino)-4-pyrimidinyl]amino}-N-[3-(methyloxy)propyl]benzamide;
N-[2-(dimethylamino)ethyl]-4-{[6-({3-[(methylamino)sulfonyl]phenyl}amino)-4-pyrimidinyl]amino}benzamide;
N,N-diethyl-4-{[6-({3-[(methylamino)sulfonyl]phenyl}amino)-4-pyrimidinyl]amino}benzamide;
N-methyl-3-[(6-{[4-(1-pyrrolidinylcarbonyl)phenyl]amino}-pyrimidinyl)amino]benzenesulfonamide;
3-({6-[(4-{[(3S)-3-(dimethylamino)-1-pyrrolidinyl]carbonyl}phenyl)amino]-4-pyrimidinyl}amino)-N-methylbenzenesulfonamide;
N-methyl-3-{[6-({4-[(4-methylhexahydro-1H-1,4-diazepin-1-yl)carbonyl]phenyl}amino)-4-pyrimidinyl]amino}benzenesulfonamide;
N-methyl-3-[(6-{[4-(4-thiomorpholinylcarbonyl)phenyl]amino}-4-pyrimidinyl)amino]benzenesulfonamide;
3-{[6-({4-[(4,4-difluoro-1-piperidinyl)carbonyl]phenyl}amino)-4-pyrimidinyl]amino}-N-methylbenzenesulfonamide;
3-({6-[(4-{[(3R)-3-(dimethylamino)-1-pyrrolidinyl]carbonyl}phenyl)amino]-4-pyrimidinyl}amino)-N-methylbenzenesulfonamide;
N-[2-(dimethylamino)ethyl]-N-methyl-4-{[6-({3-[(methylamino)sulfonyl]phenyl}amino)-4-pyrimidinyl]amino}benzamide;
N-[2-(dimethylamino)ethyl]-N-methyl-4-[(6-{[5-[(methylamino)sulfonyl]-2-(methylthio)phenyl]amino}-4-pyrimidinyl)amino]benzamide;
N-[(4-{[6-({3-[(methylamino)sulfonyl]phenyl}amino)-4-pyrimidinyl]amino}phenyl)carbonyl]glycine;
N-methyl-3-[(6-{[3-(6-oxo-1,6-dihydro-3-pyridinyl)phenyl]amino}-4-pyrimidinyl)amino]benzenesulfonamide;
3-({6-[(3-hydroxyphenyl)amino]-4-pyrimidinyl}amino)-N-methylbenzenesulfonamide;
N-methyl-4-(methylsulfonyl)-3-[(6-{[4-(trifluoromethyl)phenyl]amino}-4-pyrimidinyl)amino]benzenesulfonamide;
3-({6-[(4-chlorophenyl)amino]-4-pyrimidinyl}amino)-N-methyl-4-(methylsulfonyl)benzenesulfonamide;
3-(6-(4-chlorophenylamino)pyrimidin-4-ylamino)-4-(isobutylsulfonyl)-N-methylbenzenesulfonamide;
3-(6-(4-chlorophenylamino)pyrimidin-4-ylamino)-4-(ethylsulfonyl)-N-methylbenzenesulfonamide;
3-({6-[(4-chlorophenyl)amino]-4-pyrimidinyl}amino)-N-methyl-4-[(2,2,2-trifluoro-1-methylethyl)oxy]benzenesulfonamide;
3-({6-[(4-chlorophenyl)amino]-4-pyrimidinyl}amino)-N-methyl-4-[(2,2,2-trifluoro-1-methylethyl)oxy]benzenesulfonamide;
3-({6-[(5-chloro-2-pyridinyl)amino]-4-pyrimidinyl}amino)-N-methyl-4-[(2,2,2-trifluoro-1-methylethyl)oxy]benzenesulfonamide; or
3-({6-[(5-chloro-2-pyridinyl)amino]-4-pyrimidinyl}amino)-N-methyl-4-[(2,2,2-trifluoro-1-methylethyl)oxy]benzenesulfonamide;
or a salt thereof.

12. A pharmaceutical composition comprising the compound or salt according to claim 1 and a pharmaceutically-acceptable excipient.

13. A method for treating congestive heart failure comprising administering to a patient in need thereof an effective amount of the compound or salt according to claim 1.

14. A method for treating congestive heart failure comprising administering to a patient in need thereof the pharmaceutical composition according to claim 12.
